(19) United States
(12) Patent Application Publication
(10) Pub. No.: US 2021/0257556 A1
(43) Pub. Date: Aug. 19, 2021

(54) ORGANIC ELECTROLUMINESCENT COMPOUND, A PLURALITY OF HOST MATERIALS, AND ORGANIC ELECTROLUMINESCENT DEVICE COMPRISING THE SAME

(71) Applicant: ROHM AND HAAS ELECTRONIC MATERIALS KOREA LTD., Chungcheongnam-do (KR)

(72) Inventors: Su-Hyun Lee, Gyeonggi-do (KR); So-Young Jung, Gyeonggi-do (KR); Jin-Ri Hong, Gyeonggi-do (KR); Sang-Hee Cho, Gyeonggi-do (KR); Young-Jae Kim, Gyeonggi-do (KR)

(21) Appl. No.: 17/162,993

(22) Filed: Jan. 29, 2021

(30) Foreign Application Priority Data
Jan. 31, 2020 (KR) 10-2020-0011973
Nov. 27, 2020 (KR) 10-2020-0162154

(51) Int. Cl. H01L 51/00

(52) U.S. Cl. CPC H01L 51/0069; H01L 51/006; H01L 51/0061; H01L 51/0054; H01L 51/5056; H01L 51/0072; H01L 51/0073; H01L 51/0074; H01L 51/0094; H01L 51/0067

(57) ABSTRACT

The present disclosure relates to a plurality of host materials comprising a first host material comprising a compound represented by formula 1, and a second host material comprising a compound represented by formula 2, and an organic electroluminescent device comprising the same. By comprising the specific combination of compounds as host materials, it is possible to provide an organic electroluminescent device having higher luminous efficiency and/or improved lifespan properties as compared with conventional organic electroluminescent devices.

### TECHNICAL FIELD

The present disclosure relates to an organic electroluminescent compound, a plurality of host materials, and an organic electroluminescent device comprising the same.

### BACKGROUND ART

In 1987, Tang et al. of Eastman Kodak first developed a small molecule green organic electroluminescent device (OLED) of TPD/Alq3 bilayer consisting of a light-emitting layer and a charge transport layer. Since then, the research on an OLED has been rapidly carried out, and it has been commercialized. At present, phosphorescent materials, which provide excellent luminous efficiency in realizing panels, are mainly used in OLEDs. Thus, an OLED which has high luminous efficiency and/or long lifetime is required for long time uses and high resolution of displays.

Korean Patent Appln. Laid-Open No. 2017-0022865 discloses a new organic electroluminescent compound. However, the aforementioned reference does not specifically disclose the specific combination of host materials claimed in the present disclosure. In addition, there is a need for the development of a light-emitting material having improved performances, for example, higher luminous efficiency and/or improved lifetime properties, by combining the compound disclosed in the aforementioned reference with a specific compound.

### DISCLOSURE OF INVENTION

### Technical Problem

The objective of the present disclosure is to provide an organic electroluminescent compound having a new structure suitable to apply to an organic electroluminescent device. Another objective of the present disclosure is to provide improved host materials capable of producing an organic electroluminescent device having high luminous efficiency and/or long lifetime properties.

### Solution to Problem

The present inventors found that the above objective can be achieved by a compound represented by the following formula 2. The compound represented by the following formula 2 can be applied to an organic electroluminescent device as a plurality of host materials in combination with the compound represented by the following formula 1.

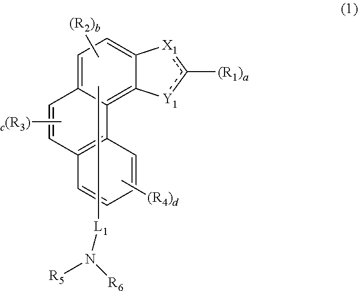

In formula 1,

X₁ and Y₁, each independently, represent —N═, —NR₇—, —O—, or —S—, with the proviso that any one of X₁ and Y₁ represents —N═, and the other of X₁ and Y₁ represents —NR₇—, —O—, or —S—;

R₁ represents a substituted or unsubstituted (C6-C30)aryl, or a substituted or unsubstituted (3- to 30-membered)heteroaryl;

R₂ to R₇, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring group of a (C3-C30) aliphatic ring(s) and a (C6-C30) aromatic ring(s), a substituted or unsubstituted mono- or di-(C1-C30)alkylamino, a substituted or unsubstituted mono- or di-(C2-C30)alkenylamino, a substituted or unsubstituted (C1-C30)alkyl(C2-C30)alkenylamino, a substituted or unsubstituted mono- or di-(C6-C30)arylamino, a substituted or unsubstituted (C1-C30)alkyl(C6-C30)arylamino, a substituted or unsubstituted mono- or di-(3- to 30-membered)heteroarylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, or a substituted or unsubstituted (C6-C30)aryl(3- to 30-membered)heteroarylamino; or may be linked to an adjacent substituent to form a ring(s);

L represents a single bond, a substituted or unsubstituted (C6-C30)arylene, or a substituted or unsubstituted (3- to 30-membered)heteroarylene; and a represents 1; b and c, each independently, represent 1 or 2; d represents an integer of 1 to 4; where if each of b to d is an integer of 2 or more, each of R₂ to R₄ may be the same or different.

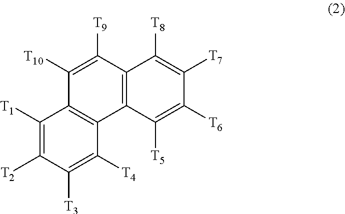

In formula 2,

T₅ and T₆ are linked to each other to form a ring represented by the following formula 3; or T₇ and T₈ are linked to each other to form a ring represented by the following formula 3; or T₅ and T₆ are linked to each other to form a ring represented by the following formula 3, and T₇ and T₈ are also linked to each other to form a ring represented b the following formula 3:

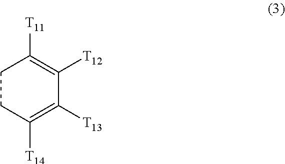

wherein,

T₁ to T₄, T₉ to T₁₄, and T₅ to T₈ not forming a ring, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arysilyl, a substituted or unsubstituted tri(C6-C30)arylsilyl, a substituted or unsubstituted fused ring group of a (C3-C30) aliphatic ring(s) and a (C6-C30) aromatic ring(s), a substituted or unsubstituted mono- or di-(C1-C30)alkylamino, a substituted or unsubstituted mono- or di-(C2-C30)alkenylamino, a substituted or unsubstituted (C1-C30)alkyl(C2-C30)alkenylamino, a substituted or unsubstituted mono- or di-(C6-C30)arylamino, a substituted or unsubstituted (C1-C30)alkyl(C6-C30)arylamino, a substituted or unsubstituted mono- or di-(3- to 30-membered)heteroarylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, a substituted or unsubstituted (C6-C30)aryl(3- to 30-membered)heteroarylamino, or -L₂-Ar₂; with the proviso that at least one of T₁ to T₁₄ represents -L₂-Ar₂;

L₂, each independently, represents a single bond, a substituted or unsubstituted (C6-C30)arylene, or a substituted or unsubstituted (3- to 30-membered)heteroarylene;

Ar₂, each independently, represents a substituted or unsubstituted (C6-C30)aryl, or a substituted or unsubstituted (3- to 30-membered)heteroaryl;

[symbol] represents a fusion site with formula 2; and

the heteroaryl contains at least one heteroatom selected from the group consisting of B, N, O, S, Si, and P.

### Advantageous Effects of Invention

The organic electroluminescent compound according to the present disclosure exhibits the performances suitable to be used in an organic electroluminescent device. In addition, by comprising the specific combination of compounds of the present disclosure as host materials, it is possible to provide an organic electroluminescent device having improved luminous efficiency and/or lifetime properties compared to conventional organic electroluminescent devices, and manufacture a display system or a lighting system using the same.

### MODE FOR THE INVENTION

Hereinafter, the present disclosure will be described in detail. However, the following description is intended to explain the disclosure, and is not meant in any way to restrict the scope of the disclosure.

The term “organic electroluminescent material” in the present disclosure means a material that may be used in an organic electroluminescent device, and may comprise at least one compound. The organic electroluminescent material may be comprised in any layer constituting an organic electroluminescent device, as necessary. For example, the organic electroluminescent material may be a hole injection material, a hole transport material, a hole auxiliary material, a light-emitting auxiliary material, an electron blocking material, a light-emitting material (containing host and dopant materials), an electron buffer material, a hole blocking material, an electron transport material, an electron injection material, etc.

The term “a plurality of organic electroluminescent materials” in the present disclosure means an organic electroluminescent material(s) comprising a combination of at least two compounds, which may be comprised in any layer constituting an organic electroluminescent device. It may mean both a material before being comprised in an organic electroluminescent device (for example, before vapor deposition) and a material after being comprised in an organic electroluminescent device (for example, after vapor deposition). For example, a plurality of organic electroluminescent materials may be a combination of at least two compounds which may be comprised in at least one of a hole injection layer, a hole transport layer, a hole auxiliary layer, a light-emitting auxiliary layer, an electron blocking layer, a light-emitting layer, an electron buffer layer, a hole blocking layer, an electron transport layer, and an electron injection layer. The at least two compounds may be comprised in the same layer or different layers, and may be mixture-evaporated or co-evaporated, or may be individually evaporated.

The term “a plurality of host materials” in the present disclosure means an organic electroluminescent material comprising a combination of at least two host materials. It may mean both a material before being comprised in an organic electroluminescent device (for example, before vapor deposition) and a material after being comprised in an organic electroluminescent device (for example, after vapor deposition). The plurality of host materials of the present disclosure may be comprised in any light-emitting layer constituting an organic electroluminescent device. The two or more compounds comprised in the plurality of host materials of the present disclosure may be comprised in one light-emitting layer or may be respectively comprised in different light-emitting layers. When two or more host materials are comprised in one layer, for example, the layer may be formed by a mixture-evaporation, or may be formed by a separate co-evaporation simultaneously.

Herein, the term “(C1-C30)alkyl” is meant to be a linear or branched alkyl having 1 to 30 carbon atoms constituting the chain, in which the number of carbon atoms is preferably 1 to 20, and more preferably 1 to 10. The above alkyl may include methyl, ethyl, n-propyl, isopropyl, n-butyl, isobutyl, sec-butyl, tert-butyl, etc. The term “(C2-C30)alkenyl” is meant to be a linear or branched alkenyl having 2 to 30 carbon atoms constituting the chain, in which the number of carbon atoms is preferably 2 to 20, and more preferably 2 to 10. The above alkenyl may include vinyl, 1-propenyl, 2-propenyl, 1-butenyl, 2-butenyl, 3-butenyl, 2-methylbut-2-enyl, etc. The term “(C2-C30)alkynyl” is meant to be a linear or branched alkynyl having 2 to 30 carbon atoms constituting the chain, in which the number of carbon atoms is preferably 2 to 20, and more preferably 2 to 10. The above alkynyl may include ethynyl, 1-propynyl, 2-propynyl, 1-butynyl, 2-butynyl, 3-butynyl, 1-methylpent-2-ynyl, etc. The term “(C3-C30)cycloalkyl” is meant to be a mono- or polycyclic hydrocarbon having 3 to 30 ring backbone carbon atoms, in which the number of carbon atoms is preferably 3 to 20, and more preferably 3 to 7. The above cycloalkyl may include cyclopropyl, cyclobutyl, cyclopentyl, cyclohexyl, cyclopentylmethyl, cyclohexylmethyl, etc. The term “(3- to 7-membered)heterocycloalkyl” is meant to be a cycloalkyl having 3 to 7, preferably 5 to 7, ring backbone atoms, and including at least one heteroatom selected from the group consisting of B, N, O, S, Si, and P, and preferably the group consisting of O, S, and N. The above heterocycloalkyl may include tetrahydrofuran, pyrrolidine, thiolan, tetrahydropyran, etc. The term “(C6-C30)aryl(ene)” is meant to be a monocyclic or fused ring radical derived from an aromatic hydrocarbon having 6 to 30 ring backbone carbon atoms. The above aryl(ene) may be partially saturated, and may comprise a spiro structure. The number of ring backbone carbon atoms is preferably 6 to 20, and more preferably 6 to 15. The above aryl may include phenyl, biphenyl, terphenyl, naphthyl, binaphthyl, phenylnaphthyl, naphthylphenyl, fluorenyl, phenylfluorenyl, diphenylfluorenyl, benzofluorenyl, dibenzofluorenyl, phenanthrenyl, phenylphenanthrenyl, anthracenyl, indenyl, triphenylenyl, pyrenyl, tetracenyl, perylenyl, chrysenyl, naphthacenyl, fluoranthenyl, spirobifluorenyl, spiro[fluorene-benzofluoren]yl, azulenyl, etc. More specifically, the aryl may include phenyl, 1-naphthyl, 2-naphthyl, 1-anthryl, 2-anthryl, 9-anthryl, benzanthryl, 1-phenanthryl, 2-phenanthryl, 3-phenanthryl, 4-phenanthryl, 9-phenanthryl, naphthacenyl, pyrenyl, 1-chrysenyl, 2-chrysenyl, 3-chrysenyl, 4-chrysenyl, 5-chrysenyl, 6-chrysenyl, benzo[c]phenanthryl, benzo[g]chrysenyl, 1-triphenylenyl, 2-triphenylenyl, 3-triphenylenyl, 4-triphenylenyl, 1-fluorenyl, 2-fluorenyl, 3-fluorenyl, 4-fluorenyl, 9-fluorenyl, benzo[a]fluorenyl, benzo[b]fluorenyl, benzo[c]fluorenyl, dibenzofluorenyl, 2-biphenylyl, 3-biphenylyl, 4-biphenylyl, o-terphenyl, m-terphenyl-4-yl, m-terphenyl-3-yl, m-terphenyl-2-yl, p-terphenyl-4-yl, p-terphenyl-3-yl, p-terphenyl-2-yl, m-quaterphenyl, 3-fluoranthenyl, 4-fluoranthenyl, 8-fluoranthenyl, 9-fluoranthenyl, benzofluoranthenyl, o-tolyl, m-tolyl, p-tolyl, 2,3-xylyl, 3,4-xylyl, 2,5-xylyl, mesityl, o-cumenyl, m-cumenyl, p-cumenyl, p-tert-butylphenyl, p-(2-phenylpropyl)phenyl, 4′-methylbiphenylyl, 4″-tert-butyl-p-terphenyl-4-yl, 9,9-dimethyl-1-fluorenyl, 9,9-dimethyl-2-fluorenyl, 9,9-dimethyl-3-fluorenyl, 9,9-dimethyl-4-fluorenyl, 9,9-diphenyl-1-fluorenyl, 9,9-diphenyl-2-fluorenyl, 9,9-diphenyl-3-fluorenyl, 9,9-diphenyl-4-fluorenyl, 11,11-dimethyl-1-benzo[a]fluorenyl, 11,11-dimethyl-2-benzo[a]fluorenyl, 11,11-dimethyl-3-benzo[a]fluorenyl, 11,11-dimethyl-4-benzo[a]fluorenyl, 11,11-dimethyl-5-benzo[a]fluorenyl, 11,11-dimethyl-6-benzo[a]fluorenyl, 11,11-dimethyl-7-benzo[a]fluorenyl, 11,11-dimethyl-8-benzo[a]fluorenyl, 11,11-dimethyl-9-benzo[a]fluorenyl, 11,11-dimethyl-10-benzo[a]fluorenyl, 11,11-dimethyl-1-benzo[b]fluorenyl, 11,11-dimethyl-2-benzo[b]fluorenyl, 11,11-dimethyl-3-benzo[b]fluorenyl, 11,11-dimethyl-4-benzo[b]fluorenyl, 11,11-dimethyl-5-benzo[b]fluorenyl, 11,11-dimethyl-6-benzo[b]fluorenyl, 11,11-dimethyl-7-benzo[b]fluorenyl, 11,11-dimethyl-8-benzo[b]fluorenyl, 11,11-dimethyl-9-benzo[b]fluorenyl, 11,11-dimethyl-10-benzo[b]fluorenyl, 11,11-dimethyl-1-benzo[c]fluorenyl, 11,11-dimethyl-2-benzo[c]fluorenyl, 11,11-dimethyl-3-benzo[c]fluorenyl, 11,11-dimethyl-4-benzo[c]fluorenyl, 11,11-dimethyl-5-benzo[c]fluorenyl, 11,11-dimethyl-6-benzo[c]fluorenyl, 11,11-dimethyl-7-benzo[c]fluorenyl, 11,11-dimethyl-8-benzo[c]fluorenyl, 11,11-dimethyl-9-benzo[c]fluorenyl, 11,11-dimethyl-10-benzo[c]fluorenyl, 11,11-diphenyl-1-benzo[a]fluorenyl, 11,11-diphenyl-2-benzo[a]fluorenyl, 11,11-diphenyl-3-benzo[a]fluorenyl, 11,11-diphenyl-4-benzo[a]fluorenyl, 11,11-diphenyl-5-benzo[a]fluorenyl, 11,11-diphenyl-6-benzo[a]fluorenyl, 11,11-diphenyl-7-benzo[a]fluorenyl, 11,11-diphenyl-8-benzo[a]fluorenyl, 11,11-diphenyl-9-benzo[a]fluorenyl, 11,11-diphenyl-10-benzo[a]fluorenyl, 11,11-diphenyl-1-benzo[b]fluorenyl, 11,11-diphenyl-2-benzo[b]fluorenyl, 11,11-diphenyl-3-benzo[b]fluorenyl, 11,11-diphenyl-4-benzo[b]fluorenyl, 11,11-diphenyl-5-benzo[b]fluorenyl, 11,11-diphenyl-6-benzo[b]fluorenyl, 11,11-diphenyl-7-benzo[b]fluorenyl, 11,11-diphenyl-8-benzo[b]fluorenyl, 11,11-diphenyl-9-benzo[b]fluorenyl, 11,11-diphenyl-10-benzo[b]fluorenyl, 11,11-diphenyl-1-benzo[c]fluorenyl, 11,11-diphenyl-2-benzo[c]fluorenyl, 11,11-diphenyl-3-benzo[c]fluorenyl, 11,11-diphenyl-4-benzo[c]fluorenyl, 11,11-diphenyl-5-benzo[c]fluorenyl, 11,11-diphenyl-6-benzo[c]fluorenyl, 11,11-diphenyl-7-benzo[c]fluorenyl, 11,11-diphenyl-8-benzo[c]fluorenyl, 11,11-diphenyl-9-benzo[c]fluorenyl, 11,11-diphenyl-10-benzo[c]fluorenyl, 9,9,10,10-tetramethyl-9,10-dihydro-1-phenanthrenyl, 9,9,10,10-tetramethyl-9,10-dihydro-2-phenanthrenyl, 9,9,10,10-tetramethyl-9,10-dihydro-3-phenanthrenyl, 9,9,10,10-tetramethyl-9,10-dihydro-4-phenanthrenyl, etc.

The term “(3- to 30-membered)heteroaryl(ene)” is an aryl(ene) having 3 to 30 ring backbone atoms, preferably 3 to 25 ring backbone atoms, more preferably 5 to 20 ring backbone atoms, and including at least one, preferably 1 to 4 heteroatoms selected from the group consisting of B, N, O, S, Si, and P. The above heteroaryl(ene) may be a monocyclic ring, or a fused ring condensed with at least one benzene ring; may be partially saturated; may be one formed by linking at least one heteroaryl or aryl group to a heteroaryl group via a single bond(s); and may comprise a spiro structure. The above heteroaryl may include a monocyclic ring-type heteroaryl such as furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, pyridazinyl, etc., and a fused ring-type heteroaryl such as benzofuranyl, benzothiophenyl, isobenzofuranyl, dibenzofuranyl, benzonaphthofuranyl, dibenzothiophenyl, dibenzoselenophenyl, benzonaphthothiophenyl, benzofuroquinolinyl, benzofuroquinazolinyl, benzofuronaphthyridinyl, benzofuropyrimidinyl, naphthofuropyrimidinyl, benzothienoquinolinyl, benzothienoquinazolinyl, benzothienonaphthyridinyl, benzothienopyrimidinyl, naphthothienopyrimidinyl, pyrimidoindolyl, benzopyrimidoindolyl, benzofuropyrazinyl, naphthofuropyrazinyl, benzothienopyrazinyl, naphthothienopyrazinyl, pyrazinoindolyl, benzopyrazinoindolyl, benzimidazolyl, benzothiazolyl, benzoisothiazolyl, benzoisoxazolyl, benzoxazolyl, isoindolyl, indolyl, indazolyl, benzothiadiazolyl, quinolyl, isoquinolyl, cinnolinyl, quinazolinyl, quinoxalinyl, naphthyrdinyl, carbazolyl, benzocarbazolyl, phenoxazinyl, phenanthridinyl, phenanthro oxazolyl, benzodioxolyl, dihydroacridinyl, benzotrazolphenazinyl, imidazopyridyl, chromenoquinazolinyl, thiochromenoquinazolinyl, dimethylbenzopermidinyl, indolocarbazolyl, indenocarbazolyl, etc. More specifically, the heteroaryl may include 1-pyrrolyl, 2-pyrrolyl, 3-pyrrolyl, pyrazinyl, 2-pyrdyl, 2-pyrimidinyl, 4-pyrmidinyl, 5-pyrmidinyl, 6-pyrmidinyl, 1,2,3-trazin-4-yl, 1,2,4-trazin-3-yl, 1,3,5-triazin-2-yl, 1-imidazolyl, 2-imidazolyl, 1-pyrazolyl, 1-indolidinyl, 2-indolidinyl, 3-indolidinyl, 5-indolidinyl, 6-indolidinyl, 7-indolidinyl, 8-indolidinyl, 2-imidazopyridyl, 3-imidazopyridyl, 5-imidazopyridyl, 6-imidazopyridyl, 7-imidazopyridyl, 8-imidazopyridyl, 3-pyrdyl, 4-pyridyl, 1-indolyl, 2-indolyl, 3-indolyl, 4-indolyl, 5-indolyl, 6-indolyl, 7-indolyl, 1-isoindolyl, 2-isoindolyl, 3-isoindolyl, 4-isoindolyl, 5-isoindolyl, 6-isoindolyl, 7-isoindolyl, 2-furyl, 3-furyl, 2-benzofuranyl, 3-benzofuranyl, 4-benzofuranyl, 5-benzofuranyl, 6-benzofuranyl, 7-benzofuranyl, 1-isobenzofuranyl, 3-isobenzofuranyl, 4-isobenzofuranyl, 5-isobenzofuranyl, 6-isobenzofuranyl, 7-isobenzofuranyl, 2-quinolyl, 3-quinolyl, 4-quinolyl, 5-quinolyl, 6-quinolyl, 7-quinolyl, 8-quinolyl, 1-isoquinolyl, 3-isoquinolyl, 4-isoquinolyl, 5-isoquinolyl, 6-isoquinolyl, 7-isoquinolyl, 8-isoquinolyl, 2-quinoxalinyl, 5-quinoxalinyl, 6-quinoxalinyl, 1-carbazolyl, 2-carbazolyl, 3-carbazolyl, 4-carbazolyl, 9-carbazolyl, azacarbazolyl-1-yl, azacarbazolyl-2-yl, azacarbazolyl-3-yl, azacarbazolyl-4-yl, azacarbazolyl-5-yl, azacarbazolyl-6-yl, azacarbazolyl-7-yl, azacarbazolyl-8-yl, azacarbazolyl-9-yl, 1-phenanthridinyl, 2-phenanthridinyl, 3-phenanthridinyl, 4-phenanthridinyl, 6-phenanthridinyl, 7-phenanthridinyl, 8-phenanthridinyl, 9-phenanthridinyl, 10-phenanthridinyl, 1-acridinyl, 2-acridinyl, 3-acridinyl, 4-acridinyl, 9-acridinyl, 2-oxazolyl, 4-oxazolyl, 5-oxazolyl, 2-oxadiazolyl, 5-oxadiazolyl, 3-furazanyl, 2-thienyl, 3-thienyl, 2-methylpyrrol-1-yl, 2-methylpyrrol-3-yl, 2-methylpyrrol-4-yl, 2-methylpyrrol-5-yl, 3-methylpyrrol-1-yl, 3-methylpyrrol-2-yl, 3-methylpyrrol-4-yl, 3-methylpyrrol-5-yl, 2-tert-butylpyrrol-4-yl, 3-(2-phenylpropyl)pyrrol-1-yl, 2-methyl-1-indolyl, 4-methyl-1-indolyl, 2-methyl-3-indolyl, 4-methyl-3-indolyl, 2-tert-butyl-1-indolyl, 4-tert-butyl-1-indolyl, 2-tert-butyl-3-indolyl, 4-tert-butyl-3-indolyl, 1-dibenzofuranyl, 2-dibenzofuranyl, 3-dibenzofuranyl, 4-dibenzofuranyl, 1-dibenzothiophenyl, 2-dibenzothiophenyl, 3-dibenzothiophenyl, 4-dibenzothiophenyl, 1-naphtho-[1,2-b]-benzofuranyl, 2-naphtho-[1,2-b]-benzofuranyl, 3-naphtho-[1,2-b]-benzofuranyl, 4-naphtho-[1,2-b]-benzofuranyl, 5-naphtho-[1,2-b]-benzofuranyl, 6-naphtho-[1,2-b]-benzofuranyl, 7-naphtho-[1,2-b]-benzofuranyl, 8-naphtho-[1,2-b]-benzofuranyl, 9-naphtho-[1,2-b]-benzofuranyl, 10-naphtho-[1,2-b]-benzofuranyl, 1-naphtho-[2,3-b]-benzofuranyl, 2-naphtho-[2,3-b]-benzofuranyl, 3-naphtho-[2,3-b]-benzofuranyl, 4-naphtho-[2,3-b]-benzofuranyl, 5-naphtho-[2,3-b]-benzofuranyl, 6-naphtho-[2,3-b]-benzofuranyl, 7-naphtho-[2,3-b]-benzofuranyl, 8-naphtho-[2,3-b]-benzofuranyl, 9-naphtho-[2,3-b]-benzofuranyl, 10-naphtho-[2,3-b]-benzofuranyl, 1-naphtho-[2,1-b]-benzofuranyl, 2-naphtho-[2,1-b]-benzofuranyl, 3-naphtho-[2,1-b]-benzofuranyl, 4-naphtho-[2,1-b]-benzofuranyl, 5-naphtho-[2,1-b]-benzofuranyl, 6-naphtho-[2,1-b]-benzofuranyl, 7-naphtho-[2,1-b]-benzofuranyl, 8-naphtho-[2,1-b]-benzofuranyl, 9-naphtho-[2,1-b]-benzofuranyl, 10-naphtho-[2,1-b]-benzofuranyl, 1-naphtho-[1,2-b]-benzothiophenyl, 2-naphtho-[1,2-b]-benzothiophenyl, 3-naphtho-[1,2-b]-benzothiophenyl, 4-naphtho-[1,2-b]-benzothiophenyl, 5-naphtho-[1,2-b]-benzothiophenyl, 6-naphtho-[1,2-b]-benzothiophenyl, 7-naphtho-[1,2-b]-benzothiophenyl, 8-naphtho-[1,2-b]-benzothiophenyl, 9-naphtho-[1,2-b]-benzothiophenyl, 10-naphtho-[1,2-b]-benzothiophenyl, 1-naphtho-[2,3-b]-benzothiophenyl, 2-naphtho-[2,3-b]-benzothiophenyl, 3-naphtho-[2,3-b]-benzothiophenyl, 4-naphtho-[2,3-b]-benzothiophenyl, 5-naphtho-[2,3-b]-benzothiophenyl, 1-naphtho-[2,1-b]-benzothiophenyl, 2-naphtho-[2,1-b]-benzothiophenyl, 3-naphtho-[2,1-b]-benzothiophenyl, 4-naphtho-[2,1-b]-benzothiophenyl, 5-naphtho-[2,1-b]-benzothiophenyl, 6-naphtho-[2,1-b]-benzothiophenyl, 7-naphtho-[2,1-b]-benzothiophenyl, 8-naphtho-[2,1-b]-benzothiophenyl, 9-naphtho-[2,1-b]-benzothiophenyl, 10-naphtho-[2,1-b]-benzothiophenyl, 2-benzofuro[3,2-d]pyrimidinyl, 6-benzofuro[3,2-d]pyrimidinyl, 7-benzofuro[3,2-d]pyrmidinyl, 8-benzofuro[3,2-d]pyrimidinyl, 9-benzofuro[3,2-d]pyrimidinyl, 2-benzothio[3,2-d]pyrmidinyl, 6-benzothio[3,2-d]pyrmidinyl, 7-benzothio[3,2-d]pyrimidinyl, 8-benzothio[3,2-d]pyrimidinyl, 9-benzothio[3,2-d]pyrmidinyl, 2-benzofuro[3,2-d]pyrazinyl, 6-benzofuro[3,2-d]pyrazinyl, 7-benzofuro[3,2-d]pyrazinyl, 8-benzofuro[3,2-d]pyrazinyl, 9-benzofuro[3,2-d]pyrazinyl, 2-benzothio[3,2-d]pyrazinyl, 6-benzothio[3,2-d]pyrazinyl, 7-benzothio[3,2-d]pyrazinyl, 8-benzothio[3,2-d]pyrazinyl, 9-benzothio[3,2-d]pyrazinyl, 1-silafluorenyl, 2-silafluorenyl, 3-silafluorenyl, 4-silafluorenyl, 1-germafluorenyl, 2-germafluorenyl, 3-germafluorenyl, 4-germafluorenyl, 1-dibenzoselenophenyl, 2-dibenzoselenophenyl, 3-dibenzoselenophenyl, 4-dibenzoselenophenyl, etc. “Halogen” includes F, C1, Br, and I.

In addition, “ortho (o-),” “meta (m-),” and “para (p-)” are prefixes, which represent the relative positions of substituents, respectively. Ortho indicates that two substituents are adjacent to each other, and for example, when two substituents in a benzene derivative occupy positions 1 and 2, it is called an ortho position. Meta indicates that two substituents are at positions 1 and 3, and for example, when two substituents in a benzene derivative occupy positions 1 and 3, it is called a meta position. Para indicates that two substituents are at positions 1 and 4, and for example, when two substituents in a benzene derivative occupy positions 1 and 4, it is called a para position.

In the formulas of the present disclosure, a ring formed by a linkage of adjacent substituents means that at least two adjacent substituents are linked to or fused with each other to form a substituted or unsubstituted mono- or polycyclic (3- to 30-membered) alicyclic or aromatic ring, or the combination thereof; preferably, a substituted or unsubstituted mono- or polycyclic (3- to 26-membered) alicyclic or aromatic ring, or the combination thereof; more preferably, an unsubstituted mono- or polycyclic (5- to 20-membered) aromatic ring. Also, the ring may contain at least one heteroatom selected from B, N, O, S, Si, and P, preferably at least one heteroatom selected from N, O, and S. For example, the ring may be a substituted or unsubstituted, benzene ring, indene ring, indole ring, benzoindole ring, benzofuran ring, benzothiophene ring, etc.

Herein, “substituted” in the expression “substituted or unsubstituted” means that a hydrogen atom in a certain functional group is replaced with another atom or another functional group, i.e., a substituent, and also includes that the hydrogen atom is replaced with a group formed by a linkage of two or more substituents. For example, the group formed by a linkage of two or more substituents may be pyridine-triazine. That is, pyridine-triazine may be interpreted as a heteroaryl substituent, or as substituents in which two heteroaryl substituents are linked. In the present disclosure, the substituent(s) of the substituted alkyl, the substituted aryl, the substituted arylene, the substituted heteroaryl, the substituted heteroarylene, the substituted cycloalkyl, the substituted alkoxy, the substituted trialkylsilyl, the substituted dialkylarylsilyl, the substituted alkyldiarylsilyl, the substituted triarylsilyl, the substituted fused ring group of a aliphatic ring(s) and a aromatic ring(s), the substituted mono- or di-alkylamino, the substituted mono- or di-alkenylamino, the substituted alkylalkenylamino, the substituted mono- or di-arylamino, the substituted alkylarylamino, the substituted mono- or di-heteroarylamino, the substituted alkylheteroarylamino, the substituted alkenylarylamino, the substituted alkenylheteroarylamino, and the substituted arylheteroarylamino, each independently, are at least one selected from the group consisting of deuterium; a halogen: a cyano; a carboxyl; a nitro; a hydroxyl; a phosphine oxide; a (C1-C30)alkyl; a halo(C1-C30)alkyl; a (C2-C30)alkenyl; a (C2-C30)alkynyl; a (C1-C30)alkoxy; a (C1-C30)alkylthio; a (C3-C30)cycloalkyl; a (C3-C30)cycloalkenyl; a (3- to 7-membered)heterocycloalkyl; a (C6-C30)aryloxy; a (C6-C30)arylthio; a (3- to 30-membered)heteroaryl unsubstituted or substituted with at least one of a (C1-C30)alkyl(s) and a (C6-C30)aryl(s); a (C6-C30)aryl unsubstituted or substituted with at least one of deuterium, a cyano(s), a (C1-C30)alkyl(s), a (C3-C30)cycloalkyl(s), a tri(C1-C30)alkylsilyl(s), and a (3- to 30-membered)heteroaryl(s); a tri(C1-C30)alkylsilyl; a tri(C6-C30)arylsilyl; a di(C1-C30)alkyl(C6-C30)arylsilyl; a (C1-C30)alkyldi(C6-C30)arylsilyl; a fused ring group of a (C3-C30) aliphatic ring(s) and a (C6-C30) aromatic ring(s); an amino; a mono- or di-(C1-C30)alkylamino; a mono- or di-(C2-C30)alkenylamino; a (C1-C30)alkyl(C2-C30)alkenylamino; a mono- or di-(C6-C30)arylamino; a (C1-C30)alkyl(C6-C30)arylamino; a mono- or di-(3- to 30-membered)heteroarylamino; a (C1-C30)alkyl(3- to 30-membered)heteroarylamino; a (C2-C30)alkenyl(C6-C30)arylamino; a (C2-C30)alkenyl(3- to 30-membered)heteroarylamino; a (C6-C30)aryl(3- to 30-membered)heteroarylamino; a (C1-C30)alkylcarbonyl; a (C1-C30)alkoxycarbonyl; a (C6-C30)arylcarbonyl; a (C6-C30)arylphosphine; a di(C6-C30)arylboronyl; a di(C1-C30)alkylboronyl; a (C1-C30)alkyl(C6-C30)arylboronyl; a (C6-C30)aryl(C1-C30)alkyl; and a (C1-C30)alkyl(C6-C30)aryl. According to one embodiment of the present disclosure, the substituent(s), each independently, are at least one selected from the group consisting of a (C1-C20)alkyl; a (C6-C25)cycloalkyl; a (5- to 25-membered)heteroaryl unsubstituted or substituted with a (C6-C25)aryl(s); a (C6-C25)aryl unsubstituted or substituted with at least one of deuterium, a cyano(s), a (C1-C20)alkyl(s), a (C6-25)cycloalkyl(s), a tri(C1-C20)alkylsilyl(s), and a (5- to 25-membered)heteroaryl(s); a mono- or di-(C6-C25)arylamino; a mono- or di-(5- to 25-membered)heteroarylamino: and a (C6-C25)aryl(5- to 25-membered)heteroarylamino. According to another embodiment of the present disclosure, the substituent(s), each independently, are at least one selected from the group consisting of a (C1-C10)alkyl; a (C6-C18)cycloalkyl; a (5- to 20-membered)heteroaryl unsubstituted or substituted with a (C6-C18)aryl(s); a (C6-C25)aryl unsubstituted or substituted with at least one of deuterium, a cyano(s), a (C1-C10)alkyl(s), a (C6-18)cycloalkyl(s), a tri(C1-C10)alkylsilyl(s), and a (5- to 20-membered)heteroaryl(s); a di(C6-C18)arylamino; and a (C6-C18)aryl(5- to 20-membered)heteroarylamino. For example, the substituent(s), each independently, may be at least one selected from the group consisting of a methyl; a cyclohexyl; a substituted or unsubstituted phenyl; a naphthyl; a biphenyl; a phenanthrenyl; an antracenyl; a fluoranthenyl; a naphthylphenyl; a methylfluorenyl; a dimethylfluorenyl; a diphenylfluorenyl; a terphenyl; a chrysenyl; a triphenylenyl; a spirobifluorenyl; a pyridyl substituted with a phenyl(s); a benzimidazolyl with a phenyl(s); a triazinyl substituted with a phenyl(s) and/or a naphthyl(s); a benzothiophenyl; a dibenzothiophenyl; a dibenzofuranyl unsubstituted or substituted with a phenyl(s) and/or a biphenyl(s); a carbazolyl unsubstituted or substituted with a phenyl(s); a naphtho oxazolyl substituted with a phenyl(s); a diphenylamino; a dibiphenylamino; a phenylbiphenylamino; a phenyldibenzothiofuranylamino; and a phenyldibenzothiophenylamino, and the substituent(s) of the substituted phenyl may be at least one selected from the group consisting of deuterium, a cyano, a methyl, a tert-butyl, a trimethylsilyl, a carbazolyl, and a cyclohexyl.

Herein, the heteroaryl, the heteroarylene, and the heterocycloalkyl, each independently, may contain at least one heteroatom selected from B, N, O, S, Si, and P. Also, the heteroatom may be bonded to at least one selected from the group consisting of hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arysilyl, a substituted or unsubstituted mono- or di-(C1-C30)alkylamino, a substituted or unsubstituted mono- or di-(C6-C30)arylamino, and a substituted or unsubstituted (C1-C30)alkyl(C6-C30)arylamino.

In formula 1, X₁ and Y₁, each independently, represent —N═, —NR₇—, —O—, or —S—, with the proviso that any one of X₁ and Y₁ represents —N═, and the other of X₁ and Y represents —NR₇—, —O—, or —S—. According to one embodiment, any one of X₁ and Y₁ represents —N═, and the other represents —O—, or —S—. For example, X₁ represents —N═, and Y₁ represents —O—; X₁ represents —O—, and Y₁ represents —N═; or X₁ represents —S—, and Y₁ represents —N═.

In formula 1, R₁ represents a substituted or unsubstituted (C6-C30)aryl, or a substituted or unsubstituted (3- to 30-membered)heteroaryl. According to one embodiment, R₁ represents a substituted or unsubstituted (C6-C30)aryl, or a substituted or unsubstituted (5- to 25-membered)heteroaryl. According to another embodiment, R₁ represents a substituted or unsubstituted (C6-C30)aryl, or a substituted or unsubstituted (5- to 20-membered)heteroaryl. For example, R₁ represents an unsubstituted phenyl, an unsubstituted biphenyl, an unsubstituted naphthyl, a fluorenyl substituted with a methyl(s), a benzofluorenyl substituted with a methyl(s), an unsubstituted dibenzofuranyl, an unsubstituted dibenzothiophenyl, a spiro[fluorene-fluoren]yl, a spiro[fluorene-benzofluoren]yl, or an unsubstituted pyridyl.

In formula 1, R₂ to R₇, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arysilyl, a substituted or unsubstituted tri(C6-C30)arylsilyl, a substituted or unsubstituted fused ring group of a (C3-C30) aliphatic ring(s) and a (C6-C30) aromatic ring(s), a substituted or unsubstituted mono- or di-(C1-C30)alkylamino, a substituted or unsubstituted mono- or di-(C2-C30)alkenylamino, a substituted or unsubstituted (C1-C30)alkyl(C2-C30)alkenylamino, a substituted or unsubstituted mono- or di-(C6-C30)arylamino, a substituted or unsubstituted (C1-C30)alkyl(C6-C30)arylamino, a substituted or unsubstituted mono- or di-(3- to 30-membered)heteroarylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, or a substituted or unsubstituted (C6-C30)aryl(3- to 30-membered)heteroarylamino: or may be linked to an adjacent substituent to form a ring(s). According to one embodiment, R₂ to R₇, each independently, represent hydrogen, a substituted or unsubstituted (C6-C25)aryl, a substituted or unsubstituted (3- to 25-membered)heteroaryl, or a substituted or unsubstituted mono- or di-(C6-C25)arylamino: or may be linked to an adjacent substituent to form a substituted or unsubstituted mono- or polycyclic (C3-C30) alicyclic or aromatic ring(s), whose carbon atom(s) may be replaced with at least one heteroatom selected from nitrogen, oxygen, and sulfur. According to another embodiment, R₂ to R₇, each independently, represent hydrogen, a substituted or unsubstituted (C6-C25)aryl, a substituted or unsubstituted (5- to 25-membered)heteroaryl, a substituted or unsubstituted di(C6-C18)arylamino, a substituted or unsubstituted mono- or di-(3- to 30-membered)heteroarylamino, or a substituted or unsubstituted (C6-C30)aryl(3- to 30-membered)heteroarylamino; or may be linked to an adjacent substituent to form a substituted or unsubstituted mono- or polycyclic (C3-C25) alicyclic or aromatic ring(s), whose carbon atom(s) may be replaced with at least one heteroatom selected from nitrogen and sulfur, and the heteroaryl may contain at least one heteroatom selected from the group consisting of B, N, O, S, Si, and P. Specifically, R₁, R₅, and R₆, each independently, represent a substituted or unsubstituted phenyl, a substituted or unsubstituted naphthyl, a substituted or unsubstituted biphenyl, a substituted or unsubstituted terphenyl, a substituted or unsubstituted phenanthrenyl, a substituted or unsubstituted fluorenyl, a substituted or unsubstituted benzofluorenyl, a substituted or unsubstituted triphenylenyl, a substituted or unsubstituted spirobifluorenyl, a substituted or unsubstituted pyridyl, a substituted or unsubstituted triazinyl, a substituted or unsubstituted pyrimidinyl, a substituted or unsubstituted quinolyl, a substituted or unsubstituted quinazolinyl, a substituted or unsubstituted quinoxalinyl, a substituted or unsubstituted benzoquinazolinyl, a substituted or unsubstituted benzoquinoxalinyl, a substituted or unsubstituted benzofuropyrimidinyl, a substituted or unsubstituted carbazolyl, a substituted or unsubstituted dibenzothiophenyl, a substituted or unsubstituted benzothiophenyl, a substituted or unsubstituted dibenzofuranyl, a substituted or unsubstituted benzofuranyl, a substituted or unsubstituted naphthyridinyl, a substituted or unsubstituted benzonaphthofuranyl, or a substituted or unsubstituted benzonaphthothiophenyl. For example, at least one of R₅ and Re, each independently, represent a substituted or unsubstituted phenyl, a substituted or unsubstituted o-biphenyl, a substituted or unsubstituted m-biphenyl, a substituted or unsubstituted p-biphenyl, a substituted or unsubstituted fluorenyl, a substituted or unsubstituted naphthyl, a substituted or unsubstituted phenanthrenyl, a substituted or unsubstituted dibenzofuranyl, a substituted or unsubstituted dibenzothiophenyl, or a substituted or unsubstituted benzofluorenyl. For example, R₁ may be a phenyl, a biphenyl, or a pyridyl; R₂ and R₃ may be hydrogen; Ra may be hydrogen or a phenyl; R₅ and R₆, each independently, may be a substituted phenyl, a naphthyl, a biphenyl, a phenanthrenyl, a dimethylfluorenyl, a diphenylfluorenyl, a naphthylphenyl, a phenylnaphthyl, a dimethylbenzofluorenyl, a terphenyl, a spirobifluorenyl, a benzofuranyl, a benzothiophenyl, a dibenzothiophenyl, a dibenzofuranyl unsubstituted or substituted with a phenyl(s), a carbazolyl substituted with a phenyl(s), or a benzonaphthofuranyl; and the substituent(s) of the substituted phenyl may be at least one selected from the group consisting of a phenyl substituted with at least one of deuterium, a methyl(s), and a tert-butyl(s); an antracenyl; a fluoranthenyl; a phenylfluorenyl; a cyclohexyl; a pyridyl substituted with a phenyl(s); phenoxazinyl; and a benzimidazolyl substituted with a phenyl(s).

In formula 1, a represents 1 or 2, preferably, 1; b and c, each independently, represent 1 or 2, preferably, 1; d represents an integer of 1 to 4, preferably, 1 or 2. If each of b to d is an integer of 2 or more, each of R₂ to R₄ may be the same or different.

In formula 1, L₁ represents a single bond, a substituted or unsubstituted (C6-C30)arylene, or a substituted or unsubstituted (3- to 30-membered)heteroarylene. According to one embodiment of the present disclosure, L₁ represents a single bond, or a substituted or unsubstituted (C6-C18)arylene. According to another embodiment of the present disclosure, L₁ represents a single bond, or an unsubstituted (C6-C12)arylene. For example, L₁ represents a single bond, or an unsubstituted phenylene.

According to one embodiment of the present disclosure, formula 1 may be represented by at least one of the following formulas 1-1 to 1-3.

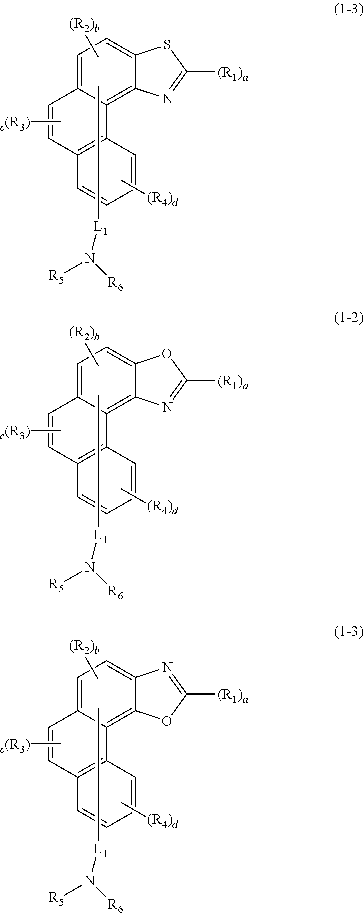

In formulas 1-1 to 1-3, R₁ to R₆, L₁, and a to d are as defined in formula 1 above.

In formula 2, T₅ and T₆ are linked to each other to form a ring represented by formula 3; or T₇ and T₈ are linked to each other to form a ring represented by formula 3; or T₅ and T₆ are linked to each other to form a ring represented by formula 3, and T₇ and T₈ are also linked to each other to forma ring represented by formula 3.

According to one embodiment of the present disclosure, T₅ and T₆ are linked to each other to form a ring represented by formula 3; or T₇ and T₈ are linked to each other to form a ring represented by formula 3. In formula 3, - - - represents a fusion site with formula 2.

In formulas 2 and 3, T₁ to T₁₄, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring group of a (C3-C30) aliphatic ring(s) and a (C6-C30) aromatic ring(s), a substituted or unsubstituted mono- or di-(C1-C30)alkylamino, a substituted or unsubstituted mono- or di-(C2-C30)alkenylamino, a substituted or unsubstituted (C1-C30)alkyl(C2-C30)alkenylamino, a substituted or unsubstituted mono- or di-(C6-C30)arylamino, a substituted or unsubstituted (C1-C30)alkyl(C6-C30)arylamino, a substituted or unsubstituted mono- or di-(3- to 30-membered)heteroarylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, a substituted or unsubstituted (C6-C30)aryl(3- to 30-membered)heteroarylamino, or -L₂-Ar₂. At least one of T₁ to T₁₄ represents -L₂-Ar₂, and preferably, any one of T₁ to T₁₄ represents -L₂-Ar₂. According to one embodiment of the present disclosure, at least one of T₁ to T₄, T₁ to T₁₄, and T₅ to T₈ not forming a ring may represent -L₂-Ar₂. According to another embodiment of the present disclosure, T₁ to T₁₄, each independently, represent hydrogen, or a substituted or unsubstituted (C6-C25)aryl, or -L₂-Ar₂. For example, T₁ to T₁₄, each independently, represent hydrogen, a phenyl, a naphthyl, a biphenyl, or -L₂-Ar₂.

L₂, each independently, represents a single bond, a substituted or unsubstituted (C6-C30)arylene, or a substituted or unsubstituted (3- to 30-membered)heteroarylene. The arylene of L₂ may represent a 1,2-phenylene, a 1,3-phenylene, a 1,4-phenylene, a 1,2-biphenylene, a 1,3-biphenylene, a 1,4-biphenylene, a 1,2-terphenylene, a 1,3-terphenylene, a 1,4-terphenylene, a 1,2-naphthylene, a 1,3-naphthylene, a 1,4-naphthylene, a 1,5-naphthylene, a 1,6-naphthylene, a 1,7-naphthylene, a 1,8-naphthylene, a 2,3-naphthylene, a 2,6-naphthylene, a 2,7-naphthylene, or a phenanthrenylene. According to one embodiment of the present disclosure, L₂, each independently, represents a single bond, or a substituted or unsubstituted (C6-C25)arylene. According to another embodiment of the present disclosure, L₂, each independently, represents a single bond, or a (C6-C18)arylene unsubstituted or substituted with a (C6-C18)aryl(s). For example, L₂, each independently, represents a single bond, a phenylene unsubstituted or substituted with a phenyl(s), a naphthylene, or a biphenylene.

Ar₂, each independently, represents a substituted or unsubstituted (C6-C30)aryl, or a substituted or unsubstituted (3- to 30-membered)heteroaryl. The heteroaryl of Ar₂ may represent a pyridyl, a pyrimidinyl, a triazinyl, a quinazolinyl, a benzoquinazolinyl, a dibenzoquinazolinyl, a quinoxalinyl, a benzoquinoxalinyl, a dibenzoquinoxalinyl, a quinolyl, a benzoquinolyl, an isoquinolyl, a naphthyridinyl, a benzoisoquinolyl, an imidazolyl, a benzimidazolyl, a phenanthroimidazolyl, a thiazolyl, a benzothiazolyl, a phenanthrothiazolyl, an oxazolyl, a benzoxazolyl, a phenanthro oxazolyl, a naphtho oxazolyl, a naphthothiazolyl, a benzothienopyrimidinyl, a benzothienopyrazinyl, a benzofuropyrimidinyl, a benzofuropyrazinyl, a benzothienoquinolyl, a benzofuroquinolyl, an acenaphthopyrimidinyl, a benzobenzofurophenanthridinyl, a carbazolyl, a dibenzofuranyl, a dibenzothiophenyl, a benzonaphthofuranyl, a benzonaphthothiophenyl, a benzocarbazolyl, a dibenzocarbazolyl, a phenoxazinyl, or a benzoperimidinyl. According to one embodiment of the present disclosure, Ar₂, each independently, represents a substituted or unsubstituted (C6-C25)aryl, or a substituted or unsubstituted (5- to 25-membered)heteroaryl. According to another embodiment of the present disclosure, Ar₂, each independently, represents a substituted or unsubstituted (5- to 25-membered)heteroaryl. For example, Ar₂, each independently, may be a substituted pyrimidinyl; a substituted triazinyl; a benzimidazolyl substituted with a phenyl(s); a quinolyl substituted with a naphthyl(s); an isoquinolyl substituted with a phenyl(s); a quinazolinyl substituted with a phenyl(s) and/or a biphenyl(s); a quinoxalinyl substituted with a phenyl(s) and/or a biphenyl(s); a naphthyridinyl substituted with a phenyl(s); a benzofuropyrimidinyl substituted with a biphenyl(s); a benzoquinoxalinyl substituted with a biphenyl(s); an acenaphthopyrimidinyl substituted with a phenyl(s); a benzoperimidinyl substituted with a methyl(s); a benzothienoquinolyl; a dibenzoquinoxalinyl; or a phenanthroimidazolyl substituted with a phenyl(s) and/or a biphenyl(s). The substituent(s) of the substituted pyrimidinyl and the substituted triazinyl may be at least one selected from the group consisting of a phenyl; a phenyl substituted with fluorine; a phenyl substituted with a tert-butyl(s); a phenyl substituted with a trimethylsilyl(s); a phenyl substituted with a carbazolyl(s); a phenyl substituted with a cyclohexyl(s); a phenyl substituted with a cyano(s); a naphthyl; a biphenyl; a terphenyl; a naphthylphenyl; a phenylnaphthyl; a phenanthrenyl; an antracenyl; a chrysenyl; a triphenylenyl; a dimethylfluorenyl; a diphenylfluorenyl; a spirobifluorenyl; a pyridyl substituted with a phenyl(s); a dibenzothiophenyl; a dibenzofuranyl; a dibenzofuranyl substituted with a phenyl(s); a dibenzofuranyl substituted with a biphenyl(s); a carbazolyl; a carbazolyl substituted with a phenyl(s); a phenoxazinyl; a benzothiophenyl; and a naphtho oxazolinyl substituted with a phenyl(s).

Formula 2 may be represented by at least one of the following formulas 2-1 and 2-2.

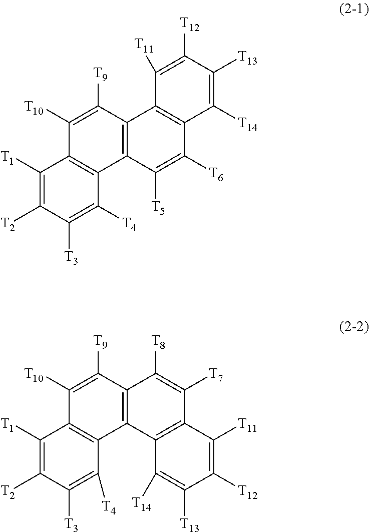

In formulas 2-1 and 2-2, T₁ to T₁₄ are as defined in formula 2 above.

Formula 2 may be represented by at least one of the following formulas.

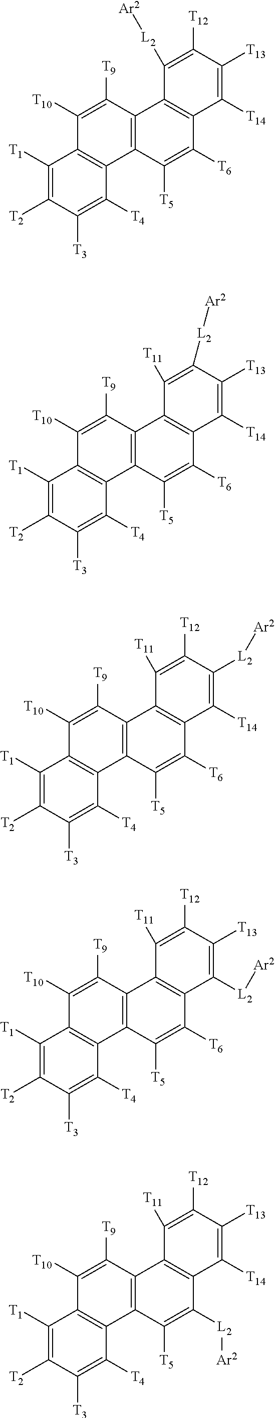

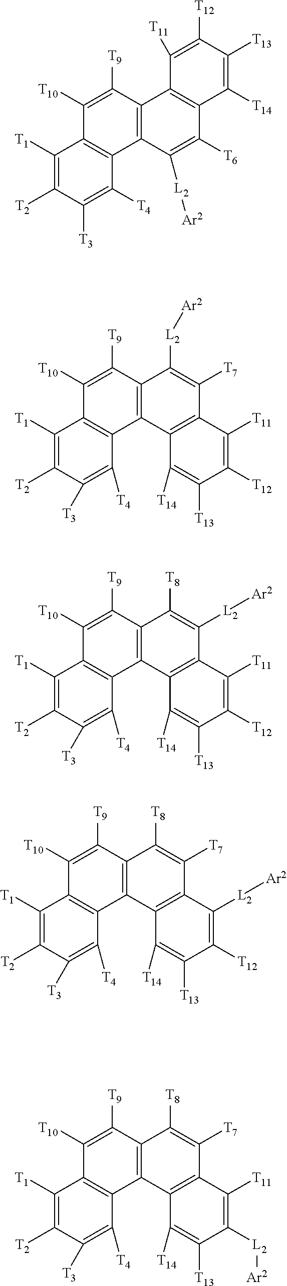

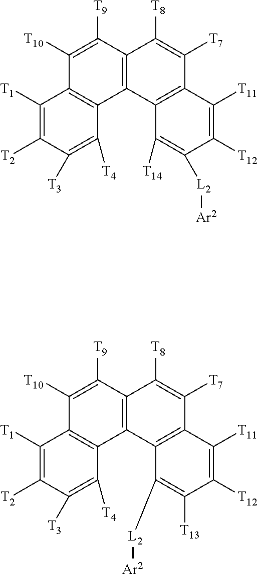

In the formulas above,

T₁ to T₁₄, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring group of a (C3-C30) aliphatic ring(s) and a (C6-C30) aromatic ring(s), a substituted or unsubstituted mono- or di-(C1-C30)alkylamino, a substituted or unsubstituted mono- or di-(C2-C30)alkenylamino, a substituted or unsubstituted (C1-C30)alkyl(C2-C30)alkenylamino, a substituted or unsubstituted mono- or di-(C6-C30)arylamino, a substituted or unsubstituted (C1-C30)alkyl(C6-C30)arylamino, a substituted or unsubstituted mono- or di-(3- to 30-membered)heteroarylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, or a substituted or unsubstituted (C6-C30)aryl(3- to 30-membered)heteroarylamino; and

L₂ and Ar₂ are as defined in formula 2 above.

The compound represented by formula 1 may be specifically exemplified by the following compounds, but is not limited thereto.

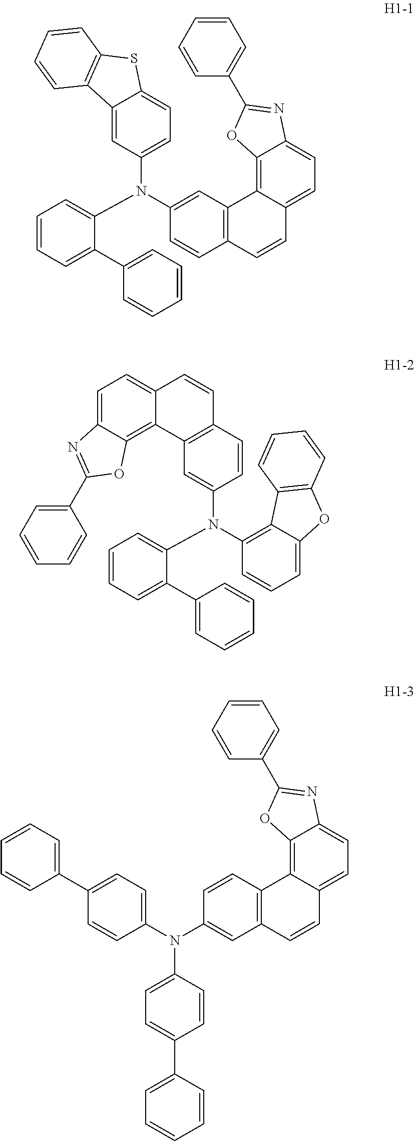 [SMILES: c1ccc(-c2nc3ccc4ccc5ccc(N(c6ccc7sc8ccccc8c7c6)c6ccccc6-c6ccccc6)cc5c4c3o2)cc1 ; c1ccc(-c2nc3ccc4ccc5ccc(N(c6ccccc6-c6ccccc6)c6cccc7oc8ccccc8c67)cc5c4c3o2)cc1 ; c1ccc(-c2ccc(N(c3ccc(-c4ccccc4)cc3)c3ccc4c(ccc5ccc6nc(-c7ccccc7)oc6c54)c3)cc2)cc1]

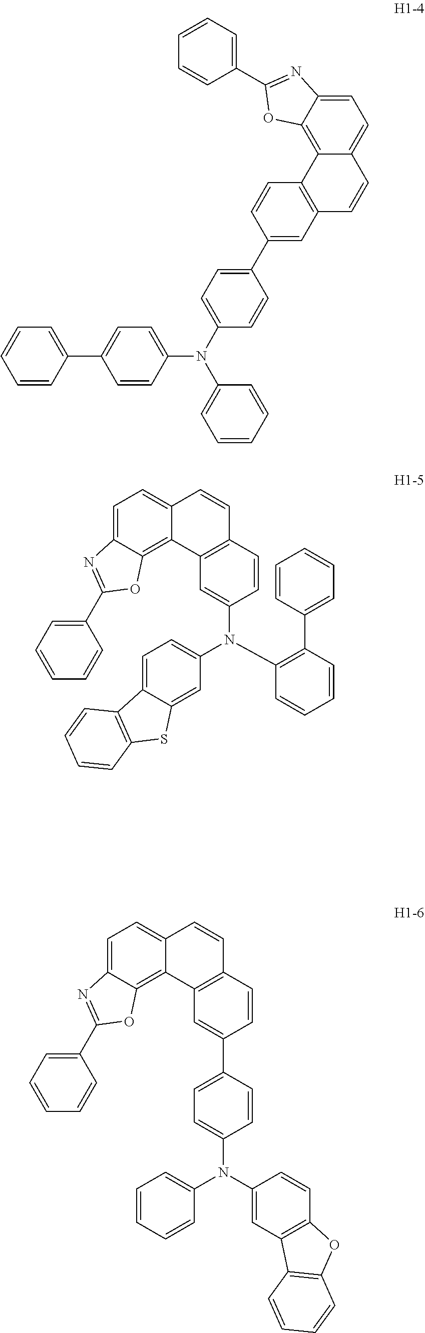 [SMILES: c1ccc(-c2ccc(N(c3ccccc3)c3ccc(-c4ccc5c(ccc6ccc7nc(-c8ccccc8)oc7c65)c4)cc3)cc2)cc1 ; c1ccc(-c2nc3ccc4ccc5ccc(N(c6ccc7c(c6)sc6ccccc67)c6ccccc6-c6ccccc6)cc5c4c3o2)cc1 ; c1ccc(-c2nc3ccc4ccc5ccc(-c6ccc(N(c7ccccc7)c7ccc8oc9ccccc9c8c7)cc6)cc5c4c3o2)cc1]

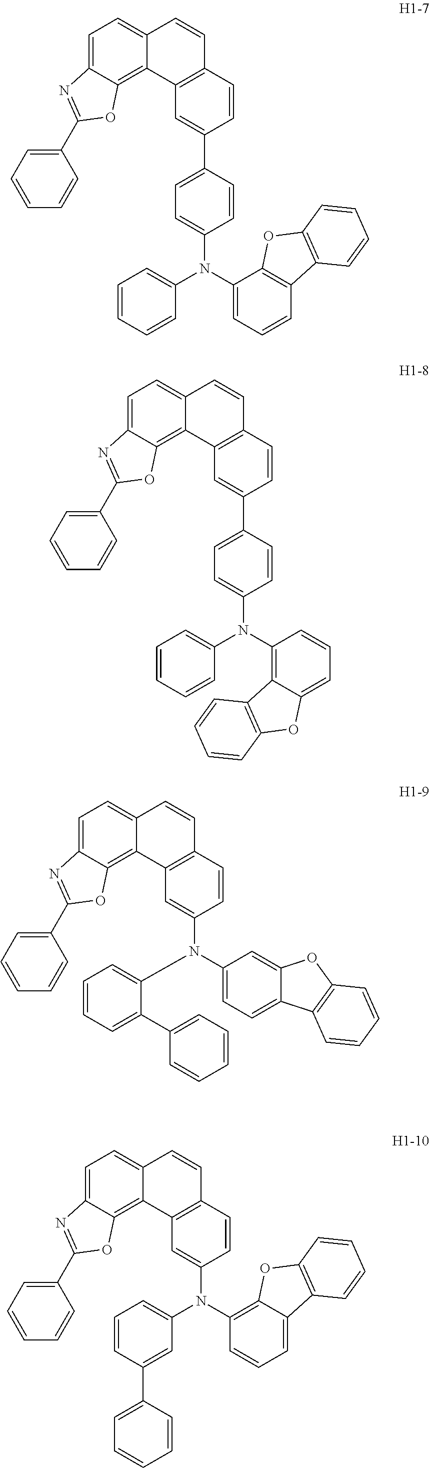

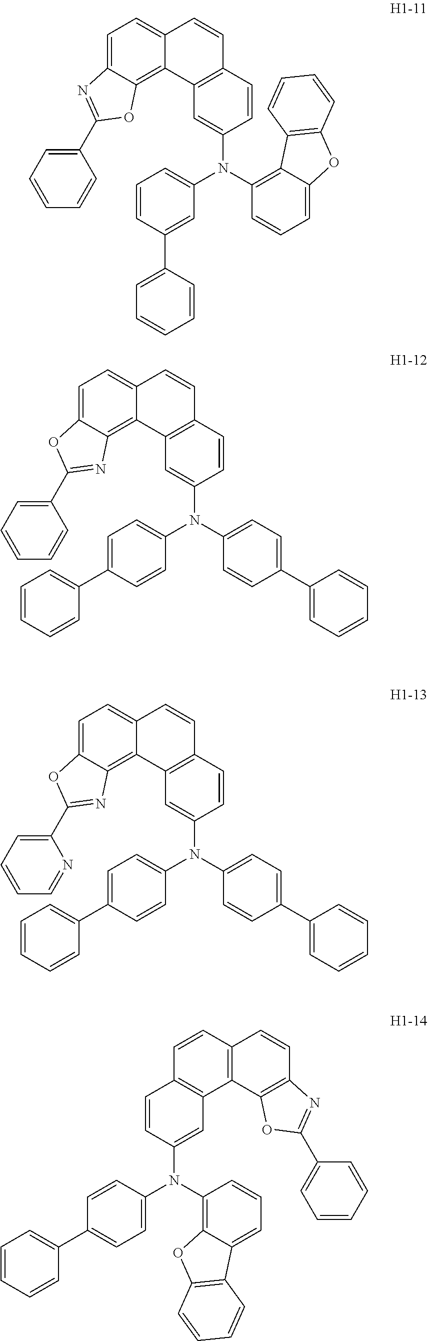

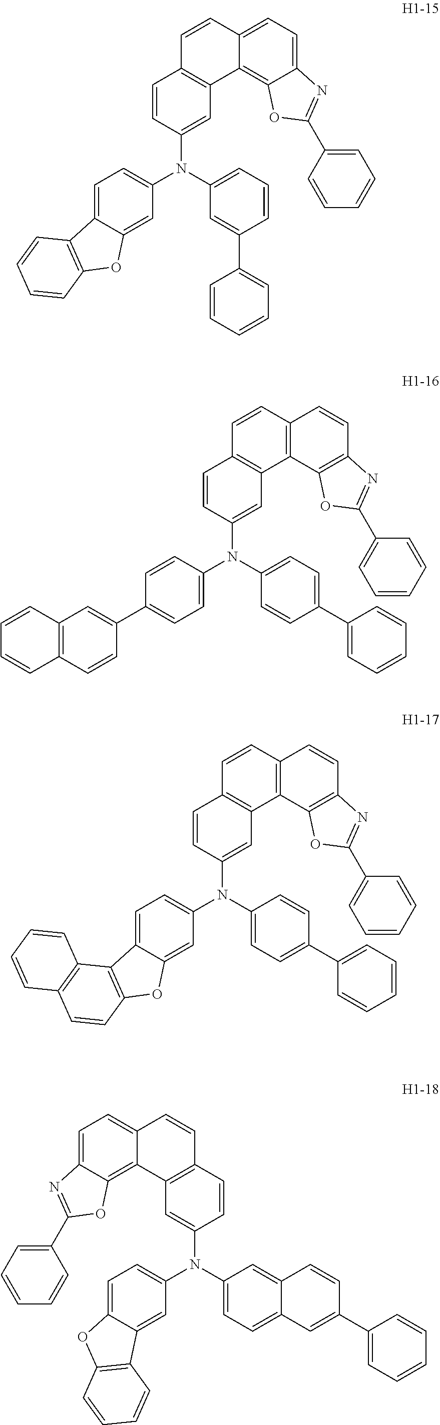 [SMILES: c1ccc(-c2cccc(N(c3ccc4c(c3)oc3ccccc34)c3ccc4ccc5ccc6nc(-c7ccccc7)oc6c5c4c3)c2)cc1 ; c1ccc(-c2ccc(N(c3ccc(-c4ccc5ccccc5c4)cc3)c3ccc4ccc5ccc6nc(-c7ccccc7)oc6c5c4c3)cc2)cc1 ; c1ccc(-c2ccc(N(c3ccc4c(c3)oc3ccc5ccccc5c34)c3ccc4ccc5ccc6nc(-c7ccccc7)oc6c5c4c3)cc2)cc1 ; c1ccc(-c2ccc3cc(N(c4ccc5oc6ccccc6c5c4)c4ccc5ccc6ccc7nc(-c8ccccc8)oc7c6c5c4)ccc3c2)cc1]

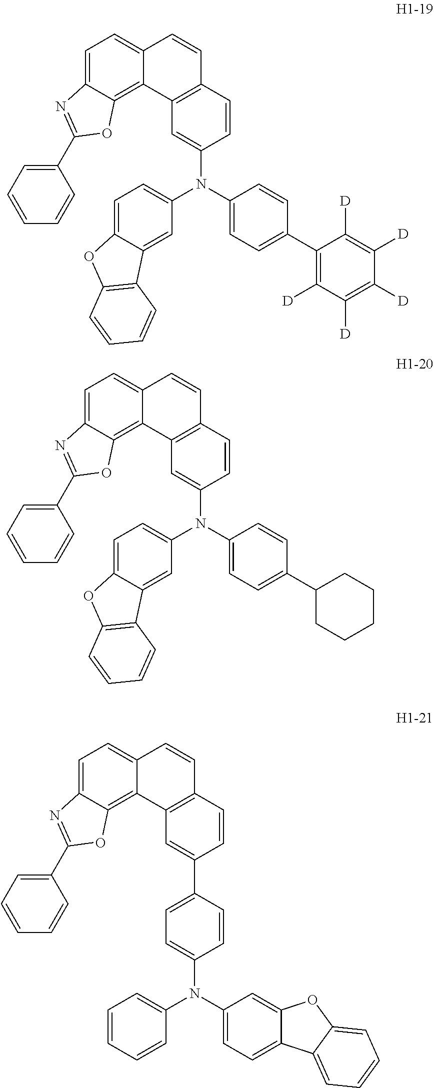

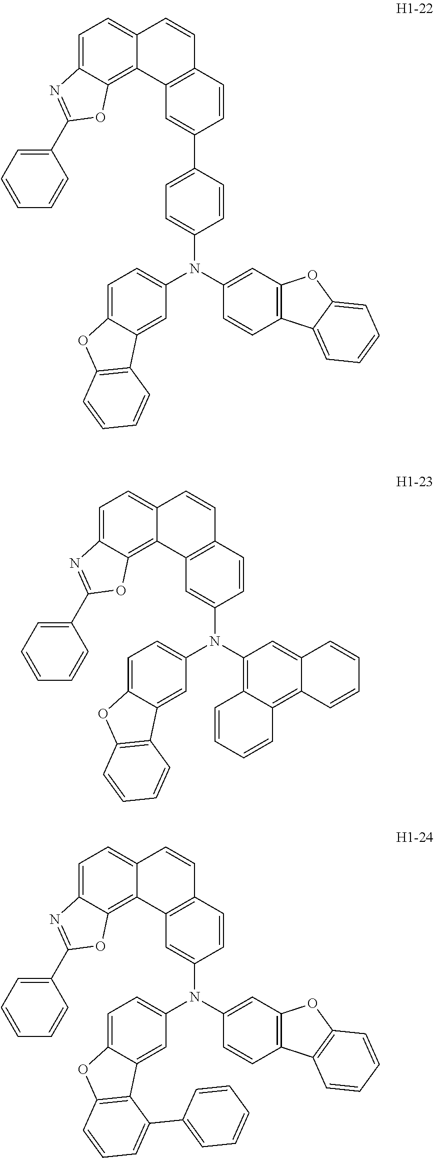 [SMILES: c1ccc(-c2nc3ccc4ccc5ccc(-c6ccc(N(c7ccc8c(c7)oc7ccccc78)c7ccc8oc9ccccc9c8c7)cc6)cc5c4c3o2)cc1 ; c1ccc(-c2nc3ccc4ccc5ccc(N(c6ccc7oc8ccccc8c7c6)c6cc7ccccc7c7ccccc67)cc5c4c3o2)cc1 ; c1ccc(-c2nc3ccc4ccc5ccc(N(c6ccc7c(c6)oc6ccccc67)c6ccc7oc8cccc(-c9ccccc9)c8c7c6)cc5c4c3o2)cc1]

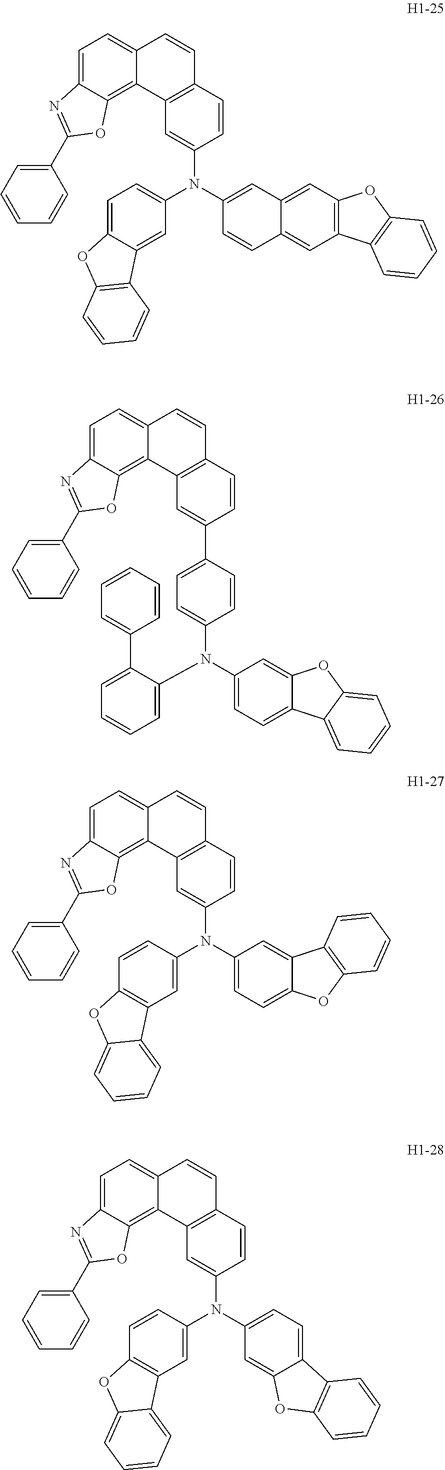 [SMILES: c1ccc(-c2nc3ccc4ccc5ccc(N(c6ccc7cc8c(cc7c6)oc6ccccc68)c6ccc7oc8ccccc8c7c6)cc5c4c3o2)cc1 ; c1ccc(-c2nc3ccc4ccc5ccc(-c6ccc(N(c7ccc8c(c7)oc7ccccc78)c7ccccc7-c7ccccc7)cc6)cc5c4c3o2)cc1 ; c1ccc(-c2nc3ccc4ccc5ccc(N(c6ccc7oc8ccccc8c7c6)c6ccc7oc8ccccc8c7c6)cc5c4c3o2)cc1 ; c1ccc(-c2nc3ccc4ccc5ccc(N(c6ccc7c(c6)oc6ccccc67)c6ccc7oc8ccccc8c7c6)cc5c4c3o2)cc1]

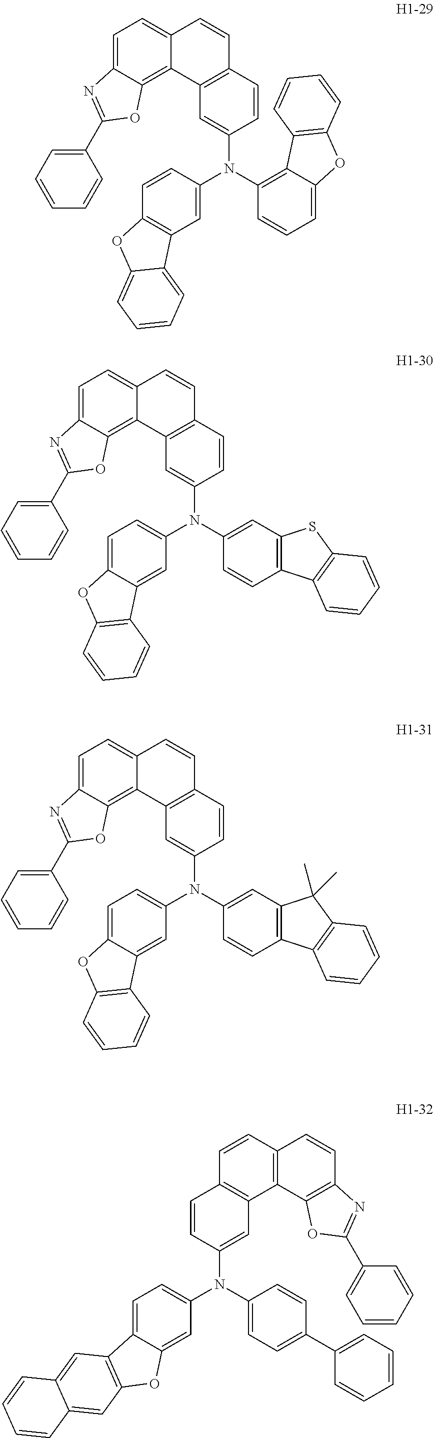

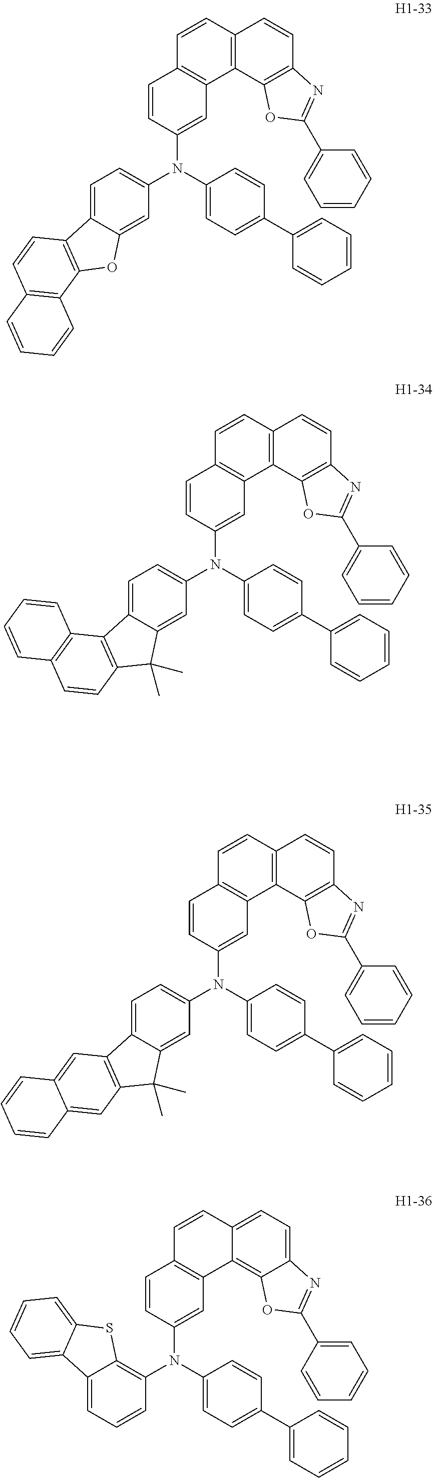 [SMILES: c1ccc(-c2ccc(N(c3ccc4c(c3)oc3c5ccccc5ccc43)c3ccc4ccc5ccc6nc(-c7ccccc7)oc6c5c4c3)cc2)cc1 ; CC1(C)c2cc(N(c3ccc(-c4ccccc4)cc3)c3ccc4ccc5ccc6nc(-c7ccccc7)oc6c5c4c3)ccc2-c2c1ccc1ccccc21 ; CC1(C)c2cc(N(c3ccc(-c4ccccc4)cc3)c3ccc4ccc5ccc6nc(-c7ccccc7)oc6c5c4c3)ccc2-c2cc3ccccc3cc21 ; c1ccc(-c2ccc(N(c3ccc4ccc5ccc6nc(-c7ccccc7)oc6c5c4c3)c3cccc4c3sc3ccccc34)cc2)cc1]

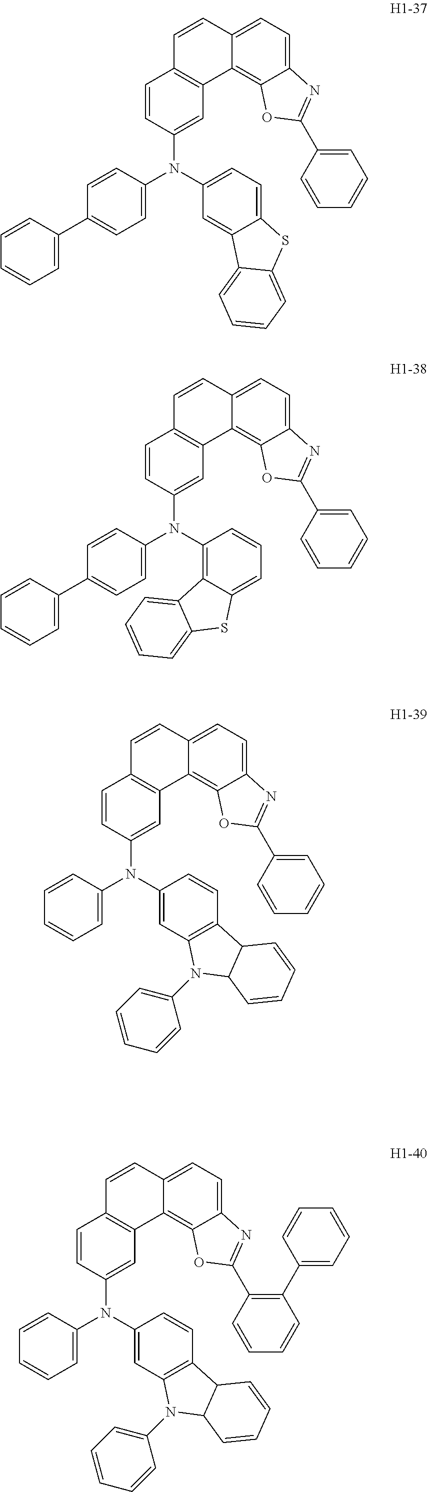 [SMILES: c1ccc(-c2ccc(N(c3ccc4sc5ccccc5c4c3)c3ccc4ccc5ccc6nc(-c7ccccc7)oc6c5c4c3)cc2)cc1 ; c1ccc(-c2ccc(N(c3ccc4ccc5ccc6nc(-c7ccccc7)oc6c5c4c3)c3cccc4sc5ccccc5c34)cc2)cc1 ; C1=CC2c3ccc(N(c4ccccc4)c4ccc5ccc6ccc7nc(-c8ccccc8)oc7c6c5c4)cc3N(c3ccccc3)C2C=C1 ; C1=CC2c3ccc(N(c4ccccc4)c4ccc5ccc6ccc7nc(-c8ccccc8-c8ccccc8)oc7c6c5c4)cc3N(c3ccccc3)C2C=C1]

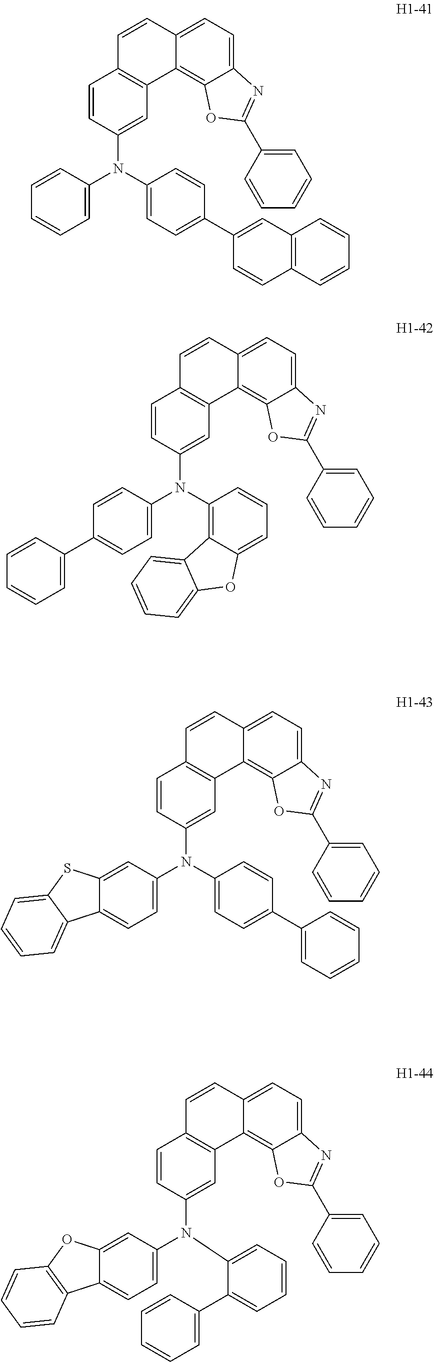 [SMILES: c1ccc(-c2nc3ccc4ccc5ccc(N(c6ccccc6)c6ccc(-c7ccc8ccccc8c7)cc6)cc5c4c3o2)cc1 ; c1ccc(-c2ccc(N(c3ccc4ccc5ccc6nc(-c7ccccc7)oc6c5c4c3)c3cccc4oc5ccccc5c34)cc2)cc1 ; c1ccc(-c2ccc(N(c3ccc4c(c3)sc3ccccc34)c3ccc4ccc5ccc6nc(-c7ccccc7)oc6c5c4c3)cc2)cc1 ; c1ccc(-c2nc3ccc4ccc5ccc(N(c6ccc7c(c6)oc6ccccc67)c6ccccc6-c6ccccc6)cc5c4c3o2)cc1]

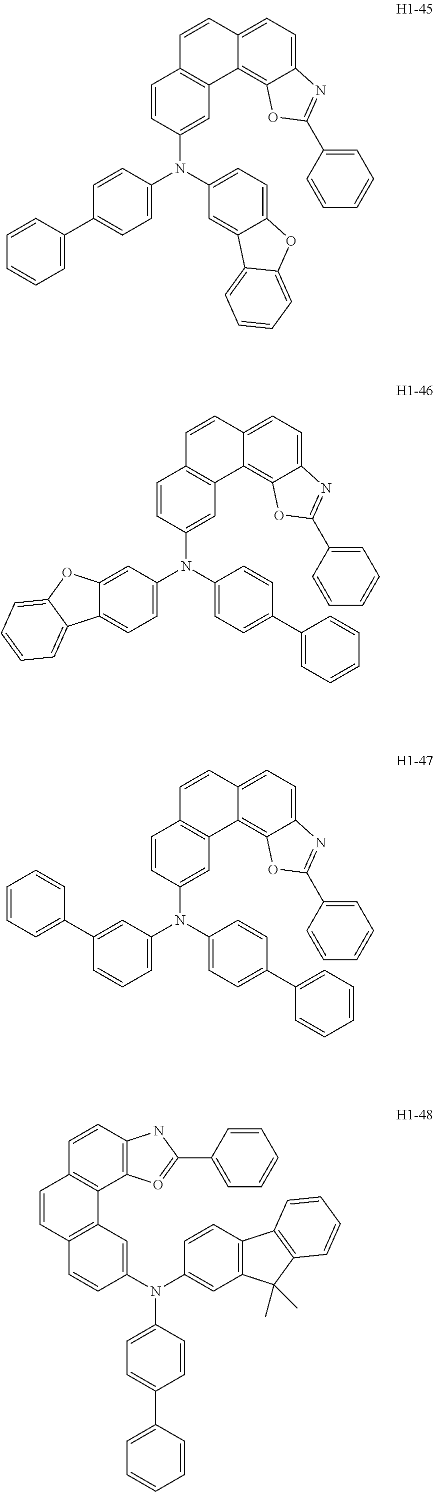

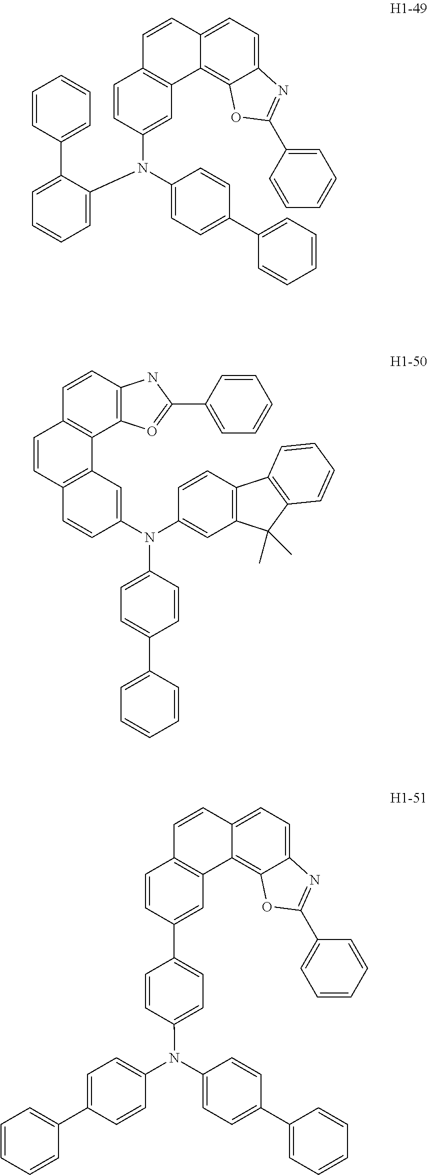

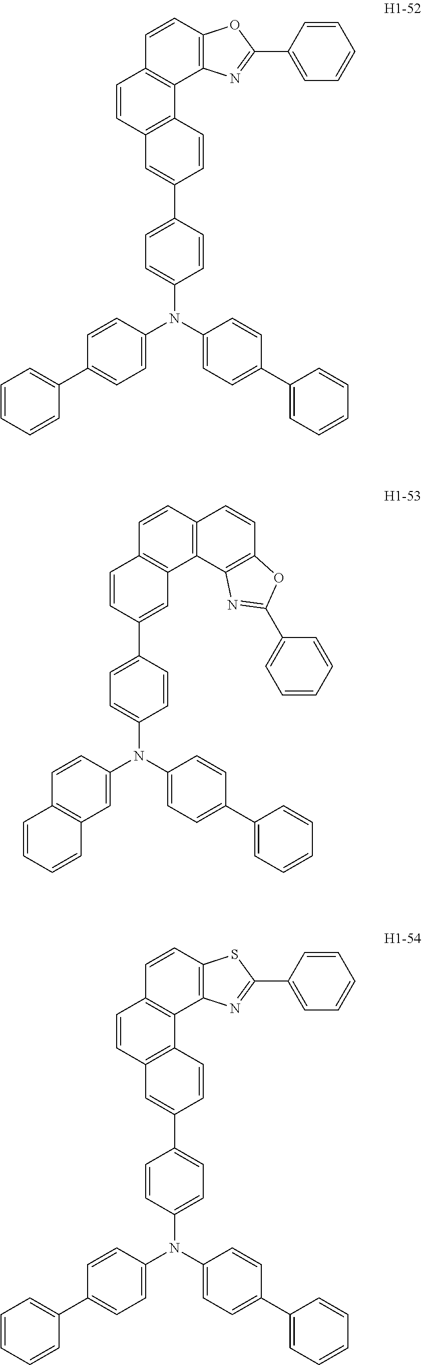 [SMILES: c1ccc(-c2ccc(N(c3ccc(-c4ccccc4)cc3)c3ccc(-c4ccc5c(ccc6ccc7oc(-c8ccccc8)nc7c65)c4)cc3)cc2)cc1 ; c1ccc(-c2ccc(N(c3ccc(-c4ccc5ccc6ccc7oc(-c8ccccc8)nc7c6c5c4)cc3)c3ccc4ccccc4c3)cc2)cc1 ; c1ccc(-c2ccc(N(c3ccc(-c4ccccc4)cc3)c3ccc(-c4ccc5c(ccc6ccc7sc(-c8ccccc8)nc7c65)c4)cc3)cc2)cc1]

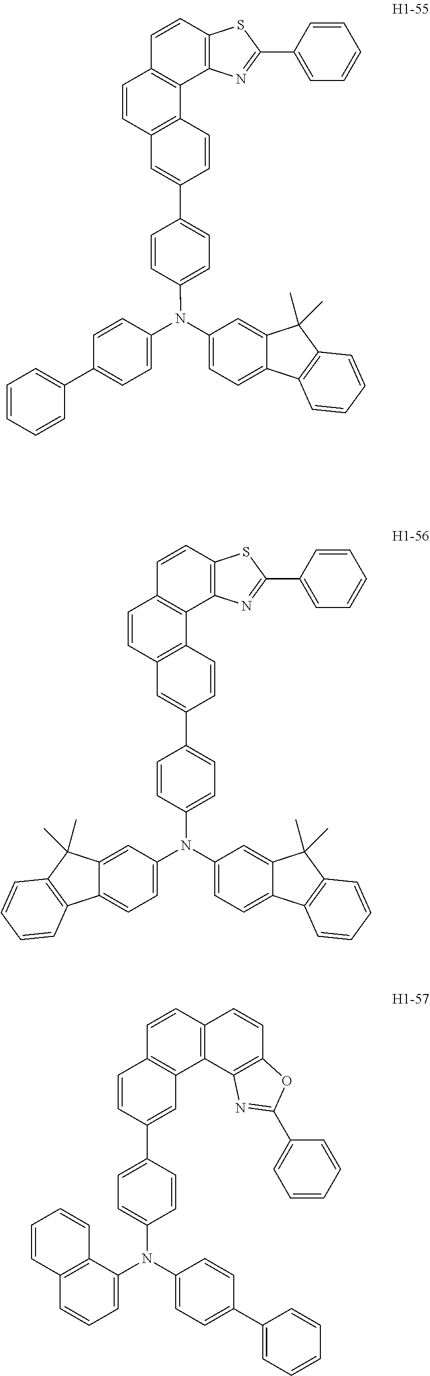 [SMILES: CC1(C)c2ccccc2-c2ccc(N(c3ccc(-c4ccccc4)cc3)c3ccc(-c4ccc5c(ccc6ccc7sc(-c8ccccc8)nc7c65)c4)cc3)cc21 ; CC1(C)c2ccccc2-c2ccc(N(c3ccc(-c4ccc5c(ccc6ccc7sc(-c8ccccc8)nc7c65)c4)cc3)c3ccc4c(c3)C(C)(C)c3ccccc3-4)cc21 ; c1ccc(-c2ccc(N(c3ccc(-c4ccc5ccc6ccc7oc(-c8ccccc8)nc7c6c5c4)cc3)c3cccc4ccccc34)cc2)cc1]

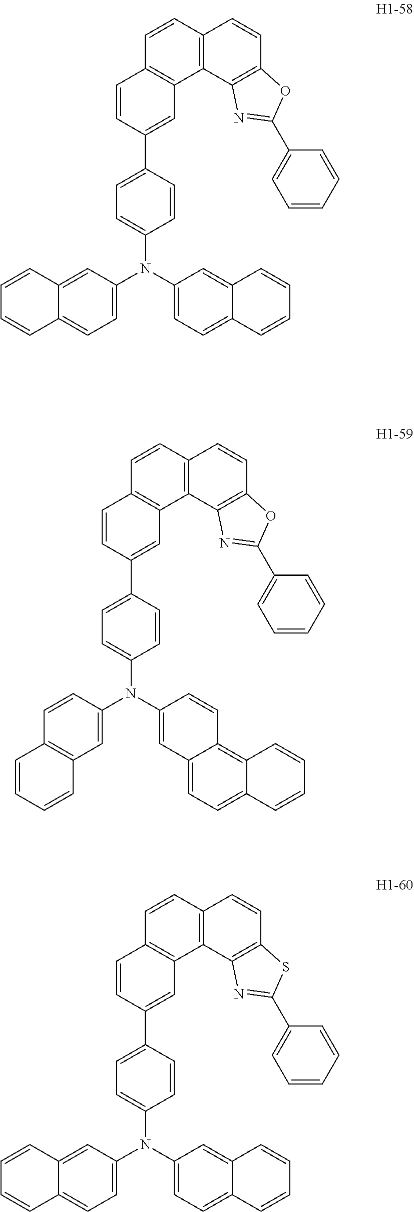 [SMILES: c1ccc(-c2nc3c(ccc4ccc5ccc(-c6ccc(N(c7ccc8ccccc8c7)c7ccc8ccccc8c7)cc6)cc5c43)o2)cc1 ; c1ccc(-c2nc3c(ccc4ccc5ccc(-c6ccc(N(c7ccc8ccccc8c7)c7ccc8c(ccc9ccccc98)c7)cc6)cc5c43)o2)cc1 ; c1ccc(-c2nc3c(ccc4ccc5ccc(-c6ccc(N(c7ccc8ccccc8c7)c7ccc8ccccc8c7)cc6)cc5c43)s2)cc1]

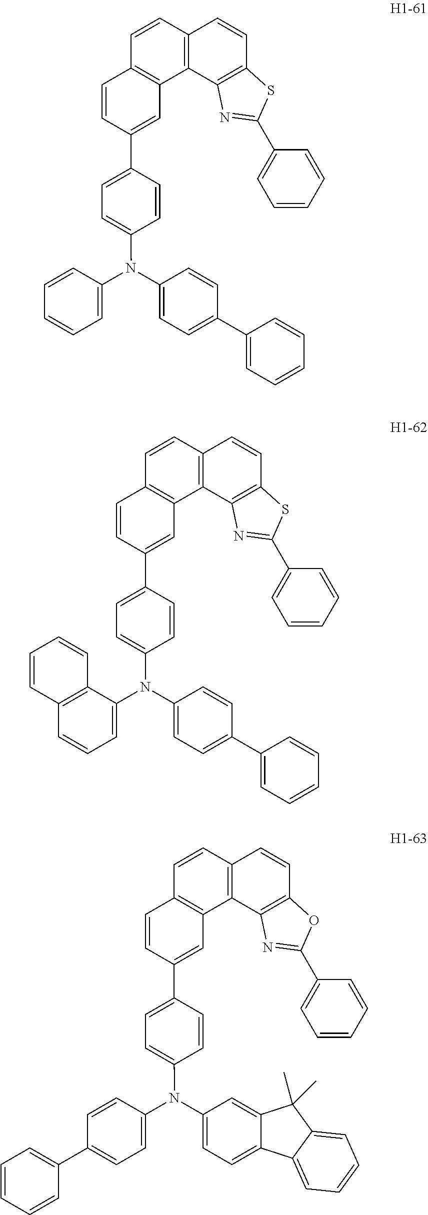 [SMILES: c1ccc(-c2ccc(N(c3ccccc3)c3ccc(-c4ccc5ccc6ccc7sc(-c8ccccc8)nc7c6c5c4)cc3)cc2)cc1 ; c1ccc(-c2ccc(N(c3ccc(-c4ccc5ccc6ccc7sc(-c8ccccc8)nc7c6c5c4)cc3)c3cccc4ccccc34)cc2)cc1 ; CC1(C)c2ccccc2-c2ccc(N(c3ccc(-c4ccccc4)cc3)c3ccc(-c4ccc5ccc6ccc7oc(-c8ccccc8)nc7c6c5c4)cc3)cc21]

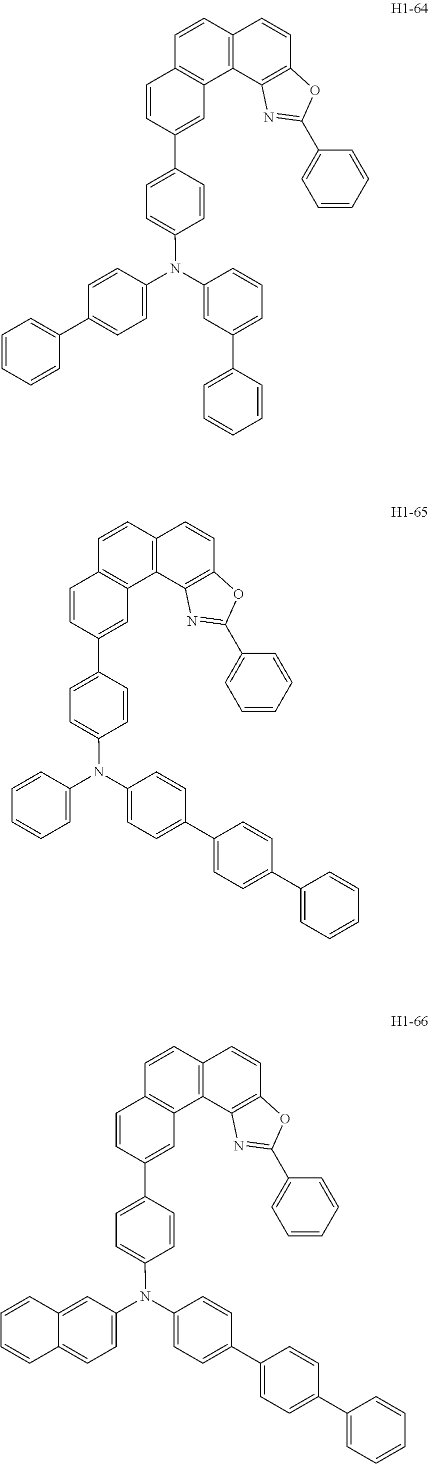 [SMILES: c1ccc(-c2ccc(N(c3ccc(-c4ccc5ccc6ccc7oc(-c8ccccc8)nc7c6c5c4)cc3)c3cccc(-c4ccccc4)c3)cc2)cc1 ; c1ccc(-c2ccc(-c3ccc(N(c4ccccc4)c4ccc(-c5ccc6ccc7ccc8oc(-c9ccccc9)nc8c7c6c5)cc4)cc3)cc2)cc1 ; c1ccc(-c2ccc(-c3ccc(N(c4ccc(-c5ccc6ccc7ccc8oc(-c9ccccc9)nc8c7c6c5)cc4)c4ccc5ccccc5c4)cc3)cc2)cc1]

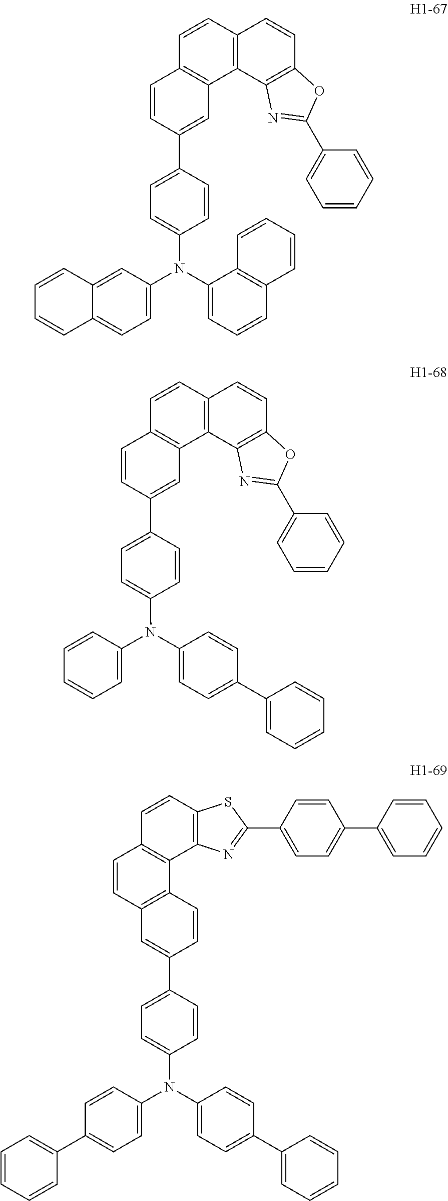 [SMILES: c1ccc(-c2nc3c(ccc4ccc5ccc(-c6ccc(N(c7ccc8ccccc8c7)c7cccc8ccccc78)cc6)cc5c43)o2)cc1 ; c1ccc(-c2ccc(N(c3ccccc3)c3ccc(-c4ccc5ccc6ccc7oc(-c8ccccc8)nc7c6c5c4)cc3)cc2)cc1 ; c1ccc(-c2ccc(-c3nc4c(ccc5ccc6cc(-c7ccc(N(c8ccc(-c9ccccc9)cc8)c8ccc(-c9ccccc9)cc8)cc7)ccc6c54)s3)cc2)cc1]

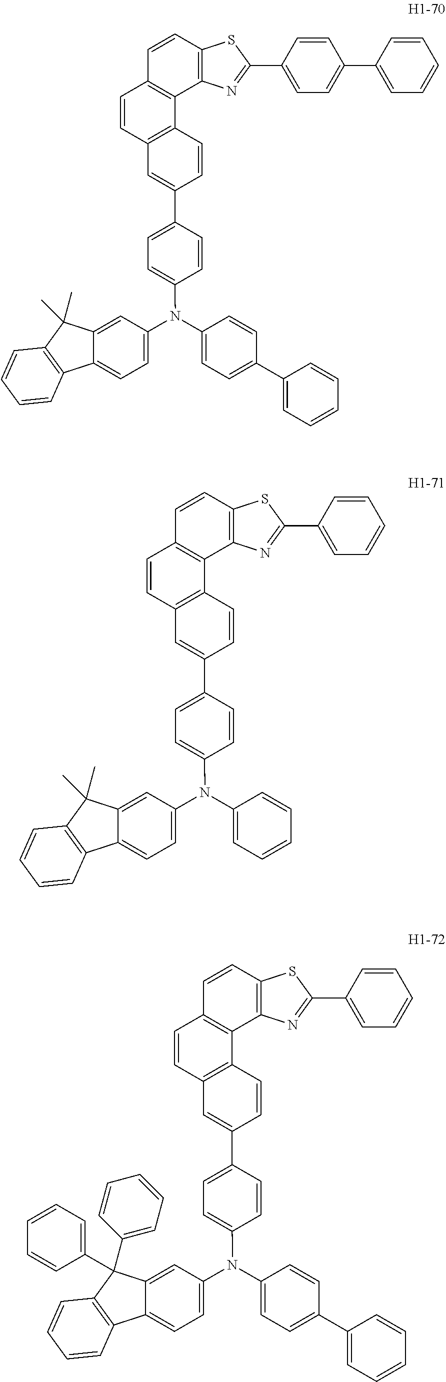 [SMILES: CC1(C)c2ccccc2-c2ccc(N(c3ccc(-c4ccccc4)cc3)c3ccc(-c4ccc5c(ccc6ccc7sc(-c8ccc(-c9ccccc9)cc8)nc7c65)c4)cc3)cc21 ; CC1(C)c2ccccc2-c2ccc(N(c3ccccc3)c3ccc(-c4ccc5c(ccc6ccc7sc(-c8ccccc8)nc7c65)c4)cc3)cc21 ; c1ccc(-c2ccc(N(c3ccc(-c4ccc5c(ccc6ccc7sc(-c8ccccc8)nc7c65)c4)cc3)c3ccc4c(c3)C(c3ccccc3)(c3ccccc3)c3ccccc3-4)cc2)cc1]

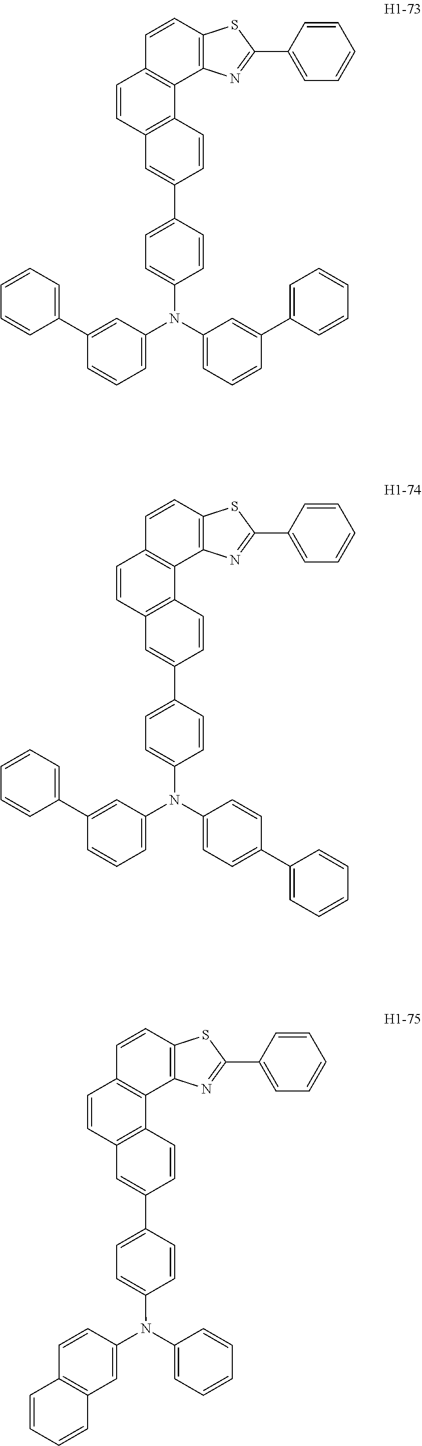 [SMILES: c1ccc(-c2cccc(N(c3ccc(-c4ccc5c(ccc6ccc7sc(-c8ccccc8)nc7c65)c4)cc3)c3cccc(-c4ccccc4)c3)c2)cc1 ; c1ccc(-c2ccc(N(c3ccc(-c4ccc5c(ccc6ccc7sc(-c8ccccc8)nc7c65)c4)cc3)c3cccc(-c4ccccc4)c3)cc2)cc1 ; c1ccc(-c2nc3c(ccc4ccc5cc(-c6ccc(N(c7ccccc7)c7ccc8ccccc8c7)cc6)ccc5c43)s2)cc1]

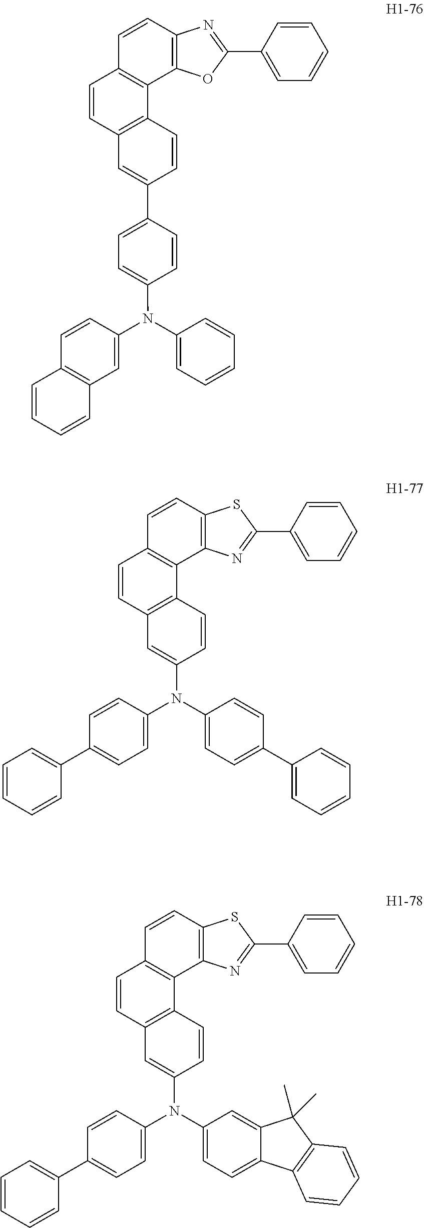 [SMILES: c1ccc(-c2nc3ccc4ccc5cc(-c6ccc(N(c7ccccc7)c7ccc8ccccc8c7)cc6)ccc5c4c3o2)cc1 ; c1ccc(-c2ccc(N(c3ccc(-c4ccccc4)cc3)c3ccc4c(ccc5ccc6sc(-c7ccccc7)nc6c54)c3)cc2)cc1 ; CC1(C)c2ccccc2-c2ccc(N(c3ccc(-c4ccccc4)cc3)c3ccc4c(ccc5ccc6sc(-c7ccccc7)nc6c54)c3)cc21]

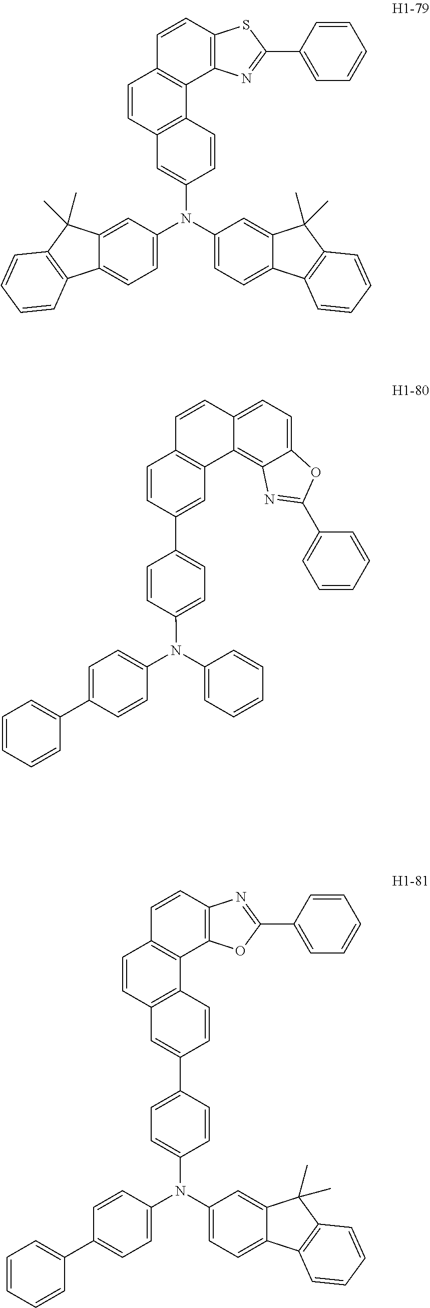 [SMILES: CC1(C)c2ccccc2-c2ccc(N(c3ccc4c(c3)C(C)(C)c3ccccc3-4)c3ccc4c(ccc5ccc6sc(-c7ccccc7)nc6c54)c3)cc21 ; c1ccc(-c2ccc(N(c3ccccc3)c3ccc(-c4ccc5ccc6ccc7oc(-c8ccccc8)nc7c6c5c4)cc3)cc2)cc1 ; CC1(C)c2ccccc2-c2ccc(N(c3ccc(-c4ccccc4)cc3)c3ccc(-c4ccc5c(ccc6ccc7nc(-c8ccccc8)oc7c65)c4)cc3)cc21]

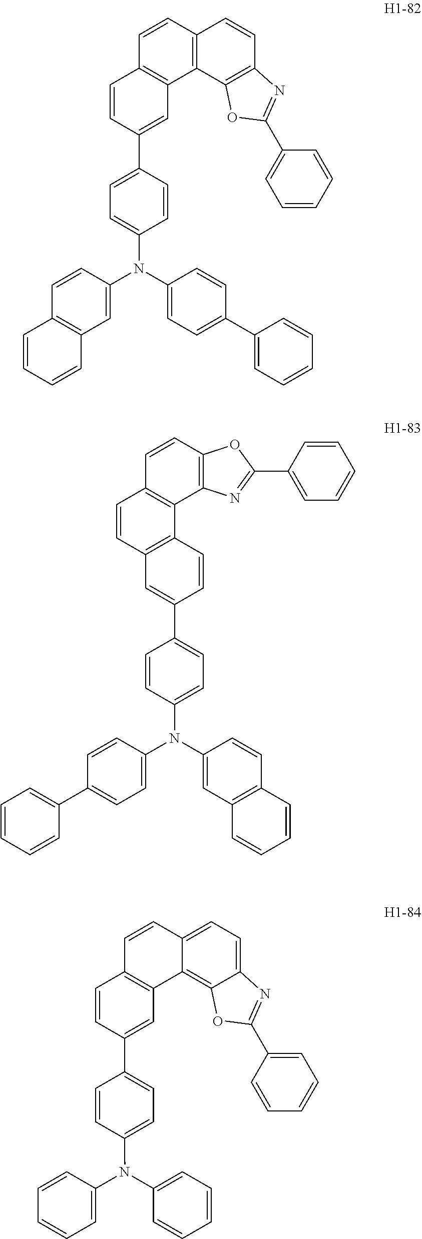 [SMILES: c1ccc(-c2ccc(N(c3ccc(-c4ccc5ccc6ccc7nc(-c8ccccc8)oc7c6c5c4)cc3)c3ccc4ccccc4c3)cc2)cc1 ; c1ccc(-c2ccc(N(c3ccc(-c4ccc5c(ccc6ccc7oc(-c8ccccc8)nc7c65)c4)cc3)c3ccc4ccccc4c3)cc2)cc1 ; c1ccc(-c2nc3ccc4ccc5ccc(-c6ccc(N(c7ccccc7)c7ccccc7)cc6)cc5c4c3o2)cc1]

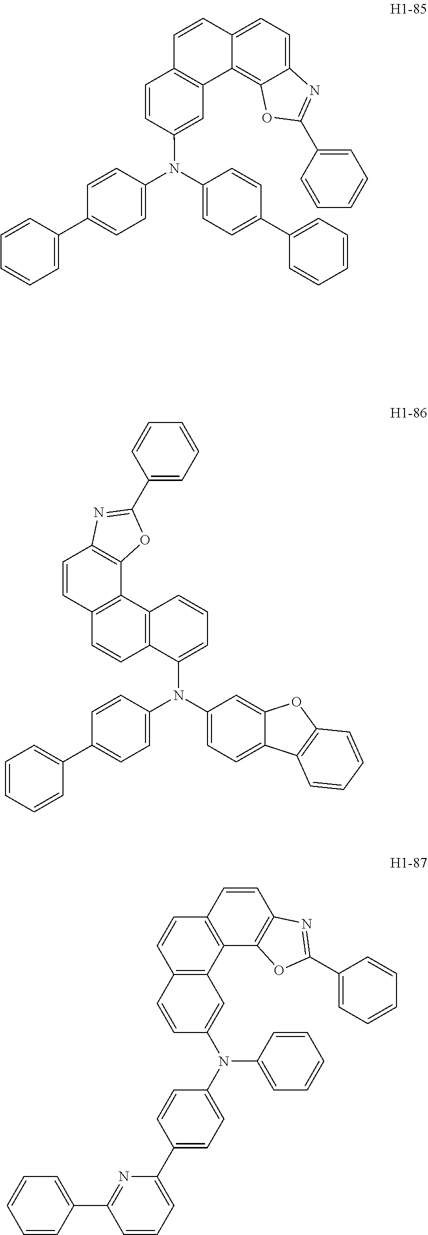 [SMILES: c1ccc(-c2ccc(N(c3ccc(-c4ccccc4)cc3)c3ccc4ccc5ccc6nc(-c7ccccc7)oc6c5c4c3)cc2)cc1 ; c1ccc(-c2ccc(N(c3ccc4c(c3)oc3ccccc34)c3cccc4c3ccc3ccc5nc(-c6ccccc6)oc5c34)cc2)cc1 ; c1ccc(-c2cccc(-c3ccc(N(c4ccccc4)c4ccc5ccc6ccc7nc(-c8ccccc8)oc7c6c5c4)cc3)n2)cc1]

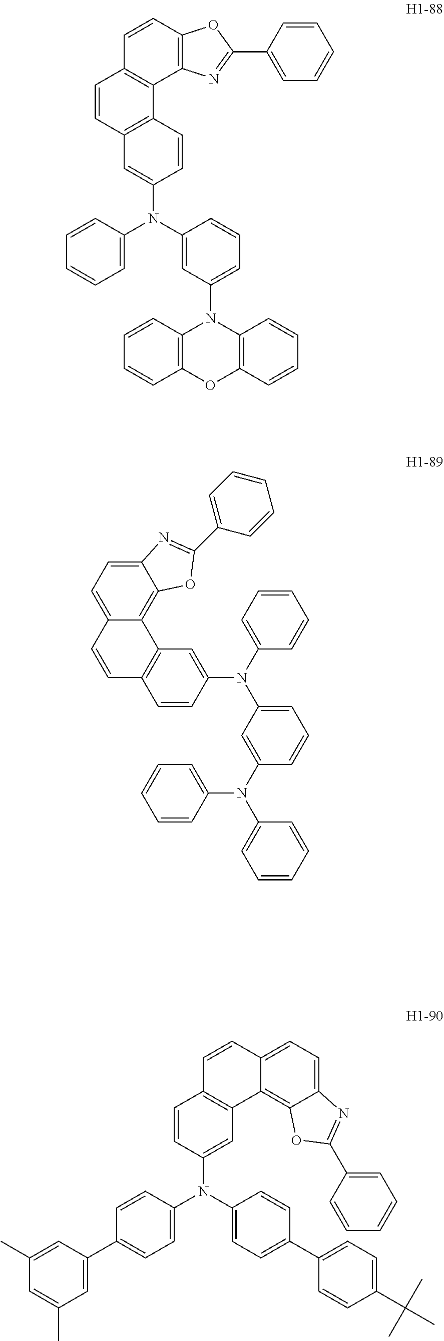 [SMILES: c1ccc(-c2nc3c(ccc4ccc5cc(N(c6ccccc6)c6cccc(N7c8ccccc8Oc8ccccc87)c6)ccc5c43)o2)cc1 ; c1ccc(-c2nc3ccc4ccc5ccc(N(c6ccccc6)c6cccc(N(c7ccccc7)c7ccccc7)c6)cc5c4c3o2)cc1 ; Cc1cc(C)cc(-c2ccc(N(c3ccc(-c4ccc(C(C)(C)C)cc4)cc3)c3ccc4ccc5ccc6nc(-c7ccccc7)oc6c5c4c3)cc2)c1]

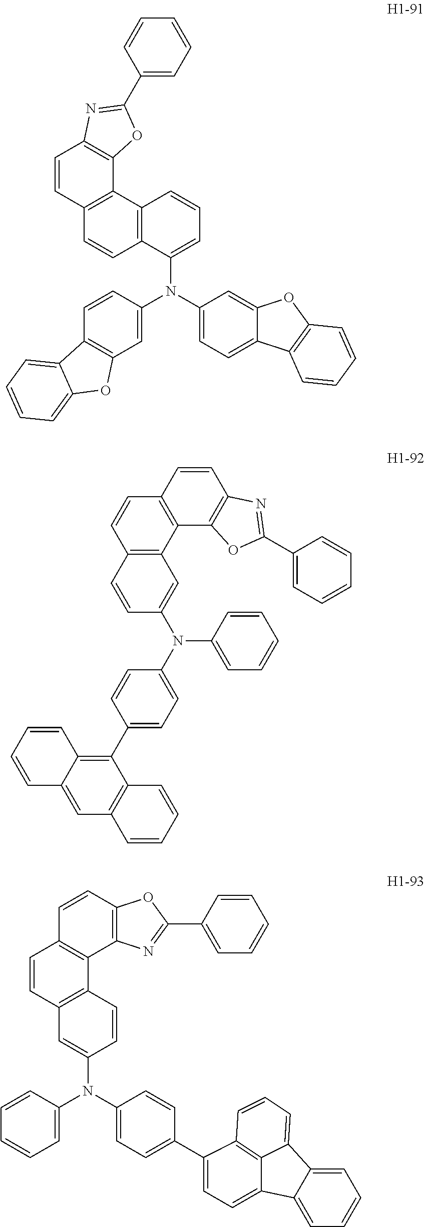 [SMILES: c1ccc(-c2nc3ccc4ccc5c(N(c6ccc7c(c6)oc6ccccc67)c6ccc7c(c6)oc6ccccc67)cccc5c4c3o2)cc1 ; c1ccc(-c2nc3ccc4ccc5ccc(N(c6ccccc6)c6ccc(-c7c8ccccc8cc8ccccc78)cc6)cc5c4c3o2)cc1 ; c1ccc(-c2nc3c(ccc4ccc5cc(N(c6ccccc6)c6ccc(-c7ccc8c9c(cccc79)-c7ccccc7-8)cc6)ccc5c43)o2)cc1]

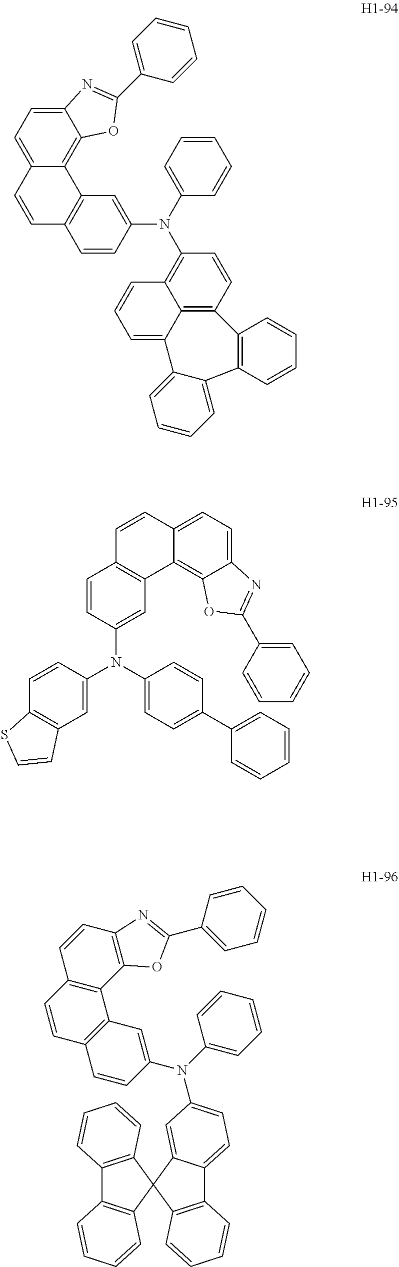 [SMILES: c1ccc(-c2nc3ccc4ccc5ccc(N(c6ccccc6)c6ccc7c8c(cccc68)-c6ccccc6-c6ccccc6-7)cc5c4c3o2)cc1 ; c1ccc(-c2ccc(N(c3ccc4sccc4c3)c3ccc4ccc5ccc6nc(-c7ccccc7)oc6c5c4c3)cc2)cc1 ; c1ccc(-c2nc3ccc4ccc5ccc(N(c6ccccc6)c6ccc7c(c6)C6(c8ccccc8-c8ccccc86)c6ccccc6-7)cc5c4c3o2)cc1]

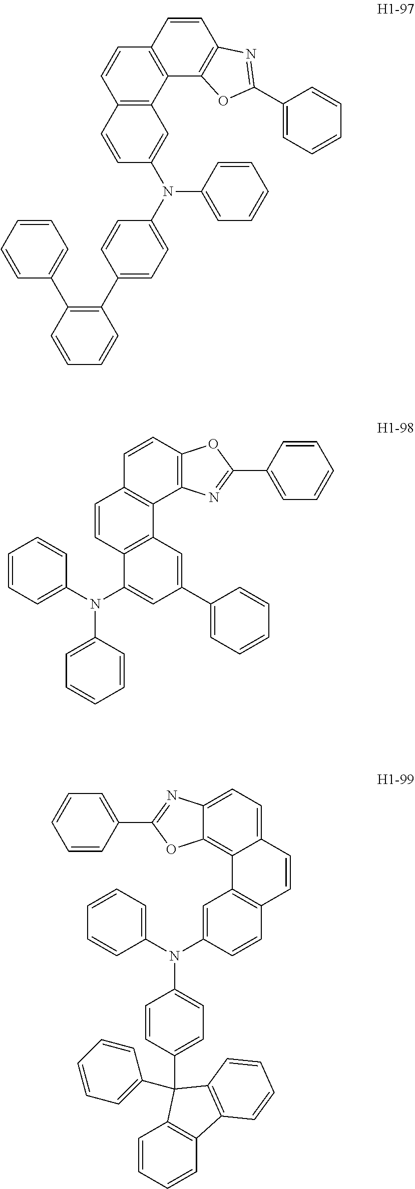 [SMILES: c1ccc(-c2nc3ccc4ccc5ccc(N(c6ccccc6)c6ccc(-c7ccccc7-c7ccccc7)cc6)cc5c4c3o2)cc1 ; c1ccc(-c2cc(N(c3ccccc3)c3ccccc3)c3ccc4ccc5oc(-c6ccccc6)nc5c4c3c2)cc1 ; c1ccc(-c2nc3ccc4ccc5ccc(N(c6ccccc6)c6ccc(C7(c8ccccc8)c8ccccc8-c8ccccc87)cc6)cc5c4c3o2)cc1]

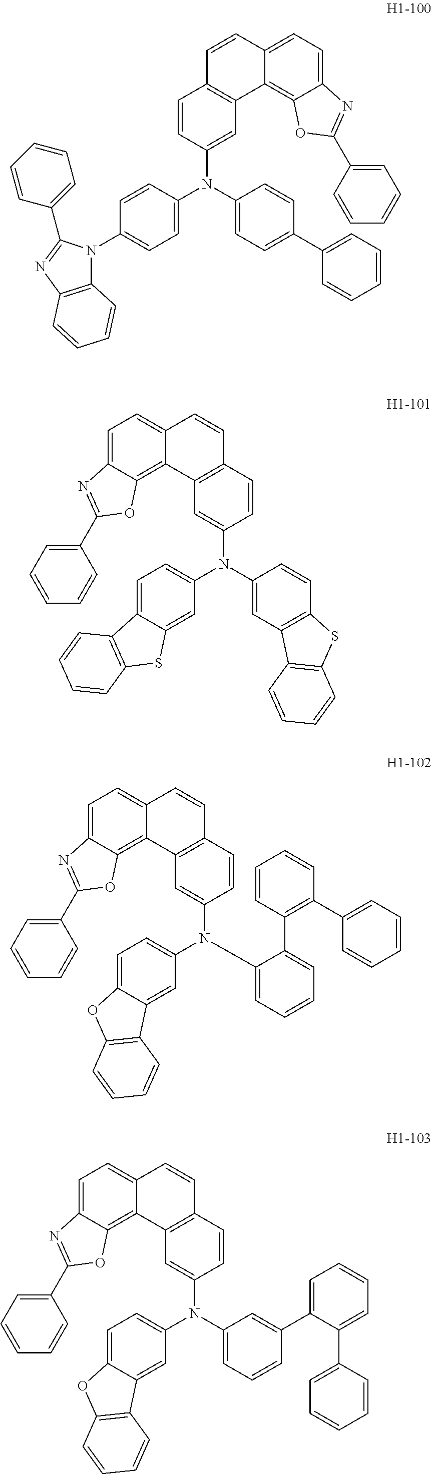 [SMILES: c1ccc(-c2ccc(N(c3ccc(-n4c(-c5ccccc5)nc5ccccc54)cc3)c3ccc4ccc5ccc6nc(-c7ccccc7)oc6c5c4c3)cc2)cc1 ; c1ccc(-c2nc3ccc4ccc5ccc(N(c6ccc7c(c6)sc6ccccc67)c6ccc7sc8ccccc8c7c6)cc5c4c3o2)cc1 ; c1ccc(-c2nc3ccc4ccc5ccc(N(c6ccc7oc8ccccc8c7c6)c6ccccc6-c6ccccc6-c6ccccc6)cc5c4c3o2)cc1 ; c1ccc(-c2nc3ccc4ccc5ccc(N(c6cccc(-c7ccccc7-c7ccccc7)c6)c6ccc7oc8ccccc8c7c6)cc5c4c3o2)cc1]

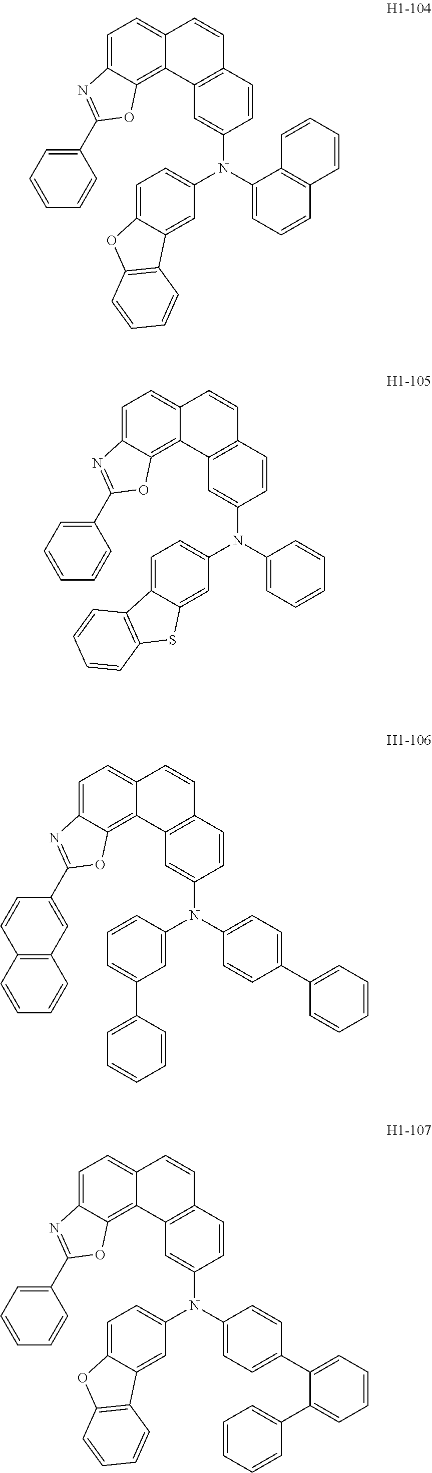 [SMILES: c1ccc(-c2nc3ccc4ccc5ccc(N(c6ccc7oc8ccccc8c7c6)c6cccc7ccccc67)cc5c4c3o2)cc1 ; c1ccc(-c2nc3ccc4ccc5ccc(N(c6ccccc6)c6ccc7c(c6)sc6ccccc67)cc5c4c3o2)cc1 ; c1ccc(-c2ccc(N(c3cccc(-c4ccccc4)c3)c3ccc4ccc5ccc6nc(-c7ccc8ccccc8c7)oc6c5c4c3)cc2)cc1 ; c1ccc(-c2nc3ccc4ccc5ccc(N(c6ccc(-c7ccccc7-c7ccccc7)cc6)c6ccc7oc8ccccc8c7c6)cc5c4c3o2)cc1]

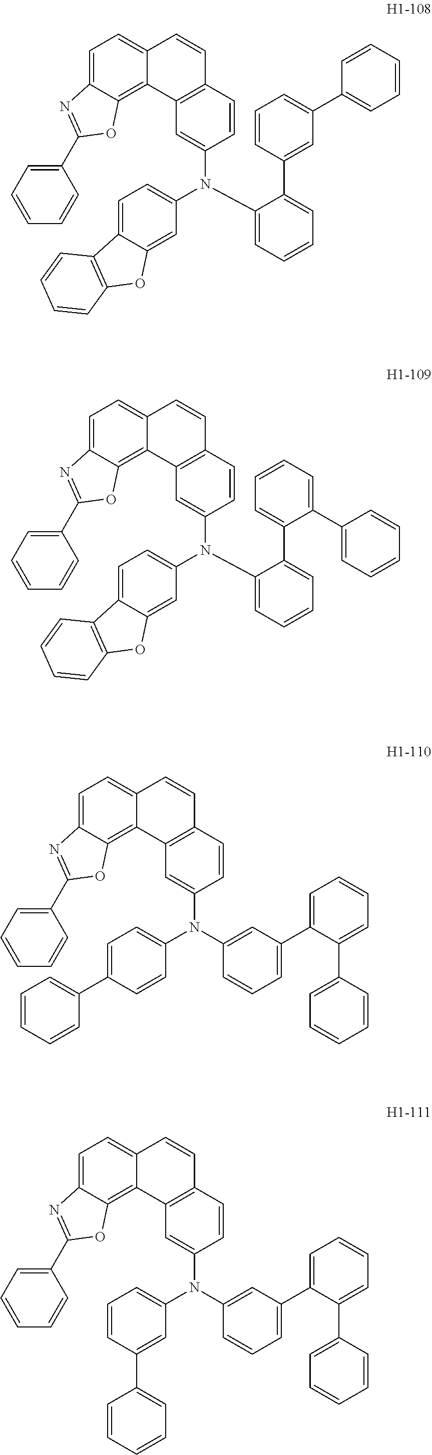 [SMILES: c1ccc(-c2cccc(-c3ccccc3N(c3ccc4c(c3)oc3ccccc34)c3ccc4ccc5ccc6nc(-c7ccccc7)oc6c5c4c3)c2)cc1 ; c1ccc(-c2nc3ccc4ccc5ccc(N(c6ccc7c(c6)oc6ccccc67)c6ccccc6-c6ccccc6-c6ccccc6)cc5c4c3o2)cc1 ; c1ccc(-c2ccc(N(c3cccc(-c4ccccc4-c4ccccc4)c3)c3ccc4ccc5ccc6nc(-c7ccccc7)oc6c5c4c3)cc2)cc1 ; c1ccc(-c2cccc(N(c3cccc(-c4ccccc4-c4ccccc4)c3)c3ccc4ccc5ccc6nc(-c7ccccc7)oc6c5c4c3)c2)cc1]

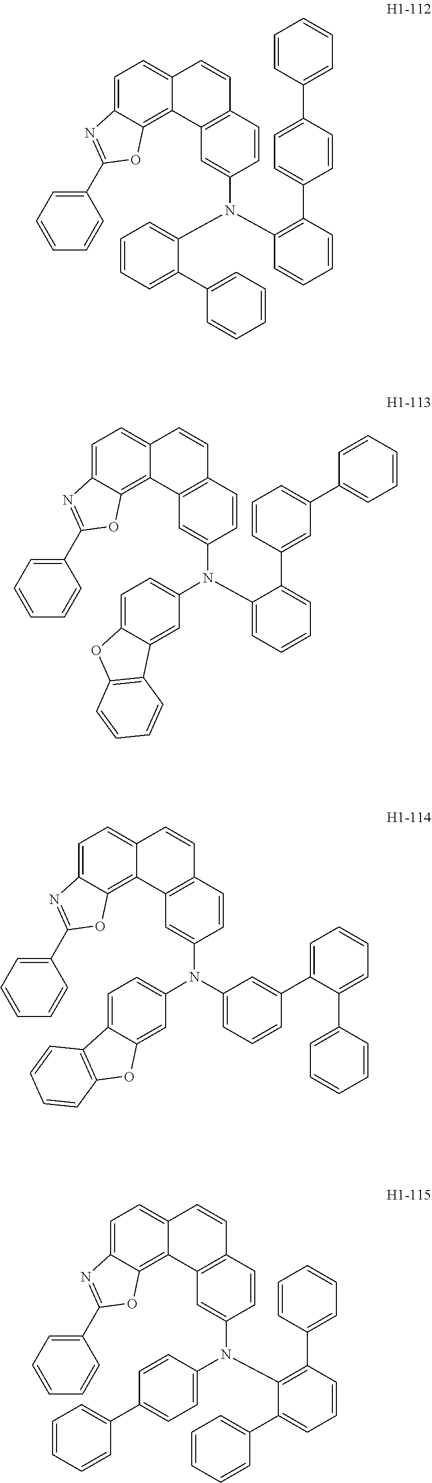 [SMILES: c1ccc(-c2ccc(-c3ccccc3N(c3ccc4ccc5ccc6nc(-c7ccccc7)oc6c5c4c3)c3ccccc3-c3ccccc3)cc2)cc1 ; c1ccc(-c2cccc(-c3ccccc3N(c3ccc4oc5ccccc5c4c3)c3ccc4ccc5ccc6nc(-c7ccccc7)oc6c5c4c3)c2)cc1 ; c1ccc(-c2nc3ccc4ccc5ccc(N(c6cccc(-c7ccccc7-c7ccccc7)c6)c6ccc7c(c6)oc6ccccc67)cc5c4c3o2)cc1 ; c1ccc(-c2ccc(N(c3ccc4ccc5ccc6nc(-c7ccccc7)oc6c5c4c3)c3c(-c4ccccc4)cccc3-c3ccccc3)cc2)cc1]

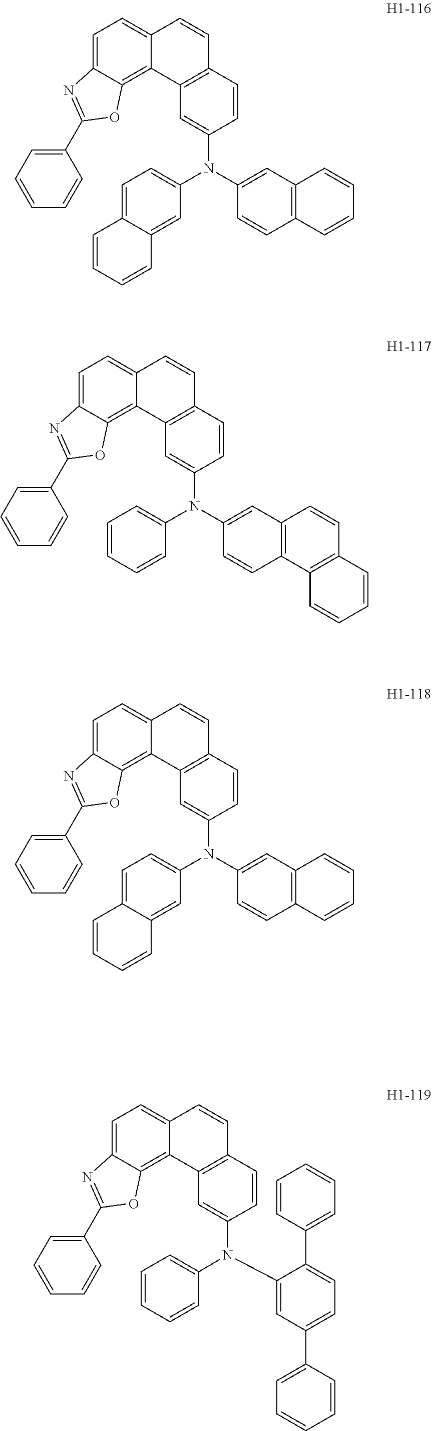 [SMILES: c1ccc(-c2nc3ccc4ccc5ccc(N(c6ccc7ccccc7c6)c6ccc7ccccc7c6)cc5c4c3o2)cc1 ; c1ccc(-c2nc3ccc4ccc5ccc(N(c6ccccc6)c6ccc7c(ccc8ccccc87)c6)cc5c4c3o2)cc1 ; c1ccc(-c2nc3ccc4ccc5ccc(N(c6ccc7ccccc7c6)c6ccc7ccccc7c6)cc5c4c3o2)cc1 ; c1ccc(-c2ccc(-c3ccccc3)c(N(c3ccccc3)c3ccc4ccc5ccc6nc(-c7ccccc7)oc6c5c4c3)c2)cc1]

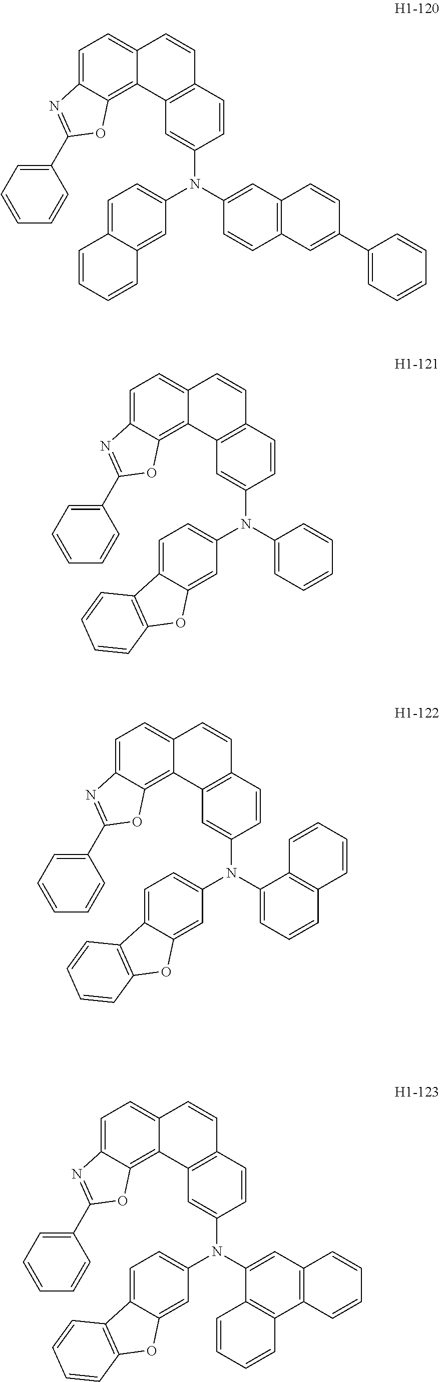 [SMILES: c1ccc(-c2ccc3cc(N(c4ccc5ccccc5c4)c4ccc5ccc6ccc7nc(-c8ccccc8)oc7c6c5c4)ccc3c2)cc1 ; c1ccc(-c2nc3ccc4ccc5ccc(N(c6ccccc6)c6ccc7c(c6)oc6ccccc67)cc5c4c3o2)cc1 ; c1ccc(-c2nc3ccc4ccc5ccc(N(c6ccc7c(c6)oc6ccccc67)c6cccc7ccccc67)cc5c4c3o2)cc1 ; c1ccc(-c2nc3ccc4ccc5ccc(N(c6ccc7c(c6)oc6ccccc67)c6cc7ccccc7c7ccccc67)cc5c4c3o2)cc1]

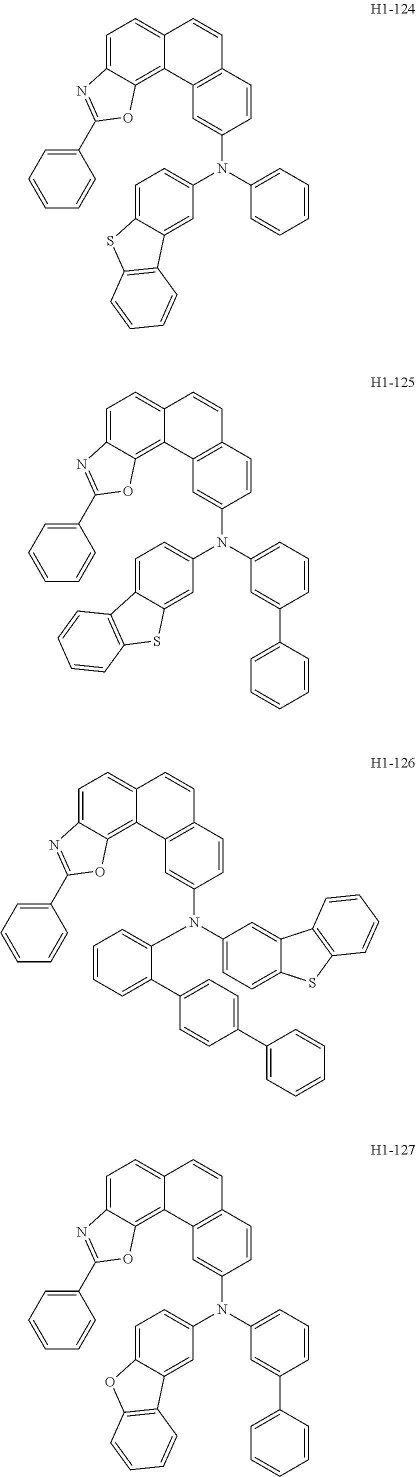 [SMILES: c1ccc(-c2nc3ccc4ccc5ccc(N(c6ccccc6)c6ccc7sc8ccccc8c7c6)cc5c4c3o2)cc1 ; c1ccc(-c2cccc(N(c3ccc4c(c3)sc3ccccc34)c3ccc4ccc5ccc6nc(-c7ccccc7)oc6c5c4c3)c2)cc1 ; c1ccc(-c2ccc(-c3ccccc3N(c3ccc4sc5ccccc5c4c3)c3ccc4ccc5ccc6nc(-c7ccccc7)oc6c5c4c3)cc2)cc1 ; c1ccc(-c2cccc(N(c3ccc4oc5ccccc5c4c3)c3ccc4ccc5ccc6nc(-c7ccccc7)oc6c5c4c3)c2)cc1]

The compound represented by formula 2 may be specifically exemplified by the following compounds, but is not limited thereto.

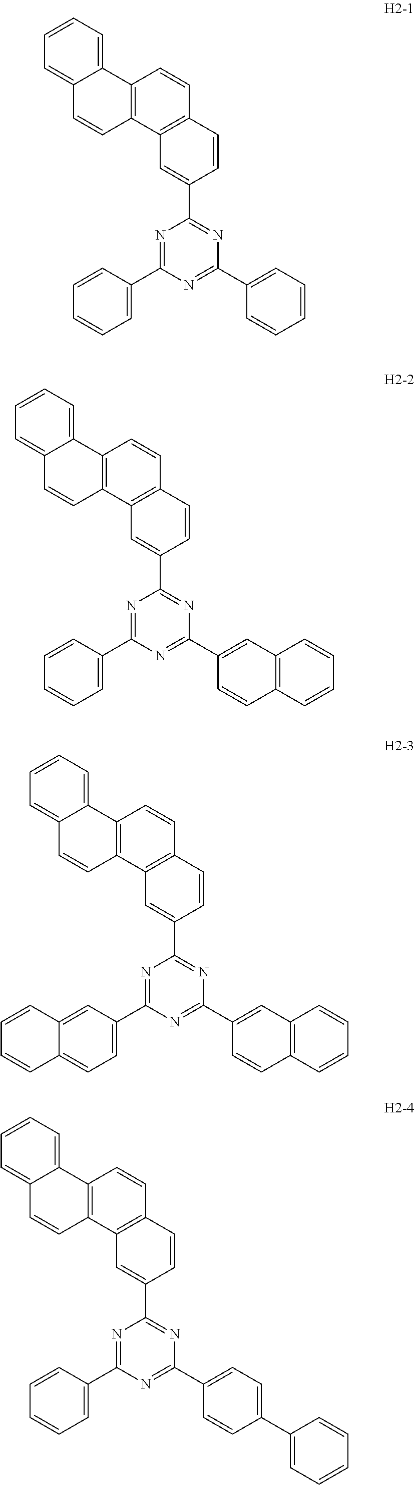

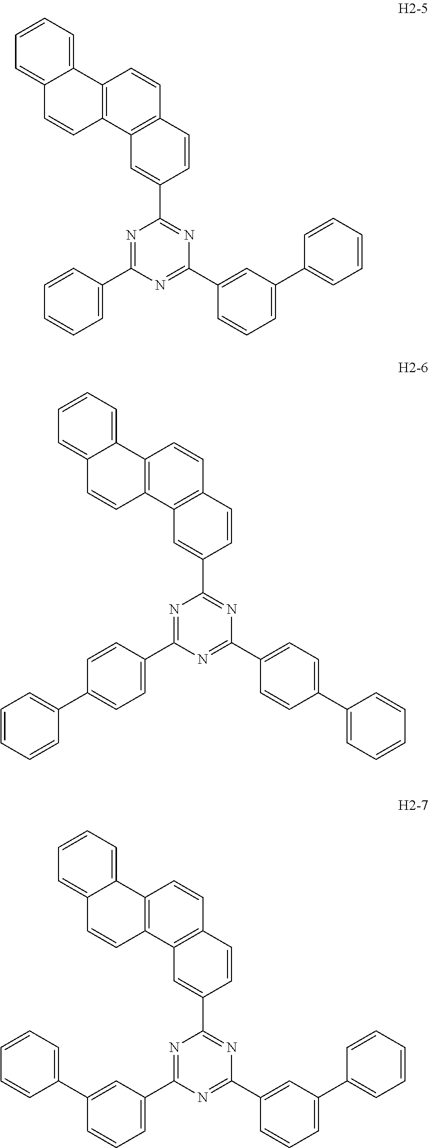 [SMILES: c1ccc(-c2cccc(-c3nc(-c4ccccc4)nc(-c4ccc5ccc6c7ccccc7ccc6c5c4)n3)c2)cc1 ; c1ccc(-c2ccc(-c3nc(-c4ccc(-c5ccccc5)cc4)nc(-c4ccc5ccc6c7ccccc7ccc6c5c4)n3)cc2)cc1 ; c1ccc(-c2cccc(-c3nc(-c4cccc(-c5ccccc5)c4)nc(-c4ccc5ccc6c7ccccc7ccc6c5c4)n3)c2)cc1]

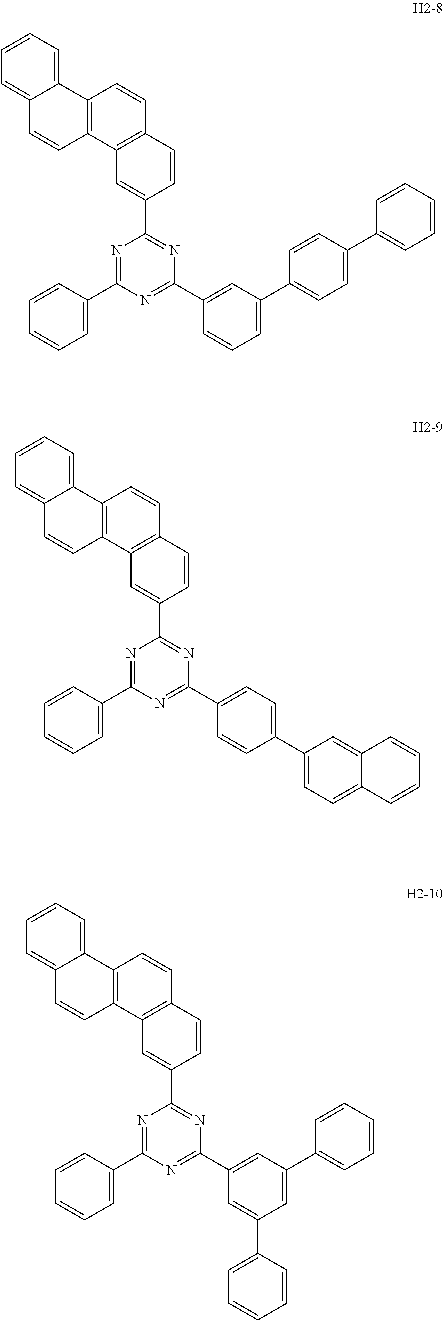 [SMILES: c1ccc(-c2ccc(-c3cccc(-c4nc(-c5ccccc5)nc(-c5ccc6ccc7c8ccccc8ccc7c6c5)n4)c3)cc2)cc1 ; c1ccc(-c2nc(-c3ccc(-c4ccc5ccccc5c4)cc3)nc(-c3ccc4ccc5c6ccccc6ccc5c4c3)n2)cc1 ; c1ccc(-c2cc(-c3ccccc3)cc(-c3nc(-c4ccccc4)nc(-c4ccc5ccc6c7ccccc7ccc6c5c4)n3)c2)cc1]

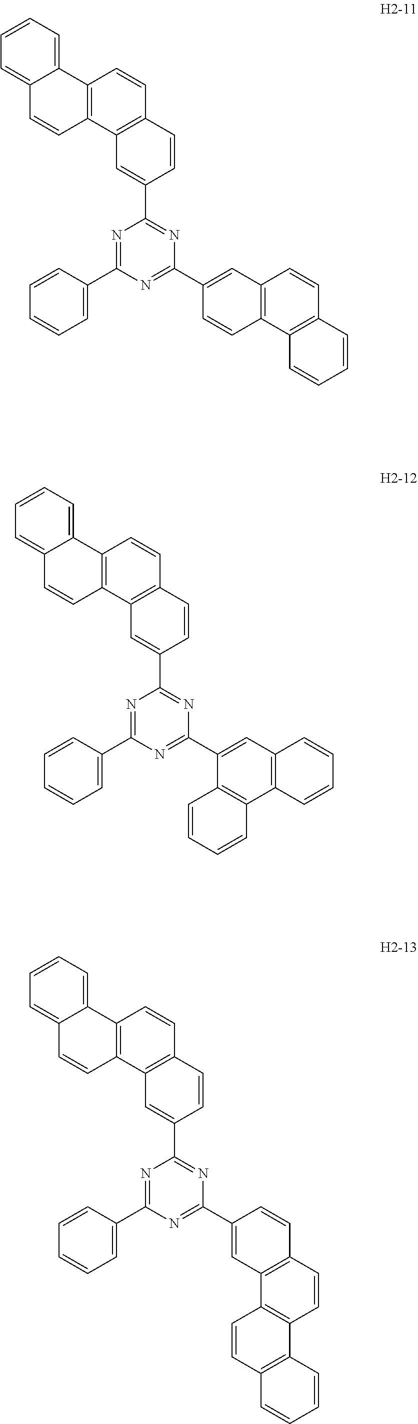 [SMILES: c1ccc(-c2nc(-c3ccc4c(ccc5ccccc54)c3)nc(-c3ccc4ccc5c6ccccc6ccc5c4c3)n2)cc1 ; c1ccc(-c2nc(-c3ccc4ccc5c6ccccc6ccc5c4c3)nc(-c3cc4ccccc4c4ccccc34)n2)cc1 ; c1ccc(-c2nc(-c3ccc4ccc5c6ccccc6ccc5c4c3)nc(-c3ccc4ccc5c6ccccc6ccc5c4c3)n2)cc1]

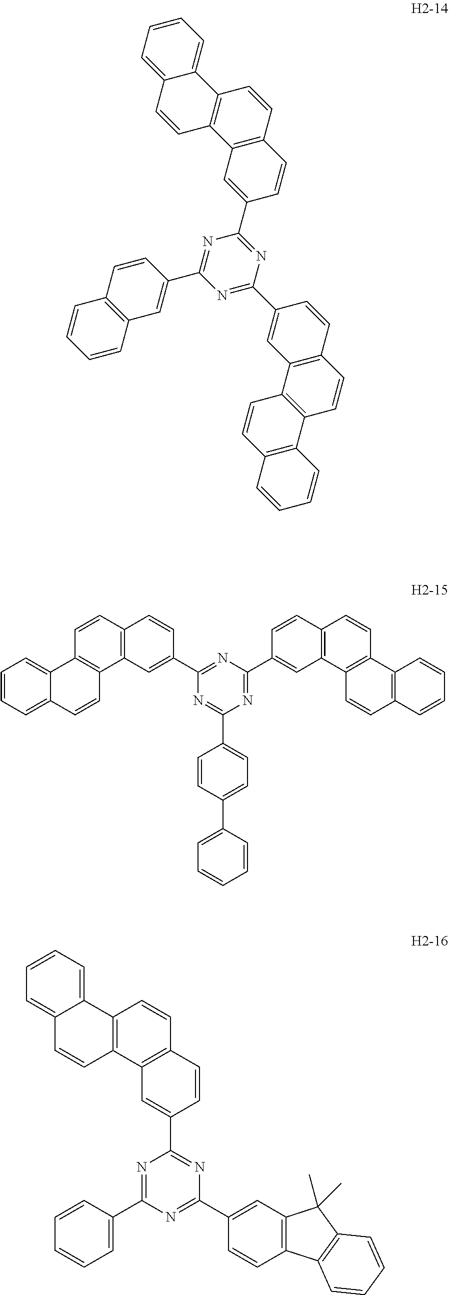 [SMILES: c1ccc2cc(-c3nc(-c4ccc5ccc6c7ccccc7ccc6c5c4)nc(-c4ccc5ccc6c7ccccc7ccc6c5c4)n3)ccc2c1 ; c1ccc(-c2ccc(-c3nc(-c4ccc5ccc6c7ccccc7ccc6c5c4)nc(-c4ccc5ccc6c7ccccc7ccc6c5c4)n3)cc2)cc1 ; CC1(C)c2ccccc2-c2ccc(-c3nc(-c4ccccc4)nc(-c4ccc5ccc6c7ccccc7ccc6c5c4)n3)cc21]

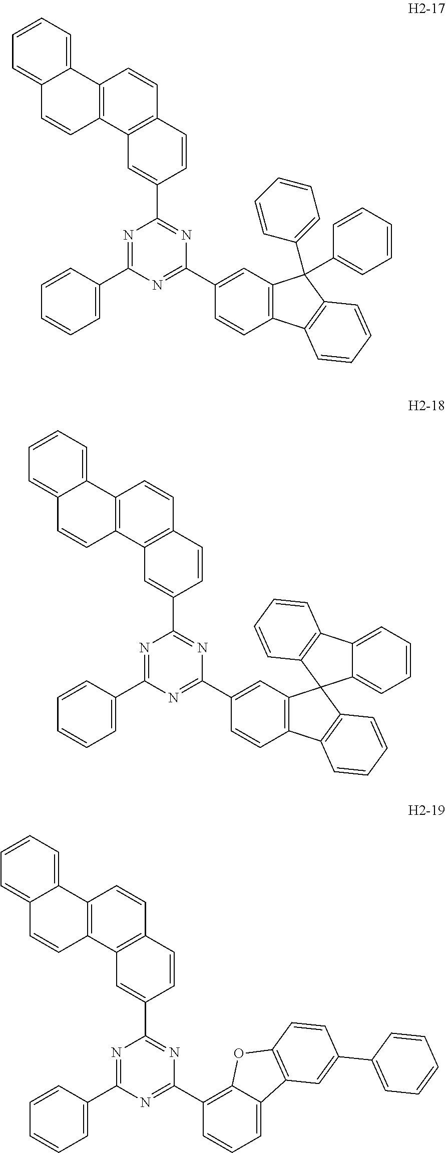 [SMILES: c1ccc(-c2nc(-c3ccc4c(c3)C(c3ccccc3)(c3ccccc3)c3ccccc3-4)nc(-c3ccc4ccc5c6ccccc6ccc5c4c3)n2)cc1 ; c1ccc(-c2nc(-c3ccc4c(c3)C3(c5ccccc5-c5ccccc53)c3ccccc3-4)nc(-c3ccc4ccc5c6ccccc6ccc5c4c3)n2)cc1 ; c1ccc(-c2ccc3oc4c(-c5nc(-c6ccccc6)nc(-c6ccc7ccc8c9ccccc9ccc8c7c6)n5)cccc4c3c2)cc1]

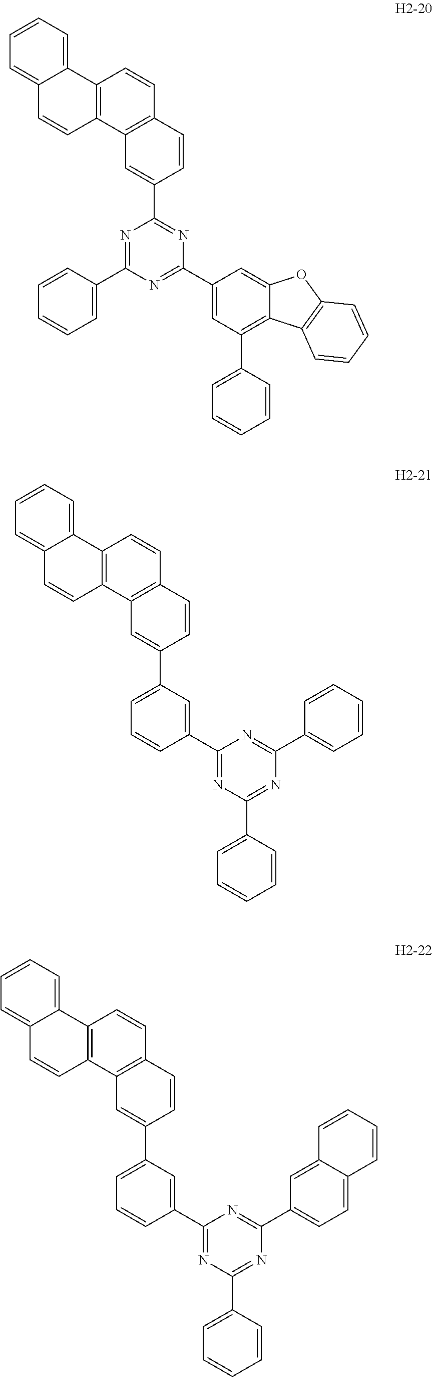 [SMILES: c1ccc(-c2nc(-c3cc(-c4ccccc4)c4c(c3)oc3ccccc34)nc(-c3ccc4ccc5c6ccccc6ccc5c4c3)n2)cc1 ; c1ccc(-c2nc(-c3ccccc3)nc(-c3cccc(-c4ccc5ccc6c7ccccc7ccc6c5c4)c3)n2)cc1 ; c1ccc(-c2nc(-c3cccc(-c4ccc5ccc6c7ccccc7ccc6c5c4)c3)nc(-c3ccc4ccccc4c3)n2)cc1]

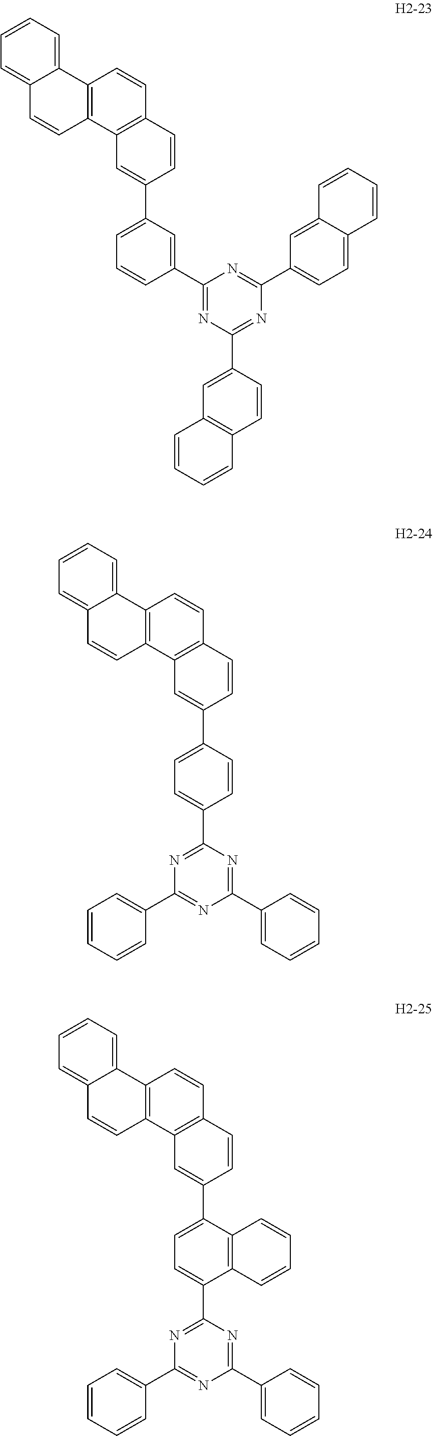 [SMILES: c1cc(-c2ccc3ccc4c5ccccc5ccc4c3c2)cc(-c2nc(-c3ccc4ccccc4c3)nc(-c3ccc4ccccc4c3)n2)c1 ; c1ccc(-c2nc(-c3ccccc3)nc(-c3ccc(-c4ccc5ccc6c7ccccc7ccc6c5c4)cc3)n2)cc1 ; c1ccc(-c2nc(-c3ccccc3)nc(-c3ccc(-c4ccc5ccc6c7ccccc7ccc6c5c4)c4ccccc34)n2)cc1]

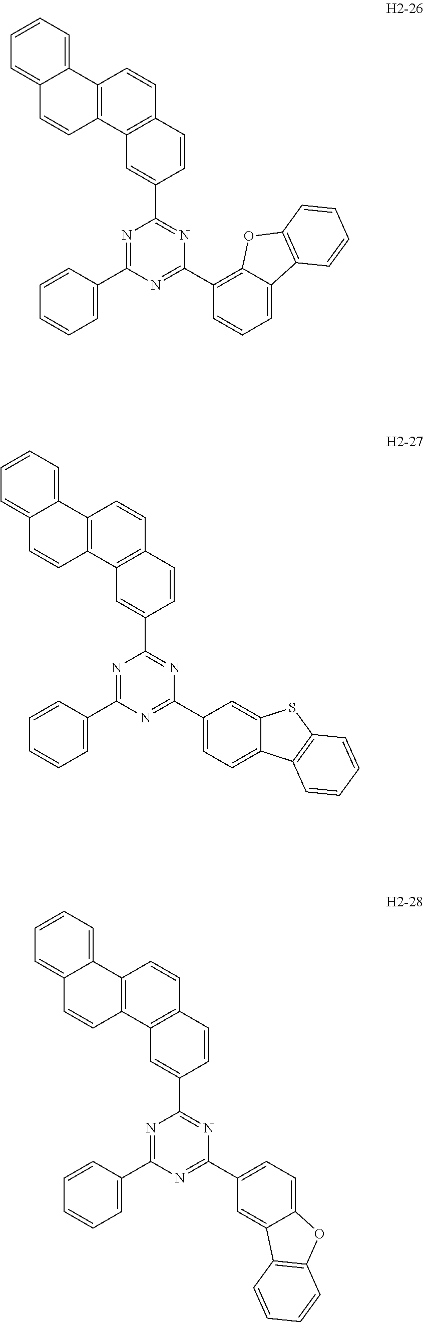 [SMILES: c1ccc(-c2nc(-c3ccc4ccc5c6ccccc6ccc5c4c3)nc(-c3cccc4c3oc3ccccc34)n2)cc1 ; c1ccc(-c2nc(-c3ccc4c(c3)sc3ccccc34)nc(-c3ccc4ccc5c6ccccc6ccc5c4c3)n2)cc1 ; c1ccc(-c2nc(-c3ccc4ccc5c6ccccc6ccc5c4c3)nc(-c3ccc4oc5ccccc5c4c3)n2)cc1]

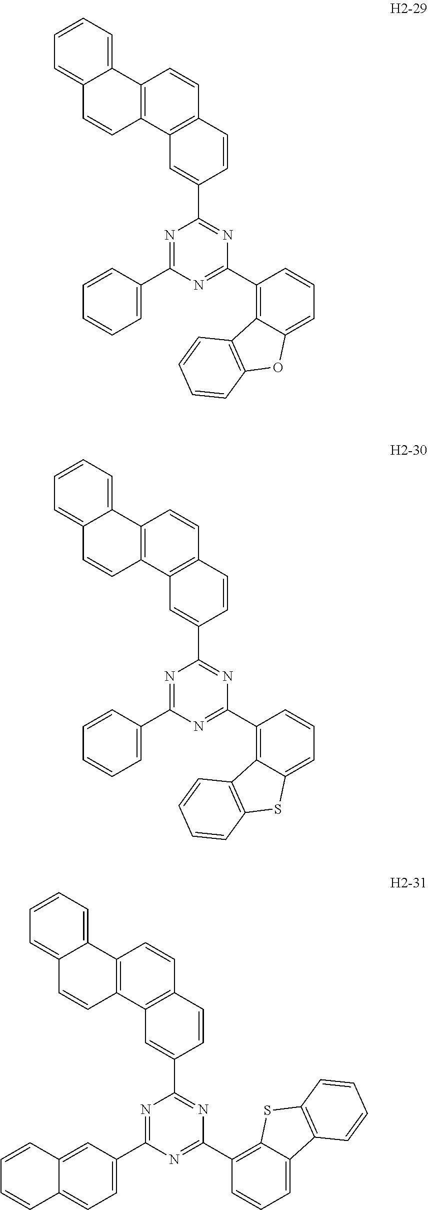 [SMILES: c1ccc(-c2nc(-c3ccc4ccc5c6ccccc6ccc5c4c3)nc(-c3cccc4oc5ccccc5c34)n2)cc1 ; c1ccc(-c2nc(-c3ccc4ccc5c6ccccc6ccc5c4c3)nc(-c3cccc4sc5ccccc5c34)n2)cc1 ; c1ccc2cc(-c3nc(-c4ccc5ccc6c7ccccc7ccc6c5c4)nc(-c4cccc5c4sc4ccccc45)n3)ccc2c1]

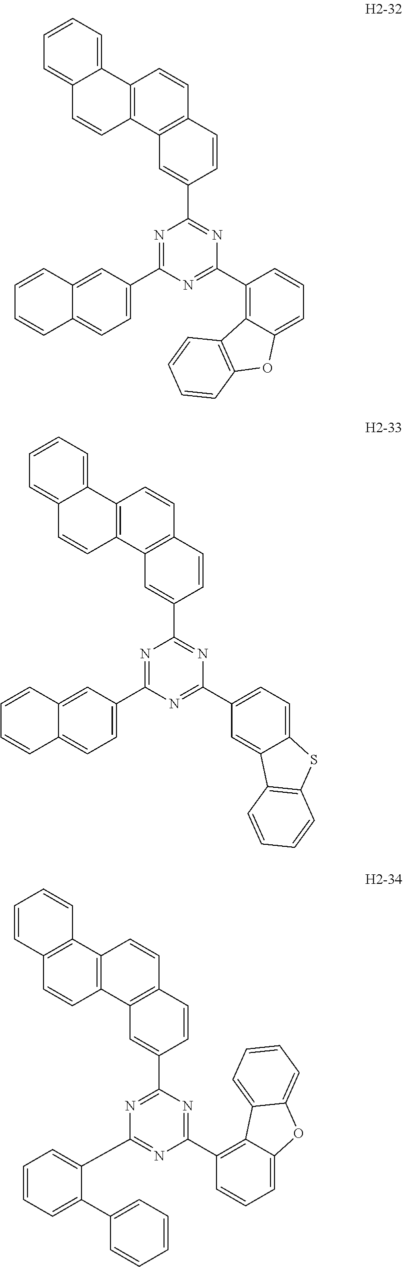 [SMILES: c1ccc2cc(-c3nc(-c4ccc5ccc6c7ccccc7ccc6c5c4)nc(-c4cccc5oc6ccccc6c45)n3)ccc2c1 ; c1ccc2cc(-c3nc(-c4ccc5ccc6c7ccccc7ccc6c5c4)nc(-c4ccc5sc6ccccc6c5c4)n3)ccc2c1 ; c1ccc(-c2ccccc2-c2nc(-c3ccc4ccc5c6ccccc6ccc5c4c3)nc(-c3cccc4oc5ccccc5c34)n2)cc1]

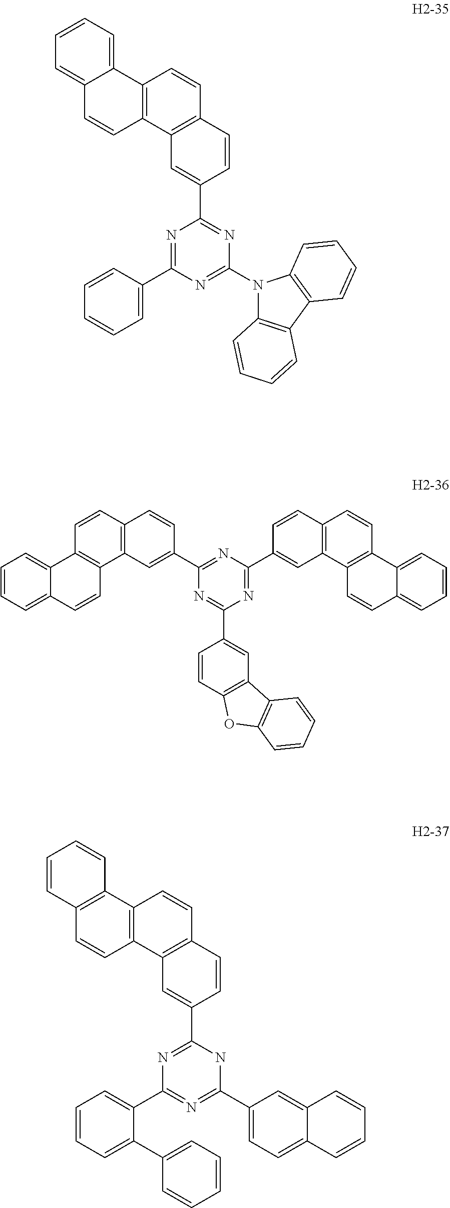

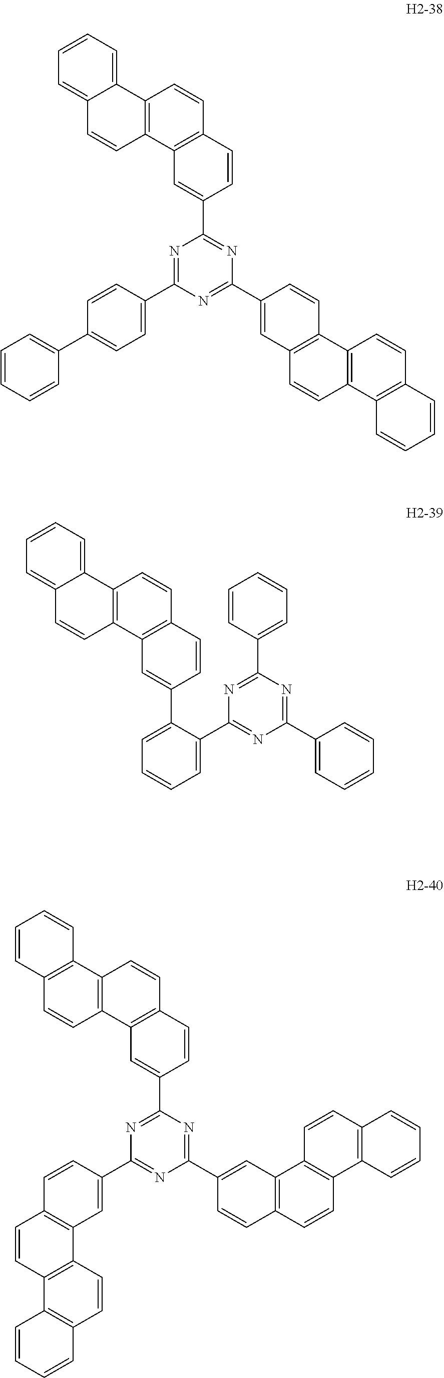 [SMILES: c1ccc(-c2ccc(-c3nc(-c4ccc5c(ccc6c7ccccc7ccc56)c4)nc(-c4ccc5ccc6c7ccccc7ccc6c5c4)n3)cc2)cc1 ; c1ccc(-c2nc(-c3ccccc3)nc(-c3ccccc3-c3ccc4ccc5c6ccccc6ccc5c4c3)n2)cc1 ; c1ccc2c(c1)ccc1c3cc(-c4nc(-c5ccc6ccc7c8ccccc8ccc7c6c5)nc(-c5ccc6ccc7c8ccccc8ccc7c6c5)n4)ccc3ccc21]

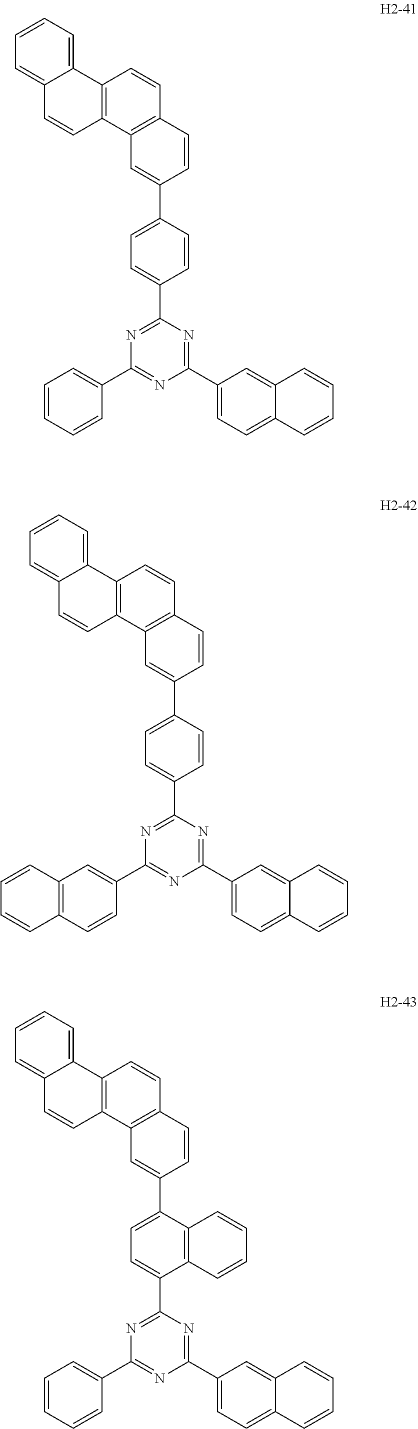 [SMILES: c1ccc(-c2nc(-c3ccc(-c4ccc5ccc6c7ccccc7ccc6c5c4)cc3)nc(-c3ccc4ccccc4c3)n2)cc1 ; c1ccc2cc(-c3nc(-c4ccc(-c5ccc6ccc7c8ccccc8ccc7c6c5)cc4)nc(-c4ccc5ccccc5c4)n3)ccc2c1 ; c1ccc(-c2nc(-c3ccc4ccccc4c3)nc(-c3ccc(-c4ccc5ccc6c7ccccc7ccc6c5c4)c4ccccc34)n2)cc1]

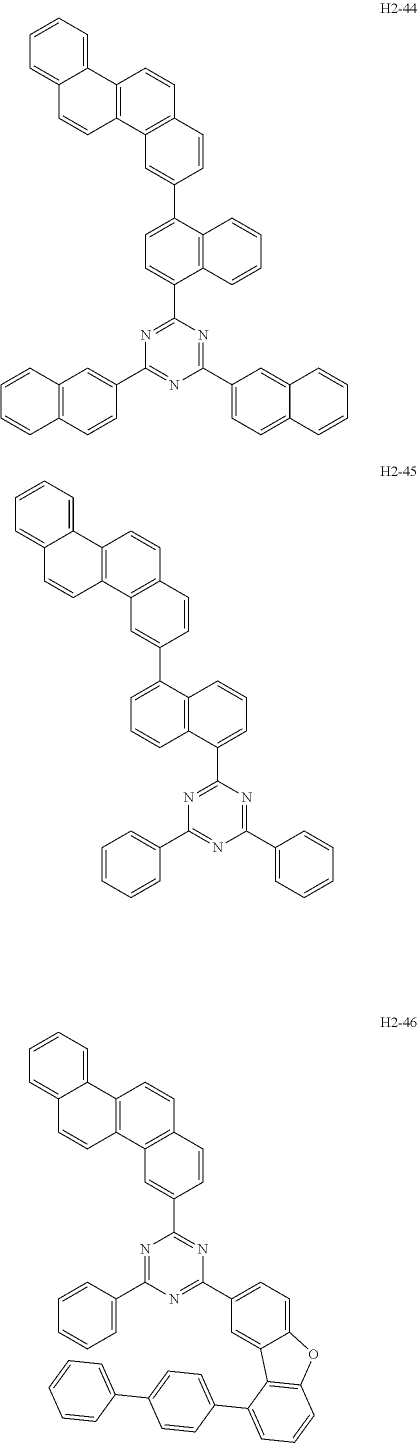 [SMILES: c1ccc2cc(-c3nc(-c4ccc5ccccc5c4)nc(-c4ccc(-c5ccc6ccc7c8ccccc8ccc7c6c5)c5ccccc45)n3)ccc2c1 ; c1ccc(-c2nc(-c3ccccc3)nc(-c3cccc4c(-c5ccc6ccc7c8ccccc8ccc7c6c5)cccc34)n2)cc1 ; c1ccc(-c2ccc(-c3cccc4oc5ccc(-c6nc(-c7ccccc7)nc(-c7ccc8ccc9c%10ccccc%10ccc9c8c7)n6)cc5c34)cc2)cc1]

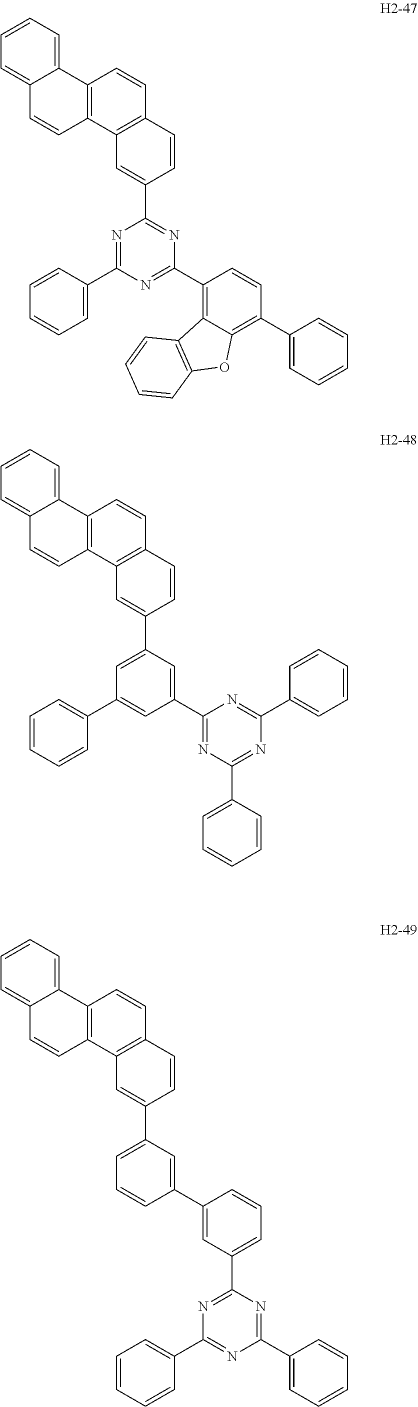 [SMILES: c1ccc(-c2nc(-c3ccc4ccc5c6ccccc6ccc5c4c3)nc(-c3ccc(-c4ccccc4)c4oc5ccccc5c34)n2)cc1 ; c1ccc(-c2cc(-c3ccc4ccc5c6ccccc6ccc5c4c3)cc(-c3nc(-c4ccccc4)nc(-c4ccccc4)n3)c2)cc1 ; c1ccc(-c2nc(-c3ccccc3)nc(-c3cccc(-c4cccc(-c5ccc6ccc7c8ccccc8ccc7c6c5)c4)c3)n2)cc1]

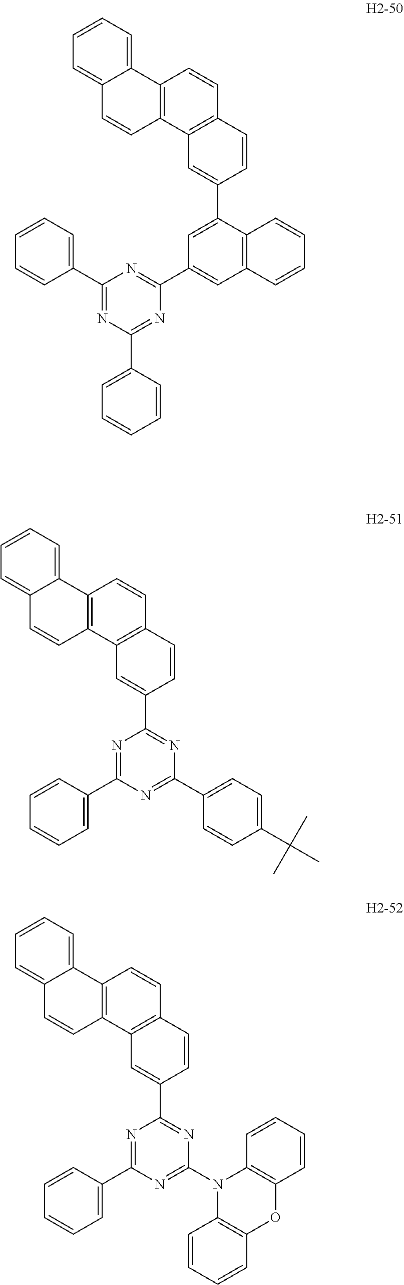 [SMILES: c1ccc(-c2nc(-c3ccccc3)nc(-c3cc(-c4ccc5ccc6c7ccccc7ccc6c5c4)c4ccccc4c3)n2)cc1 ; CC(C)(C)c1ccc(-c2nc(-c3ccccc3)nc(-c3ccc4ccc5c6ccccc6ccc5c4c3)n2)cc1 ; c1ccc(-c2nc(-c3ccc4ccc5c6ccccc6ccc5c4c3)nc(N3c4ccccc4Oc4ccccc43)n2)cc1]

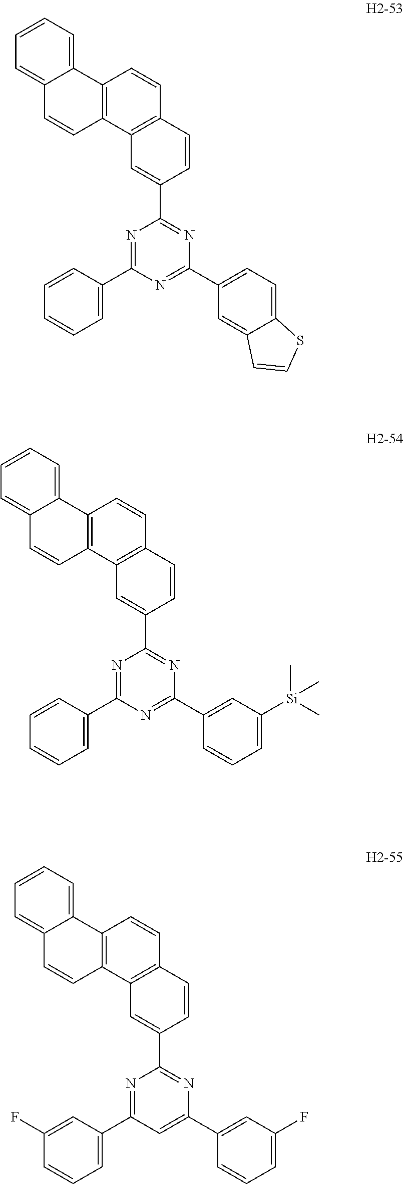 [SMILES: c1ccc(-c2nc(-c3ccc4sccc4c3)nc(-c3ccc4ccc5c6ccccc6ccc5c4c3)n2)cc1 ; C[Si](C)(C)c1cccc(-c2nc(-c3ccccc3)nc(-c3ccc4ccc5c6ccccc6ccc5c4c3)n2)c1 ; Fc1cccc(-c2cc(-c3cccc(F)c3)nc(-c3ccc4ccc5c6ccccc6ccc5c4c3)n2)c1]

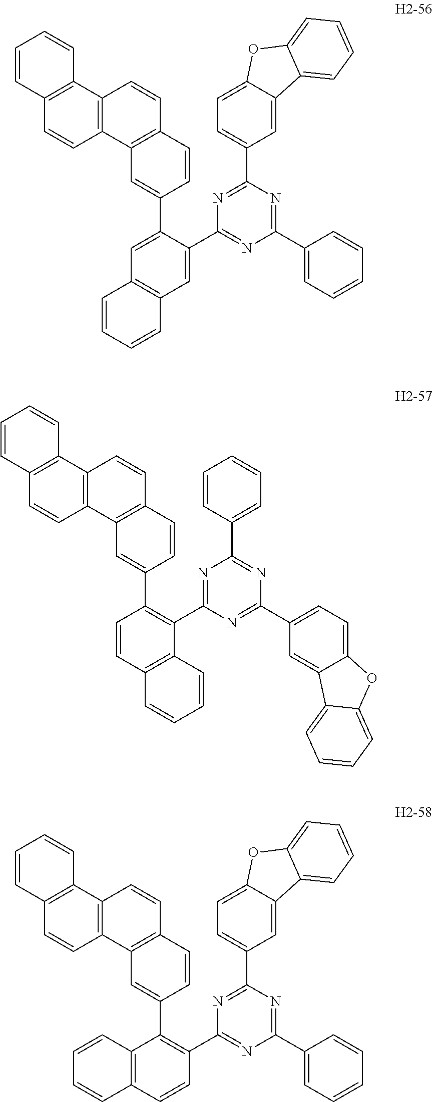 [SMILES: c1ccc(-c2nc(-c3ccc4oc5ccccc5c4c3)nc(-c3cc4ccccc4cc3-c3ccc4ccc5c6ccccc6ccc5c4c3)n2)cc1 ; c1ccc(-c2nc(-c3ccc4oc5ccccc5c4c3)nc(-c3c(-c4ccc5ccc6c7ccccc7ccc6c5c4)ccc4ccccc34)n2)cc1 ; c1ccc(-c2nc(-c3ccc4oc5ccccc5c4c3)nc(-c3ccc4ccccc4c3-c3ccc4ccc5c6ccccc6ccc5c4c3)n2)cc1]

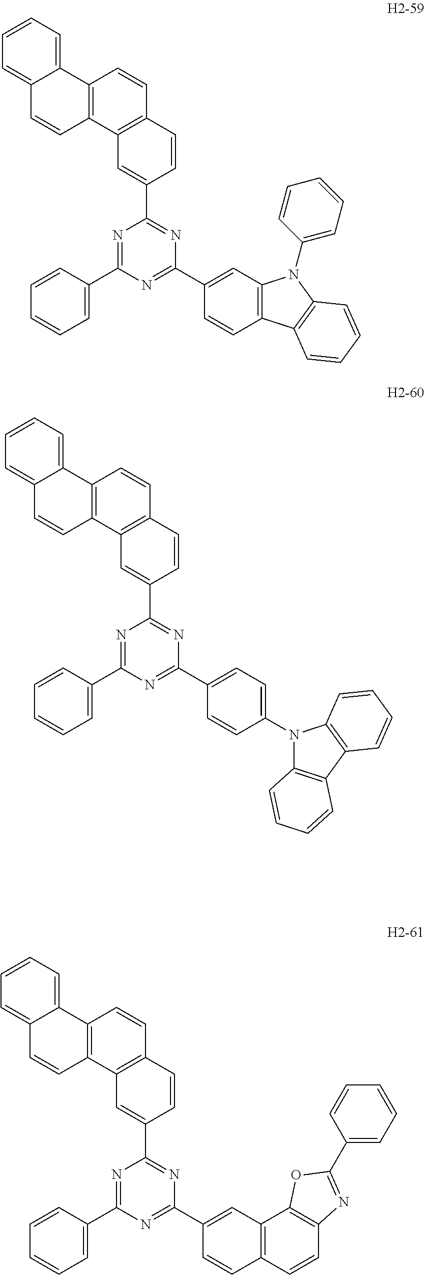 [SMILES: c1ccc(-c2nc(-c3ccc4ccc5c6ccccc6ccc5c4c3)nc(-c3ccc4c5ccccc5n(-c5ccccc5)c4c3)n2)cc1 ; c1ccc(-c2nc(-c3ccc(-n4c5ccccc5c5ccccc54)cc3)nc(-c3ccc4ccc5c6ccccc6ccc5c4c3)n2)cc1 ; c1ccc(-c2nc(-c3ccc4ccc5c6ccccc6ccc5c4c3)nc(-c3ccc4ccc5nc(-c6ccccc6)oc5c4c3)n2)cc1]

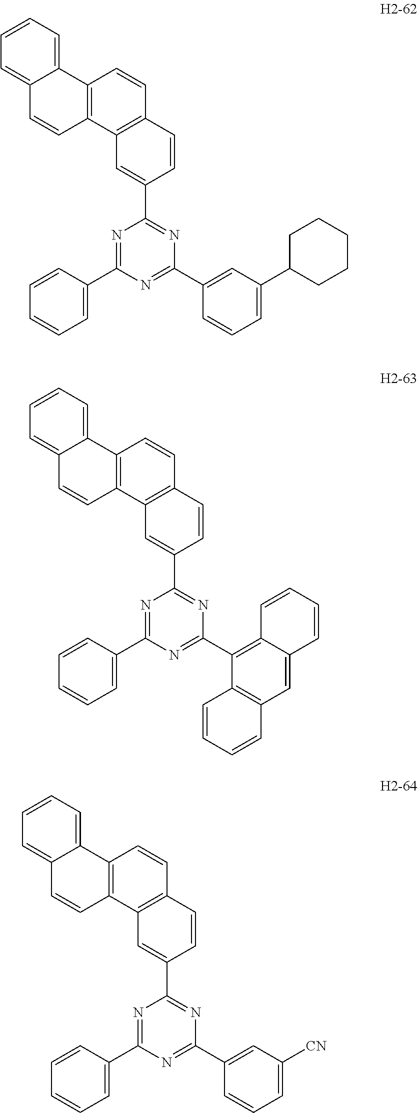 [SMILES: c1ccc(-c2nc(-c3cccc(C4CCCCC4)c3)nc(-c3ccc4ccc5c6ccccc6ccc5c4c3)n2)cc1 ; c1ccc(-c2nc(-c3ccc4ccc5c6ccccc6ccc5c4c3)nc(-c3c4ccccc4cc4ccccc34)n2)cc1 ; N#Cc1cccc(-c2nc(-c3ccccc3)nc(-c3ccc4ccc5c6ccccc6ccc5c4c3)n2)c1]

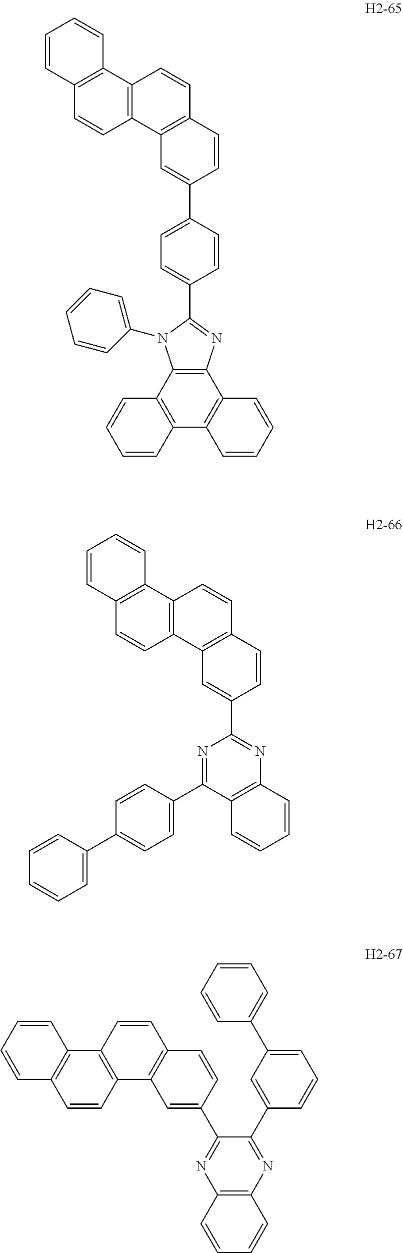 [SMILES: c1ccc(-n2c(-c3ccc(-c4ccc5ccc6c7ccccc7ccc6c5c4)cc3)nc3c4ccccc4c4ccccc4c32)cc1 ; c1ccc(-c2ccc(-c3nc(-c4ccc5ccc6c7ccccc7ccc6c5c4)nc4ccccc34)cc2)cc1 ; c1ccc(-c2cccc(-c3nc4ccccc4nc3-c3ccc4ccc5c6ccccc6ccc5c4c3)c2)cc1]

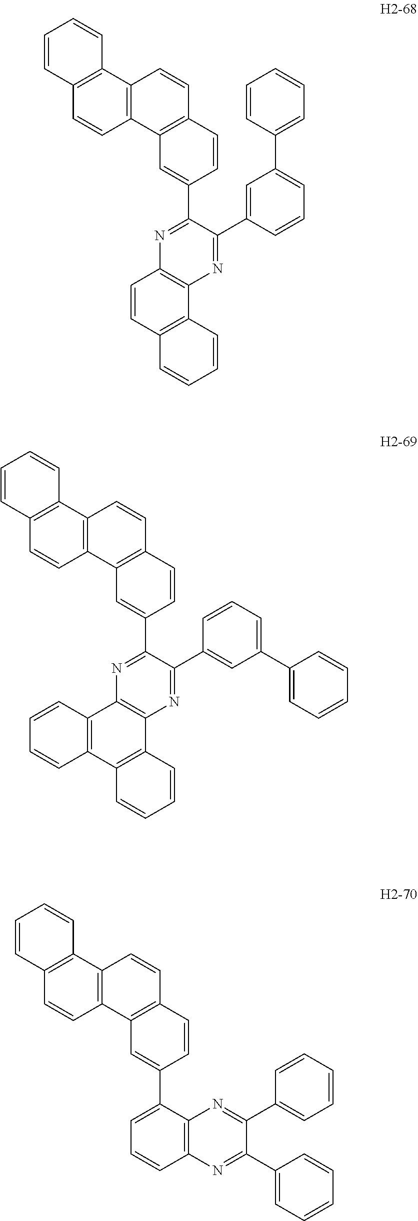 [SMILES: c1ccc(-c2cccc(-c3nc4c(ccc5ccccc54)nc3-c3ccc4ccc5c6ccccc6ccc5c4c3)c2)cc1 ; c1ccc(-c2cccc(-c3nc4c5ccccc5c5ccccc5c4nc3-c3ccc4ccc5c6ccccc6ccc5c4c3)c2)cc1 ; c1ccc(-c2nc3cccc(-c4ccc5ccc6c7ccccc7ccc6c5c4)c3nc2-c2ccccc2)cc1]

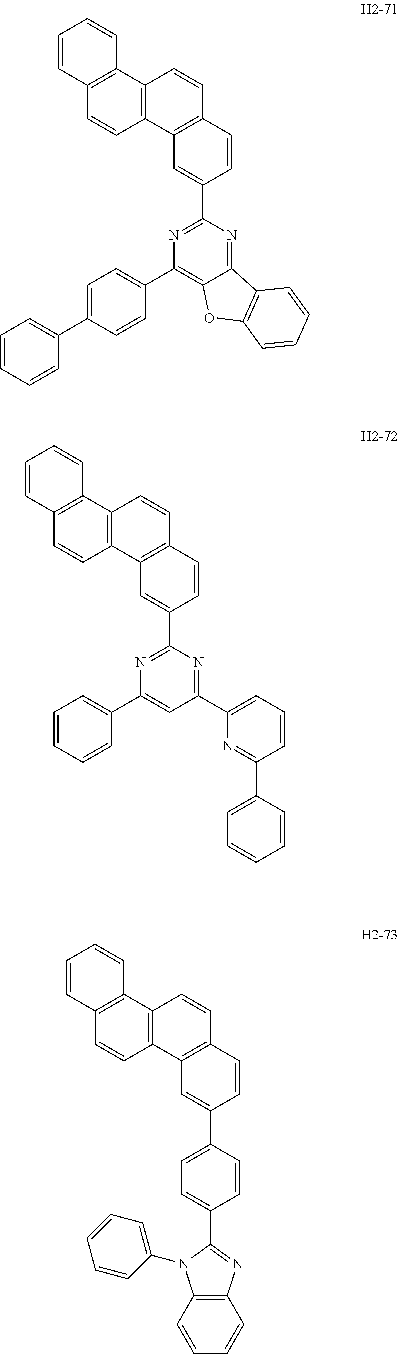 [SMILES: c1ccc(-c2ccc(-c3nc(-c4ccc5ccc6c7ccccc7ccc6c5c4)nc4c3oc3ccccc34)cc2)cc1 ; c1ccc(-c2cccc(-c3cc(-c4ccccc4)nc(-c4ccc5ccc6c7ccccc7ccc6c5c4)n3)n2)cc1 ; c1ccc(-n2c(-c3ccc(-c4ccc5ccc6c7ccccc7ccc6c5c4)cc3)nc3ccccc32)cc1]

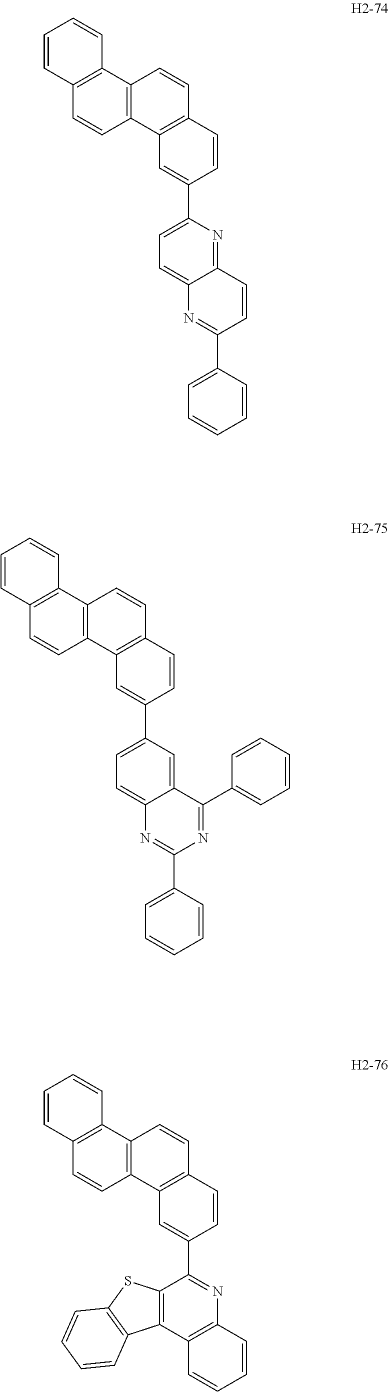 [SMILES: c1ccc(-c2ccc3nc(-c4ccc5ccc6c7ccccc7ccc6c5c4)ccc3n2)cc1 ; c1ccc(-c2nc(-c3ccccc3)c3cc(-c4ccc5ccc6c7ccccc7ccc6c5c4)ccc3n2)cc1 ; c1ccc2c(c1)ccc1c3cc(-c4nc5ccccc5c5c4sc4ccccc45)ccc3ccc21]

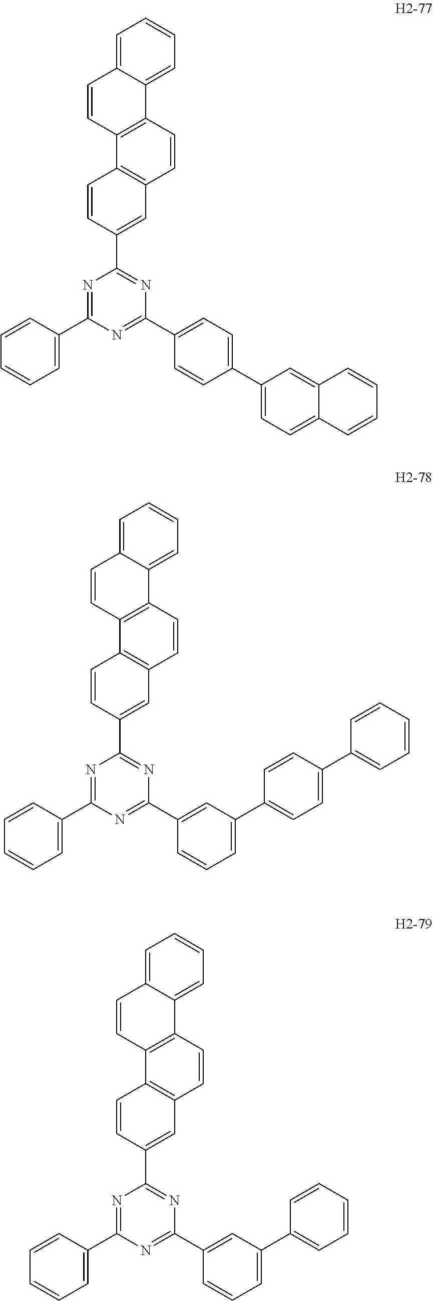 [SMILES: c1ccc(-c2nc(-c3ccc(-c4ccc5ccccc5c4)cc3)nc(-c3ccc4c(ccc5c6ccccc6ccc45)c3)n2)cc1 ; c1ccc(-c2ccc(-c3cccc(-c4nc(-c5ccccc5)nc(-c5ccc6c(ccc7c8ccccc8ccc67)c5)n4)c3)cc2)cc1 ; c1ccc(-c2cccc(-c3nc(-c4ccccc4)nc(-c4ccc5c(ccc6c7ccccc7ccc56)c4)n3)c2)cc1]

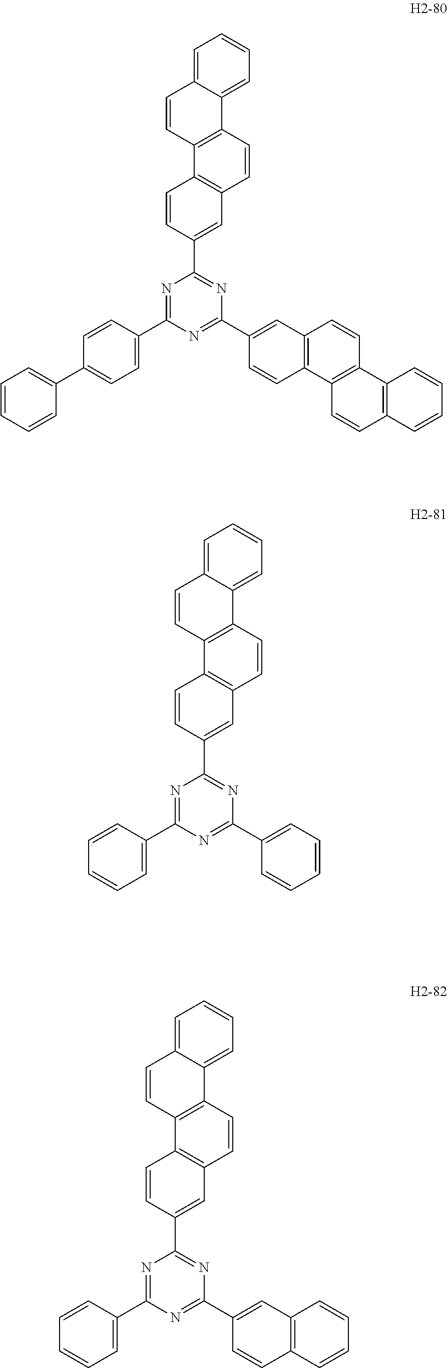 [SMILES: c1ccc(-c2ccc(-c3nc(-c4ccc5c(ccc6c7ccccc7ccc56)c4)nc(-c4ccc5c(ccc6c7ccccc7ccc56)c4)n3)cc2)cc1 ; c1ccc(-c2nc(-c3ccccc3)nc(-c3ccc4c(ccc5c6ccccc6ccc45)c3)n2)cc1 ; c1ccc(-c2nc(-c3ccc4ccccc4c3)nc(-c3ccc4c(ccc5c6ccccc6ccc45)c3)n2)cc1]

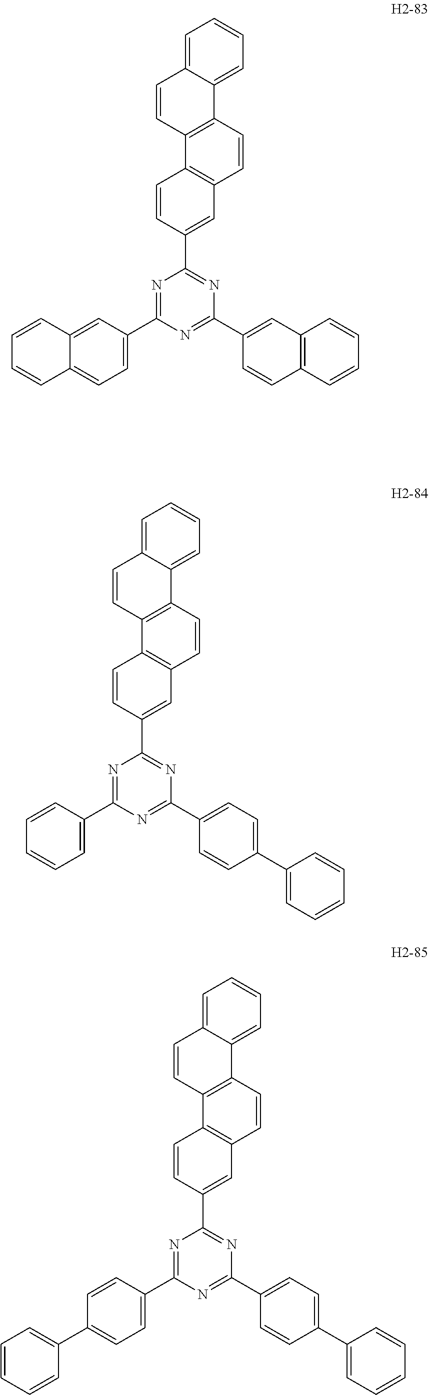 [SMILES: c1ccc2cc(-c3nc(-c4ccc5ccccc5c4)nc(-c4ccc5c(ccc6c7ccccc7ccc56)c4)n3)ccc2c1 ; c1ccc(-c2ccc(-c3nc(-c4ccccc4)nc(-c4ccc5c(ccc6c7ccccc7ccc56)c4)n3)cc2)cc1 ; c1ccc(-c2ccc(-c3nc(-c4ccc(-c5ccccc5)cc4)nc(-c4ccc5c(ccc6c7ccccc7ccc56)c4)n3)cc2)cc1]

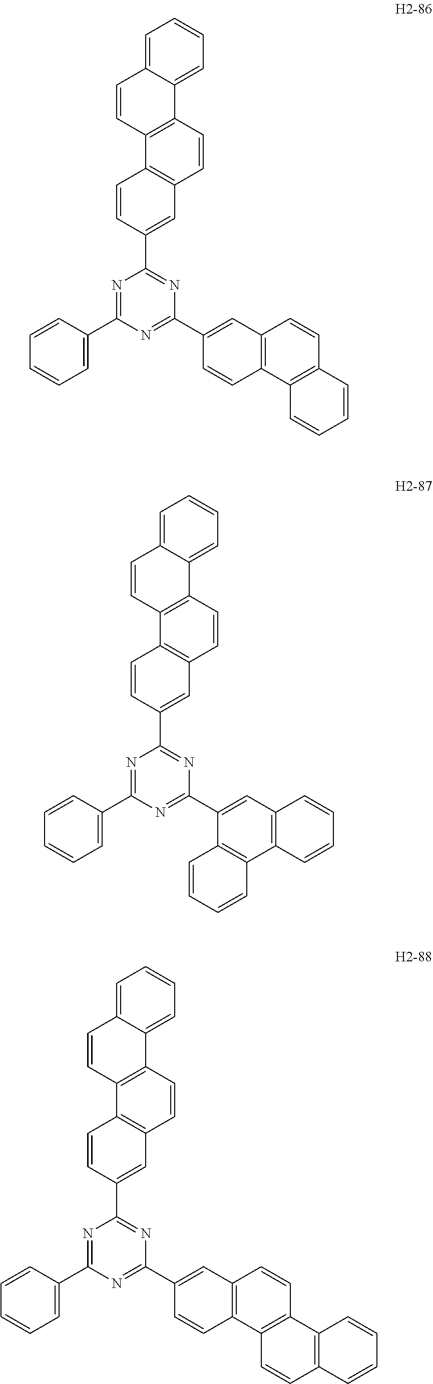 [SMILES: c1ccc(-c2nc(-c3ccc4c(ccc5ccccc54)c3)nc(-c3ccc4c(ccc5c6ccccc6ccc45)c3)n2)cc1 ; c1ccc(-c2nc(-c3ccc4c(ccc5c6ccccc6ccc45)c3)nc(-c3cc4ccccc4c4ccccc34)n2)cc1 ; c1ccc(-c2nc(-c3ccc4c(ccc5c6ccccc6ccc45)c3)nc(-c3ccc4c(ccc5c6ccccc6ccc45)c3)n2)cc1]

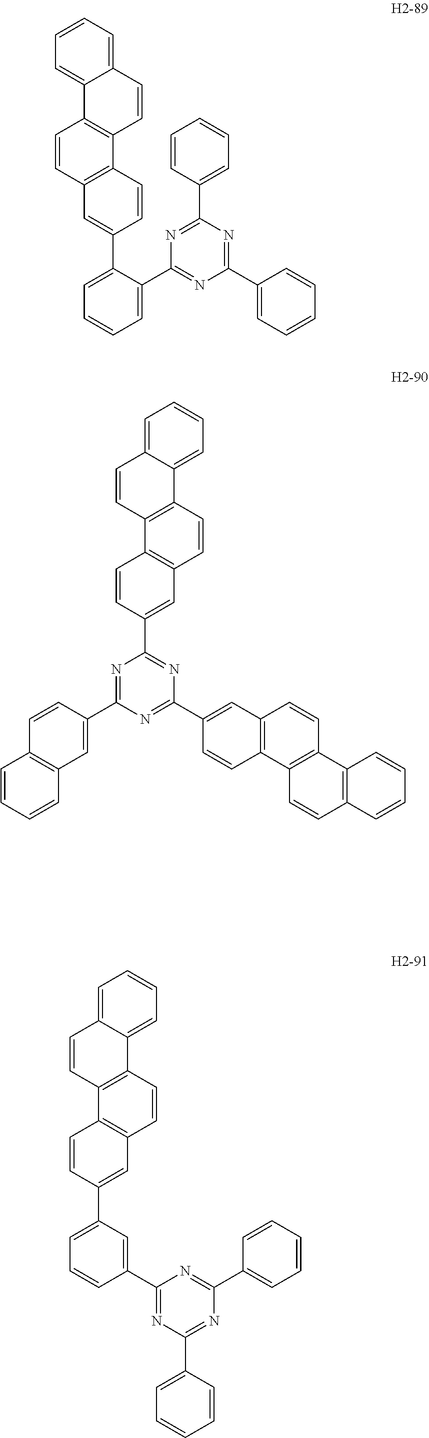 [SMILES: c1ccc(-c2nc(-c3ccccc3)nc(-c3ccccc3-c3ccc4c(ccc5c6ccccc6ccc45)c3)n2)cc1 ; c1ccc2cc(-c3nc(-c4ccc5c(ccc6c7ccccc7ccc56)c4)nc(-c4ccc5c(ccc6c7ccccc7ccc56)c4)n3)ccc2c1 ; c1ccc(-c2nc(-c3ccccc3)nc(-c3cccc(-c4ccc5c(ccc6c7ccccc7ccc56)c4)c3)n2)cc1]

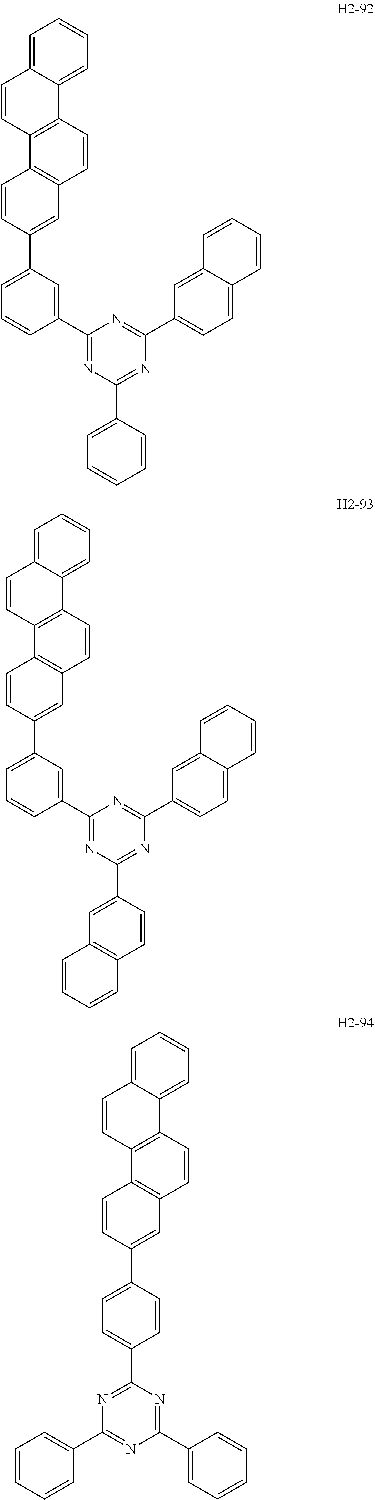 [SMILES: c1ccc(-c2nc(-c3cccc(-c4ccc5c(ccc6c7ccccc7ccc56)c4)c3)nc(-c3ccc4ccccc4c3)n2)cc1 ; c1cc(-c2ccc3c(ccc4c5ccccc5ccc34)c2)cc(-c2nc(-c3ccc4ccccc4c3)nc(-c3ccc4ccccc4c3)n2)c1 ; c1ccc(-c2nc(-c3ccccc3)nc(-c3ccc(-c4ccc5c(ccc6c7ccccc7ccc56)c4)cc3)n2)cc1]

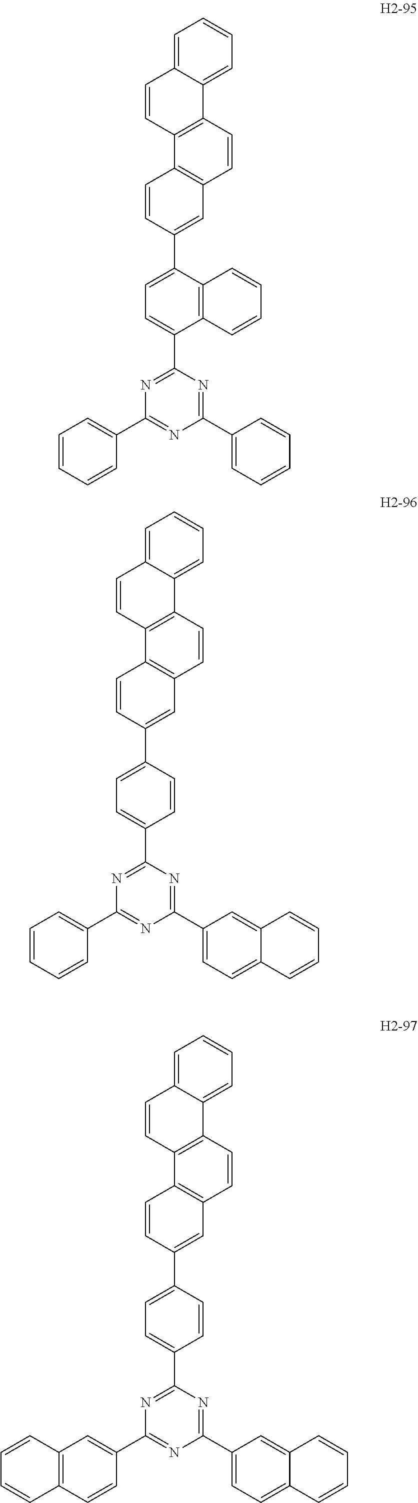 [SMILES: c1ccc(-c2nc(-c3ccccc3)nc(-c3ccc(-c4ccc5c(ccc6c7ccccc7ccc56)c4)c4ccccc34)n2)cc1 ; c1ccc(-c2nc(-c3ccc(-c4ccc5c(ccc6c7ccccc7ccc56)c4)cc3)nc(-c3ccc4ccccc4c3)n2)cc1 ; c1ccc2cc(-c3nc(-c4ccc(-c5ccc6c(ccc7c8ccccc8ccc67)c5)cc4)nc(-c4ccc5ccccc5c4)n3)ccc2c1]

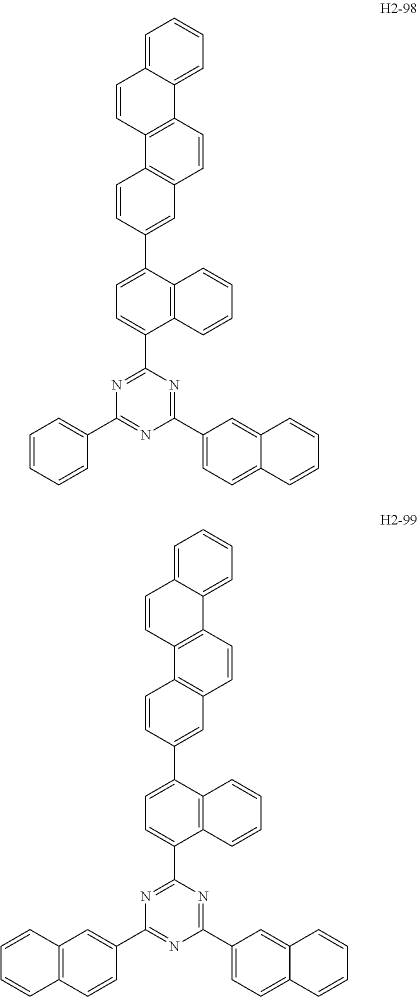 [SMILES: c1ccc(-c2nc(-c3ccc4ccccc4c3)nc(-c3ccc(-c4ccc5c(ccc6c7ccccc7ccc56)c4)c4ccccc34)n2)cc1 ; c1ccc2cc(-c3nc(-c4ccc5ccccc5c4)nc(-c4ccc(-c5ccc6c(ccc7c8ccccc8ccc67)c5)c5ccccc45)n3)ccc2c1]

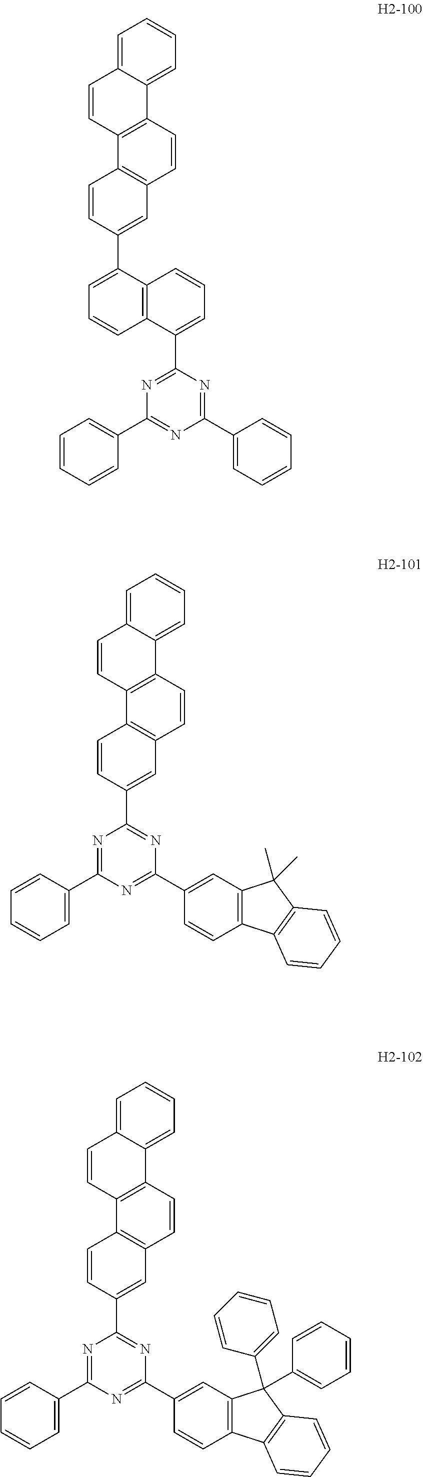 [SMILES: c1ccc(-c2nc(-c3ccccc3)nc(-c3cccc4c(-c5ccc6c(ccc7c8ccccc8ccc67)c5)cccc34)n2)cc1 ; CC1(C)c2ccccc2-c2ccc(-c3nc(-c4ccccc4)nc(-c4ccc5c(ccc6c7ccccc7ccc56)c4)n3)cc21 ; c1ccc(-c2nc(-c3ccc4c(c3)C(c3ccccc3)(c3ccccc3)c3ccccc3-4)nc(-c3ccc4c(ccc5c6ccccc6ccc45)c3)n2)cc1]

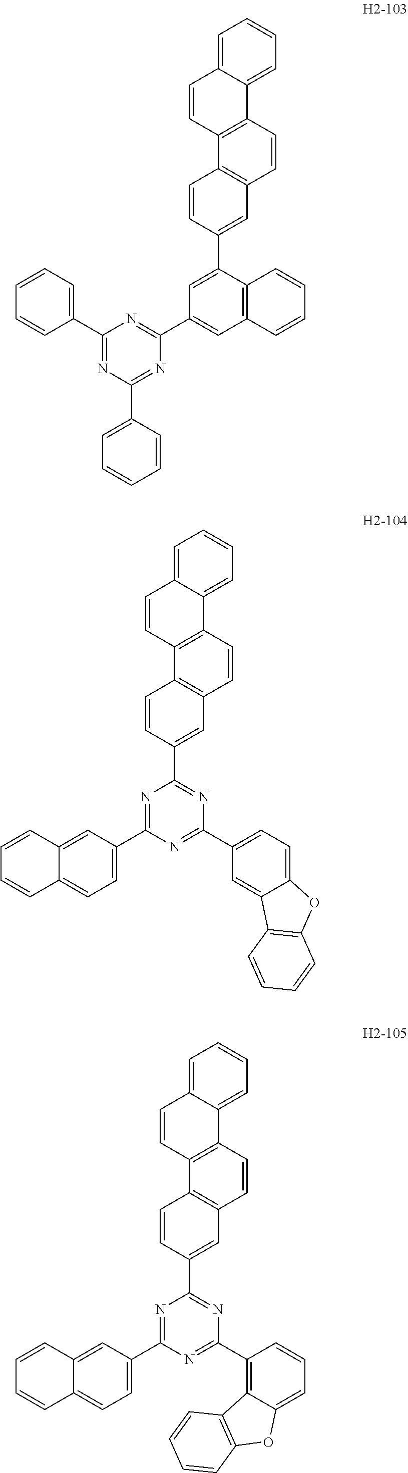 [SMILES: c1ccc(-c2nc(-c3ccccc3)nc(-c3cc(-c4ccc5c(ccc6c7ccccc7ccc56)c4)c4ccccc4c3)n2)cc1 ; c1ccc2cc(-c3nc(-c4ccc5c(ccc6c7ccccc7ccc56)c4)nc(-c4ccc5oc6ccccc6c5c4)n3)ccc2c1 ; c1ccc2cc(-c3nc(-c4ccc5c(ccc6c7ccccc7ccc56)c4)nc(-c4cccc5oc6ccccc6c45)n3)ccc2c1]

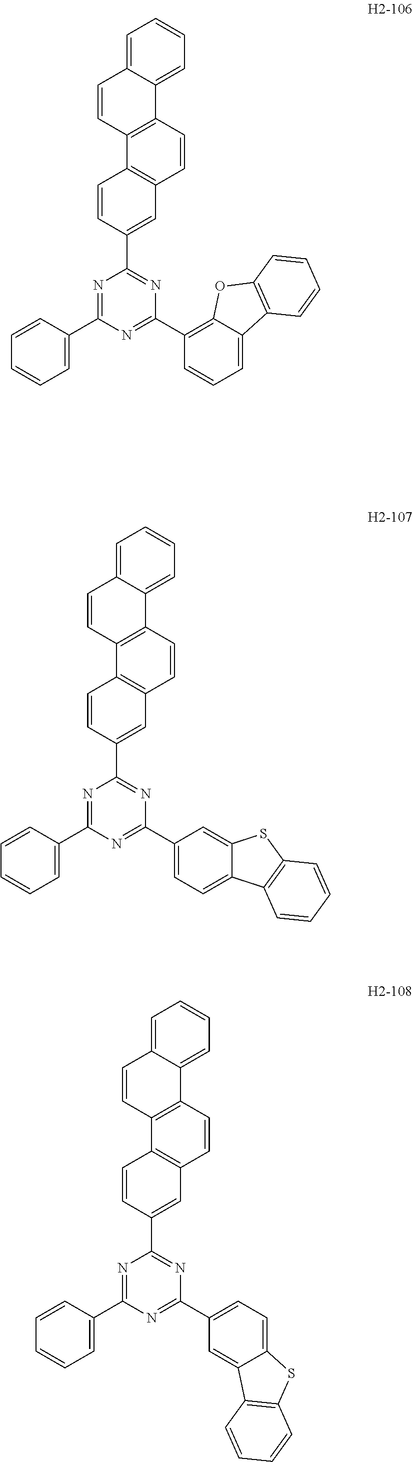 [SMILES: c1ccc(-c2nc(-c3ccc4c(ccc5c6ccccc6ccc45)c3)nc(-c3cccc4c3oc3ccccc34)n2)cc1 ; c1ccc(-c2nc(-c3ccc4c(ccc5c6ccccc6ccc45)c3)nc(-c3ccc4c(c3)sc3ccccc34)n2)cc1 ; c1ccc(-c2nc(-c3ccc4c(ccc5c6ccccc6ccc45)c3)nc(-c3ccc4sc5ccccc5c4c3)n2)cc1]

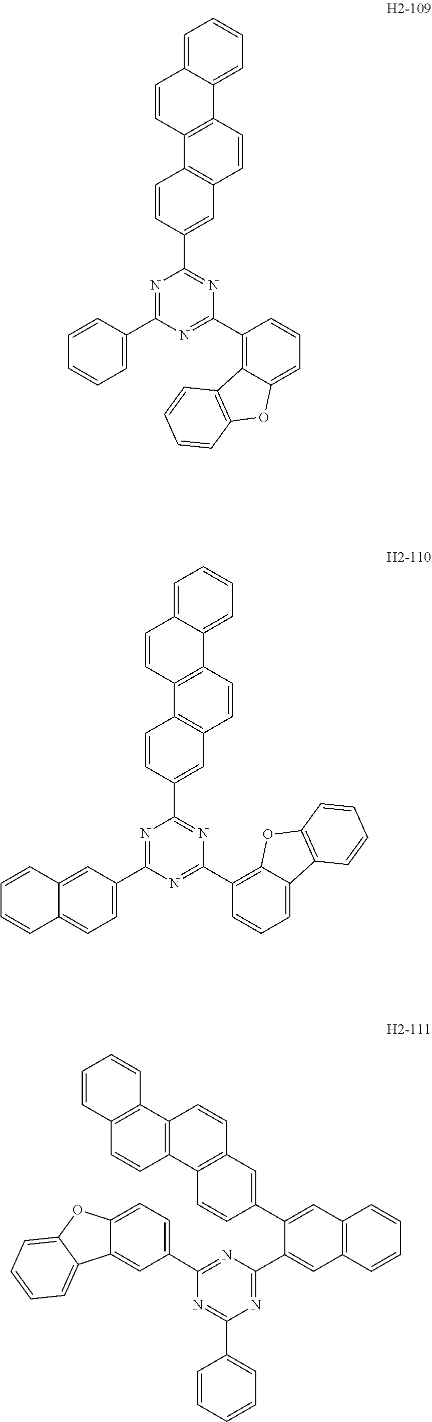 [SMILES: c1ccc(-c2nc(-c3ccc4c(ccc5c6ccccc6ccc45)c3)nc(-c3cccc4oc5ccccc5c34)n2)cc1 ; c1ccc2cc(-c3nc(-c4ccc5c(ccc6c7ccccc7ccc56)c4)nc(-c4cccc5c4oc4ccccc45)n3)ccc2c1 ; c1ccc(-c2nc(-c3ccc4oc5ccccc5c4c3)nc(-c3cc4ccccc4cc3-c3ccc4c(ccc5c6ccccc6ccc45)c3)n2)cc1]

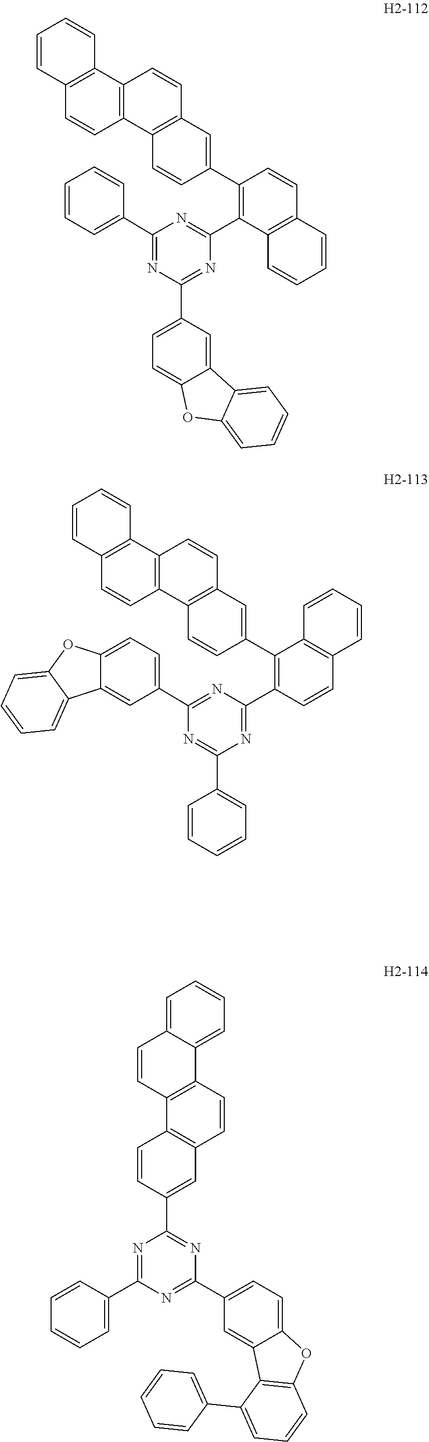 [SMILES: c1ccc(-c2nc(-c3ccc4oc5ccccc5c4c3)nc(-c3c(-c4ccc5c(ccc6c7ccccc7ccc56)c4)ccc4ccccc34)n2)cc1 ; c1ccc(-c2nc(-c3ccc4oc5ccccc5c4c3)nc(-c3ccc4ccccc4c3-c3ccc4c(ccc5c6ccccc6ccc45)c3)n2)cc1 ; c1ccc(-c2nc(-c3ccc4c(ccc5c6ccccc6ccc45)c3)nc(-c3ccc4oc5cccc(-c6ccccc6)c5c4c3)n2)cc1]

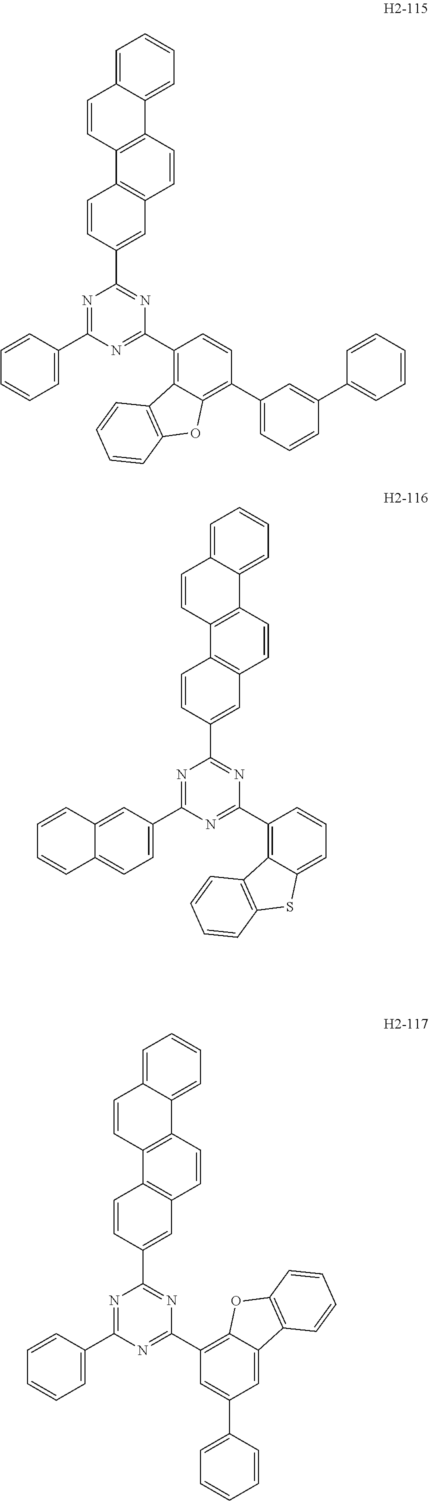 [SMILES: c1ccc(-c2cccc(-c3ccc(-c4nc(-c5ccccc5)nc(-c5ccc6c(ccc7c8ccccc8ccc67)c5)n4)c4c3oc3ccccc34)c2)cc1 ; c1ccc2cc(-c3nc(-c4ccc5c(ccc6c7ccccc7ccc56)c4)nc(-c4cccc5sc6ccccc6c45)n3)ccc2c1 ; c1ccc(-c2cc(-c3nc(-c4ccccc4)nc(-c4ccc5c(ccc6c7ccccc7ccc56)c4)n3)c3oc4ccccc4c3c2)cc1]

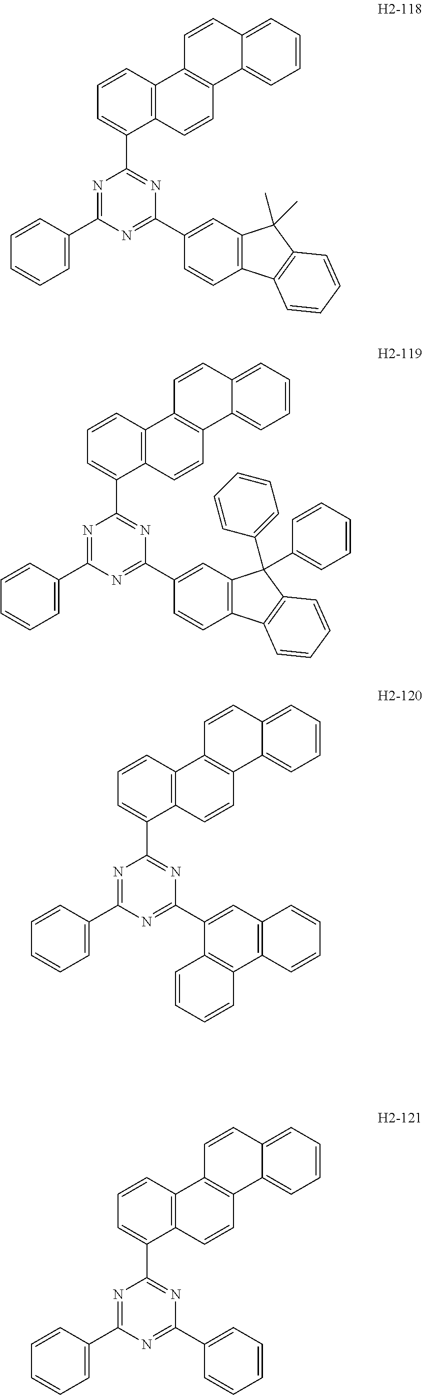 [SMILES: CC1(C)c2ccccc2-c2ccc(-c3nc(-c4ccccc4)nc(-c4cccc5c4ccc4c6ccccc6ccc54)n3)cc21 ; c1ccc(-c2nc(-c3ccc4c(c3)C(c3ccccc3)(c3ccccc3)c3ccccc3-4)nc(-c3cccc4c3ccc3c5ccccc5ccc43)n2)cc1 ; c1ccc(-c2nc(-c3cc4ccccc4c4ccccc34)nc(-c3cccc4c3ccc3c5ccccc5ccc43)n2)cc1 ; c1ccc(-c2nc(-c3ccccc3)nc(-c3cccc4c3ccc3c5ccccc5ccc43)n2)cc1]

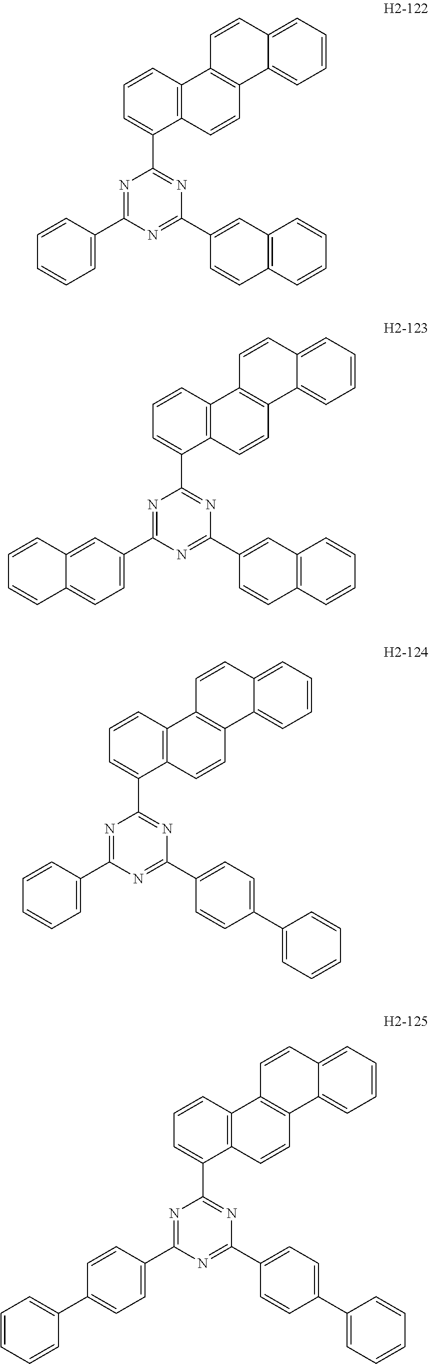 [SMILES: c1ccc(-c2nc(-c3ccc4ccccc4c3)nc(-c3cccc4c3ccc3c5ccccc5ccc43)n2)cc1 ; c1ccc2cc(-c3nc(-c4ccc5ccccc5c4)nc(-c4cccc5c4ccc4c6ccccc6ccc54)n3)ccc2c1 ; c1ccc(-c2ccc(-c3nc(-c4ccccc4)nc(-c4cccc5c4ccc4c6ccccc6ccc54)n3)cc2)cc1 ; c1ccc(-c2ccc(-c3nc(-c4ccc(-c5ccccc5)cc4)nc(-c4cccc5c4ccc4c6ccccc6ccc54)n3)cc2)cc1]

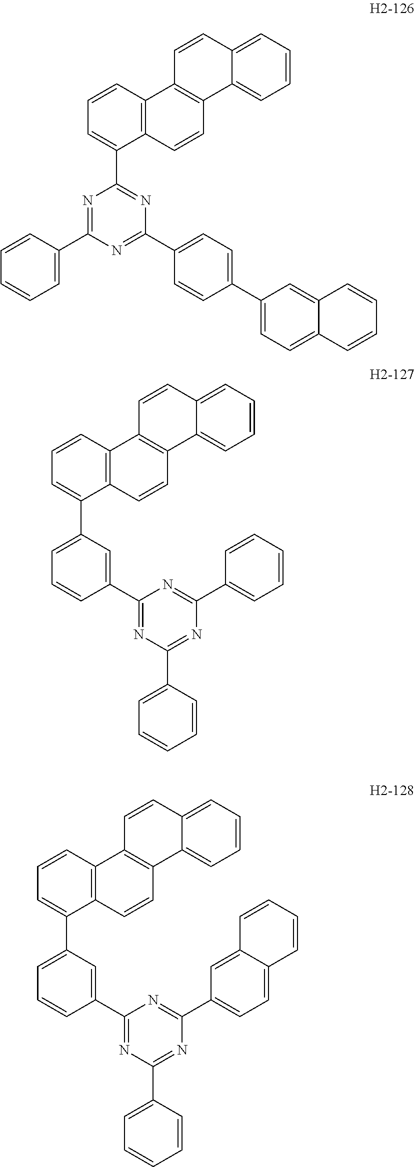 [SMILES: c1ccc(-c2nc(-c3ccc(-c4ccc5ccccc5c4)cc3)nc(-c3cccc4c3ccc3c5ccccc5ccc43)n2)cc1 ; c1ccc(-c2nc(-c3ccccc3)nc(-c3cccc(-c4cccc5c4ccc4c6ccccc6ccc54)c3)n2)cc1 ; c1ccc(-c2nc(-c3cccc(-c4cccc5c4ccc4c6ccccc6ccc54)c3)nc(-c3ccc4ccccc4c3)n2)cc1]

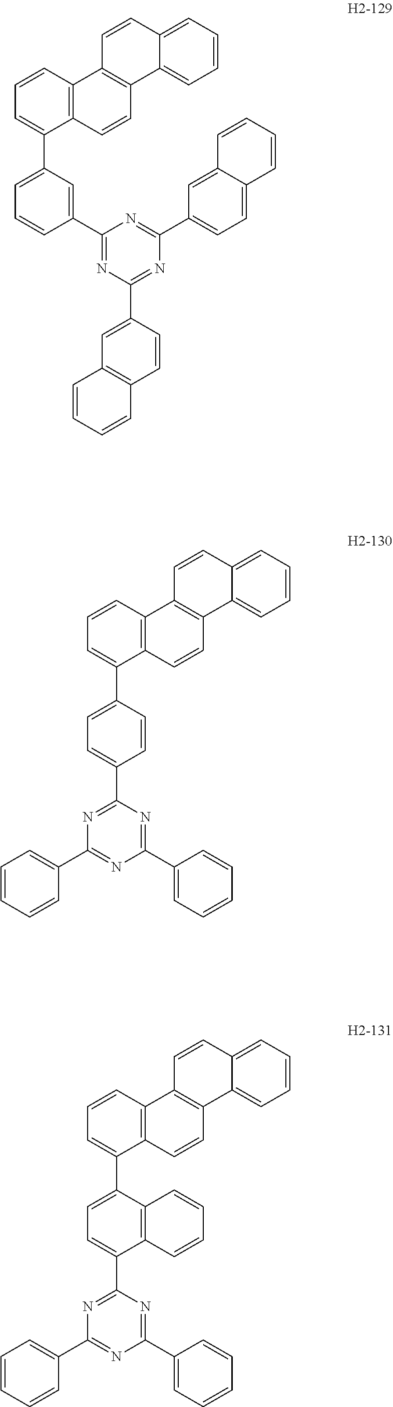 [SMILES: c1cc(-c2nc(-c3ccc4ccccc4c3)nc(-c3ccc4ccccc4c3)n2)cc(-c2cccc3c2ccc2c4ccccc4ccc32)c1 ; c1ccc(-c2nc(-c3ccccc3)nc(-c3ccc(-c4cccc5c4ccc4c6ccccc6ccc54)cc3)n2)cc1 ; c1ccc(-c2nc(-c3ccccc3)nc(-c3ccc(-c4cccc5c4ccc4c6ccccc6ccc54)c4ccccc34)n2)cc1]

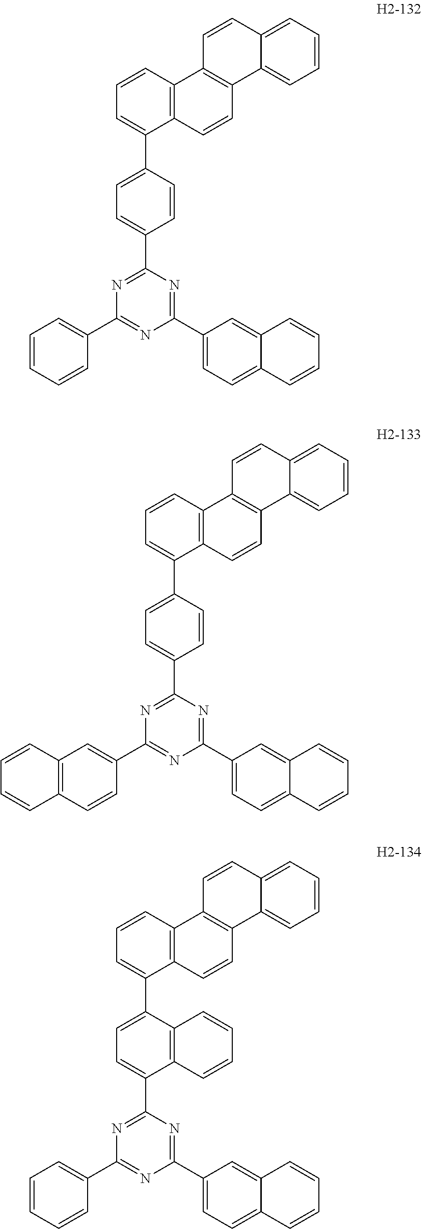 [SMILES: c1ccc(-c2nc(-c3ccc(-c4cccc5c4ccc4c6ccccc6ccc54)cc3)nc(-c3ccc4ccccc4c3)n2)cc1 ; c1ccc2cc(-c3nc(-c4ccc(-c5cccc6c5ccc5c7ccccc7ccc65)cc4)nc(-c4ccc5ccccc5c4)n3)ccc2c1 ; c1ccc(-c2nc(-c3ccc4ccccc4c3)nc(-c3ccc(-c4cccc5c4ccc4c6ccccc6ccc54)c4ccccc34)n2)cc1]

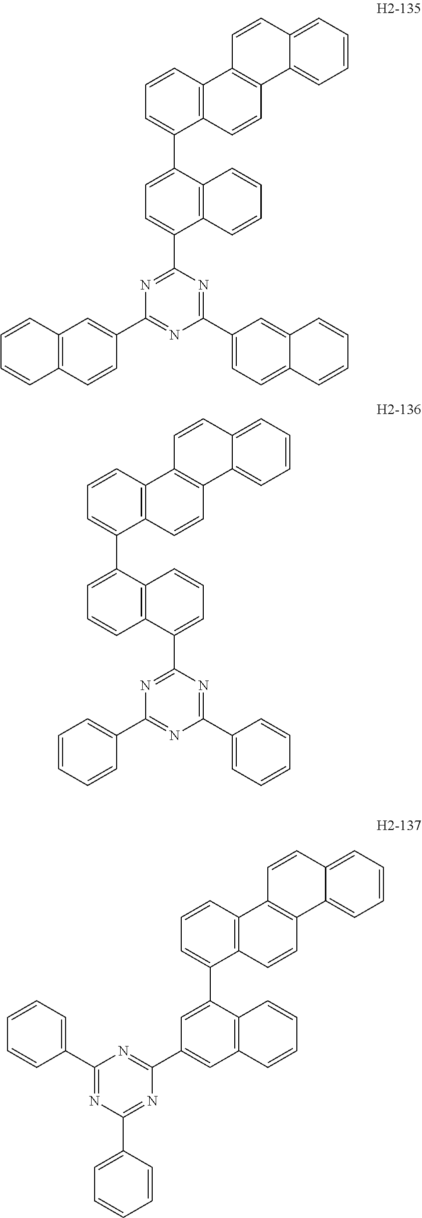

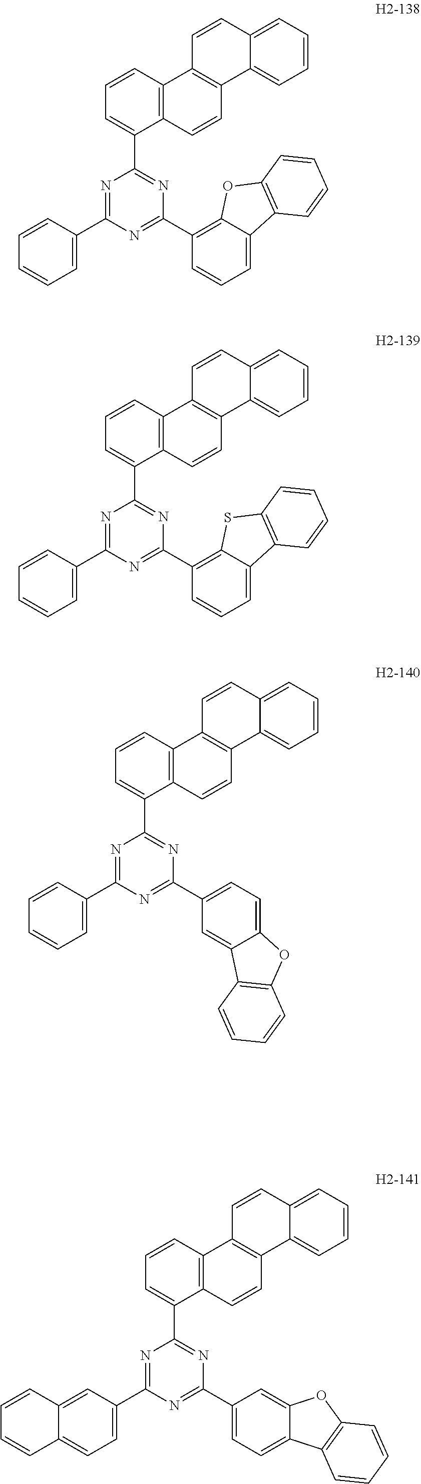 [SMILES: c1ccc(-c2nc(-c3cccc4c3ccc3c5ccccc5ccc43)nc(-c3cccc4c3oc3ccccc34)n2)cc1 ; c1ccc(-c2nc(-c3cccc4c3ccc3c5ccccc5ccc43)nc(-c3cccc4c3sc3ccccc34)n2)cc1 ; c1ccc(-c2nc(-c3ccc4oc5ccccc5c4c3)nc(-c3cccc4c3ccc3c5ccccc5ccc43)n2)cc1 ; c1ccc2cc(-c3nc(-c4ccc5c(c4)oc4ccccc45)nc(-c4cccc5c4ccc4c6ccccc6ccc54)n3)ccc2c1]

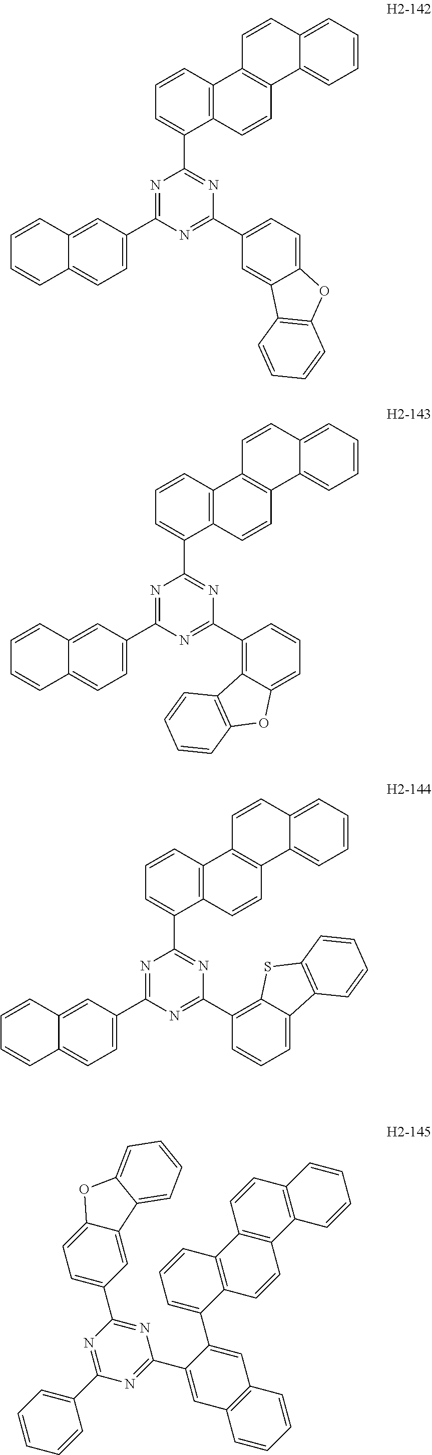 [SMILES: c1ccc2cc(-c3nc(-c4ccc5oc6ccccc6c5c4)nc(-c4cccc5c4ccc4c6ccccc6ccc54)n3)ccc2c1 ; c1ccc2cc(-c3nc(-c4cccc5c4ccc4c6ccccc6ccc54)nc(-c4cccc5oc6ccccc6c45)n3)ccc2c1 ; c1ccc2cc(-c3nc(-c4cccc5c4ccc4c6ccccc6ccc54)nc(-c4cccc5c4sc4ccccc45)n3)ccc2c1 ; c1ccc(-c2nc(-c3ccc4oc5ccccc5c4c3)nc(-c3cc4ccccc4cc3-c3cccc4c3ccc3c5ccccc5ccc43)n2)cc1]

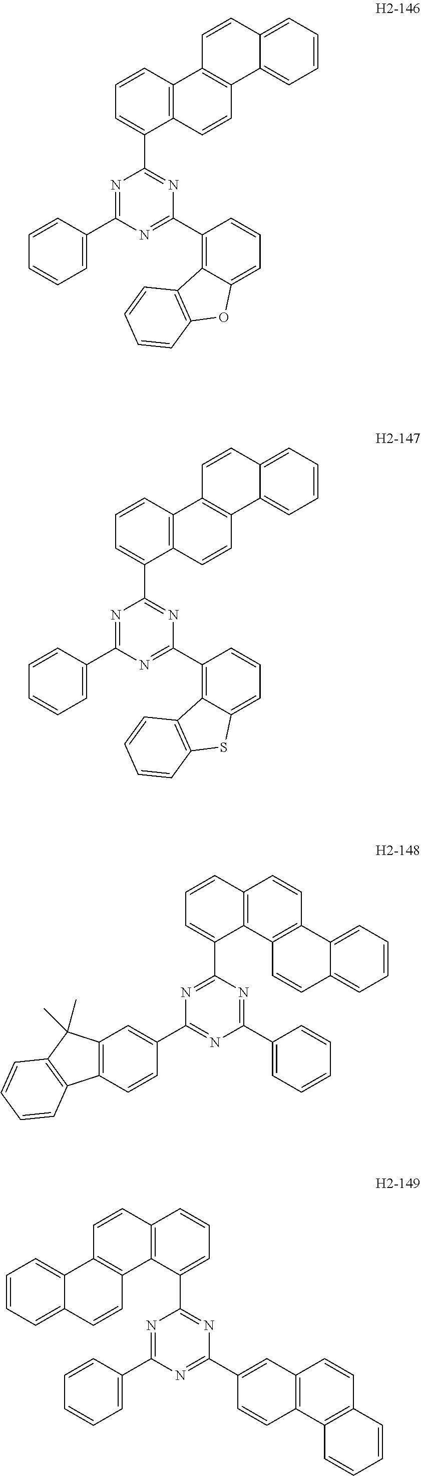 [SMILES: c1ccc(-c2nc(-c3cccc4c3ccc3c5ccccc5ccc43)nc(-c3cccc4oc5ccccc5c34)n2)cc1 ; c1ccc(-c2nc(-c3cccc4c3ccc3c5ccccc5ccc43)nc(-c3cccc4sc5ccccc5c34)n2)cc1 ; CC1(C)c2ccccc2-c2ccc(-c3nc(-c4ccccc4)nc(-c4cccc5ccc6c7ccccc7ccc6c45)n3)cc21 ; c1ccc(-c2nc(-c3ccc4c(ccc5ccccc54)c3)nc(-c3cccc4ccc5c6ccccc6ccc5c34)n2)cc1]

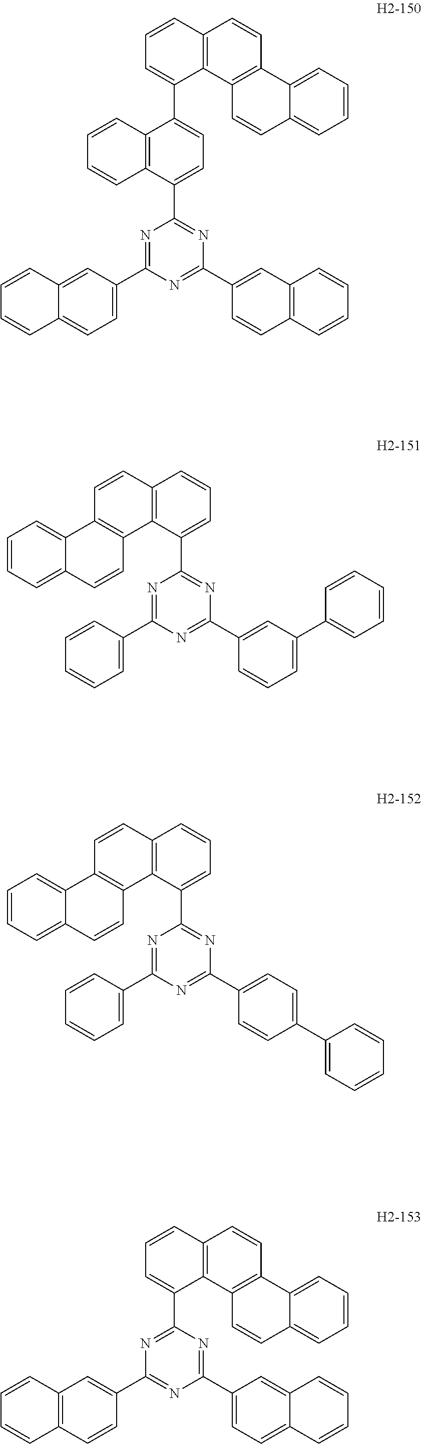 [SMILES: c1ccc2cc(-c3nc(-c4ccc5ccccc5c4)nc(-c4ccc(-c5cccc6ccc7c8ccccc8ccc7c56)c5ccccc45)n3)ccc2c1 ; c1ccc(-c2cccc(-c3nc(-c4ccccc4)nc(-c4cccc5ccc6c7ccccc7ccc6c45)n3)c2)cc1 ; c1ccc(-c2ccc(-c3nc(-c4ccccc4)nc(-c4cccc5ccc6c7ccccc7ccc6c45)n3)cc2)cc1 ; c1ccc2cc(-c3nc(-c4ccc5ccccc5c4)nc(-c4cccc5ccc6c7ccccc7ccc6c45)n3)ccc2c1]

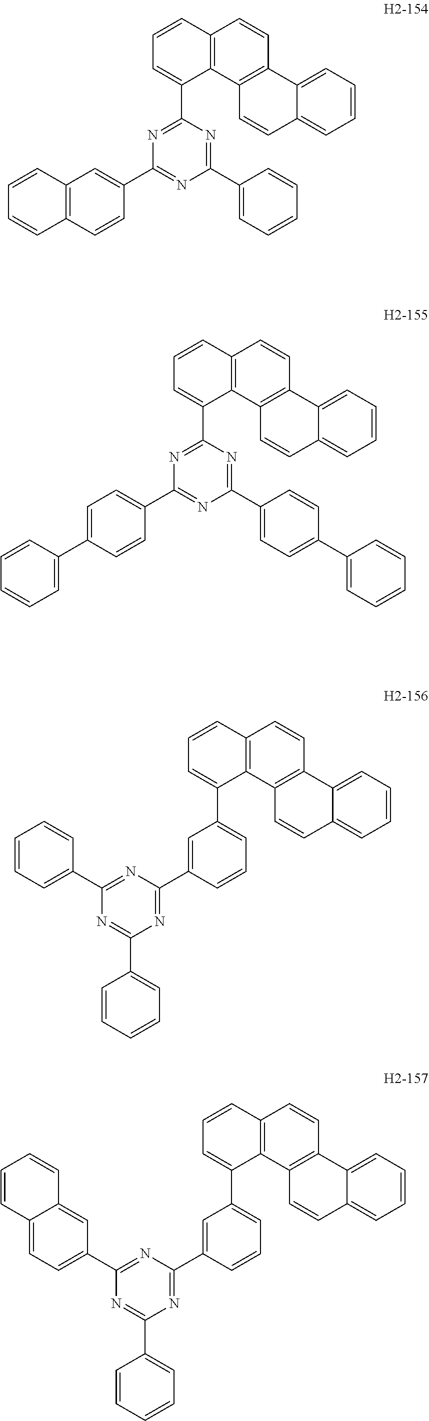 [SMILES: c1ccc(-c2nc(-c3ccc4ccccc4c3)nc(-c3cccc4ccc5c6ccccc6ccc5c34)n2)cc1 ; c1ccc(-c2ccc(-c3nc(-c4ccc(-c5ccccc5)cc4)nc(-c4cccc5ccc6c7ccccc7ccc6c45)n3)cc2)cc1 ; c1ccc(-c2nc(-c3ccccc3)nc(-c3cccc(-c4cccc5ccc6c7ccccc7ccc6c45)c3)n2)cc1 ; c1ccc(-c2nc(-c3cccc(-c4cccc5ccc6c7ccccc7ccc6c45)c3)nc(-c3ccc4ccccc4c3)n2)cc1]

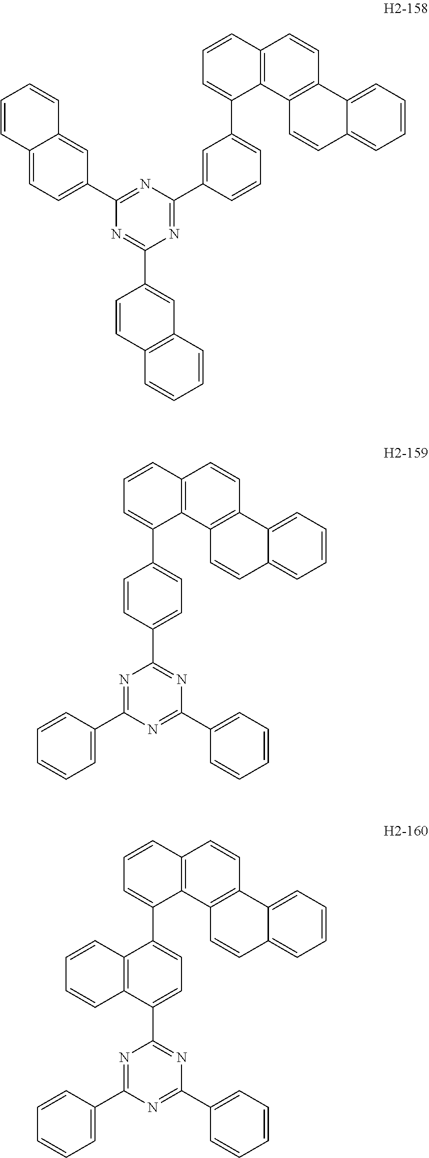 [SMILES: c1cc(-c2nc(-c3ccc4ccccc4c3)nc(-c3ccc4ccccc4c3)n2)cc(-c2cccc3ccc4c5ccccc5ccc4c23)c1 ; c1ccc(-c2nc(-c3ccccc3)nc(-c3ccc(-c4cccc5ccc6c7ccccc7ccc6c45)cc3)n2)cc1 ; c1ccc(-c2nc(-c3ccccc3)nc(-c3ccc(-c4cccc5ccc6c7ccccc7ccc6c45)c4ccccc34)n2)cc1]

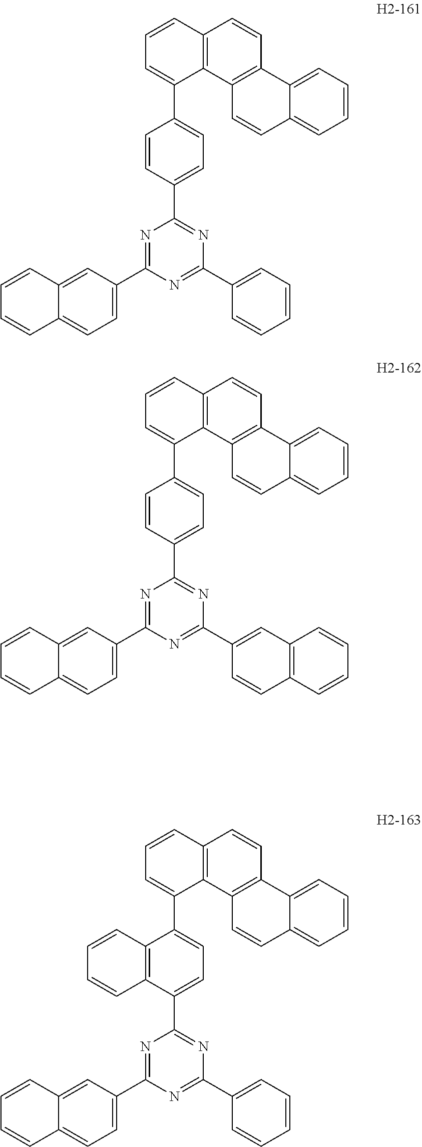 [SMILES: c1ccc(-c2nc(-c3ccc(-c4cccc5ccc6c7ccccc7ccc6c45)cc3)nc(-c3ccc4ccccc4c3)n2)cc1 ; c1ccc2cc(-c3nc(-c4ccc(-c5cccc6ccc7c8ccccc8ccc7c56)cc4)nc(-c4ccc5ccccc5c4)n3)ccc2c1 ; c1ccc(-c2nc(-c3ccc4ccccc4c3)nc(-c3ccc(-c4cccc5ccc6c7ccccc7ccc6c45)c4ccccc34)n2)cc1]

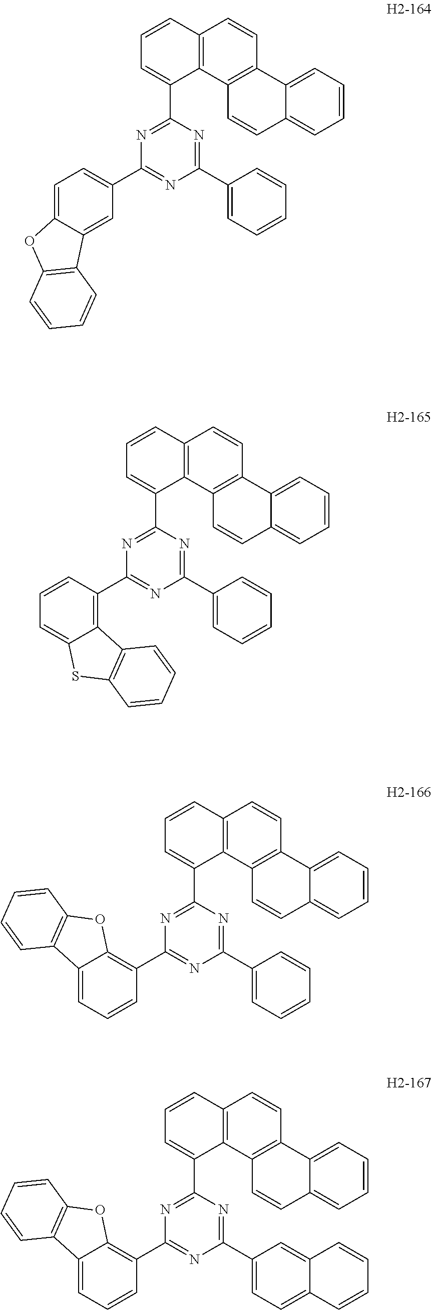 [SMILES: c1ccc(-c2nc(-c3ccc4oc5ccccc5c4c3)nc(-c3cccc4ccc5c6ccccc6ccc5c34)n2)cc1 ; c1ccc(-c2nc(-c3cccc4ccc5c6ccccc6ccc5c34)nc(-c3cccc4sc5ccccc5c34)n2)cc1 ; c1ccc(-c2nc(-c3cccc4c3oc3ccccc34)nc(-c3cccc4ccc5c6ccccc6ccc5c34)n2)cc1 ; c1ccc2cc(-c3nc(-c4cccc5c4oc4ccccc45)nc(-c4cccc5ccc6c7ccccc7ccc6c45)n3)ccc2c1]

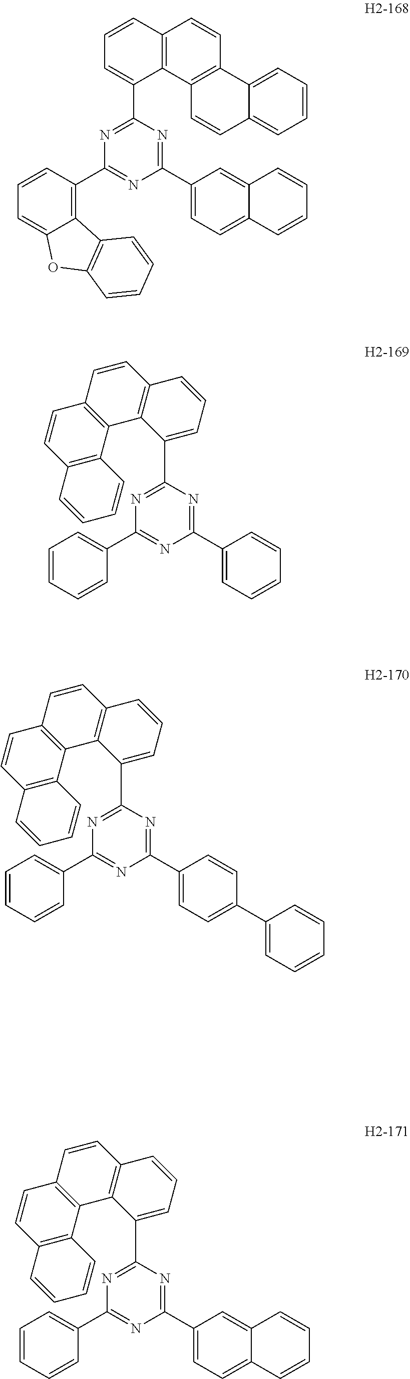 [SMILES: c1ccc2cc(-c3nc(-c4cccc5ccc6c7ccccc7ccc6c45)nc(-c4cccc5oc6ccccc6c45)n3)ccc2c1 ; c1ccc(-c2nc(-c3ccccc3)nc(-c3cccc4ccc5ccc6ccccc6c5c34)n2)cc1 ; c1ccc(-c2ccc(-c3nc(-c4ccccc4)nc(-c4cccc5ccc6ccc7ccccc7c6c45)n3)cc2)cc1 ; c1ccc(-c2nc(-c3ccc4ccccc4c3)nc(-c3cccc4ccc5ccc6ccccc6c5c34)n2)cc1]

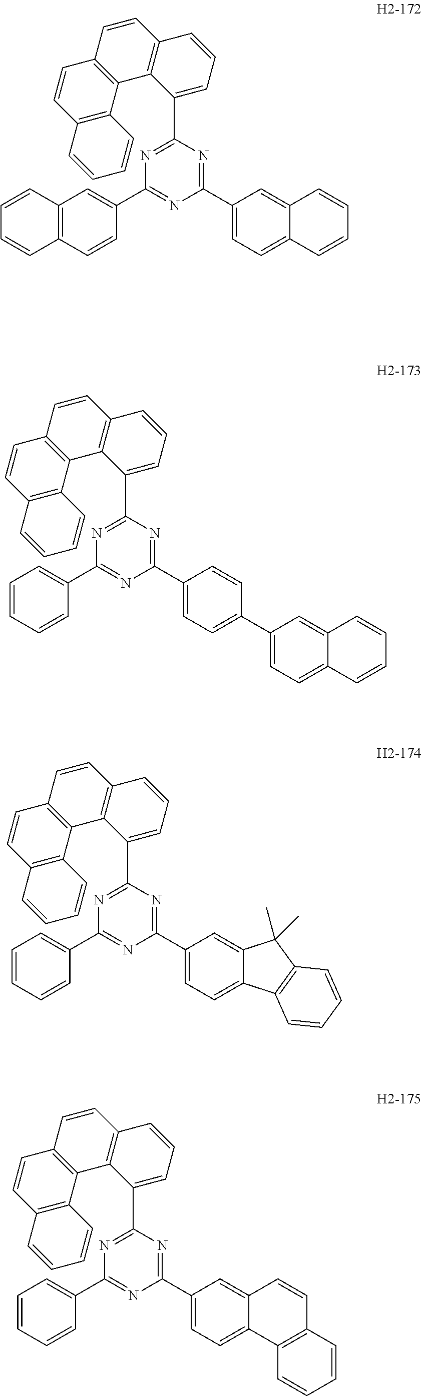 [SMILES: c1ccc2cc(-c3nc(-c4ccc5ccccc5c4)nc(-c4cccc5ccc6ccc7ccccc7c6c45)n3)ccc2c1 ; c1ccc(-c2nc(-c3ccc(-c4ccc5ccccc5c4)cc3)nc(-c3cccc4ccc5ccc6ccccc6c5c34)n2)cc1 ; CC1(C)c2ccccc2-c2ccc(-c3nc(-c4ccccc4)nc(-c4cccc5ccc6ccc7ccccc7c6c45)n3)cc21 ; c1ccc(-c2nc(-c3ccc4c(ccc5ccccc54)c3)nc(-c3cccc4ccc5ccc6ccccc6c5c34)n2)cc1]

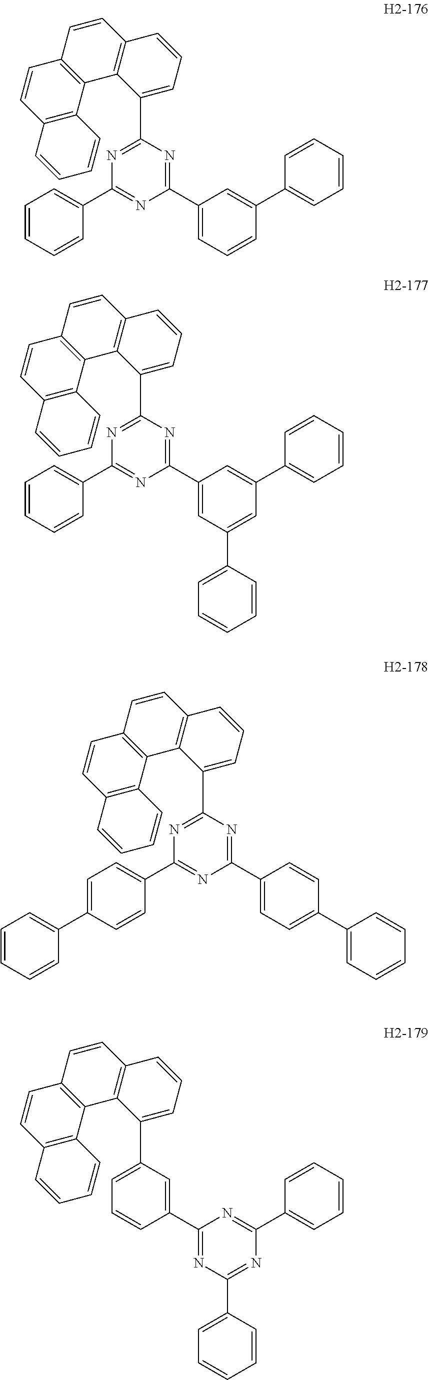

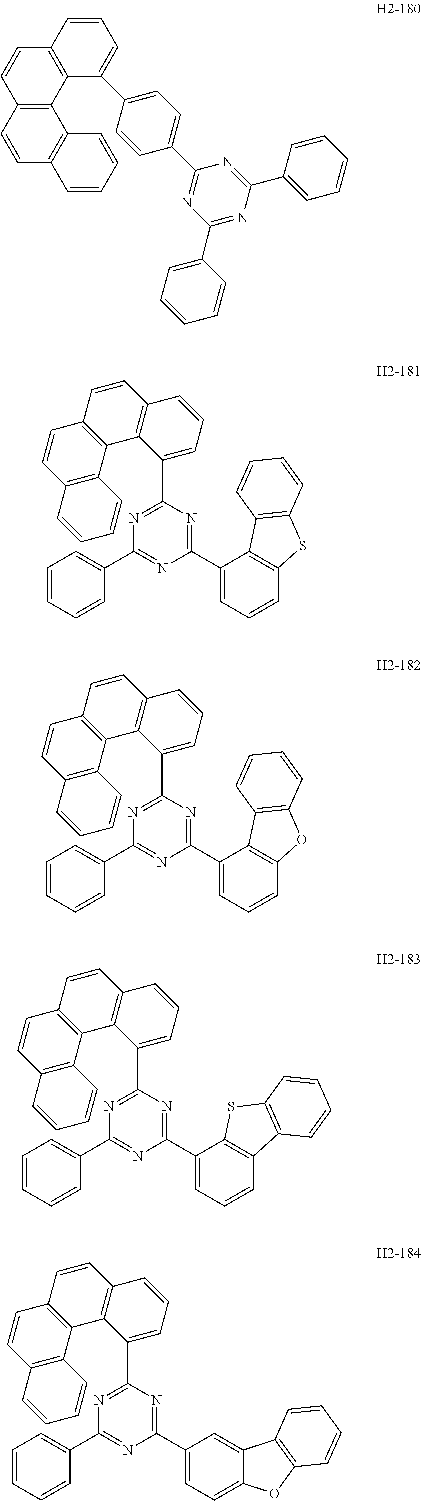 [SMILES: c1ccc(-c2nc(-c3ccccc3)nc(-c3ccc(-c4cccc5ccc6ccc7ccccc7c6c45)cc3)n2)cc1 ; c1ccc(-c2nc(-c3cccc4sc5ccccc5c34)nc(-c3cccc4ccc5ccc6ccccc6c5c34)n2)cc1 ; c1ccc(-c2nc(-c3cccc4oc5ccccc5c34)nc(-c3cccc4ccc5ccc6ccccc6c5c34)n2)cc1 ; c1ccc(-c2nc(-c3cccc4c3sc3ccccc34)nc(-c3cccc4ccc5ccc6ccccc6c5c34)n2)cc1 ; c1ccc(-c2nc(-c3ccc4oc5ccccc5c4c3)nc(-c3cccc4ccc5ccc6ccccc6c5c34)n2)cc1]

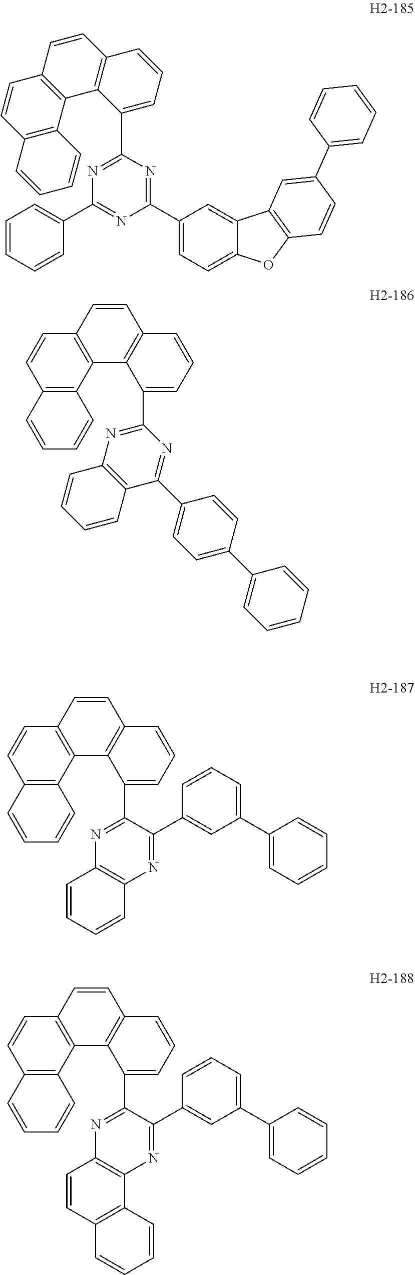 [SMILES: c1ccc(-c2ccc3oc4ccc(-c5nc(-c6ccccc6)nc(-c6cccc7ccc8ccc9ccccc9c8c67)n5)cc4c3c2)cc1 ; c1ccc(-c2ccc(-c3nc(-c4cccc5ccc6ccc7ccccc7c6c45)nc4ccccc34)cc2)cc1 ; c1ccc(-c2cccc(-c3nc4ccccc4nc3-c3cccc4ccc5ccc6ccccc6c5c34)c2)cc1 ; c1ccc(-c2cccc(-c3nc4c(ccc5ccccc54)nc3-c3cccc4ccc5ccc6ccccc6c5c34)c2)cc1]

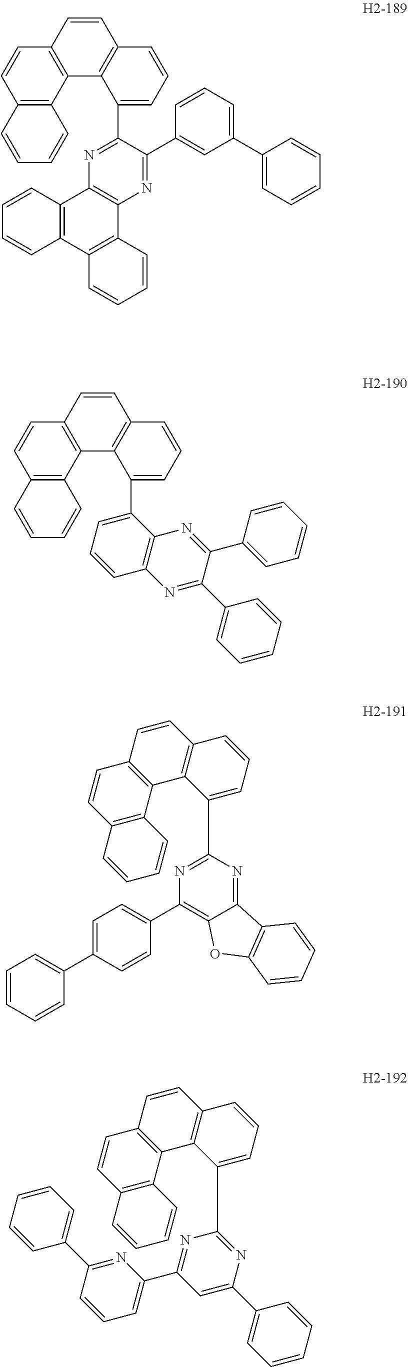 [SMILES: c1ccc(-c2cccc(-c3nc4c5ccccc5c5ccccc5c4nc3-c3cccc4ccc5ccc6ccccc6c5c34)c2)cc1 ; c1ccc(-c2nc3cccc(-c4cccc5ccc6ccc7ccccc7c6c45)c3nc2-c2ccccc2)cc1 ; c1ccc(-c2ccc(-c3nc(-c4cccc5ccc6ccc7ccccc7c6c45)nc4c3oc3ccccc34)cc2)cc1 ; c1ccc(-c2cccc(-c3cc(-c4ccccc4)nc(-c4cccc5ccc6ccc7ccccc7c6c45)n3)n2)cc1]

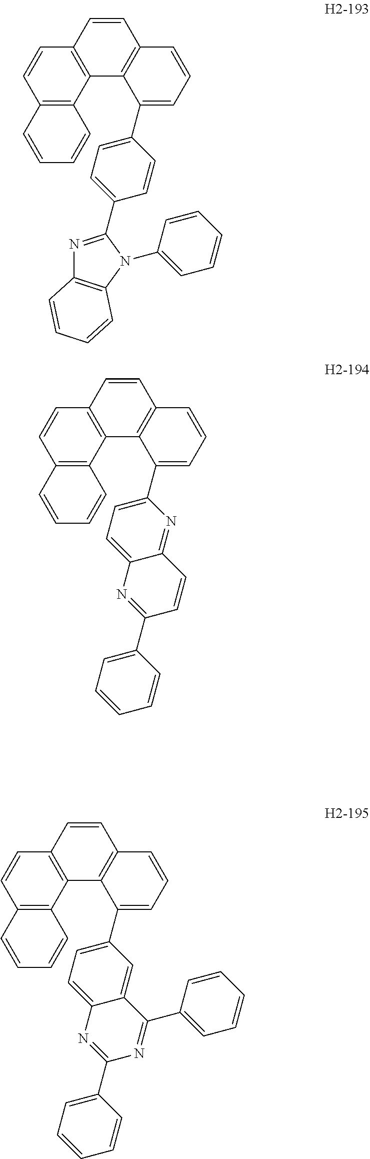

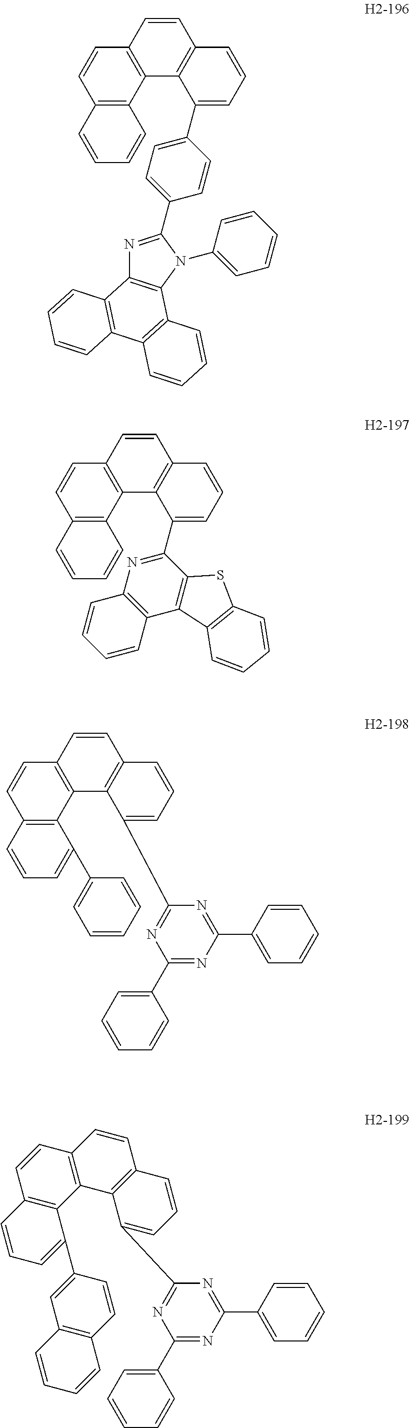 [SMILES: c1ccc(-n2c(-c3ccc(-c4cccc5ccc6ccc7ccccc7c6c45)cc3)nc3c4ccccc4c4ccccc4c32)cc1 ; c1ccc2c(c1)ccc1ccc3cccc(-c4nc5ccccc5c5c4sc4ccccc45)c3c12 ; c1ccc(-c2nc(-c3ccccc3)nc(-c3cccc4ccc5ccc6cccc(-c7ccccc7)c6c5c34)n2)cc1 ; c1ccc(-c2nc(-c3ccccc3)nc(-c3cccc4ccc5ccc6cccc(-c7ccc8ccccc8c7)c6c5c34)n2)cc1]

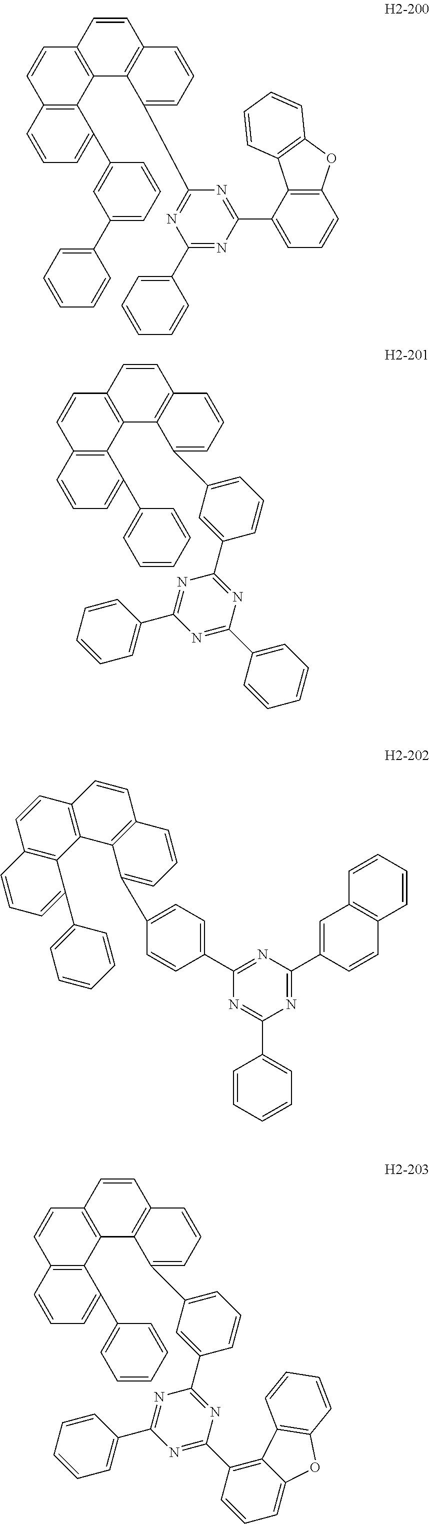

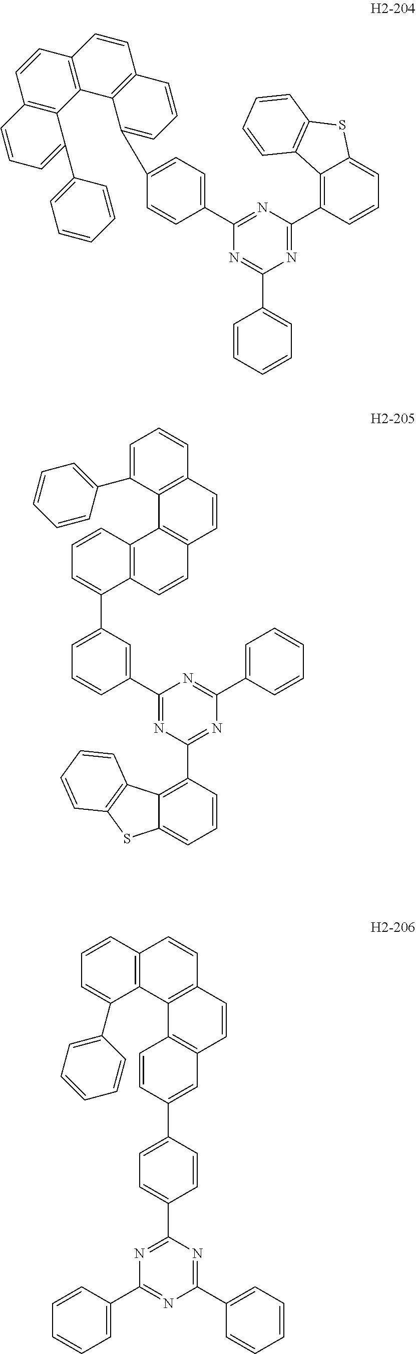 [SMILES: c1ccc(-c2nc(-c3ccc(-c4cccc5ccc6ccc7cccc(-c8ccccc8)c7c6c45)cc3)nc(-c3cccc4sc5ccccc5c34)n2)cc1 ; c1ccc(-c2nc(-c3cccc(-c4cccc5c4ccc4ccc6cccc(-c7ccccc7)c6c45)c3)nc(-c3cccc4sc5ccccc5c34)n2)cc1 ; c1ccc(-c2nc(-c3ccccc3)nc(-c3ccc(-c4ccc5c(ccc6ccc7cccc(-c8ccccc8)c7c65)c4)cc3)n2)cc1]

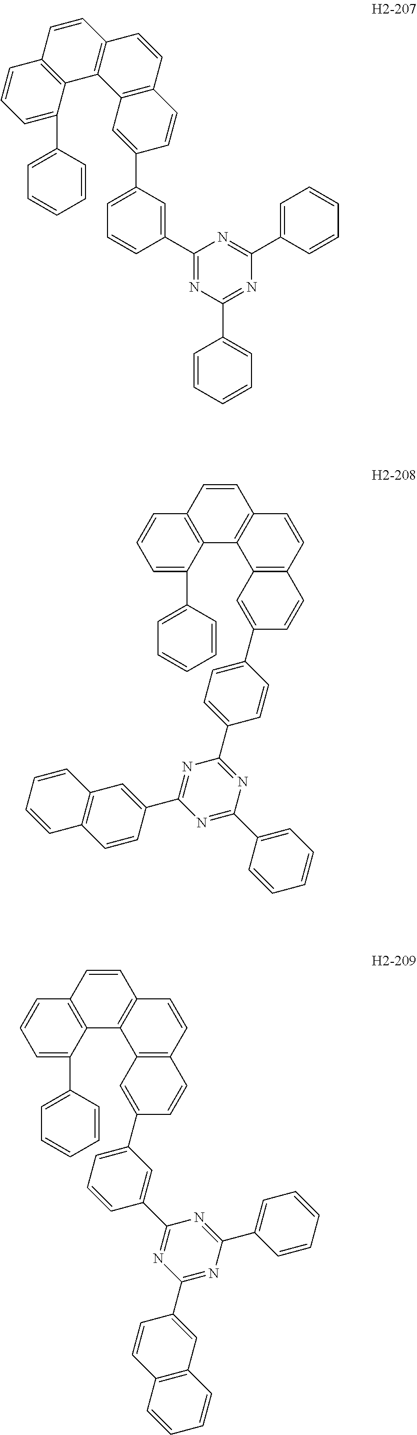 [SMILES: c1ccc(-c2nc(-c3ccccc3)nc(-c3cccc(-c4ccc5ccc6ccc7cccc(-c8ccccc8)c7c6c5c4)c3)n2)cc1 ; c1ccc(-c2nc(-c3ccc(-c4ccc5ccc6ccc7cccc(-c8ccccc8)c7c6c5c4)cc3)nc(-c3ccc4ccccc4c3)n2)cc1 ; c1ccc(-c2nc(-c3cccc(-c4ccc5ccc6ccc7cccc(-c8ccccc8)c7c6c5c4)c3)nc(-c3ccc4ccccc4c3)n2)cc1]

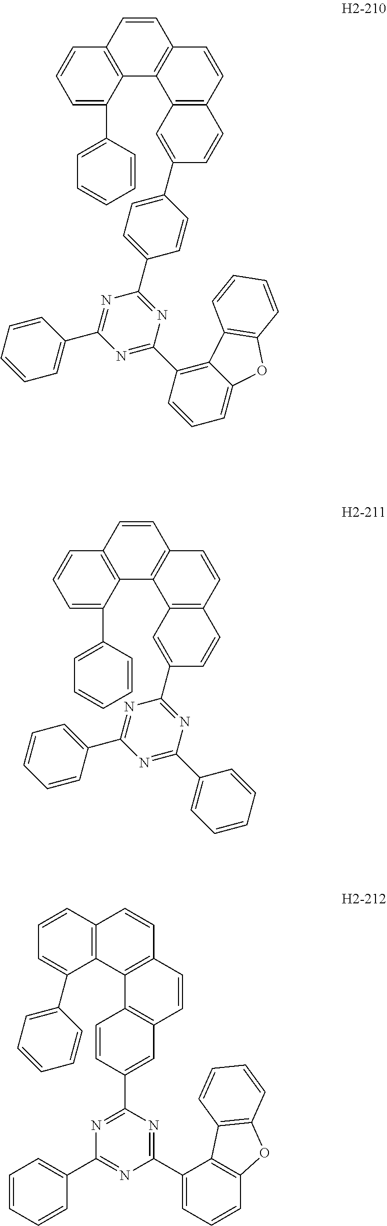 [SMILES: c1ccc(-c2nc(-c3ccc(-c4ccc5ccc6ccc7cccc(-c8ccccc8)c7c6c5c4)cc3)nc(-c3cccc4oc5ccccc5c34)n2)cc1 ; c1ccc(-c2nc(-c3ccccc3)nc(-c3ccc4ccc5ccc6cccc(-c7ccccc7)c6c5c4c3)n2)cc1 ; c1ccc(-c2nc(-c3ccc4c(ccc5ccc6cccc(-c7ccccc7)c6c54)c3)nc(-c3cccc4oc5ccccc5c34)n2)cc1]

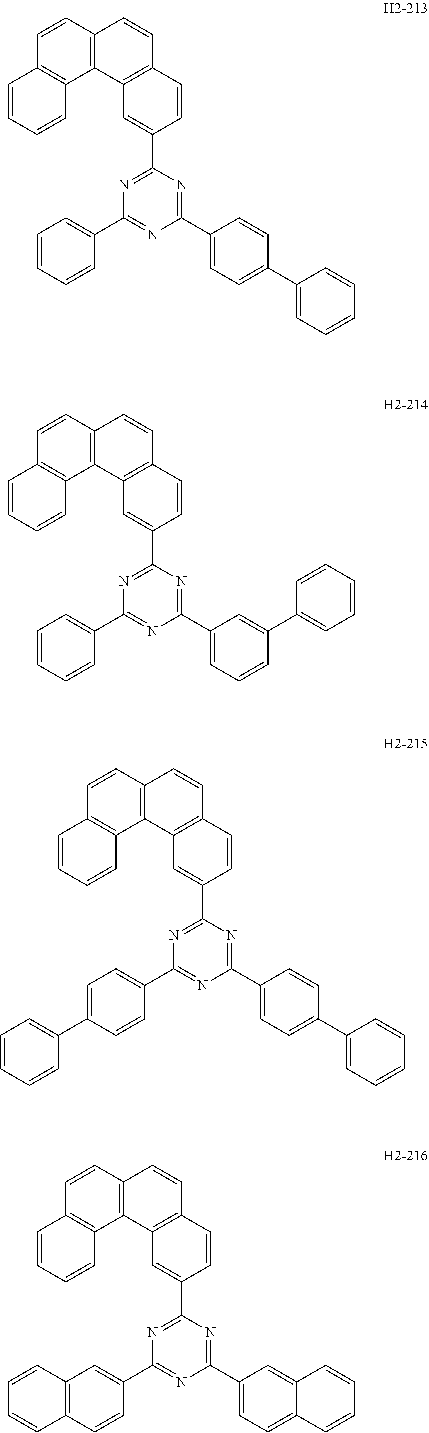

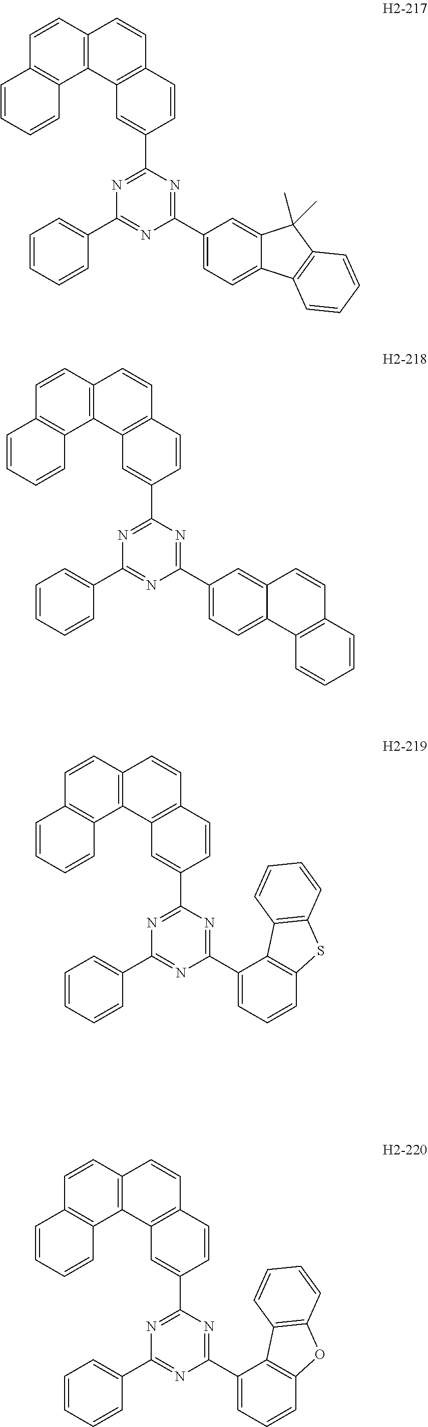 [SMILES: CC1(C)c2ccccc2-c2ccc(-c3nc(-c4ccccc4)nc(-c4ccc5ccc6ccc7ccccc7c6c5c4)n3)cc21 ; c1ccc(-c2nc(-c3ccc4c(ccc5ccccc54)c3)nc(-c3ccc4ccc5ccc6ccccc6c5c4c3)n2)cc1 ; c1ccc(-c2nc(-c3ccc4ccc5ccc6ccccc6c5c4c3)nc(-c3cccc4sc5ccccc5c34)n2)cc1 ; c1ccc(-c2nc(-c3ccc4ccc5ccc6ccccc6c5c4c3)nc(-c3cccc4oc5ccccc5c34)n2)cc1]

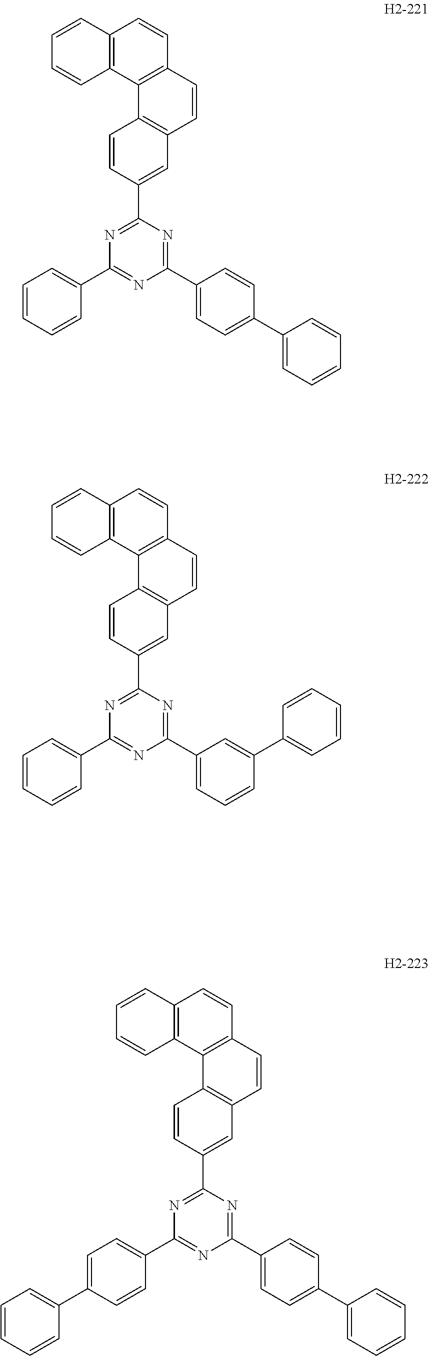 [SMILES: c1ccc(-c2ccc(-c3nc(-c4ccccc4)nc(-c4ccc5c(ccc6ccc7ccccc7c65)c4)n3)cc2)cc1 ; c1ccc(-c2cccc(-c3nc(-c4ccccc4)nc(-c4ccc5c(ccc6ccc7ccccc7c65)c4)n3)c2)cc1 ; c1ccc(-c2ccc(-c3nc(-c4ccc(-c5ccccc5)cc4)nc(-c4ccc5c(ccc6ccc7ccccc7c65)c4)n3)cc2)cc1]

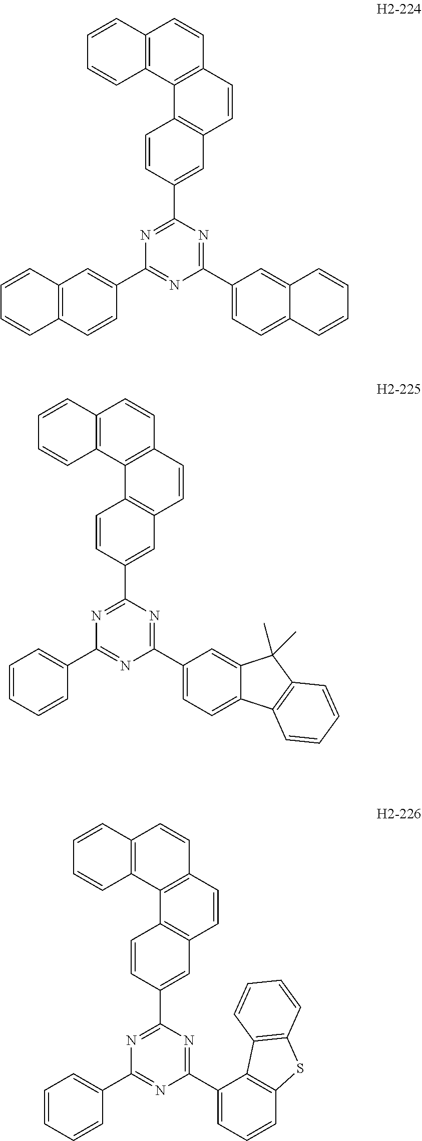 [SMILES: c1ccc2cc(-c3nc(-c4ccc5ccccc5c4)nc(-c4ccc5c(ccc6ccc7ccccc7c65)c4)n3)ccc2c1 ; CC1(C)c2ccccc2-c2ccc(-c3nc(-c4ccccc4)nc(-c4ccc5c(ccc6ccc7ccccc7c65)c4)n3)cc21 ; c1ccc(-c2nc(-c3ccc4c(ccc5ccc6ccccc6c54)c3)nc(-c3cccc4sc5ccccc5c34)n2)cc1]

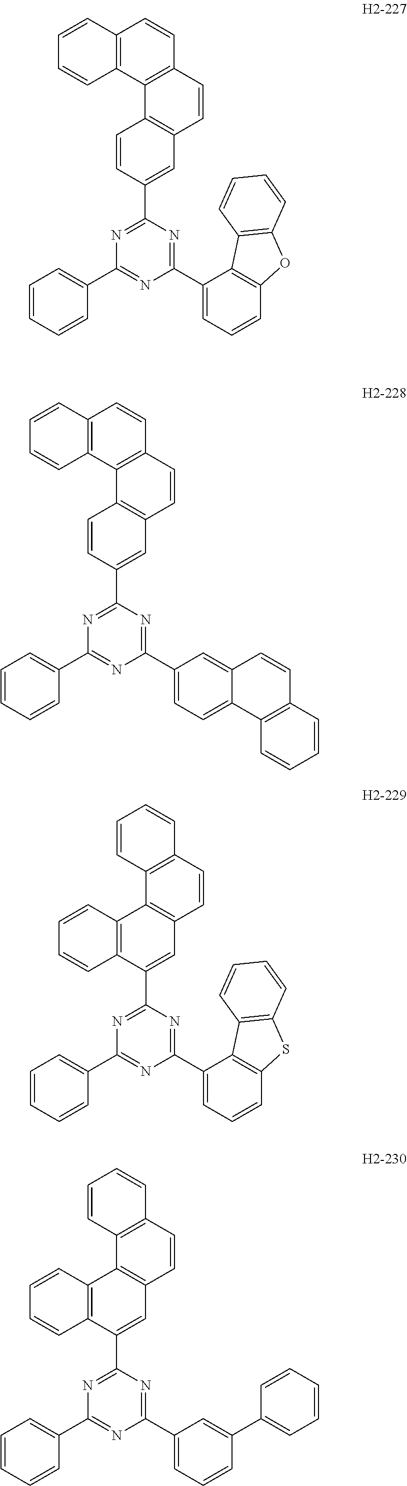 [SMILES: c1ccc(-c2nc(-c3ccc4c(ccc5ccc6ccccc6c54)c3)nc(-c3cccc4oc5ccccc5c34)n2)cc1 ; c1ccc(-c2nc(-c3ccc4c(ccc5ccccc54)c3)nc(-c3ccc4c(ccc5ccc6ccccc6c54)c3)n2)cc1 ; c1ccc(-c2nc(-c3cc4ccc5ccccc5c4c4ccccc34)nc(-c3cccc4sc5ccccc5c34)n2)cc1 ; c1ccc(-c2cccc(-c3nc(-c4ccccc4)nc(-c4cc5ccc6ccccc6c5c5ccccc45)n3)c2)cc1]

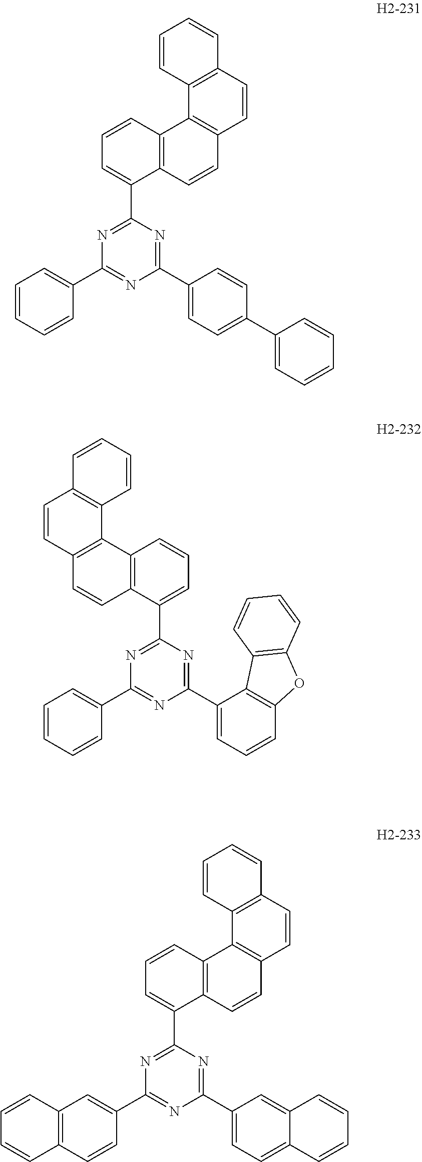 [SMILES: c1ccc(-c2ccc(-c3nc(-c4ccccc4)nc(-c4cccc5c4ccc4ccc6ccccc6c45)n3)cc2)cc1 ; c1ccc(-c2nc(-c3cccc4c3ccc3ccc5ccccc5c34)nc(-c3cccc4oc5ccccc5c34)n2)cc1 ; c1ccc2cc(-c3nc(-c4ccc5ccccc5c4)nc(-c4cccc5c4ccc4ccc6ccccc6c45)n3)ccc2c1]

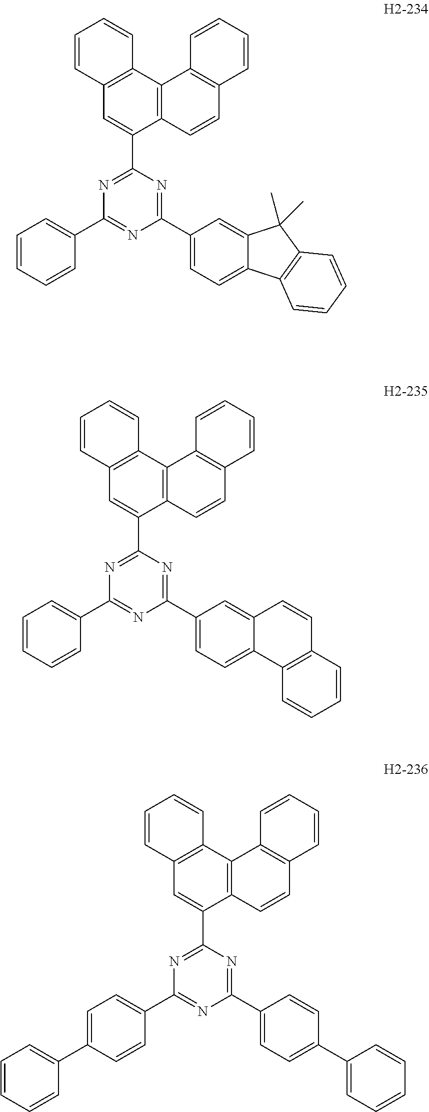 [SMILES: CC1(C)c2ccccc2-c2ccc(-c3nc(-c4ccccc4)nc(-c4cc5ccccc5c5c4ccc4ccccc45)n3)cc21 ; c1ccc(-c2nc(-c3ccc4c(ccc5ccccc54)c3)nc(-c3cc4ccccc4c4c3ccc3ccccc34)n2)cc1 ; c1ccc(-c2ccc(-c3nc(-c4ccc(-c5ccccc5)cc4)nc(-c4cc5ccccc5c5c4ccc4ccccc45)n3)cc2)cc1]

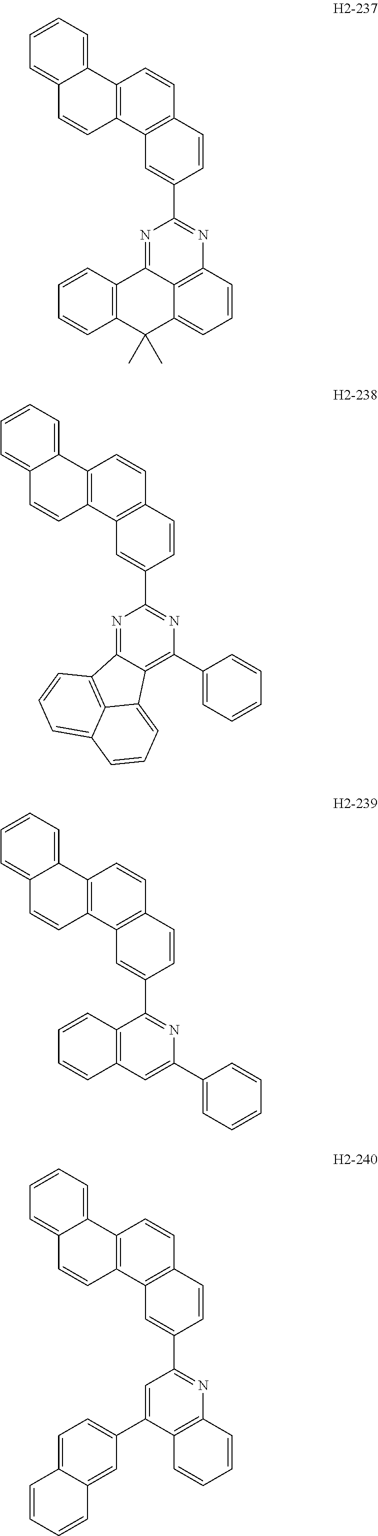 [SMILES: CC1(C)c2ccccc2-c2nc(-c3ccc4ccc5c6ccccc6ccc5c4c3)nc3cccc1c23 ; c1ccc(-c2nc(-c3ccc4ccc5c6ccccc6ccc5c4c3)nc3c2-c2cccc4cccc-3c24)cc1 ; c1ccc(-c2cc3ccccc3c(-c3ccc4ccc5c6ccccc6ccc5c4c3)n2)cc1 ; c1ccc2cc(-c3cc(-c4ccc5ccc6c7ccccc7ccc6c5c4)nc4ccccc34)ccc2c1]

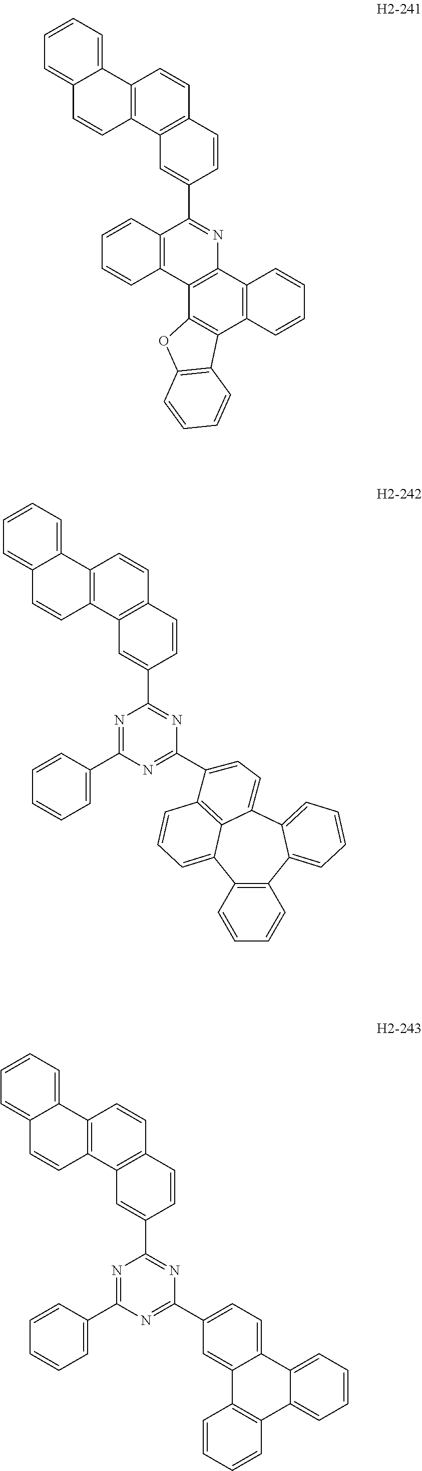 [SMILES: c1ccc2c(c1)ccc1c3cc(-c4nc5c6ccccc6c6c7ccccc7oc6c5c5ccccc45)ccc3ccc21 ; c1ccc(-c2nc(-c3ccc4ccc5c6ccccc6ccc5c4c3)nc(-c3ccc4c5c(cccc35)-c3ccccc3-c3ccccc3-4)n2)cc1 ; c1ccc(-c2nc(-c3ccc4ccc5c6ccccc6ccc5c4c3)nc(-c3ccc4c5ccccc5c5ccccc5c4c3)n2)cc1]

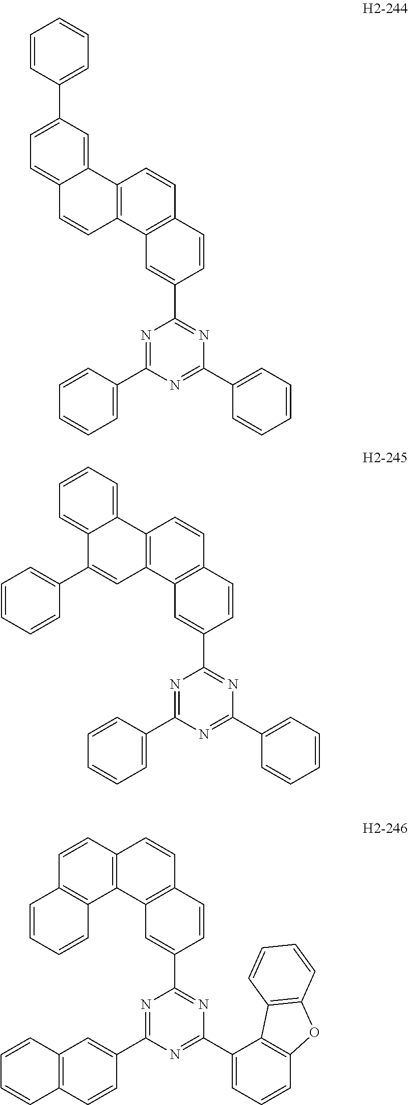

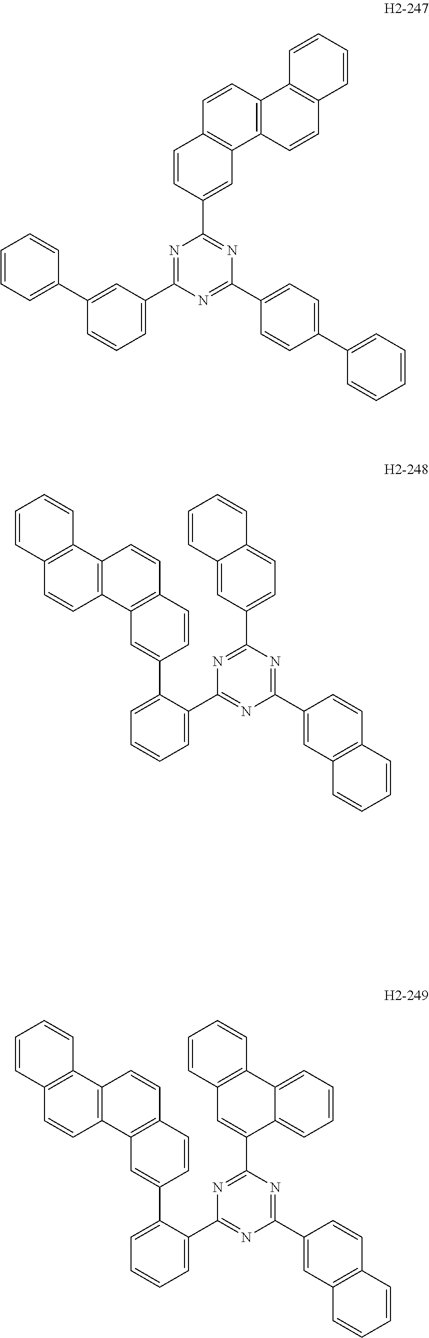 [SMILES: c1ccc(-c2ccc(-c3nc(-c4cccc(-c5ccccc5)c4)nc(-c4ccc5ccc6c7ccccc7ccc6c5c4)n3)cc2)cc1 ; c1ccc(-c2nc(-c3ccc4ccccc4c3)nc(-c3ccc4ccccc4c3)n2)c(-c2ccc3ccc4c5ccccc5ccc4c3c2)c1 ; c1ccc(-c2nc(-c3ccc4ccccc4c3)nc(-c3cc4ccccc4c4ccccc34)n2)c(-c2ccc3ccc4c5ccccc5ccc4c3c2)c1]

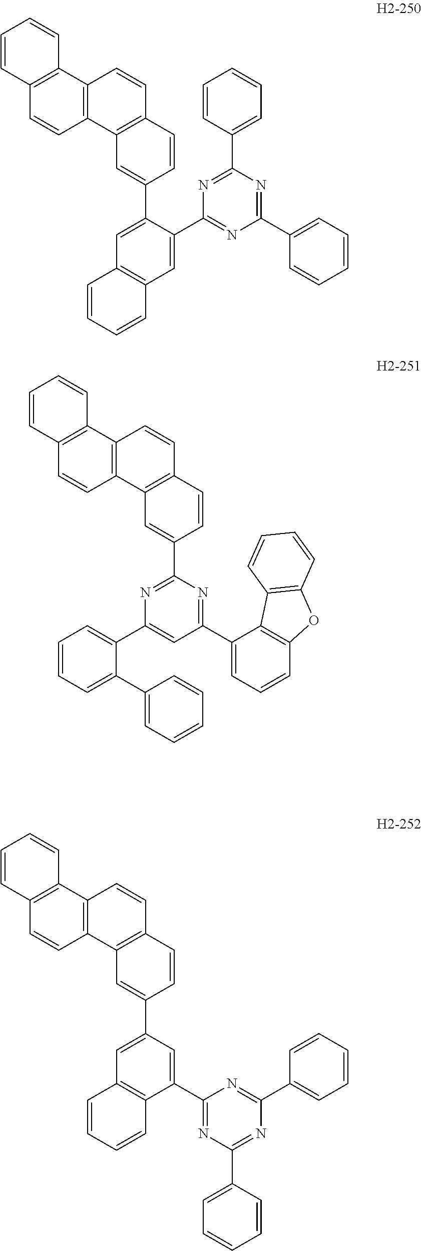 [SMILES: c1ccc(-c2nc(-c3ccccc3)nc(-c3cc4ccccc4cc3-c3ccc4ccc5c6ccccc6ccc5c4c3)n2)cc1 ; c1ccc(-c2ccccc2-c2cc(-c3cccc4oc5ccccc5c34)nc(-c3ccc4ccc5c6ccccc6ccc5c4c3)n2)cc1 ; c1ccc(-c2nc(-c3ccccc3)nc(-c3cc(-c4ccc5ccc6c7ccccc7ccc6c5c4)cc4ccccc34)n2)cc1]

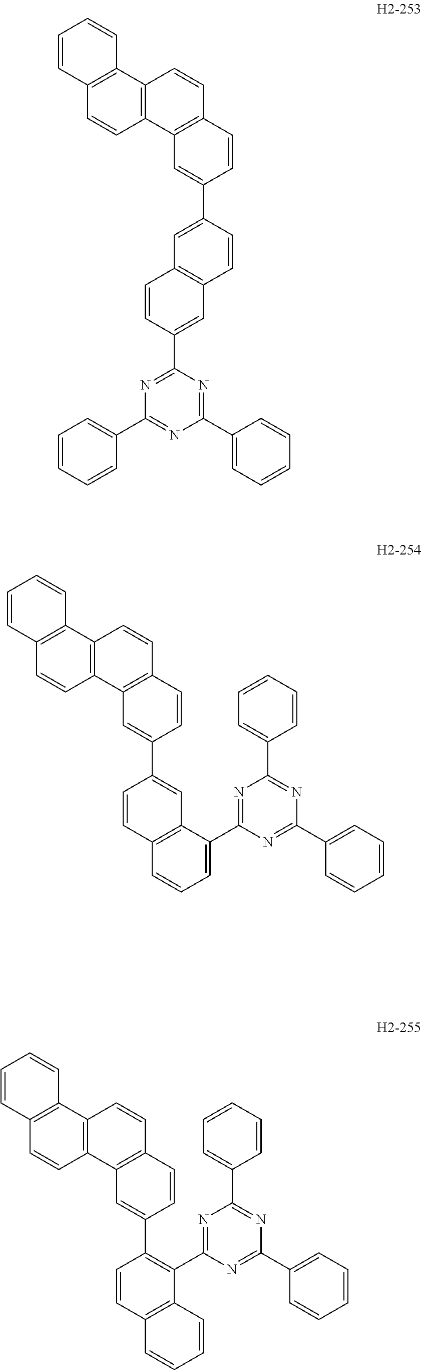 [SMILES: c1ccc(-c2nc(-c3ccccc3)nc(-c3ccc4cc(-c5ccc6ccc7c8ccccc8ccc7c6c5)ccc4c3)n2)cc1 ; c1ccc(-c2nc(-c3ccccc3)nc(-c3cccc4ccc(-c5ccc6ccc7c8ccccc8ccc7c6c5)cc34)n2)cc1 ; c1ccc(-c2nc(-c3ccccc3)nc(-c3c(-c4ccc5ccc6c7ccccc7ccc6c5c4)ccc4ccccc34)n2)cc1]

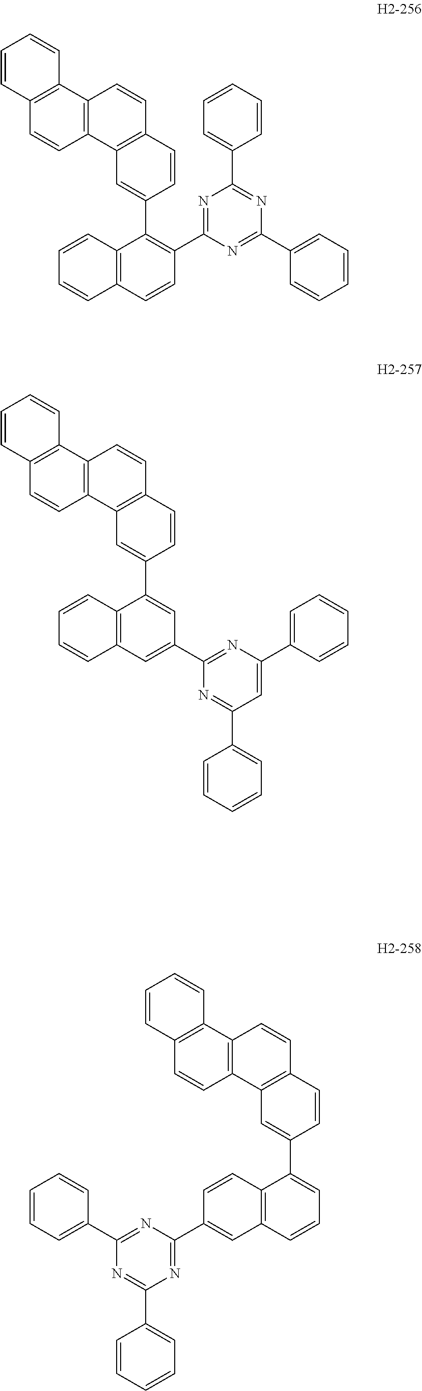 [SMILES: c1ccc(-c2nc(-c3ccccc3)nc(-c3ccc4ccccc4c3-c3ccc4ccc5c6ccccc6ccc5c4c3)n2)cc1 ; c1ccc(-c2cc(-c3ccccc3)nc(-c3cc(-c4ccc5ccc6c7ccccc7ccc6c5c4)c4ccccc4c3)n2)cc1 ; c1ccc(-c2nc(-c3ccccc3)nc(-c3ccc4c(-c5ccc6ccc7c8ccccc8ccc7c6c5)cccc4c3)n2)cc1]

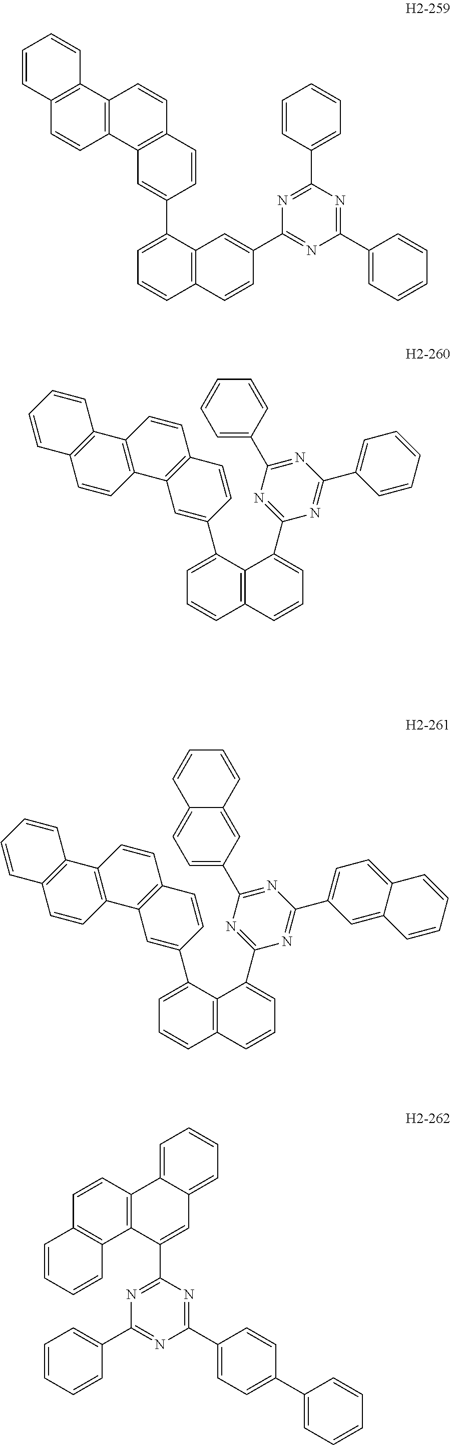 [SMILES: c1ccc(-c2nc(-c3ccccc3)nc(-c3ccc4cccc(-c5ccc6ccc7c8ccccc8ccc7c6c5)c4c3)n2)cc1 ; c1ccc(-c2nc(-c3ccccc3)nc(-c3cccc4cccc(-c5ccc6ccc7c8ccccc8ccc7c6c5)c34)n2)cc1 ; c1ccc2cc(-c3nc(-c4ccc5ccccc5c4)nc(-c4cccc5cccc(-c6ccc7ccc8c9ccccc9ccc8c7c6)c45)n3)ccc2c1 ; c1ccc(-c2ccc(-c3nc(-c4ccccc4)nc(-c4cc5ccccc5c5ccc6ccccc6c45)n3)cc2)cc1]

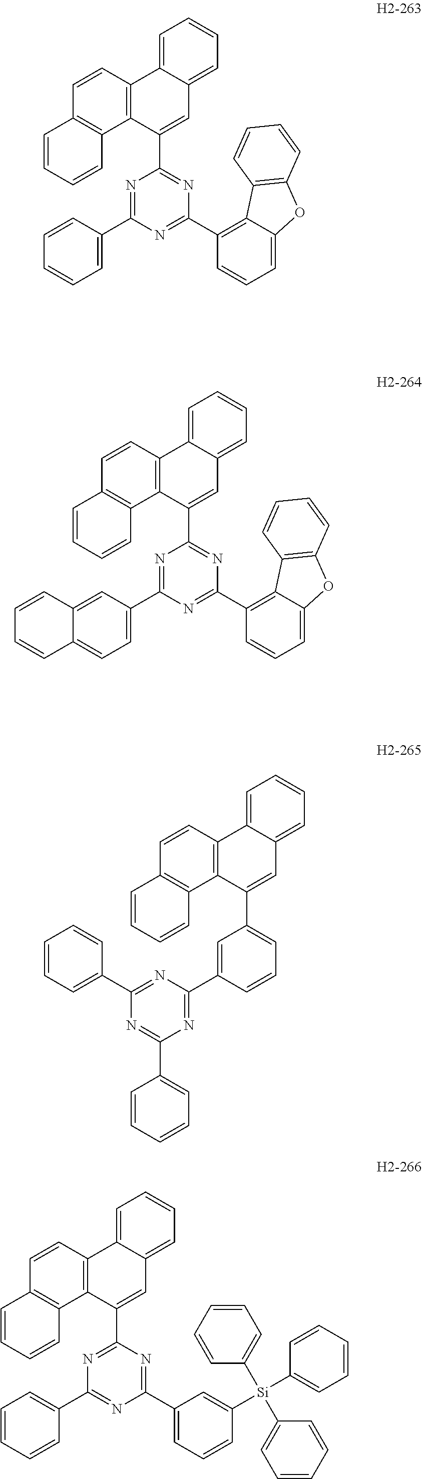 [SMILES: c1ccc(-c2nc(-c3cccc4oc5ccccc5c34)nc(-c3cc4ccccc4c4ccc5ccccc5c34)n2)cc1 ; c1ccc2cc(-c3nc(-c4cccc5oc6ccccc6c45)nc(-c4cc5ccccc5c5ccc6ccccc6c45)n3)ccc2c1 ; c1ccc(-c2nc(-c3ccccc3)nc(-c3cccc(-c4cc5ccccc5c5ccc6ccccc6c45)c3)n2)cc1 ; c1ccc(-c2nc(-c3cccc([Si](c4ccccc4)(c4ccccc4)c4ccccc4)c3)nc(-c3cc4ccccc4c4ccc5ccccc5c34)n2)cc1]

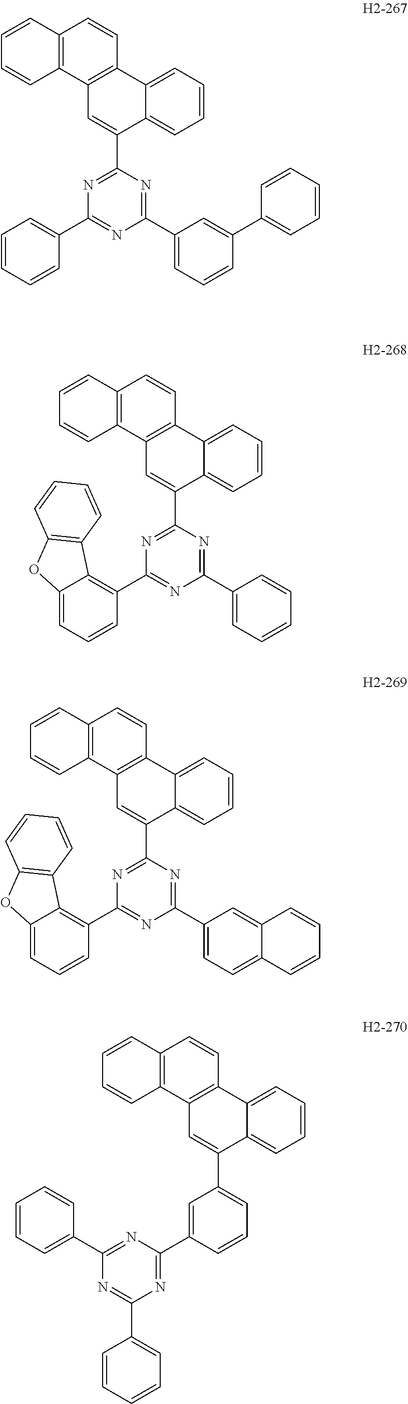 [SMILES: c1ccc(-c2cccc(-c3nc(-c4ccccc4)nc(-c4cc5c6ccccc6ccc5c5ccccc45)n3)c2)cc1 ; c1ccc(-c2nc(-c3cc4c5ccccc5ccc4c4ccccc34)nc(-c3cccc4oc5ccccc5c34)n2)cc1 ; c1ccc2cc(-c3nc(-c4cc5c6ccccc6ccc5c5ccccc45)nc(-c4cccc5oc6ccccc6c45)n3)ccc2c1 ; c1ccc(-c2nc(-c3ccccc3)nc(-c3cccc(-c4cc5c6ccccc6ccc5c5ccccc45)c3)n2)cc1]

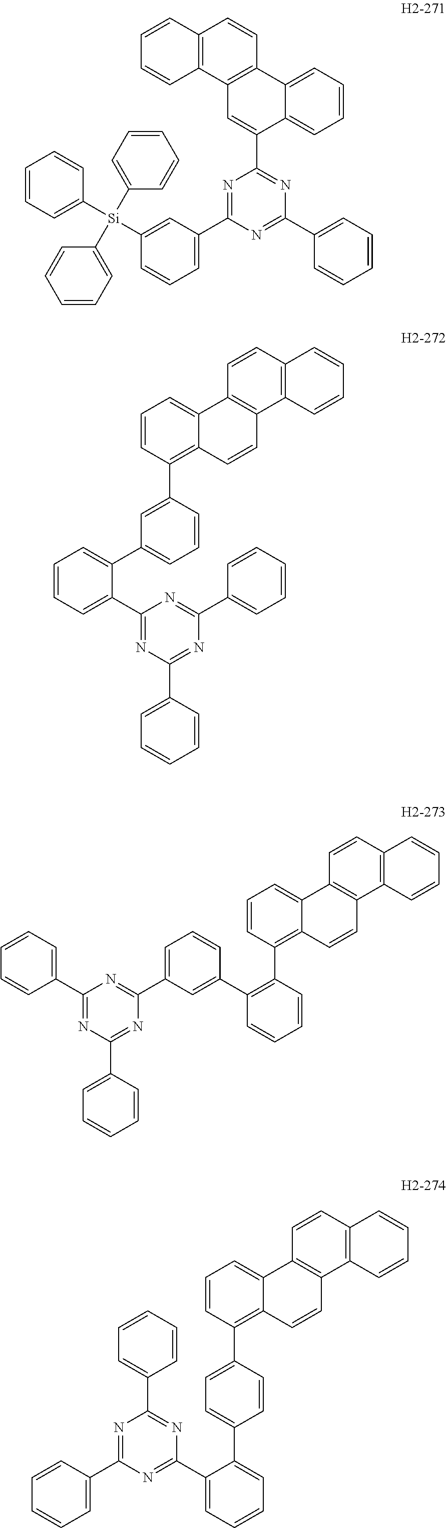 [SMILES: c1ccc(-c2nc(-c3cccc([Si](c4ccccc4)(c4ccccc4)c4ccccc4)c3)nc(-c3cc4c5ccccc5ccc4c4ccccc34)n2)cc1 ; c1ccc(-c2nc(-c3ccccc3)nc(-c3ccccc3-c3cccc(-c4cccc5c4ccc4c6ccccc6ccc54)c3)n2)cc1 ; c1ccc(-c2nc(-c3ccccc3)nc(-c3cccc(-c4ccccc4-c4cccc5c4ccc4c6ccccc6ccc54)c3)n2)cc1 ; c1ccc(-c2nc(-c3ccccc3)nc(-c3ccccc3-c3ccc(-c4cccc5c4ccc4c6ccccc6ccc54)cc3)n2)cc1]

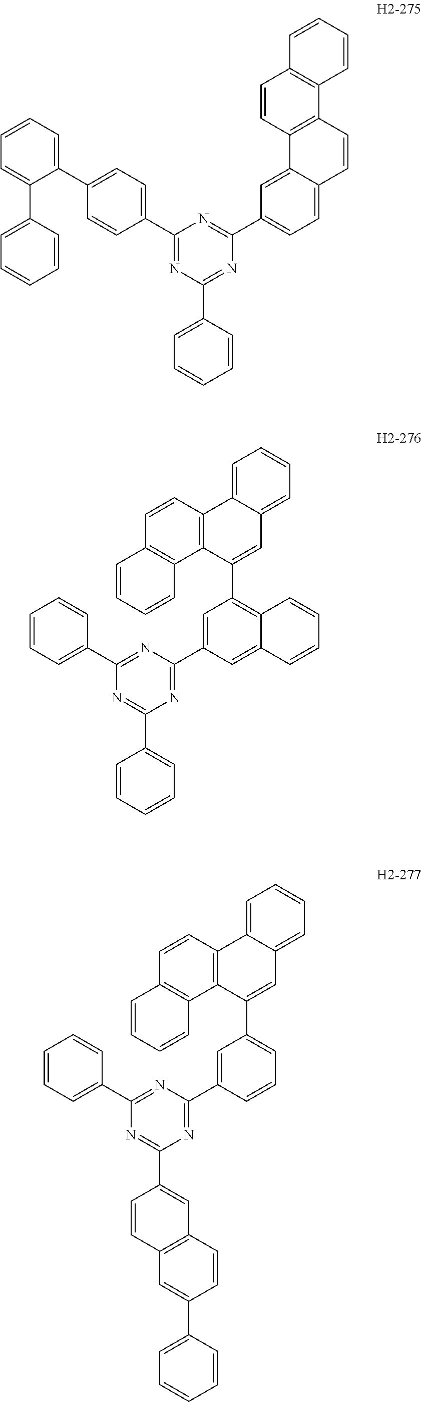 [SMILES: c1ccc(-c2nc(-c3ccc(-c4ccccc4-c4ccccc4)cc3)nc(-c3ccc4ccc5c6ccccc6ccc5c4c3)n2)cc1 ; c1ccc(-c2nc(-c3ccccc3)nc(-c3cc(-c4cc5ccccc5c5ccc6ccccc6c45)c4ccccc4c3)n2)cc1 ; c1ccc(-c2ccc3cc(-c4nc(-c5ccccc5)nc(-c5cccc(-c6cc7ccccc7c7ccc8ccccc8c67)c5)n4)ccc3c2)cc1]

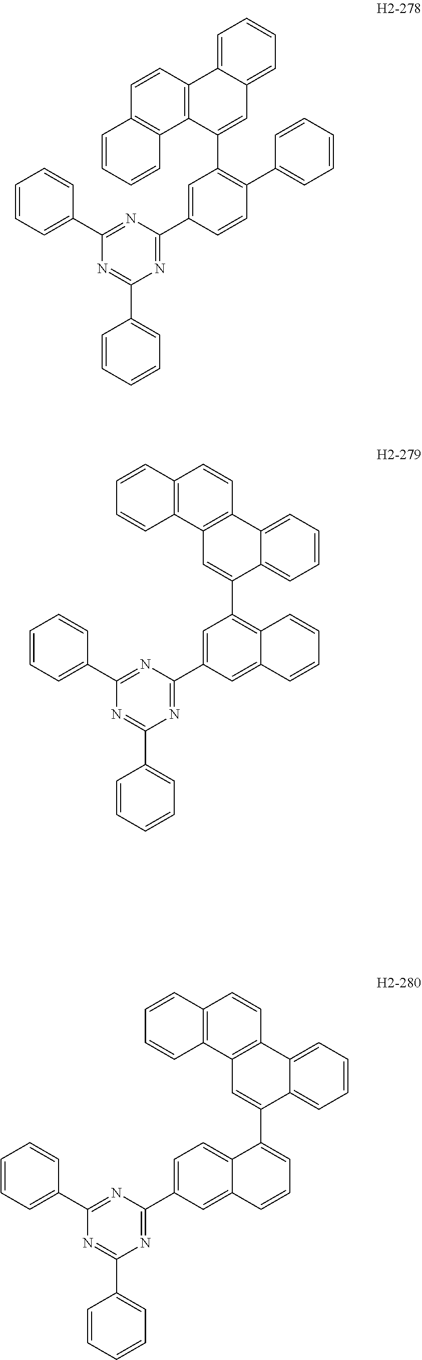 [SMILES: c1ccc(-c2nc(-c3ccccc3)nc(-c3ccc(-c4ccccc4)c(-c4cc5ccccc5c5ccc6ccccc6c45)c3)n2)cc1 ; c1ccc(-c2nc(-c3ccccc3)nc(-c3cc(-c4cc5c6ccccc6ccc5c5ccccc45)c4ccccc4c3)n2)cc1 ; c1ccc(-c2nc(-c3ccccc3)nc(-c3ccc4c(-c5cc6c7ccccc7ccc6c6ccccc56)cccc4c3)n2)cc1]

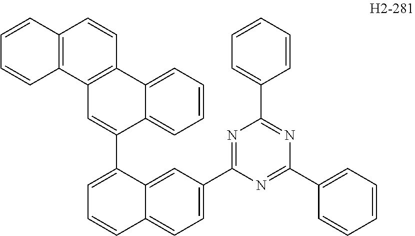 [SMILES: c1ccc(-c2nc(-c3ccccc3)nc(-c3ccc4cccc(-c5cc6c7ccccc7ccc6c6ccccc56)c4c3)n2)cc1]

The combination of at least one of compounds H1-1 to H1-127 and at least one of compounds H2-1 to H2-281 may be used in an organic electroluminescent device.

According to one embodiment of the present disclosure, the present disclosure may provide a compound represented by formula 1 or a compound represented by formula 2. Specifically, the present disclosure may provide at least one compound selected from the group consisting of compounds H1-1 to H1-127 and compounds H2-1 to H2-281.

The compound represented by formula 1 according to the present disclosure may be prepared by a synthetic method known to one skilled in the art. For example, the compound represented by formula 1 can be prepared by referring to Korean Patent Application Laid-Open Nos. 2017-0022865 (published on Mar. 2, 2017), and 2018-0099487 (published on Sep. 5, 2018), but is not limited thereto.

The compound represented by formula 2 according to the present disclosure may be produced by a synthetic method known to one skilled in the art, and for example, can be prepared by referring to the following reaction schemes, but are not limited thereto:

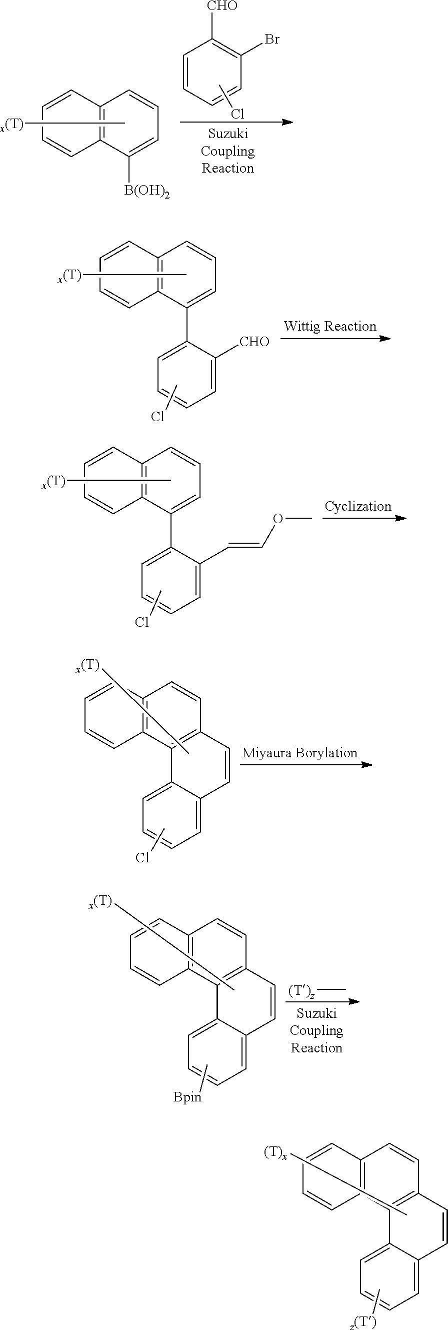

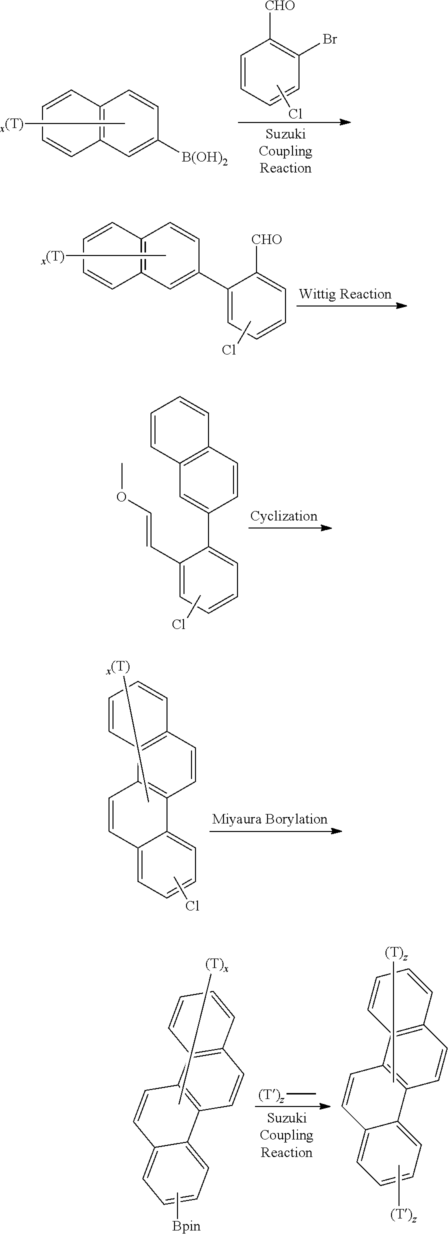

In reaction schemes 1 and 2, the definitions of T and T′, each independently, are the same as those of T₁ to T₁₄ in formula 1; x represents an integer of 1 to 7; z represents an integer of 1 to 4; where if each of x and z is an integer of 2 or more, each of T, and each of T′ may be the same or different.

Although illustrative synthesis examples of the compounds represented by formula 2 of the present disclosure are described above, one skilled in the art will be able to readily understand that all of them are based on a Buchwald-Hartwig cross-coupling reaction, an N-arylation reaction, a H-mont-mediated etherification reaction, a Miyaura borylation reaction, a Suzuki cross-coupling reaction, an Intramolecular acid-induced cyclization reaction, a Pd(II)-catalyzed oxidative cyclization reaction, a Grignard reaction, a Heck reaction, a Cyclic Dehydration reaction, an SN substitution reaction, an SN₂ substitution reaction, a Phosphine-mediated reductive cyclization reaction, etc., and the reactions above proceed even when substituents which are defined in formula 2 above, but are not specified in the specific synthesis examples, are bonded.

The organic electroluminescent device according to the present disclosure comprises an anode, a cathode, and at least one organic layer between the anode and the cathode. The organic layer may comprise a plurality of organic electroluminescent materials in which the compound represented by formula 1 is comprised as a first organic electroluminescent material, and the compound represented by formula 2 is comprised as a second organic electroluminescent material. According to one embodiment of the present disclosure, the organic electroluminescent device comprises an anode, a cathode, and at least one light-emitting layer between the anode and the cathode, and the light-emitting layer(s) comprises a compound(s) represented by formula 1, and a compound(s) represented by formula 2.

The electrode may be a transflective electrode or a reflective electrode, and may be a top emission type, a bottom emission type, or a both-sides emission type, depending on the materials. The hole injection layer may be further doped with a p-dopant, and the electron injection layer may be further doped with an n-dopant.

Herein, the light-emitting layer comprises a host and a dopant. The host comprises a plurality of host materials. The compound represented by formula 1 may be comprised as a first host compound of the plurality of host materials, and the compound represented by formula 2 may be comprised as a second host compound of the plurality of host materials. The weight ratio of the first host compound to the second host compound is in the range of about 1:99 to about 99:1, preferably about 10:90 to about 90:10, more preferably about 30:70 to about 70:30, even more preferably about 40:60 to about 60:40, and still more preferably about 50:50. When two or more materials are included in one layer, they may be mixture-evaporated to form a layer, or may be separately co-evaporated at the same time to form a layer.

The light-emitting layer is a layer from which light is emitted, and can be a single layer or a multi-layer in which two or more layers are stacked. In the plurality of host materials according to the present disclosure, the first and second host materials may both be comprised in one layer, or may be respectively comprised in different light-emitting layers. According to one embodiment of the present disclosure, the doping concentration of the dopant compound with respect to the host compound in the light-emitting layer is less than about 20 wt %.

The organic electroluminescent device of the present disclosure may further comprise at least one layer selected from a hole injection layer, a hole transport layer, a hole auxiliary layer, a light-emitting auxiliary layer, an electron transport layer, an electron injection layer, an interlayer, an electron buffer layer, a hole blocking layer, and an electron blocking layer. According to one embodiment of the present disclosure, the organic electroluminescent device may further comprise amine-based compounds in addition to the plurality of host materials of the present disclosure as at least one of a hole injection material, a hole transport material, a hole auxiliary material, a light-emitting material, a light-emitting auxiliary material, and an electron blocking material. Also, according to one embodiment of the present disclosure, the organic electroluminescent device of the present disclosure may further comprise azine-based compounds in addition to the plurality of host materials of the present disclosure as at least one of an electron transport material, an electron injection material, an electron buffer material, and a hole blocking material.

The dopant comprised in the organic electroluminescent device according to the present disclosure may be at least one phosphorescent or fluorescent dopant, preferably at least one phosphorescent dopant. The phosphorescent dopant materials applied to the organic electroluminescent device according to the present disclosure are not particularly limited, but may be selected from metallated complex compounds of iridium (Ir), osmium (Os), copper (Cu), and platinum (Pt), may be preferably selected from ortho-metallated complex compounds of iridium (Ir), osmium (Os), copper (Cu), and platinum (Pt), and may be more preferably an ortho-metallated iridium complex compound.

The dopant comprised in the organic electroluminescent device of the present disclosure may include the compound represented by the following formula 101, but is not limited thereto.

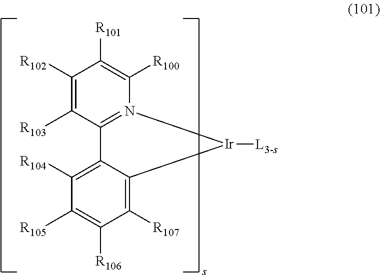

In formula 101, L is selected from the following structures 1 and 2:

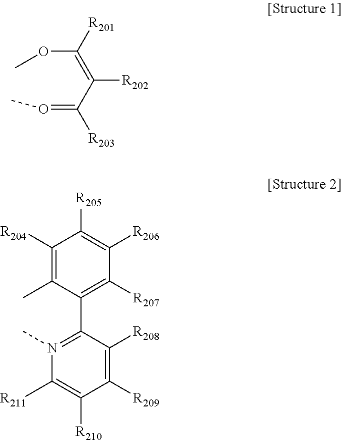

R₁₀₀ to R₁₀₃, each independently, represent hydrogen, deuterium, a halogen, a (C1-C30)alkyl unsubstituted or substituted with deuterium and/or a halogen(s), a substituted or unsubstituted (C3-C30)cycloalkyl, a substituted or unsubstituted (C6-C30)aryl, a cyano, a substituted or unsubstituted (3- to 30-membered)heteroaryl, or a substituted or unsubstituted (C1-C30)alkoxy; or may be linked to an adjacent substituent to form a ring(s), e.g., a substituted or unsubstituted, quinoline, isoquinoline, benzofuropyridine, benzothienopyridine, indenopyridine, benzofuroquinoline, benzothienoquinoline, or indenoquinoline ring, together with pyridine;

R₁₀₄ to R₁₀₇, each independently, represent hydrogen, deuterium, a halogen, a (C1-C30)alkyl unsubstituted or substituted with deuterium and/or a halogen(s), a substituted or unsubstituted (C3-C30)cycloalkyl, a substituted or unsubstituted (C6-C30)aryl, a substituted or unsubstituted (3- to 30-membered)heteroaryl, a cyano, or a substituted or unsubstituted (C1-C30)alkoxy; or may be linked to an adjacent substituent to form a ring(s), e.g., a substituted or unsubstituted, naphthalene, fluorene, dibenzothiophene, dibenzofuran, indenopyridine, benzofuropyridine or benzothienopyridine ring, together with benzene;

R₂₀₁ to R₂₁₁, each independently, represent hydrogen, deuterium, a halogen, a (C1-C30)alkyl unsubstituted or substituted with deuterium and/or a halogen(s), a substituted or unsubstituted (C3-C30)cycloalkyl, or a substituted or unsubstituted (C6-C30)aryl; or may be linked to an adjacent substituent to form a ring(s); and

s represents an integer of 1 to 3.

The specific examples of the dopant compound are as follows, but are not limited thereto.

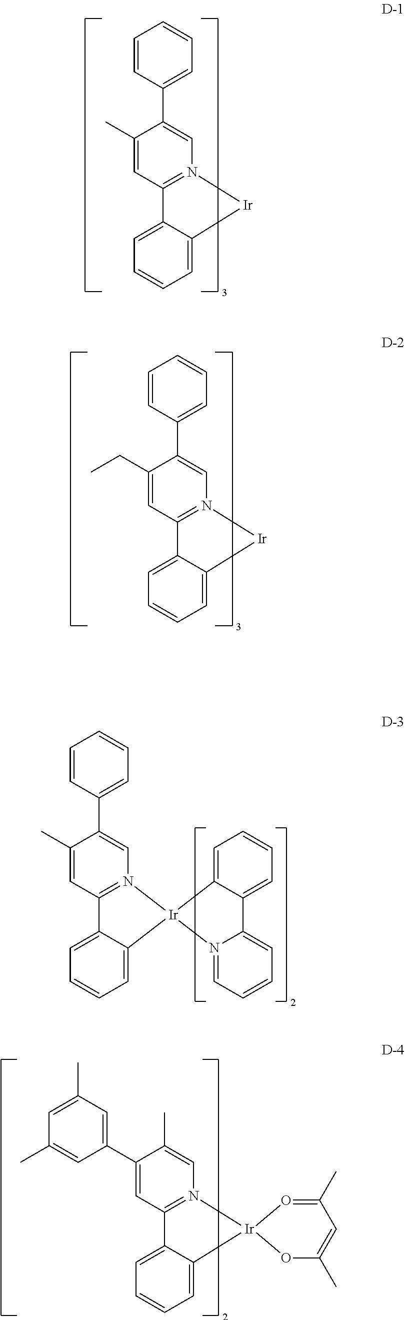

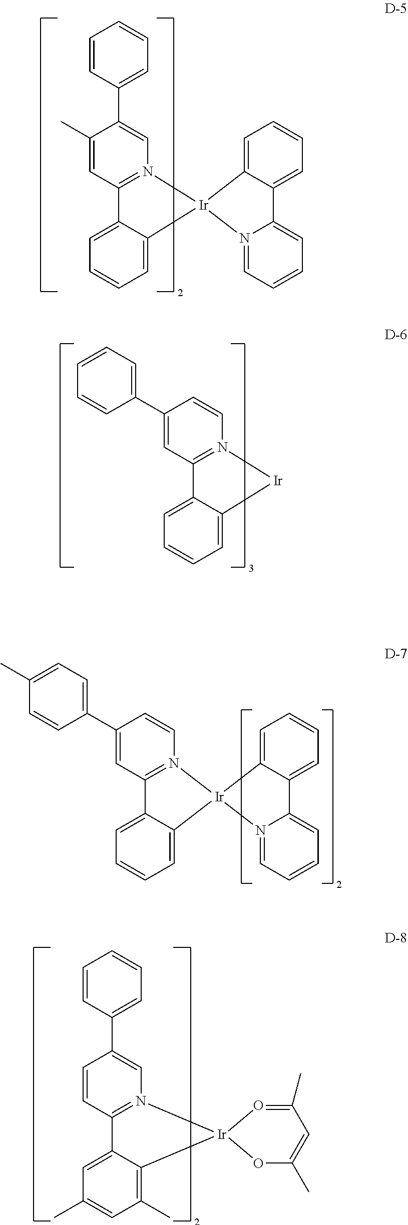

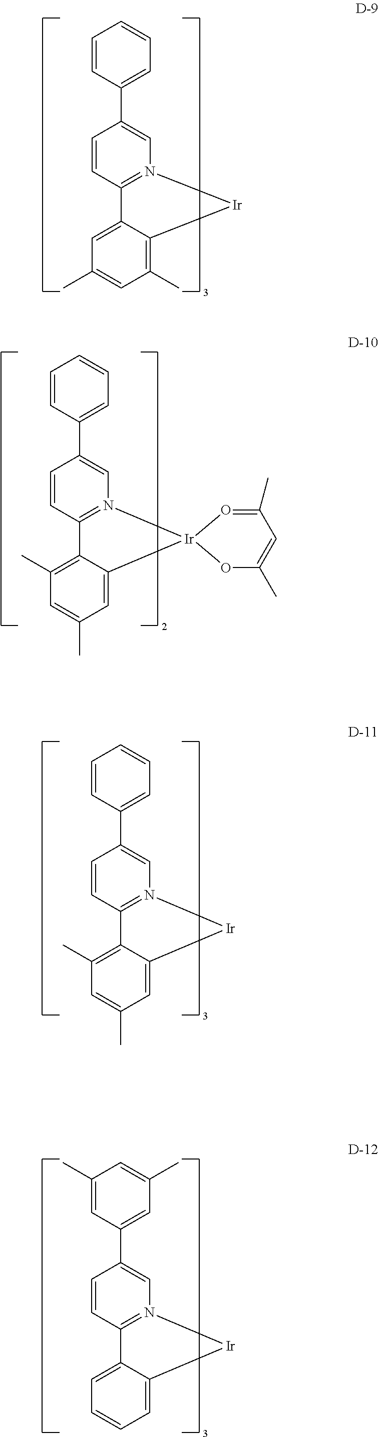

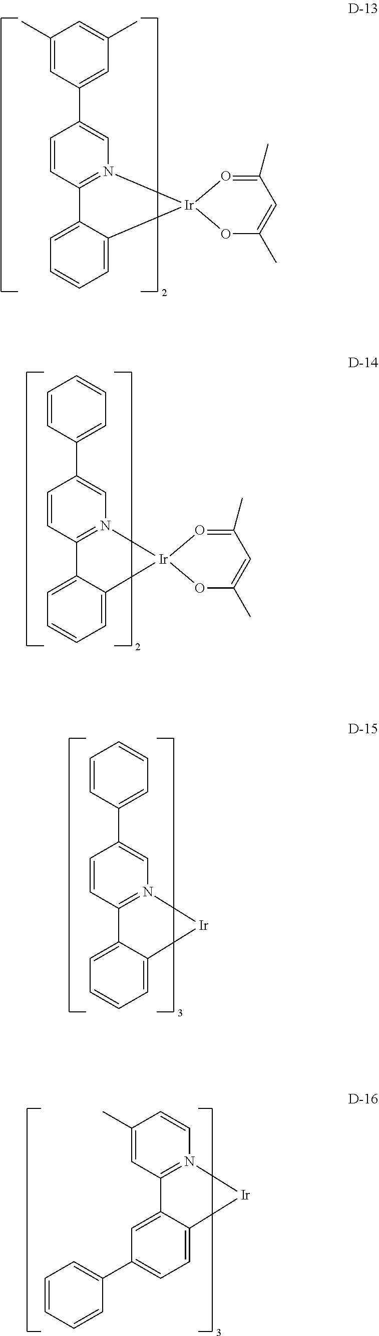

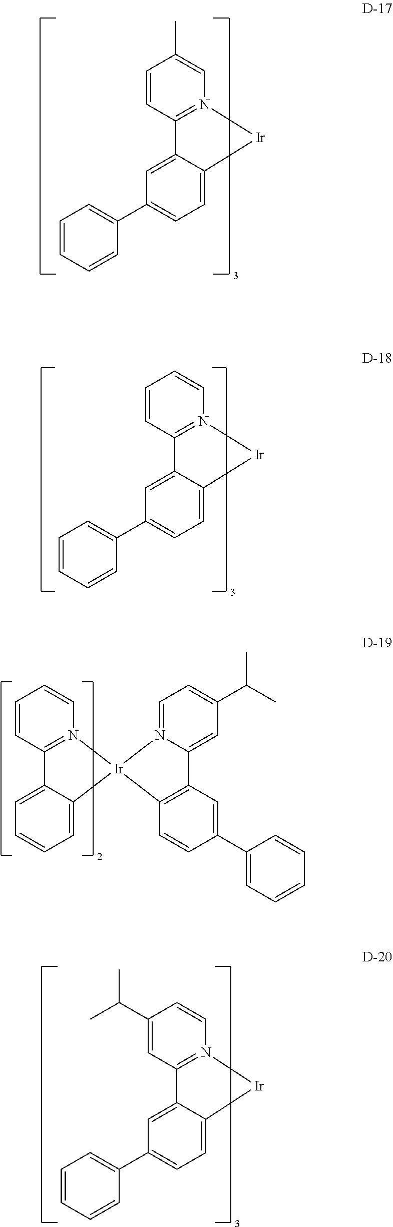

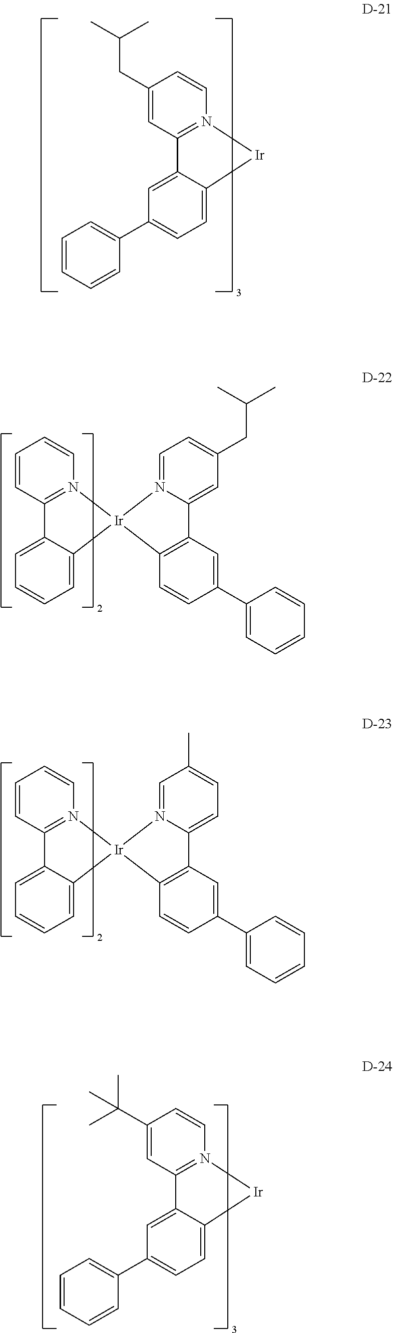

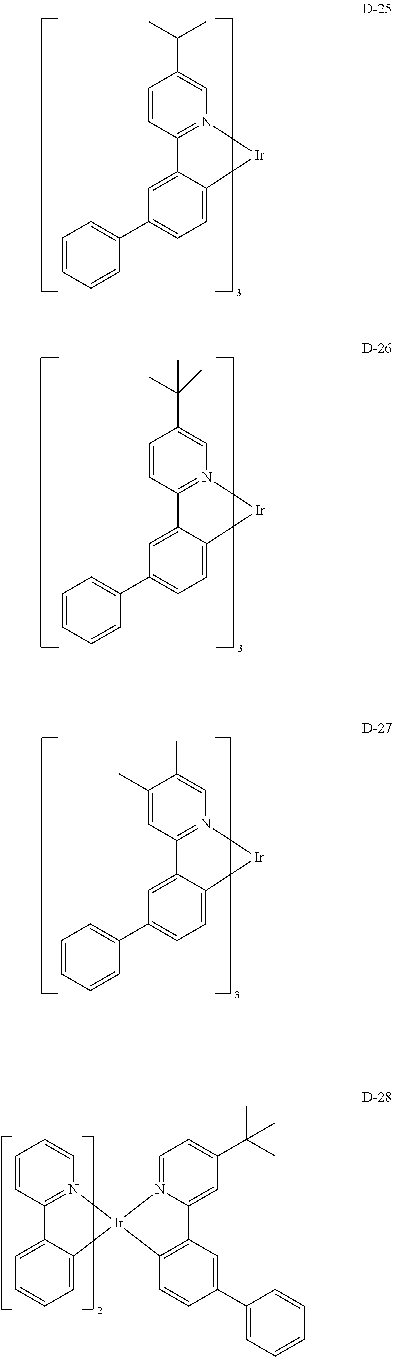

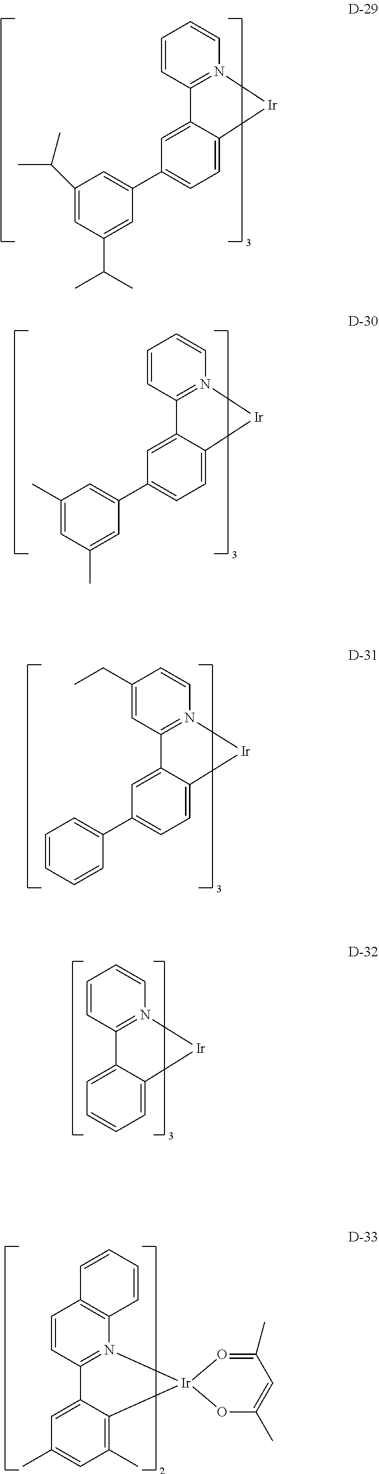

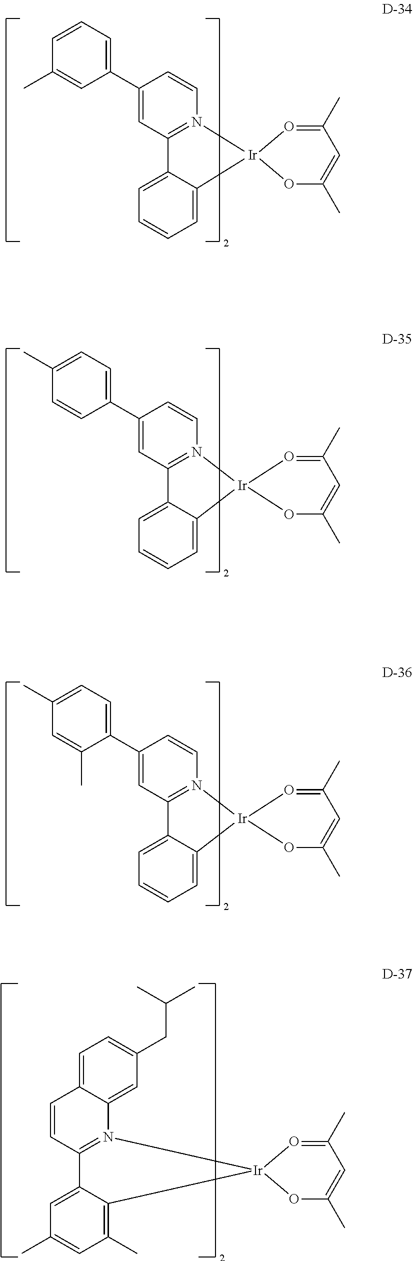

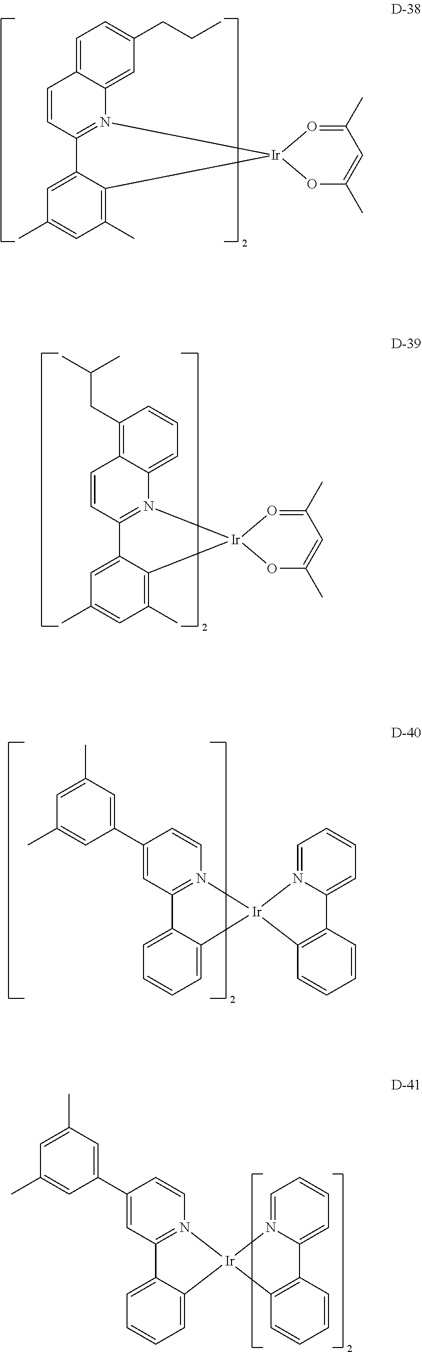

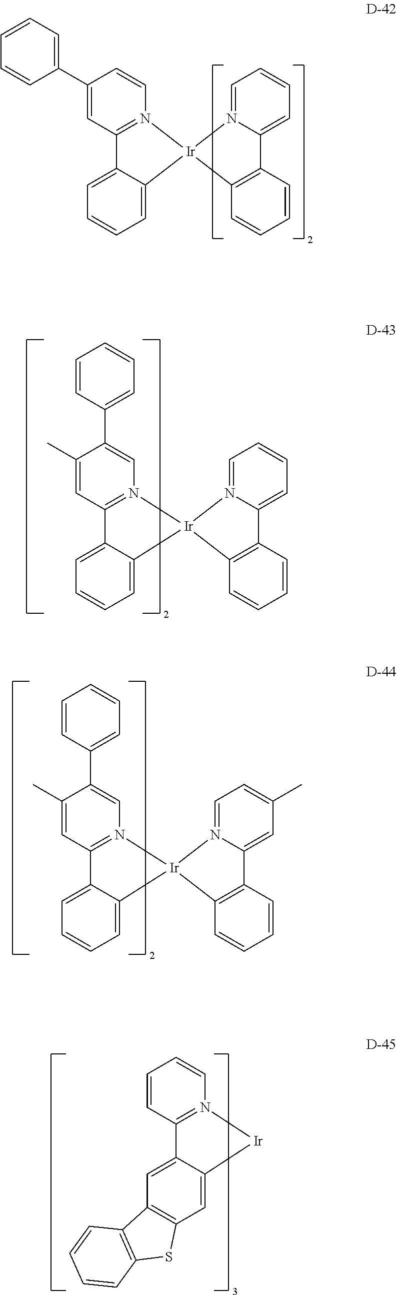

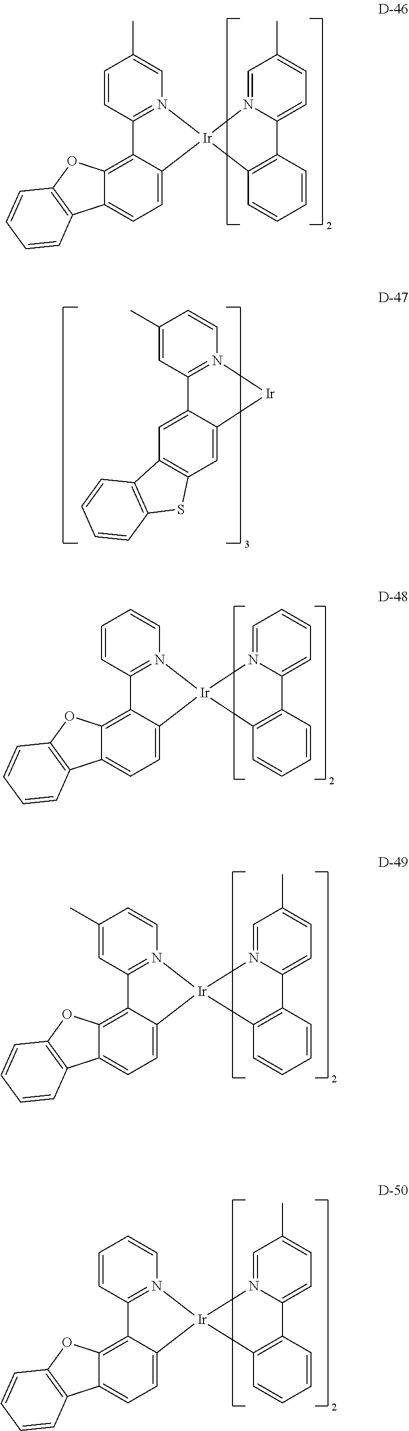

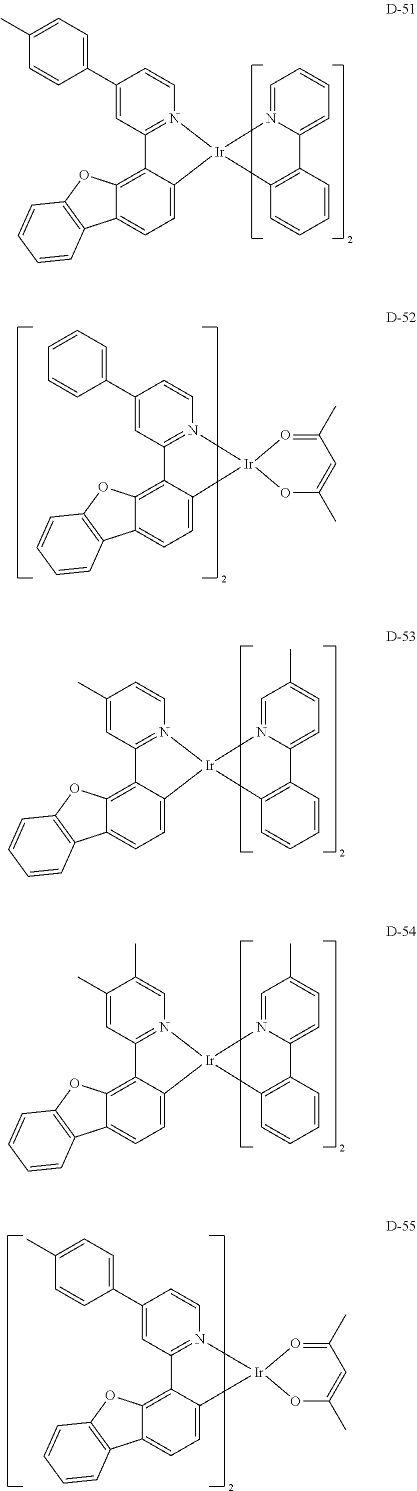

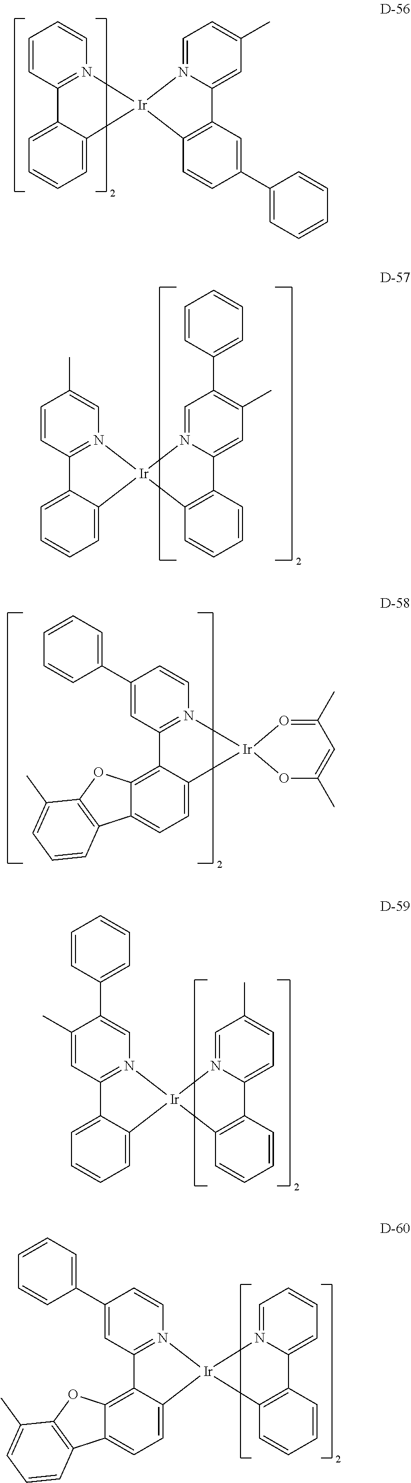

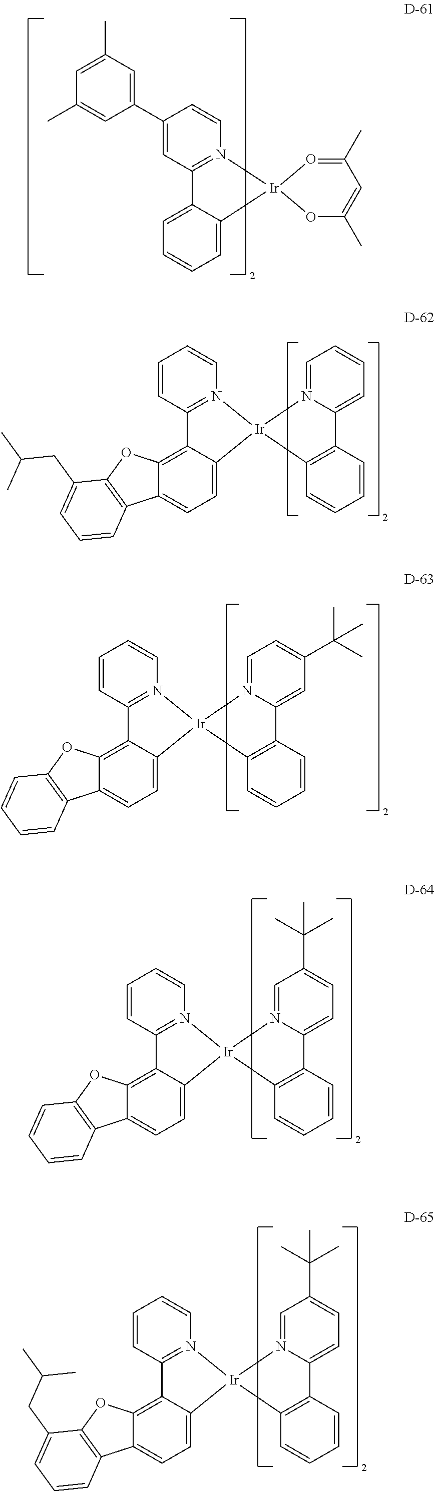

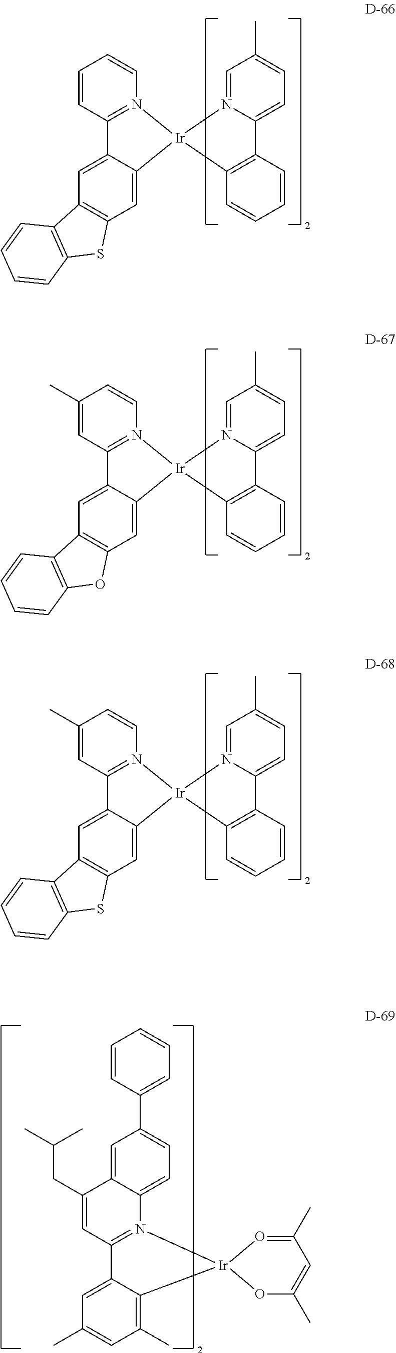

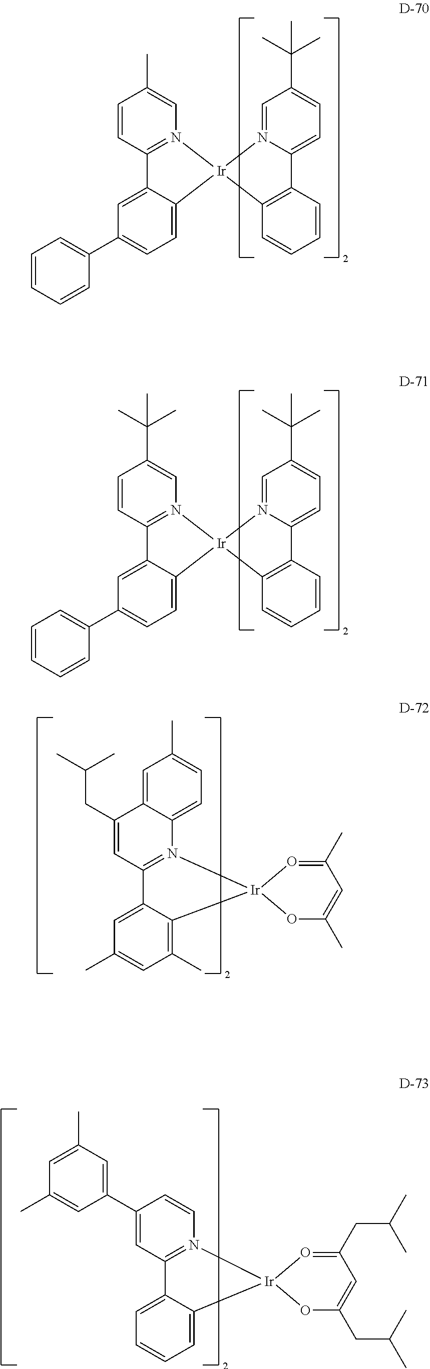

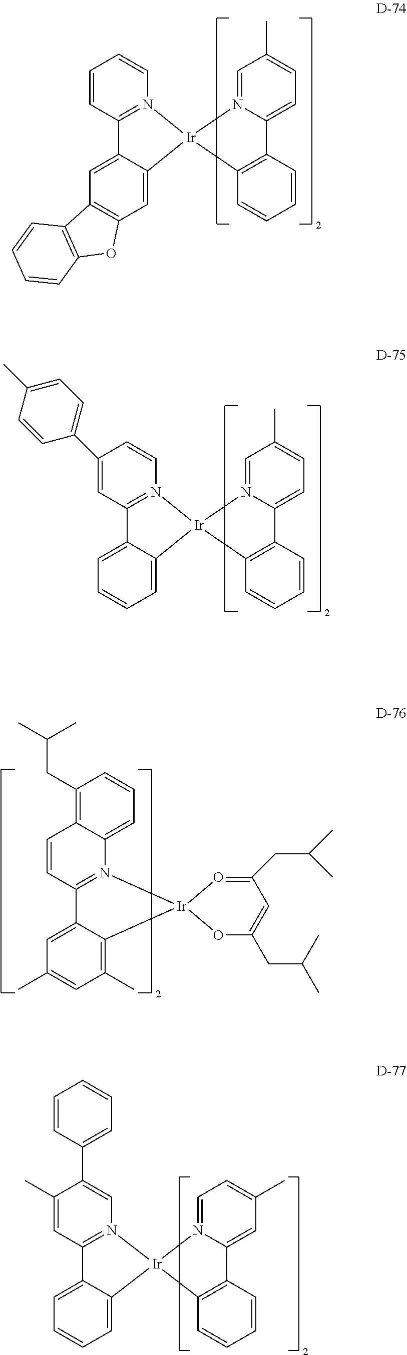

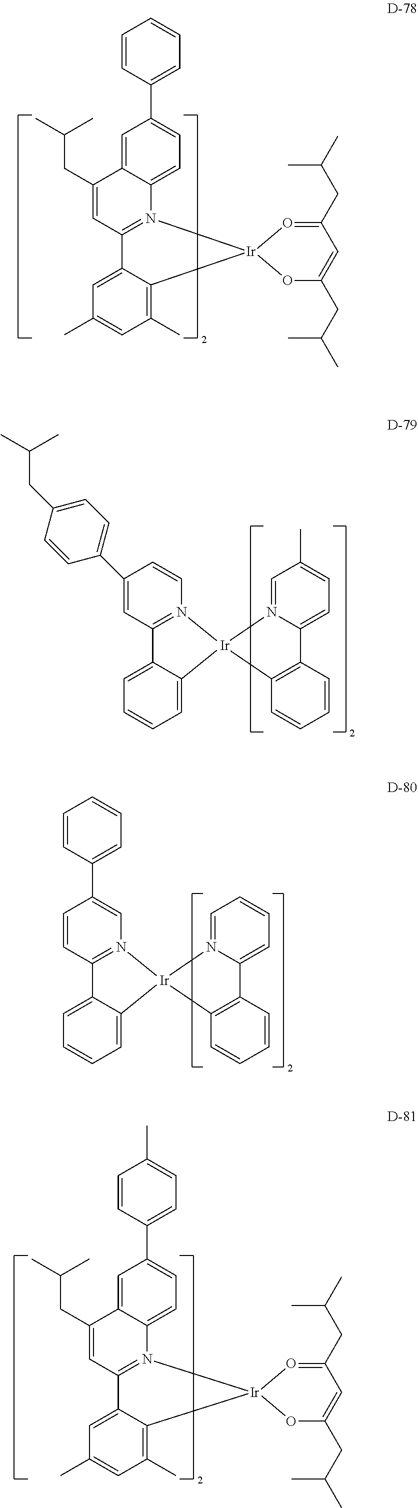

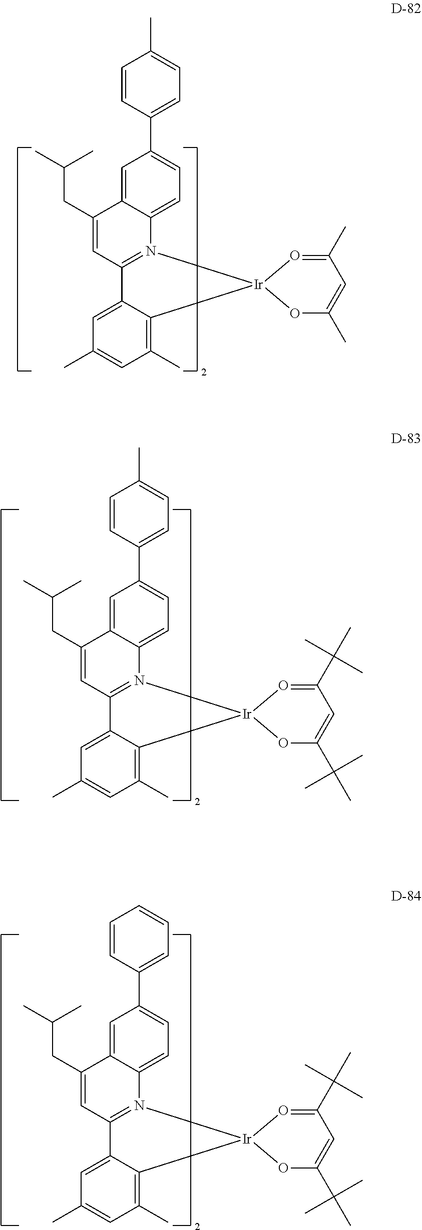

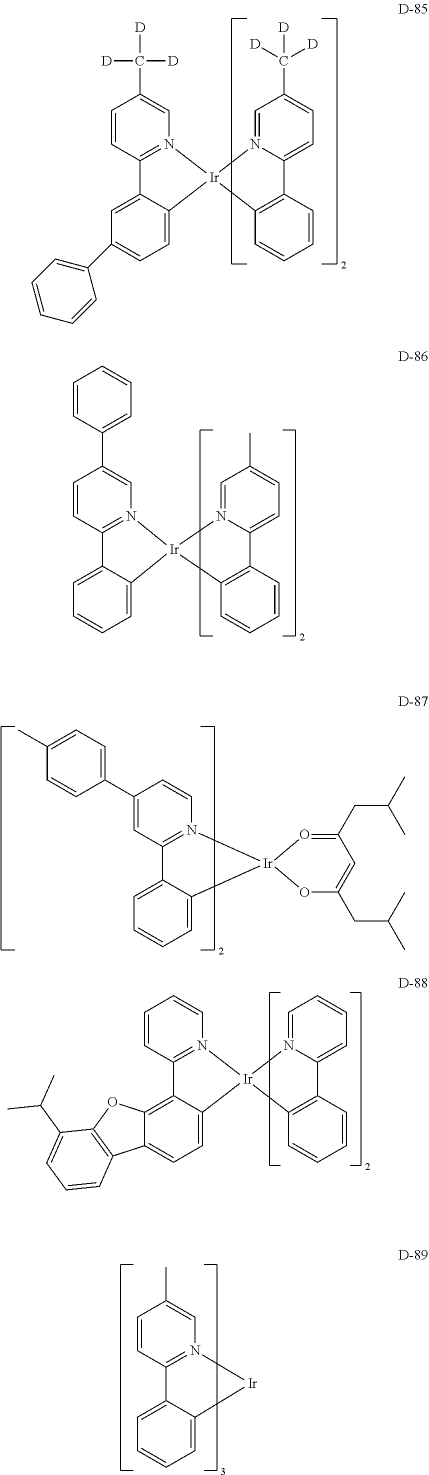

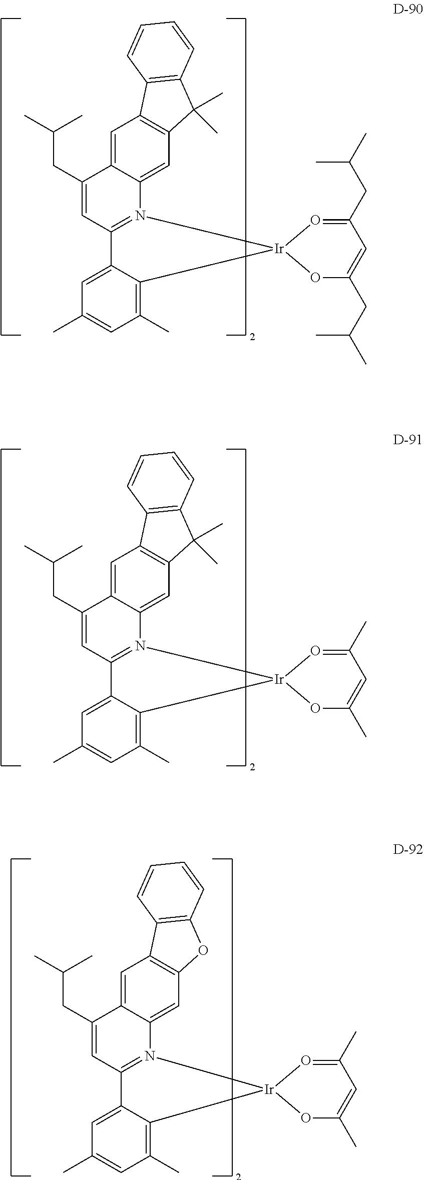

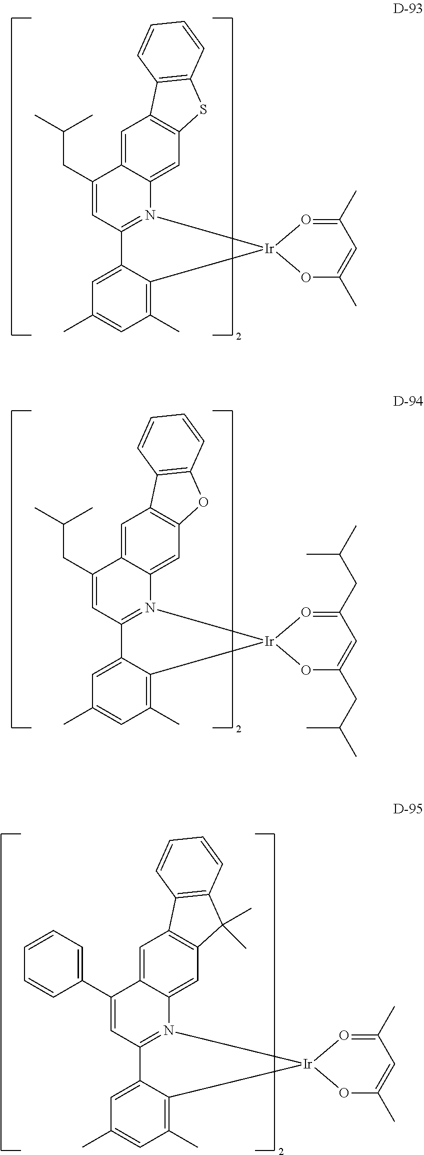

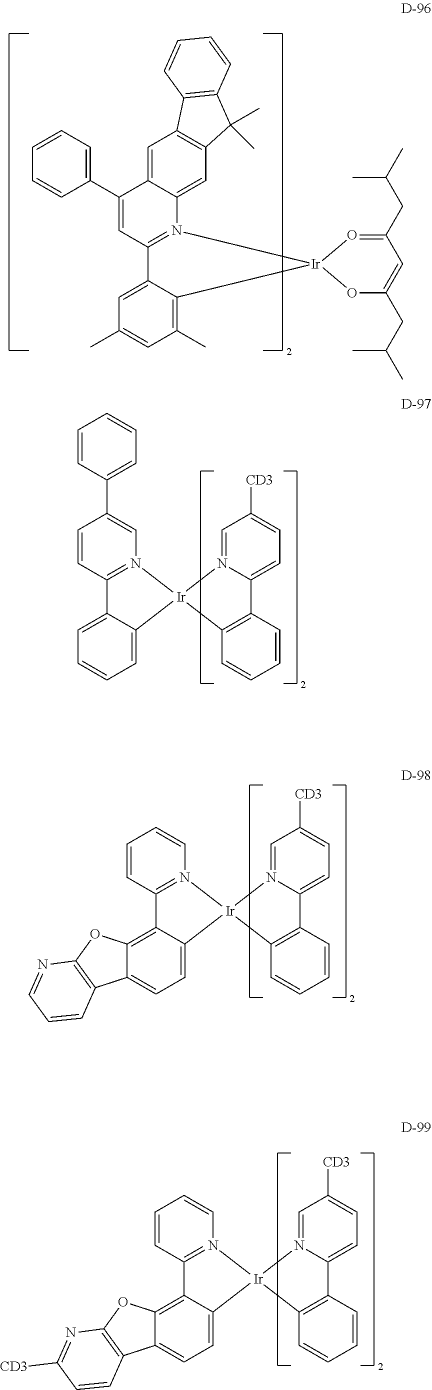

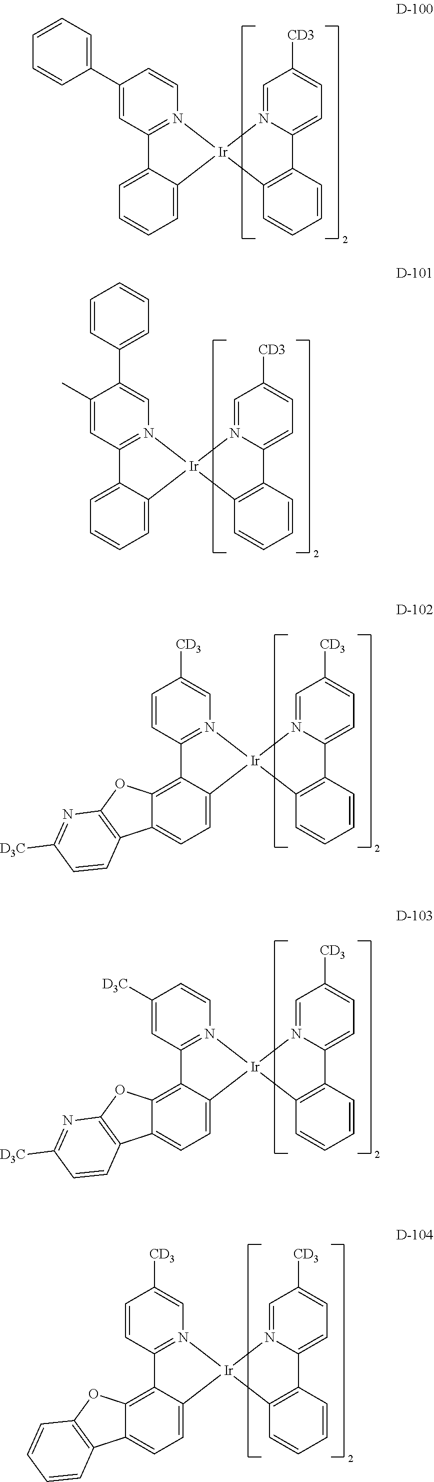

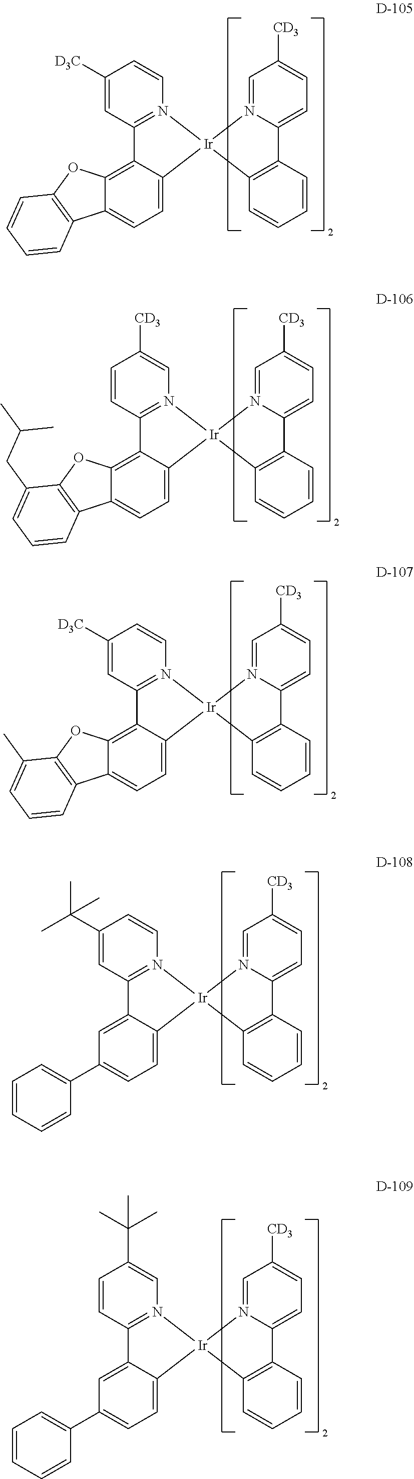

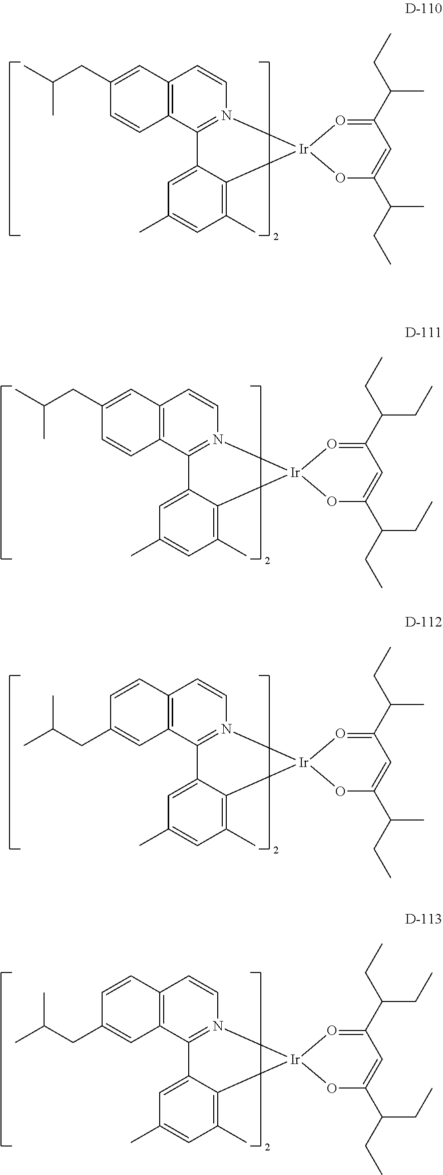

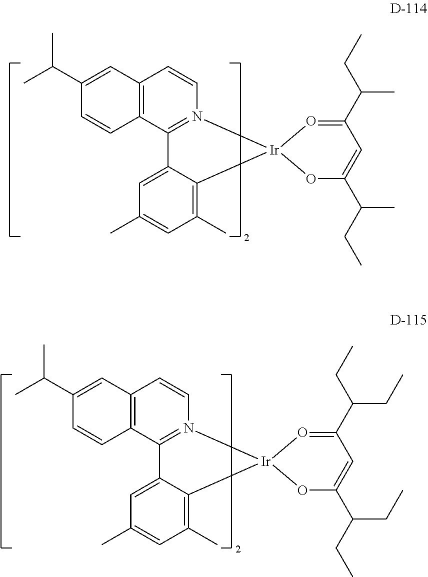

In order to form each layer of the organic electroluminescent device of the present disclosure, dry film-forming methods such as vacuum evaporation, sputtering, plasma, ion plating methods, etc., or wet film-forming methods such as ink jet printing, nozzle printing, slot coating, spin coating, dip coating, flow coating methods, etc., can be used.

When using a wet film-forming method, a thin film can be formed by dissolving or diffusing materials forming each layer into any suitable solvent such as ethanol, chloroform, tetrahydrofuran, dioxane, etc. The solvent can be any solvent where the materials forming each layer can be dissolved or diffused, and where there are no problems in film-formation capability.

The compounds represented by formulas 1 and 2 of the present disclosure may be film-formed by the above-listed methods, commonly by a co-evaporation process or a mixture-evaporation process. The co-evaporation is a mixed deposition method in which two or more materials are placed in a respective individual crucible source and a current is applied to both cells at the same time to evaporate the materials. The mixture-evaporation is a mixed deposition method in which two or more materials are mixed in one crucible source before evaporating them, and a current is applied to the cell to evaporate the materials. Further, if the first and the second host compounds are present in the same layer or different layers in an organic electroluminescent device, the two host compounds may individually form films. For example, the second host compound may be deposited after depositing the first host compound.

In addition, the organic electroluminescent material according to one embodiment may be used as light-emitting materials for a white organic light-emitting device. Various structures have been proposed for the white organic electroluminescent device, for example, a side-by-side structure or a stacking structure depending on the arrangement of R (red), G (green) or YG (yellow green), and B (blue) light emitting parts, or a color conversion material (CCM) method, etc. The present disclosure may also be applied to such white organic electroluminescent device. The organic electroluminescent materials according to the present disclosure may also be applied to the organic electroluminescent device comprising a QD (quantum dot).

The present disclosure may provide a display system comprising the plurality of host materials according to the present disclosure. In addition, it is possible to manufacture a display system or a lighting system by using the organic electroluminescent device of the present disclosure. Specifically, it is possible to produce a display system, e.g., a display system for smart phones, tablets, notebooks, PCs, TVs, or cars; or a lighting system, e.g., an outdoor or indoor lighting system, by using the organic electroluminescent device of the present disclosure.

Hereinafter, the preparation method of the compounds according to the present disclosure and the properties thereof will be explained in detail with reference to the representative compounds of the present disclosure. However, the present disclosure is not limited by the following examples.

### Example 1: Preparation of Compound H2-29

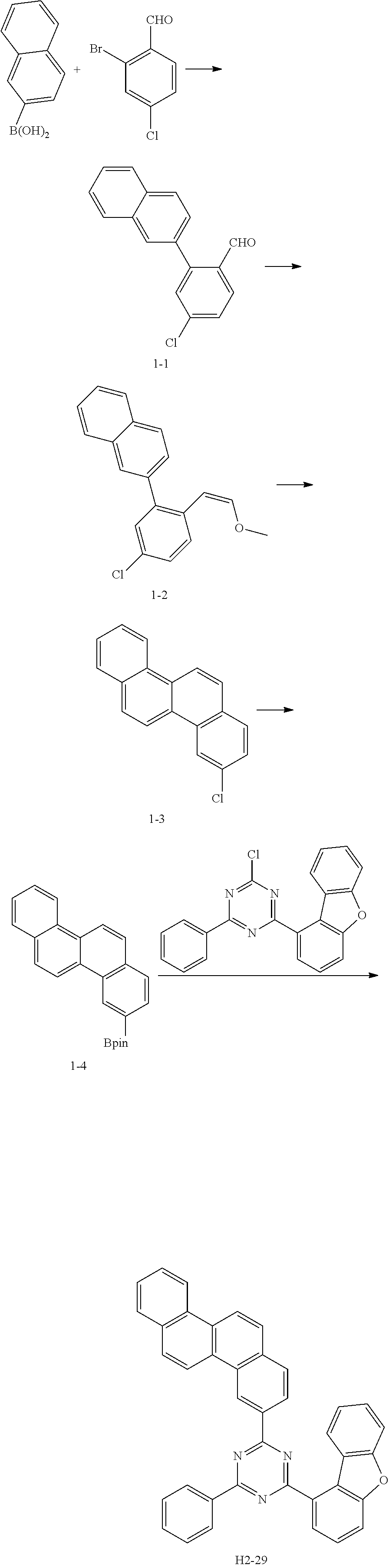

1) Synthesis of Compound 1-1

In a reaction vessel, 50 g of naphthalen-2-yl boronic acid (291 mmol), 63 g of 2-bromo-4-chlorobenzaldehyde (291 mmol), 16.8 g of tetrakis(triphenylphosphine)palladium (14.5 mmol), 77 g of sodium carbonate (727 mmol), 1080 mL of toluene, 240 mL of ethanol, and 360 mL of distilled water were added, and the mixture was stirred at 140° C. for 5 hours. After completion of the reaction, the precipitated solid was washed with distilled water and methanol. The residue was purified by column chromatography to obtain 71 g of compound 1-1 (yield: 92%).

2) Synthesis of Compound 1-2

In a reaction vessel, 71 g of compound 1-1 (268 mmol), 110 g of (methoxymethyl)triphenylphosphonium chloride (321 mmol), and 1300 mL of tetrahydrofuran were added, and the mixture was stirred for 10 minutes. 300 mL of potassium tert-butoxide (1M in THF) was slowly added dropwise to the mixture under 0° C. conditions. The temperature was slowly raised to room temperature and the reaction solution was stirred for 3 hours. Distilled water was added to the reaction solution to complete the reaction, and an organic layer was extracted with ethyl acetate. The extracted organic layer was dried with magnesium sulfate and the solvent was removed by a rotary evaporator. The residue was purified by column chromatography to obtain 71 g of compound 1-2 (yield: 90%).

3) Synthesis of Compound 1-3

In a reaction vessel, 70 g of compound 1-2 (238 mmol), 7 mL of Eaton's reagent, and 1180 mL of chlorobenzene were added, and the mixture was refluxed for 1 hour. After completion of the reaction, the mixture was cooled to room temperature, and an organic layer was extracted with methylene chloride (MC). The extracted organic layer was dried with magnesium sulfate and the solvent was removed by a rotary evaporator. The residue was purified by column chromatography to obtain 60 g of compound 1-3 (yield: 96%).

4) Synthesis of Compound 1-4

In a reaction vessel, 35 g of compound 1-3 (133.2 mmol), 44 g of bis(pinacolato)diboron (173 mmol), 6.1 g of tris(dibenzylideneacetone)dipalladium (6.66 mmol), 5.5 g of 2-dicyclohexylphosphino-2′,6′-dimethoxybiphenyl (s-phos) (13.3 mmol), 39.2 g of potassium acetate (400 mmol), and 666 mL of 1,4-dioxane were added, and the mixture was stirred at 150° C. for 3 hours. After completion of the reaction, the mixture was cooled to room temperature, and an organic layer was extracted with ethyl acetate. The extracted organic layer was dried with magnesium sulfate and the solvent was removed by a rotary evaporator. The residue was purified by column chromatography to obtain 38 g of compound 1-4 (yield: 81%).

5) Synthesis of Compound H2-29

In a reaction vessel, 5 g of compound 1-4 (14.1 mmol), 6.6 g of 2-chloro-4-(dibenzo[b,d]furan-1-yl)-6-phenyl-1,3,5-triazine (18.3 mmol), 0.8 g of tetrakis(triphenylphosphine)palladium (0.7 mmol), 3.9 g of potassium carbonate (28.2 mmol), 42 mL of toluene, 10 mL of ethanol, and 14 mL of distilled water were added, and the mixture was stirred at 140° C. for 8 hours. After completion of the reaction, the mixture was added dropwise to methanol, and the resulting solid was filtered. The resulting solid was purified by column chromatography to obtain 6.8 g of compound H2-29 (yield: 88%).

| | MW | M.P. |
|---|---|---|
| H2-29 | 549.6 | 267° C. |

### Example 2: Preparation of Compound H2-220

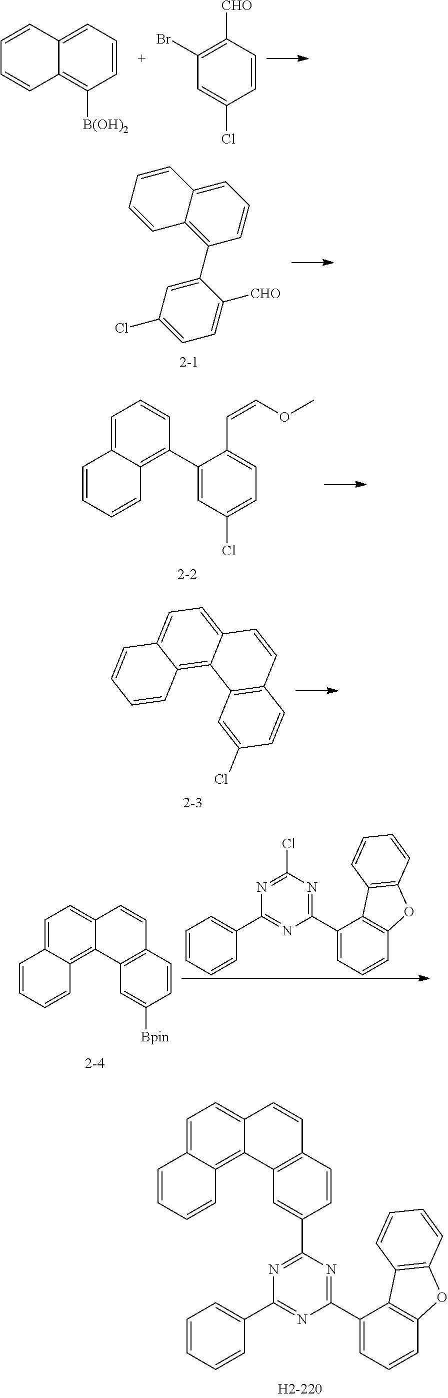

1) Synthesis of Compound 2-1

In a reaction vessel, 40 g of naphthalen-1-yl boronic acid (232 mmol), 51 g of 2-bromo-4-chlorobenzaldehyde (232 mmol), 13.4 g of tetrakis(triphenylphosphine)palladium (11.6 mmol), 62 g of sodium carbonate (582 mmol), 900 mL of toluene, 200 mL of ethanol, and 300 mL of distilled water were added, and the mixture was stirred at 140° C. for 5 hours. After completion of the reaction, the precipitated solid was washed with distilled water and methanol. The residue was purified by column chromatography to obtain 50 g of compound 2-1 (yield: 80%).

2) Synthesis of Compound 2-2

In a reaction vessel, 50 g of compound 2-1 (187.5 mmol), 83 g of (methoxymethyl)triphenylphosphonium chloride (243.7 mmol), and 935 mL of tetrahydrofuran were added, and the mixture was stirred for 10 minutes. 250 mL of potassium tert-butoxide (1M in THF) was slowly added dropwise to the mixture under 0° C. conditions. The temperature was slowly raised to room temperature and the reaction solution was stirred for 3 hours. Distilled water was added to the reaction solution to complete the reaction, and an organic layer was extracted with ethyl acetate. The extracted organic layer was dried with magnesium sulfate and the solvent was removed by a rotary evaporator. The residue was purified by column chromatography to obtain 52 g of compound 2-2 (yield: 95%).

3) Synthesis of Compound 2-3

In a reaction vessel, 62 g of compound 2-2 (210 mmol), 21 mL of Eaton's reagent, and 1000 mL of chlorobenzene were added, and the mixture was refluxed for 2 hours. After completion of the reaction, the mixture was cooled to room temperature, and an organic layer was extracted with methylene chloride (MC). The extracted organic layer was dried with magnesium sulfate and the solvent was removed by a rotary evaporator. The residue was purified by column chromatography to obtain 12.5 g of compound 2-3 (yield: 23%).

4) Synthesis of Compound 2-4

In a reaction vessel, 12.5 g of compound 2-3 (47.6 mmol), 15.7 g of bis(pinacolato)diboron (61.9 mmol), 2.2 g of tris(dibenzylideneacetone)dipalladium (2.38 mmol), 1.96 g of 2-dicyclohexylphosphino-2′,6′-dimethoxybiphenyl (s-phos) (4.76 mmol), 14 g of potassium acetate (143 mmol), and 240 mL of 1,4-dioxane were added, and the mixture was stirred at 150° C. for 3 hours. After completion of the reaction, the mixture was cooled to room temperature, and an organic layer was extracted with ethyl acetate. The extracted organic layer was dried with magnesium sulfate and the solvent was removed by a rotary evaporator. The residue was purified by column chromatography to obtain 11.1 g of compound 2-4 (yield: 66%).

5) Synthesis of Compound H2-220

In a reaction vessel, 4 g of compound 2-4 (11.3 mmol), 5.3 g of 2-chloro-4-(dibenzo[b,d]furan-1-yl)-6-phenyl-1,3,5-triazine (14.7 mmol), 0.65 g of tetrakis(triphenylphosphine)palladium (0.6 mmol), 3.1 g of potassium carbonate (22.6 mmol), 33 mL of toluene, 5 mL of ethanol, and 11 mL of distilled water were added, and the mixture was stirred at 140° C. for 7 hours. After completion of the reaction, the mixture was added dropwise to methanol, and the resulting solid was filtered. The resulting solid was purified by column chromatography to obtain 4.1 g of compound H2-220 (yield: 66%).

| | MW | M.P. |
|---|---|---|
| H2-220 | 549.6 | 208° C. |

### Example 3: Preparation of Compound H1-85

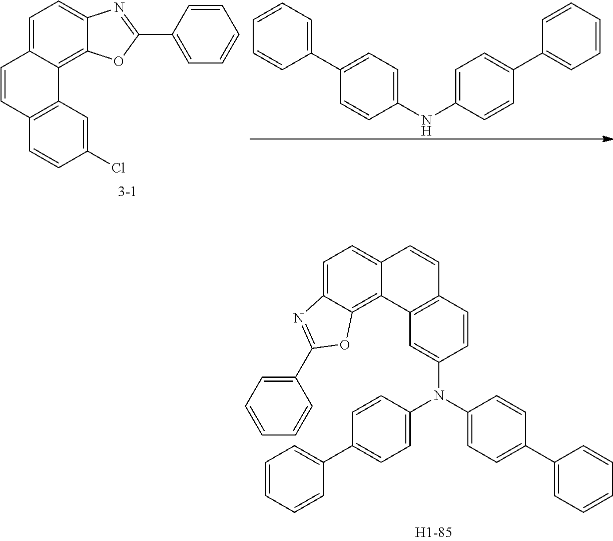

In a reaction vessel, 5.0 g of compound 3-1 (15.2 mmol), 4.9 g of di([1,1′-biphenyl]-4-yl)amine (15.2 mmol), 0.2 g of Pd(OAc)₂ (0.8 mmol), 0.8 mL of P(tert-Bu)₃ (1.5 mmol), 2.9 g of sodium tert-butoxide (30.4 mmol), and 76 mL of xylene were added, and the mixture was stirred at 160° C. for 5 hours. After completion of the reaction, the precipitated solid was washed with distilled water and methanol. The residue was purified by column chromatography to obtain 5.5 g of compound H1-85 (yield: 59%).

### Example 4: Preparation of Compound H1-51

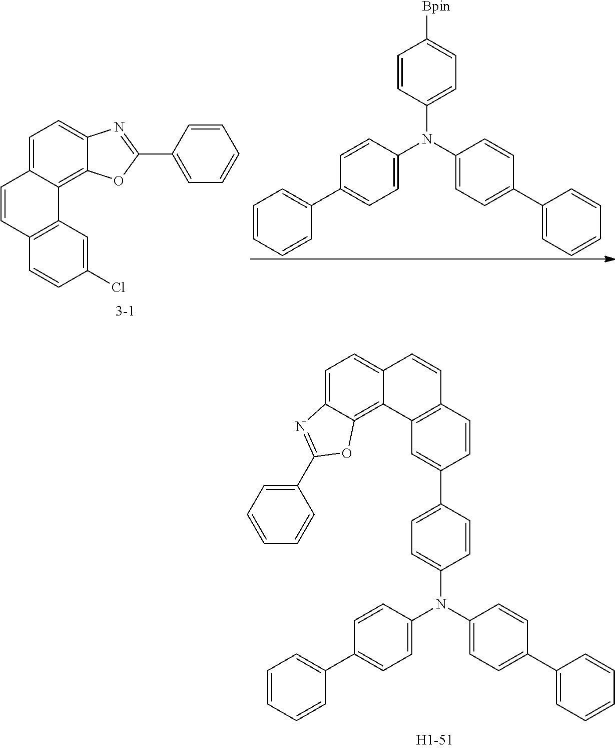

In a reaction vessel, 4 g of compound 3-1 (12 mmol), 6.8 g of bis(biphenyl-4-yl)[4-(4,4,5,5-tetramethyl-[1,3,2]-dioxaborolan-2-yl)phenyl]amine (13 mmol), 0.3 g of palladium(II) acetate (Pd(OAC)₂) (1 mmol), 0.9 g of 2-dicyclohexylphosphino-2′,6′-dimethoxybiphenyl (s-Phos) (2 mmol), 11.5 g of cesium carbonate (Cs₂CO₃) (35 mmol), 60 mL of o-xylene, 15 mL of ethanol (EtOH), and 15 mL of distilled water were added, and the mixture was stirred under reflux at 150° C. for 3 hours. After completion of the reaction, the mixture was washed with distilled water, and an organic layer was extracted with ethyl acetate. The extracted organic layer was dried with magnesium sulfate and the solvent was removed by a rotary evaporator. The residue was purified by column chromatography to obtain 2.2 g of compound H1-51 (yield: 27%).

### Example 5: Preparation of Compound H1-80

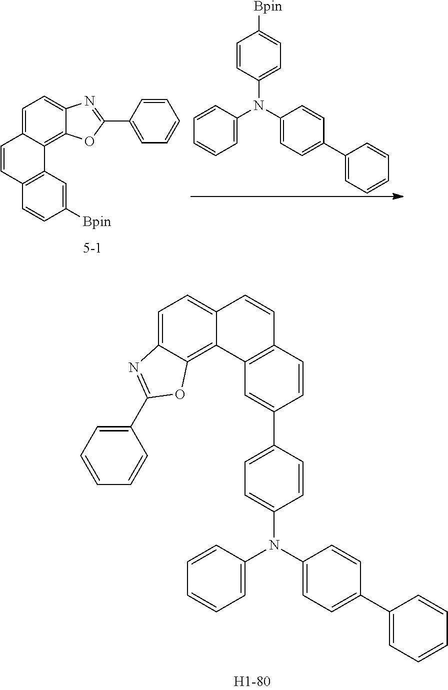

In a reaction vessel, 4.8 g of compound 6-1 (11.34 mmol), 5 g of N-(4-bromophenyl)-N-phenyl-[1,1′-biphenyl]-4-amine (12.47 mmol), 0.4 g of tetrakis(triphenylphosphine)palladium (Pd(PPh₃)₄) (0.34 mmol), 3.0 g of sodium carbonate (Na₂CO₃) (28.35 mmol), 57 mL of toluene, 14 mL of ethanol, and 14 mL of distilled water were added, and the mixture was stirred at 120° C. for 4 hours. After completion of the reaction, the mixture was added dropwise to methanol, and the resulting solid was filtered. The resulting solid was purified by column chromatography to obtain 1.4 g of compound H1-80 (yield: 20.0%).

### Example 6: Preparation of Compound H1-46

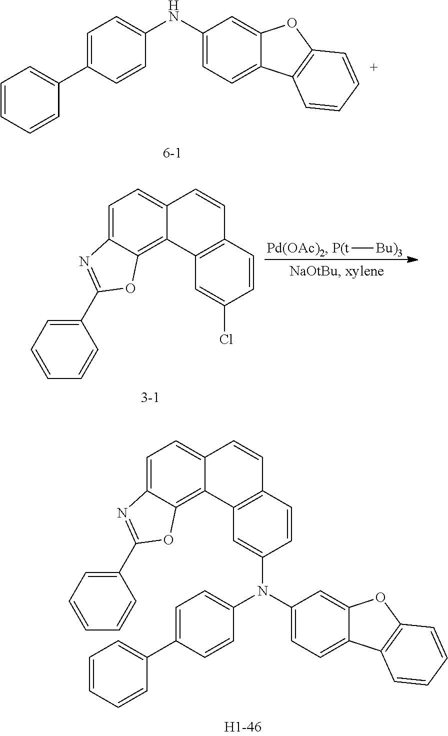

In a flask, 25 g of compound 6-1 (74.48 mmol), 42.58 g of compound 3-1 (81.93 mmol), 0.16 g of Pd(OAc)₂ (7.5 mmol), 0.28 g of P(t-Bu)₃ (7.5 mmol), 14.31 g of NaOt-Bu (150 mmol), and 284.09 mL of o-xylene were added, and the mixture was stirred at 160° C. for 2 hours. After completion of the reaction, the mixture was cooled to room temperature, and an organic layer was extracted with ethyl acetate. The residual moisture was removed with magnesium sulfate. The residue was dried and separated by column chromatography to obtain 23.4 g of compound H1-46 (yield: 50%).

| | MW | M.P. |
|---|---|---|
| H1-46 | 628.22 | 256.5° C. |

### Example 7: Preparation of Compound H1-43

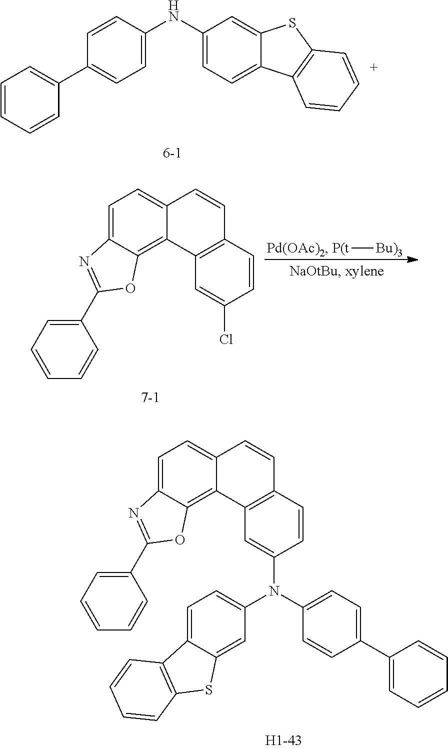

In a flask, 20 g of compound 7-1 (56.96 mmol), 18.8 g of compound 3-1 (57.13 mmol), 0.13 g of Pd(OAc)₂ (5.7 mmol), 0.22 g of P(t-Bu)₃ (5.7 mmol), 11 g of NaOt-Bu (113.92 mmol), and 227.27 mL of o-xylene were added, and the mixture was stirred at 160° C. for 2 hours. After completion of the reaction, the mixture was cooled to room temperature, and an organic layer was extracted with ethyl acetate. The residual moisture was removed with magnesium sulfate. The residue was dried and separated by column chromatography to obtain 12.5 g of compound H1-43 (yield: 34%).

| | MW | M.P. |
|---|---|---|
| H1-43 | 644.19 | 249° C. |

### Example 8: Preparation of Compound H1-27

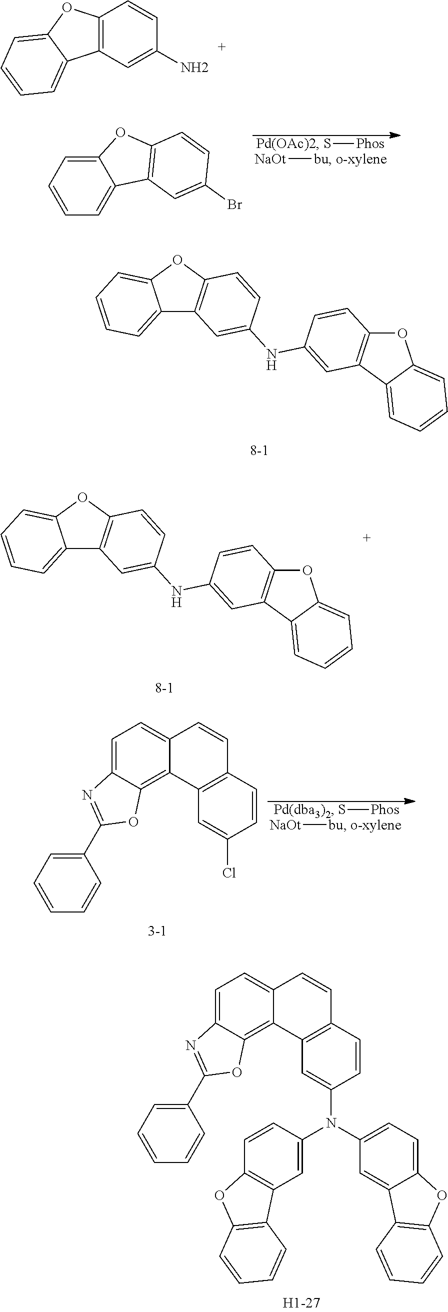

1) Synthesis of Compound 8-1

In a flask, 20 g of dibenzofuran-2-amine (144.7 mmol), 23.8 g of 2-bromo dibenzofuran (96.47 mmol), 1.1 g of Pd(OAc)₂ (4.82 mmol), 3.9 g of S-Phos (9.65 mmol), 13.9 g of NaOt-Bu (144.7 mmol), and 485 mL of o-xylene were added, and the mixture was stirred at 160° C. for 3 hours. After completion of the reaction, the mixture was cooled to room temperature, and an organic layer was extracted with ethyl acetate. The residual moisture was removed with magnesium sulfate. The residue was dried and separated by column chromatography to obtain 4.9 g of compound 8-1 (yield: 10%).

2) Synthesis of Compound H1-27

In a flask, 4.9 g of compound 8-1 (12.76 mmol), 4.2 g of compound 3-1 (14.0 mmol), 0.584 g of Pd(dba₃)₂ (0.638 mmol), 0.523 g of S-Phos (1.276 mmol), 1.8 g of NaOt-Bu (19.14 mmol), and 65 mL of o-xylene were added, and the mixture was stirred at 160° C. for 2 hours. After completion of the reaction, the mixture was cooled to room temperature, and an organic layer was extracted with ethyl acetate. The residual moisture was removed with magnesium sulfate. The residue was dried and separated by column chromatography to obtain 5.6 g of compound H1-27 (yield: 68.3%).

| | MW | M.P. |
|---|---|---|
| H1-27 | 642.19 | 237° C. |

### Example 9: Preparation of Compound H2-216

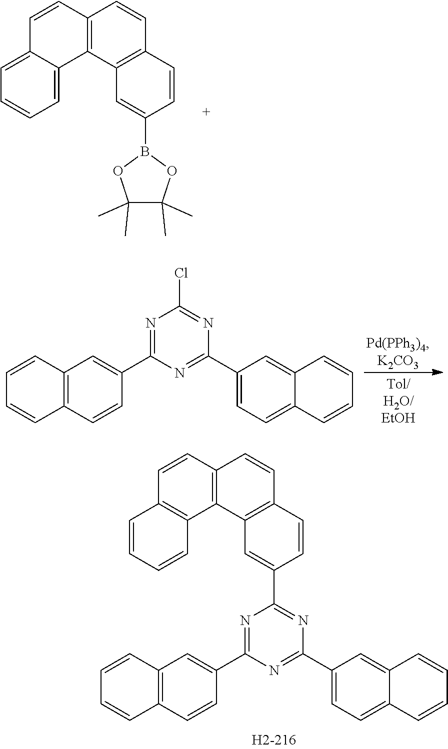

In a flask, 3.5 g of 2-benzo[c]phenanthren-2-yl-4,4,5,5-tetramethyl(1,3,2-dioxaborolane) (9.8 mmol), 4.7 g of 2-chloro-2,4-dinaphthalenyl-1,3,5-triazine (12.8 mmol), 0.56 g of Pd(PPh₃)₄ (0.49 mmol), and 3.4 g of K₂CO₃ (24.5 mmol) were dissolved in 50 mL of toluene, 25 mL of ethanol, and 25 mL of water, and the mixture was refluxed at 130° C. for 24 hours. After completion of the reaction, an organic layer was extracted with ethyl acetate. The residual moisture was removed with magnesium sulfate. The residue was dried and separated by column chromatography to obtain 2.5 g of compound H2-216 (yield: 49.6%).

| | MW | M.P. |
|---|---|---|
| H2-216 | 559.20 | 242.4° C. |

### Example 10: Preparation of Compound H2-246

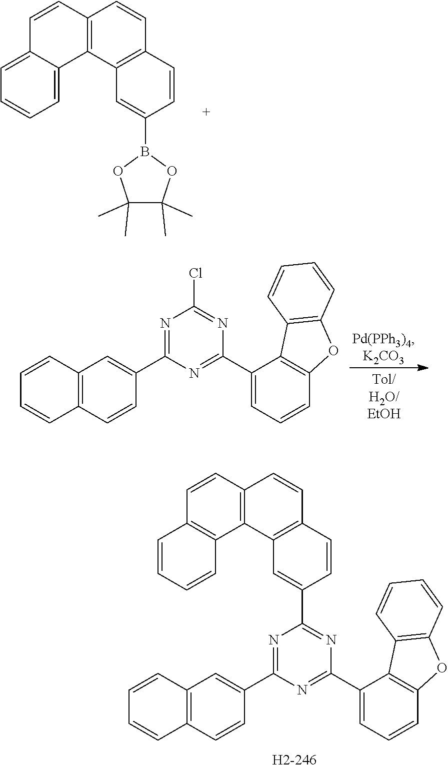

In a flask, 3.5 g of 2-benzo[c]phenanthren-2-yl-4,4,5,5-tetramethyl(1,3,2-dioxaborolane) (9.8 mmol), 3.3 g of 2-chloro-4-dibenzo[d,b]furan-1-yl-6-(naphthalen-2-yl)-1,3,5-triazine (8.2 mmol), 0.47 g of Pd(PPh₃)₄ (0.47 mmol), and 2.8 g of K₂CO₃ (20.5 mmol) were dissolved in 50 mL of toluene, 25 mL of ethanol, and 25 mL of water, and the mixture was refluxed at 130 for 4 hours. After completion of the reaction, an organic layer was extracted with ethyl acetate. The residual moisture was removed with magnesium sulfate. The residue was dried and separated by column chromatography to obtain 2.5 g of compound H2-246 (yield: 50.8%).

| | MW | M.P. |
|---|---|---|
| H2-246 | 559.30 | 274.9° C. |

### Example 11: Preparation of Compound H2-32

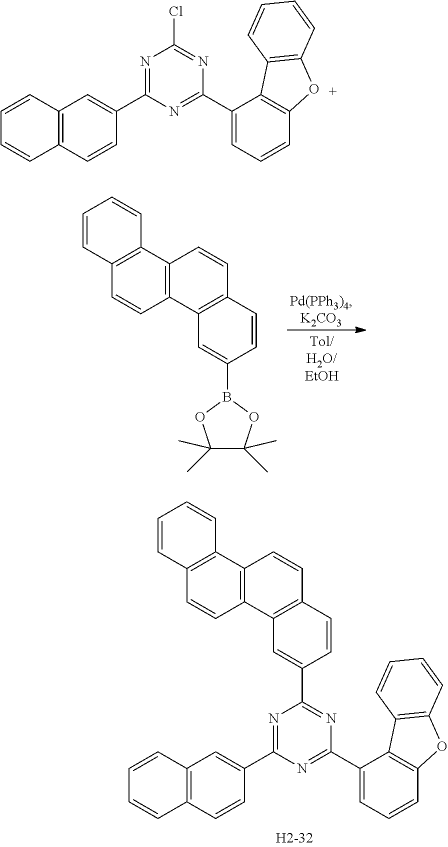

In a flask, 3.8 g of 2-chloro-4-(dibenzo[b,d]furan-1-yl)-6-(naphthalen-2-yl)-1,3,5-triazine (9.4 mmol), 4 g of 2-chrysen-3-yl-4,4,5,5-tetramethyl-(1,3,2-dioxaborolane) (11.3 mmol), 0.54 g of Pd(PPh₃)₄ (0.47 mmol), and 3.3 g of K₂CO₃ (23.5 mmol) were dissolved in 50 mL of toluene, 25 mL of ethanol, and 25 mL of water, and the mixture was refluxed at 130° C. for 4 hours. After completion of the reaction, an organic layer was extracted with ethyl acetate. The residual moisture was removed with magnesium sulfate. The residue was dried and separated by column chromatography to obtain 5 g of compound H2-32 (yield: 94%).

| | MW | M.P. |
|---|---|---|
| H2-32 | 559.30 | 292.8° C. |

### Example 12: Preparation of Compound H1-126

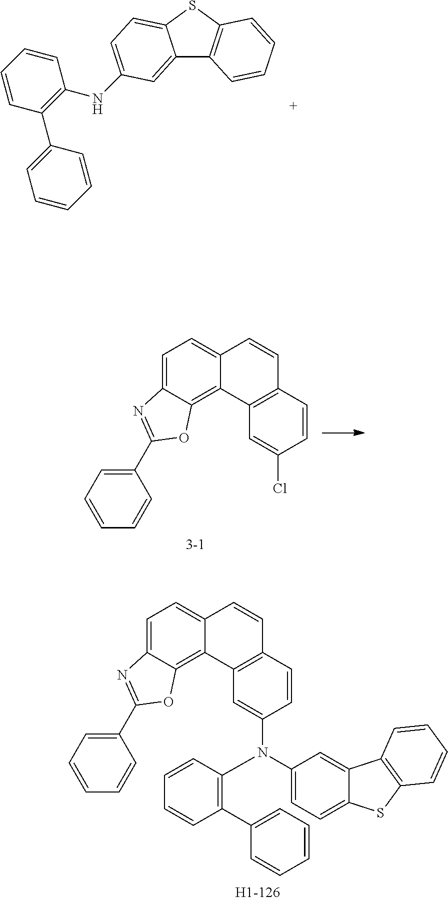 [SMILES: c1ccc(-c2ccccc2Nc2ccc3sc4ccccc4c3c2)cc1 ; Clc1ccc2ccc3ccc4nc(-c5ccccc5)oc4c3c2c1 ; c1ccc(-c2nc3ccc4ccc5ccc(N(c6ccc7sc8ccccc8c7c6)c6ccccc6-c6ccccc6)cc5c4c3o2)cc1]

In a flask, 10 g of N-([1,1′-biphenyl]-2-yl)dibenzo[b,d]thiophene-2-amine (28.45 mmol), 8.5 g of compound 3-1 (25.9 mmol), 1.2 g of Pd₂(dba)₃ (1.295 mmol), 1 g of S-Phos (2.59 mmol), 6.2 g of NaOt-Bu (64.75 mmol), and 130 mL of o-xylene were added, and the mixture was stirred at 160° C. for 2 hours. After completion of the reaction, the mixture was cooled to room temperature, and an organic layer was extracted with ethyl acetate. The residual moisture was removed with magnesium sulfate. The residue was dried and separated by column chromatography to obtain 8.7 g of compound H1-126 (yield: 52%).

| | MW | M.P. |
|---|---|---|
| H1-126 | 644.19 | 256.2° C. |

### Example 13: Preparation of Compound H1-113

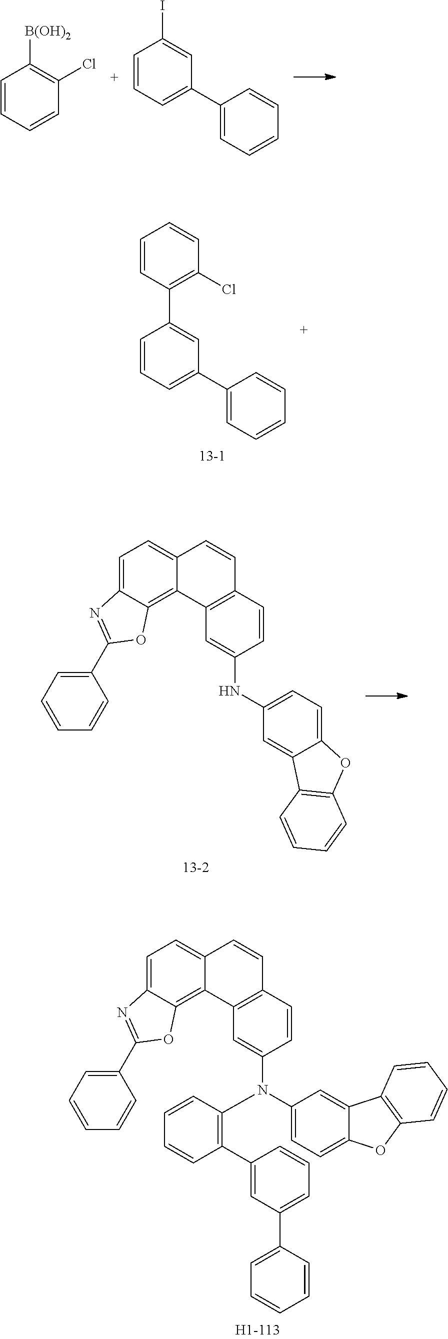 [SMILES: OB(O)c1ccccc1Cl ; Ic1cccc(-c2ccccc2)c1 ; Clc1ccccc1-c1cccc(-c2ccccc2)c1 ; c1ccc(-c2nc3ccc4ccc5ccc(Nc6ccc7oc8ccccc8c7c6)cc5c4c3o2)cc1 ; c1ccc(-c2cccc(-c3ccccc3N(c3ccc4oc5ccccc5c4c3)c3ccc4ccc5ccc6nc(-c7ccccc7)oc6c5c4c3)c2)cc1]

1) Synthesis of Compound 13-1

In a flask, 30 g of (2-chlorophenyl)boronic acid (75.2 mmol), 21 g of 3-iodo-1,1′-biphenyl (75.2 mmol), 4.3 g of Pd(PPh₃)₄ (3.78 mmol), 26 g of K₂CO₃ (188 mmol), 230 mL of toluene, 110 mL of EtOH, and 110 mL of H₂O were added, and the mixture was stirred at 130° C. for 1 hour. After completion of the reaction, the mixture was cooled to room temperature, and an organic layer was extracted with ethyl acetate. The residual moisture was removed with magnesium sulfate. The residue was dried and separated by column chromatography to obtain 19 g of compound 13-1 (yield: 95.7%).

2) Synthesis of compound H1-113

In a flask, 4.65 g of compound 13-1 (17.64 mmol), 7 g of compound 13-2 (14.7 mmol), 0.67 g of Pd₂(dba)₃ (0.735 mmol), 0.6 g of S-Phos (1.47 mmol), 2.1 g of NaOt-Bu (22.05 mmol), and 70 mL of o-xylene were added, and the mixture was stirred at 160° C. for 2 hours. After completion of the reaction, the mixture was cooled to room temperature, and an organic layer was extracted with ethyl acetate. The residual moisture was removed with magnesium sulfate. The residue was dried and separated by column chromatography to obtain 4.1 g of compound H1-113 (yield: 39%).

| | MW | M.P. |
|---|---|---|
| H1-113 | 704.8 | 196.3° C. |

### Example 14: Preparation of Compound H-110

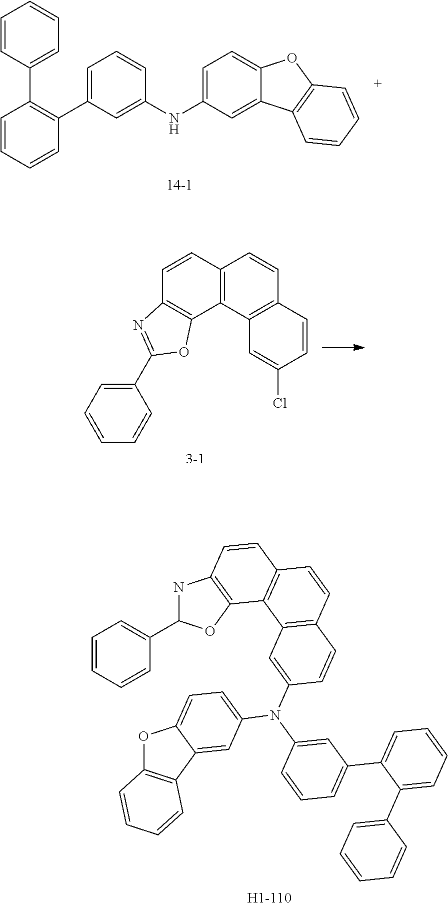 [SMILES: c1ccc(-c2ccccc2-c2cccc(Nc3ccc4oc5ccccc5c4c3)c2)cc1 ; Clc1ccc2ccc3ccc4nc(-c5ccccc5)oc4c3c2c1 ; c1ccc(-c2ccccc2-c2cccc(N(c3ccc4oc5ccccc5c4c3)c3ccc4ccc5ccc6c(c5c4c3)OC(c3ccccc3)N6)c2)cc1]

In a flask, 91.7 g of compound 14-1 (222.8 mmol), 70.0 g of compound 3-1 (212.2 mmol), 0.56 g of Pd(OAc)₂ (2.5 mmol), 1.01 g of Xphos (2.11 mmol), and 30.6 g of NaOtBu (318.4 mmol) were dissolved in 2500 mL of toluene, and the mixture was stirred under reflux for 48 hours. After completion of the reaction, an organic layer was extracted with EA/H₂O, and separated by column chromatography to obtain 44.9 g of compound H1-110 (yield: 30%).

| | MW | M.P. |
|---|---|---|
| H1-110 | 704.8 | 200° C. |

### Example 15: Preparation of Compound H1-102

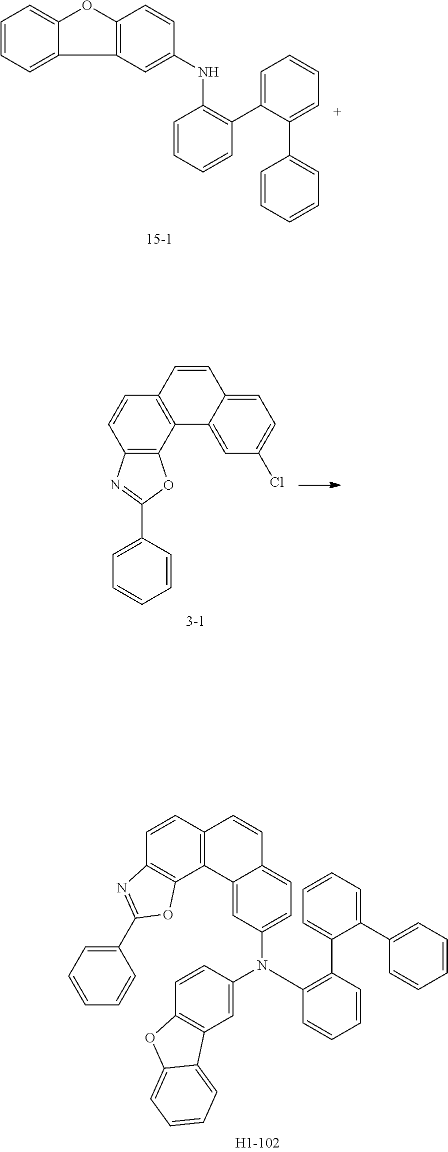 [SMILES: c1ccc(-c2ccccc2-c2ccccc2Nc2ccc3oc4ccccc4c3c2)cc1 ; Clc1ccc2ccc3ccc4nc(-c5ccccc5)oc4c3c2c1 ; c1ccc(-c2nc3ccc4ccc5ccc(N(c6ccc7oc8ccccc8c7c6)c6ccccc6-c6ccccc6-c6ccccc6)cc5c4c3o2)cc1]

In a flask, 58 g of compound 15-1 (140.9 mmol), 18.2 g of compound 3-1 (55.2 mmol), 0.25 g of Pd(OAc)₂ (1.11 mmol), 1.05 g of Xphos (2.2 mmol), and 10.6 g of NaOtBu (110.3 mmol) were dissolved in 1000 mL of toluene, and the mixture was stirred under reflux for 48 hours. After completion of the reaction, an organic layer was extracted with EA/H₂O, and separated by column chromatography to obtain 17.9 g of compound H1-102 (yield: 46%).

| | MW | M.P. |
|---|---|---|
| H1-102 | 704.8 | 256° C. |

### Example 16: Preparation of Compound H1-47

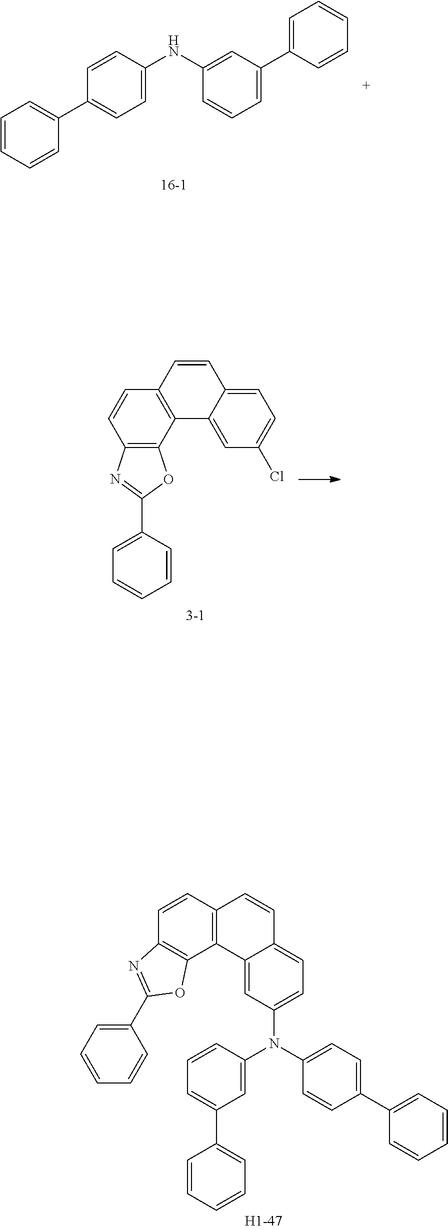 [SMILES: c1ccc(-c2ccc(Nc3cccc(-c4ccccc4)c3)cc2)cc1 ; Clc1ccc2ccc3ccc4nc(-c5ccccc5)oc4c3c2c1 ; c1ccc(-c2ccc(N(c3cccc(-c4ccccc4)c3)c3ccc4ccc5ccc6nc(-c7ccccc7)oc6c5c4c3)cc2)cc1]

In a flask, 31 g of compound 16-1 (96.44 mmol), 31.8 g of compound 3-1 (96.42 mmol), 0.11 g of Pd(OAc)₂ (0.48 mmol), 0.46 g of Xphos (0.96 mmol), and 13.91 g of NaOtBu (144.7 mmol) were dissolved in 900 mL of toluene, and the mixture was stirred under reflux for 24 hours. After completion of the reaction, an organic layer was extracted with EA/H2, and separated by column chromatography to obtain 17.8 g of compound H1-47 (yield: 30%).

| | MW | M.P. |
|---|---|---|
| H1-47 | 614.7 | 137.6° C. |

### Example 17: Preparation of Compound H1-127

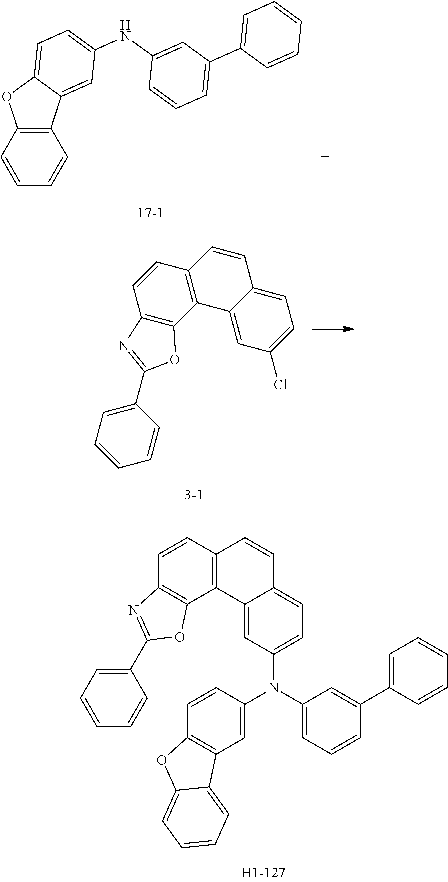 [SMILES: c1ccc(-c2cccc(Nc3ccc4oc5ccccc5c4c3)c2)cc1 ; Clc1ccc2ccc3ccc4nc(-c5ccccc5)oc4c3c2c1 ; c1ccc(-c2cccc(N(c3ccc4oc5ccccc5c4c3)c3ccc4ccc5ccc6nc(-c7ccccc7)oc6c5c4c3)c2)cc1]

In a flask, 5.6 g of compound 17-1 (16.67 mmol), 5.0 g of compound 3-1 (15.16 mmol), 0.7 g of Pd₂(dba)₃ (0.76 mmol), 0.6 g of Sphos (1.52 mmol), and 3.6 g of NaOtBu (37.9 mmol) were dissolved in 80 mL of xylene, and the mixture was stirred under reflux for 2 hours. After completion of the reaction, an organic layer was extracted with EA/H₂O, and separated by column chromatography to obtain 2.9 g of compound H1-127 (yield: 30%).

| | MW | M.P. |
|---|---|---|
| H1-127 | 628.7 | 210.9° C. |

### Example 18: Preparation of Compound H1-104

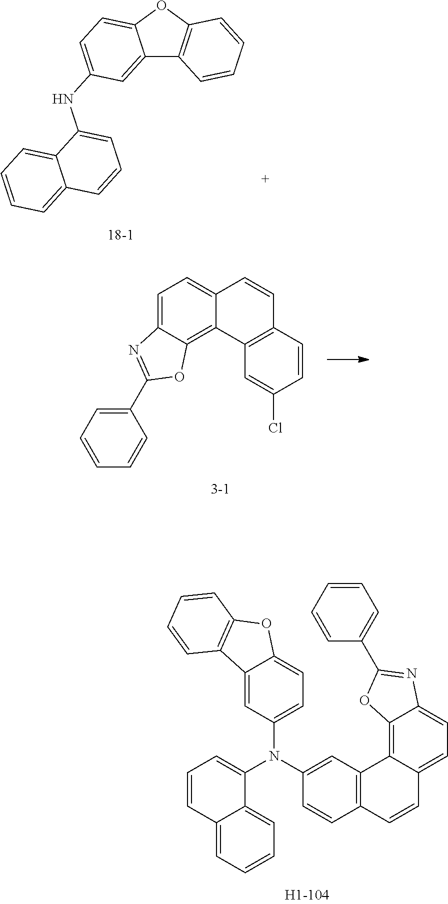 [SMILES: c1ccc2c(Nc3ccc4oc5ccccc5c4c3)cccc2c1 ; Clc1ccc2ccc3ccc4nc(-c5ccccc5)oc4c3c2c1 ; c1ccc(-c2nc3ccc4ccc5ccc(N(c6ccc7oc8ccccc8c7c6)c6cccc7ccccc67)cc5c4c3o2)cc1]

In a flask, 55.0 g of compound 18-1 (177.8 mmol), 56.0 g of compound 3-1 (169.8 mmol), 0.77 g of Pd₂(dba)₃ (0.84 mmol), 0.70 g of Sphos (1.7 mmol), and 3.6 g of NaOtBu (252.9 mmol) were dissolved in 440 mL of xylene, and the mixture was stirred under reflux for 5 hours. After completion of the reaction, an organic layer was extracted with EA/H₂O, and separated by column chromatography to obtain 28.6 g of compound H1-104 (yield: 28%).

| | MW | M.P. |
|---|---|---|
| H1-104 | 602.7 | 257.5° C. |

### Example 19: Preparation of Compound H1-28

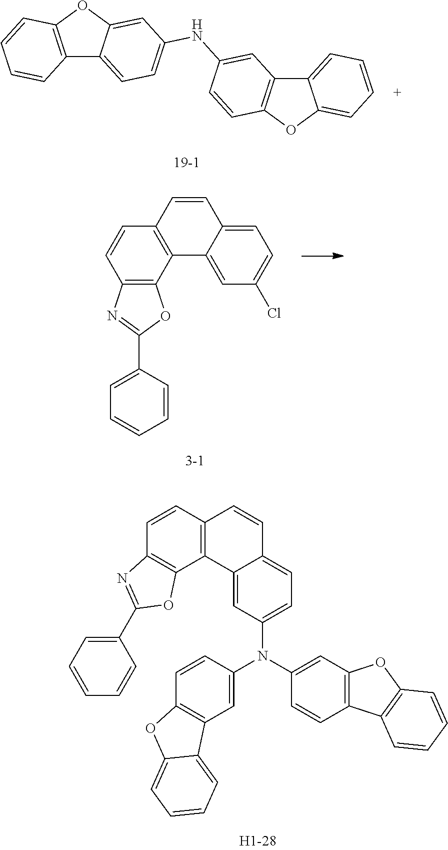 [SMILES: c1ccc2c(c1)oc1cc(Nc3ccc4oc5ccccc5c4c3)ccc12 ; Clc1ccc2ccc3ccc4nc(-c5ccccc5)oc4c3c2c1 ; c1ccc(-c2nc3ccc4ccc5ccc(N(c6ccc7c(c6)oc6ccccc67)c6ccc7oc8ccccc8c7c6)cc5c4c3o2)cc1]

In a flask, 23.3 g of compound 19-1 (66.7 mmol), 20.0 g of compound 3-1 (60.6 mmol), 0.07 g of Pd(OAc)₂ (0.31 mmol), 0.29 g of Xphos (0.61 mmol), and 11.6 g of NaOtBu (120.7 mmol) were dissolved in 300 mL of toluene, and the mixture was stirred under reflux for 6 hours. After completion of the reaction, an organic layer was extracted with EA/H₂O, and separated by column chromatography to obtain 12.4 g of compound H1-28 (yield: 32%).

| | MW | M.P. |
|---|---|---|
| H1-28 | 642.7 | 294.7° C. |

### Example 20: Preparation of Compound H2-179

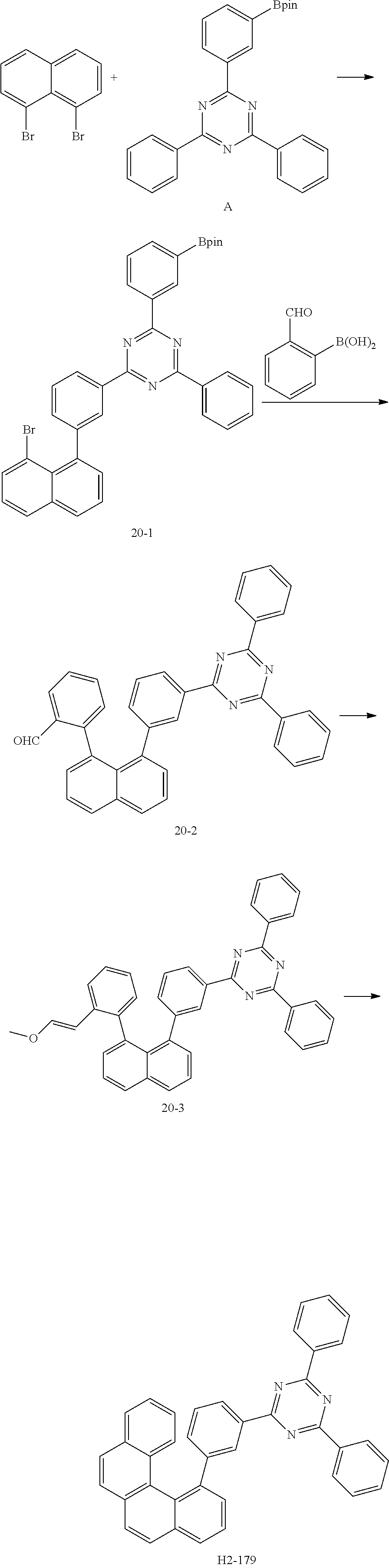

1) Synthesis of Compound 20-1

In a reaction vessel, 15 g of 1,8-dibromonaphthalene (52.5 mmol), 22.8 g of compound A (52.5 mmol), 3.0 g of tetrakis(triphenylphosphine)palladium(0) (2.6 mmol), 18.1 g of potassium carbonate (131.3 mmol), 280 mL of tetrahydrofuran, and 70 mL of distilled water were added, and the mixture was stirred at 110° C. for 9 hours. After completion of the reaction, the mixture was cooled to room temperature, and an organic layer was extracted with ethyl acetate. The extracted organic layer was dried with magnesium sulfate and the solvent was removed by a rotary evaporator. The residue was purified by column chromatography to obtain 8.5 g of compound 20-1 (yield: 31%).

2) Synthesis of Compound 20-2

In a reaction vessel, 8.5 g of compound 20-1 (16.5 mmol), 12.3 g of (2-formylphenyl)boronic acid (82.6 mmol), 1.9 g of tetrakis(triphenylphosphine)palladium(0) (2.6 mmol), 16.1 g of cesium carbonate (49.5 mmol), 100 mL of toluene, 25 mL of ethanol, and 25 mL of distilled water were added, and the mixture was stirred under reflux at 140° C. for 2 hours. After completion of the reaction, the mixture was cooled to room temperature, and an organic layer was extracted with ethyl acetate. The extracted organic layer was dried with magnesium sulfate and the solvent was removed by a rotary evaporator. The residue was purified by column chromatography to obtain 6.2 g of compound 20-2 (yield: 69%).

3) Synthesis of Compound 20-3

In a reaction vessel, 6.2 g of compound 20-2 (11.4 mmol), 4.8 g of (methoxymethyl)triphenylphosphonium chloride (14.0 mmol), and 57 mL of tetrahydrofuran were added, and the mixture was stirred for 10 minutes. 14.2 mL of potassium tert-butoxide (1M in THF) was slowly added dropwise to the mixture under 0° C. conditions. The temperature was slowly raised to room temperature and the reaction solution was stirred for 3 hours. Distilled water was added to the reaction solution to complete the reaction, and an organic layer was extracted with ethyl acetate. The extracted organic layer was dried with magnesium sulfate and the solvent was removed by a rotary evaporator. The residue was purified by column chromatography to obtain 5.9 g of compound 20-3 (yield: 91%).

4) Synthesis of Compound H2-179

In a reaction vessel, 5.4 g of compound 20-3 (9.51 mmol), 2.4 mL of boron trifluoride diethyl etherate (19.1 mmol), and 95 mL of methylene chloride (MC) were added, and the mixture was stirred for 3 hours. After completion of the reaction, an organic layer was extracted with methylene chloride (MC). The extracted organic layer was dried with magnesium sulfate and the solvent was removed by a rotary evaporator. The residue was purified by column chromatography to obtain 2.11 g of compound H2-179 (yield: 41%).

| | MW | M.P. |
|---|---|---|
| H2-179 | 535.6 | 223° C. |

### Example 21: Preparation of Compound H2-89

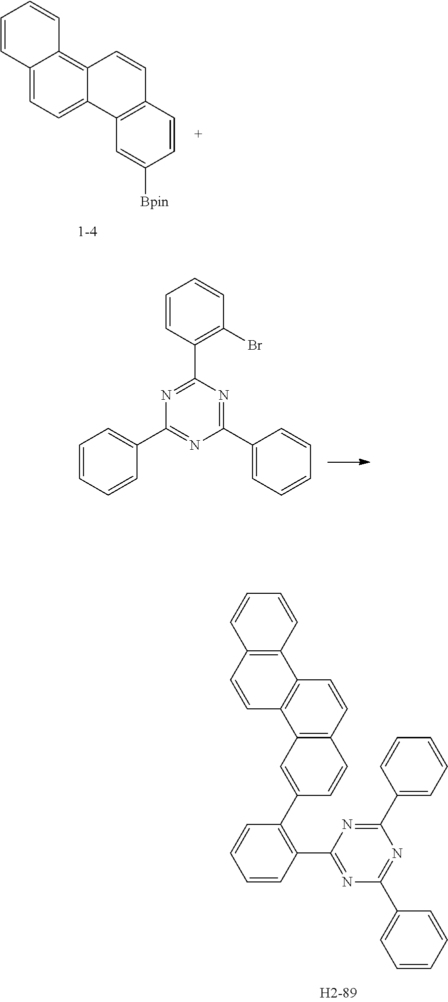

In a reaction vessel, 5.0 g of compound 1-4 (14.11 mmol), 6.0 g of 2-(2-bromophenyl)-4,6-diphenyl-1,3,5-triazine (15.52 mmol), 0.8 g of tetrakis(trphenylphosphine)palladium(0) (0.71 mmol), 5.8 g of potassium carbonate (42.34 mmol), 100 mL of toluene, 25 mL of ethanol, and 25 mL of distilled water were added, and the mixture was stirred at 120° C. for 2 hours. After completion of the reaction, the precipitated solid was washed with distilled water and methanol. The residue was purified by column chromatography to obtain 2.8 g of compound H2-89 (yield: 37%).

| | MW | M.P. |
|---|---|---|
| H2-89 | 535.6 | 208° C. |

### Example 22: Preparation of Compound H2-250

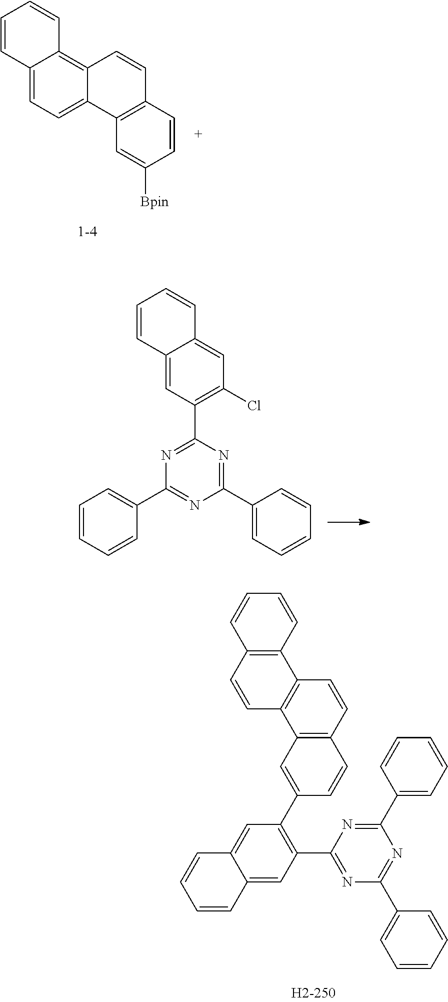

In a reaction vessel, 5.0 g of compound 1-4 (14.11 mmol), 5.5 g of 2-(3-chloronaphthalen-2-yl)-4,6-diphenyl-1,3,5-triazine (14.11 mmol), 0.8 g of tetrakis(trphenylphosphine)palladium(0) (0.71 mmol), 5.8 g of potassium carbonate (42.34 mmol), 100 mL of toluene, 25 mL of ethanol, and 25 mL of distilled water were added, and the mixture was stirred at 130° C. for 2 hours. After completion of the reaction, the precipitated solid was washed with distilled water and methanol. The residue was purified by column chromatography to obtain 3.0 g of compound H2-250 (yield: 36%).

| | MW | M.P. |
|---|---|---|
| H2-250 | 585.7 | 225.1° C. |

### Example 23: Preparation of Compound H2-146

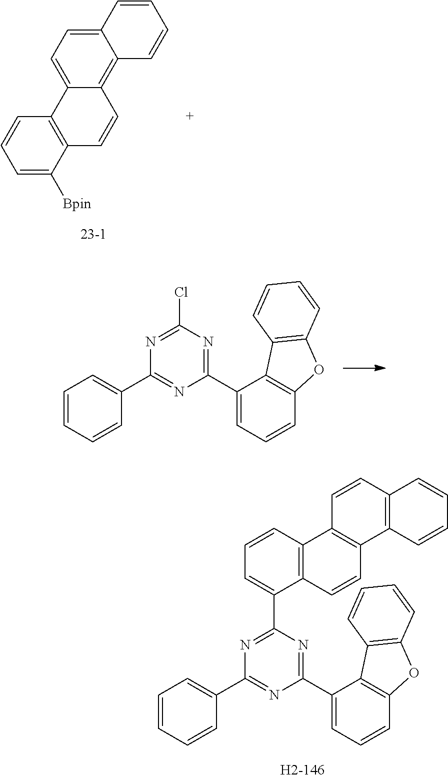

In a reaction vessel, 8.8 g of compound 23-1 (24.8 mmol), 11.6 g of 2-chloro-4-(dibenzo[b,d]furan-1-yl)-6-phenyl-1,3,5-triazine (32.3 mmol), 1.4 g of tetrakis(triphenylphosphine)palladium(0) (1.24 mmol), 6.9 g of potassium carbonate (49.68 mmol), 125 mL of toluene, 31 mL of ethanol, and 41 mL of distilled water were added, and the mixture was stirred at 130° C. for 2 hours. After completion of the reaction, the precipitated solid was washed with distilled water and methanol. The residue was purified by column chromatography to obtain 10.4 g of compound H2-146 (yield: 76%).

| | MW | M.P. |
|---|---|---|
| H2-146 | 549.6 | 281° C. |

### Example 24: Preparation of Compound H2-247

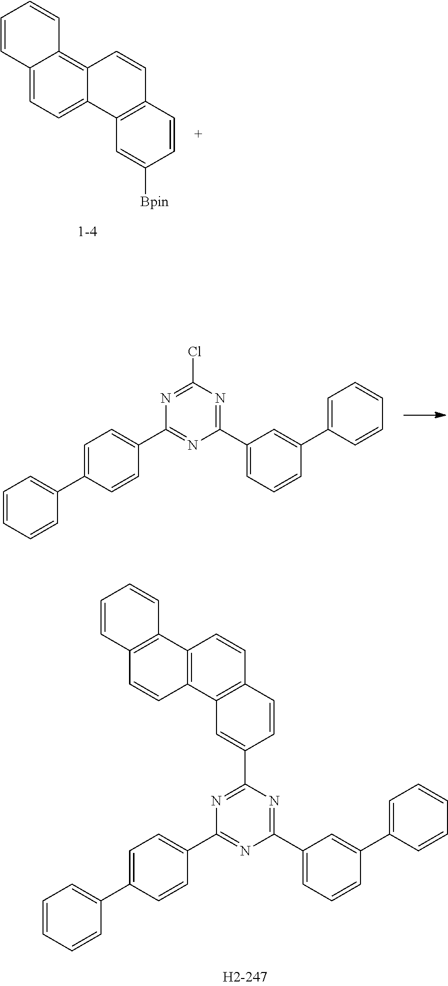

In a reaction vessel, 6.75 g of compound 1-4 (19.1 mmol), 8.0 g of 2-([1,1′-biphenyl]-3-yl)-4-([1,1′-biphenyl]-4-yl)-6-chloro-1,3,5-triazine (19.1 mmol), 0.44 g of tetrakis(triphenylphosphine)palladium(0) (0.38 mmol), 3.9 g of potassium carbonate (28.6 mmol), 240 mL of toluene, 8 mL of ethanol, and 8 mL of distilled water were added, and the mixture was stirred at 130° C. for 4 hours. After completion of the reaction, the precipitated solid was washed with distilled water and methanol. The residue was purified by column chromatography to obtain 8.1 g of compound H2-247 (yield: 70%).

| | MW | M.P. |
|---|---|---|
| H2-247 | 611.7 | 321° C. |

Hereinafter, the properties of the organic electroluminescent device (OLED) according to one embodiment of the present disclosure will be explained. However, the following examples merely illustrate the properties of an OLED according to the present disclosure in detail, but the present disclosure is not limited to the following examples.

### Device Examples 1 to 13: Producing an OLED Depositing a Plurality of Host Materials According to the Present Disclosure as Hosts

OLEDs according to the present disclosure were produced. A transparent electrode indium tin oxide (ITO) thin film (10 Ω/sq) on a glass substrate for an OLED (GEOMATEC CO., LTD., Japan) was subjected to an ultrasonic washing with acetone and isopropyl alcohol, sequentially, and then was stored in isopropyl alcohol. The ITO substrate was then mounted on a substrate holder of a vacuum vapor deposition apparatus. Compound H-1 shown in Table 3 below was introduced into a cell of the vacuum vapor deposition apparatus, and compound HT-1 shown in Table 3 below was introduced into another cell of the vacuum vapor deposition apparatus. The two materials were evaporated at different rates, and compound HI-1 was deposited in a doping amount of 3 wt % based on the total amount of compound H-1 and compound HT-1 to form a first hole injection layer having a thickness of 10 nm on the ITO substrate. Next, compound HT-1 was deposited on the first hole injection layer to form a first hole transport layer having a thickness of 80 nm. Compound HT-2 was then introduced into another cell of the vacuum vapor deposition apparatus and was evaporated by applying an electric current to the cell, thereby forming a second hole transport layer having a thickness of 60 nm on the first hole transport layer. After forming the hole injection layer and the hole transport layers, a light-emitting layer was formed thereon as follows: The first host compound and the second host compound shown in Table 1 below were introduced into two cells of the vacuum vapor depositing apparatus as hosts, and compound D-39 was introduced into another cell as a dopant. The two host materials were evaporated at a rate of 1:1 and the dopant material was simultaneously evaporated at a different rate, and the dopant was deposited in a doping amount of 3 wt % based on the total amount of the hosts and the dopant to form a light-emitting layer having a thickness of 40 nm on the second hole transport layer. Compound ETL-1 and compound EIL-1 were evaporated in a weight ratio of 50:50 to form an electron transport layer having a thickness of 35 nm on the light-emitting layer. After depositing compound EIL-1 as an electron injection layer having a thickness of 2 nm on the electron transport layer, an Al cathode having a thickness of 80 nm was deposited on the electron injection layer by another vacuum vapor deposition apparatus. Thus, an OLED was produced. All the materials used for producing the OLED were purified by vacuum sublimation at 10⁻⁶ torr.

### Comparative Examples 1 to 9: Producing an OLED Comprising a Comparative Compound as a Host

OLEDs were produced in the same manner as in Device Examples 1 to 13, except that the first host compound shown in Table 1 below was used alone as a single host material.

The driving voltage, luminous efficiency, and light-emitting color at a luminance of 1,000 nit, and the time taken for luminance to decrease from 100% to 95% (lifetime; T95) at a luminance of 5,000 nit of the OLEDs produced in Device Examples 1 to 13, and Comparative Examples 1 to 9 are provided in Table 1 below.

TABLE 1

| | | | Driving | Luminous | Light- | Lifetime |
|---|---|---|---|---|---|---|
| | First | Second | Voltage | Efficiency | Emitting | (T95) |
| | Host | Host | [V] | [cd/A] | Color | [hr] |
| Comparative | H1-45 | — | 3.7 | 8.6 | Red | 8.3 |
| Example 1 | | | | | | |
| Comparative | H1-15 | — | 4.3 | 8.4 | Red | 14.1 |
| Example 2 | | | | | | |
| Comparative | H1-27 | — | 3.7 | 8.2 | Red | 4.8 |
| Example 3 | | | | | | |
| Comparative | H1-122 | — | 4.0 | 9.8 | Red | 9.2 |
| Example 4 | | | | | | |
| Comparative | H1-124 | — | 3.7 | 9.4 | Red | 6.8 |
| Example 5 | | | | | | |
| Comparative | H1-47 | — | 4.3 | 7.4 | Red | 10.5 |
| Example 6 | | | | | | |
| Comparative | H1-102 | — | 4.4 | 5.9 | Red | 4.3 |
| Example 7 | | | | | | |
| Comparative | H1-23 | — | 4.4 | 9.6 | Red | 6.8 |
| Example 8 | | | | | | |
| Comparative | H1-80 | — | 4.4 | 8.9 | Red | 9.6 |
| Example 9 | | | | | | |
| Device | H1-15 | H2-29 | 3.3 | 35.2 | Red | 446 |
| Example 1 | | | | | | |
| Device | H1-45 | H2-29 | 3.0 | 35.3 | Red | 508 |
| Example 2 | | | | | | |
| Device | H1-27 | H2-29 | 3.1 | 34.6 | Red | 764 |
| Example 3 | | | | | | |
| Device | H1-124 | H2-29 | 3.1 | 36.2 | Red | 344 |
| Example 4 | | | | | | |
| Device | H1-47 | H2-29 | 3.1 | 36.5 | Red | 430 |
| Example 5 | | | | | | |
| Device | H1-102 | H2-29 | 3.2 | 34.4 | Red | 377 |
| Example 6 | | | | | | |
| Device | H1-23 | H2-29 | 3.5 | 35.8 | Red | 279 |
| Example 7 | | | | | | |
| Device | H1-80 | H2-29 | 3.4 | 36.0 | Red | 389 |
| Example 8 | | | | | | |
| Device | H1-103 | H2-29 | 3.0 | 36.5 | Red | 724 |
| Example 9 | | | | | | |
| Device | H1-9 | H2-29 | 3.1 | 34.8 | Red | 570 |
| Example 10 | | | | | | |
| Device | H1-15 | H2-146 | 3.2 | 37.3 | Red | 530 |
| Example 11 | | | | | | |
| Device | H1-102 | H2-146 | 3.0 | 35.4 | Red | 444 |
| Example 12 | | | | | | |
| Device | H1-113 | H2-146 | 3.0 | 37.6 | Red | 526 |
| Example 13 | | | | | | |

### Device Example 14: Producing an OILED Depositing a Compound According to the Present Disclosure as a Host

An OLED was produced in the same manner as in Device Example 1, except that compound H2-29 was used alone as a single host of the light-emitting layer.

### Comparative Example 10: Producing an OILED Comprising a Comparative Compound as a Host

An OLED was produced in the same manner as in Device Example 14, except that compound CBP was used alone as a single host of the light-emitting layer.

The driving voltage, luminous efficiency, and light-emitting color at a luminance of 1,000 nit, and the time taken for luminance to decrease from 100% to 95% (lifetime; T95) at a luminance of 5,000 nit of the OLED produced in Device Example 14 and Comparative Example 10 are provided in Table 2 below.

TABLE 2

| | | | | | Life- |
|---|---|---|---|---|---|
| | | Driving | Luminous | Light- | time |
| | | Voltage | Efficiency | Emitting | (T95) |
| | Host | [V] | [cd/A] | Color | [hr] |
| Device | H2-29 | 3.7 | 30.6 | Red | 182 |
| Example 14 | | | | | |
| Comparative | CBF | 9.0 | 14.3 | Red | 0.31 |
| Example 10 | | | | | |

From Tables 1 and 2 above, it can be confirmed that the compounds according to the present disclosure have a benefit as host materials for an OLED. In addition, it can be also confirmed that the OLED comprising the specific combination of compounds of the present disclosure as host materials provides higher luminous efficiency, and especially improved lifetime properties, as compared with an OLED using a single host material (Comparative Examples 1 to 10).

The compounds used in the Device Examples and the Comparative Examples are shown in Table 3 below.

TABLE 3
Hole Injection Layer/Hole Transport Layer

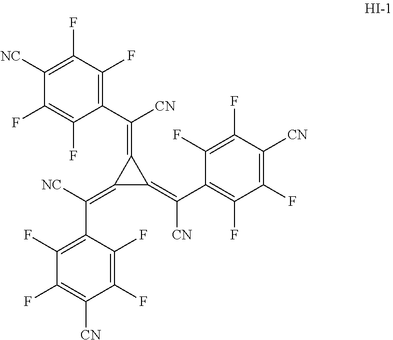 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F]

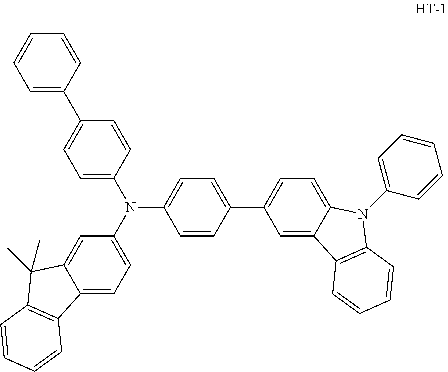 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21]

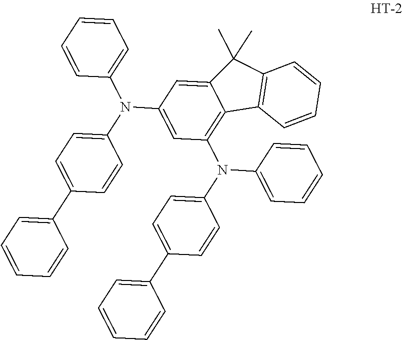 [SMILES: CC1(C)c2ccccc2-c2c(N(c3ccccc3)c3ccc(-c4ccccc4)cc3)cc(N(c3ccccc3)c3ccc(-c4ccccc4)cc3)cc21]

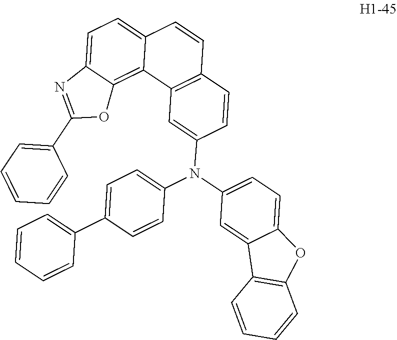 [SMILES: c1ccc(-c2ccc(N(c3ccc4oc5ccccc5c4c3)c3ccc4ccc5ccc6nc(-c7ccccc7)oc6c5c4c3)cc2)cc1]

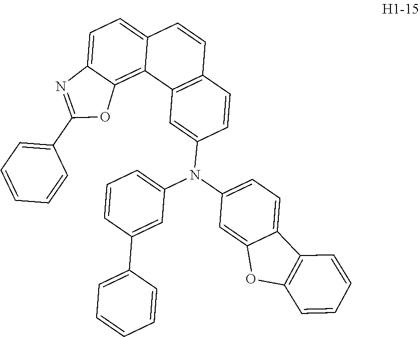 [SMILES: c1ccc(-c2cccc(N(c3ccc4c(c3)oc3ccccc34)c3ccc4ccc5ccc6nc(-c7ccccc7)oc6c5c4c3)c2)cc1]

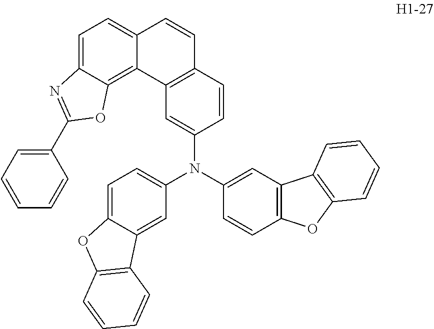 [SMILES: c1ccc(-c2nc3ccc4ccc5ccc(N(c6ccc7oc8ccccc8c7c6)c6ccc7oc8ccccc8c7c6)cc5c4c3o2)cc1]

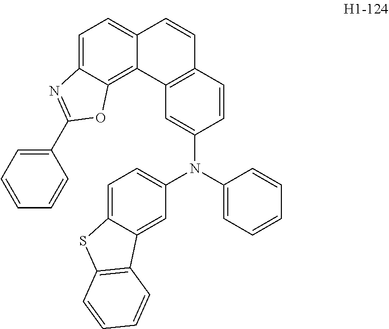 [SMILES: c1ccc(-c2nc3ccc4ccc5ccc(N(c6ccccc6)c6ccc7sc8ccccc8c7c6)cc5c4c3o2)cc1]

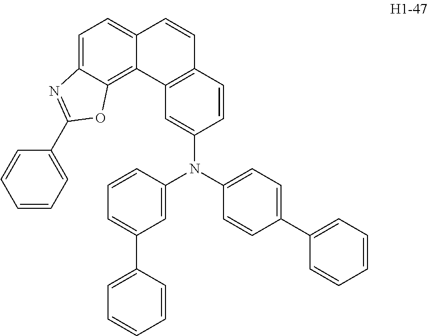 [SMILES: c1ccc(-c2ccc(N(c3cccc(-c4ccccc4)c3)c3ccc4ccc5ccc6nc(-c7ccccc7)oc6c5c4c3)cc2)cc1]

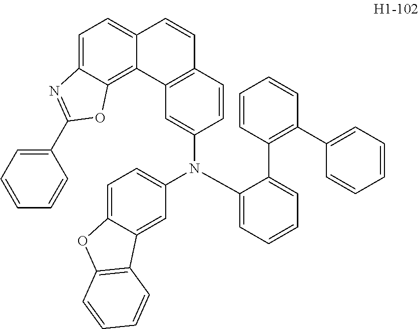 [SMILES: c1ccc(-c2nc3ccc4ccc5ccc(N(c6ccc7oc8ccccc8c7c6)c6ccccc6-c6ccccc6-c6ccccc6)cc5c4c3o2)cc1]

Light-Emitting Layer

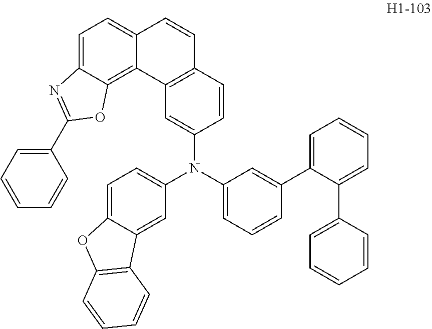 [SMILES: c1ccc(-c2nc3ccc4ccc5ccc(N(c6cccc(-c7ccccc7-c7ccccc7)c6)c6ccc7oc8ccccc8c7c6)cc5c4c3o2)cc1]

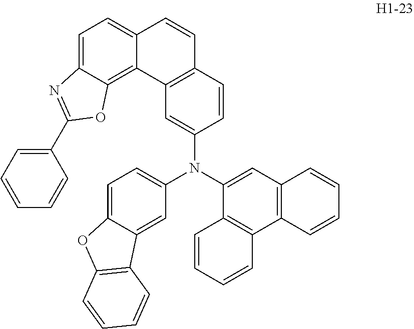 [SMILES: c1ccc(-c2nc3ccc4ccc5ccc(N(c6ccc7oc8ccccc8c7c6)c6cc7ccccc7c7ccccc67)cc5c4c3o2)cc1]

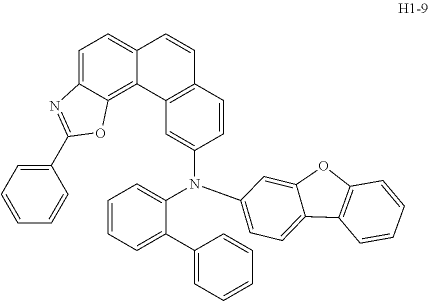 [SMILES: c1ccc(-c2nc3ccc4ccc5ccc(N(c6ccc7c(c6)oc6ccccc67)c6ccccc6-c6ccccc6)cc5c4c3o2)cc1]

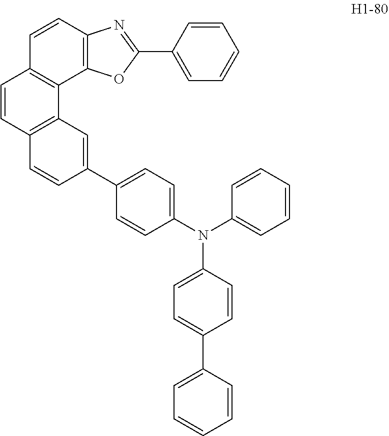 [SMILES: c1ccc(-c2ccc(N(c3ccccc3)c3ccc(-c4ccc5ccc6ccc7nc(-c8ccccc8)oc7c6c5c4)cc3)cc2)cc1]

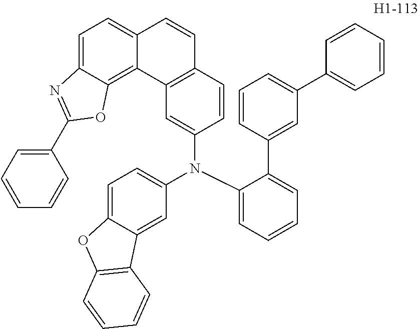 [SMILES: c1ccc(-c2cccc(-c3ccccc3N(c3ccc4oc5ccccc5c4c3)c3ccc4ccc5ccc6nc(-c7ccccc7)oc6c5c4c3)c2)cc1]

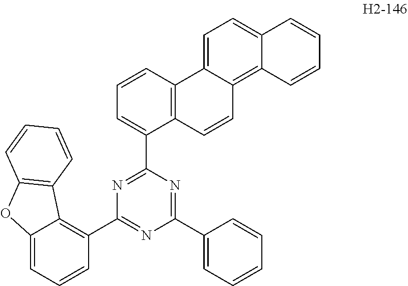 [SMILES: c1ccc(-c2nc(-c3cccc4c3ccc3c5ccccc5ccc43)nc(-c3cccc4oc5ccccc5c34)n2)cc1]

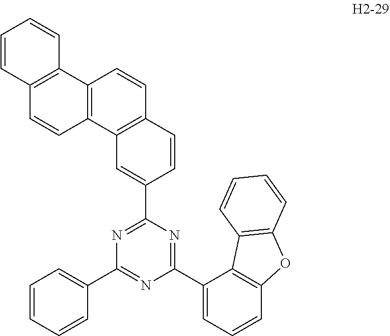 [SMILES: c1ccc(-c2nc(-c3ccc4ccc5c6ccccc6ccc5c4c3)nc(-c3cccc4oc5ccccc5c34)n2)cc1]

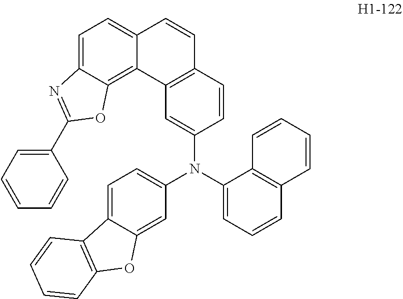 [SMILES: c1ccc(-c2nc3ccc4ccc5ccc(N(c6ccc7c(c6)oc6ccccc67)c6cccc7ccccc67)cc5c4c3o2)cc1]

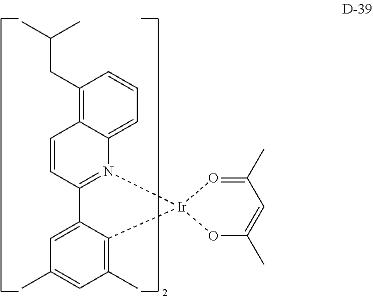

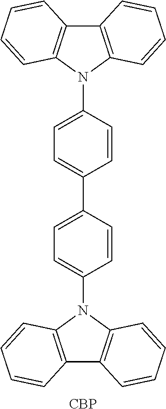

Electron Transport Layer/Electron Injection Layer

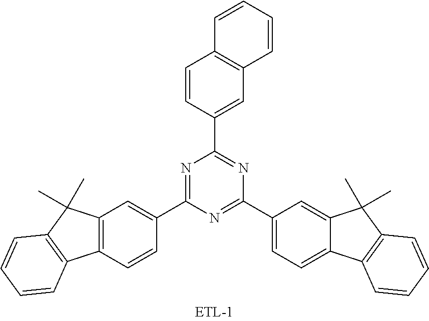 [SMILES: CC1(C)c2ccccc2-c2ccc(-c3nc(-c4ccc5c(c4)C(C)(C)c4ccccc4-5)nc(-c4ccc5ccccc5c4)n3)cc21]

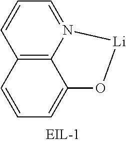

1. A plurality of host materials comprising a first host material comprising a compound represented by the following formula 1, and a second host material comprising a compound represented by the following formula 2: 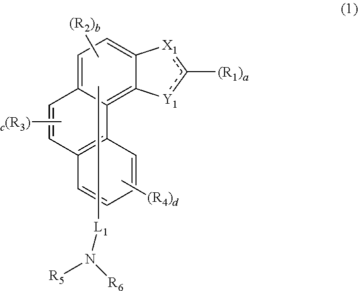
wherein
X₁ and Y₁, each independently, represent —N═, —NR₇—, —O—, or —S—, with the proviso that any one of X₁ and Y₁ represents —N═, and the other of X₁ and Y represents —NR₇—, —O—, or —S—;
R₁ represents a substituted or unsubstituted (C6-C30)aryl, or a substituted or unsubstituted (3- to 30-membered)heteroaryl;
R₂ to R₇, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring group of a (C3-C30) aliphatic ring(s) and a (C6-C30) aromatic ring(s), a substituted or unsubstituted mono- or di-(C1-C30)alkylamino, a substituted or unsubstituted mono- or di-(C2-C30)alkenylamino, a substituted or unsubstituted (C1-C30)alkyl(C2-C30)alkenylamino, a substituted or unsubstituted mono- or di-(C6-C30)arylamino, a substituted or unsubstituted (C1-C30)alkyl(C6-C30)arylamino, a substituted or unsubstituted mono- or di-(3- to 30-membered)heteroarylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, or a substituted or unsubstituted (C6-C30)aryl(3- to 30-membered)heteroarylamino; or may be linked to an adjacent substituent to form a ring(s);
L₁ represents a single bond, a substituted or unsubstituted (C6-C30)arylene, or a substituted or unsubstituted (3- to 30-membered)heteroarylene; and
a represents 1; b and c, each independently, represent 1 or 2; d represents an integer of 1 to 4; where if each of b to d is an integer of 2 or more, each of R₂ to R₄ may be the same or different; 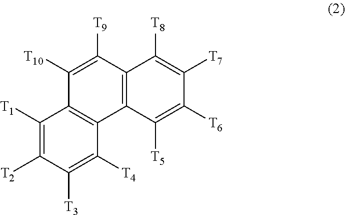
wherein
T₅ and T₆ are linked to each other to form a ring represented by the following formula 3; or T₇ and T₈ are linked to each other to form a ring represented by the following formula 3; or T₅ and T₆ are linked to each other to form a ring represented by the following formula 3, and T₇ and T₈ are also linked to each other to form a ring represented by the following formula 3: 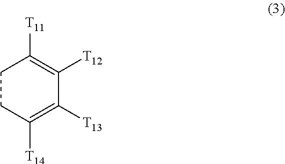
in formulas 2 and 3,
T₁ to T₄, T₉ to T₁₄, and T₅ to T₆ not forming a ring, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arysilyl, a substituted or unsubstituted tri(C6-C30)arylsilyl, a substituted or unsubstituted fused ring group of a (C3-C30) aliphatic ring(s) and a (C6-C30) aromatic ring(s), a substituted or unsubstituted mono- or di-(C1-C30)alkylamino, a substituted or unsubstituted mono- or di-(C2-C30)alkenylamino, a substituted or unsubstituted (C1-C30)alkyl(C2-C30)alkenylamino, a substituted or unsubstituted mono- or di-(C6-C30)arylamino, a substituted or unsubstituted (C1-C30)alkyl(C6-C30)arylamino, a substituted or unsubstituted mono- or di-(3- to 30-membered)heteroarylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, a substituted or unsubstituted (C6-C30)aryl(3- to 30-membered)heteroarylamino, or -L₂-Ar₂; with the proviso that at least one of T₁ to T₁₄ represents -L₂-Ar₂;
L₂, each independently, represents a single bond, a substituted or unsubstituted (C6-C30)arylene, or a substituted or unsubstituted (3- to 30-membered)heteroarylene;
Ar₂, each independently, represents a substituted or unsubstituted (C6-C30)aryl, or a substituted or unsubstituted (3- to 30-membered)heteroaryl; [symbol]
represents a fusion site with formula 2; and
the heteroaryl contains at least one heteroatom selected from the group consisting of B, N, O, S, Si, and P.

2. The plurality of host materials according to claim 1, wherein the substituents of the substituted alkyl, the substituted aryl, the substituted arylene, the substituted heteroaryl, the substituted heteroarylene, the substituted cycloalkyl, the substituted alkoxy, the substituted trialkylsilyl, the substituted dialkylarylsilyl, the substituted alkyldiarylsilyl, the substituted triarylsilyl, the substituted fused ring group of a aliphatic ring(s) and a aromatic ring(s), the substituted mono- or di-alkylamino, the substituted mono- or di-alkenylamino, the substituted alkylalkenylamino, the substituted mono- or di-arylamino, the substituted alkylarylamino, the substituted mono- or di-heteroarylamino, the substituted alkylheteroarylamino, the substituted alkenylarylamino, the substituted alkenylheteroarylamino, and the substituted arylheteroarylamino, each independently, are at least one selected from the group consisting of deuterium; a halogen; a cyano; a carboxyl; a nitro; a hydroxyl; a phosphine oxide; a (C1-C30)alkyl; a halo(C1-C30)alkyl; a (C2-C30)alkenyl; a (C2-C30)alkynyl; a (C1-C30)alkoxy; a (C1-C30)alkylthio; a (C3-C30)cycloalkyl; a (C3-C30)cycloalkenyl; a (3- to 7-membered)heterocycloalkyl; a (C6-C30)aryloxy; a (C6-C30)arylthio; a (3- to 30-membered)heteroaryl unsubstituted or substituted with at least one of a (C1-C30)alkyl(s) and a (C6-C30)aryl(s); a (C6-C30)aryl unsubstituted or substituted with at least one of deuterium, a cyano(s), a (C1-C30)alkyl(s), a (C3-C30)cycloalkyl(s), a tri(C1-C30)alkylsilyl(s), and a (3- to 30-membered)heteroaryl(s); a tri(C1-C30)alkylsilyl; a tri(C6-C30)arylsilyl; a di(C1-C30)alkyl(C6-C30)arylsilyl; a (C1-C30)alkyldi(C6-C30)arysilyl; a fused ring group of a (C3-C30) aliphatic ring(s) and a (C6-C30) aromatic ring(s); an amino; a mono- or di-(C1-C30)alkylamino; a mono- or di-(C2-C30)alkenylamino; a (C1-C30)alkyl(C2-C30)alkenylamino; a mono- or di-(C6-C30)arylamino; a (C1-C30)alkyl(C6-C30)arylamino; a mono- or di-(3- to 30-membered)heteroarylamino; a (C1-C30)alkyl(3- to 30-membered)heteroarylamino; a (C2-C30)alkenyl(C6-C30)arylamino; a (C2-C30)alkenyl(3- to 30-membered)heteroarylamino; a (C6-C30)aryl(3- to 30-membered)heteroarylamino; a (C1-C30)alkylcarbonyl; a (C1-C30)alkoxycarbonyl; a (C6-C30)arylcarbonyl; a (C6-C30)arylphosphine; a di(C6-C30)arylboronyl; a di(C1-C30)alkylboronyl; a (C1-C30)alkyl(C6-C30)arylboronyl; a (C6-C30)aryl(C1-C30)alkyl; and a (C1-C30)alkyl(C6-C30)aryl.

3. The plurality of host materials according to claim 1, wherein the formula 1 is represented by at least one of the following formulas 1-1 to 1-3: 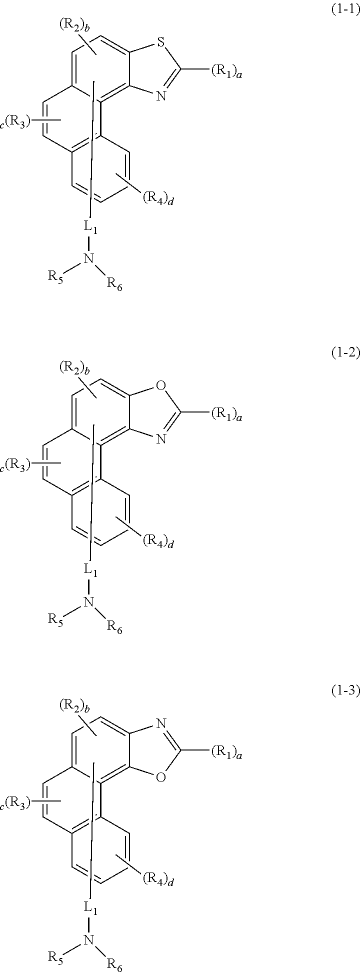
wherein,
R₁ to R₆, L₁, and a to d are as defined in claim 1.

4. The plurality of host materials according to claim 1, where in formula 1, R₁, R₅, and R₆, each independently, represent a substituted or unsubstituted phenyl, a substituted or unsubstituted naphthyl, a substituted or unsubstituted biphenyl, a substituted or unsubstituted terphenyl, a substituted or unsubstituted phenanthrenyl, a substituted or unsubstituted fluorenyl, a substituted or unsubstituted benzofluorenyl, a substituted or unsubstituted triphenylenyl, a substituted or unsubstituted spirobifluorenyl, a substituted or unsubstituted pyridyl, a substituted or unsubstituted triazinyl, a substituted or unsubstituted pyrimidinyl, a substituted or unsubstituted quinolyl, a substituted or unsubstituted quinazolinyl, a substituted or unsubstituted quinoxalinyl, a substituted or unsubstituted benzoquinazolinyl, a substituted or unsubstituted benzoquinoxalinyl, a substituted or unsubstituted benzofuropyrmidinyl, a substituted or unsubstituted carbazolyl, a substituted or unsubstituted dibenzothiophenyl, a substituted or unsubstituted benzothiophenyl, a substituted or unsubstituted dibenzofuranyl, a substituted or unsubstituted benzofuranyl, a substituted or unsubstituted naphthyridinyl, a substituted or unsubstituted benzonaphthofuranyl, or a substituted or unsubstituted benzonaphthothiophenyl.

5. The plurality of host materials according to claim 1, wherein the formula 2 is represented by at least one of the following formulas 2-1 and 2-2: 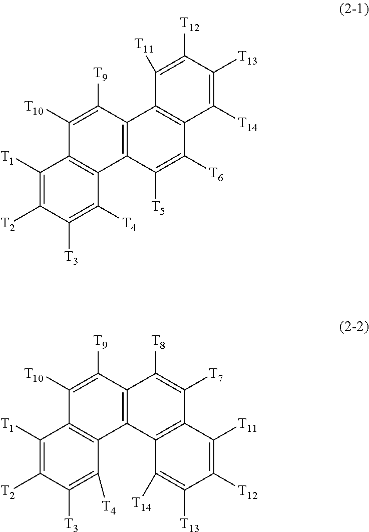
wherein,
T₁ to T₄, and T₉ to T₁₄ are as defined in claim 1, and T₅ to T₈ are defined as T₅ to T₈ not forming a ring in claim 1.

6. The plurality of host materials according to claim 1, wherein the formula 2 is represented by at least one of the following formulas: 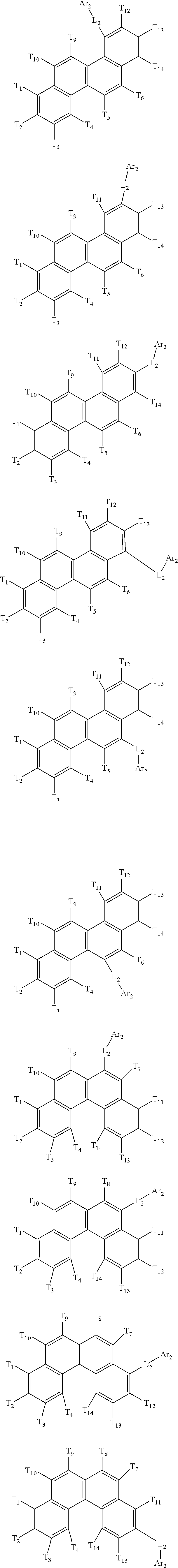 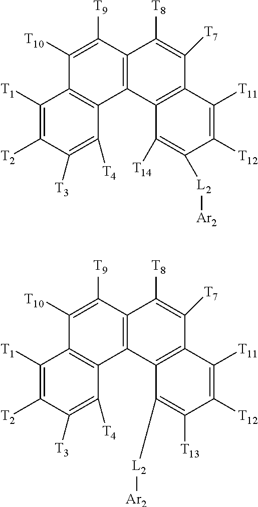
wherein,
T₁ to T₁₄, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring group of a (C3-C30) aliphatic ring(s) and a (C6-C30) aromatic ring(s), a substituted or unsubstituted mono- or di-(C1-C30)alkylamino, a substituted or unsubstituted mono- or di-(C2-C30)alkenylamino, a substituted or unsubstituted (C1-C30)alkyl(C2-C30)alkenylamino, a substituted or unsubstituted mono- or di-(C6-C30)arylamino, a substituted or unsubstituted (C1-C30)alkyl(C6-C30)arylamino, a substituted or unsubstituted mono- or di-(3- to 30-membered)heteroarylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, or a substituted or unsubstituted (C6-C30)aryl(3- to 30-membered)heteroarylamino; and
L₂ and Ar₂ are as defined in claim 1.

7. The plurality of host materials according to claim 1, wherein the compound represented by formula 1 is at least one selected from the following compounds:

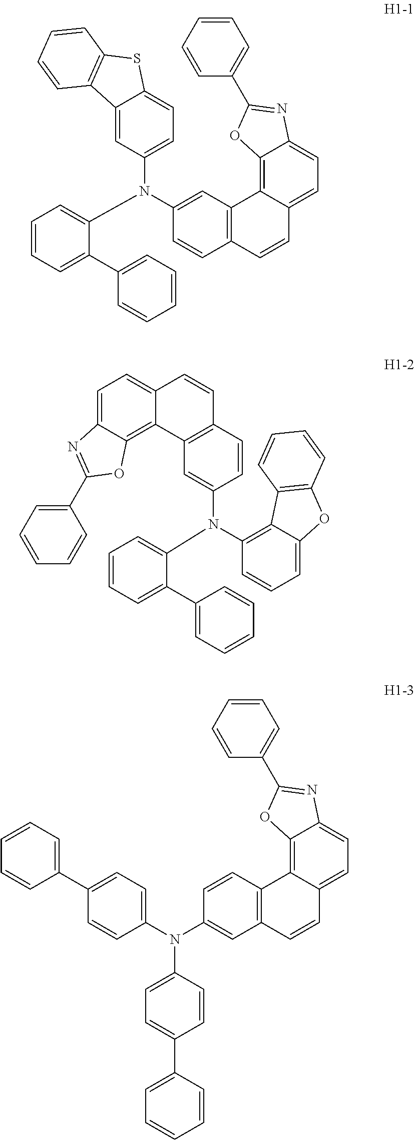

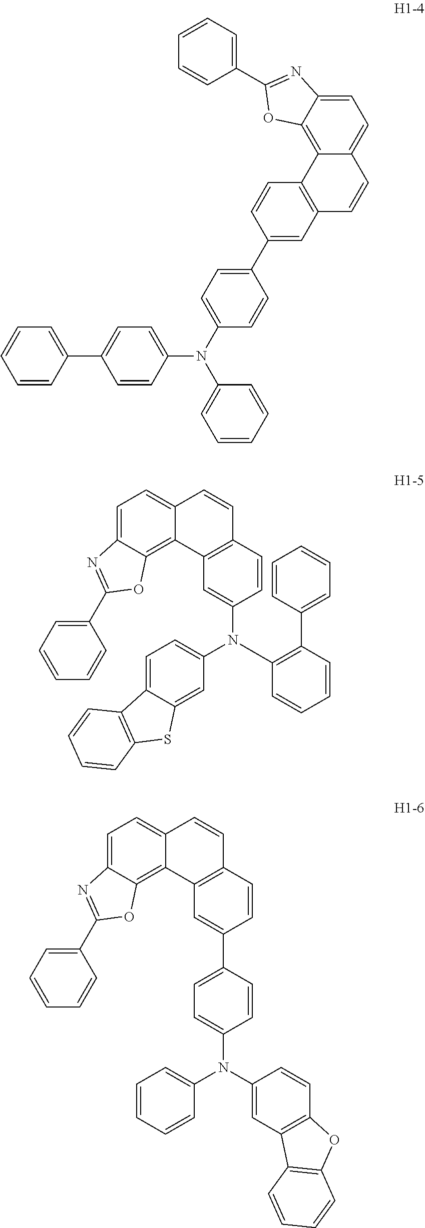 [SMILES: c1ccc(-c2ccc(N(c3ccccc3)c3ccc(-c4ccc5c(ccc6ccc7nc(-c8ccccc8)oc7c65)c4)cc3)cc2)cc1 ; c1ccc(-c2nc3ccc4ccc5ccc(N(c6ccc7c(c6)sc6ccccc67)c6ccccc6-c6ccccc6)cc5c4c3o2)cc1 ; c1ccc(-c2nc3ccc4ccc5ccc(-c6ccc(N(c7ccccc7)c7ccc8oc9ccccc9c8c7)cc6)cc5c4c3o2)cc1]

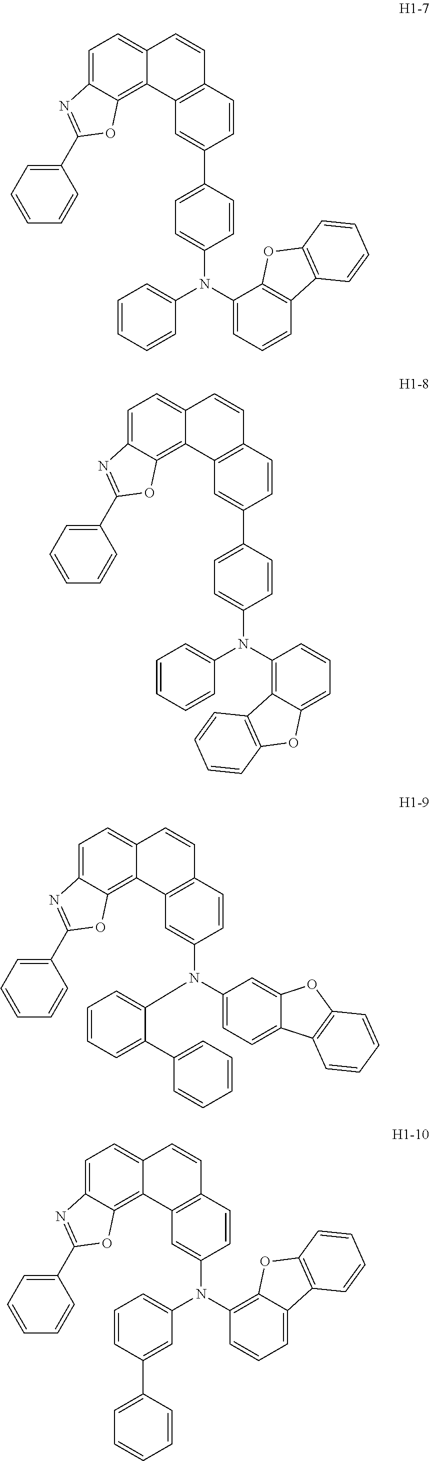

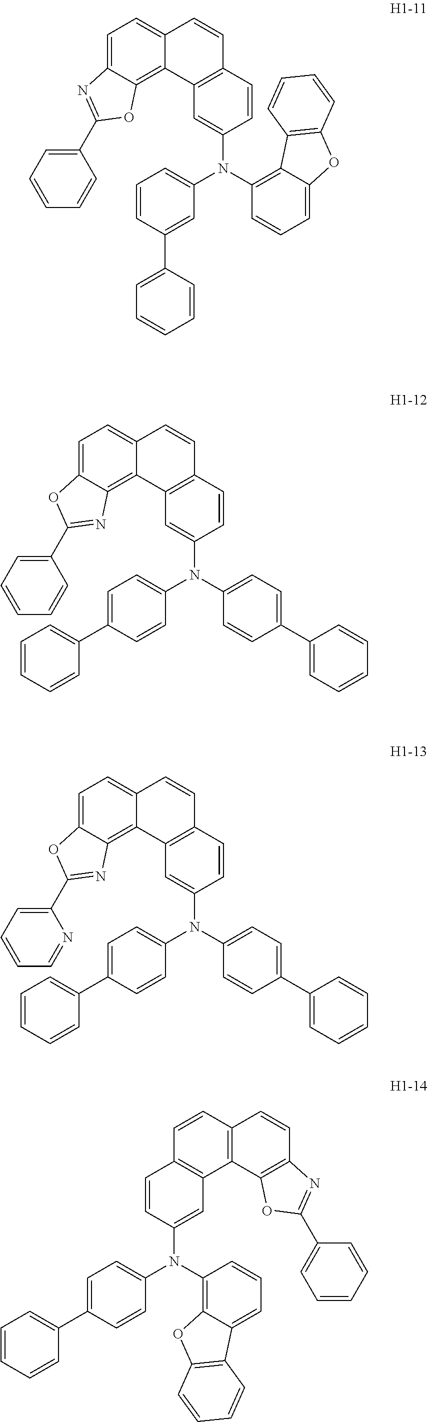 [SMILES: c1ccc(-c2cccc(N(c3ccc4ccc5ccc6nc(-c7ccccc7)oc6c5c4c3)c3cccc4oc5ccccc5c34)c2)cc1 ; c1ccc(-c2ccc(N(c3ccc(-c4ccccc4)cc3)c3ccc4ccc5ccc6oc(-c7ccccc7)nc6c5c4c3)cc2)cc1 ; c1ccc(-c2ccc(N(c3ccc(-c4ccccc4)cc3)c3ccc4ccc5ccc6oc(-c7ccccn7)nc6c5c4c3)cc2)cc1 ; c1ccc(-c2ccc(N(c3ccc4ccc5ccc6nc(-c7ccccc7)oc6c5c4c3)c3cccc4c3oc3ccccc34)cc2)cc1]

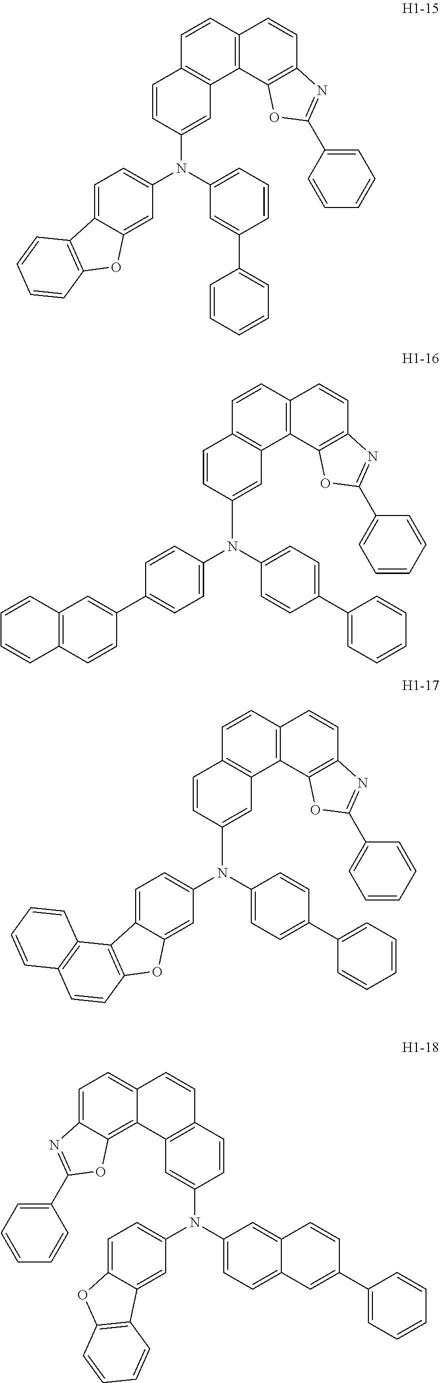 [SMILES: c1ccc(-c2cccc(N(c3ccc4c(c3)oc3ccccc34)c3ccc4ccc5ccc6nc(-c7ccccc7)oc6c5c4c3)c2)cc1 ; c1ccc(-c2ccc(N(c3ccc(-c4ccc5ccccc5c4)cc3)c3ccc4ccc5ccc6nc(-c7ccccc7)oc6c5c4c3)cc2)cc1 ; c1ccc(-c2ccc(N(c3ccc4c(c3)oc3ccc5ccccc5c34)c3ccc4ccc5ccc6nc(-c7ccccc7)oc6c5c4c3)cc2)cc1 ; c1ccc(-c2ccc3cc(N(c4ccc5oc6ccccc6c5c4)c4ccc5ccc6ccc7nc(-c8ccccc8)oc7c6c5c4)ccc3c2)cc1]

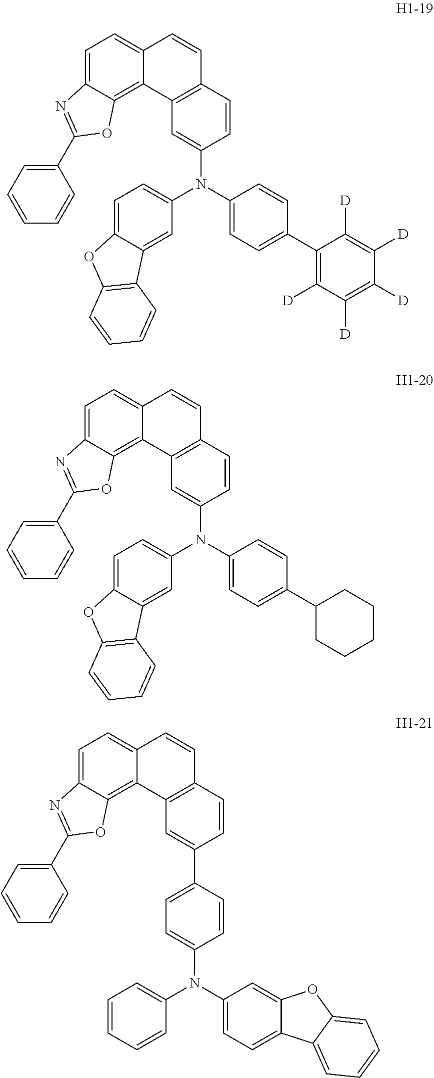

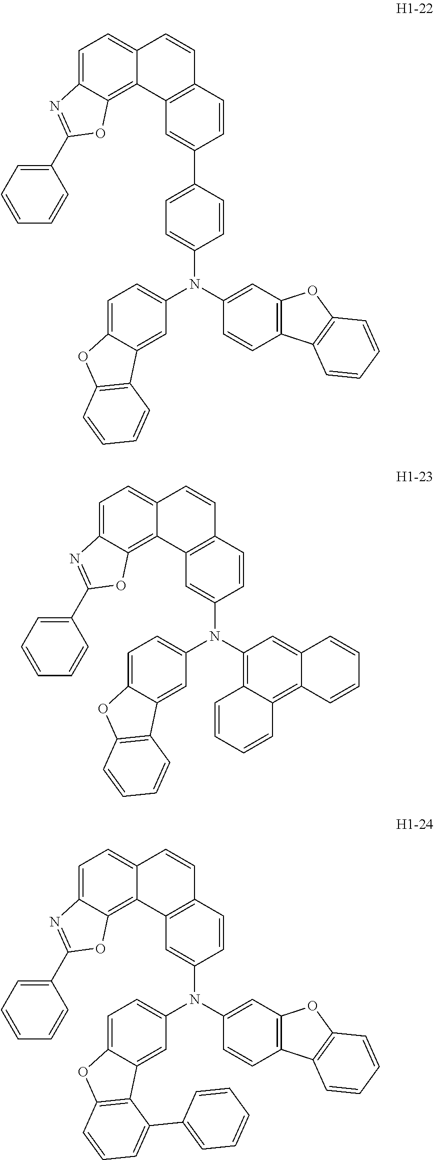

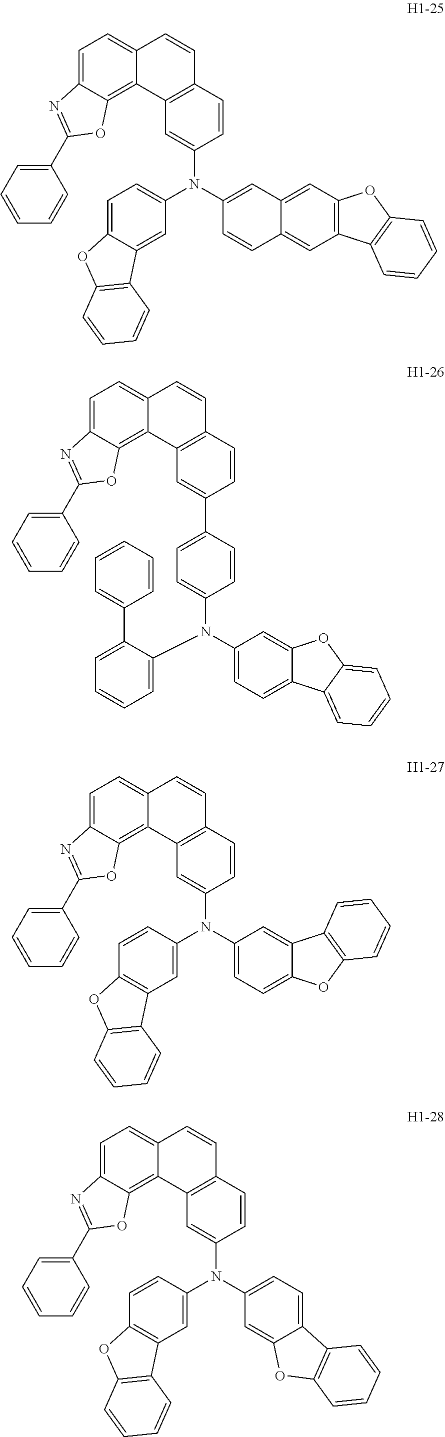

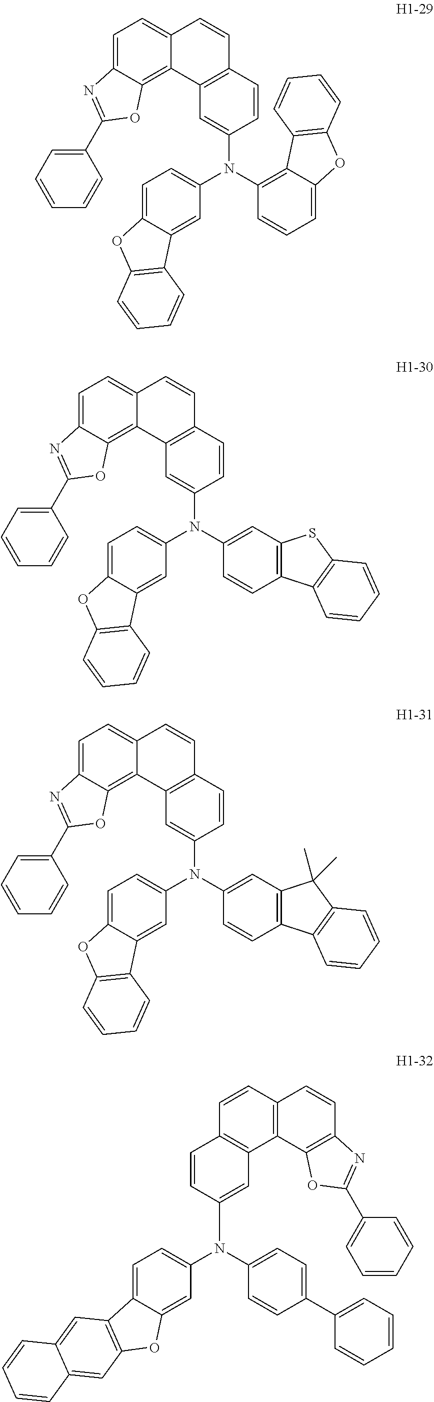

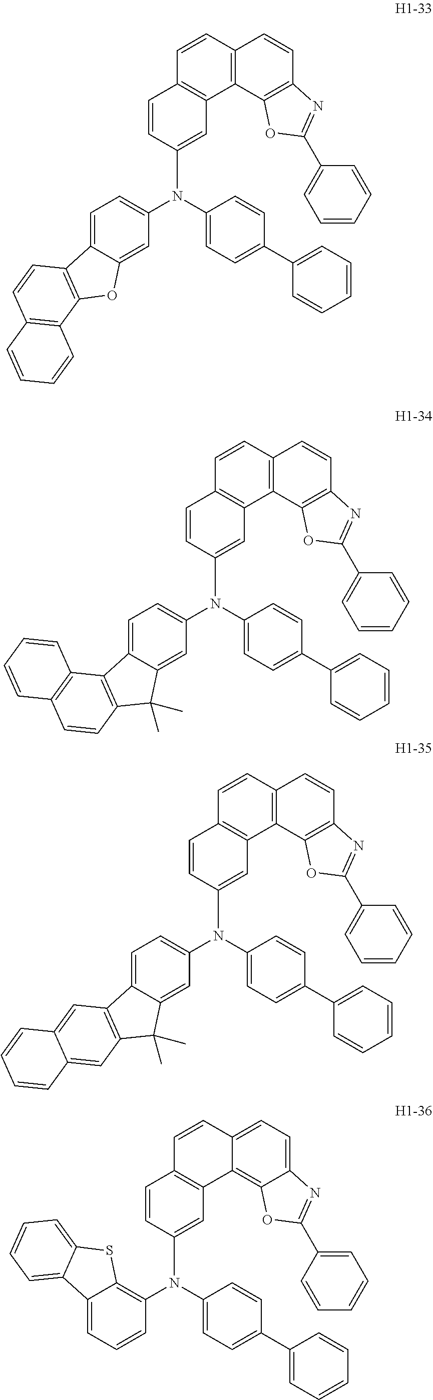 [SMILES: c1ccc(-c2ccc(N(c3ccc4c(c3)oc3c5ccccc5ccc43)c3ccc4ccc5ccc6nc(-c7ccccc7)oc6c5c4c3)cc2)cc1 ; CC1(C)c2cc(N(c3ccc(-c4ccccc4)cc3)c3ccc4ccc5ccc6nc(-c7ccccc7)oc6c5c4c3)ccc2-c2c1ccc1ccccc21 ; CC1(C)c2cc(N(c3ccc(-c4ccccc4)cc3)c3ccc4ccc5ccc6nc(-c7ccccc7)oc6c5c4c3)ccc2-c2cc3ccccc3cc21 ; c1ccc(-c2ccc(N(c3ccc4ccc5ccc6nc(-c7ccccc7)oc6c5c4c3)c3cccc4c3sc3ccccc34)cc2)cc1]

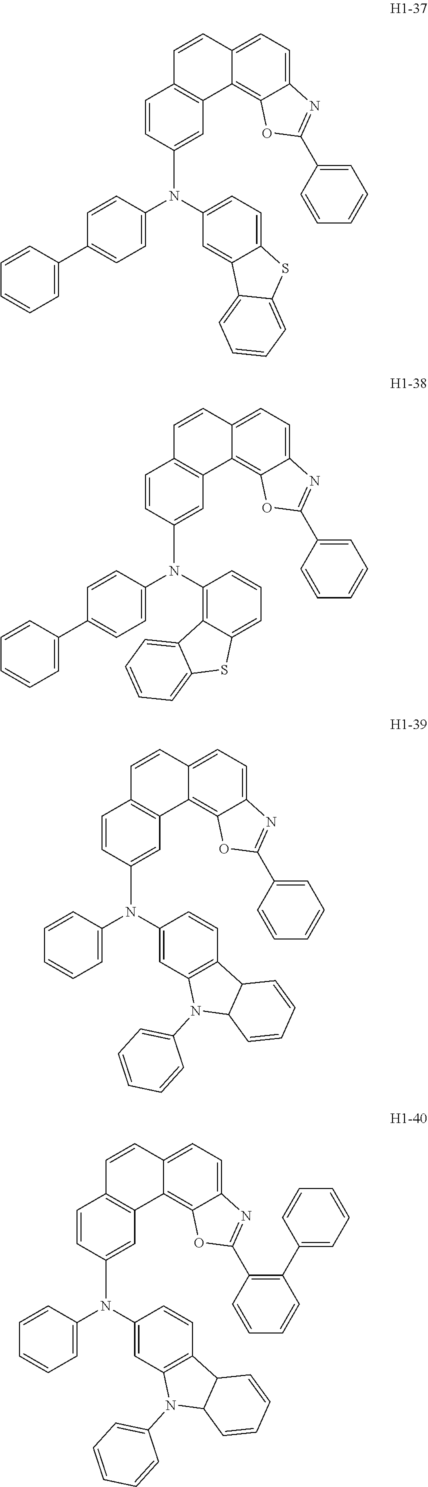 [SMILES: c1ccc(-c2ccc(N(c3ccc4sc5ccccc5c4c3)c3ccc4ccc5ccc6nc(-c7ccccc7)oc6c5c4c3)cc2)cc1 ; c1ccc(-c2ccc(N(c3ccc4ccc5ccc6nc(-c7ccccc7)oc6c5c4c3)c3cccc4sc5ccccc5c34)cc2)cc1 ; C1=CC2c3ccc(N(c4ccccc4)c4ccc5ccc6ccc7nc(-c8ccccc8)oc7c6c5c4)cc3N(c3ccccc3)C2C=C1 ; C1=CC2c3ccc(N(c4ccccc4)c4ccc5ccc6ccc7nc(-c8ccccc8-c8ccccc8)oc7c6c5c4)cc3N(c3ccccc3)C2C=C1]

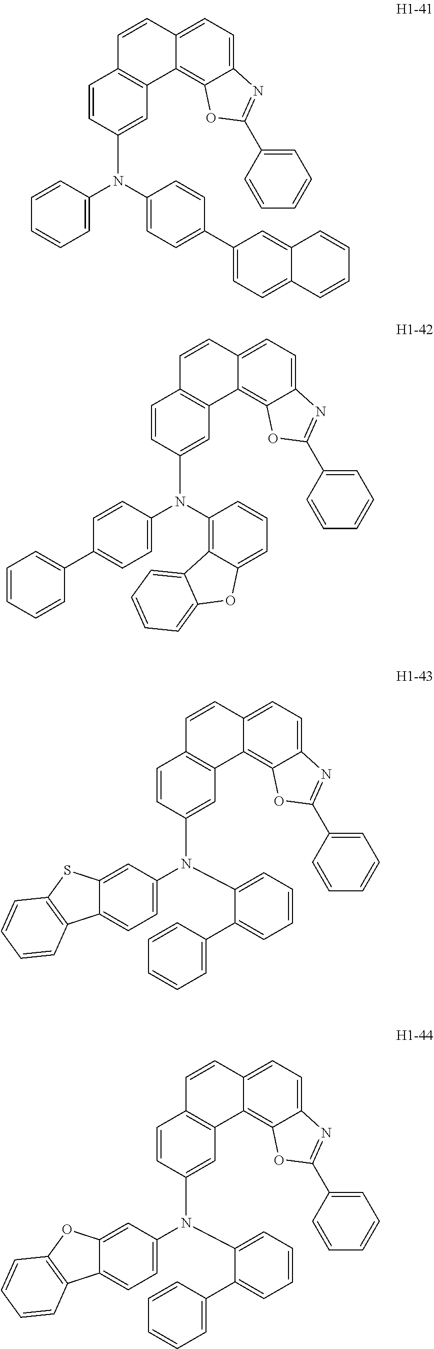

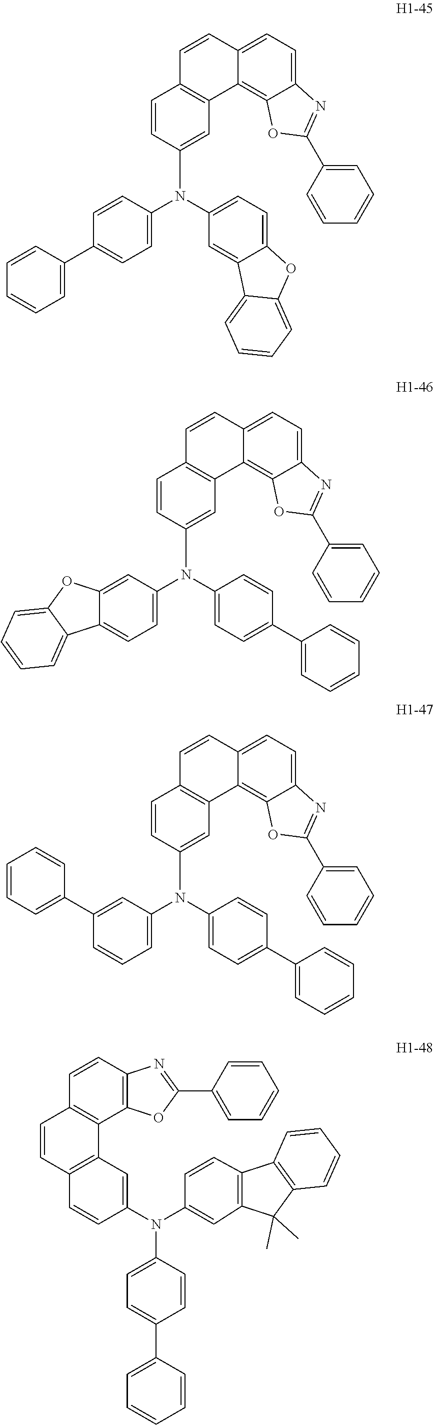 [SMILES: c1ccc(-c2ccc(N(c3ccc4oc5ccccc5c4c3)c3ccc4ccc5ccc6nc(-c7ccccc7)oc6c5c4c3)cc2)cc1 ; c1ccc(-c2ccc(N(c3ccc4c(c3)oc3ccccc34)c3ccc4ccc5ccc6nc(-c7ccccc7)oc6c5c4c3)cc2)cc1 ; c1ccc(-c2ccc(N(c3cccc(-c4ccccc4)c3)c3ccc4ccc5ccc6nc(-c7ccccc7)oc6c5c4c3)cc2)cc1 ; CC1(C)c2ccccc2-c2ccc(N(c3ccc(-c4ccccc4)cc3)c3ccc4ccc5ccc6nc(-c7ccccc7)oc6c5c4c3)cc21]

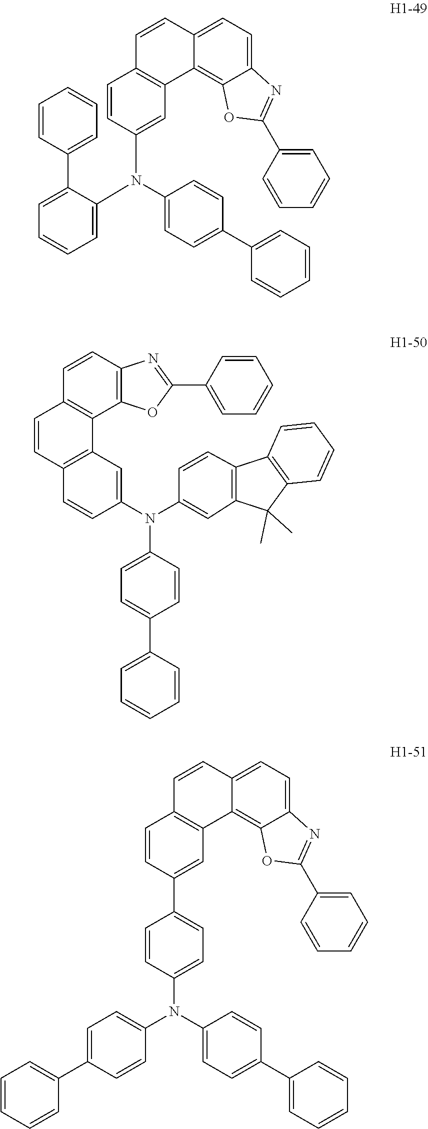 [SMILES: c1ccc(-c2ccc(N(c3ccc4ccc5ccc6nc(-c7ccccc7)oc6c5c4c3)c3ccccc3-c3ccccc3)cc2)cc1 ; CC1(C)c2ccccc2-c2ccc(N(c3ccc(-c4ccccc4)cc3)c3ccc4ccc5ccc6nc(-c7ccccc7)oc6c5c4c3)cc21 ; c1ccc(-c2ccc(N(c3ccc(-c4ccccc4)cc3)c3ccc(-c4ccc5ccc6ccc7nc(-c8ccccc8)oc7c6c5c4)cc3)cc2)cc1]

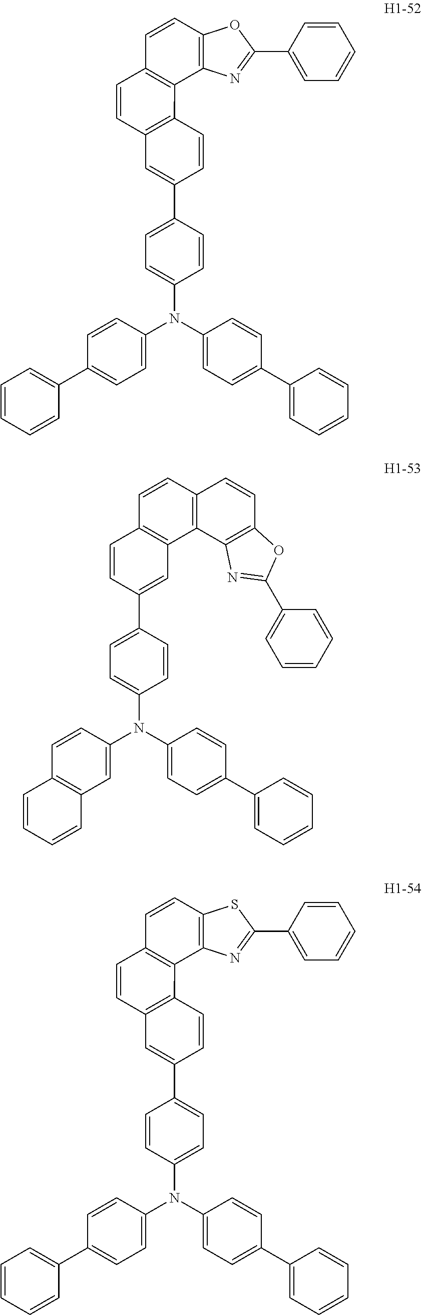 [SMILES: c1ccc(-c2ccc(N(c3ccc(-c4ccccc4)cc3)c3ccc(-c4ccc5c(ccc6ccc7oc(-c8ccccc8)nc7c65)c4)cc3)cc2)cc1 ; c1ccc(-c2ccc(N(c3ccc(-c4ccc5ccc6ccc7oc(-c8ccccc8)nc7c6c5c4)cc3)c3ccc4ccccc4c3)cc2)cc1 ; c1ccc(-c2ccc(N(c3ccc(-c4ccccc4)cc3)c3ccc(-c4ccc5c(ccc6ccc7sc(-c8ccccc8)nc7c65)c4)cc3)cc2)cc1]

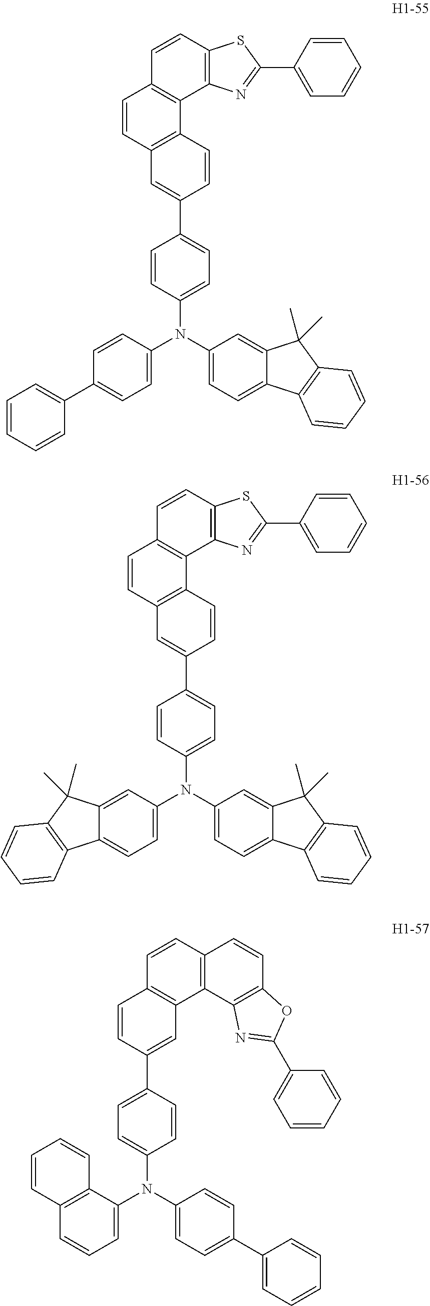 [SMILES: CC1(C)c2ccccc2-c2ccc(N(c3ccc(-c4ccccc4)cc3)c3ccc(-c4ccc5c(ccc6ccc7sc(-c8ccccc8)nc7c65)c4)cc3)cc21 ; CC1(C)c2ccccc2-c2ccc(N(c3ccc(-c4ccc5c(ccc6ccc7sc(-c8ccccc8)nc7c65)c4)cc3)c3ccc4c(c3)C(C)(C)c3ccccc3-4)cc21 ; c1ccc(-c2ccc(N(c3ccc(-c4ccc5ccc6ccc7oc(-c8ccccc8)nc7c6c5c4)cc3)c3cccc4ccccc34)cc2)cc1]

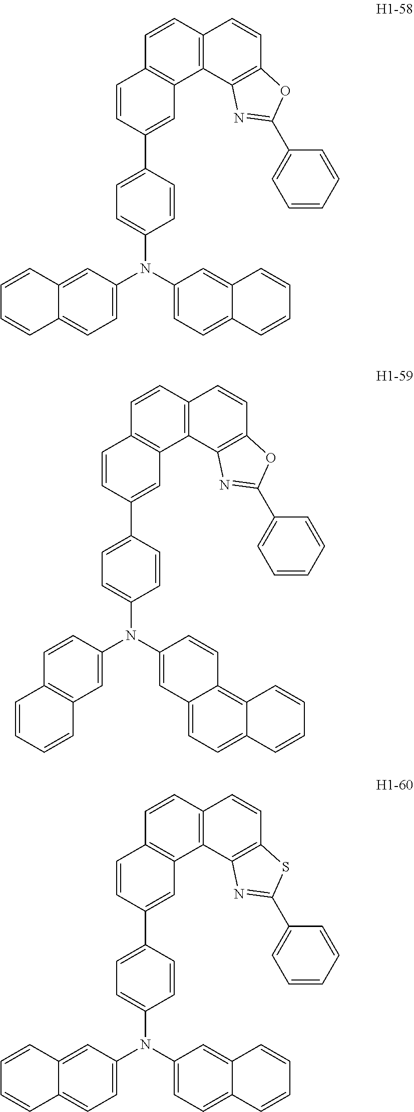

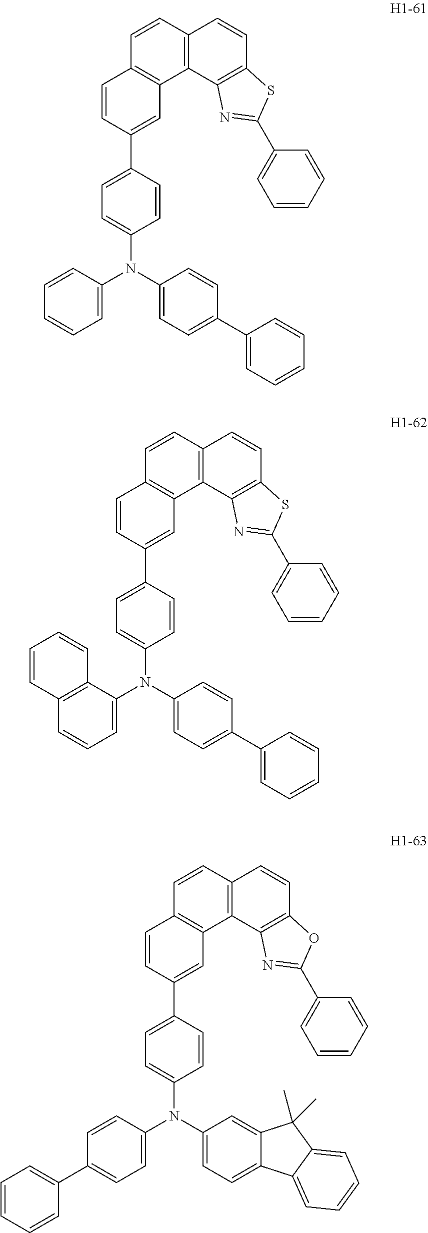 [SMILES: c1ccc(-c2ccc(N(c3ccccc3)c3ccc(-c4ccc5ccc6ccc7sc(-c8ccccc8)nc7c6c5c4)cc3)cc2)cc1 ; c1ccc(-c2ccc(N(c3ccc(-c4ccc5ccc6ccc7sc(-c8ccccc8)nc7c6c5c4)cc3)c3cccc4ccccc34)cc2)cc1 ; CC1(C)c2ccccc2-c2ccc(N(c3ccc(-c4ccccc4)cc3)c3ccc(-c4ccc5ccc6ccc7oc(-c8ccccc8)nc7c6c5c4)cc3)cc21]

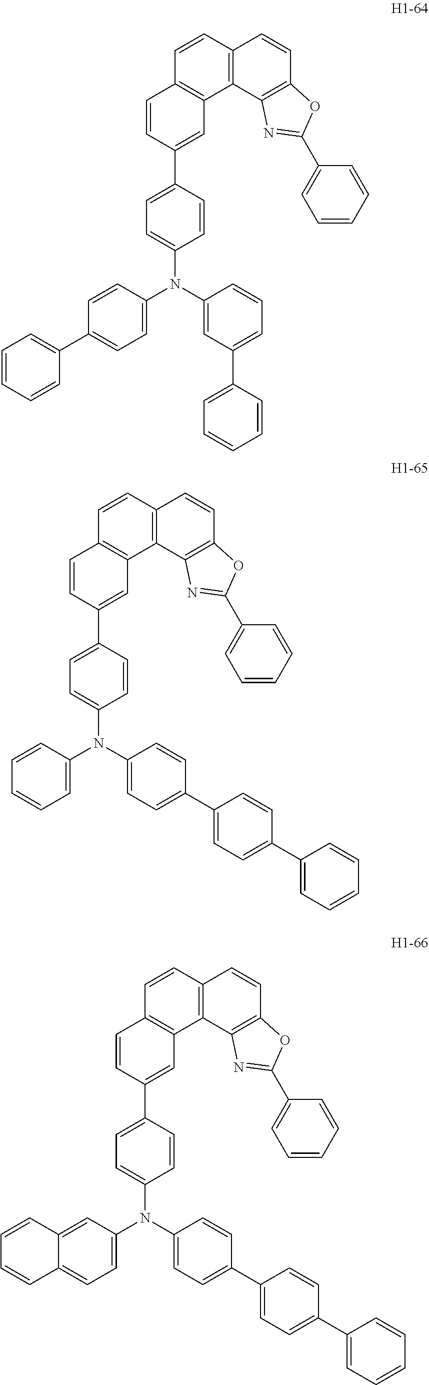 [SMILES: c1ccc(-c2ccc(N(c3ccc(-c4ccc5ccc6ccc7oc(-c8ccccc8)nc7c6c5c4)cc3)c3cccc(-c4ccccc4)c3)cc2)cc1 ; c1ccc(-c2ccc(-c3ccc(N(c4ccccc4)c4ccc(-c5ccc6ccc7ccc8oc(-c9ccccc9)nc8c7c6c5)cc4)cc3)cc2)cc1 ; c1ccc(-c2ccc(-c3ccc(N(c4ccc(-c5ccc6ccc7ccc8oc(-c9ccccc9)nc8c7c6c5)cc4)c4ccc5ccccc5c4)cc3)cc2)cc1]

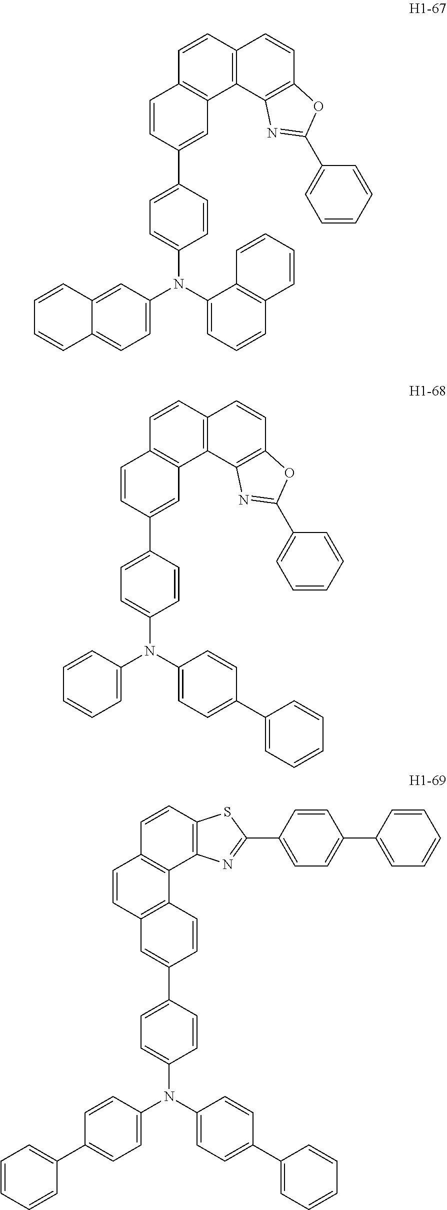

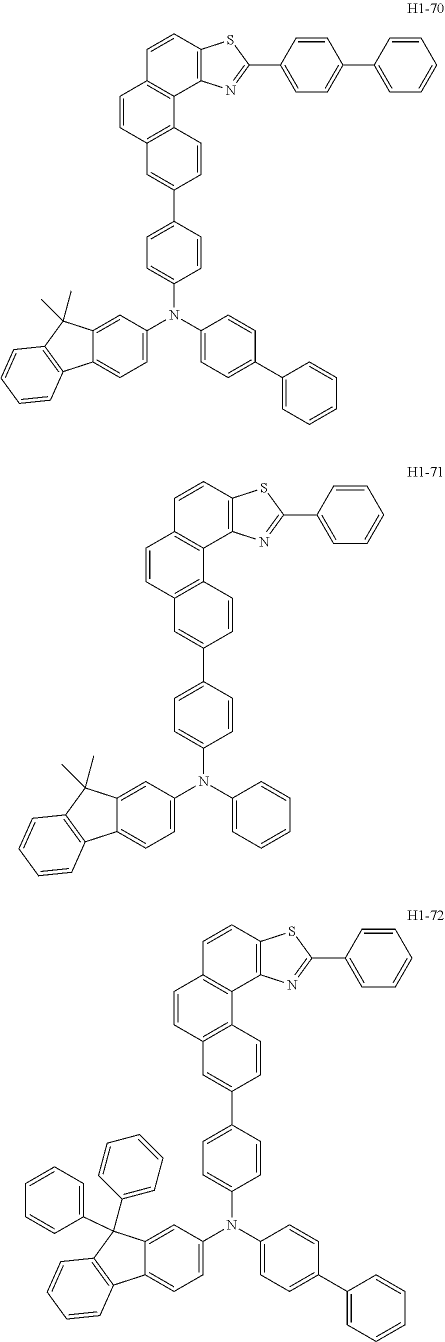 [SMILES: CC1(C)c2ccccc2-c2ccc(N(c3ccc(-c4ccccc4)cc3)c3ccc(-c4ccc5c(ccc6ccc7sc(-c8ccc(-c9ccccc9)cc8)nc7c65)c4)cc3)cc21 ; CC1(C)c2ccccc2-c2ccc(N(c3ccccc3)c3ccc(-c4ccc5c(ccc6ccc7sc(-c8ccccc8)nc7c65)c4)cc3)cc21 ; c1ccc(-c2ccc(N(c3ccc(-c4ccc5c(ccc6ccc7sc(-c8ccccc8)nc7c65)c4)cc3)c3ccc4c(c3)C(c3ccccc3)(c3ccccc3)c3ccccc3-4)cc2)cc1]

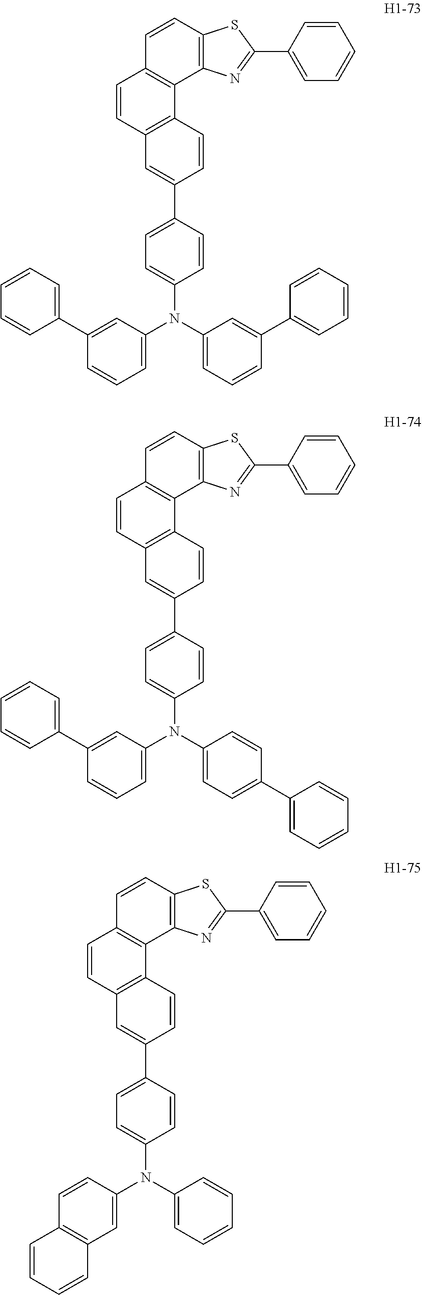

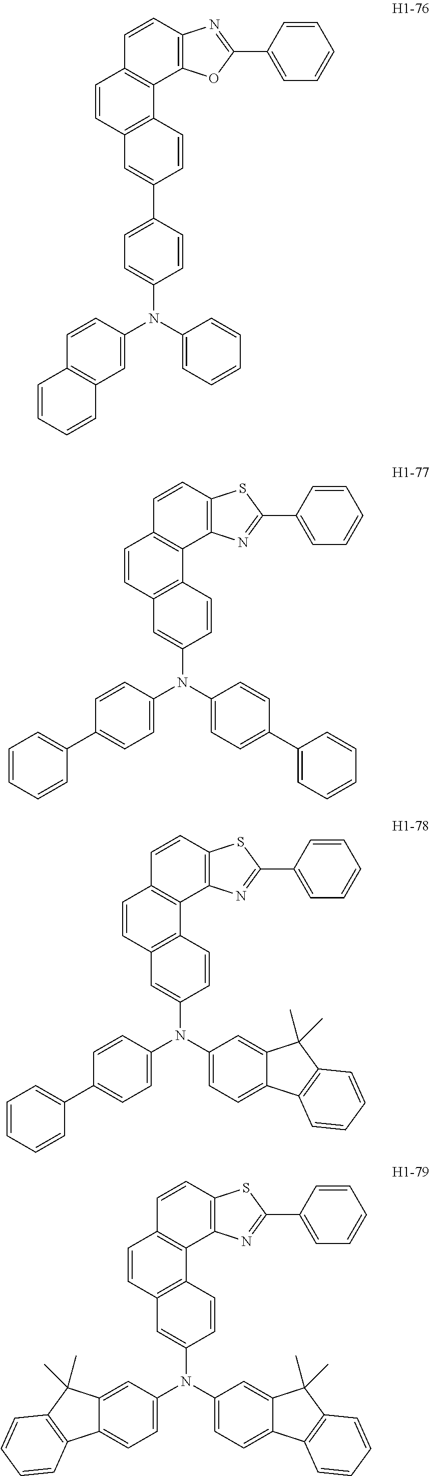

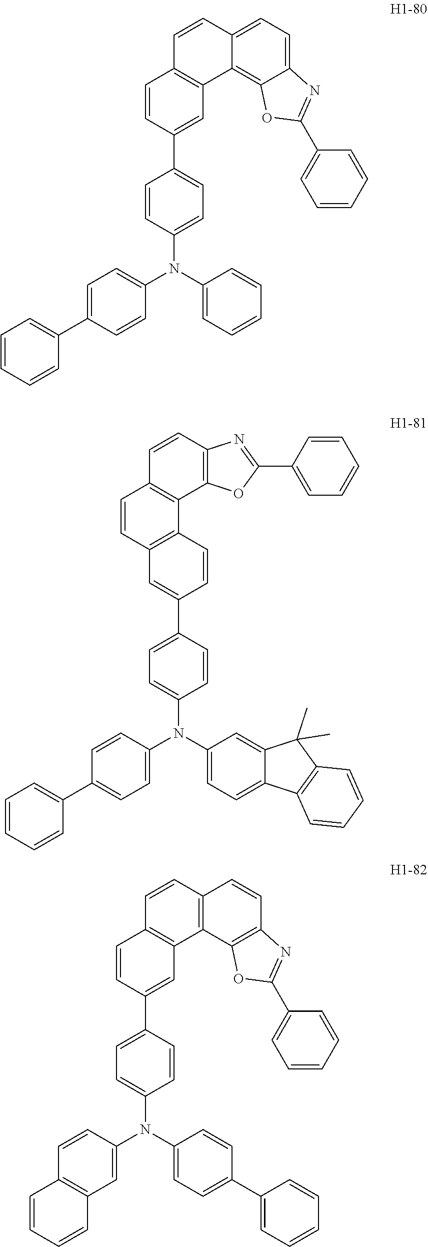 [SMILES: c1ccc(-c2ccc(N(c3ccccc3)c3ccc(-c4ccc5ccc6ccc7nc(-c8ccccc8)oc7c6c5c4)cc3)cc2)cc1 ; CC1(C)c2ccccc2-c2ccc(N(c3ccc(-c4ccccc4)cc3)c3ccc(-c4ccc5c(ccc6ccc7nc(-c8ccccc8)oc7c65)c4)cc3)cc21 ; c1ccc(-c2ccc(N(c3ccc(-c4ccc5ccc6ccc7nc(-c8ccccc8)oc7c6c5c4)cc3)c3ccc4ccccc4c3)cc2)cc1]

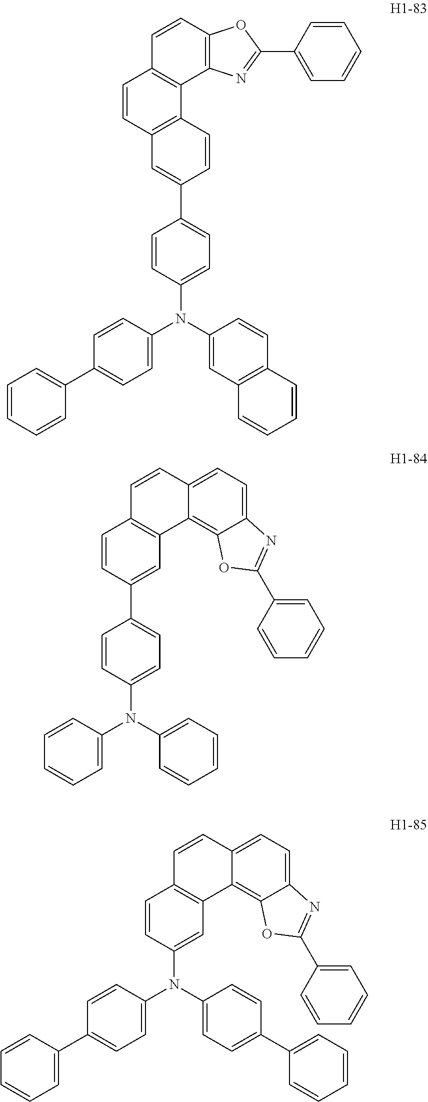 [SMILES: c1ccc(-c2ccc(N(c3ccc(-c4ccc5c(ccc6ccc7oc(-c8ccccc8)nc7c65)c4)cc3)c3ccc4ccccc4c3)cc2)cc1 ; c1ccc(-c2nc3ccc4ccc5ccc(-c6ccc(N(c7ccccc7)c7ccccc7)cc6)cc5c4c3o2)cc1 ; c1ccc(-c2ccc(N(c3ccc(-c4ccccc4)cc3)c3ccc4ccc5ccc6nc(-c7ccccc7)oc6c5c4c3)cc2)cc1]

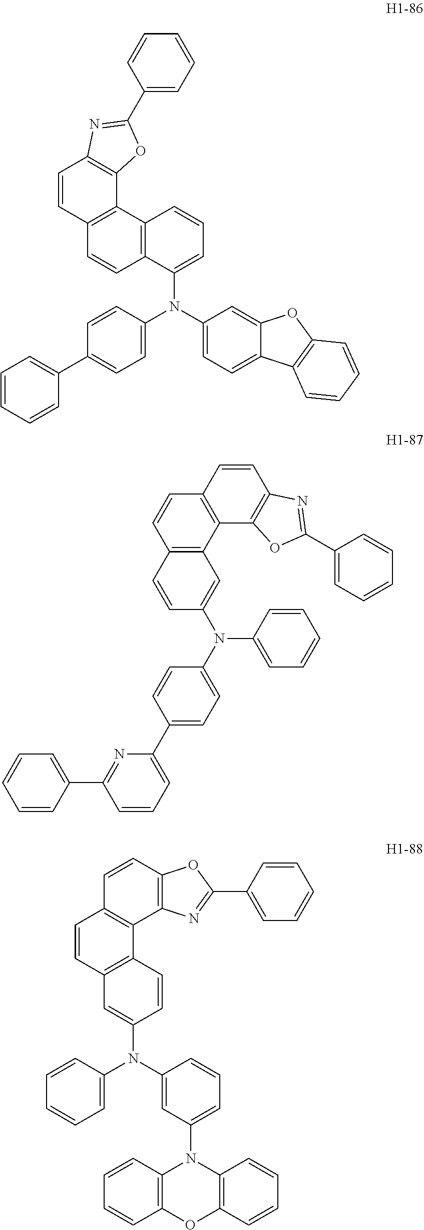 [SMILES: c1ccc(-c2ccc(N(c3ccc4c(c3)oc3ccccc34)c3cccc4c3ccc3ccc5nc(-c6ccccc6)oc5c34)cc2)cc1 ; c1ccc(-c2cccc(-c3ccc(N(c4ccccc4)c4ccc5ccc6ccc7nc(-c8ccccc8)oc7c6c5c4)cc3)n2)cc1 ; c1ccc(-c2nc3c(ccc4ccc5cc(N(c6ccccc6)c6cccc(N7c8ccccc8Oc8ccccc87)c6)ccc5c43)o2)cc1]

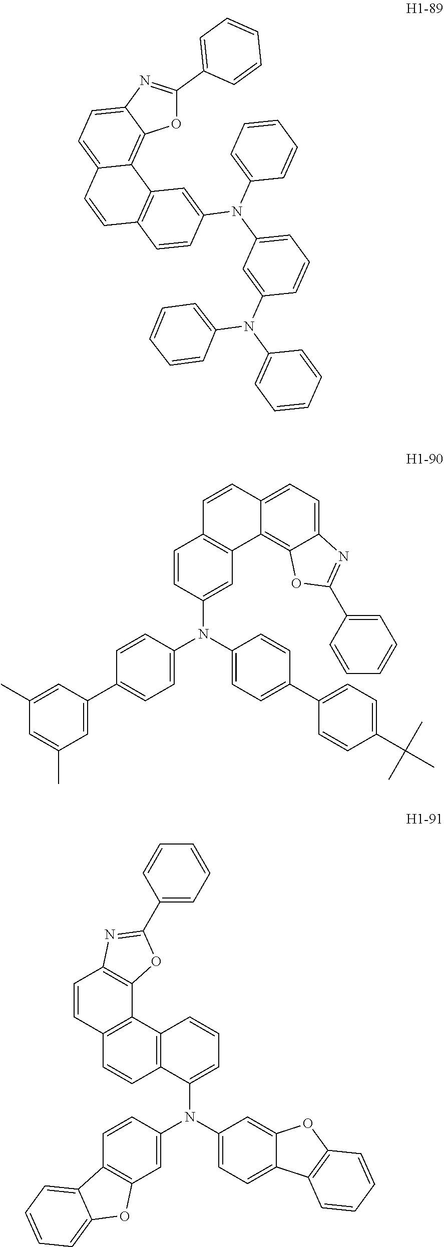 [SMILES: c1ccc(-c2nc3ccc4ccc5ccc(N(c6ccccc6)c6cccc(N(c7ccccc7)c7ccccc7)c6)cc5c4c3o2)cc1 ; Cc1cc(C)cc(-c2ccc(N(c3ccc(-c4ccc(C(C)(C)C)cc4)cc3)c3ccc4ccc5ccc6nc(-c7ccccc7)oc6c5c4c3)cc2)c1 ; c1ccc(-c2nc3ccc4ccc5c(N(c6ccc7c(c6)oc6ccccc67)c6ccc7c(c6)oc6ccccc67)cccc5c4c3o2)cc1]

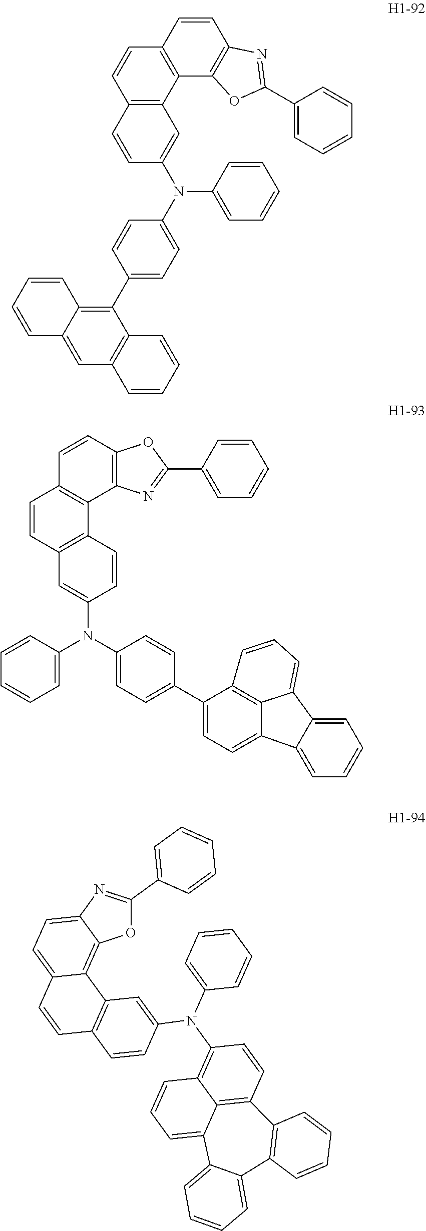 [SMILES: c1ccc(-c2nc3ccc4ccc5ccc(N(c6ccccc6)c6ccc(-c7c8ccccc8cc8ccccc78)cc6)cc5c4c3o2)cc1 ; c1ccc(-c2nc3c(ccc4ccc5cc(N(c6ccccc6)c6ccc(-c7ccc8c9c(cccc79)-c7ccccc7-8)cc6)ccc5c43)o2)cc1 ; c1ccc(-c2nc3ccc4ccc5ccc(N(c6ccccc6)c6ccc7c8c(cccc68)-c6ccccc6-c6ccccc6-7)cc5c4c3o2)cc1]

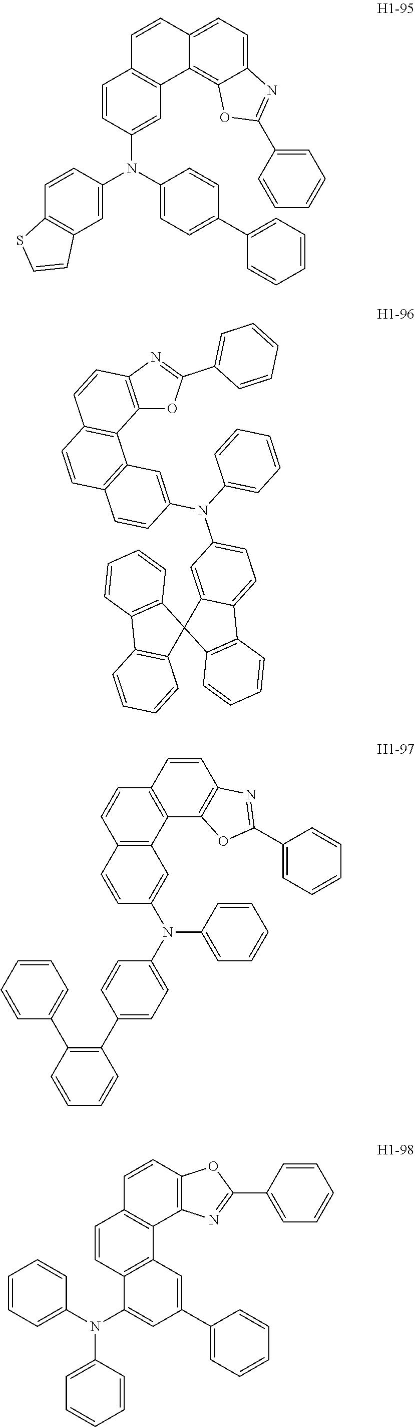

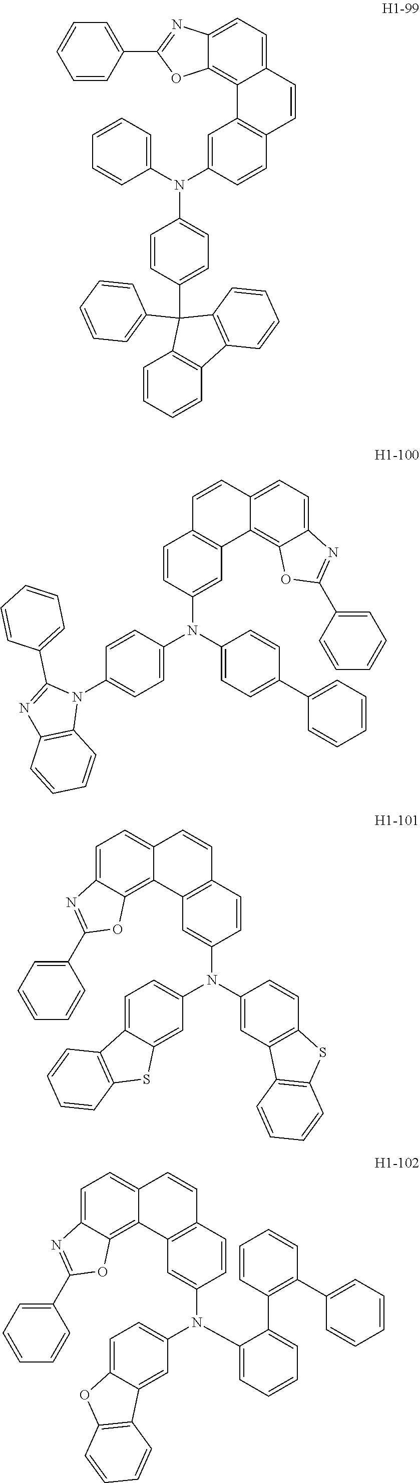 [SMILES: c1ccc(-c2nc3ccc4ccc5ccc(N(c6ccccc6)c6ccc(C7(c8ccccc8)c8ccccc8-c8ccccc87)cc6)cc5c4c3o2)cc1 ; c1ccc(-c2ccc(N(c3ccc(-n4c(-c5ccccc5)nc5ccccc54)cc3)c3ccc4ccc5ccc6nc(-c7ccccc7)oc6c5c4c3)cc2)cc1 ; c1ccc(-c2nc3ccc4ccc5ccc(N(c6ccc7c(c6)sc6ccccc67)c6ccc7sc8ccccc8c7c6)cc5c4c3o2)cc1 ; c1ccc(-c2nc3ccc4ccc5ccc(N(c6ccc7oc8ccccc8c7c6)c6ccccc6-c6ccccc6-c6ccccc6)cc5c4c3o2)cc1]

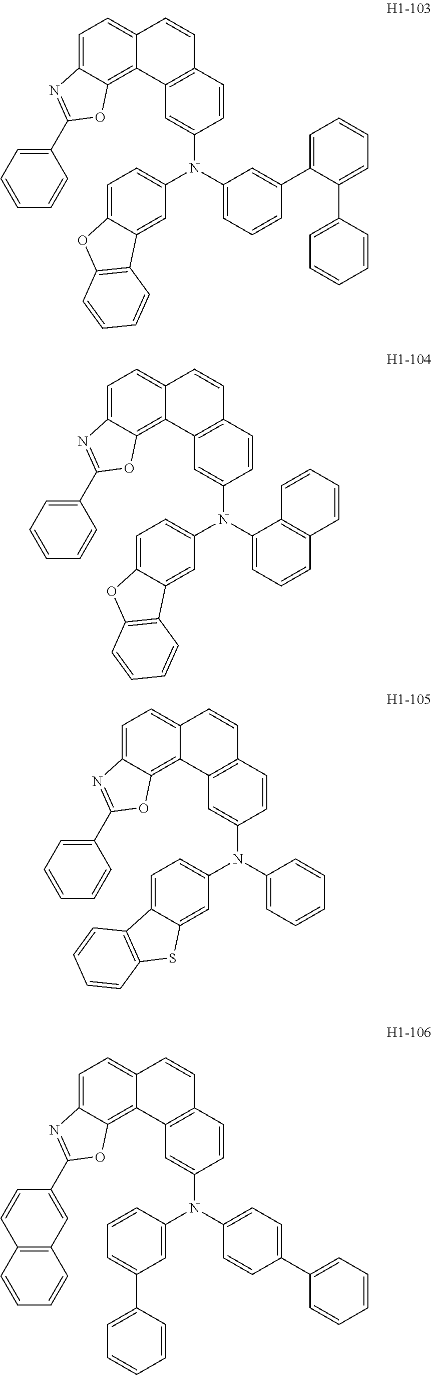

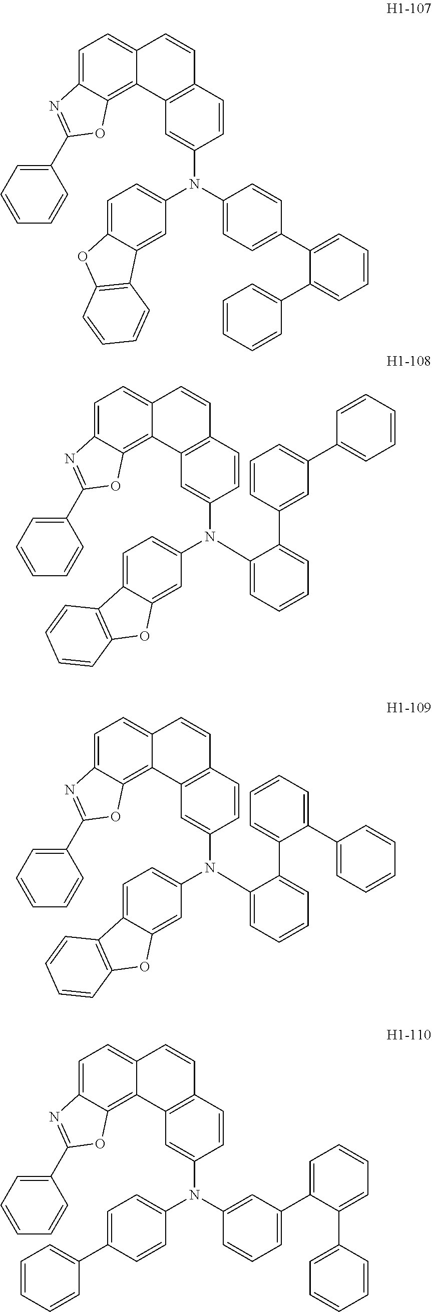 [SMILES: c1ccc(-c2nc3ccc4ccc5ccc(N(c6ccc(-c7ccccc7-c7ccccc7)cc6)c6ccc7oc8ccccc8c7c6)cc5c4c3o2)cc1 ; c1ccc(-c2cccc(-c3ccccc3N(c3ccc4c(c3)oc3ccccc34)c3ccc4ccc5ccc6nc(-c7ccccc7)oc6c5c4c3)c2)cc1 ; c1ccc(-c2nc3ccc4ccc5ccc(N(c6ccc7c(c6)oc6ccccc67)c6ccccc6-c6ccccc6-c6ccccc6)cc5c4c3o2)cc1 ; c1ccc(-c2ccc(N(c3cccc(-c4ccccc4-c4ccccc4)c3)c3ccc4ccc5ccc6nc(-c7ccccc7)oc6c5c4c3)cc2)cc1]

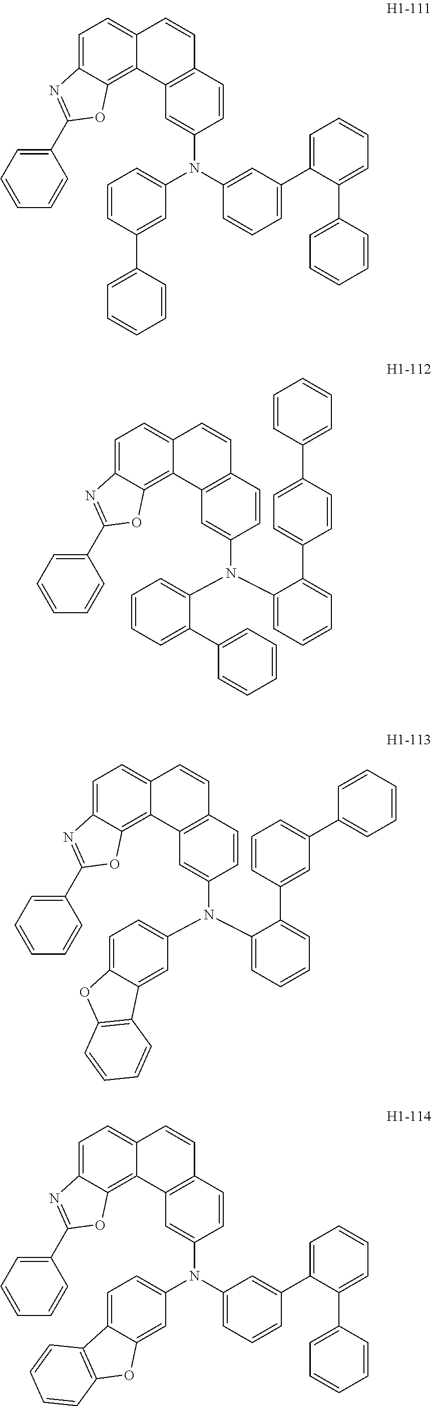

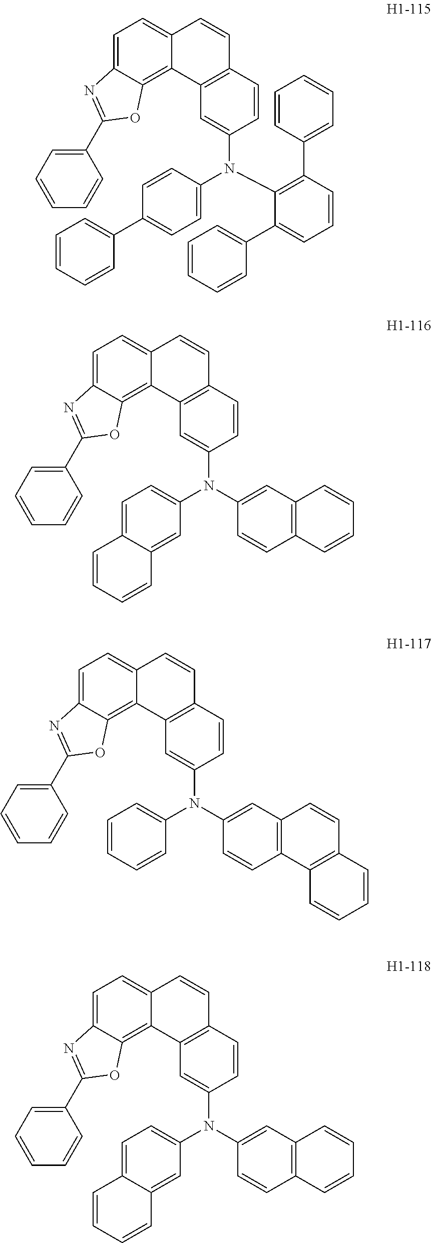 [SMILES: c1ccc(-c2ccc(N(c3ccc4ccc5ccc6nc(-c7ccccc7)oc6c5c4c3)c3c(-c4ccccc4)cccc3-c3ccccc3)cc2)cc1 ; c1ccc(-c2nc3ccc4ccc5ccc(N(c6ccc7ccccc7c6)c6ccc7ccccc7c6)cc5c4c3o2)cc1 ; c1ccc(-c2nc3ccc4ccc5ccc(N(c6ccccc6)c6ccc7c(ccc8ccccc87)c6)cc5c4c3o2)cc1 ; c1ccc(-c2nc3ccc4ccc5ccc(N(c6ccc7ccccc7c6)c6ccc7ccccc7c6)cc5c4c3o2)cc1]

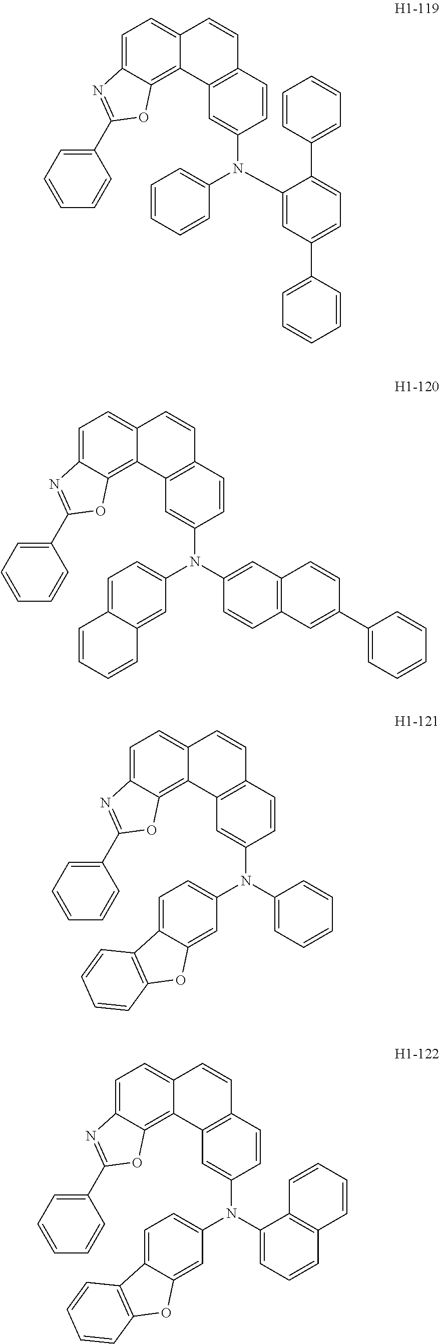 [SMILES: c1ccc(-c2ccc(-c3ccccc3)c(N(c3ccccc3)c3ccc4ccc5ccc6nc(-c7ccccc7)oc6c5c4c3)c2)cc1 ; c1ccc(-c2ccc3cc(N(c4ccc5ccccc5c4)c4ccc5ccc6ccc7nc(-c8ccccc8)oc7c6c5c4)ccc3c2)cc1 ; c1ccc(-c2nc3ccc4ccc5ccc(N(c6ccccc6)c6ccc7c(c6)oc6ccccc67)cc5c4c3o2)cc1 ; c1ccc(-c2nc3ccc4ccc5ccc(N(c6ccc7c(c6)oc6ccccc67)c6cccc7ccccc67)cc5c4c3o2)cc1]

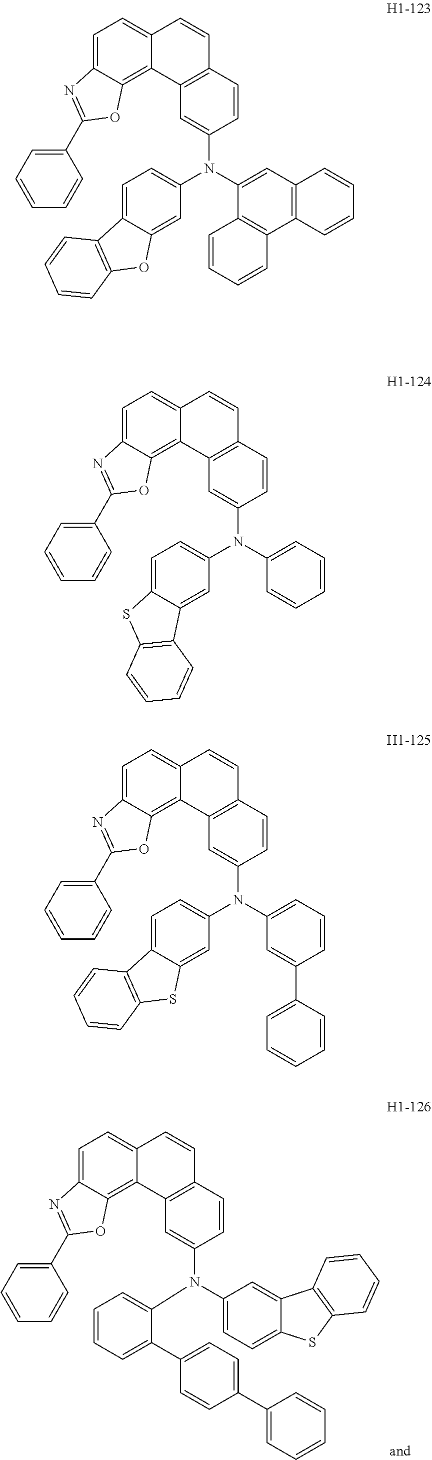 [SMILES: c1ccc(-c2nc3ccc4ccc5ccc(N(c6ccc7c(c6)oc6ccccc67)c6cc7ccccc7c7ccccc67)cc5c4c3o2)cc1 ; c1ccc(-c2nc3ccc4ccc5ccc(N(c6ccccc6)c6ccc7sc8ccccc8c7c6)cc5c4c3o2)cc1 ; c1ccc(-c2cccc(N(c3ccc4c(c3)sc3ccccc34)c3ccc4ccc5ccc6nc(-c7ccccc7)oc6c5c4c3)c2)cc1 ; c1ccc(-c2ccc(-c3ccccc3N(c3ccc4sc5ccccc5c4c3)c3ccc4ccc5ccc6nc(-c7ccccc7)oc6c5c4c3)cc2)cc1]

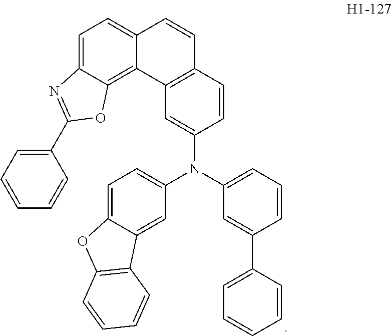 [SMILES: c1ccc(-c2cccc(N(c3ccc4oc5ccccc5c4c3)c3ccc4ccc5ccc6nc(-c7ccccc7)oc6c5c4c3)c2)cc1]

8. The plurality of host materials according to claim 1, wherein the compound represented by formula 2 is at least one selected from the following compounds:

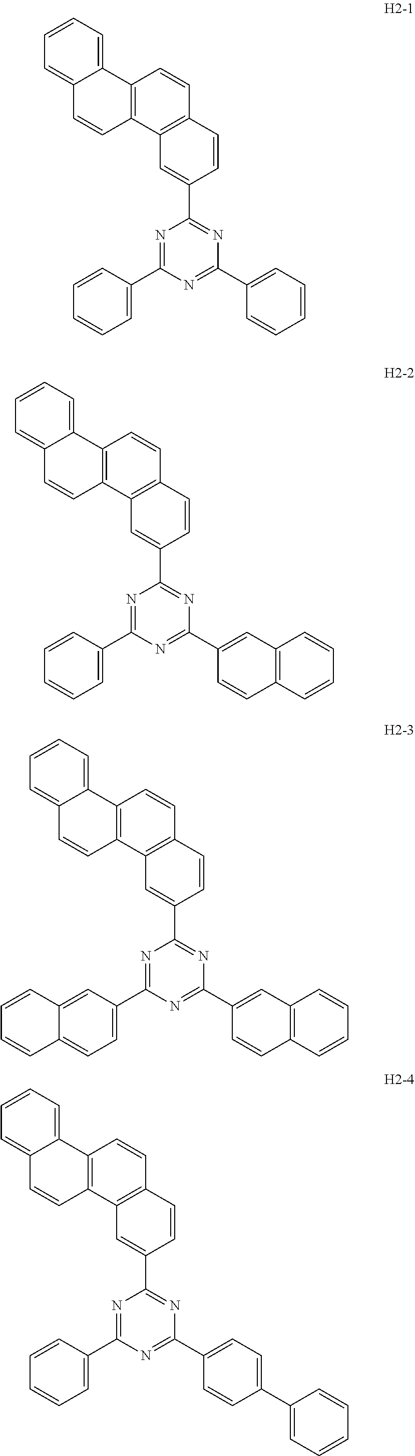

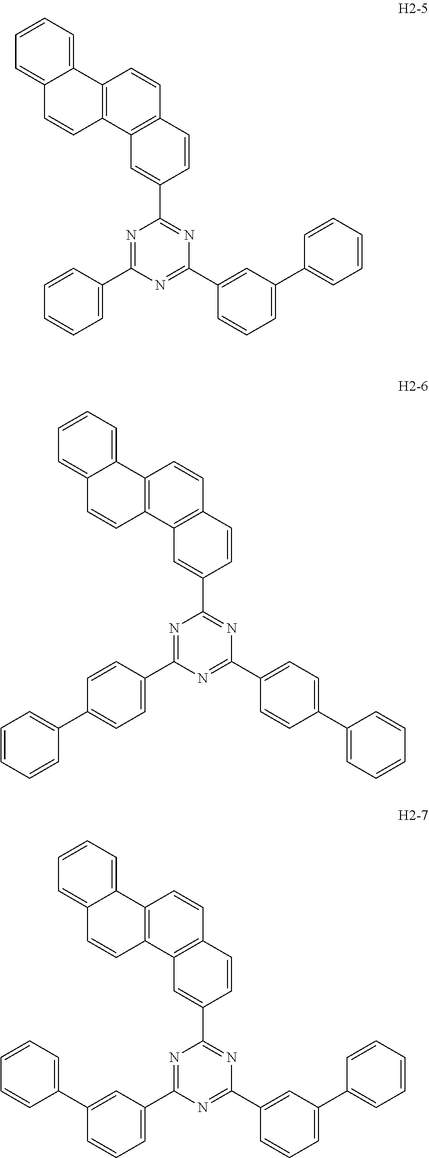 [SMILES: c1ccc(-c2cccc(-c3nc(-c4ccccc4)nc(-c4ccc5ccc6c7ccccc7ccc6c5c4)n3)c2)cc1 ; c1ccc(-c2ccc(-c3nc(-c4ccc(-c5ccccc5)cc4)nc(-c4ccc5ccc6c7ccccc7ccc6c5c4)n3)cc2)cc1 ; c1ccc(-c2cccc(-c3nc(-c4cccc(-c5ccccc5)c4)nc(-c4ccc5ccc6c7ccccc7ccc6c5c4)n3)c2)cc1]

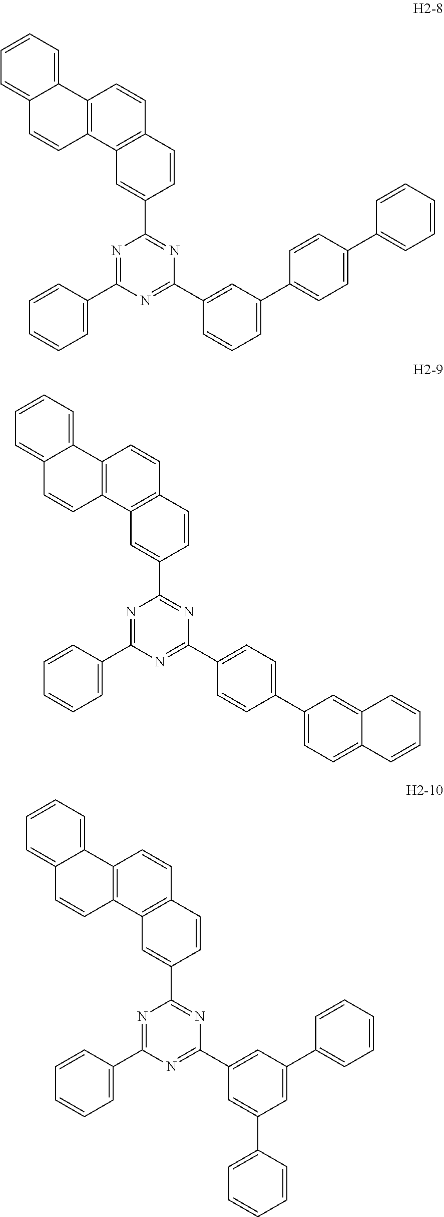

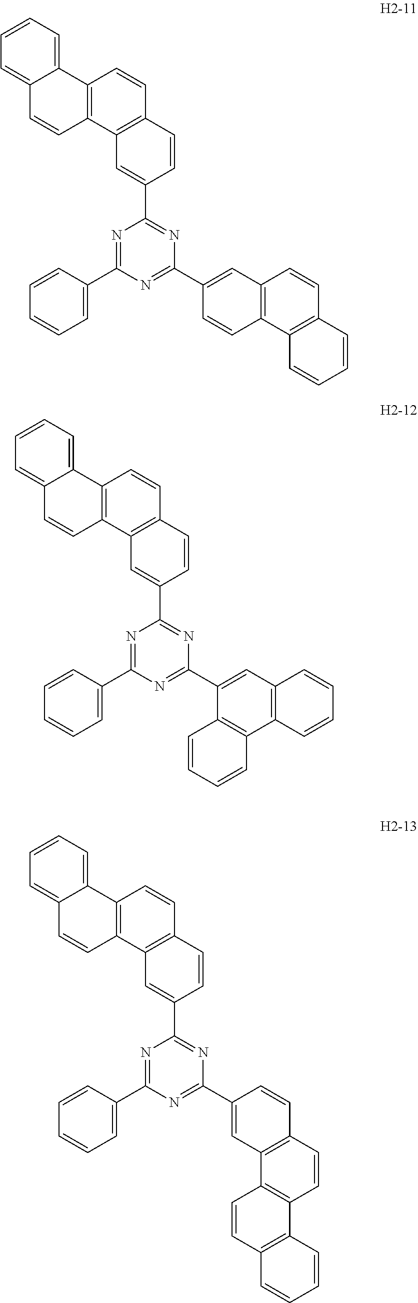 [SMILES: c1ccc(-c2nc(-c3ccc4c(ccc5ccccc54)c3)nc(-c3ccc4ccc5c6ccccc6ccc5c4c3)n2)cc1 ; c1ccc(-c2nc(-c3ccc4ccc5c6ccccc6ccc5c4c3)nc(-c3cc4ccccc4c4ccccc34)n2)cc1 ; c1ccc(-c2nc(-c3ccc4ccc5c6ccccc6ccc5c4c3)nc(-c3ccc4ccc5c6ccccc6ccc5c4c3)n2)cc1]

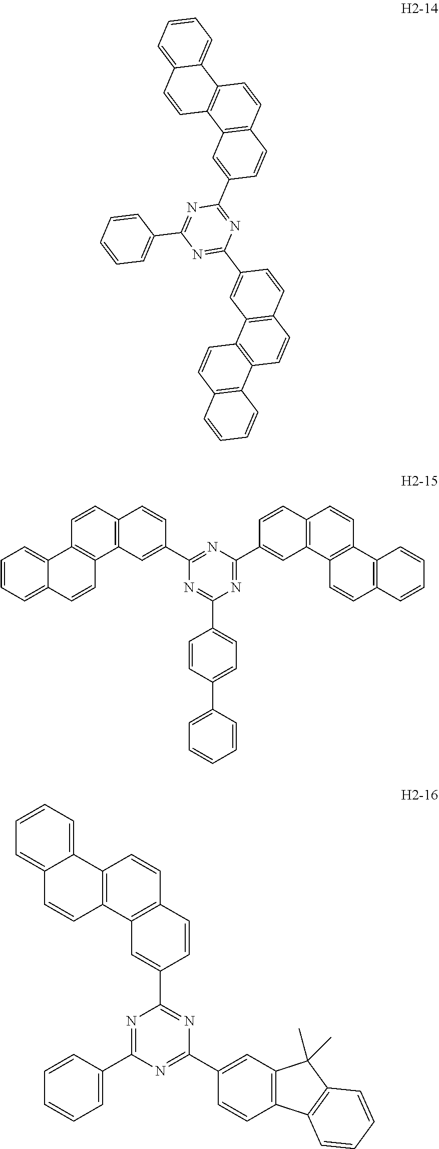 [SMILES: c1ccc(-c2nc(-c3ccc4ccc5c6ccccc6ccc5c4c3)nc(-c3ccc4ccc5c6ccccc6ccc5c4c3)n2)cc1 ; c1ccc(-c2ccc(-c3nc(-c4ccc5ccc6c7ccccc7ccc6c5c4)nc(-c4ccc5ccc6c7ccccc7ccc6c5c4)n3)cc2)cc1 ; CC1(C)c2ccccc2-c2ccc(-c3nc(-c4ccccc4)nc(-c4ccc5ccc6c7ccccc7ccc6c5c4)n3)cc21]

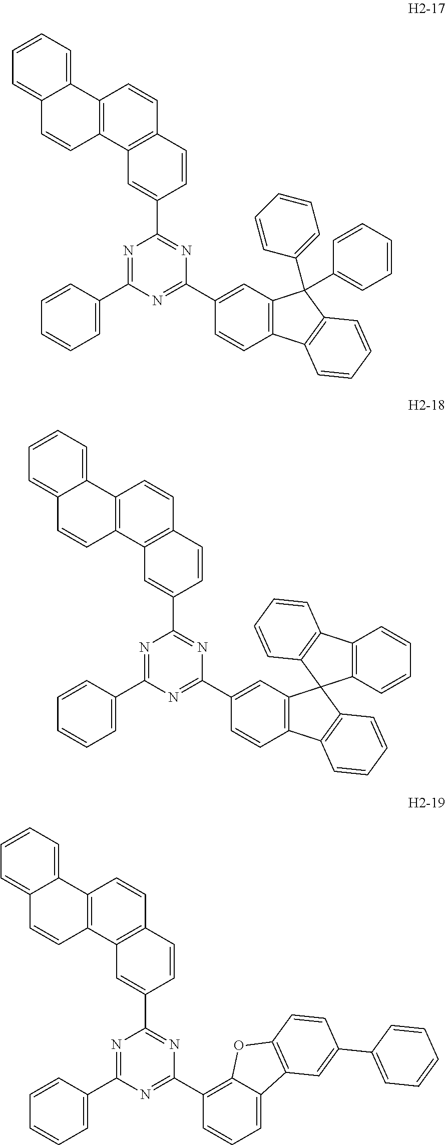 [SMILES: c1ccc(-c2nc(-c3ccc4c(c3)C(c3ccccc3)(c3ccccc3)c3ccccc3-4)nc(-c3ccc4ccc5c6ccccc6ccc5c4c3)n2)cc1 ; c1ccc(-c2nc(-c3ccc4c(c3)C3(c5ccccc5-c5ccccc53)c3ccccc3-4)nc(-c3ccc4ccc5c6ccccc6ccc5c4c3)n2)cc1 ; c1ccc(-c2ccc3oc4c(-c5nc(-c6ccccc6)nc(-c6ccc7ccc8c9ccccc9ccc8c7c6)n5)cccc4c3c2)cc1]

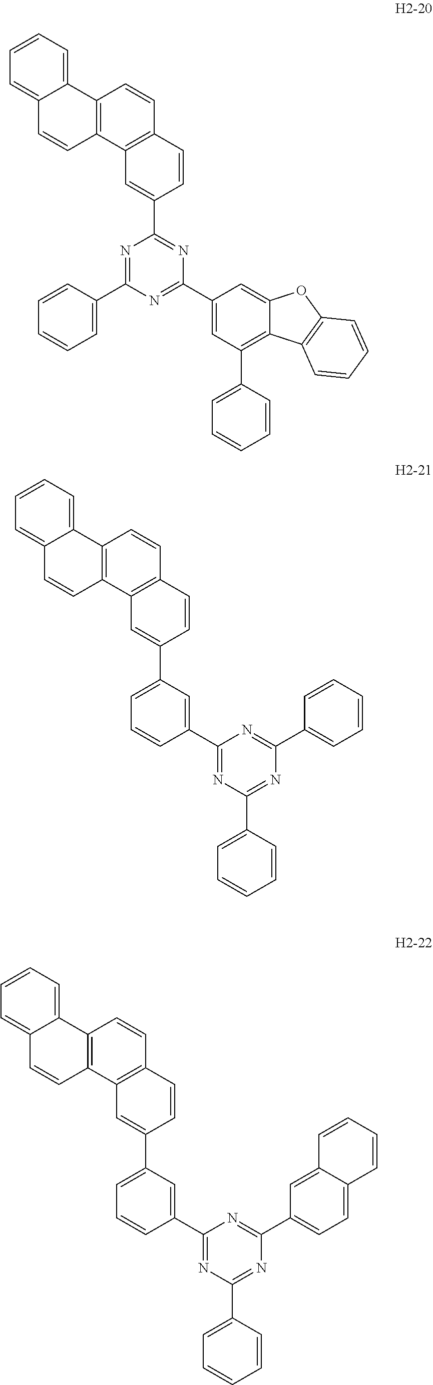 [SMILES: c1ccc(-c2nc(-c3cc(-c4ccccc4)c4c(c3)oc3ccccc34)nc(-c3ccc4ccc5c6ccccc6ccc5c4c3)n2)cc1 ; c1ccc(-c2nc(-c3ccccc3)nc(-c3cccc(-c4ccc5ccc6c7ccccc7ccc6c5c4)c3)n2)cc1 ; c1ccc(-c2nc(-c3cccc(-c4ccc5ccc6c7ccccc7ccc6c5c4)c3)nc(-c3ccc4ccccc4c3)n2)cc1]

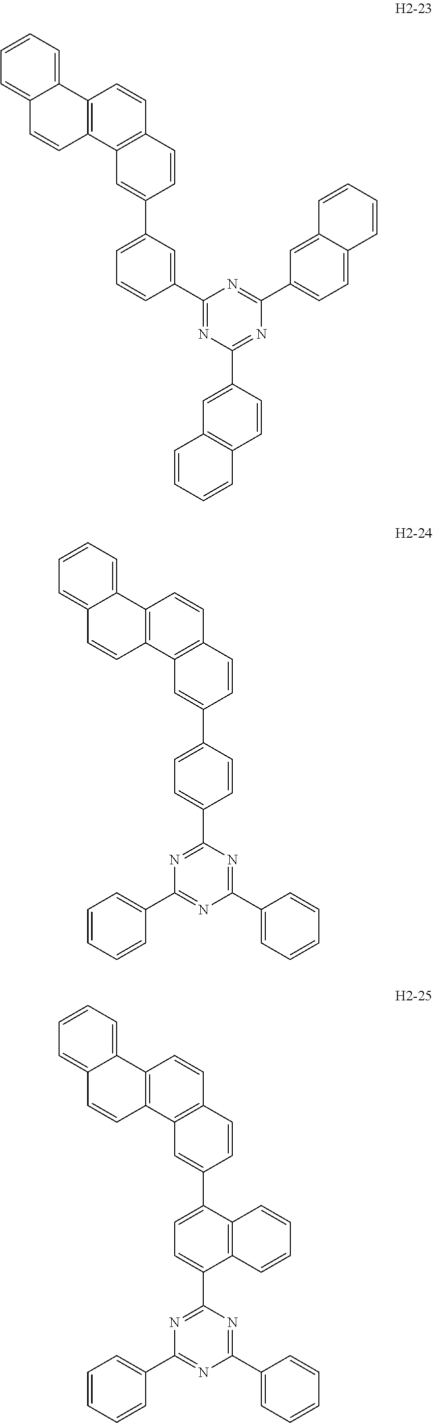 [SMILES: c1cc(-c2ccc3ccc4c5ccccc5ccc4c3c2)cc(-c2nc(-c3ccc4ccccc4c3)nc(-c3ccc4ccccc4c3)n2)c1 ; c1ccc(-c2nc(-c3ccccc3)nc(-c3ccc(-c4ccc5ccc6c7ccccc7ccc6c5c4)cc3)n2)cc1 ; c1ccc(-c2nc(-c3ccccc3)nc(-c3ccc(-c4ccc5ccc6c7ccccc7ccc6c5c4)c4ccccc34)n2)cc1]

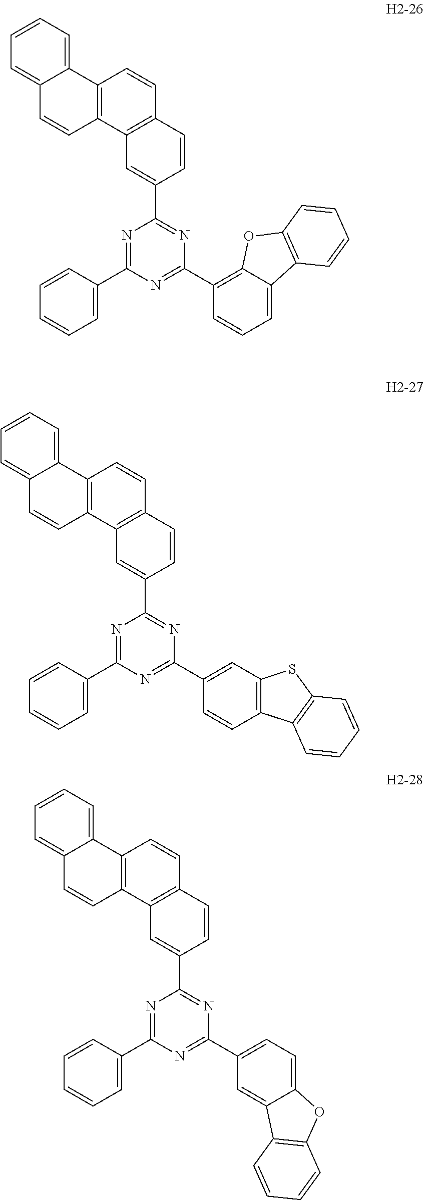 [SMILES: c1ccc(-c2nc(-c3ccc4ccc5c6ccccc6ccc5c4c3)nc(-c3cccc4c3oc3ccccc34)n2)cc1 ; c1ccc(-c2nc(-c3ccc4c(c3)sc3ccccc34)nc(-c3ccc4ccc5c6ccccc6ccc5c4c3)n2)cc1 ; c1ccc(-c2nc(-c3ccc4ccc5c6ccccc6ccc5c4c3)nc(-c3ccc4oc5ccccc5c4c3)n2)cc1]

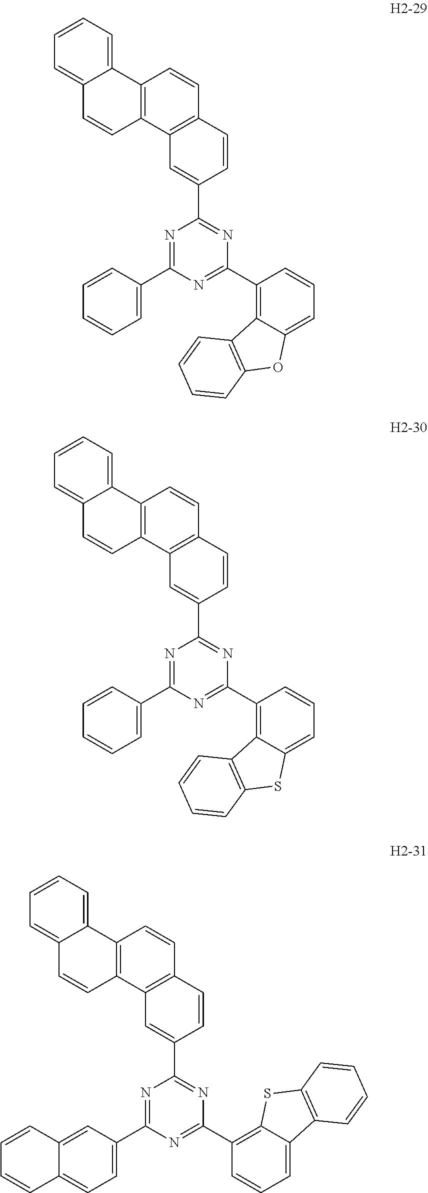 [SMILES: c1ccc(-c2nc(-c3ccc4ccc5c6ccccc6ccc5c4c3)nc(-c3cccc4oc5ccccc5c34)n2)cc1 ; c1ccc(-c2nc(-c3ccc4ccc5c6ccccc6ccc5c4c3)nc(-c3cccc4sc5ccccc5c34)n2)cc1 ; c1ccc2cc(-c3nc(-c4ccc5ccc6c7ccccc7ccc6c5c4)nc(-c4cccc5c4sc4ccccc45)n3)ccc2c1]

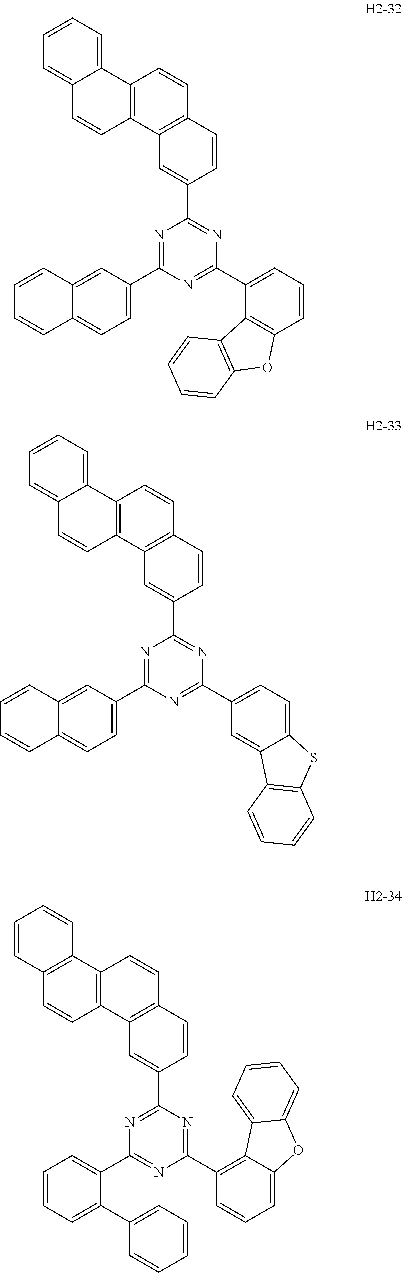 [SMILES: c1ccc2cc(-c3nc(-c4ccc5ccc6c7ccccc7ccc6c5c4)nc(-c4cccc5oc6ccccc6c45)n3)ccc2c1 ; c1ccc2cc(-c3nc(-c4ccc5ccc6c7ccccc7ccc6c5c4)nc(-c4ccc5sc6ccccc6c5c4)n3)ccc2c1 ; c1ccc(-c2ccccc2-c2nc(-c3ccc4ccc5c6ccccc6ccc5c4c3)nc(-c3cccc4oc5ccccc5c34)n2)cc1]

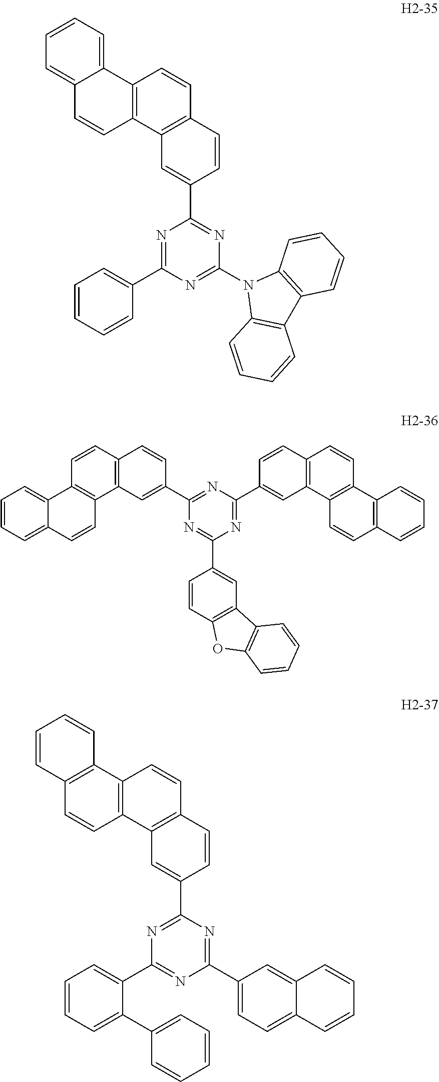 [SMILES: c1ccc(-c2nc(-c3ccc4ccc5c6ccccc6ccc5c4c3)nc(-n3c4ccccc4c4ccccc43)n2)cc1 ; c1ccc2c(c1)ccc1c3cc(-c4nc(-c5ccc6ccc7c8ccccc8ccc7c6c5)nc(-c5ccc6oc7ccccc7c6c5)n4)ccc3ccc21 ; c1ccc(-c2ccccc2-c2nc(-c3ccc4ccccc4c3)nc(-c3ccc4ccc5c6ccccc6ccc5c4c3)n2)cc1]

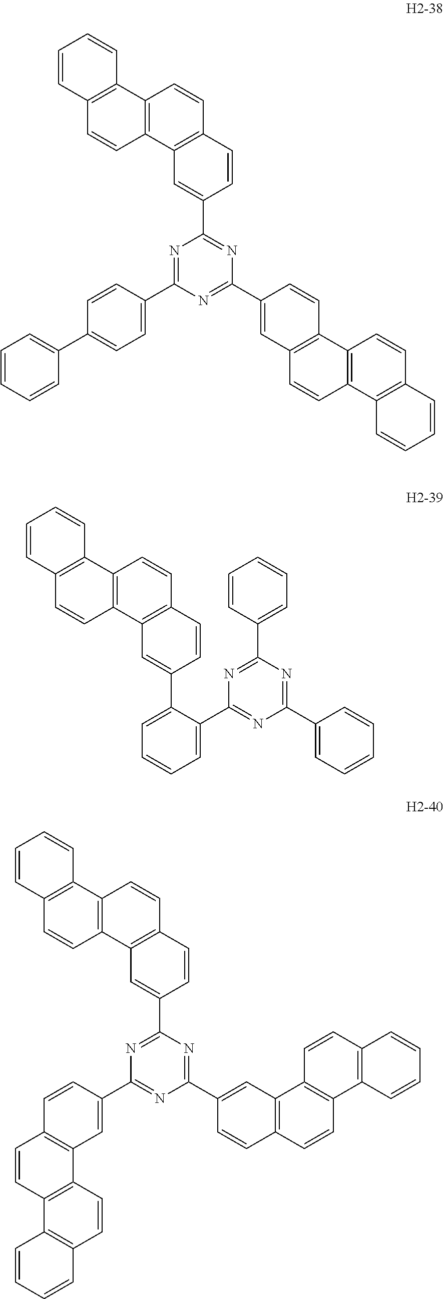 [SMILES: c1ccc(-c2ccc(-c3nc(-c4ccc5c(ccc6c7ccccc7ccc56)c4)nc(-c4ccc5ccc6c7ccccc7ccc6c5c4)n3)cc2)cc1 ; c1ccc(-c2nc(-c3ccccc3)nc(-c3ccccc3-c3ccc4ccc5c6ccccc6ccc5c4c3)n2)cc1 ; c1ccc2c(c1)ccc1c3cc(-c4nc(-c5ccc6ccc7c8ccccc8ccc7c6c5)nc(-c5ccc6ccc7c8ccccc8ccc7c6c5)n4)ccc3ccc21]

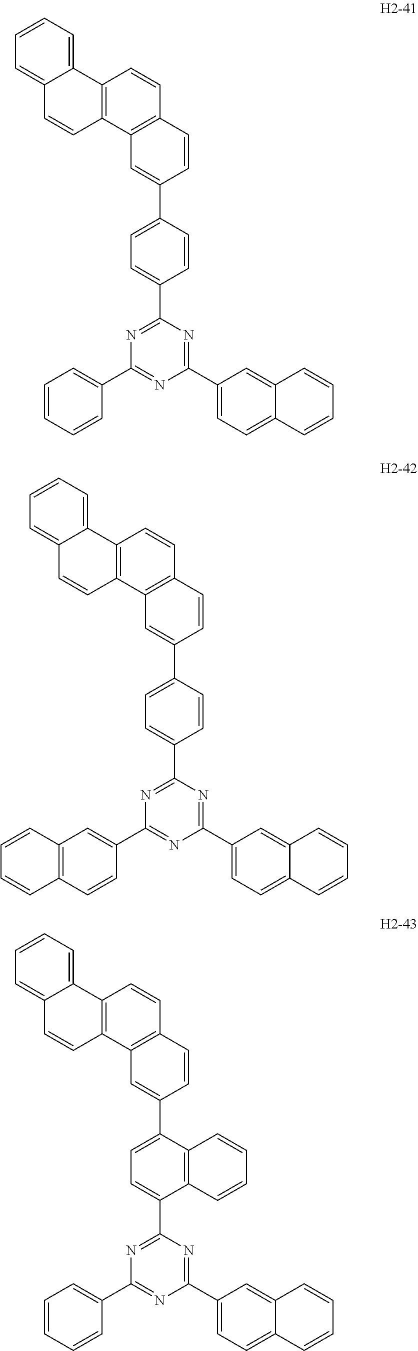

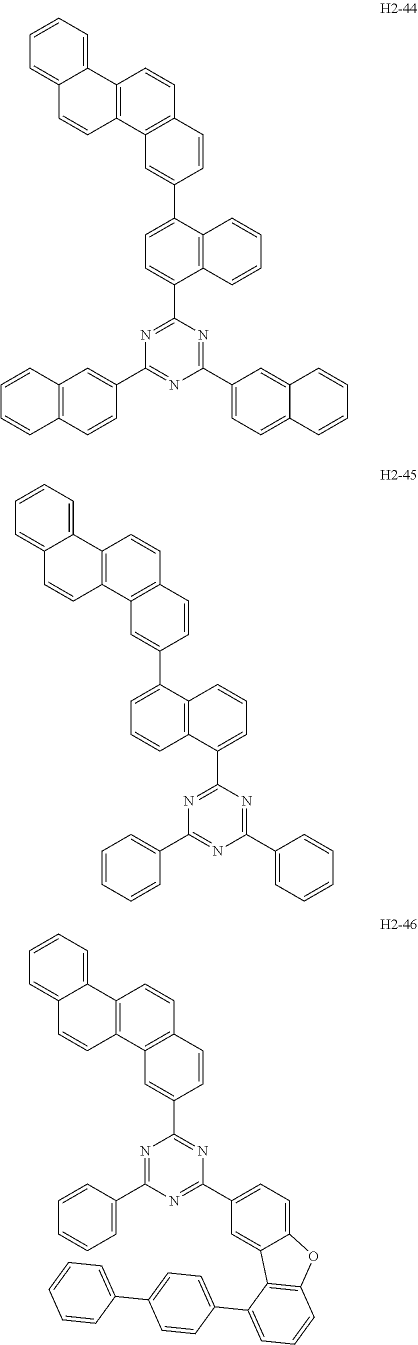 [SMILES: c1ccc2cc(-c3nc(-c4ccc5ccccc5c4)nc(-c4ccc(-c5ccc6ccc7c8ccccc8ccc7c6c5)c5ccccc45)n3)ccc2c1 ; c1ccc(-c2nc(-c3ccccc3)nc(-c3cccc4c(-c5ccc6ccc7c8ccccc8ccc7c6c5)cccc34)n2)cc1 ; c1ccc(-c2ccc(-c3cccc4oc5ccc(-c6nc(-c7ccccc7)nc(-c7ccc8ccc9c%10ccccc%10ccc9c8c7)n6)cc5c34)cc2)cc1]

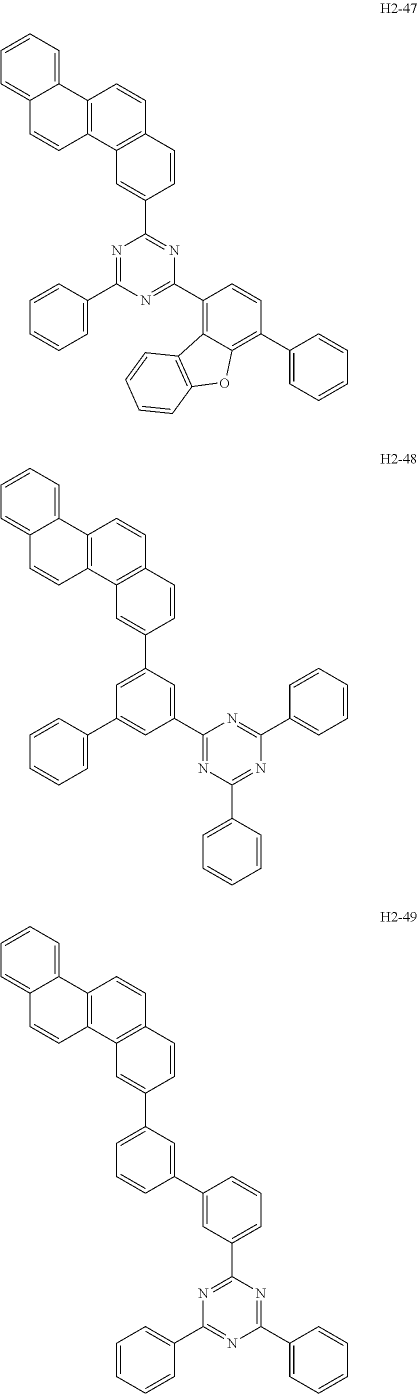 [SMILES: c1ccc(-c2nc(-c3ccc4ccc5c6ccccc6ccc5c4c3)nc(-c3ccc(-c4ccccc4)c4oc5ccccc5c34)n2)cc1 ; c1ccc(-c2cc(-c3ccc4ccc5c6ccccc6ccc5c4c3)cc(-c3nc(-c4ccccc4)nc(-c4ccccc4)n3)c2)cc1 ; c1ccc(-c2nc(-c3ccccc3)nc(-c3cccc(-c4cccc(-c5ccc6ccc7c8ccccc8ccc7c6c5)c4)c3)n2)cc1]

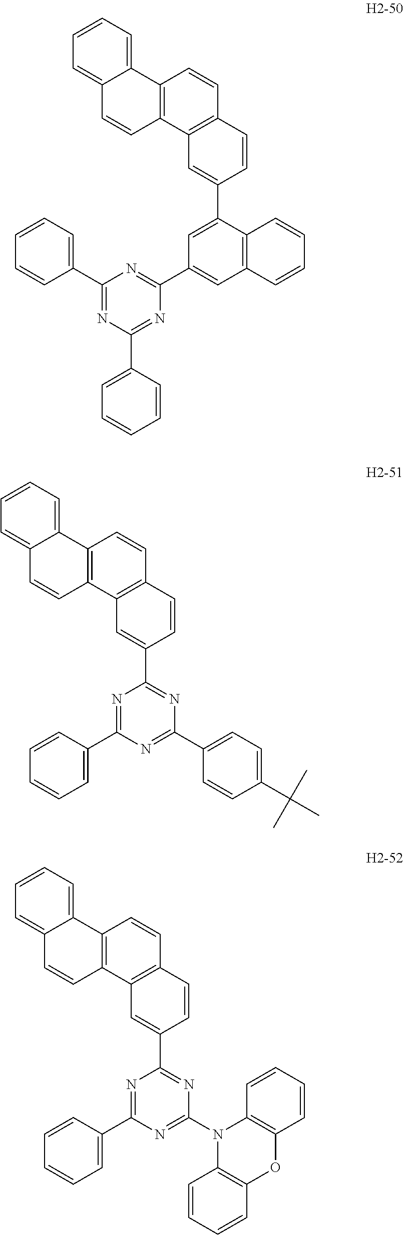 [SMILES: c1ccc(-c2nc(-c3ccccc3)nc(-c3cc(-c4ccc5ccc6c7ccccc7ccc6c5c4)c4ccccc4c3)n2)cc1 ; CC(C)(C)c1ccc(-c2nc(-c3ccccc3)nc(-c3ccc4ccc5c6ccccc6ccc5c4c3)n2)cc1 ; c1ccc(-c2nc(-c3ccc4ccc5c6ccccc6ccc5c4c3)nc(N3c4ccccc4Oc4ccccc43)n2)cc1]

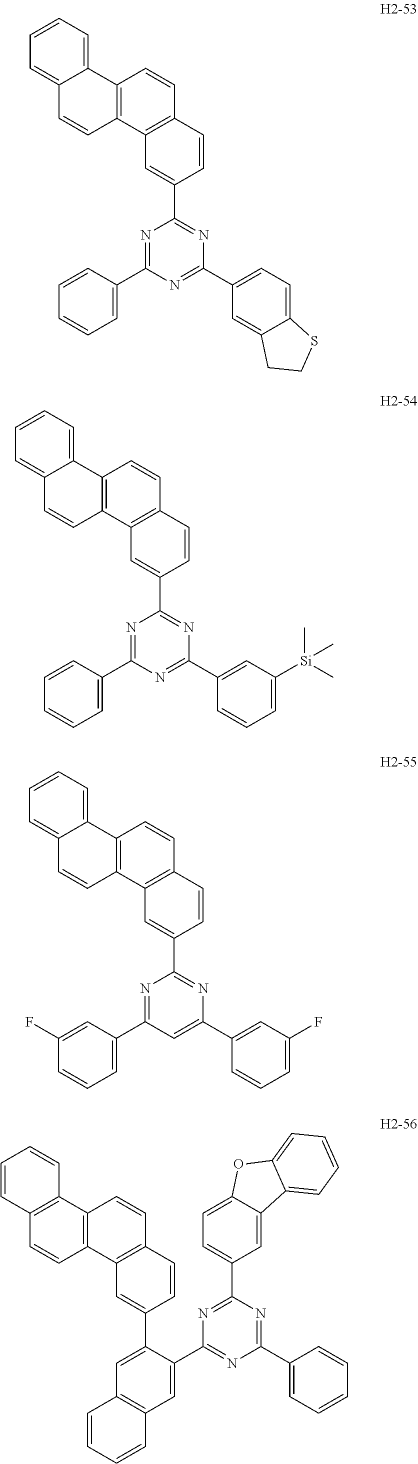 [SMILES: c1ccc(-c2nc(-c3ccc4c(c3)CCS4)nc(-c3ccc4ccc5c6ccccc6ccc5c4c3)n2)cc1 ; C[Si](C)(C)c1cccc(-c2nc(-c3ccccc3)nc(-c3ccc4ccc5c6ccccc6ccc5c4c3)n2)c1 ; Fc1cccc(-c2cc(-c3cccc(F)c3)nc(-c3ccc4ccc5c6ccccc6ccc5c4c3)n2)c1 ; c1ccc(-c2nc(-c3ccc4oc5ccccc5c4c3)nc(-c3cc4ccccc4cc3-c3ccc4ccc5c6ccccc6ccc5c4c3)n2)cc1]

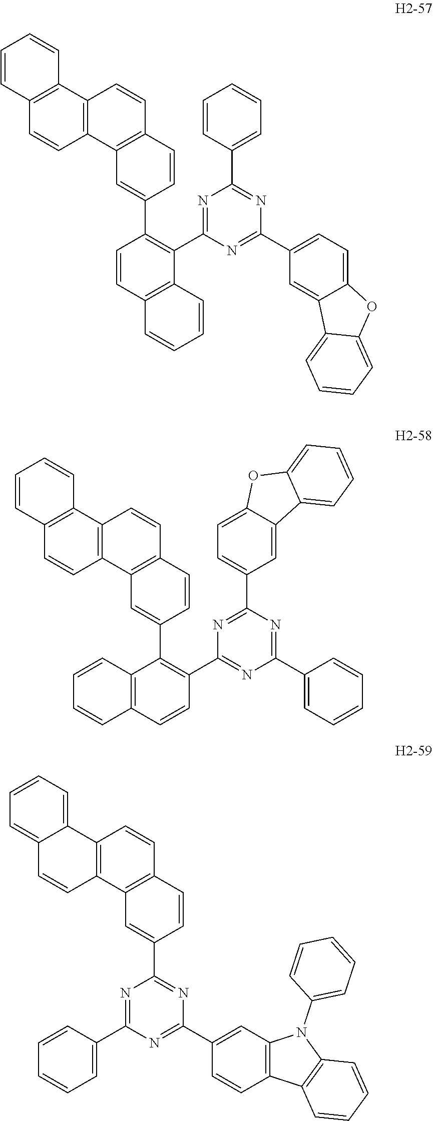 [SMILES: c1ccc(-c2nc(-c3ccc4oc5ccccc5c4c3)nc(-c3c(-c4ccc5ccc6c7ccccc7ccc6c5c4)ccc4ccccc34)n2)cc1 ; c1ccc(-c2nc(-c3ccc4oc5ccccc5c4c3)nc(-c3ccc4ccccc4c3-c3ccc4ccc5c6ccccc6ccc5c4c3)n2)cc1 ; c1ccc(-c2nc(-c3ccc4ccc5c6ccccc6ccc5c4c3)nc(-c3ccc4c5ccccc5n(-c5ccccc5)c4c3)n2)cc1]

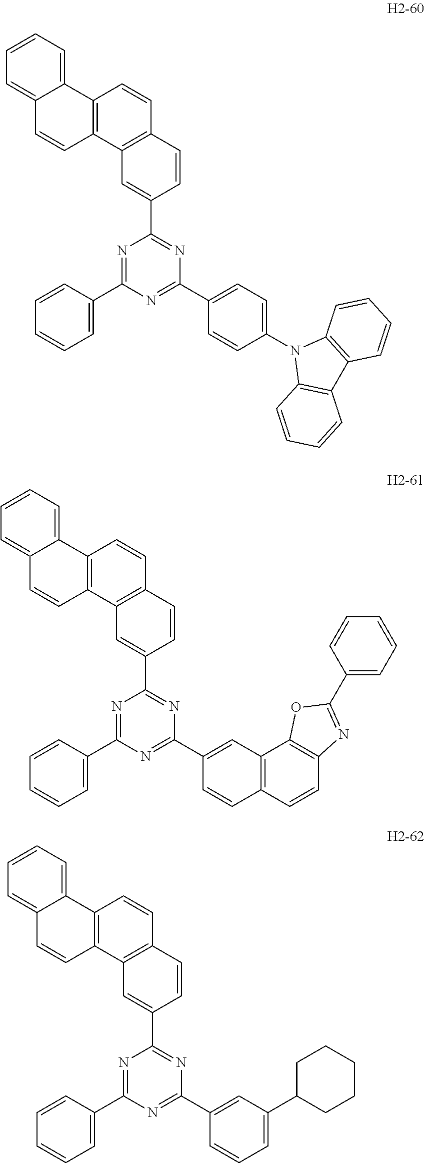

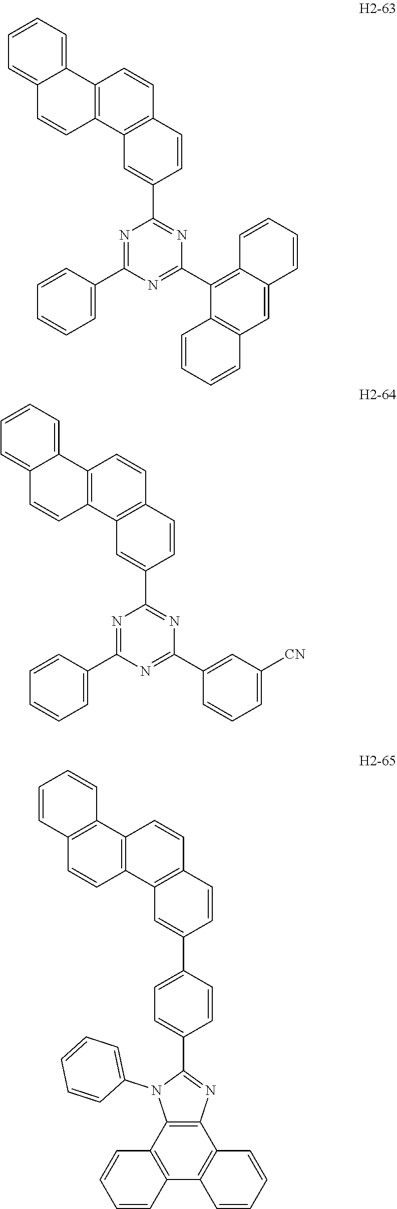 [SMILES: c1ccc(-c2nc(-c3ccc4ccc5c6ccccc6ccc5c4c3)nc(-c3c4ccccc4cc4ccccc34)n2)cc1 ; N#Cc1cccc(-c2nc(-c3ccccc3)nc(-c3ccc4ccc5c6ccccc6ccc5c4c3)n2)c1 ; c1ccc(-n2c(-c3ccc(-c4ccc5ccc6c7ccccc7ccc6c5c4)cc3)nc3c4ccccc4c4ccccc4c32)cc1]

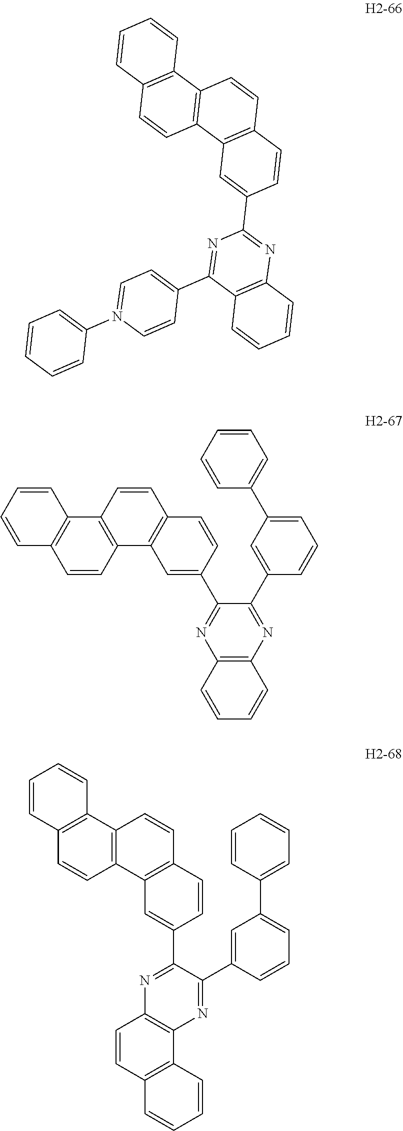

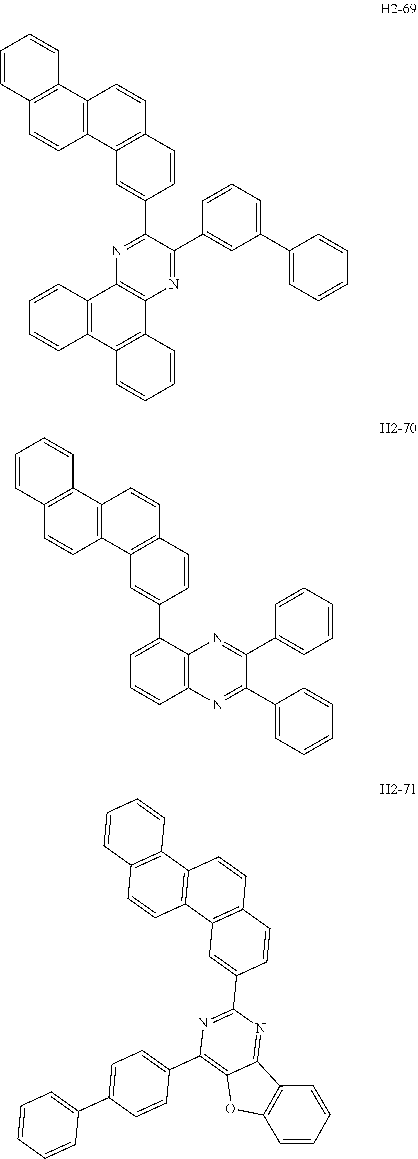 [SMILES: c1ccc(-c2cccc(-c3nc4c5ccccc5c5ccccc5c4nc3-c3ccc4ccc5c6ccccc6ccc5c4c3)c2)cc1 ; c1ccc(-c2nc3cccc(-c4ccc5ccc6c7ccccc7ccc6c5c4)c3nc2-c2ccccc2)cc1 ; c1ccc(-c2ccc(-c3nc(-c4ccc5ccc6c7ccccc7ccc6c5c4)nc4c3oc3ccccc34)cc2)cc1]

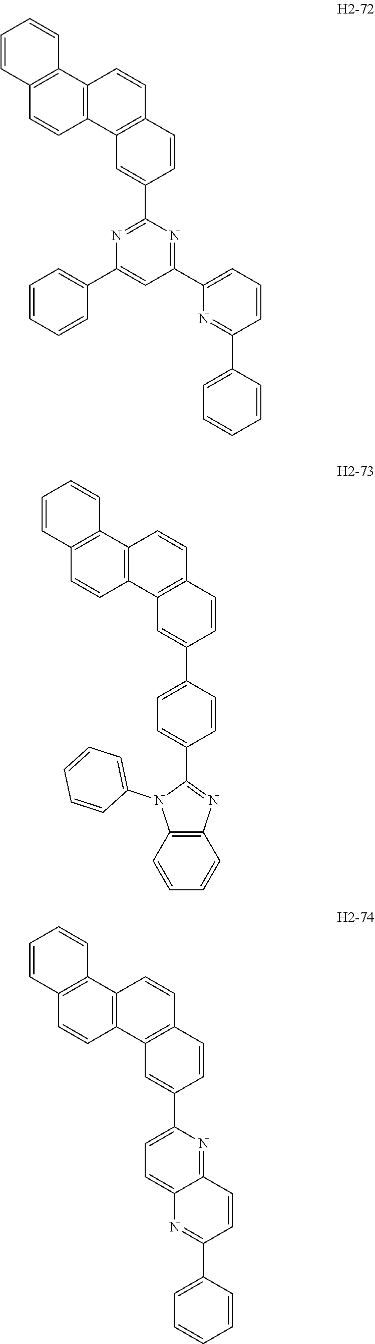 [SMILES: c1ccc(-c2cccc(-c3cc(-c4ccccc4)nc(-c4ccc5ccc6c7ccccc7ccc6c5c4)n3)n2)cc1 ; c1ccc(-n2c(-c3ccc(-c4ccc5ccc6c7ccccc7ccc6c5c4)cc3)nc3ccccc32)cc1 ; c1ccc(-c2ccc3nc(-c4ccc5ccc6c7ccccc7ccc6c5c4)ccc3n2)cc1]

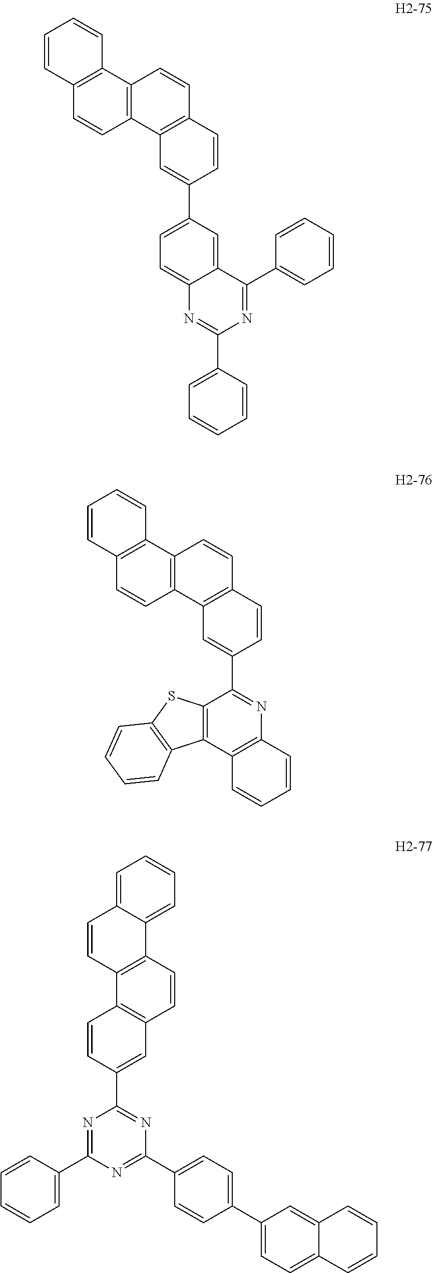 [SMILES: c1ccc(-c2nc(-c3ccccc3)c3cc(-c4ccc5ccc6c7ccccc7ccc6c5c4)ccc3n2)cc1 ; c1ccc2c(c1)ccc1c3cc(-c4nc5ccccc5c5c4sc4ccccc45)ccc3ccc21 ; c1ccc(-c2nc(-c3ccc(-c4ccc5ccccc5c4)cc3)nc(-c3ccc4c(ccc5c6ccccc6ccc45)c3)n2)cc1]

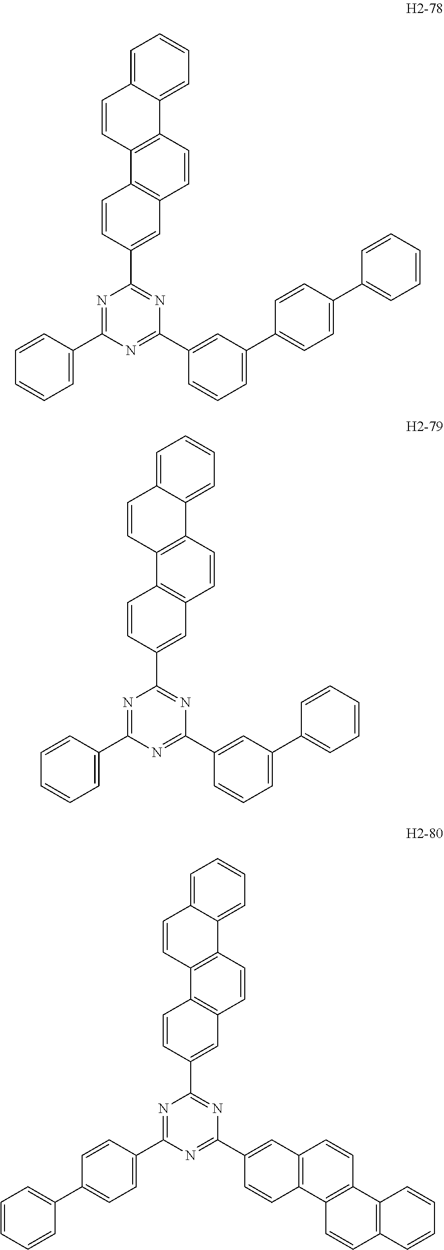 [SMILES: c1ccc(-c2ccc(-c3cccc(-c4nc(-c5ccccc5)nc(-c5ccc6c(ccc7c8ccccc8ccc67)c5)n4)c3)cc2)cc1 ; c1ccc(-c2cccc(-c3nc(-c4ccccc4)nc(-c4ccc5c(ccc6c7ccccc7ccc56)c4)n3)c2)cc1 ; c1ccc(-c2ccc(-c3nc(-c4ccc5c(ccc6c7ccccc7ccc56)c4)nc(-c4ccc5c(ccc6c7ccccc7ccc56)c4)n3)cc2)cc1]

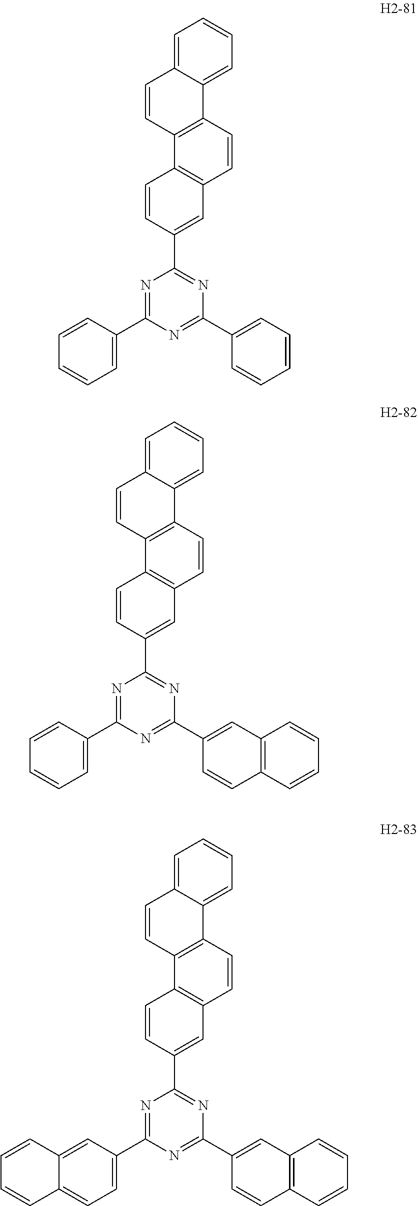 [SMILES: c1ccc(-c2nc(-c3ccccc3)nc(-c3ccc4c(ccc5c6ccccc6ccc45)c3)n2)cc1 ; c1ccc(-c2nc(-c3ccc4ccccc4c3)nc(-c3ccc4c(ccc5c6ccccc6ccc45)c3)n2)cc1 ; c1ccc2cc(-c3nc(-c4ccc5ccccc5c4)nc(-c4ccc5c(ccc6c7ccccc7ccc56)c4)n3)ccc2c1]

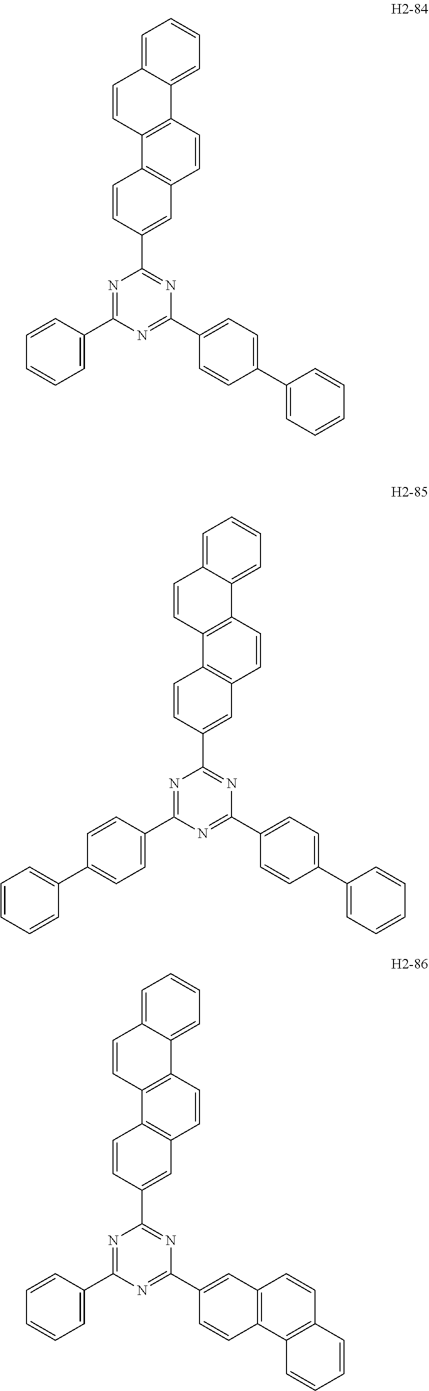 [SMILES: c1ccc(-c2ccc(-c3nc(-c4ccccc4)nc(-c4ccc5c(ccc6c7ccccc7ccc56)c4)n3)cc2)cc1 ; c1ccc(-c2ccc(-c3nc(-c4ccc(-c5ccccc5)cc4)nc(-c4ccc5c(ccc6c7ccccc7ccc56)c4)n3)cc2)cc1 ; c1ccc(-c2nc(-c3ccc4c(ccc5ccccc54)c3)nc(-c3ccc4c(ccc5c6ccccc6ccc45)c3)n2)cc1]

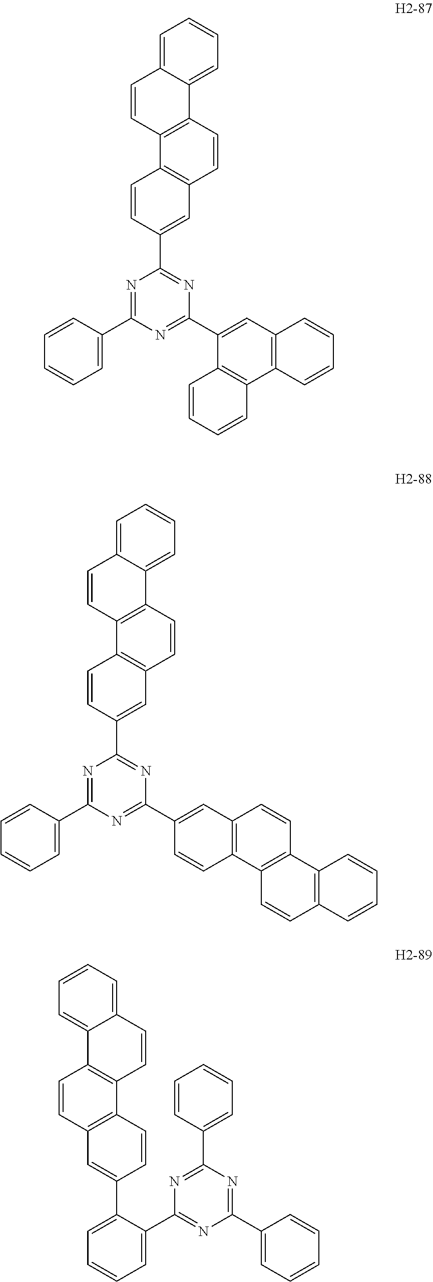 [SMILES: c1ccc(-c2nc(-c3ccc4c(ccc5c6ccccc6ccc45)c3)nc(-c3cc4ccccc4c4ccccc34)n2)cc1 ; c1ccc(-c2nc(-c3ccc4c(ccc5c6ccccc6ccc45)c3)nc(-c3ccc4c(ccc5c6ccccc6ccc45)c3)n2)cc1 ; c1ccc(-c2nc(-c3ccccc3)nc(-c3ccccc3-c3ccc4c(ccc5c6ccccc6ccc45)c3)n2)cc1]

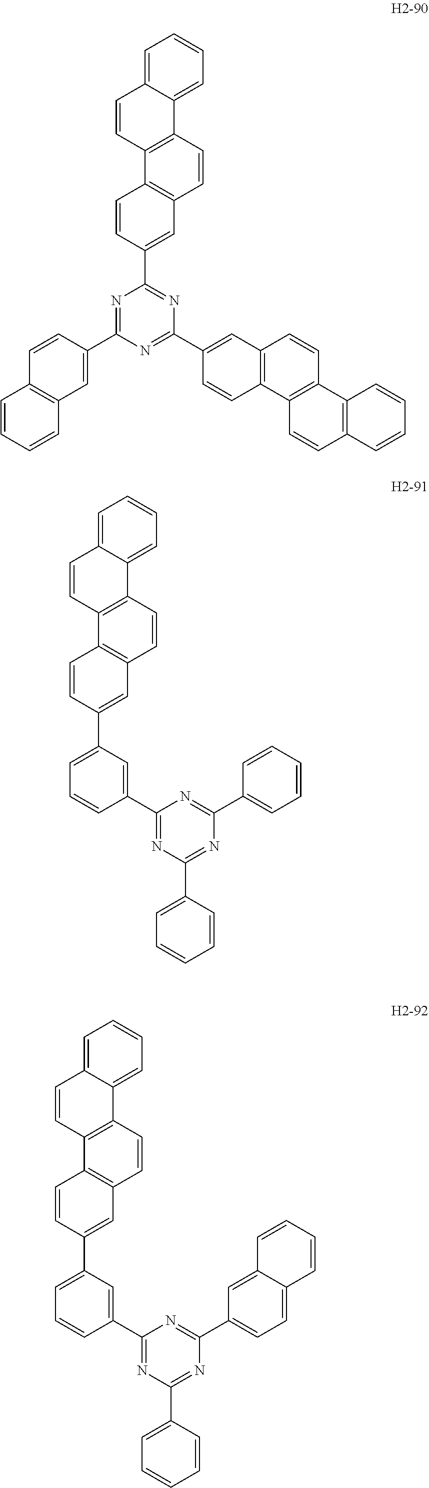 [SMILES: c1ccc2cc(-c3nc(-c4ccc5c(ccc6c7ccccc7ccc56)c4)nc(-c4ccc5c(ccc6c7ccccc7ccc56)c4)n3)ccc2c1 ; c1ccc(-c2nc(-c3ccccc3)nc(-c3cccc(-c4ccc5c(ccc6c7ccccc7ccc56)c4)c3)n2)cc1 ; c1ccc(-c2nc(-c3cccc(-c4ccc5c(ccc6c7ccccc7ccc56)c4)c3)nc(-c3ccc4ccccc4c3)n2)cc1]

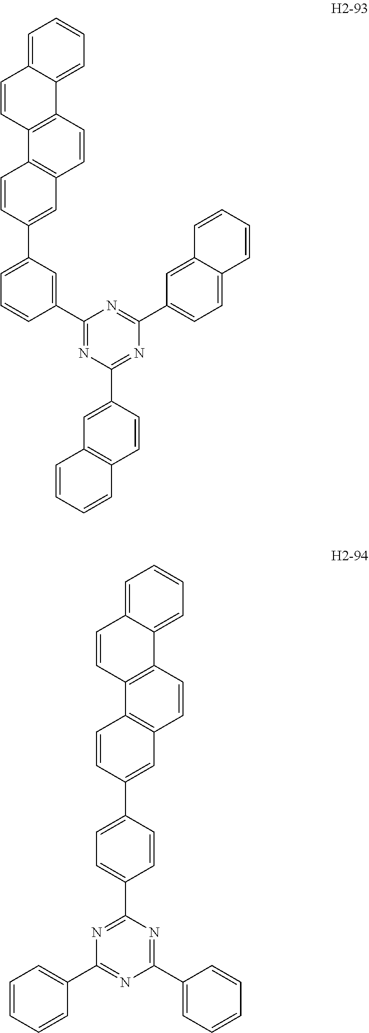 [SMILES: c1cc(-c2ccc3c(ccc4c5ccccc5ccc34)c2)cc(-c2nc(-c3ccc4ccccc4c3)nc(-c3ccc4ccccc4c3)n2)c1 ; c1ccc(-c2nc(-c3ccccc3)nc(-c3ccc(-c4ccc5c(ccc6c7ccccc7ccc56)c4)cc3)n2)cc1]

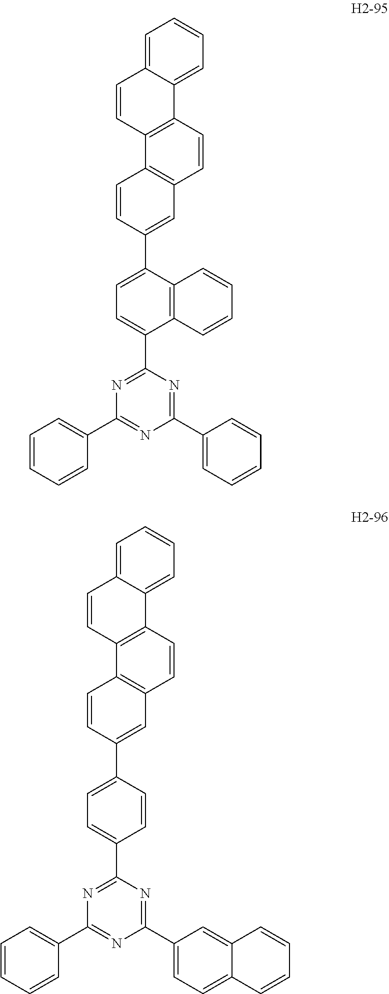 [SMILES: c1ccc(-c2nc(-c3ccccc3)nc(-c3ccc(-c4ccc5c(ccc6c7ccccc7ccc56)c4)c4ccccc34)n2)cc1 ; c1ccc(-c2nc(-c3ccc(-c4ccc5c(ccc6c7ccccc7ccc56)c4)cc3)nc(-c3ccc4ccccc4c3)n2)cc1]

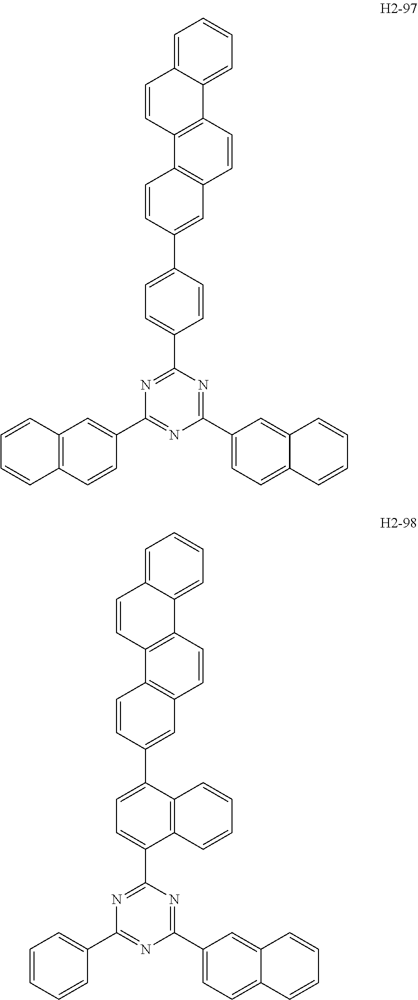 [SMILES: c1ccc2cc(-c3nc(-c4ccc(-c5ccc6c(ccc7c8ccccc8ccc67)c5)cc4)nc(-c4ccc5ccccc5c4)n3)ccc2c1 ; c1ccc(-c2nc(-c3ccc4ccccc4c3)nc(-c3ccc(-c4ccc5c(ccc6c7ccccc7ccc56)c4)c4ccccc34)n2)cc1]

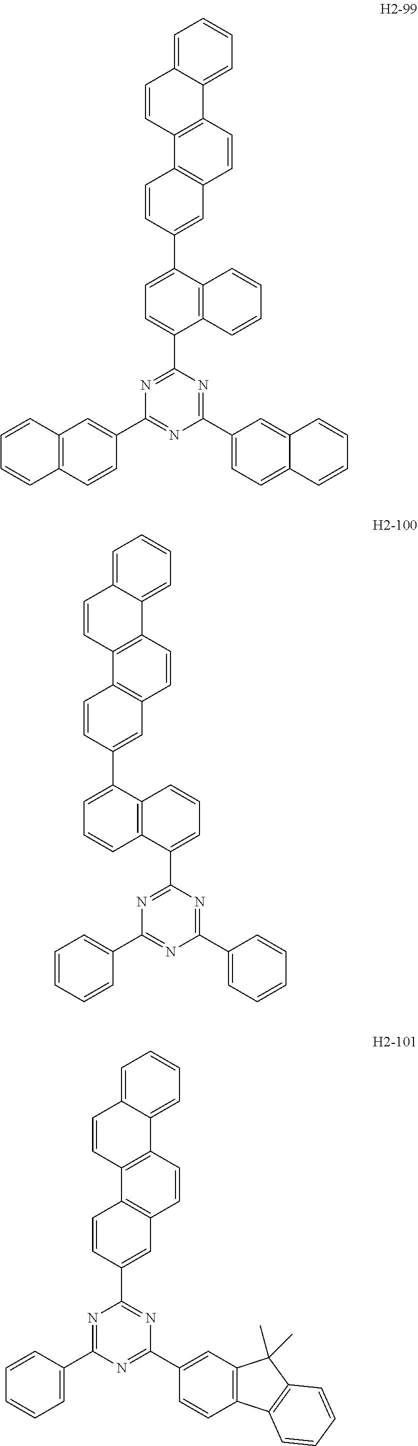 [SMILES: c1ccc2cc(-c3nc(-c4ccc5ccccc5c4)nc(-c4ccc(-c5ccc6c(ccc7c8ccccc8ccc67)c5)c5ccccc45)n3)ccc2c1 ; c1ccc(-c2nc(-c3ccccc3)nc(-c3cccc4c(-c5ccc6c(ccc7c8ccccc8ccc67)c5)cccc34)n2)cc1 ; CC1(C)c2ccccc2-c2ccc(-c3nc(-c4ccccc4)nc(-c4ccc5c(ccc6c7ccccc7ccc56)c4)n3)cc21]

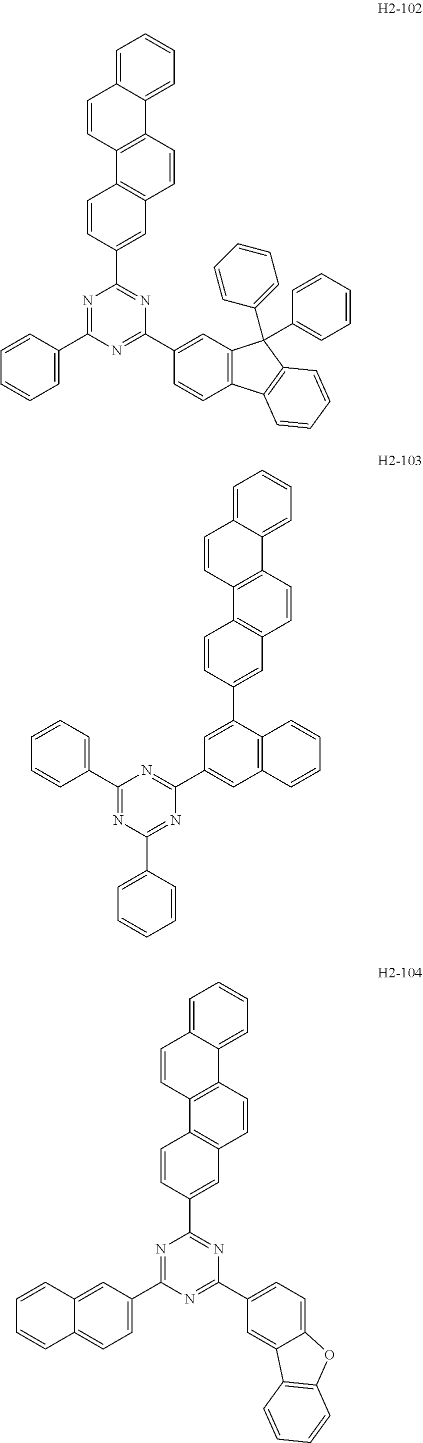 [SMILES: c1ccc(-c2nc(-c3ccc4c(c3)C(c3ccccc3)(c3ccccc3)c3ccccc3-4)nc(-c3ccc4c(ccc5c6ccccc6ccc45)c3)n2)cc1 ; c1ccc(-c2nc(-c3ccccc3)nc(-c3cc(-c4ccc5c(ccc6c7ccccc7ccc56)c4)c4ccccc4c3)n2)cc1 ; c1ccc2cc(-c3nc(-c4ccc5c(ccc6c7ccccc7ccc56)c4)nc(-c4ccc5oc6ccccc6c5c4)n3)ccc2c1]

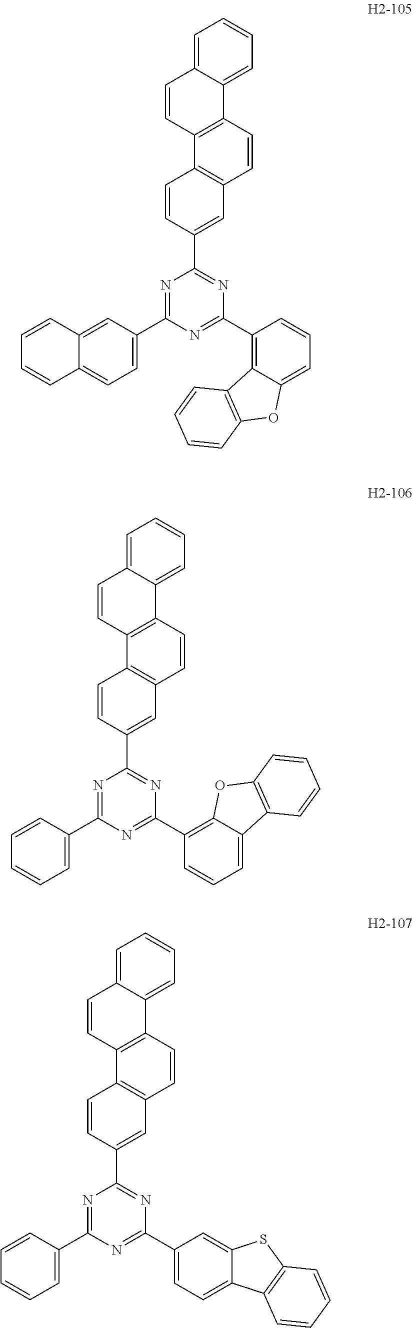 [SMILES: c1ccc2cc(-c3nc(-c4ccc5c(ccc6c7ccccc7ccc56)c4)nc(-c4cccc5oc6ccccc6c45)n3)ccc2c1 ; c1ccc(-c2nc(-c3ccc4c(ccc5c6ccccc6ccc45)c3)nc(-c3cccc4c3oc3ccccc34)n2)cc1 ; c1ccc(-c2nc(-c3ccc4c(ccc5c6ccccc6ccc45)c3)nc(-c3ccc4c(c3)sc3ccccc34)n2)cc1]

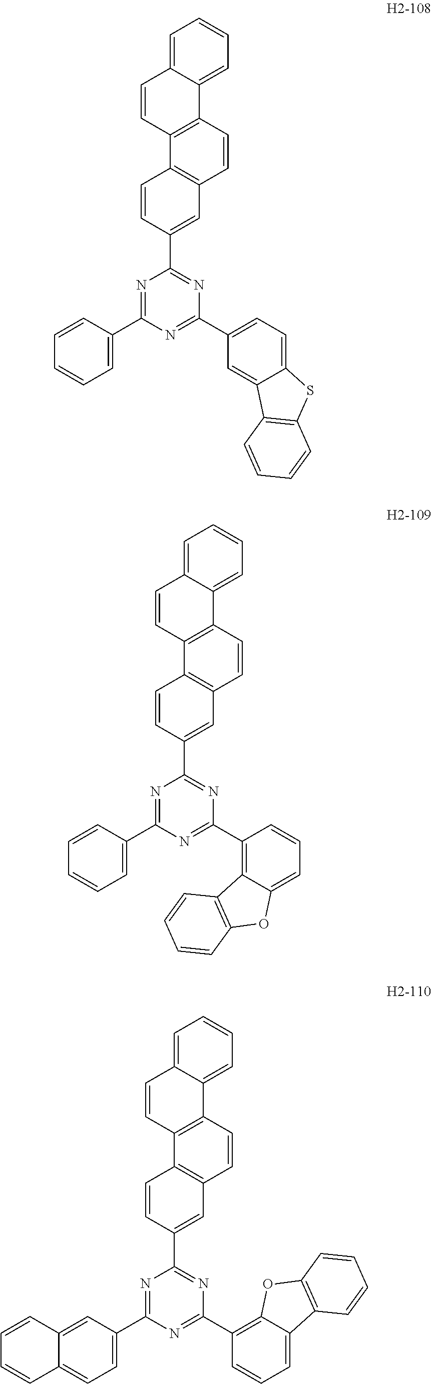 [SMILES: c1ccc(-c2nc(-c3ccc4c(ccc5c6ccccc6ccc45)c3)nc(-c3ccc4sc5ccccc5c4c3)n2)cc1 ; c1ccc(-c2nc(-c3ccc4c(ccc5c6ccccc6ccc45)c3)nc(-c3cccc4oc5ccccc5c34)n2)cc1 ; c1ccc2cc(-c3nc(-c4ccc5c(ccc6c7ccccc7ccc56)c4)nc(-c4cccc5c4oc4ccccc45)n3)ccc2c1]

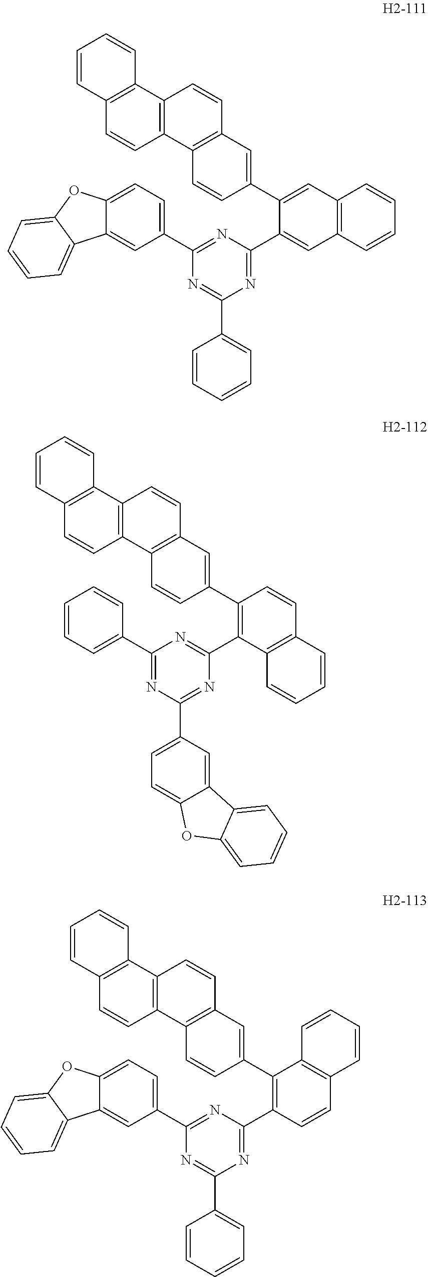 [SMILES: c1ccc(-c2nc(-c3ccc4oc5ccccc5c4c3)nc(-c3cc4ccccc4cc3-c3ccc4c(ccc5c6ccccc6ccc45)c3)n2)cc1 ; c1ccc(-c2nc(-c3ccc4oc5ccccc5c4c3)nc(-c3c(-c4ccc5c(ccc6c7ccccc7ccc56)c4)ccc4ccccc34)n2)cc1 ; c1ccc(-c2nc(-c3ccc4oc5ccccc5c4c3)nc(-c3ccc4ccccc4c3-c3ccc4c(ccc5c6ccccc6ccc45)c3)n2)cc1]

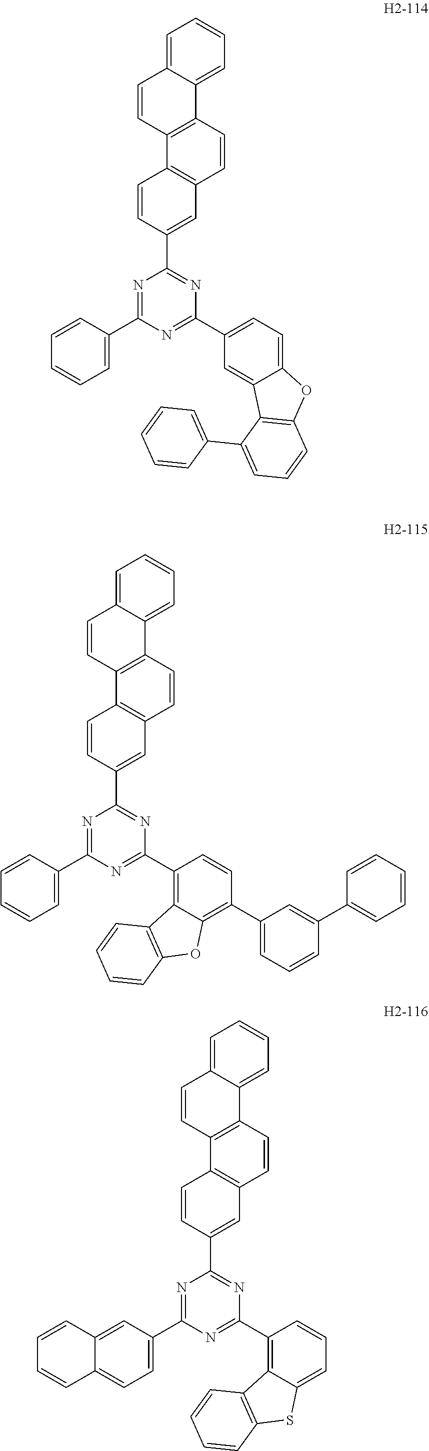 [SMILES: c1ccc(-c2nc(-c3ccc4c(ccc5c6ccccc6ccc45)c3)nc(-c3ccc4oc5cccc(-c6ccccc6)c5c4c3)n2)cc1 ; c1ccc(-c2cccc(-c3ccc(-c4nc(-c5ccccc5)nc(-c5ccc6c(ccc7c8ccccc8ccc67)c5)n4)c4c3oc3ccccc34)c2)cc1 ; c1ccc2cc(-c3nc(-c4ccc5c(ccc6c7ccccc7ccc56)c4)nc(-c4cccc5sc6ccccc6c45)n3)ccc2c1]

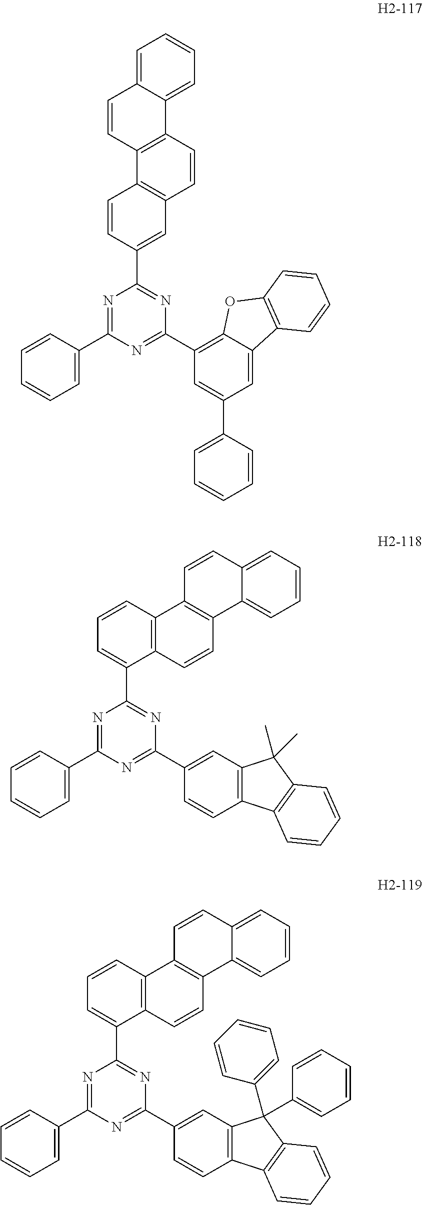 [SMILES: c1ccc(-c2cc(-c3nc(-c4ccccc4)nc(-c4ccc5c(ccc6c7ccccc7ccc56)c4)n3)c3oc4ccccc4c3c2)cc1 ; CC1(C)c2ccccc2-c2ccc(-c3nc(-c4ccccc4)nc(-c4cccc5c4ccc4c6ccccc6ccc54)n3)cc21 ; c1ccc(-c2nc(-c3ccc4c(c3)C(c3ccccc3)(c3ccccc3)c3ccccc3-4)nc(-c3cccc4c3ccc3c5ccccc5ccc43)n2)cc1]

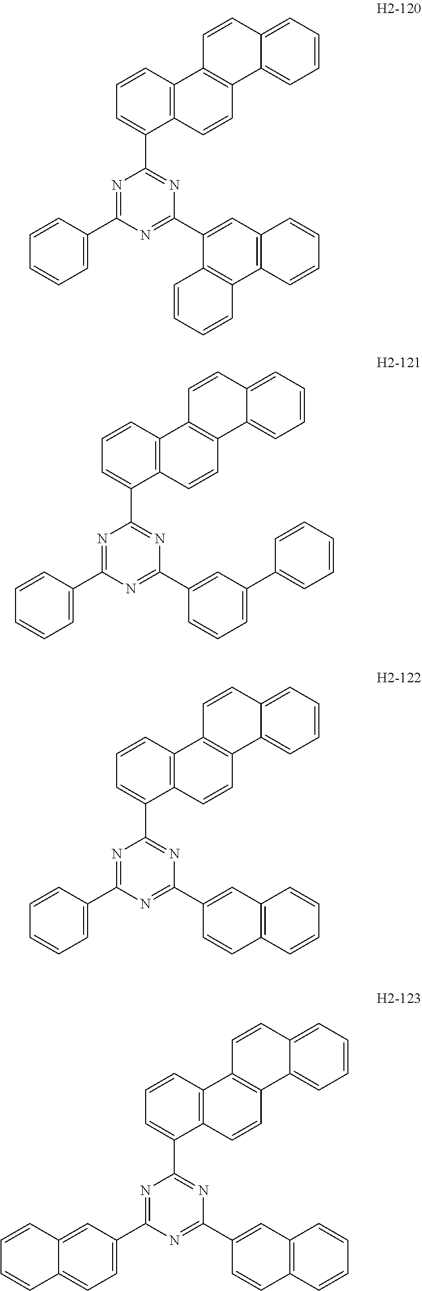

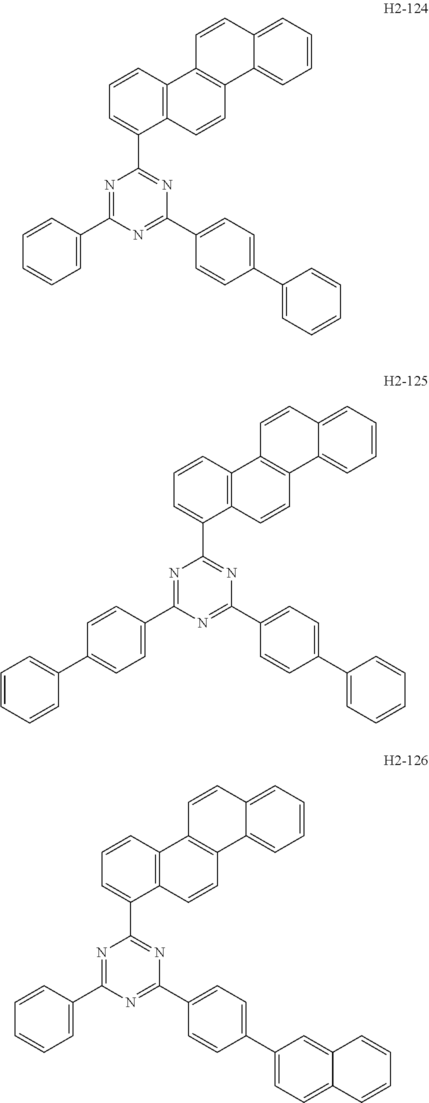 [SMILES: c1ccc(-c2ccc(-c3nc(-c4ccccc4)nc(-c4cccc5c4ccc4c6ccccc6ccc54)n3)cc2)cc1 ; c1ccc(-c2ccc(-c3nc(-c4ccc(-c5ccccc5)cc4)nc(-c4cccc5c4ccc4c6ccccc6ccc54)n3)cc2)cc1 ; c1ccc(-c2nc(-c3ccc(-c4ccc5ccccc5c4)cc3)nc(-c3cccc4c3ccc3c5ccccc5ccc43)n2)cc1]

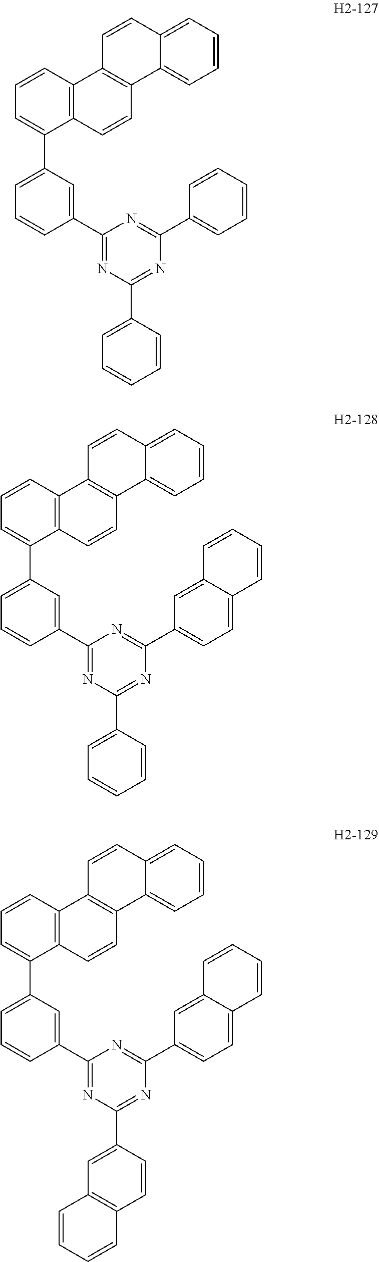

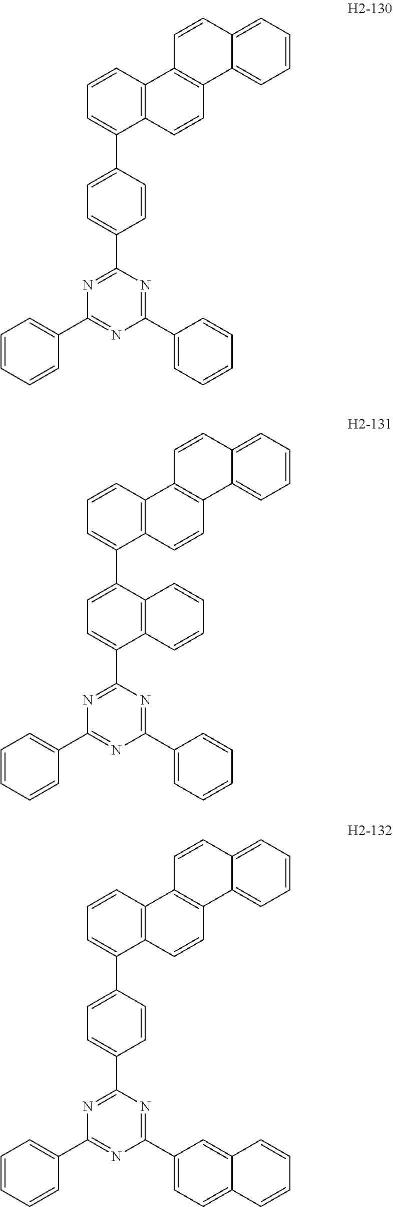

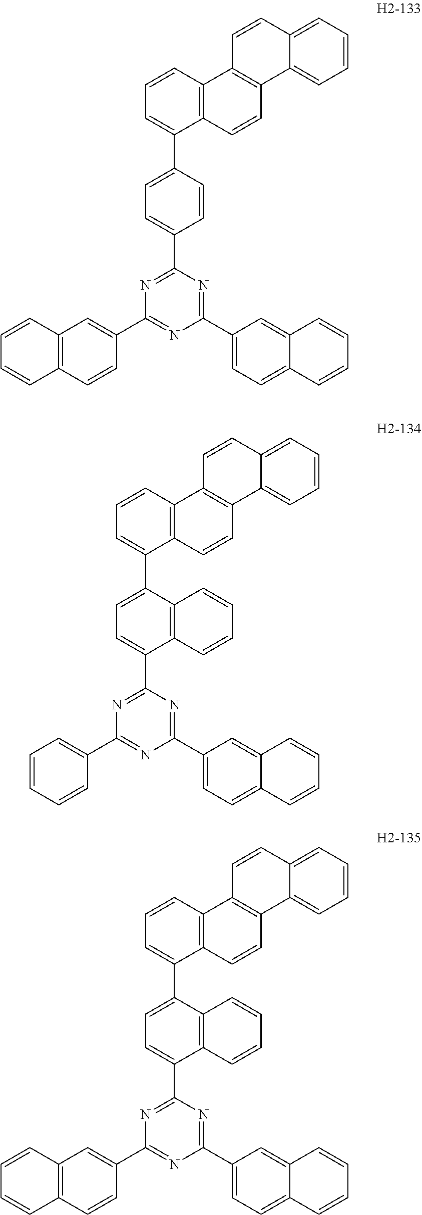 [SMILES: c1ccc2cc(-c3nc(-c4ccc(-c5cccc6c5ccc5c7ccccc7ccc65)cc4)nc(-c4ccc5ccccc5c4)n3)ccc2c1 ; c1ccc(-c2nc(-c3ccc4ccccc4c3)nc(-c3ccc(-c4cccc5c4ccc4c6ccccc6ccc54)c4ccccc34)n2)cc1 ; c1ccc2cc(-c3nc(-c4ccc5ccccc5c4)nc(-c4ccc(-c5cccc6c5ccc5c7ccccc7ccc65)c5ccccc45)n3)ccc2c1]

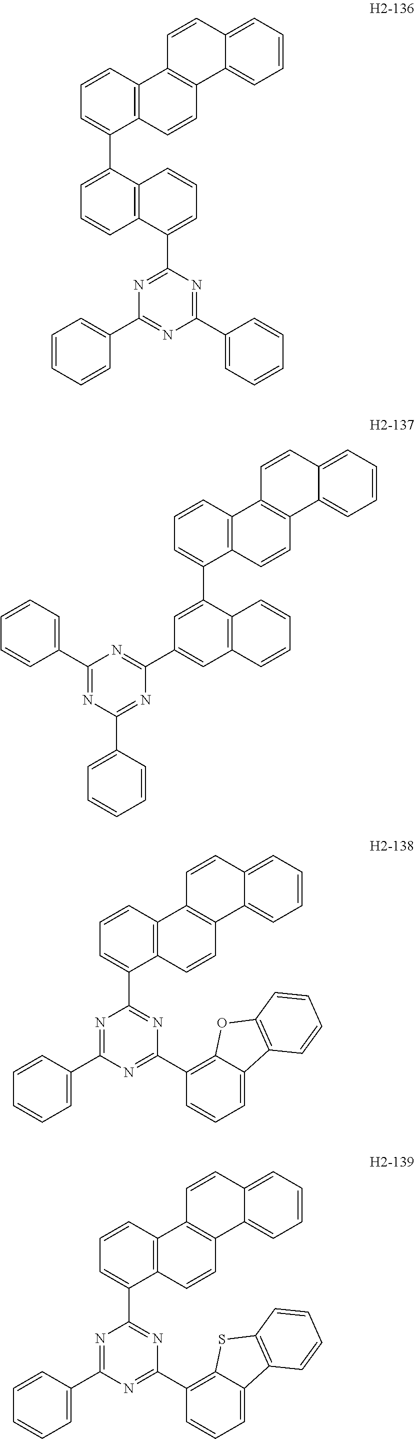 [SMILES: c1ccc(-c2nc(-c3ccccc3)nc(-c3cccc4c(-c5cccc6c5ccc5c7ccccc7ccc65)cccc34)n2)cc1 ; c1ccc(-c2nc(-c3ccccc3)nc(-c3cc(-c4cccc5c4ccc4c6ccccc6ccc54)c4ccccc4c3)n2)cc1 ; c1ccc(-c2nc(-c3cccc4c3ccc3c5ccccc5ccc43)nc(-c3cccc4c3oc3ccccc34)n2)cc1 ; c1ccc(-c2nc(-c3cccc4c3ccc3c5ccccc5ccc43)nc(-c3cccc4c3sc3ccccc34)n2)cc1]

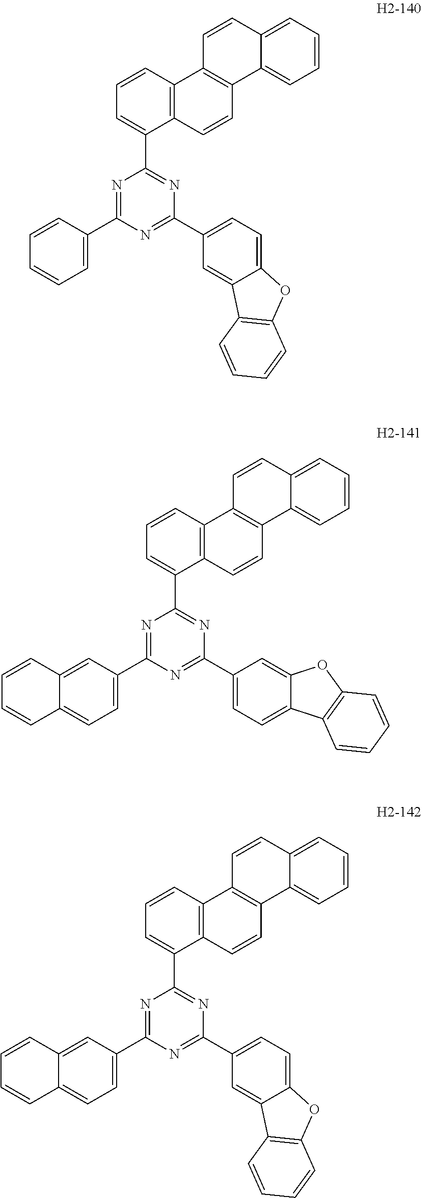 [SMILES: c1ccc(-c2nc(-c3ccc4oc5ccccc5c4c3)nc(-c3cccc4c3ccc3c5ccccc5ccc43)n2)cc1 ; c1ccc2cc(-c3nc(-c4ccc5c(c4)oc4ccccc45)nc(-c4cccc5c4ccc4c6ccccc6ccc54)n3)ccc2c1 ; c1ccc2cc(-c3nc(-c4ccc5oc6ccccc6c5c4)nc(-c4cccc5c4ccc4c6ccccc6ccc54)n3)ccc2c1]

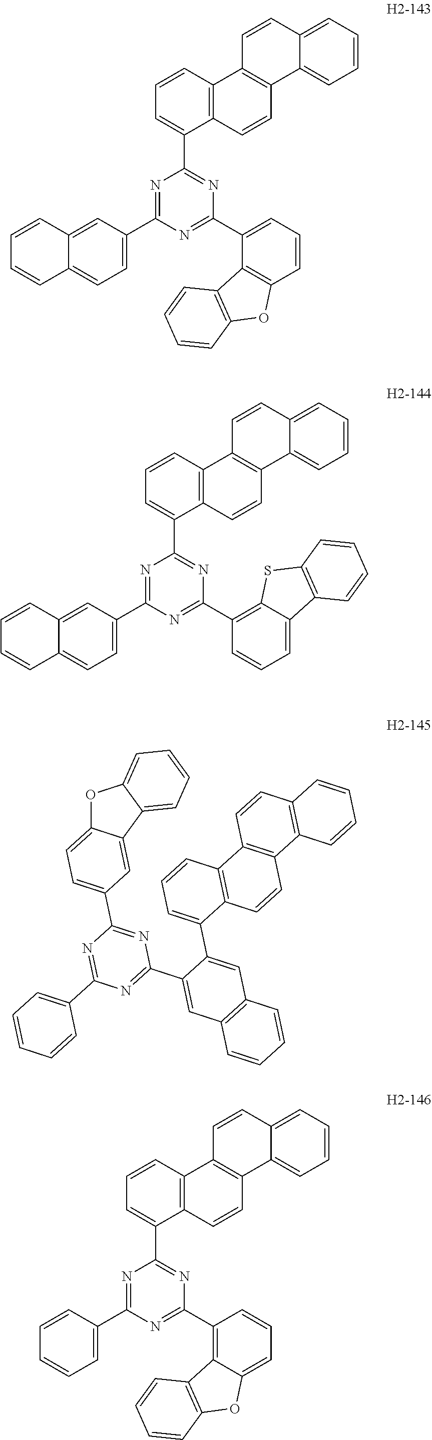 [SMILES: c1ccc2cc(-c3nc(-c4cccc5c4ccc4c6ccccc6ccc54)nc(-c4cccc5oc6ccccc6c45)n3)ccc2c1 ; c1ccc2cc(-c3nc(-c4cccc5c4ccc4c6ccccc6ccc54)nc(-c4cccc5c4sc4ccccc45)n3)ccc2c1 ; c1ccc(-c2nc(-c3ccc4oc5ccccc5c4c3)nc(-c3cc4ccccc4cc3-c3cccc4c3ccc3c5ccccc5ccc43)n2)cc1 ; c1ccc(-c2nc(-c3cccc4c3ccc3c5ccccc5ccc43)nc(-c3cccc4oc5ccccc5c34)n2)cc1]

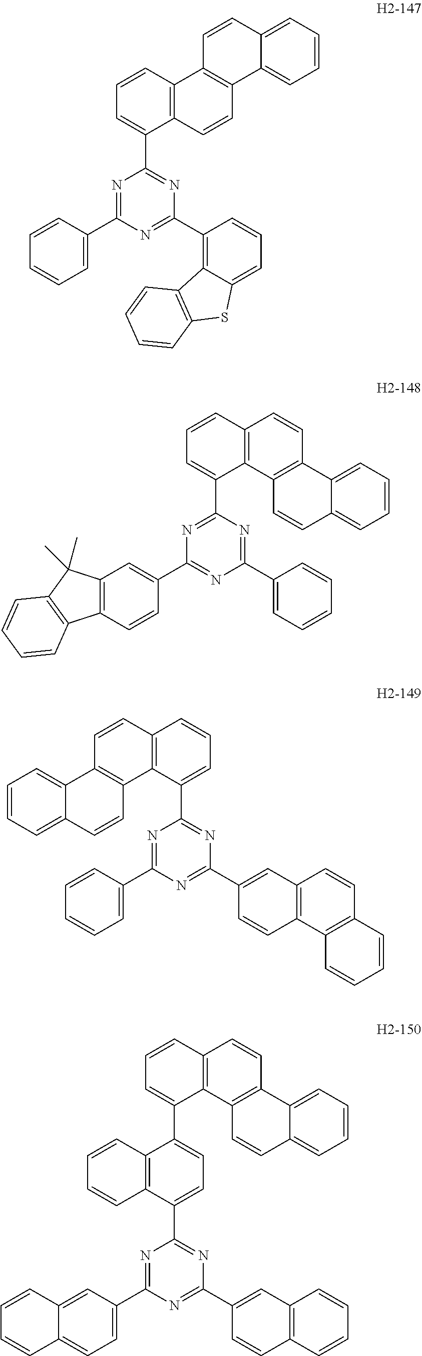 [SMILES: c1ccc(-c2nc(-c3cccc4c3ccc3c5ccccc5ccc43)nc(-c3cccc4sc5ccccc5c34)n2)cc1 ; CC1(C)c2ccccc2-c2ccc(-c3nc(-c4ccccc4)nc(-c4cccc5ccc6c7ccccc7ccc6c45)n3)cc21 ; c1ccc(-c2nc(-c3ccc4c(ccc5ccccc54)c3)nc(-c3cccc4ccc5c6ccccc6ccc5c34)n2)cc1 ; c1ccc2cc(-c3nc(-c4ccc5ccccc5c4)nc(-c4ccc(-c5cccc6ccc7c8ccccc8ccc7c56)c5ccccc45)n3)ccc2c1]

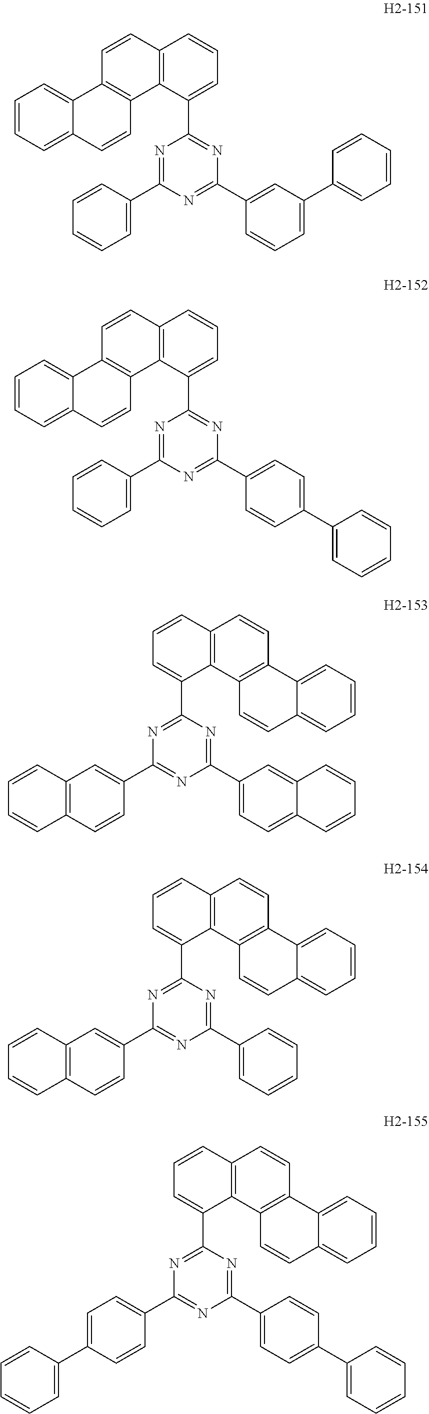 [SMILES: c1ccc(-c2cccc(-c3nc(-c4ccccc4)nc(-c4cccc5ccc6c7ccccc7ccc6c45)n3)c2)cc1 ; c1ccc(-c2ccc(-c3nc(-c4ccccc4)nc(-c4cccc5ccc6c7ccccc7ccc6c45)n3)cc2)cc1 ; c1ccc2cc(-c3nc(-c4ccc5ccccc5c4)nc(-c4cccc5ccc6c7ccccc7ccc6c45)n3)ccc2c1 ; c1ccc(-c2nc(-c3ccc4ccccc4c3)nc(-c3cccc4ccc5c6ccccc6ccc5c34)n2)cc1 ; c1ccc(-c2ccc(-c3nc(-c4ccc(-c5ccccc5)cc4)nc(-c4cccc5ccc6c7ccccc7ccc6c45)n3)cc2)cc1]

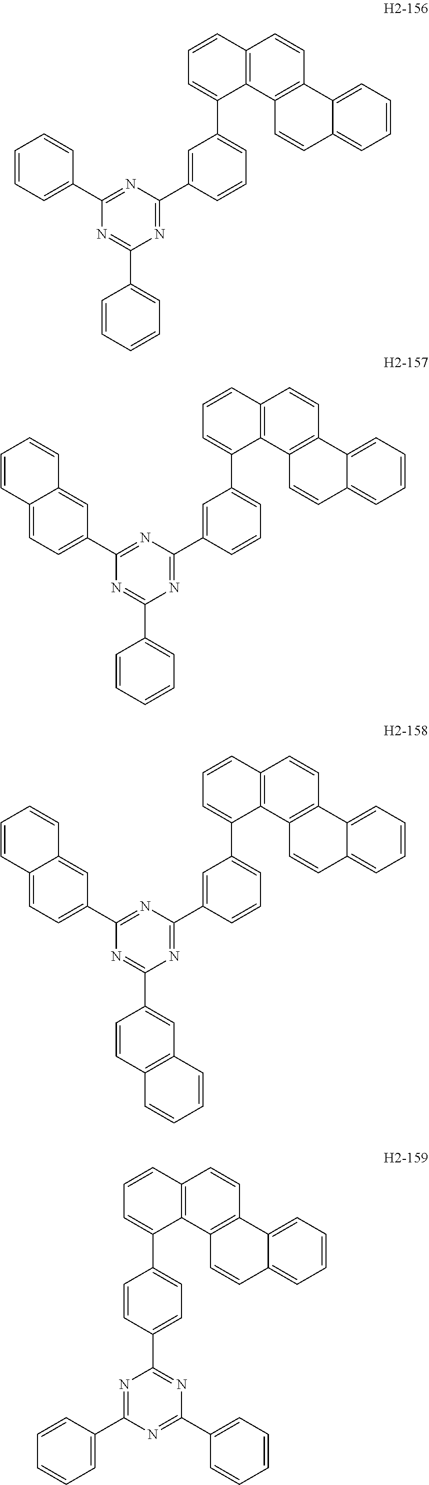 [SMILES: c1ccc(-c2nc(-c3ccccc3)nc(-c3cccc(-c4cccc5ccc6c7ccccc7ccc6c45)c3)n2)cc1 ; c1ccc(-c2nc(-c3cccc(-c4cccc5ccc6c7ccccc7ccc6c45)c3)nc(-c3ccc4ccccc4c3)n2)cc1 ; c1cc(-c2nc(-c3ccc4ccccc4c3)nc(-c3ccc4ccccc4c3)n2)cc(-c2cccc3ccc4c5ccccc5ccc4c23)c1 ; c1ccc(-c2nc(-c3ccccc3)nc(-c3ccc(-c4cccc5ccc6c7ccccc7ccc6c45)cc3)n2)cc1]

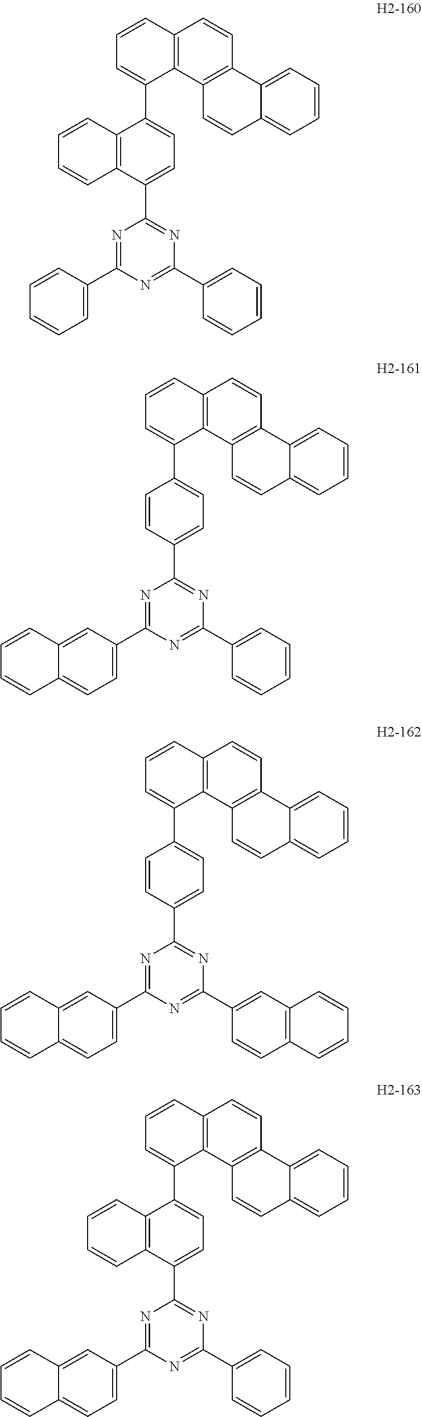

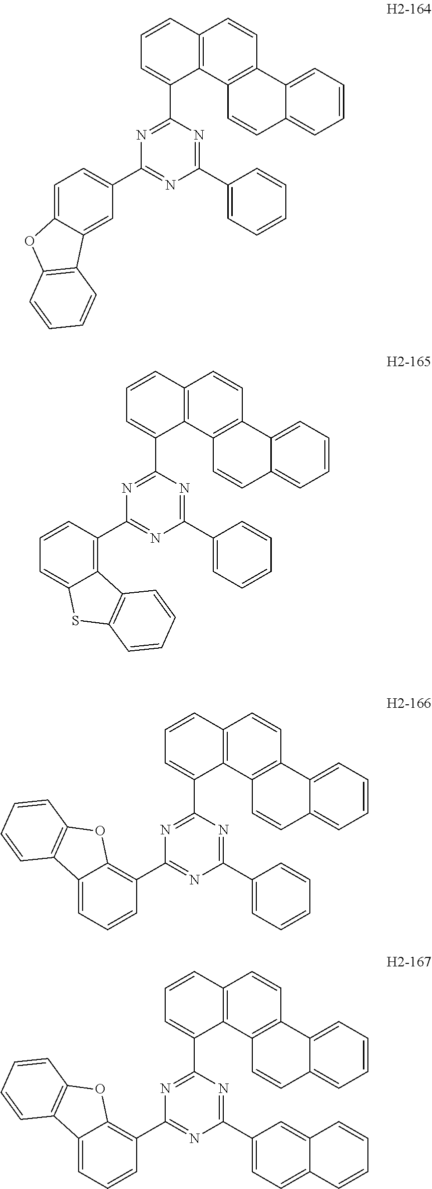 [SMILES: c1ccc(-c2nc(-c3ccc4oc5ccccc5c4c3)nc(-c3cccc4ccc5c6ccccc6ccc5c34)n2)cc1 ; c1ccc(-c2nc(-c3cccc4ccc5c6ccccc6ccc5c34)nc(-c3cccc4sc5ccccc5c34)n2)cc1 ; c1ccc(-c2nc(-c3cccc4c3oc3ccccc34)nc(-c3cccc4ccc5c6ccccc6ccc5c34)n2)cc1 ; c1ccc2cc(-c3nc(-c4cccc5c4oc4ccccc45)nc(-c4cccc5ccc6c7ccccc7ccc6c45)n3)ccc2c1]

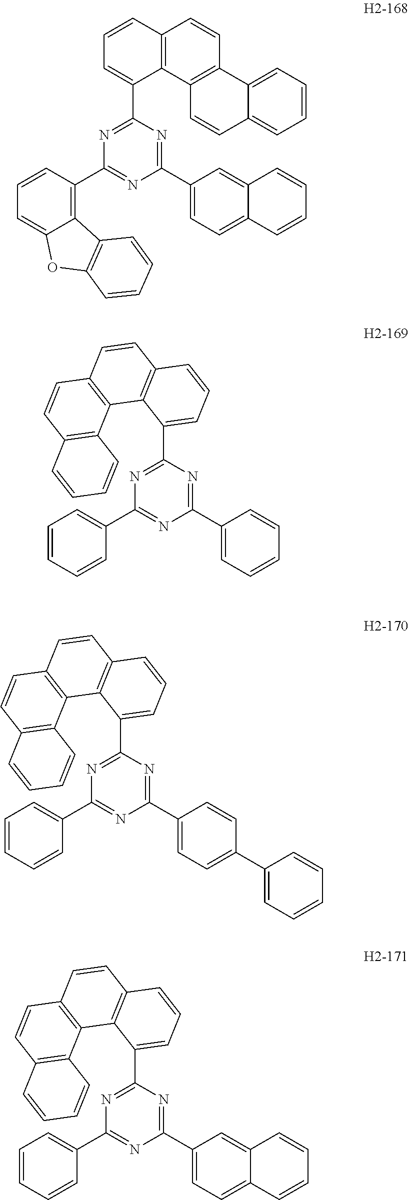 [SMILES: c1ccc2cc(-c3nc(-c4cccc5ccc6c7ccccc7ccc6c45)nc(-c4cccc5oc6ccccc6c45)n3)ccc2c1 ; c1ccc(-c2nc(-c3ccccc3)nc(-c3cccc4ccc5ccc6ccccc6c5c34)n2)cc1 ; c1ccc(-c2ccc(-c3nc(-c4ccccc4)nc(-c4cccc5ccc6ccc7ccccc7c6c45)n3)cc2)cc1 ; c1ccc(-c2nc(-c3ccc4ccccc4c3)nc(-c3cccc4ccc5ccc6ccccc6c5c34)n2)cc1]

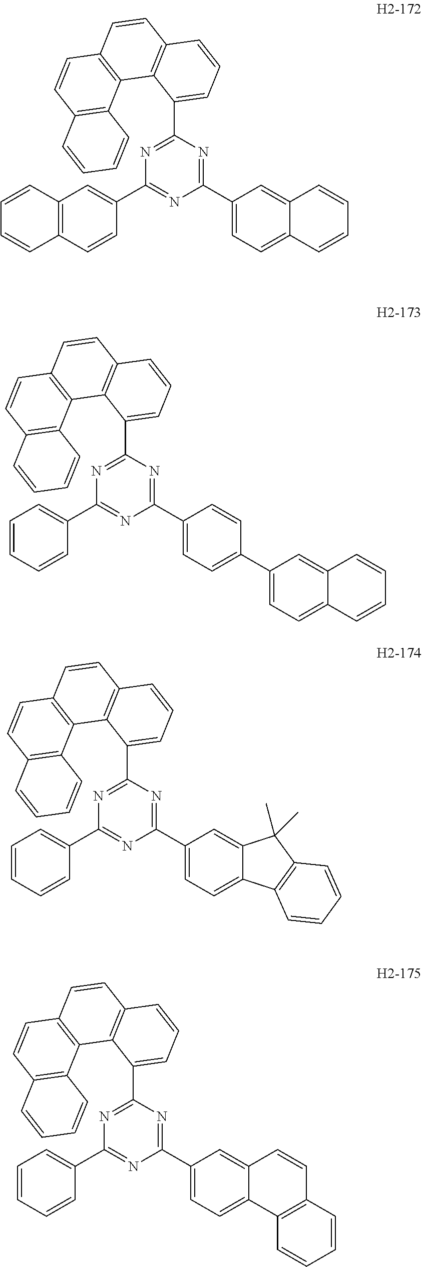 [SMILES: c1ccc2cc(-c3nc(-c4ccc5ccccc5c4)nc(-c4cccc5ccc6ccc7ccccc7c6c45)n3)ccc2c1 ; c1ccc(-c2nc(-c3ccc(-c4ccc5ccccc5c4)cc3)nc(-c3cccc4ccc5ccc6ccccc6c5c34)n2)cc1 ; CC1(C)c2ccccc2-c2ccc(-c3nc(-c4ccccc4)nc(-c4cccc5ccc6ccc7ccccc7c6c45)n3)cc21 ; c1ccc(-c2nc(-c3ccc4c(ccc5ccccc54)c3)nc(-c3cccc4ccc5ccc6ccccc6c5c34)n2)cc1]

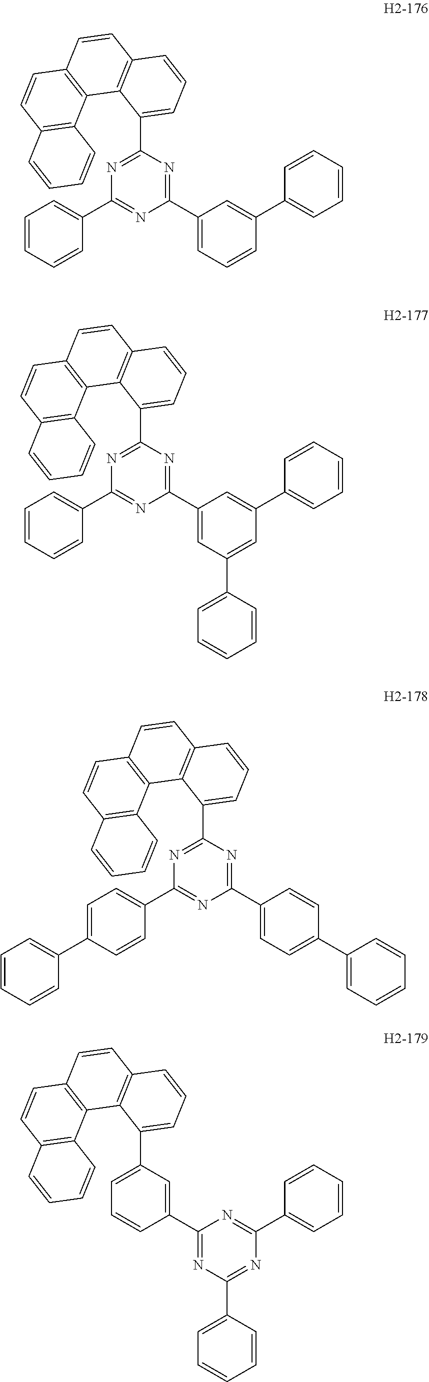 [SMILES: c1ccc(-c2cccc(-c3nc(-c4ccccc4)nc(-c4cccc5ccc6ccc7ccccc7c6c45)n3)c2)cc1 ; c1ccc(-c2cc(-c3ccccc3)cc(-c3nc(-c4ccccc4)nc(-c4cccc5ccc6ccc7ccccc7c6c45)n3)c2)cc1 ; c1ccc(-c2ccc(-c3nc(-c4ccc(-c5ccccc5)cc4)nc(-c4cccc5ccc6ccc7ccccc7c6c45)n3)cc2)cc1 ; c1ccc(-c2nc(-c3ccccc3)nc(-c3cccc(-c4cccc5ccc6ccc7ccccc7c6c45)c3)n2)cc1]

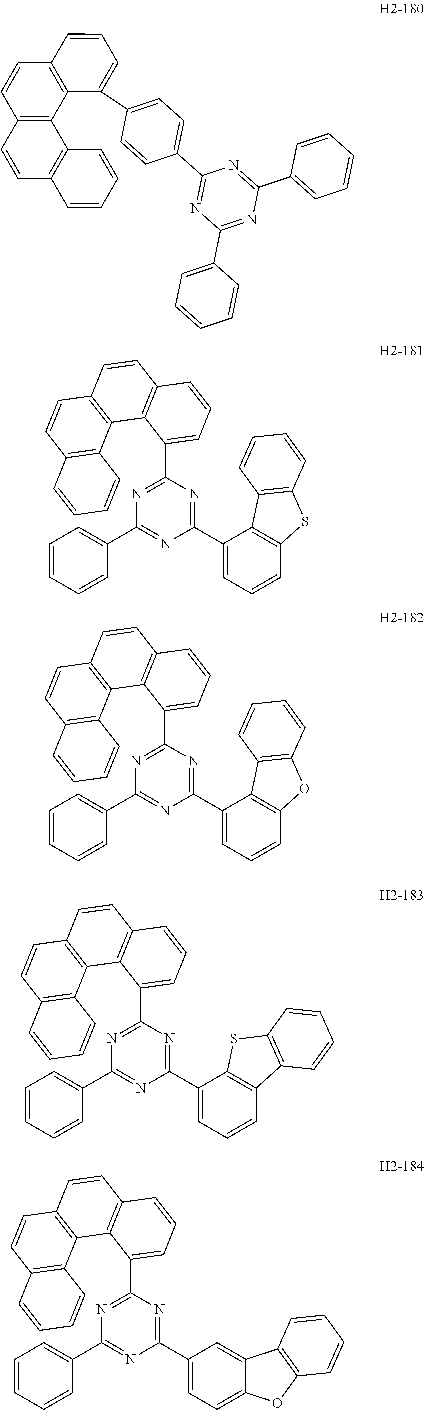

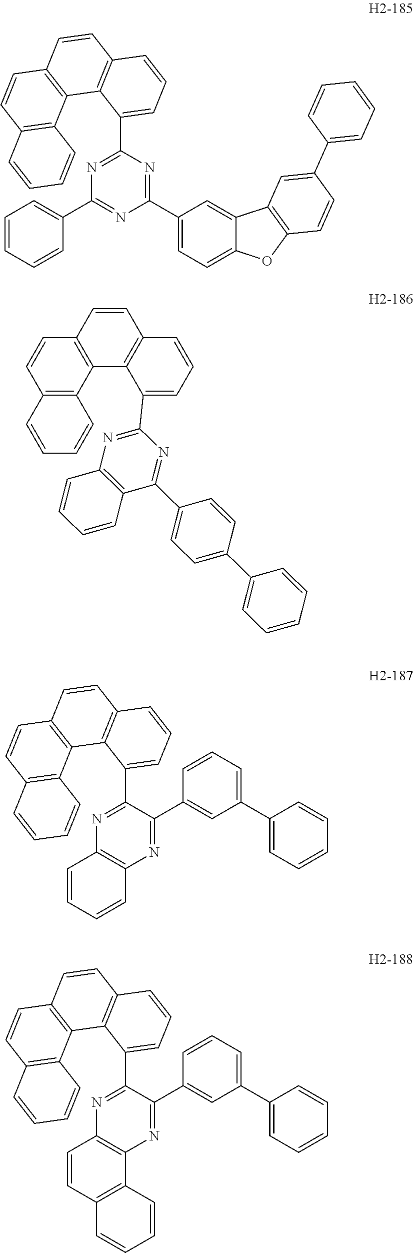 [SMILES: c1ccc(-c2ccc3oc4ccc(-c5nc(-c6ccccc6)nc(-c6cccc7ccc8ccc9ccccc9c8c67)n5)cc4c3c2)cc1 ; c1ccc(-c2ccc(-c3nc(-c4cccc5ccc6ccc7ccccc7c6c45)nc4ccccc34)cc2)cc1 ; c1ccc(-c2cccc(-c3nc4ccccc4nc3-c3cccc4ccc5ccc6ccccc6c5c34)c2)cc1 ; c1ccc(-c2cccc(-c3nc4c(ccc5ccccc54)nc3-c3cccc4ccc5ccc6ccccc6c5c34)c2)cc1]

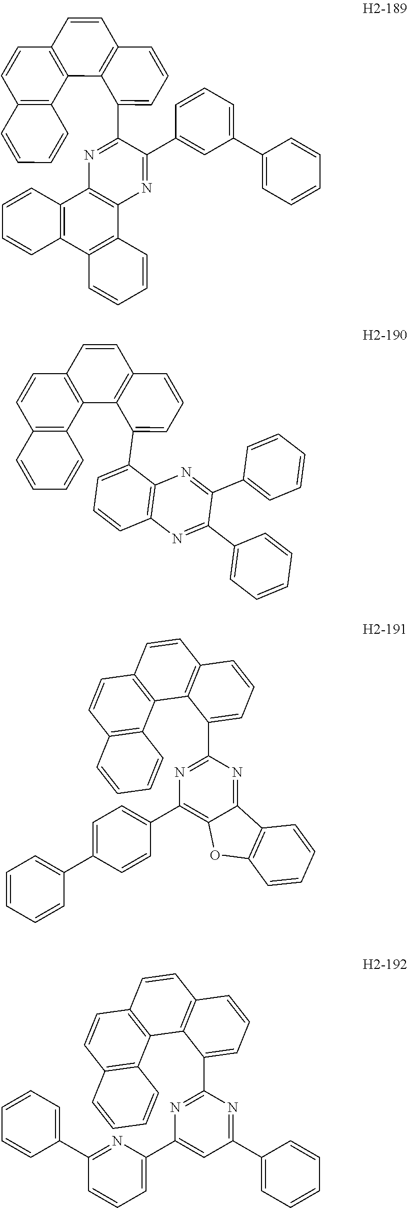

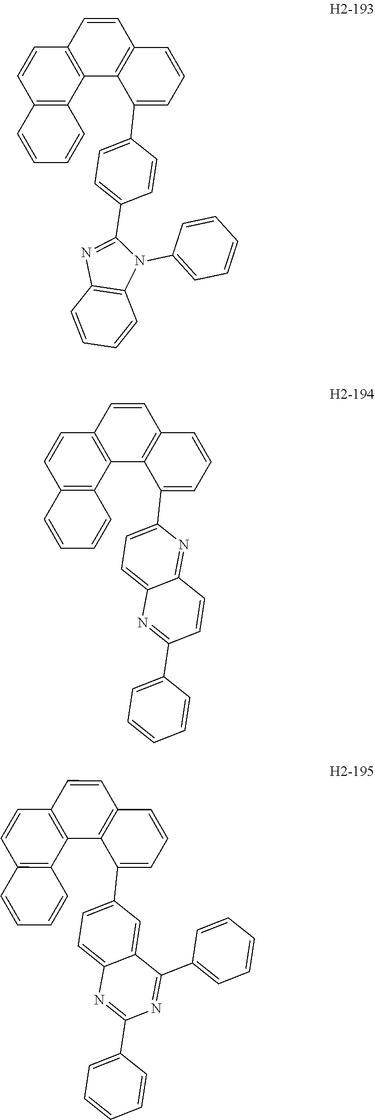 [SMILES: c1ccc(-n2c(-c3ccc(-c4cccc5ccc6ccc7ccccc7c6c45)cc3)nc3ccccc32)cc1 ; c1ccc(-c2ccc3nc(-c4cccc5ccc6ccc7ccccc7c6c45)ccc3n2)cc1 ; c1ccc(-c2nc(-c3ccccc3)c3cc(-c4cccc5ccc6ccc7ccccc7c6c45)ccc3n2)cc1]

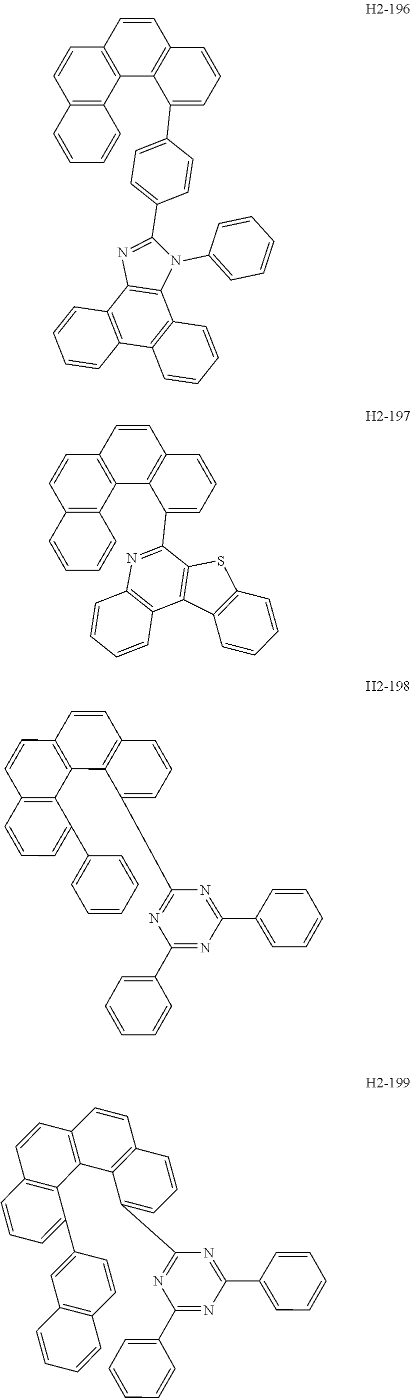 [SMILES: c1ccc(-n2c(-c3ccc(-c4cccc5ccc6ccc7ccccc7c6c45)cc3)nc3c4ccccc4c4ccccc4c32)cc1 ; c1ccc2c(c1)ccc1ccc3cccc(-c4nc5ccccc5c5c4sc4ccccc45)c3c12 ; c1ccc(-c2nc(-c3ccccc3)nc(-c3cccc4ccc5ccc6cccc(-c7ccccc7)c6c5c34)n2)cc1 ; c1ccc(-c2nc(-c3ccccc3)nc(-c3cccc4ccc5ccc6cccc(-c7ccc8ccccc8c7)c6c5c34)n2)cc1]

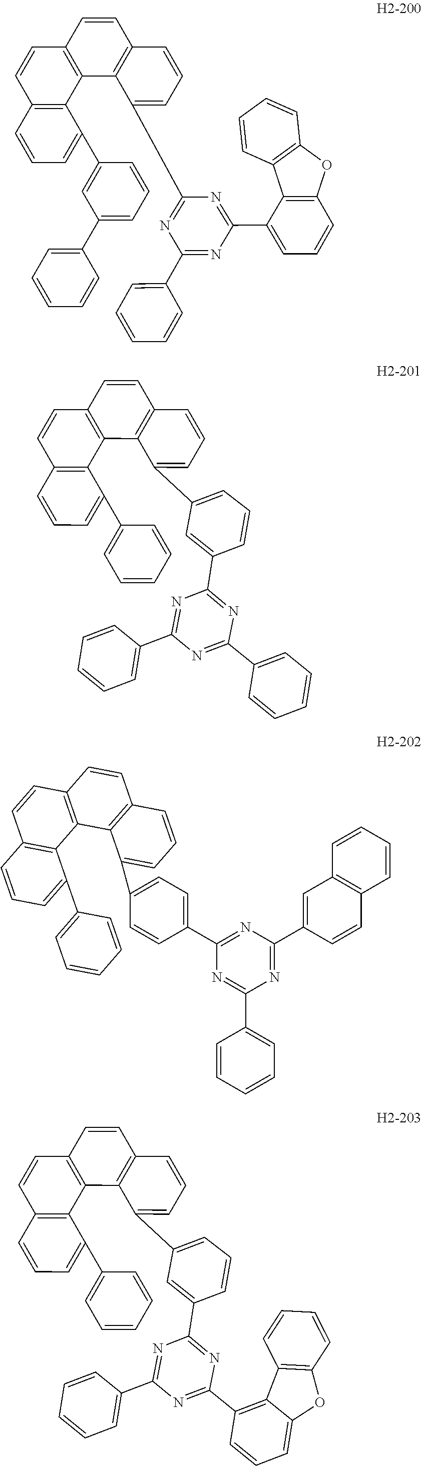

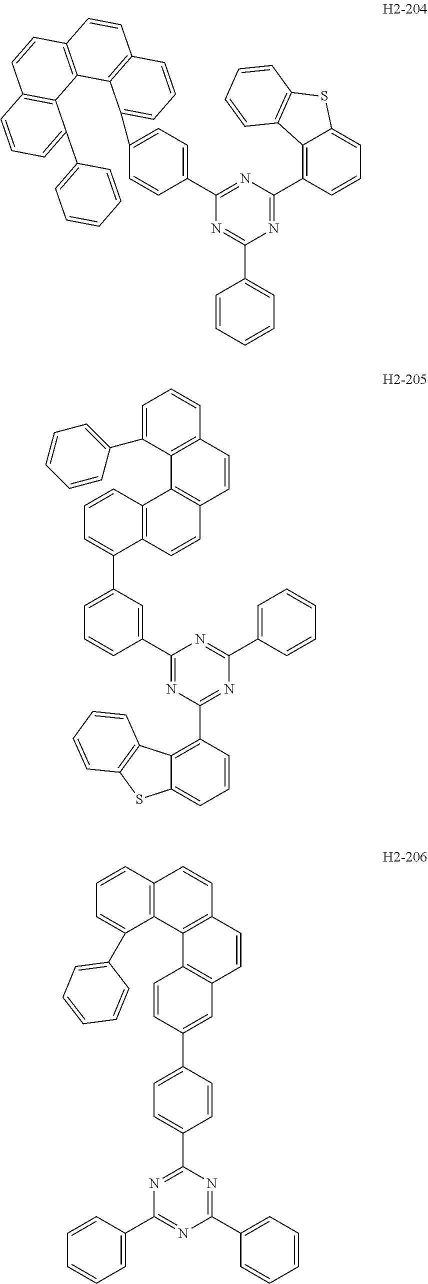 [SMILES: c1ccc(-c2nc(-c3ccc(-c4cccc5ccc6ccc7cccc(-c8ccccc8)c7c6c45)cc3)nc(-c3cccc4sc5ccccc5c34)n2)cc1 ; c1ccc(-c2nc(-c3cccc(-c4cccc5c4ccc4ccc6cccc(-c7ccccc7)c6c45)c3)nc(-c3cccc4sc5ccccc5c34)n2)cc1 ; c1ccc(-c2nc(-c3ccccc3)nc(-c3ccc(-c4ccc5c(ccc6ccc7cccc(-c8ccccc8)c7c65)c4)cc3)n2)cc1]

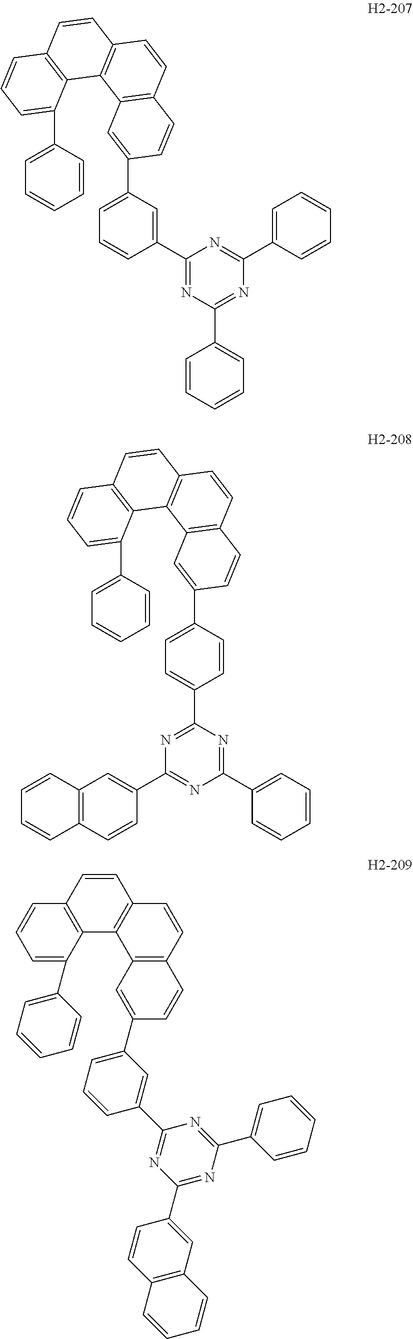

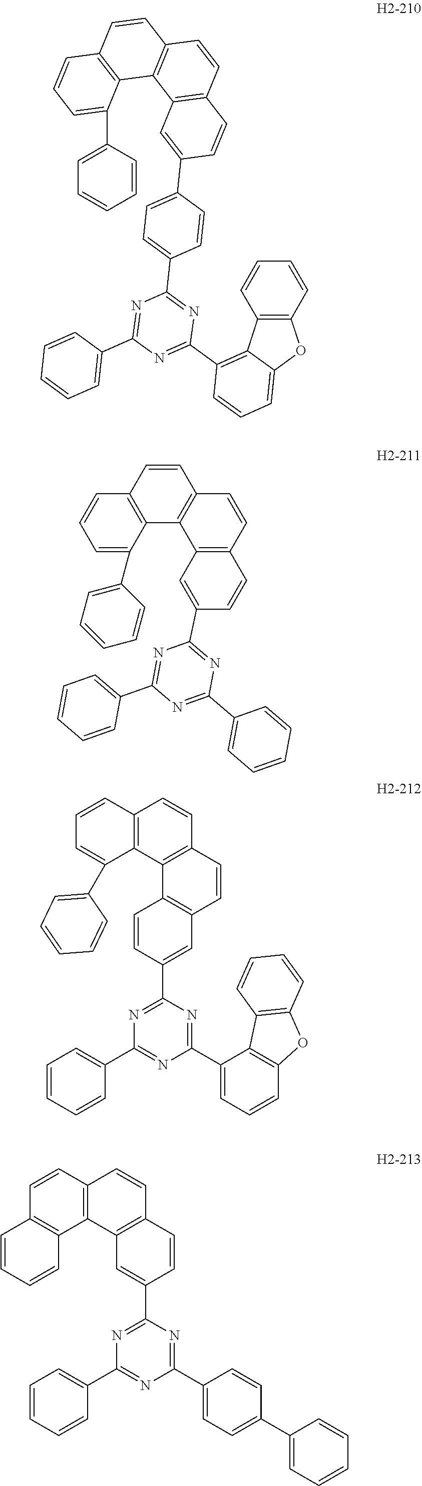 [SMILES: c1ccc(-c2nc(-c3ccc(-c4ccc5ccc6ccc7cccc(-c8ccccc8)c7c6c5c4)cc3)nc(-c3cccc4oc5ccccc5c34)n2)cc1 ; c1ccc(-c2nc(-c3ccccc3)nc(-c3ccc4ccc5ccc6cccc(-c7ccccc7)c6c5c4c3)n2)cc1 ; c1ccc(-c2nc(-c3ccc4c(ccc5ccc6cccc(-c7ccccc7)c6c54)c3)nc(-c3cccc4oc5ccccc5c34)n2)cc1 ; c1ccc(-c2ccc(-c3nc(-c4ccccc4)nc(-c4ccc5ccc6ccc7ccccc7c6c5c4)n3)cc2)cc1]

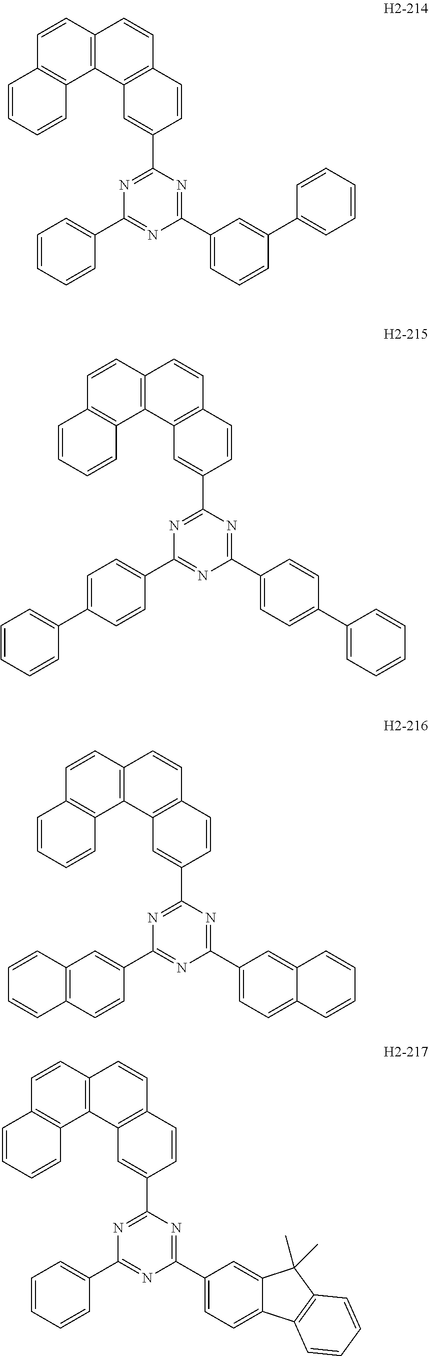 [SMILES: c1ccc(-c2cccc(-c3nc(-c4ccccc4)nc(-c4ccc5ccc6ccc7ccccc7c6c5c4)n3)c2)cc1 ; c1ccc(-c2ccc(-c3nc(-c4ccc(-c5ccccc5)cc4)nc(-c4ccc5ccc6ccc7ccccc7c6c5c4)n3)cc2)cc1 ; c1ccc2cc(-c3nc(-c4ccc5ccccc5c4)nc(-c4ccc5ccc6ccc7ccccc7c6c5c4)n3)ccc2c1 ; CC1(C)c2ccccc2-c2ccc(-c3nc(-c4ccccc4)nc(-c4ccc5ccc6ccc7ccccc7c6c5c4)n3)cc21]

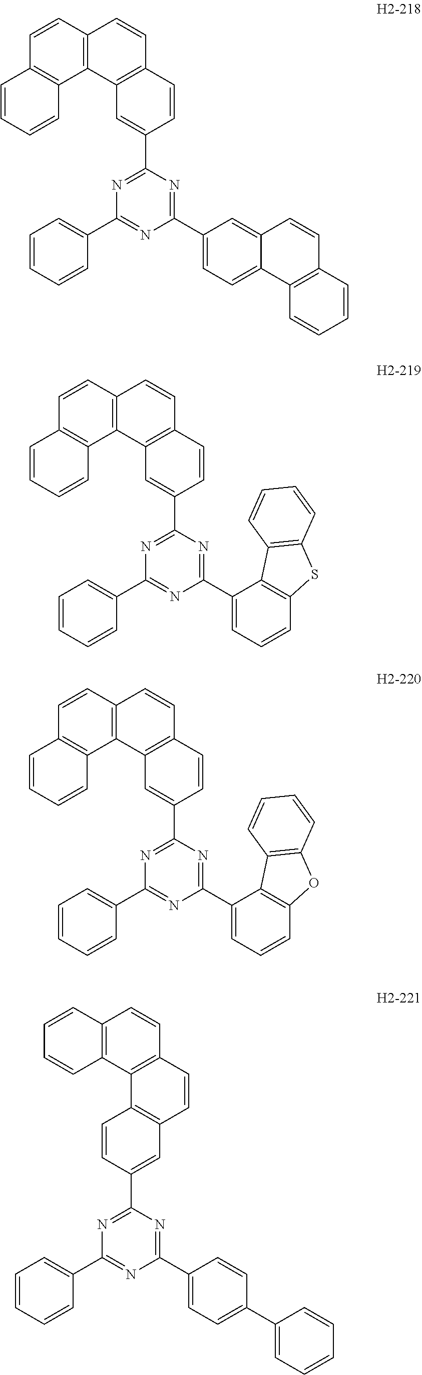

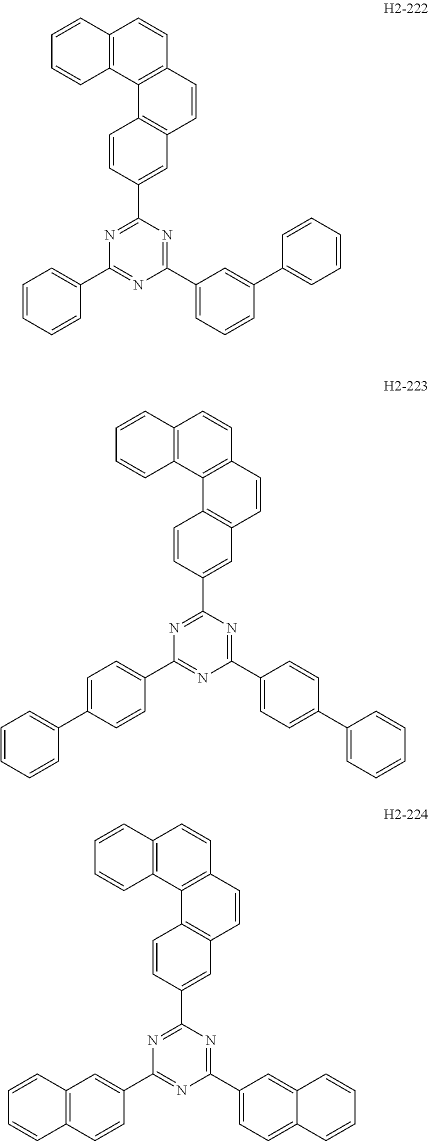 [SMILES: c1ccc(-c2cccc(-c3nc(-c4ccccc4)nc(-c4ccc5c(ccc6ccc7ccccc7c65)c4)n3)c2)cc1 ; c1ccc(-c2ccc(-c3nc(-c4ccc(-c5ccccc5)cc4)nc(-c4ccc5c(ccc6ccc7ccccc7c65)c4)n3)cc2)cc1 ; c1ccc2cc(-c3nc(-c4ccc5ccccc5c4)nc(-c4ccc5c(ccc6ccc7ccccc7c65)c4)n3)ccc2c1]

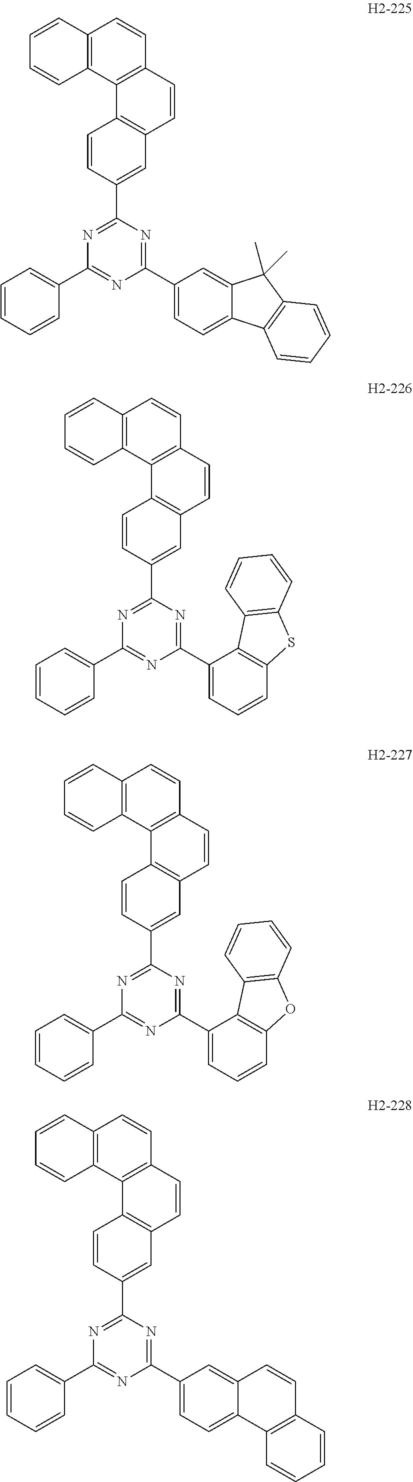

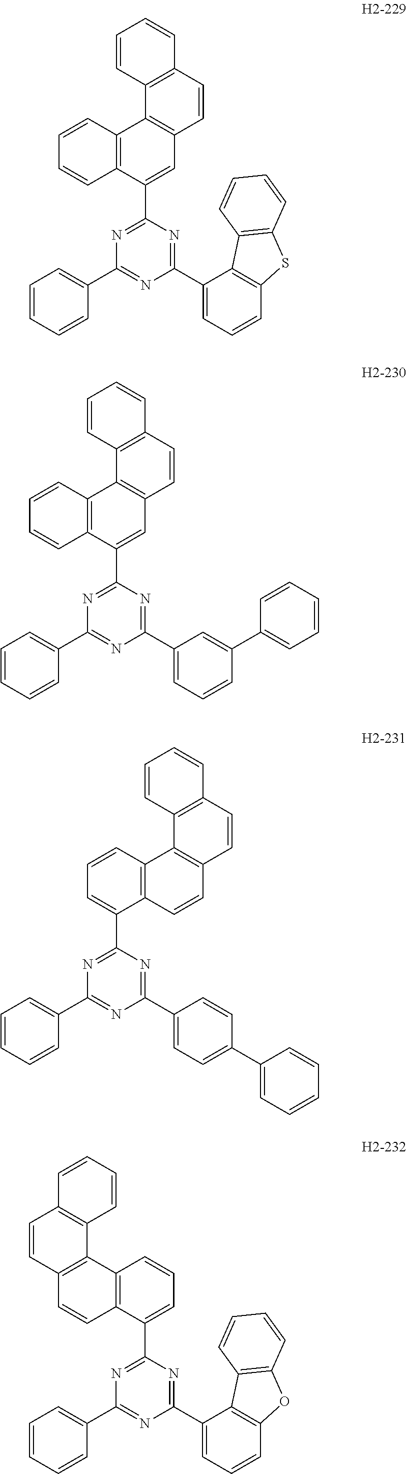 [SMILES: c1ccc(-c2nc(-c3cc4ccc5ccccc5c4c4ccccc34)nc(-c3cccc4sc5ccccc5c34)n2)cc1 ; c1ccc(-c2cccc(-c3nc(-c4ccccc4)nc(-c4cc5ccc6ccccc6c5c5ccccc45)n3)c2)cc1 ; c1ccc(-c2ccc(-c3nc(-c4ccccc4)nc(-c4cccc5c4ccc4ccc6ccccc6c45)n3)cc2)cc1 ; c1ccc(-c2nc(-c3cccc4c3ccc3ccc5ccccc5c34)nc(-c3cccc4oc5ccccc5c34)n2)cc1]

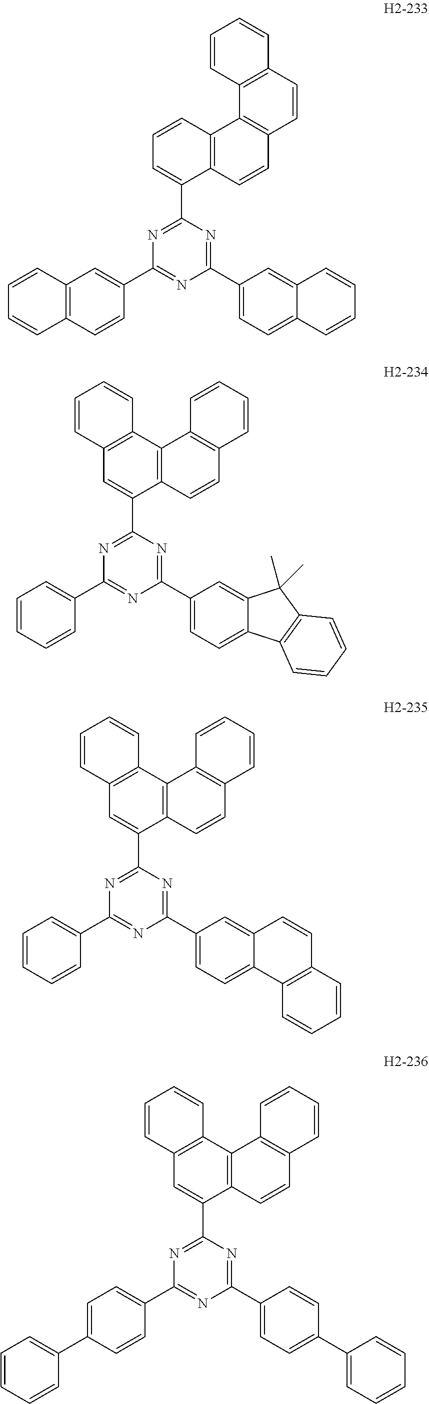 [SMILES: c1ccc2cc(-c3nc(-c4ccc5ccccc5c4)nc(-c4cccc5c4ccc4ccc6ccccc6c45)n3)ccc2c1 ; CC1(C)c2ccccc2-c2ccc(-c3nc(-c4ccccc4)nc(-c4cc5ccccc5c5c4ccc4ccccc45)n3)cc21 ; c1ccc(-c2nc(-c3ccc4c(ccc5ccccc54)c3)nc(-c3cc4ccccc4c4c3ccc3ccccc34)n2)cc1 ; c1ccc(-c2ccc(-c3nc(-c4ccc(-c5ccccc5)cc4)nc(-c4cc5ccccc5c5c4ccc4ccccc45)n3)cc2)cc1]

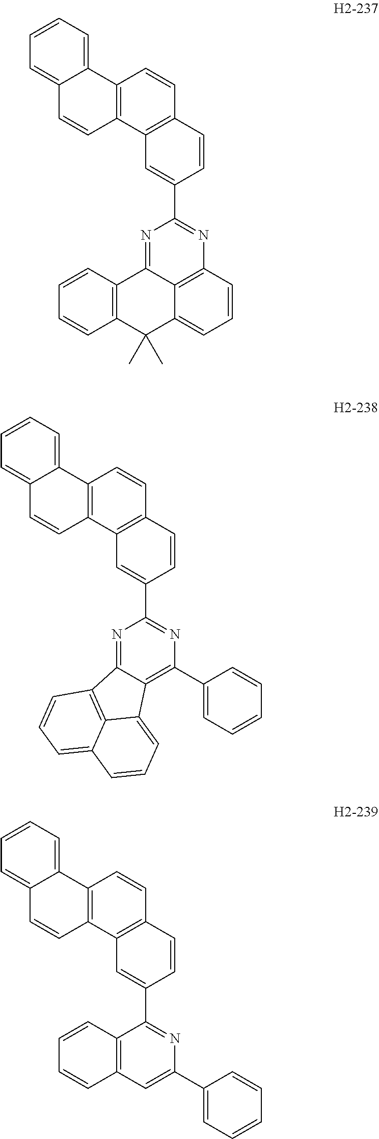 [SMILES: CC1(C)c2ccccc2-c2nc(-c3ccc4ccc5c6ccccc6ccc5c4c3)nc3cccc1c23 ; c1ccc(-c2nc(-c3ccc4ccc5c6ccccc6ccc5c4c3)nc3c2-c2cccc4cccc-3c24)cc1 ; c1ccc(-c2cc3ccccc3c(-c3ccc4ccc5c6ccccc6ccc5c4c3)n2)cc1]

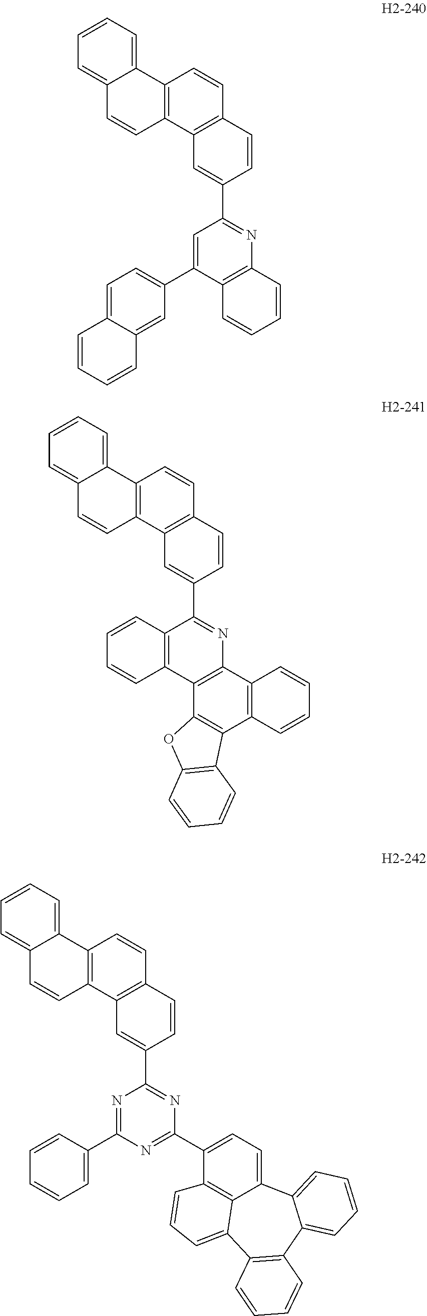

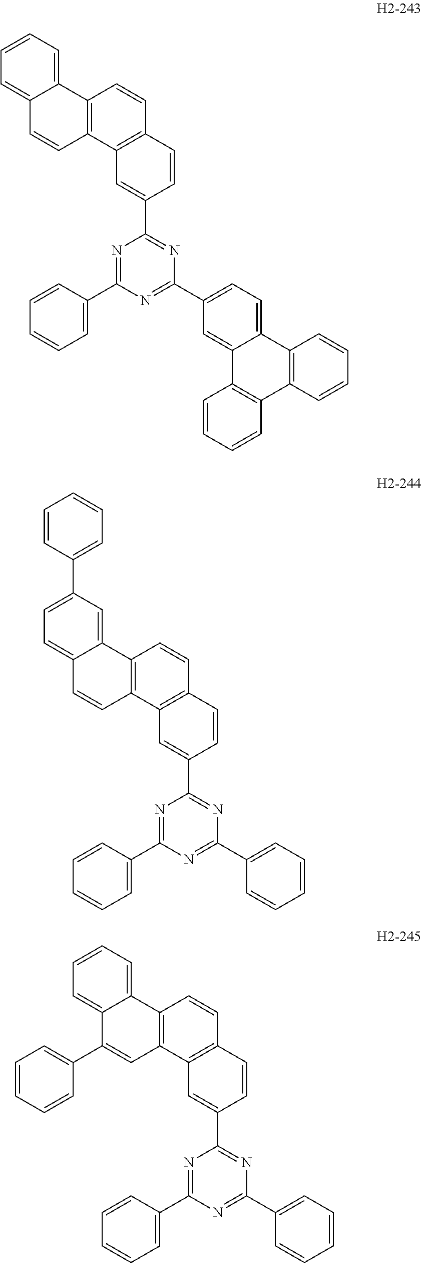

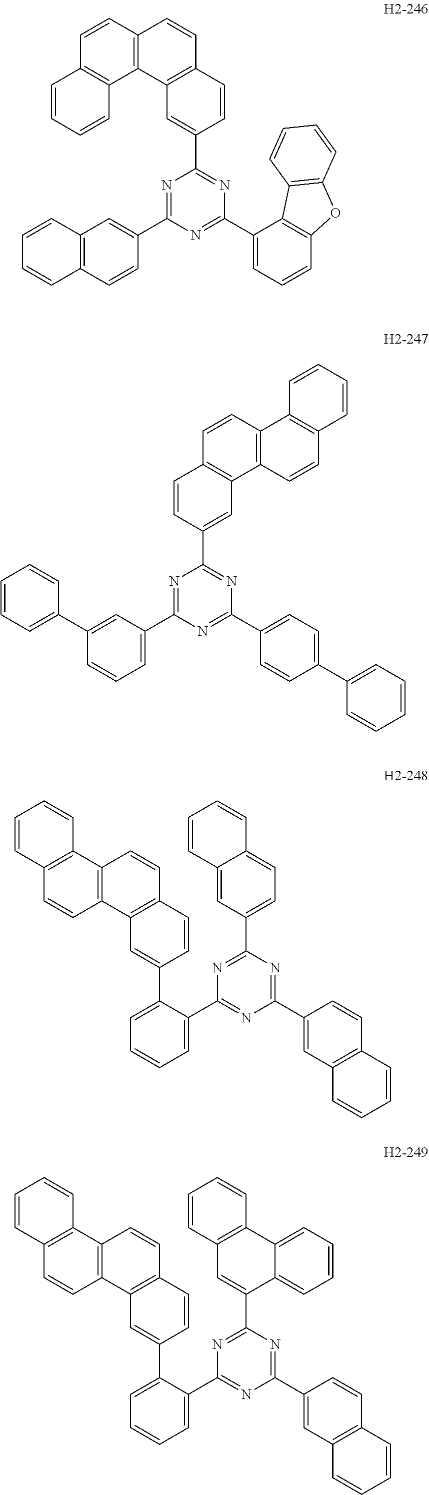 [SMILES: c1ccc2cc(-c3nc(-c4ccc5ccc6ccc7ccccc7c6c5c4)nc(-c4cccc5oc6ccccc6c45)n3)ccc2c1 ; c1ccc(-c2ccc(-c3nc(-c4cccc(-c5ccccc5)c4)nc(-c4ccc5ccc6c7ccccc7ccc6c5c4)n3)cc2)cc1 ; c1ccc(-c2nc(-c3ccc4ccccc4c3)nc(-c3ccc4ccccc4c3)n2)c(-c2ccc3ccc4c5ccccc5ccc4c3c2)c1 ; c1ccc(-c2nc(-c3ccc4ccccc4c3)nc(-c3cc4ccccc4c4ccccc34)n2)c(-c2ccc3ccc4c5ccccc5ccc4c3c2)c1]

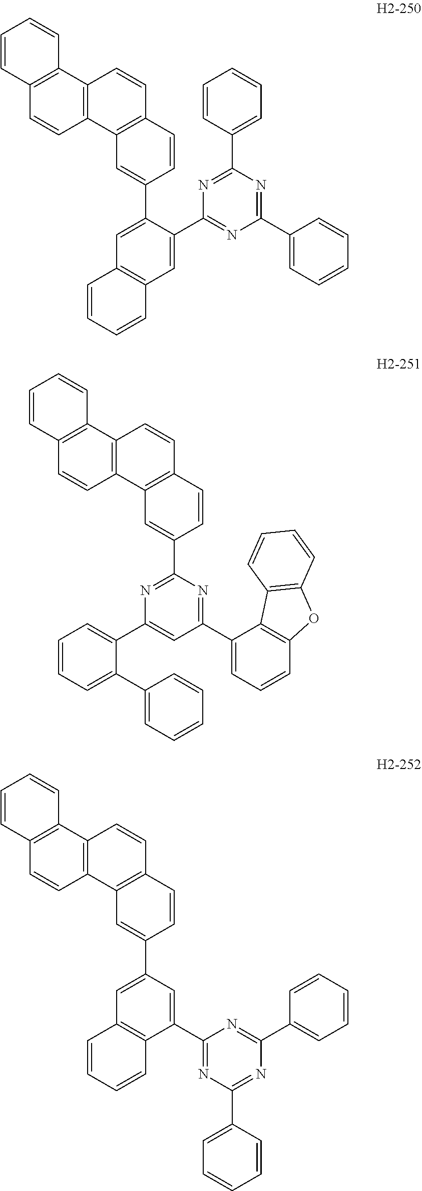 [SMILES: c1ccc(-c2nc(-c3ccccc3)nc(-c3cc4ccccc4cc3-c3ccc4ccc5c6ccccc6ccc5c4c3)n2)cc1 ; c1ccc(-c2ccccc2-c2cc(-c3cccc4oc5ccccc5c34)nc(-c3ccc4ccc5c6ccccc6ccc5c4c3)n2)cc1 ; c1ccc(-c2nc(-c3ccccc3)nc(-c3cc(-c4ccc5ccc6c7ccccc7ccc6c5c4)cc4ccccc34)n2)cc1]

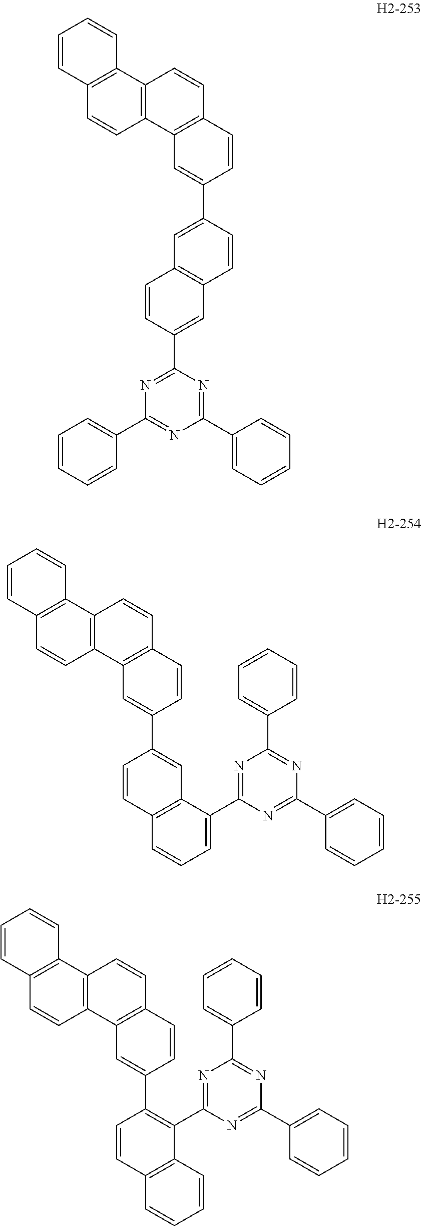 [SMILES: c1ccc(-c2nc(-c3ccccc3)nc(-c3ccc4cc(-c5ccc6ccc7c8ccccc8ccc7c6c5)ccc4c3)n2)cc1 ; c1ccc(-c2nc(-c3ccccc3)nc(-c3cccc4ccc(-c5ccc6ccc7c8ccccc8ccc7c6c5)cc34)n2)cc1 ; c1ccc(-c2nc(-c3ccccc3)nc(-c3c(-c4ccc5ccc6c7ccccc7ccc6c5c4)ccc4ccccc34)n2)cc1]

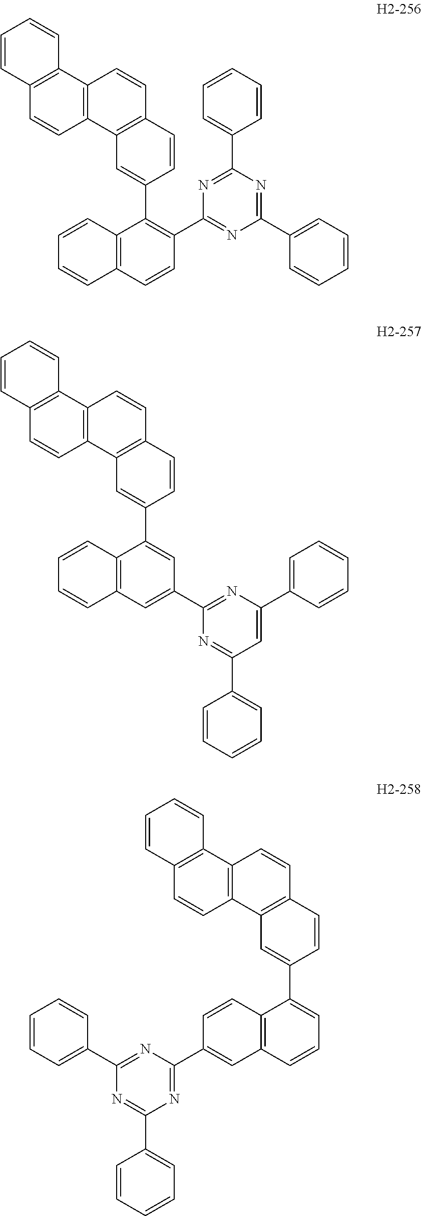 [SMILES: c1ccc(-c2nc(-c3ccccc3)nc(-c3ccc4ccccc4c3-c3ccc4ccc5c6ccccc6ccc5c4c3)n2)cc1 ; c1ccc(-c2cc(-c3ccccc3)nc(-c3cc(-c4ccc5ccc6c7ccccc7ccc6c5c4)c4ccccc4c3)n2)cc1 ; c1ccc(-c2nc(-c3ccccc3)nc(-c3ccc4c(-c5ccc6ccc7c8ccccc8ccc7c6c5)cccc4c3)n2)cc1]

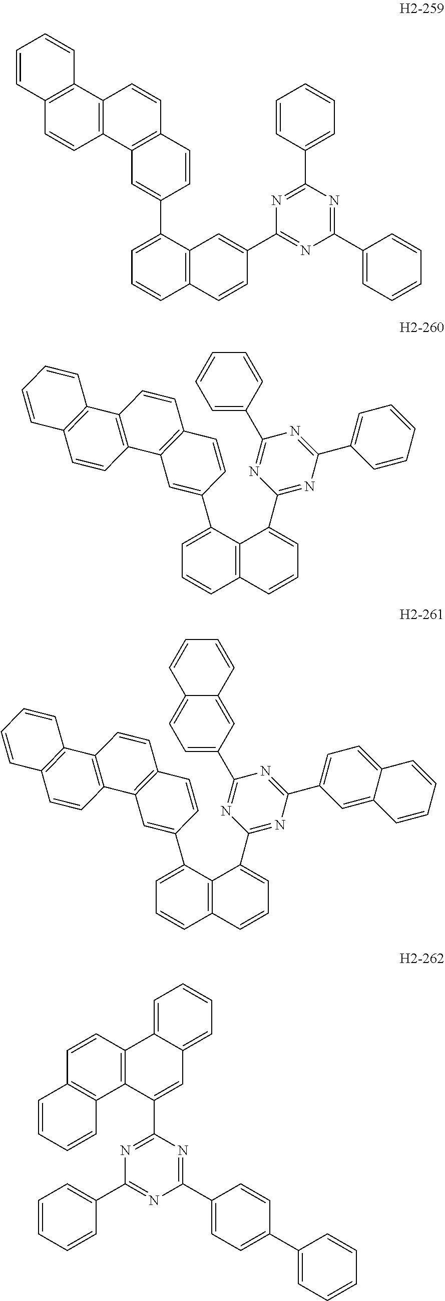

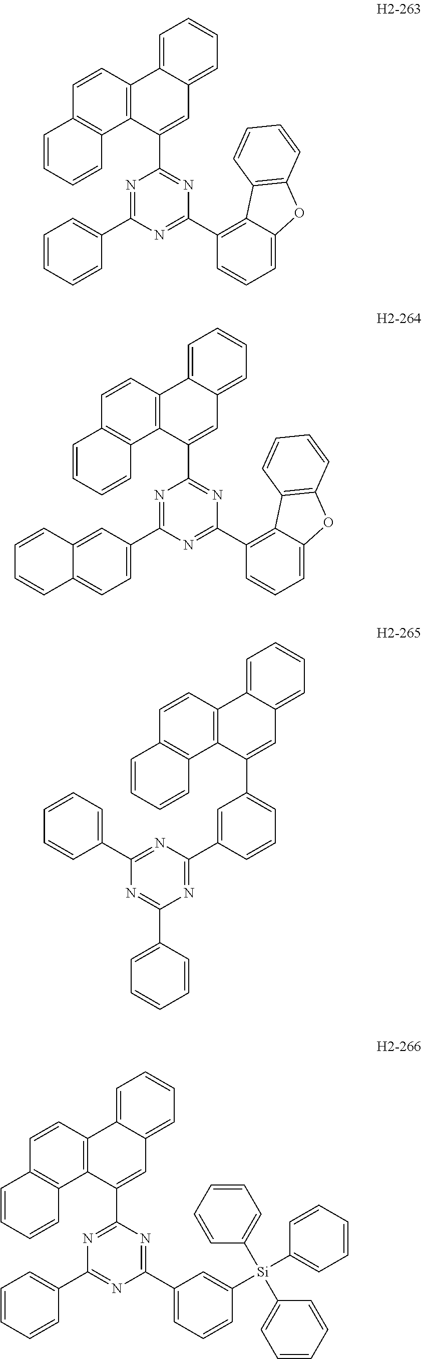 [SMILES: c1ccc(-c2nc(-c3cccc4oc5ccccc5c34)nc(-c3cc4ccccc4c4ccc5ccccc5c34)n2)cc1 ; c1ccc2cc(-c3nc(-c4cccc5oc6ccccc6c45)nc(-c4cc5ccccc5c5ccc6ccccc6c45)n3)ccc2c1 ; c1ccc(-c2nc(-c3ccccc3)nc(-c3cccc(-c4cc5ccccc5c5ccc6ccccc6c45)c3)n2)cc1 ; c1ccc(-c2nc(-c3cccc([Si](c4ccccc4)(c4ccccc4)c4ccccc4)c3)nc(-c3cc4ccccc4c4ccc5ccccc5c34)n2)cc1]

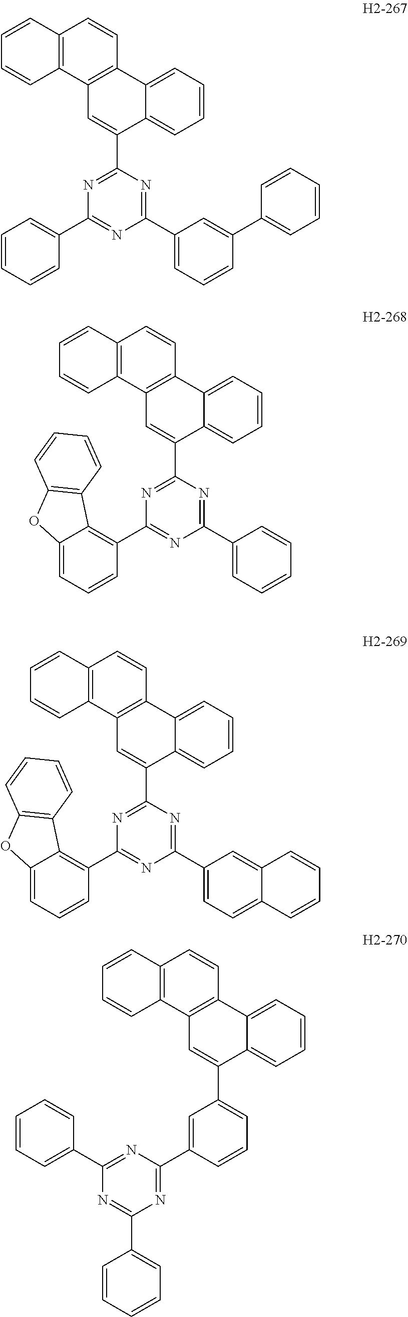 [SMILES: c1ccc(-c2cccc(-c3nc(-c4ccccc4)nc(-c4cc5c6ccccc6ccc5c5ccccc45)n3)c2)cc1 ; c1ccc(-c2nc(-c3cc4c5ccccc5ccc4c4ccccc34)nc(-c3cccc4oc5ccccc5c34)n2)cc1 ; c1ccc2cc(-c3nc(-c4cc5c6ccccc6ccc5c5ccccc45)nc(-c4cccc5oc6ccccc6c45)n3)ccc2c1 ; c1ccc(-c2nc(-c3ccccc3)nc(-c3cccc(-c4cc5c6ccccc6ccc5c5ccccc45)c3)n2)cc1]

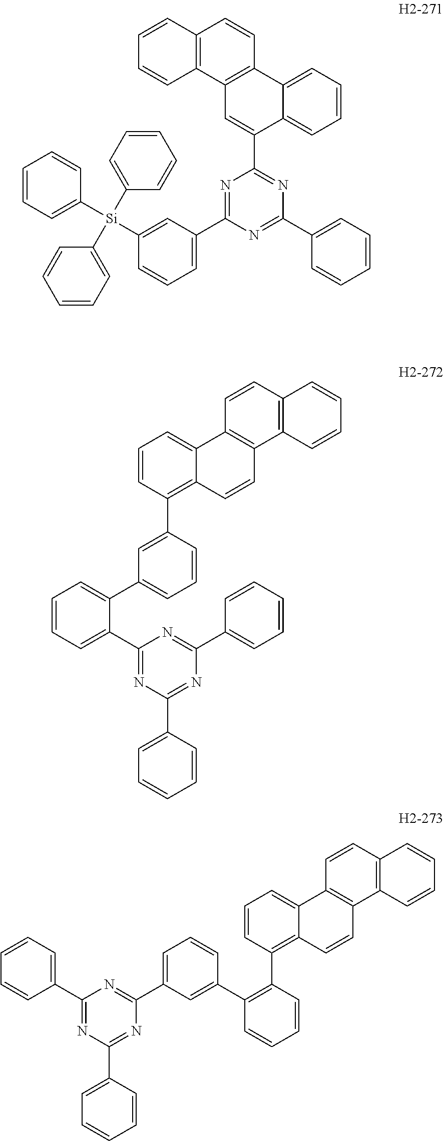 [SMILES: c1ccc(-c2nc(-c3cccc([Si](c4ccccc4)(c4ccccc4)c4ccccc4)c3)nc(-c3cc4c5ccccc5ccc4c4ccccc34)n2)cc1 ; c1ccc(-c2nc(-c3ccccc3)nc(-c3ccccc3-c3cccc(-c4cccc5c4ccc4c6ccccc6ccc54)c3)n2)cc1 ; c1ccc(-c2nc(-c3ccccc3)nc(-c3cccc(-c4ccccc4-c4cccc5c4ccc4c6ccccc6ccc54)c3)n2)cc1]

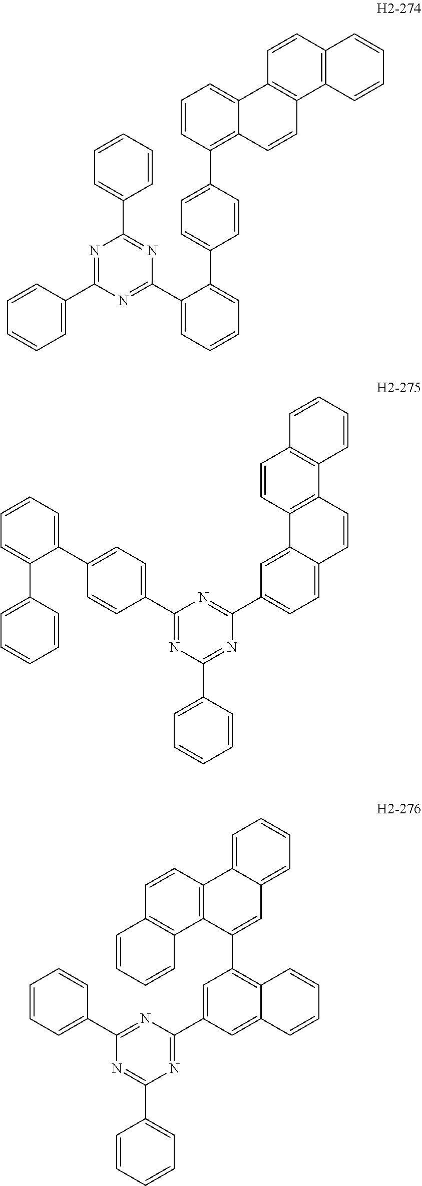 [SMILES: c1ccc(-c2nc(-c3ccccc3)nc(-c3ccccc3-c3ccc(-c4cccc5c4ccc4c6ccccc6ccc54)cc3)n2)cc1 ; c1ccc(-c2nc(-c3ccc(-c4ccccc4-c4ccccc4)cc3)nc(-c3ccc4ccc5c6ccccc6ccc5c4c3)n2)cc1 ; c1ccc(-c2nc(-c3ccccc3)nc(-c3cc(-c4cc5ccccc5c5ccc6ccccc6c45)c4ccccc4c3)n2)cc1]

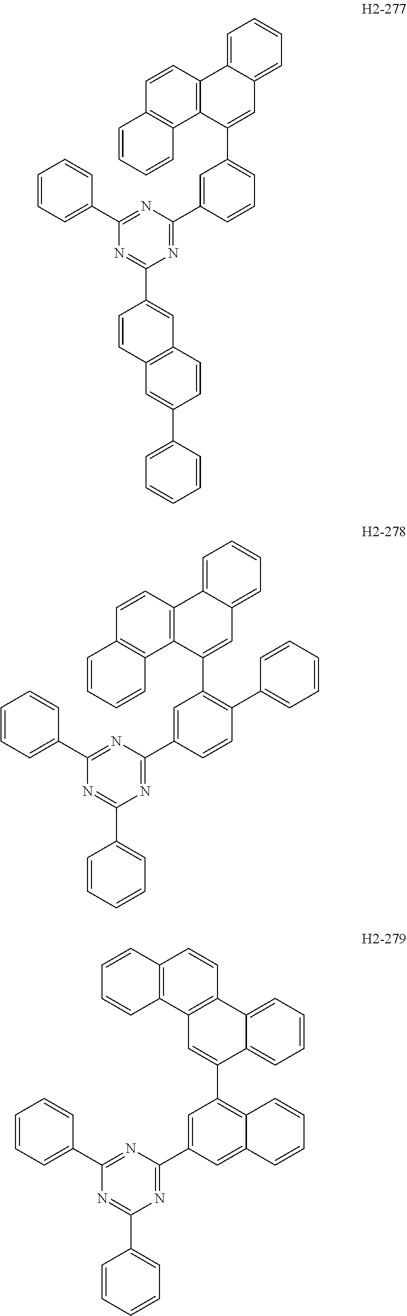

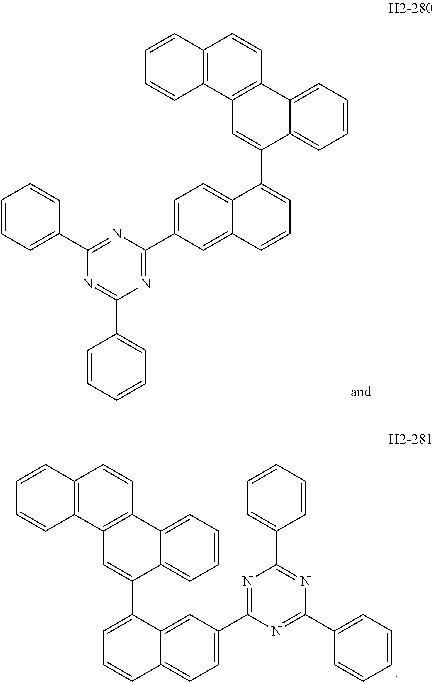 [SMILES: c1ccc(-c2nc(-c3ccccc3)nc(-c3ccc4c(-c5cc6c7ccccc7ccc6c6ccccc56)cccc4c3)n2)cc1 ; c1ccc(-c2nc(-c3ccccc3)nc(-c3ccc4cccc(-c5cc6c7ccccc7ccc6c6ccccc56)c4c3)n2)cc1]

9. An organic electroluminescent device comprising an anode, a cathode, and at least one light-emitting layer between the anode and the cathode, wherein at least one layer of the light-emitting layers comprises the plurality of host materials according to claim 1.

10. An organic electroluminescent compound represented by the following formula 2: 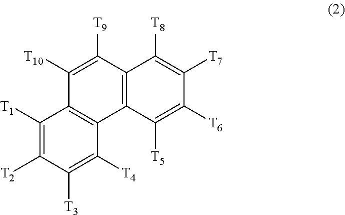
wherein
T₅ and T₆ are linked to each other to form a ring represented by the following formula 3; or T₇ and T₈ are linked to each other to form a ring represented by the following formula 3: 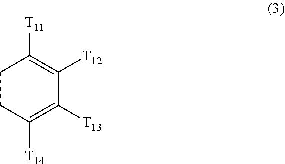
and in formulas 2 and 3,
T₁ to T₄, T₉ to T₁₄, and T₅ to T₈ not forming a ring, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arysilyl, a substituted or unsubstituted tri(C6-C30)arylsilyl, a substituted or unsubstituted fused ring group of a (C3-C30) aliphatic ring(s) and a (C6-C30) aromatic ring(s), a substituted or unsubstituted mono- or di-(C1-C30)alkylamino, a substituted or unsubstituted mono- or di-(C2-C30)alkenylamino, a substituted or unsubstituted (C1-C30)alkyl(C2-C30)alkenylamino, a substituted or unsubstituted mono- or di-(C6-C30)arylamino, a substituted or unsubstituted (C1-C30)alkyl(C6-C30)arylamino, a substituted or unsubstituted mono- or di-(3- to 30-membered)heteroarylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, a substituted or unsubstituted (C6-C30)aryl(3- to 30-membered)heteroarylamino, or -L₂-Ar₂; with the proviso that at least one of T₁ to T₄, T₁₁ to T₁₄, and T₅ to T₈ not forming a ring represents -L₂-Ar₂;
L₂, each independently, represents a single bond, a substituted or unsubstituted (C6-C30)arylene, or a substituted or unsubstituted (3- to 30-membered)heteroarylene;
Ar₂, each independently, represents a substituted or unsubstituted (C6-C30)aryl, or a substituted or unsubstituted (3- to 30-membered)heteroaryl; [symbol]
represents a fusion site with formula 2; and
the heteroaryl contains at least one heteroatom selected from the group consisting of B, N, O, S, Si, and P.

11. The organic electroluminescent compound according to claim 10, wherein the formula 2 is represented by at least one of the following formulas 2-1 and 2-2: 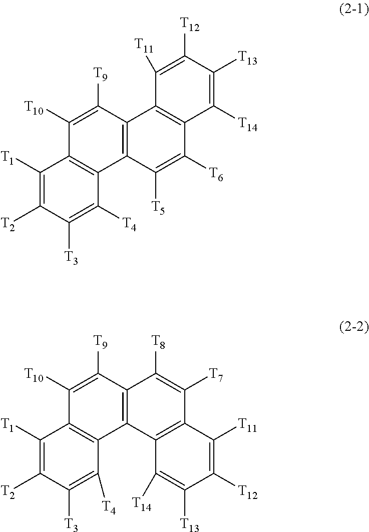
wherein,
T₁ to T₄, and T₉ to T₁₄ are as defined in claim 10, and T₅ to T₆ are defined as T₅ to T₈ not forming a ring in claim 10.

12. The organic electroluminescent compound according to claim 10, wherein the formula 2 is represented by at least one of the following formulas: 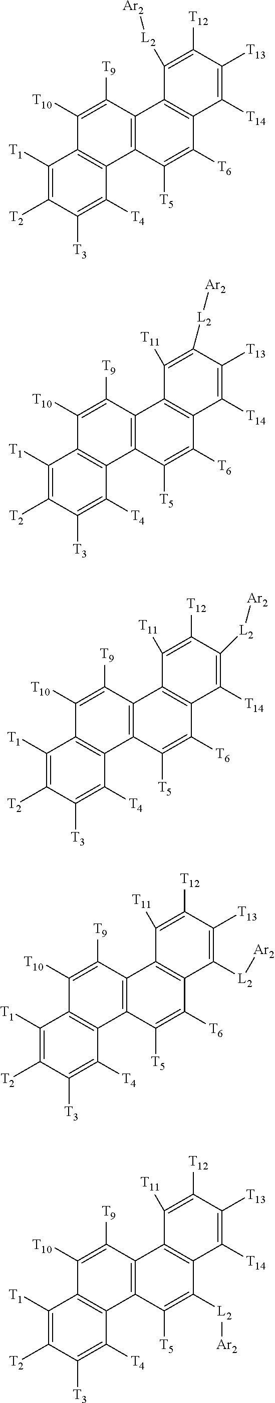 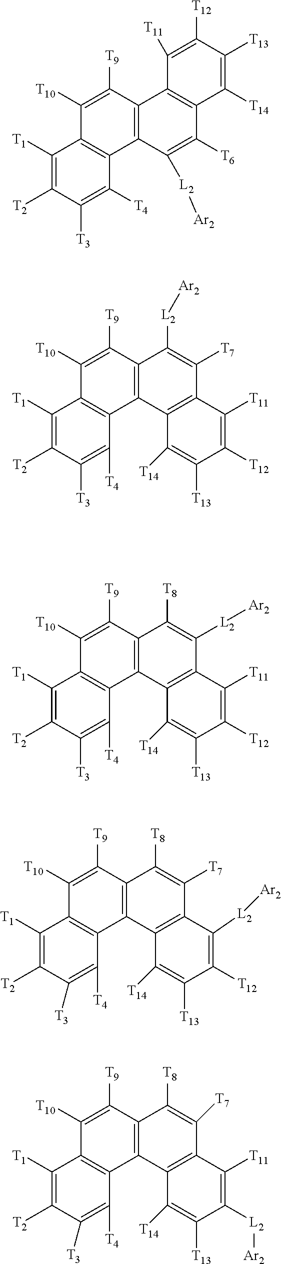 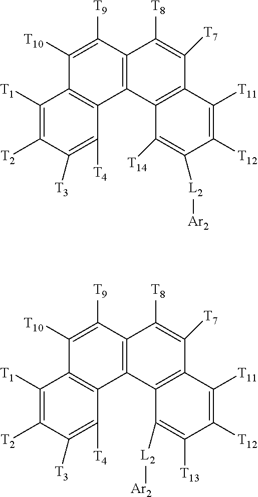
wherein,
T₁ to T₁₄, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring group of a (C3-C30) aliphatic ring(s) and a (C6-C30) aromatic ring(s), a substituted or unsubstituted mono- or di-(C1-C30)alkylamino, a substituted or unsubstituted mono- or di-(C2-C30)alkenylamino, a substituted or unsubstituted (C1-C30)alkyl(C2-C30)alkenylamino, a substituted or unsubstituted mono- or di-(C6-C30)arylamino, a substituted or unsubstituted (C1-C30)alkyl(C6-C30)arylamino, a substituted or unsubstituted mono- or di-(3- to 30-membered)heteroarylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, or a substituted or unsubstituted (C6-C30)aryl(3- to 30-membered)heteroarylamino; and
L₂ and Ar₂ are as defined in claim 10.

13. The organic electroluminescent compound according to claim 10, wherein the arylene of L₂ represents a 1,2-phenylene, a 1,3-phenylene, a 1,4-phenylene, a 1,2-biphenylene, a 1,3-biphenylene, a 1,4-biphenylene, a 1,2-terphenylene, a 1,3-terphenylene, a 1,4-terphenylene, a 1,2-naphthylene, a 1,3-naphthylene, a 1,4-naphthylene, a 1,5-naphthylene, a 1,6-naphthylene, a 1,7-naphthylene, a 1,8-naphthylene, a 2,3-naphthylene, a 2,6-naphthylene, a 2,7-naphthylene, or a phenanthrenylene.

14. The organic electroluminescent compound according to claim 10, wherein the heteroaryl of Ar₂ represents a pyridyl, a pyrimidinyl, a triazinyl, a quinazolinyl, a benzoquinazolinyl, a dibenzoquinazolinyl, a quinoxalinyl, a benzoquinoxalinyl, a dibenzoquinoxalinyl, a quinolyl, a benzoquinolyl, an isoquinolyl, a naphthyridinyl, a benzoisoquinolyl, an imidazolyl, a benzimidazolyl, a phenanthroimidazolyl, a thiazolyl, a benzothiazolyl, a phenanthrothiazolyl, an oxazolyl, a benzoxazolyl, a phenanthro oxazolyl, a naphtho oxazolyl, a naphthothiazolyl, a benzothienopyrimidinyl, a benzothienopyrazinyl, a benzofuropyrimidinyl, a benzofuropyrazinyl, a benzothienoquinolyl, a benzofuroquinolyl, an acenaphthopyrimidinyl, a benzobenzofurophenanthridinyl, a carbazolyl, a dibenzofuranyl, a dibenzothiophenyl, a benzonaphthofuranyl, a benzonaphthothiophenyl, a benzocarbazolyl, a dibenzocarbazolyl, a phenoxazinyl, or a benzoperimidinyl.

15. The organic electroluminescent compound according to claim 10, wherein the compound represented by formula 2 is at least one selected from the following compounds:

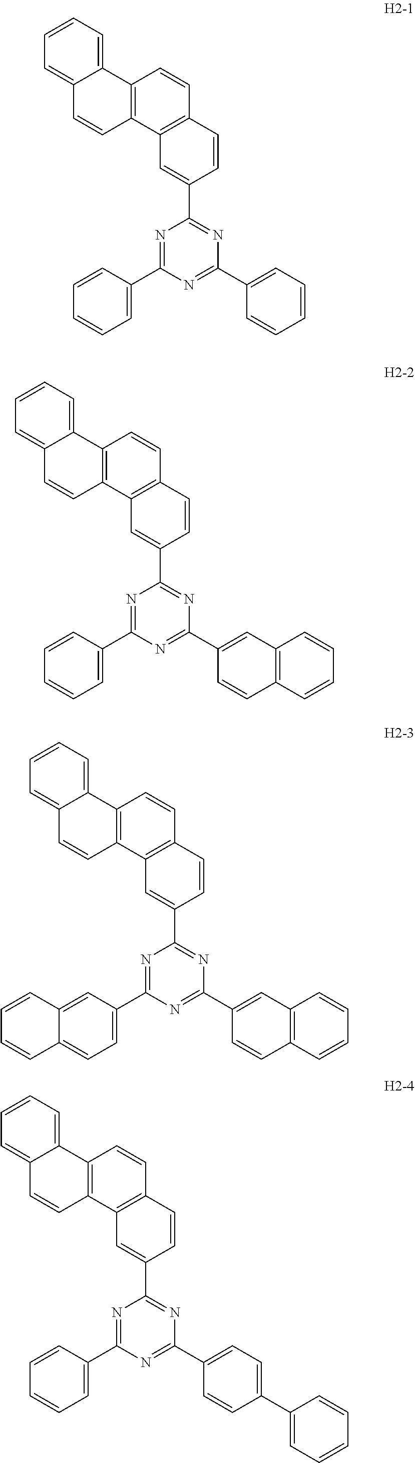

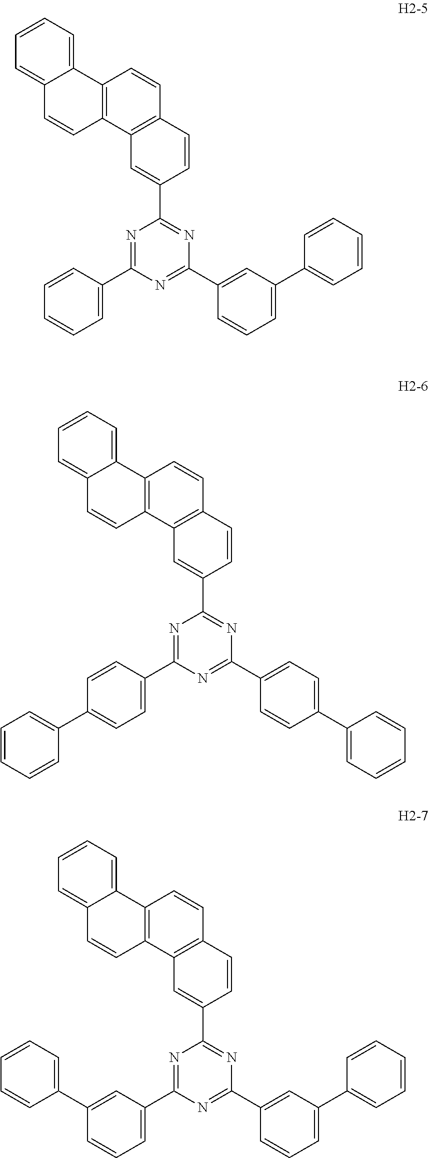 [SMILES: c1ccc(-c2cccc(-c3nc(-c4ccccc4)nc(-c4ccc5ccc6c7ccccc7ccc6c5c4)n3)c2)cc1 ; c1ccc(-c2ccc(-c3nc(-c4ccc(-c5ccccc5)cc4)nc(-c4ccc5ccc6c7ccccc7ccc6c5c4)n3)cc2)cc1 ; c1ccc(-c2cccc(-c3nc(-c4cccc(-c5ccccc5)c4)nc(-c4ccc5ccc6c7ccccc7ccc6c5c4)n3)c2)cc1]

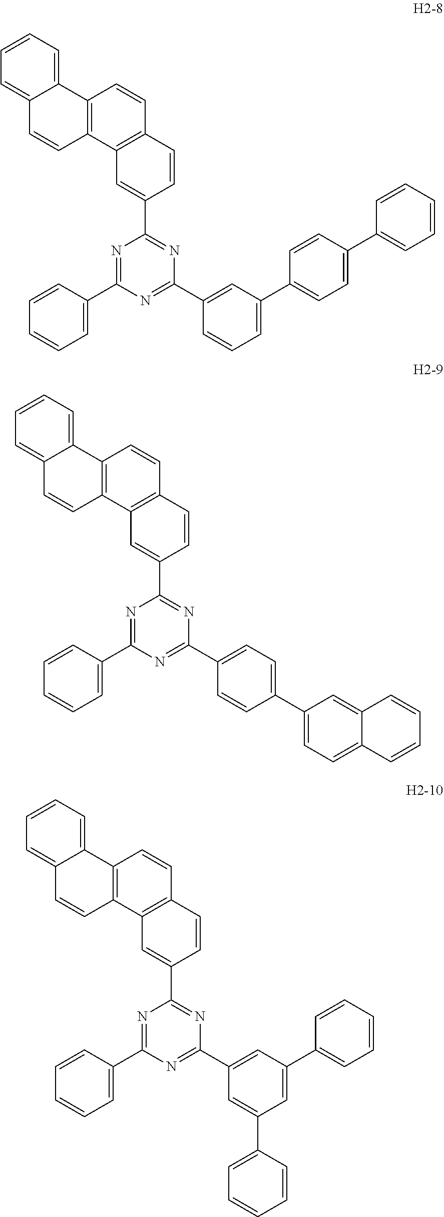

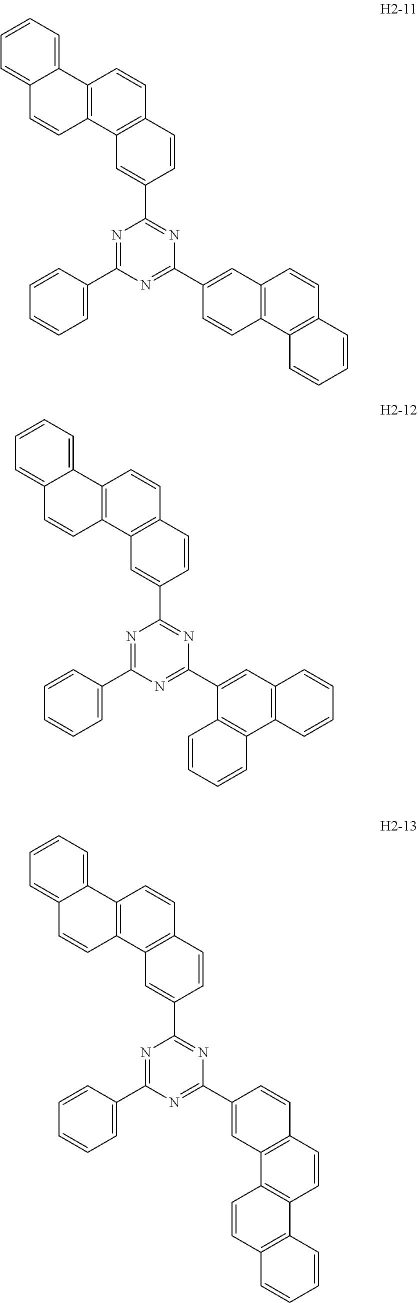 [SMILES: c1ccc(-c2nc(-c3ccc4c(ccc5ccccc54)c3)nc(-c3ccc4ccc5c6ccccc6ccc5c4c3)n2)cc1 ; c1ccc(-c2nc(-c3ccc4ccc5c6ccccc6ccc5c4c3)nc(-c3cc4ccccc4c4ccccc34)n2)cc1 ; c1ccc(-c2nc(-c3ccc4ccc5c6ccccc6ccc5c4c3)nc(-c3ccc4ccc5c6ccccc6ccc5c4c3)n2)cc1]

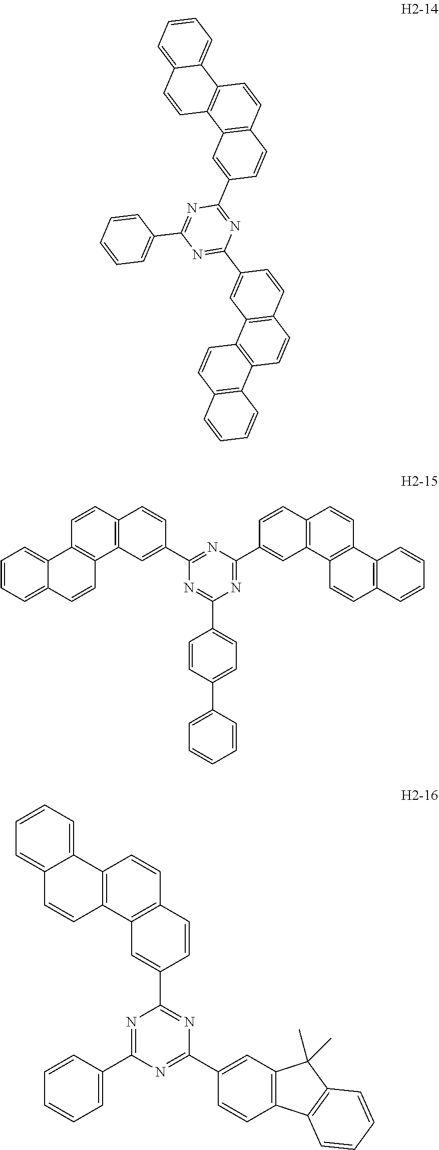 [SMILES: c1ccc(-c2nc(-c3ccc4ccc5c6ccccc6ccc5c4c3)nc(-c3ccc4ccc5c6ccccc6ccc5c4c3)n2)cc1 ; c1ccc(-c2ccc(-c3nc(-c4ccc5ccc6c7ccccc7ccc6c5c4)nc(-c4ccc5ccc6c7ccccc7ccc6c5c4)n3)cc2)cc1 ; CC1(C)c2ccccc2-c2ccc(-c3nc(-c4ccccc4)nc(-c4ccc5ccc6c7ccccc7ccc6c5c4)n3)cc21]

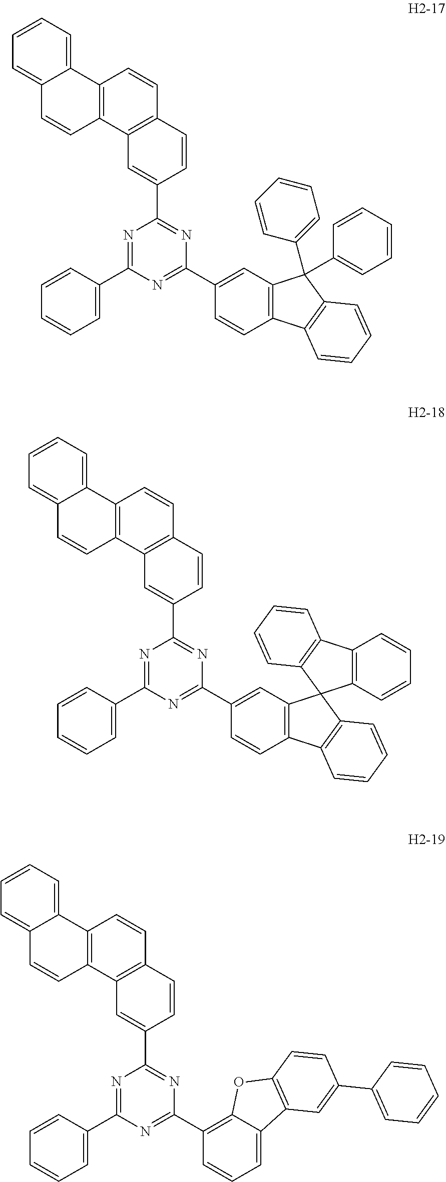 [SMILES: c1ccc(-c2nc(-c3ccc4c(c3)C(c3ccccc3)(c3ccccc3)c3ccccc3-4)nc(-c3ccc4ccc5c6ccccc6ccc5c4c3)n2)cc1 ; c1ccc(-c2nc(-c3ccc4c(c3)C3(c5ccccc5-c5ccccc53)c3ccccc3-4)nc(-c3ccc4ccc5c6ccccc6ccc5c4c3)n2)cc1 ; c1ccc(-c2ccc3oc4c(-c5nc(-c6ccccc6)nc(-c6ccc7ccc8c9ccccc9ccc8c7c6)n5)cccc4c3c2)cc1]

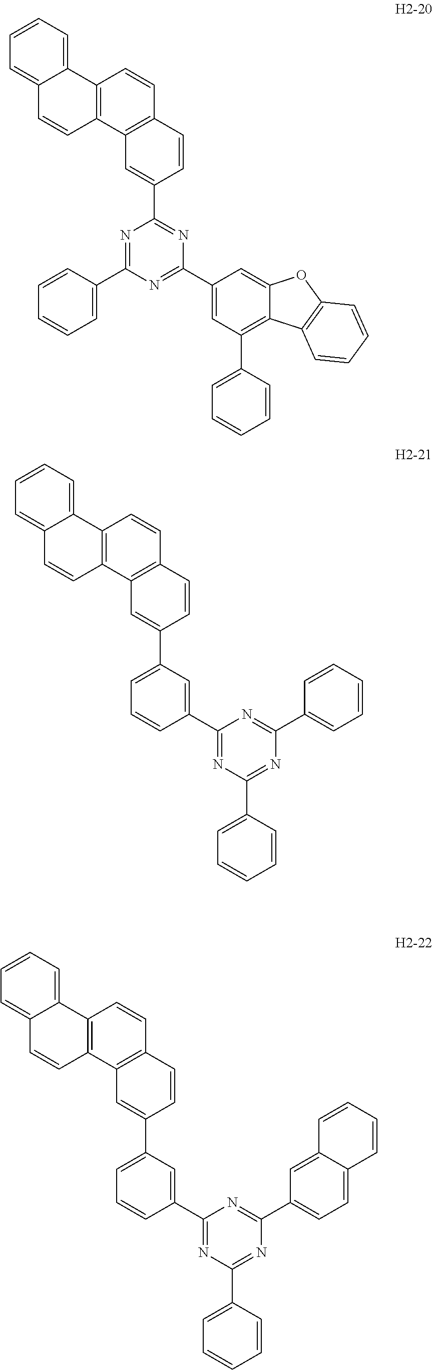 [SMILES: c1ccc(-c2nc(-c3cc(-c4ccccc4)c4c(c3)oc3ccccc34)nc(-c3ccc4ccc5c6ccccc6ccc5c4c3)n2)cc1 ; c1ccc(-c2nc(-c3ccccc3)nc(-c3cccc(-c4ccc5ccc6c7ccccc7ccc6c5c4)c3)n2)cc1 ; c1ccc(-c2nc(-c3cccc(-c4ccc5ccc6c7ccccc7ccc6c5c4)c3)nc(-c3ccc4ccccc4c3)n2)cc1]

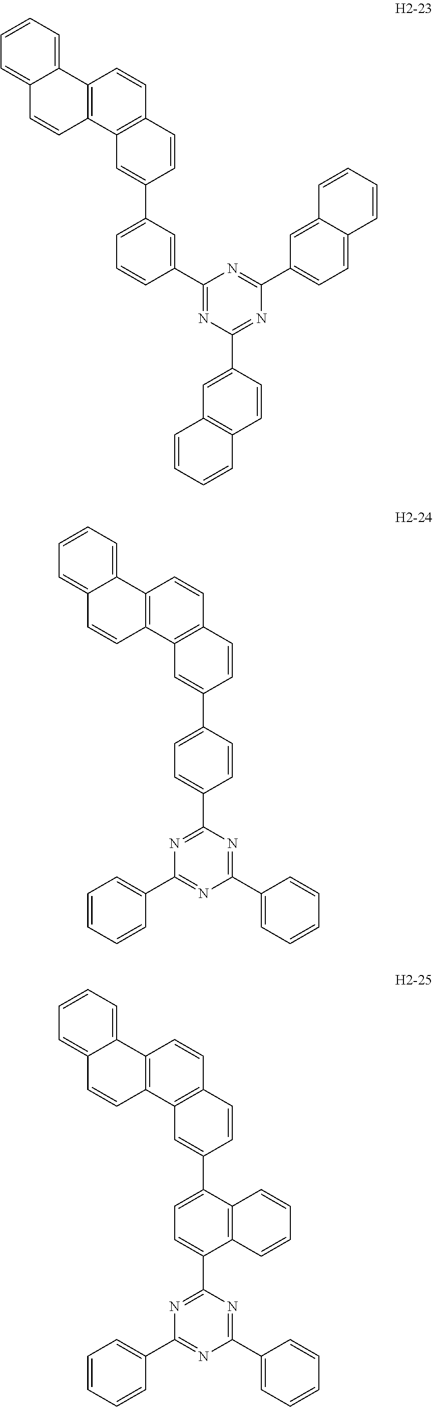 [SMILES: c1cc(-c2ccc3ccc4c5ccccc5ccc4c3c2)cc(-c2nc(-c3ccc4ccccc4c3)nc(-c3ccc4ccccc4c3)n2)c1 ; c1ccc(-c2nc(-c3ccccc3)nc(-c3ccc(-c4ccc5ccc6c7ccccc7ccc6c5c4)cc3)n2)cc1 ; c1ccc(-c2nc(-c3ccccc3)nc(-c3ccc(-c4ccc5ccc6c7ccccc7ccc6c5c4)c4ccccc34)n2)cc1]

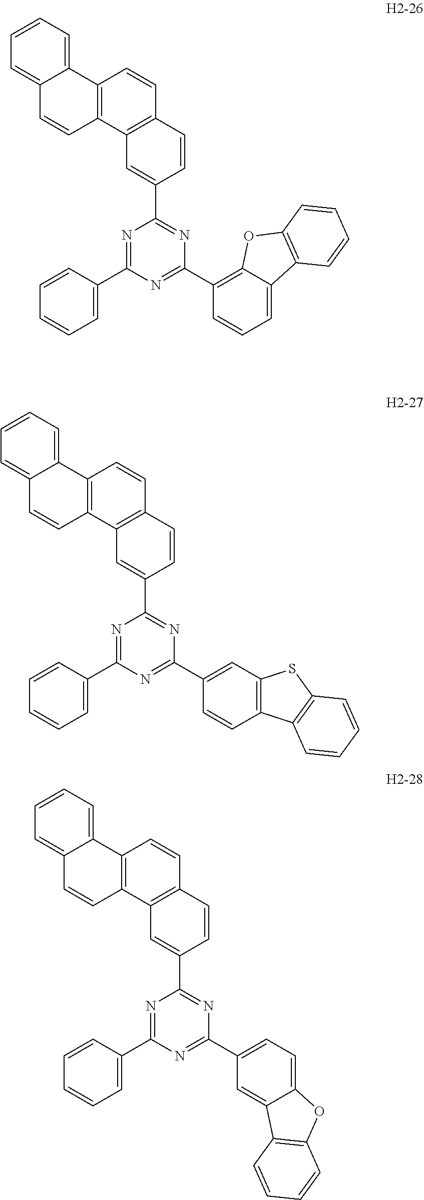 [SMILES: c1ccc(-c2nc(-c3ccc4ccc5c6ccccc6ccc5c4c3)nc(-c3cccc4c3oc3ccccc34)n2)cc1 ; c1ccc(-c2nc(-c3ccc4c(c3)sc3ccccc34)nc(-c3ccc4ccc5c6ccccc6ccc5c4c3)n2)cc1 ; c1ccc(-c2nc(-c3ccc4ccc5c6ccccc6ccc5c4c3)nc(-c3ccc4oc5ccccc5c4c3)n2)cc1]

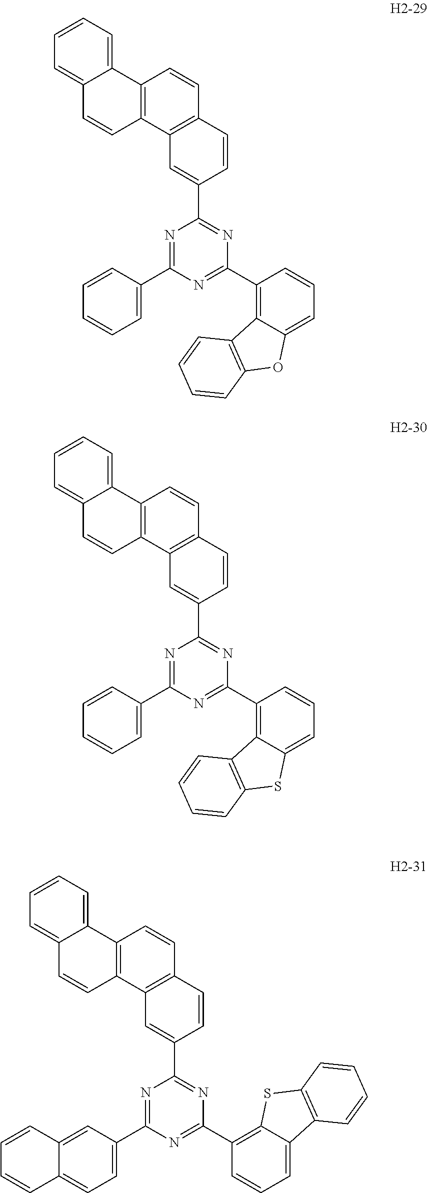 [SMILES: c1ccc(-c2nc(-c3ccc4ccc5c6ccccc6ccc5c4c3)nc(-c3cccc4oc5ccccc5c34)n2)cc1 ; c1ccc(-c2nc(-c3ccc4ccc5c6ccccc6ccc5c4c3)nc(-c3cccc4sc5ccccc5c34)n2)cc1 ; c1ccc2cc(-c3nc(-c4ccc5ccc6c7ccccc7ccc6c5c4)nc(-c4cccc5c4sc4ccccc45)n3)ccc2c1]

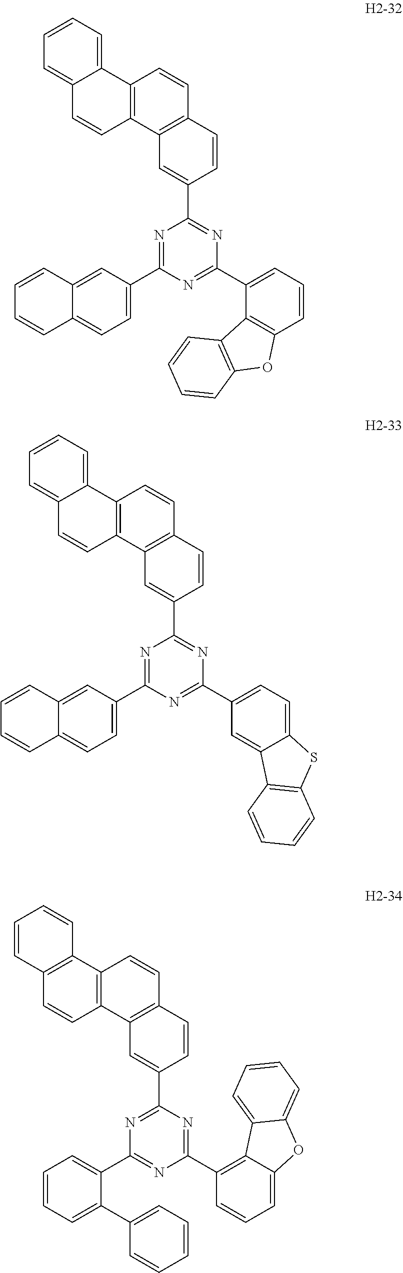 [SMILES: c1ccc2cc(-c3nc(-c4ccc5ccc6c7ccccc7ccc6c5c4)nc(-c4cccc5oc6ccccc6c45)n3)ccc2c1 ; c1ccc2cc(-c3nc(-c4ccc5ccc6c7ccccc7ccc6c5c4)nc(-c4ccc5sc6ccccc6c5c4)n3)ccc2c1 ; c1ccc(-c2ccccc2-c2nc(-c3ccc4ccc5c6ccccc6ccc5c4c3)nc(-c3cccc4oc5ccccc5c34)n2)cc1]

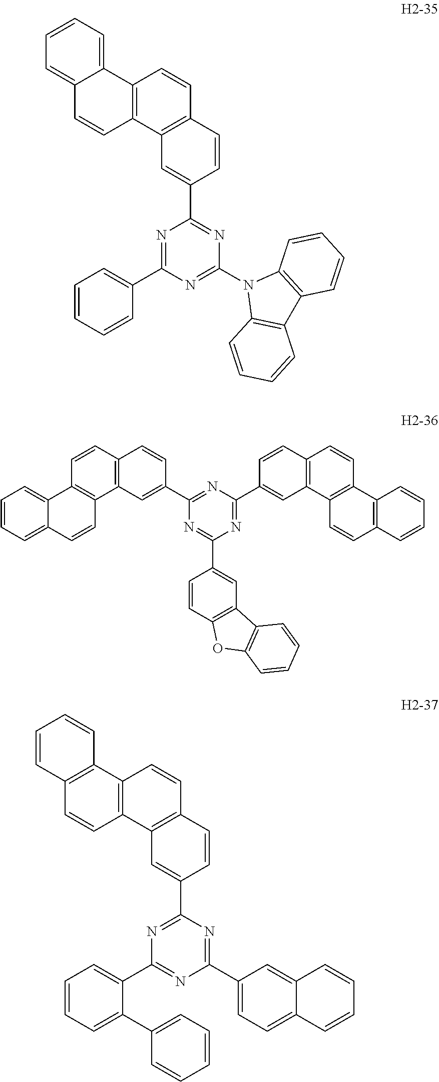 [SMILES: c1ccc(-c2nc(-c3ccc4ccc5c6ccccc6ccc5c4c3)nc(-n3c4ccccc4c4ccccc43)n2)cc1 ; c1ccc2c(c1)ccc1c3cc(-c4nc(-c5ccc6ccc7c8ccccc8ccc7c6c5)nc(-c5ccc6oc7ccccc7c6c5)n4)ccc3ccc21 ; c1ccc(-c2ccccc2-c2nc(-c3ccc4ccccc4c3)nc(-c3ccc4ccc5c6ccccc6ccc5c4c3)n2)cc1]

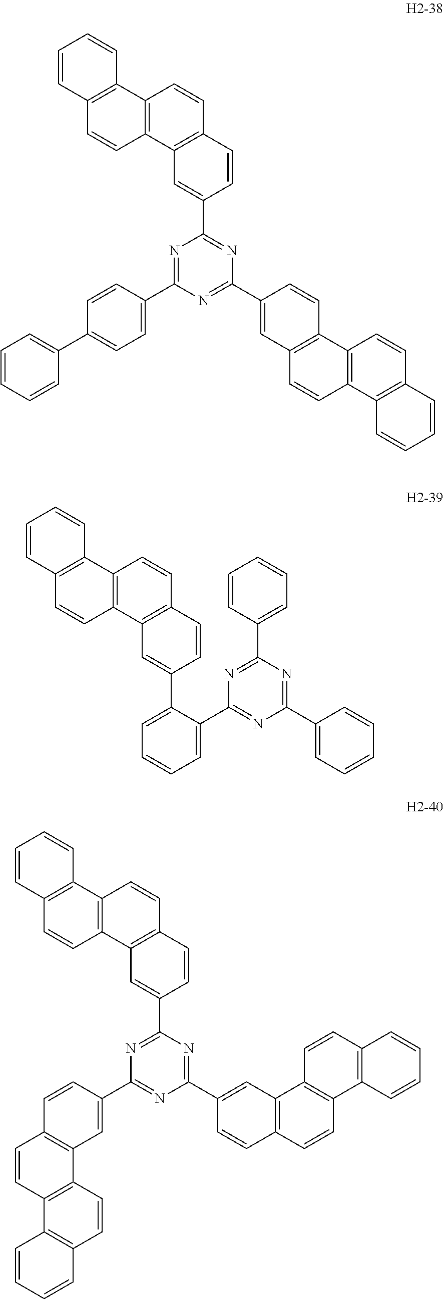 [SMILES: c1ccc(-c2ccc(-c3nc(-c4ccc5c(ccc6c7ccccc7ccc56)c4)nc(-c4ccc5ccc6c7ccccc7ccc6c5c4)n3)cc2)cc1 ; c1ccc(-c2nc(-c3ccccc3)nc(-c3ccccc3-c3ccc4ccc5c6ccccc6ccc5c4c3)n2)cc1 ; c1ccc2c(c1)ccc1c3cc(-c4nc(-c5ccc6ccc7c8ccccc8ccc7c6c5)nc(-c5ccc6ccc7c8ccccc8ccc7c6c5)n4)ccc3ccc21]

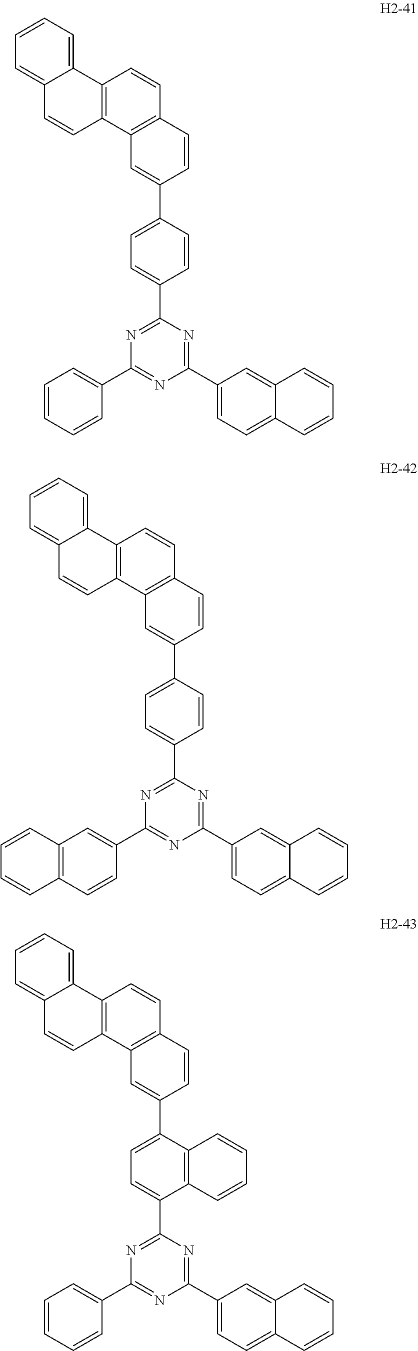

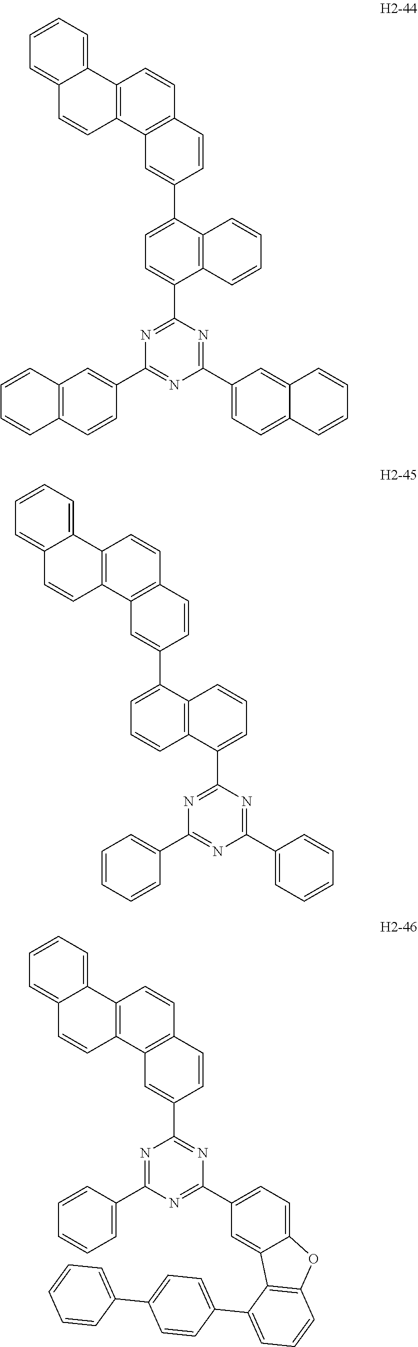 [SMILES: c1ccc2cc(-c3nc(-c4ccc5ccccc5c4)nc(-c4ccc(-c5ccc6ccc7c8ccccc8ccc7c6c5)c5ccccc45)n3)ccc2c1 ; c1ccc(-c2nc(-c3ccccc3)nc(-c3cccc4c(-c5ccc6ccc7c8ccccc8ccc7c6c5)cccc34)n2)cc1 ; c1ccc(-c2ccc(-c3cccc4oc5ccc(-c6nc(-c7ccccc7)nc(-c7ccc8ccc9c%10ccccc%10ccc9c8c7)n6)cc5c34)cc2)cc1]

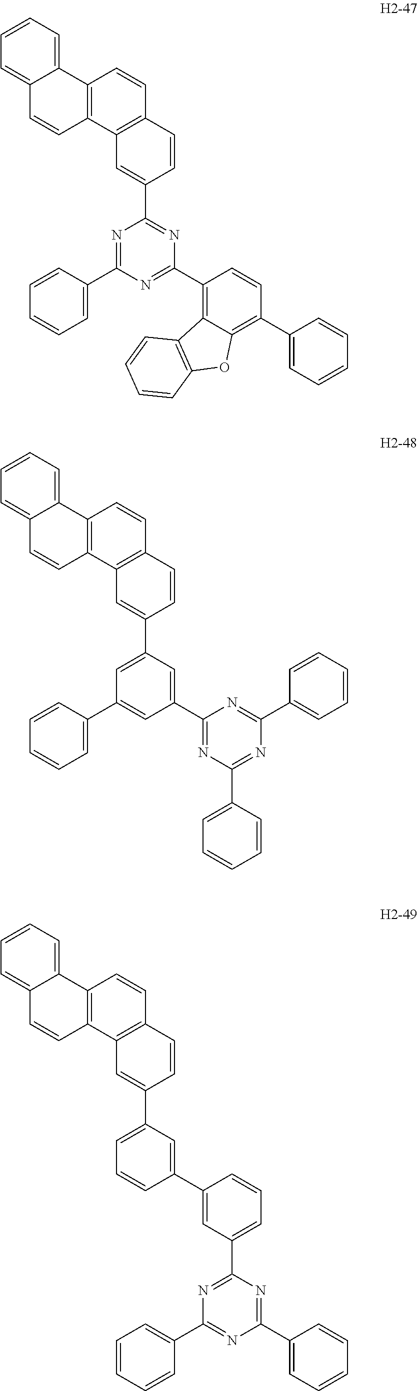 [SMILES: c1ccc(-c2nc(-c3ccc4ccc5c6ccccc6ccc5c4c3)nc(-c3ccc(-c4ccccc4)c4oc5ccccc5c34)n2)cc1 ; c1ccc(-c2cc(-c3ccc4ccc5c6ccccc6ccc5c4c3)cc(-c3nc(-c4ccccc4)nc(-c4ccccc4)n3)c2)cc1 ; c1ccc(-c2nc(-c3ccccc3)nc(-c3cccc(-c4cccc(-c5ccc6ccc7c8ccccc8ccc7c6c5)c4)c3)n2)cc1]

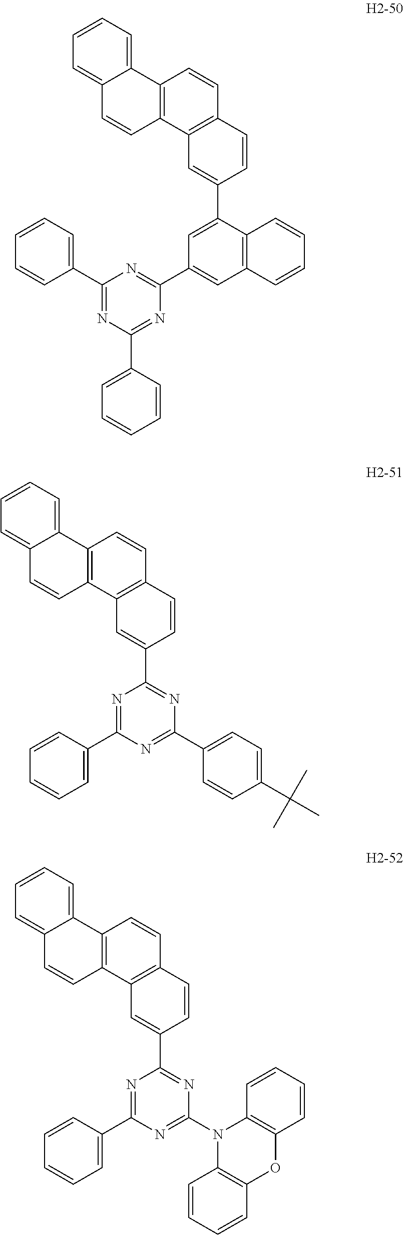 [SMILES: c1ccc(-c2nc(-c3ccccc3)nc(-c3cc(-c4ccc5ccc6c7ccccc7ccc6c5c4)c4ccccc4c3)n2)cc1 ; CC(C)(C)c1ccc(-c2nc(-c3ccccc3)nc(-c3ccc4ccc5c6ccccc6ccc5c4c3)n2)cc1 ; c1ccc(-c2nc(-c3ccc4ccc5c6ccccc6ccc5c4c3)nc(N3c4ccccc4Oc4ccccc43)n2)cc1]

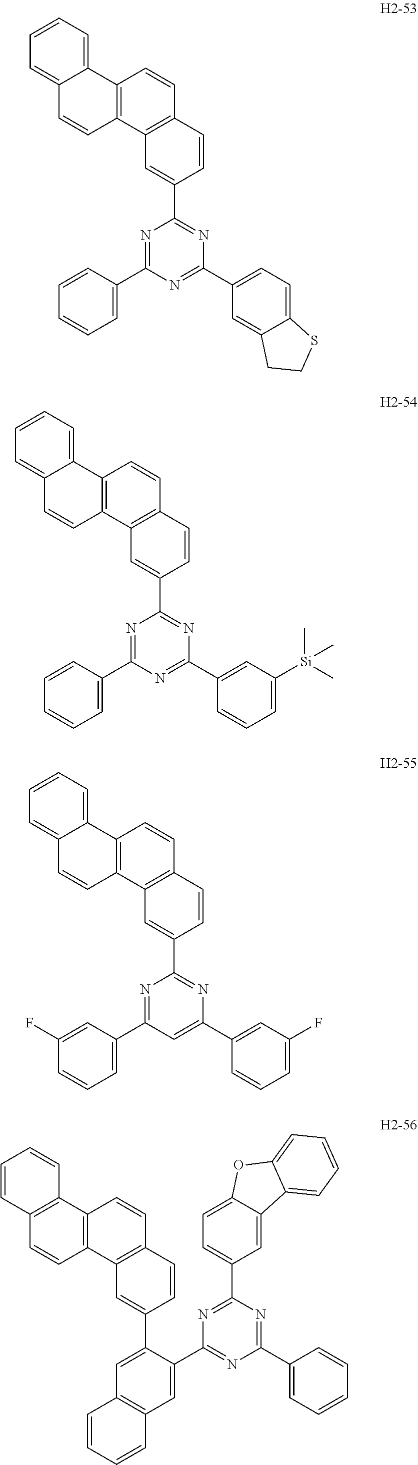 [SMILES: c1ccc(-c2nc(-c3ccc4c(c3)CCS4)nc(-c3ccc4ccc5c6ccccc6ccc5c4c3)n2)cc1 ; C[Si](C)(C)c1cccc(-c2nc(-c3ccccc3)nc(-c3ccc4ccc5c6ccccc6ccc5c4c3)n2)c1 ; Fc1cccc(-c2cc(-c3cccc(F)c3)nc(-c3ccc4ccc5c6ccccc6ccc5c4c3)n2)c1 ; c1ccc(-c2nc(-c3ccc4oc5ccccc5c4c3)nc(-c3cc4ccccc4cc3-c3ccc4ccc5c6ccccc6ccc5c4c3)n2)cc1]

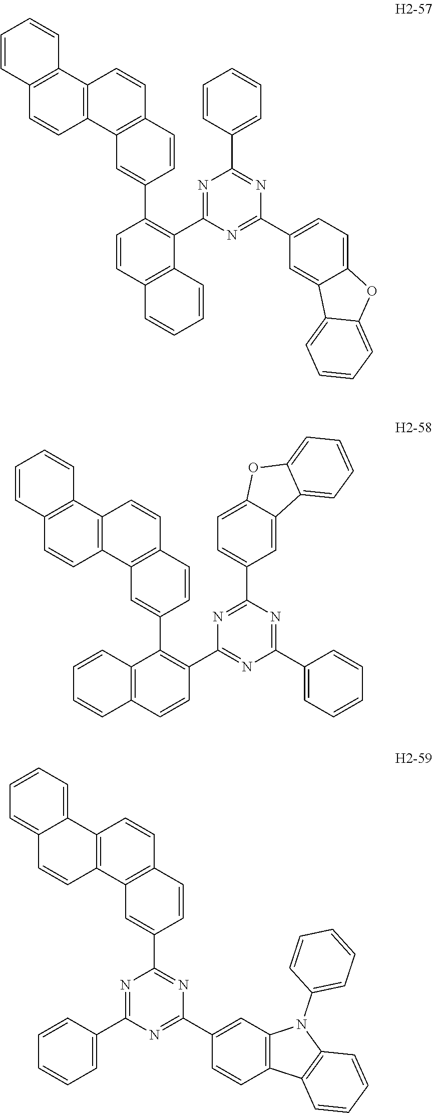 [SMILES: c1ccc(-c2nc(-c3ccc4oc5ccccc5c4c3)nc(-c3c(-c4ccc5ccc6c7ccccc7ccc6c5c4)ccc4ccccc34)n2)cc1 ; c1ccc(-c2nc(-c3ccc4oc5ccccc5c4c3)nc(-c3ccc4ccccc4c3-c3ccc4ccc5c6ccccc6ccc5c4c3)n2)cc1 ; c1ccc(-c2nc(-c3ccc4ccc5c6ccccc6ccc5c4c3)nc(-c3ccc4c5ccccc5n(-c5ccccc5)c4c3)n2)cc1]

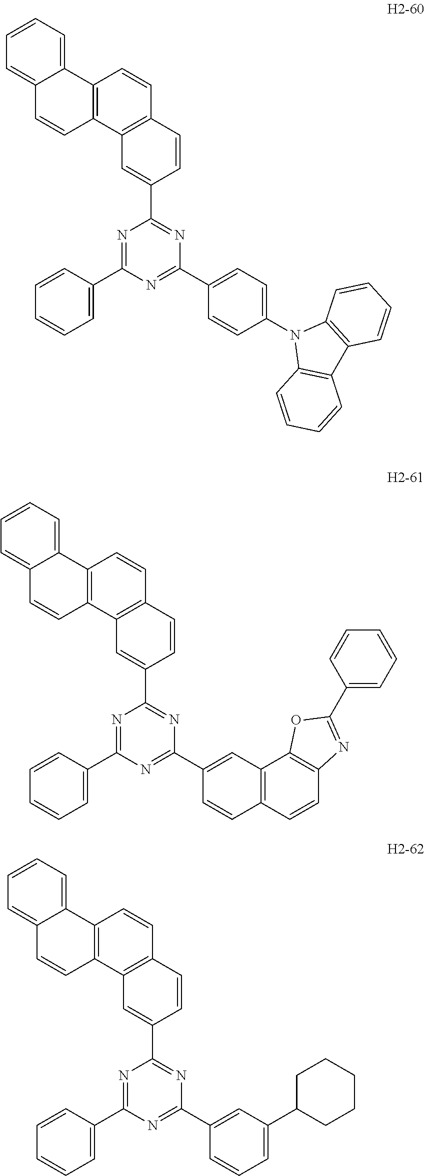

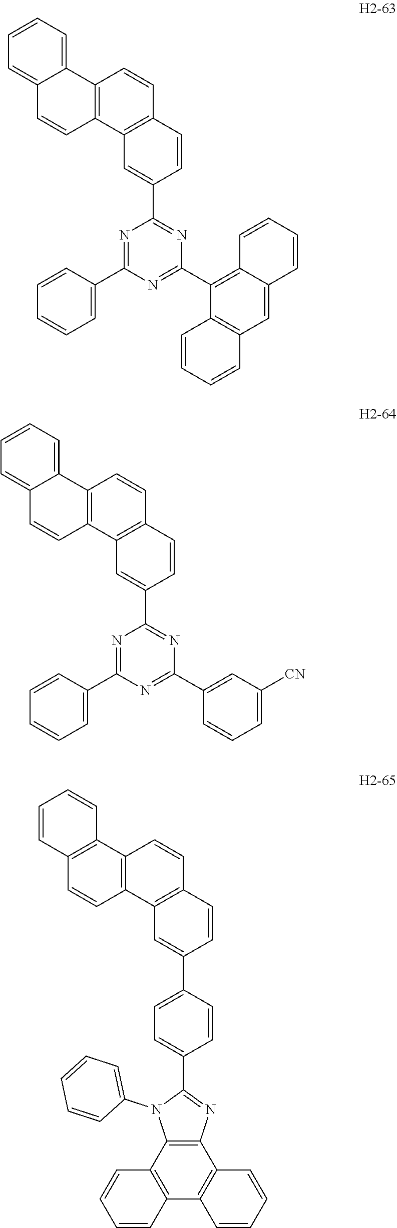 [SMILES: c1ccc(-c2nc(-c3ccc4ccc5c6ccccc6ccc5c4c3)nc(-c3c4ccccc4cc4ccccc34)n2)cc1 ; N#Cc1cccc(-c2nc(-c3ccccc3)nc(-c3ccc4ccc5c6ccccc6ccc5c4c3)n2)c1 ; c1ccc(-n2c(-c3ccc(-c4ccc5ccc6c7ccccc7ccc6c5c4)cc3)nc3c4ccccc4c4ccccc4c32)cc1]

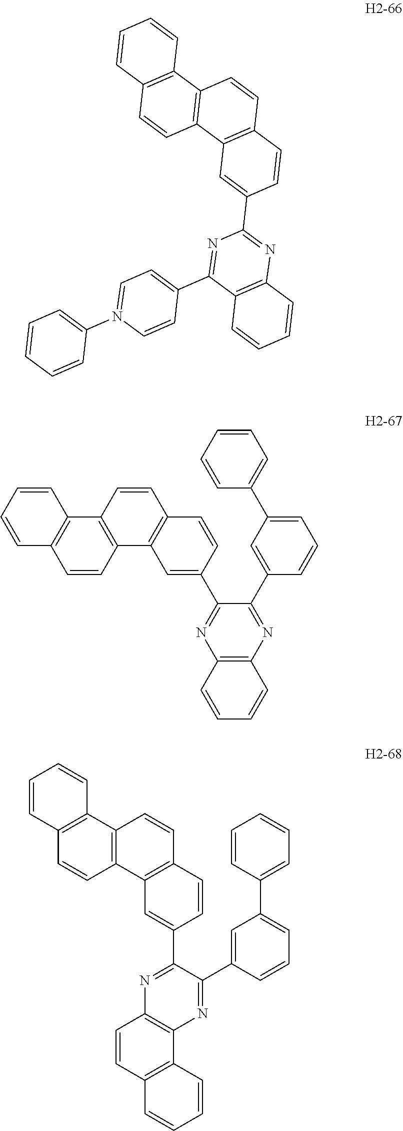

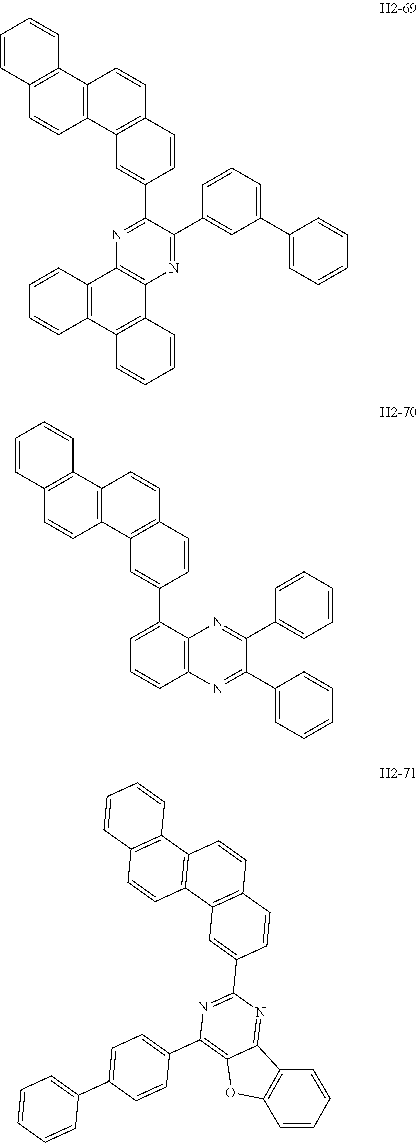 [SMILES: c1ccc(-c2cccc(-c3nc4c5ccccc5c5ccccc5c4nc3-c3ccc4ccc5c6ccccc6ccc5c4c3)c2)cc1 ; c1ccc(-c2nc3cccc(-c4ccc5ccc6c7ccccc7ccc6c5c4)c3nc2-c2ccccc2)cc1 ; c1ccc(-c2ccc(-c3nc(-c4ccc5ccc6c7ccccc7ccc6c5c4)nc4c3oc3ccccc34)cc2)cc1]

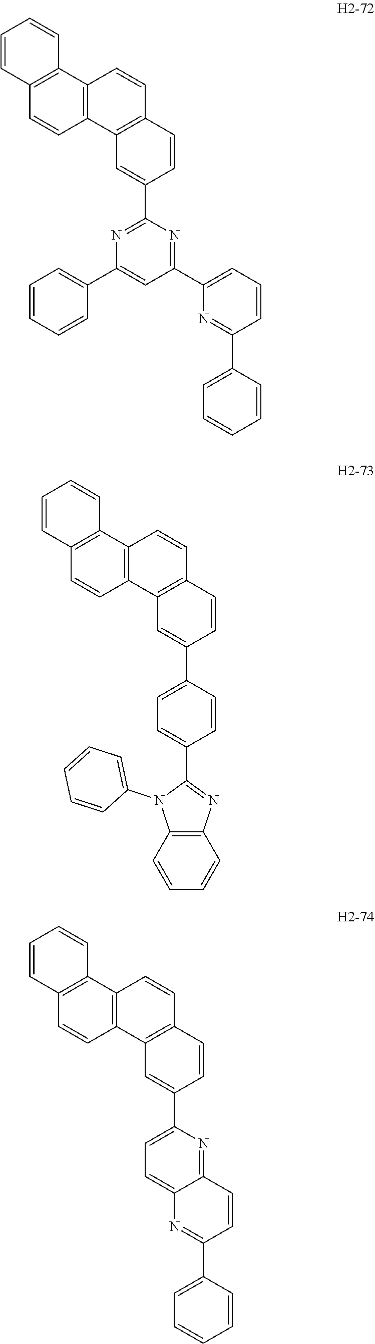 [SMILES: c1ccc(-c2cccc(-c3cc(-c4ccccc4)nc(-c4ccc5ccc6c7ccccc7ccc6c5c4)n3)n2)cc1 ; c1ccc(-n2c(-c3ccc(-c4ccc5ccc6c7ccccc7ccc6c5c4)cc3)nc3ccccc32)cc1 ; c1ccc(-c2ccc3nc(-c4ccc5ccc6c7ccccc7ccc6c5c4)ccc3n2)cc1]

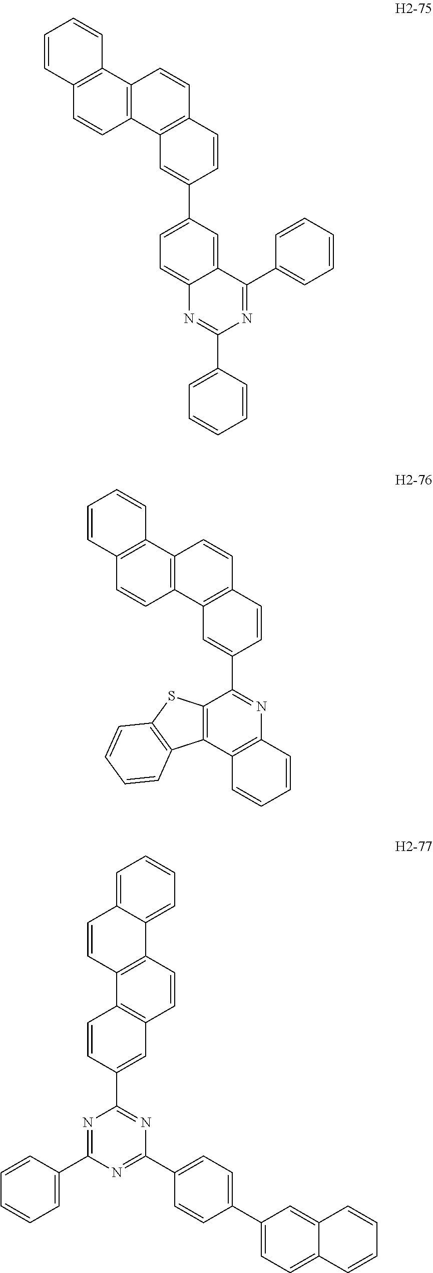 [SMILES: c1ccc(-c2nc(-c3ccccc3)c3cc(-c4ccc5ccc6c7ccccc7ccc6c5c4)ccc3n2)cc1 ; c1ccc2c(c1)ccc1c3cc(-c4nc5ccccc5c5c4sc4ccccc45)ccc3ccc21 ; c1ccc(-c2nc(-c3ccc(-c4ccc5ccccc5c4)cc3)nc(-c3ccc4c(ccc5c6ccccc6ccc45)c3)n2)cc1]

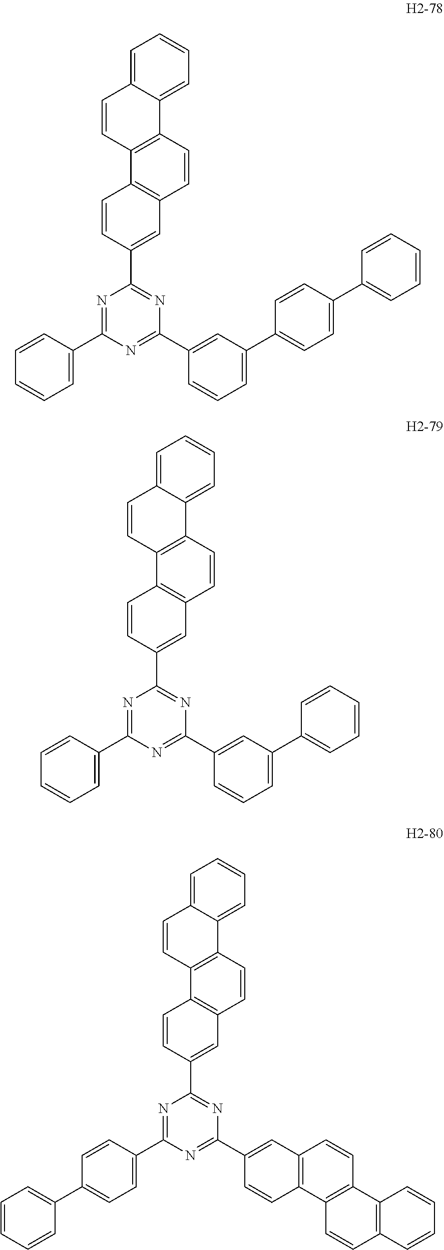 [SMILES: c1ccc(-c2ccc(-c3cccc(-c4nc(-c5ccccc5)nc(-c5ccc6c(ccc7c8ccccc8ccc67)c5)n4)c3)cc2)cc1 ; c1ccc(-c2cccc(-c3nc(-c4ccccc4)nc(-c4ccc5c(ccc6c7ccccc7ccc56)c4)n3)c2)cc1 ; c1ccc(-c2ccc(-c3nc(-c4ccc5c(ccc6c7ccccc7ccc56)c4)nc(-c4ccc5c(ccc6c7ccccc7ccc56)c4)n3)cc2)cc1]

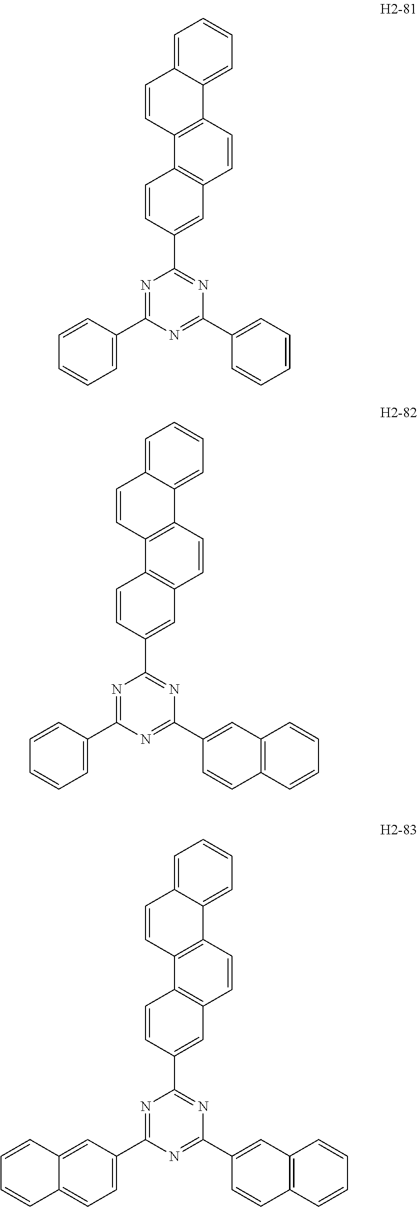 [SMILES: c1ccc(-c2nc(-c3ccccc3)nc(-c3ccc4c(ccc5c6ccccc6ccc45)c3)n2)cc1 ; c1ccc(-c2nc(-c3ccc4ccccc4c3)nc(-c3ccc4c(ccc5c6ccccc6ccc45)c3)n2)cc1 ; c1ccc2cc(-c3nc(-c4ccc5ccccc5c4)nc(-c4ccc5c(ccc6c7ccccc7ccc56)c4)n3)ccc2c1]

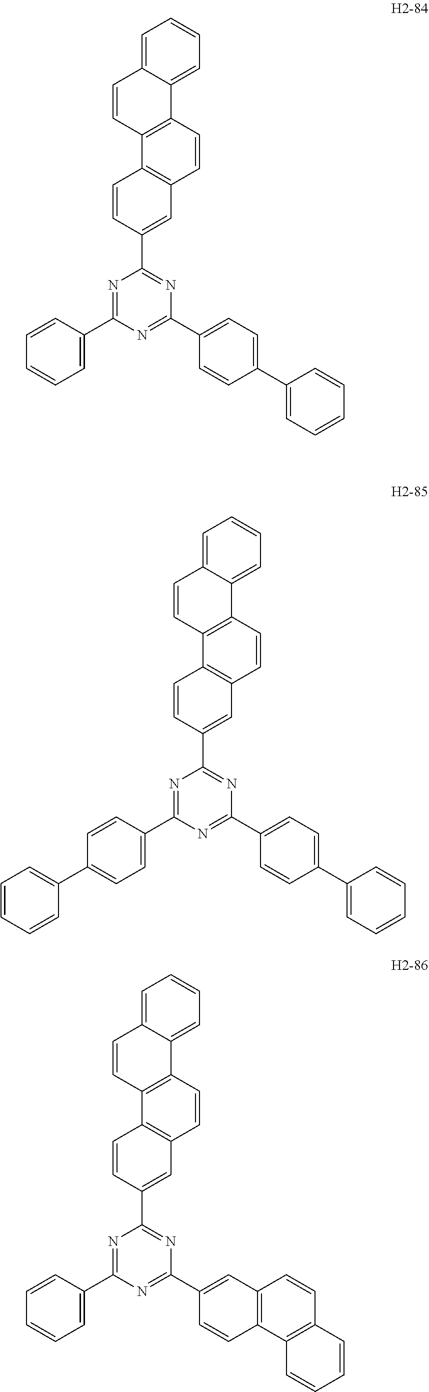 [SMILES: c1ccc(-c2ccc(-c3nc(-c4ccccc4)nc(-c4ccc5c(ccc6c7ccccc7ccc56)c4)n3)cc2)cc1 ; c1ccc(-c2ccc(-c3nc(-c4ccc(-c5ccccc5)cc4)nc(-c4ccc5c(ccc6c7ccccc7ccc56)c4)n3)cc2)cc1 ; c1ccc(-c2nc(-c3ccc4c(ccc5ccccc54)c3)nc(-c3ccc4c(ccc5c6ccccc6ccc45)c3)n2)cc1]

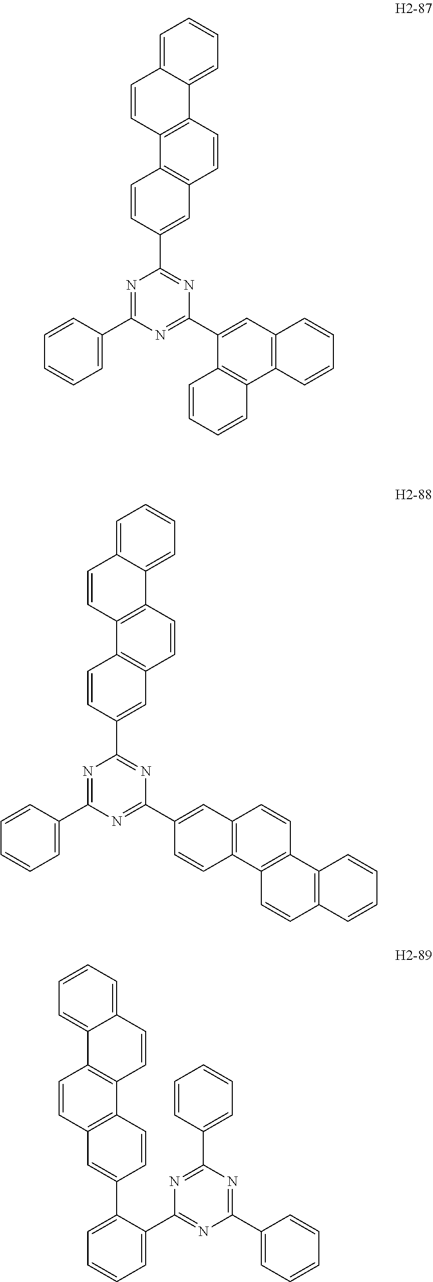 [SMILES: c1ccc(-c2nc(-c3ccc4c(ccc5c6ccccc6ccc45)c3)nc(-c3cc4ccccc4c4ccccc34)n2)cc1 ; c1ccc(-c2nc(-c3ccc4c(ccc5c6ccccc6ccc45)c3)nc(-c3ccc4c(ccc5c6ccccc6ccc45)c3)n2)cc1 ; c1ccc(-c2nc(-c3ccccc3)nc(-c3ccccc3-c3ccc4c(ccc5c6ccccc6ccc45)c3)n2)cc1]

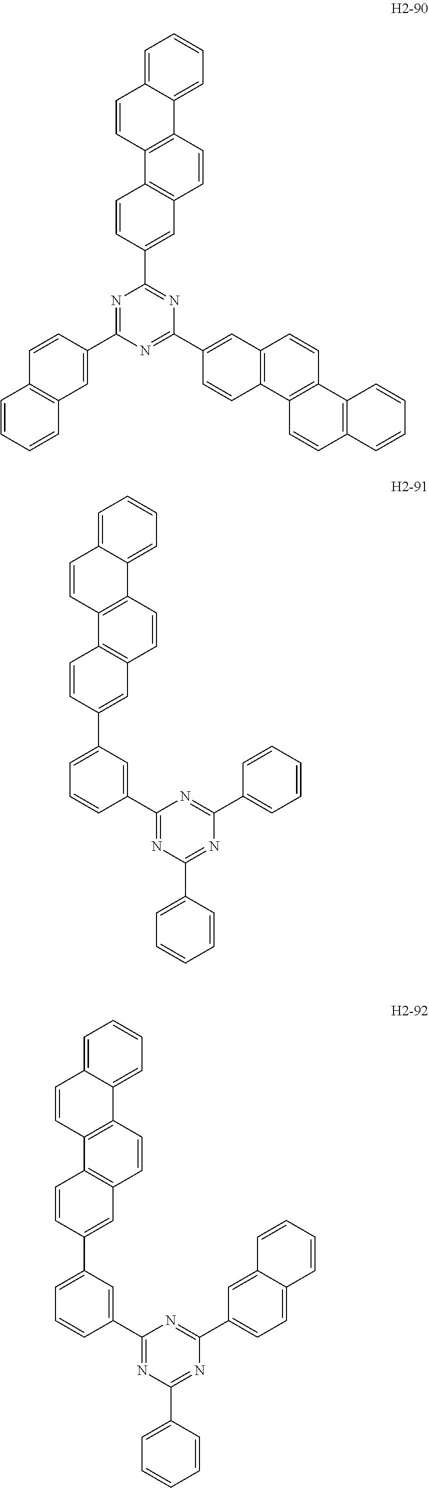 [SMILES: c1ccc2cc(-c3nc(-c4ccc5c(ccc6c7ccccc7ccc56)c4)nc(-c4ccc5c(ccc6c7ccccc7ccc56)c4)n3)ccc2c1 ; c1ccc(-c2nc(-c3ccccc3)nc(-c3cccc(-c4ccc5c(ccc6c7ccccc7ccc56)c4)c3)n2)cc1 ; c1ccc(-c2nc(-c3cccc(-c4ccc5c(ccc6c7ccccc7ccc56)c4)c3)nc(-c3ccc4ccccc4c3)n2)cc1]

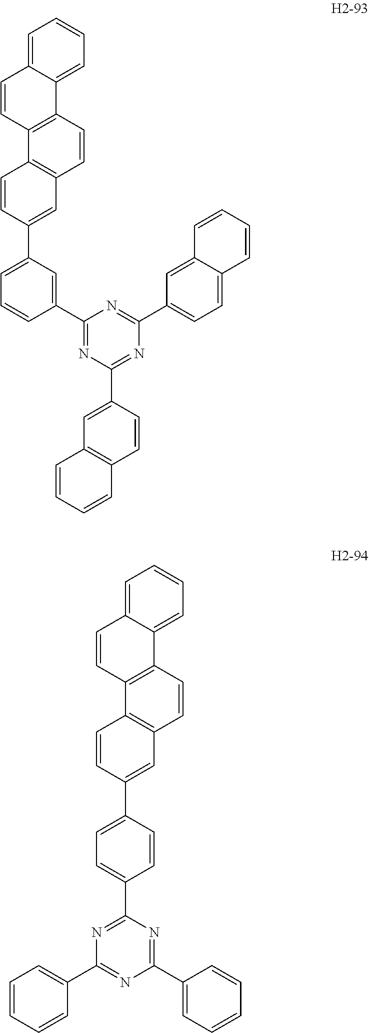 [SMILES: c1cc(-c2ccc3c(ccc4c5ccccc5ccc34)c2)cc(-c2nc(-c3ccc4ccccc4c3)nc(-c3ccc4ccccc4c3)n2)c1 ; c1ccc(-c2nc(-c3ccccc3)nc(-c3ccc(-c4ccc5c(ccc6c7ccccc7ccc56)c4)cc3)n2)cc1]

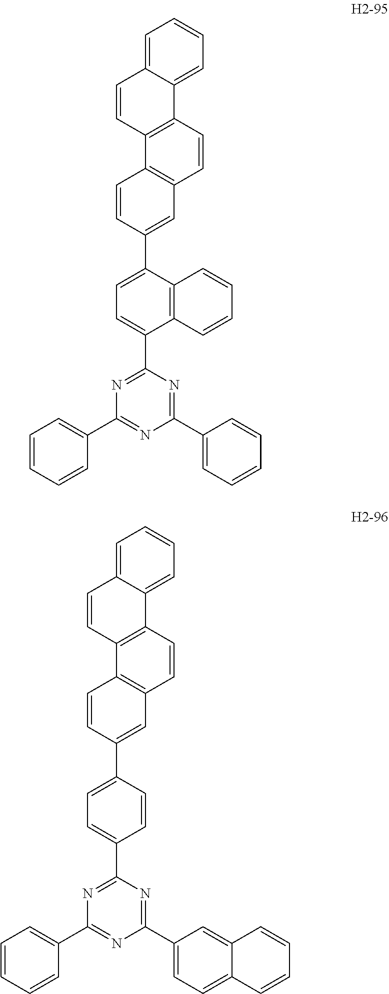 [SMILES: c1ccc(-c2nc(-c3ccccc3)nc(-c3ccc(-c4ccc5c(ccc6c7ccccc7ccc56)c4)c4ccccc34)n2)cc1 ; c1ccc(-c2nc(-c3ccc(-c4ccc5c(ccc6c7ccccc7ccc56)c4)cc3)nc(-c3ccc4ccccc4c3)n2)cc1]

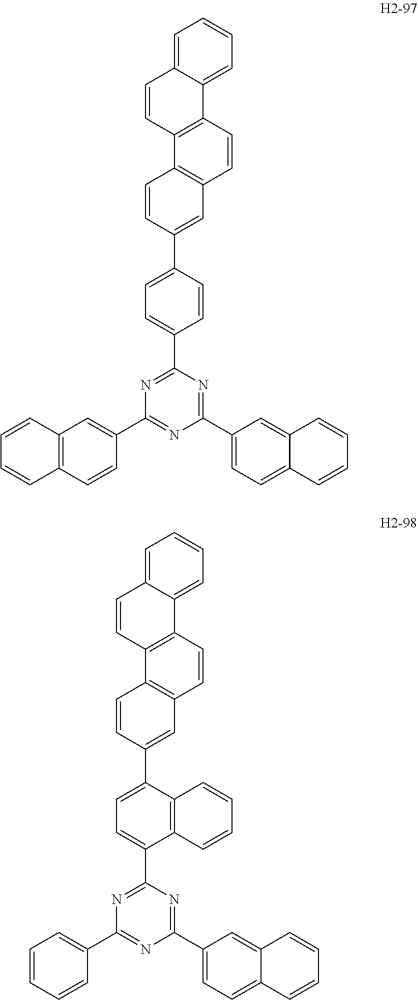 [SMILES: c1ccc2cc(-c3nc(-c4ccc(-c5ccc6c(ccc7c8ccccc8ccc67)c5)cc4)nc(-c4ccc5ccccc5c4)n3)ccc2c1 ; c1ccc(-c2nc(-c3ccc4ccccc4c3)nc(-c3ccc(-c4ccc5c(ccc6c7ccccc7ccc56)c4)c4ccccc34)n2)cc1]

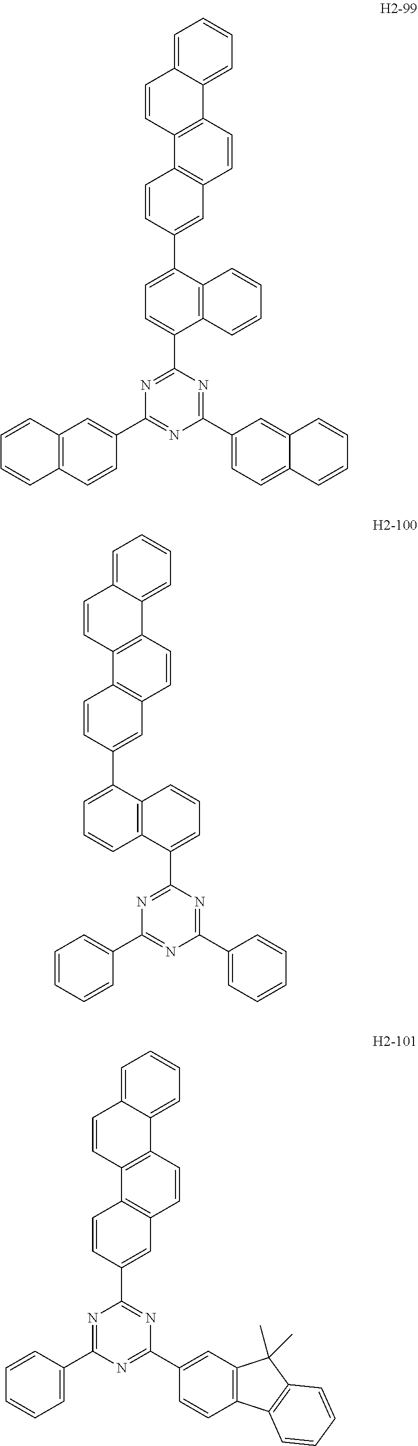 [SMILES: c1ccc2cc(-c3nc(-c4ccc5ccccc5c4)nc(-c4ccc(-c5ccc6c(ccc7c8ccccc8ccc67)c5)c5ccccc45)n3)ccc2c1 ; c1ccc(-c2nc(-c3ccccc3)nc(-c3cccc4c(-c5ccc6c(ccc7c8ccccc8ccc67)c5)cccc34)n2)cc1 ; CC1(C)c2ccccc2-c2ccc(-c3nc(-c4ccccc4)nc(-c4ccc5c(ccc6c7ccccc7ccc56)c4)n3)cc21]

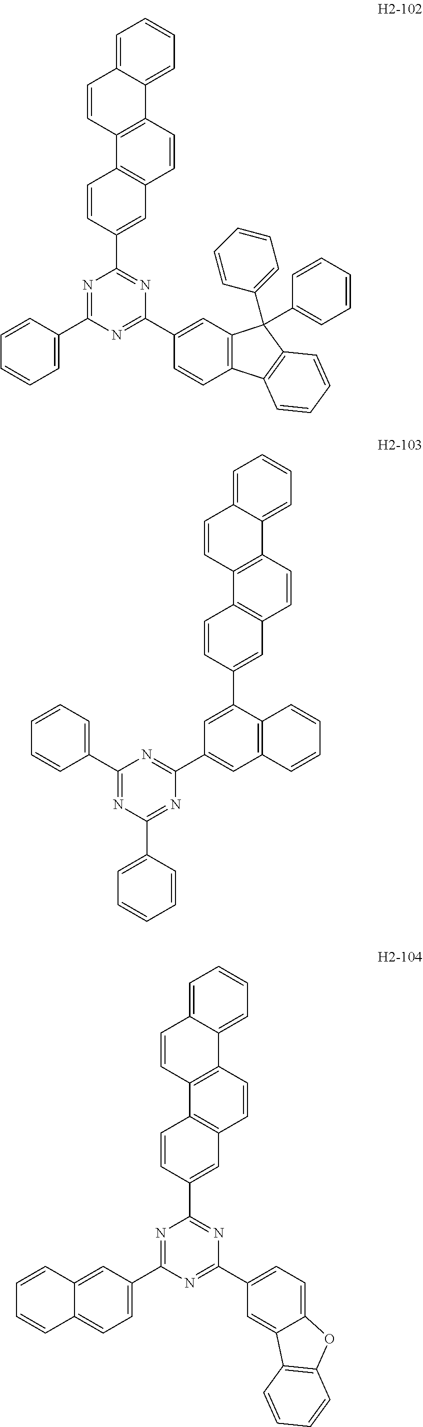 [SMILES: c1ccc(-c2nc(-c3ccc4c(c3)C(c3ccccc3)(c3ccccc3)c3ccccc3-4)nc(-c3ccc4c(ccc5c6ccccc6ccc45)c3)n2)cc1 ; c1ccc(-c2nc(-c3ccccc3)nc(-c3cc(-c4ccc5c(ccc6c7ccccc7ccc56)c4)c4ccccc4c3)n2)cc1 ; c1ccc2cc(-c3nc(-c4ccc5c(ccc6c7ccccc7ccc56)c4)nc(-c4ccc5oc6ccccc6c5c4)n3)ccc2c1]

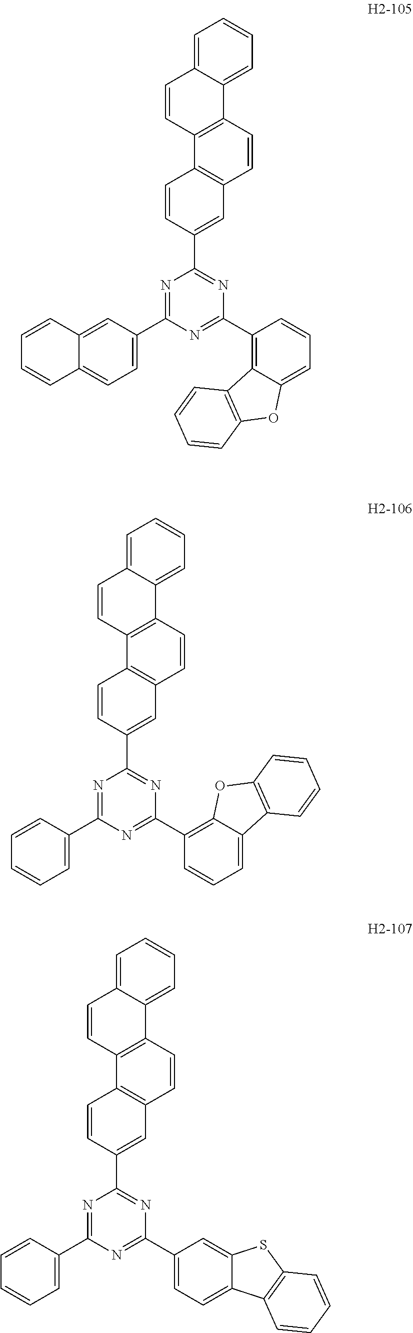 [SMILES: c1ccc2cc(-c3nc(-c4ccc5c(ccc6c7ccccc7ccc56)c4)nc(-c4cccc5oc6ccccc6c45)n3)ccc2c1 ; c1ccc(-c2nc(-c3ccc4c(ccc5c6ccccc6ccc45)c3)nc(-c3cccc4c3oc3ccccc34)n2)cc1 ; c1ccc(-c2nc(-c3ccc4c(ccc5c6ccccc6ccc45)c3)nc(-c3ccc4c(c3)sc3ccccc34)n2)cc1]

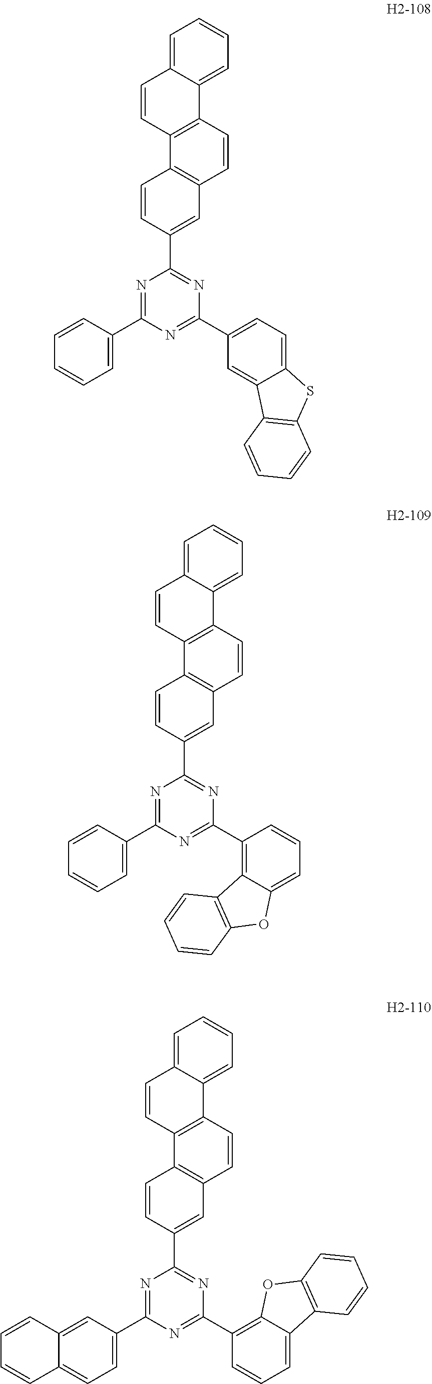 [SMILES: c1ccc(-c2nc(-c3ccc4c(ccc5c6ccccc6ccc45)c3)nc(-c3ccc4sc5ccccc5c4c3)n2)cc1 ; c1ccc(-c2nc(-c3ccc4c(ccc5c6ccccc6ccc45)c3)nc(-c3cccc4oc5ccccc5c34)n2)cc1 ; c1ccc2cc(-c3nc(-c4ccc5c(ccc6c7ccccc7ccc56)c4)nc(-c4cccc5c4oc4ccccc45)n3)ccc2c1]

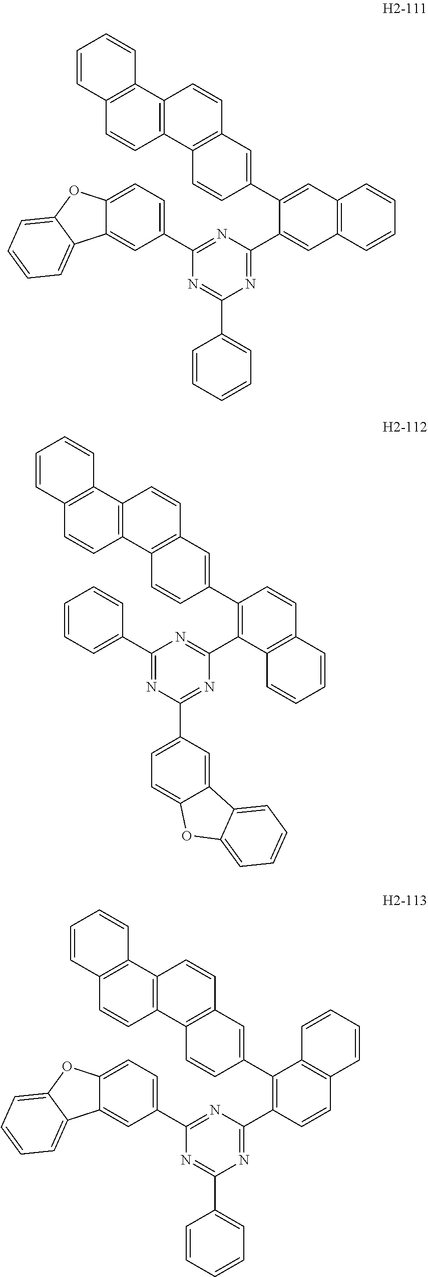 [SMILES: c1ccc(-c2nc(-c3ccc4oc5ccccc5c4c3)nc(-c3cc4ccccc4cc3-c3ccc4c(ccc5c6ccccc6ccc45)c3)n2)cc1 ; c1ccc(-c2nc(-c3ccc4oc5ccccc5c4c3)nc(-c3c(-c4ccc5c(ccc6c7ccccc7ccc56)c4)ccc4ccccc34)n2)cc1 ; c1ccc(-c2nc(-c3ccc4oc5ccccc5c4c3)nc(-c3ccc4ccccc4c3-c3ccc4c(ccc5c6ccccc6ccc45)c3)n2)cc1]

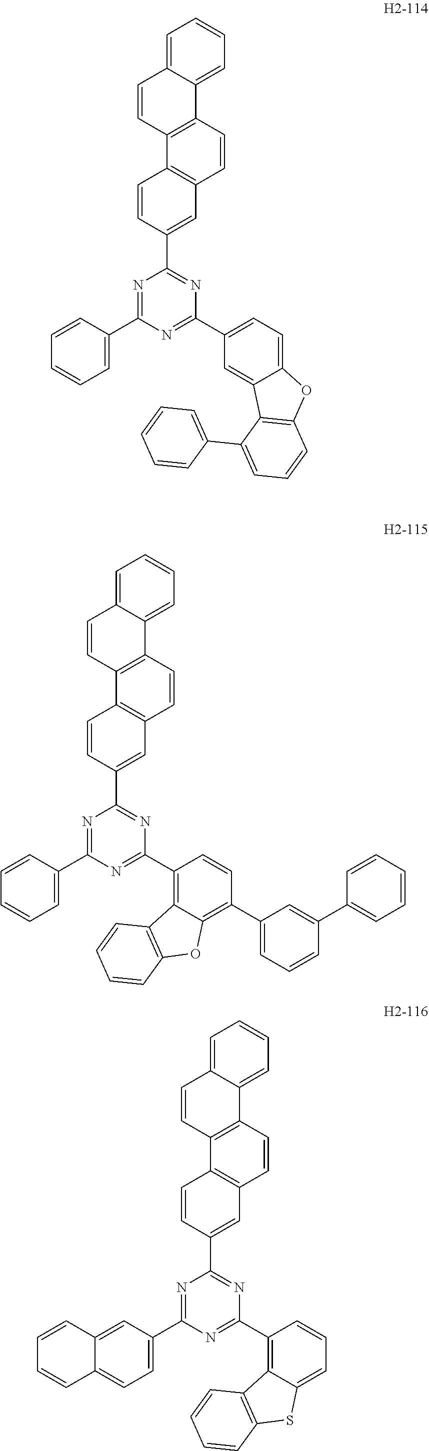 [SMILES: c1ccc(-c2nc(-c3ccc4c(ccc5c6ccccc6ccc45)c3)nc(-c3ccc4oc5cccc(-c6ccccc6)c5c4c3)n2)cc1 ; c1ccc(-c2cccc(-c3ccc(-c4nc(-c5ccccc5)nc(-c5ccc6c(ccc7c8ccccc8ccc67)c5)n4)c4c3oc3ccccc34)c2)cc1 ; c1ccc2cc(-c3nc(-c4ccc5c(ccc6c7ccccc7ccc56)c4)nc(-c4cccc5sc6ccccc6c45)n3)ccc2c1]

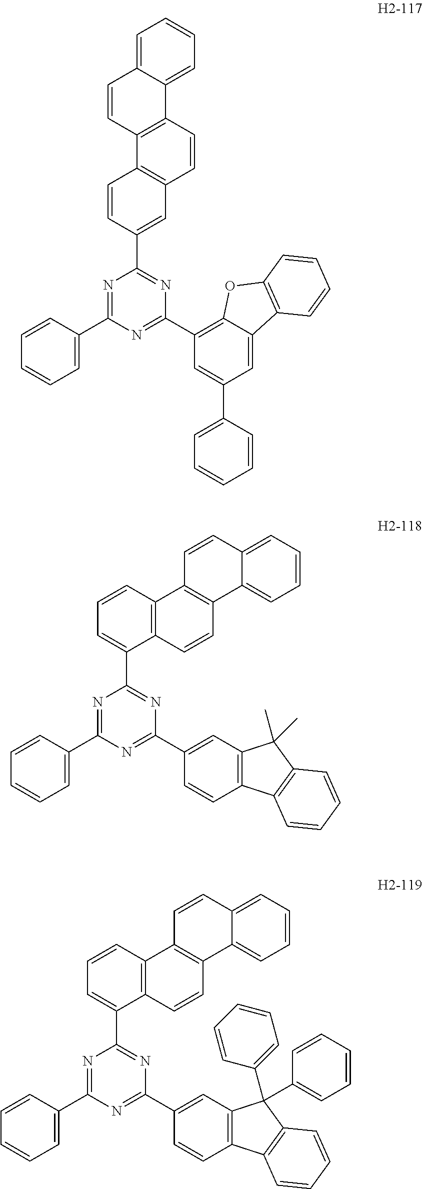 [SMILES: c1ccc(-c2cc(-c3nc(-c4ccccc4)nc(-c4ccc5c(ccc6c7ccccc7ccc56)c4)n3)c3oc4ccccc4c3c2)cc1 ; CC1(C)c2ccccc2-c2ccc(-c3nc(-c4ccccc4)nc(-c4cccc5c4ccc4c6ccccc6ccc54)n3)cc21 ; c1ccc(-c2nc(-c3ccc4c(c3)C(c3ccccc3)(c3ccccc3)c3ccccc3-4)nc(-c3cccc4c3ccc3c5ccccc5ccc43)n2)cc1]

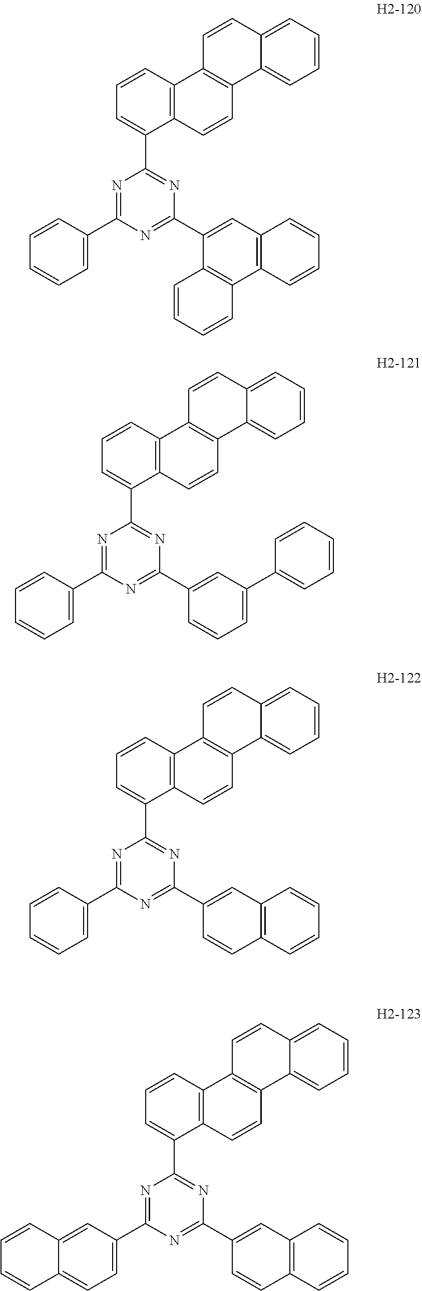

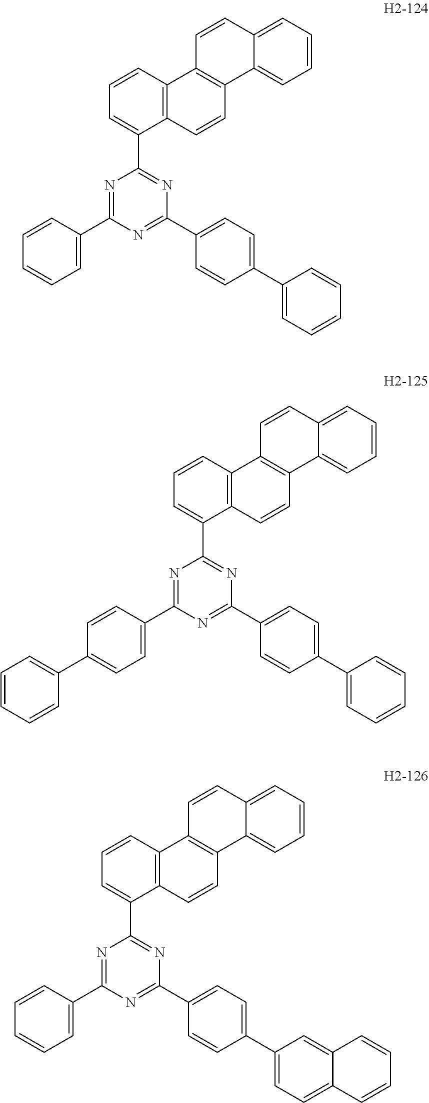 [SMILES: c1ccc(-c2ccc(-c3nc(-c4ccccc4)nc(-c4cccc5c4ccc4c6ccccc6ccc54)n3)cc2)cc1 ; c1ccc(-c2ccc(-c3nc(-c4ccc(-c5ccccc5)cc4)nc(-c4cccc5c4ccc4c6ccccc6ccc54)n3)cc2)cc1 ; c1ccc(-c2nc(-c3ccc(-c4ccc5ccccc5c4)cc3)nc(-c3cccc4c3ccc3c5ccccc5ccc43)n2)cc1]

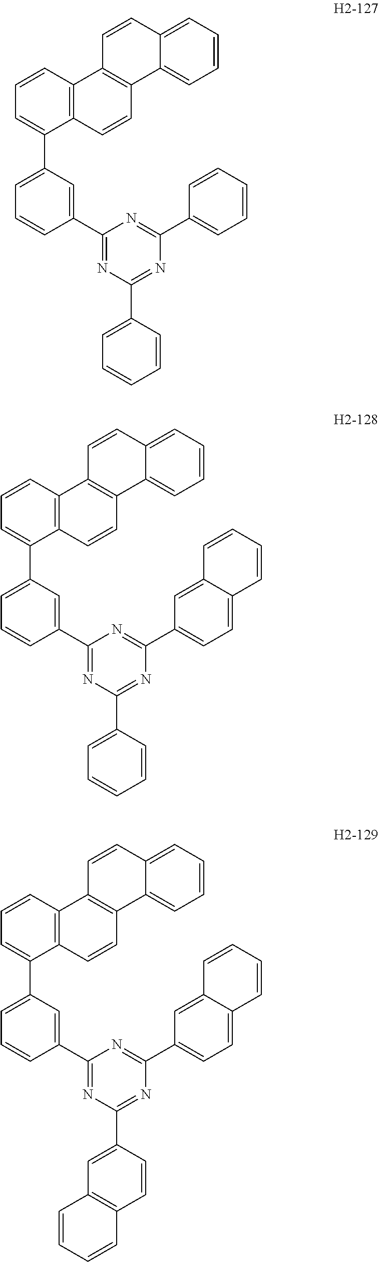

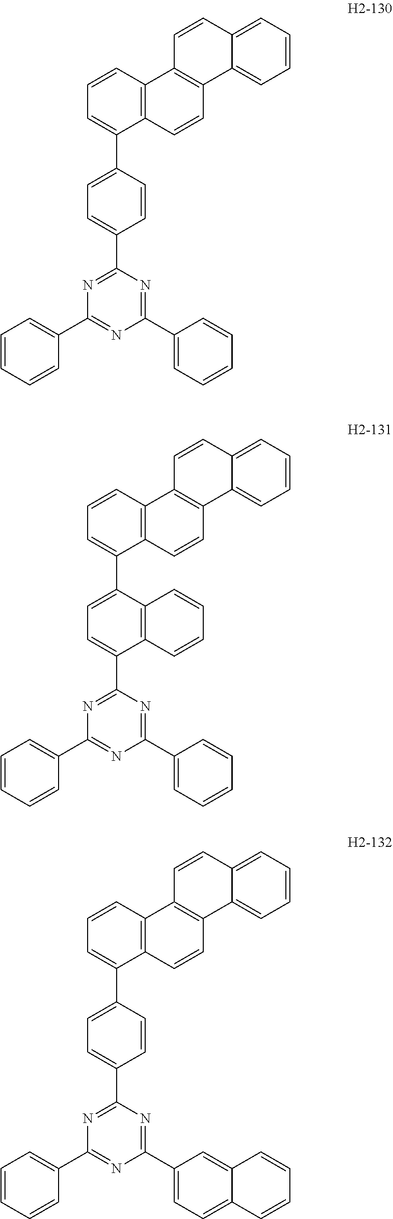

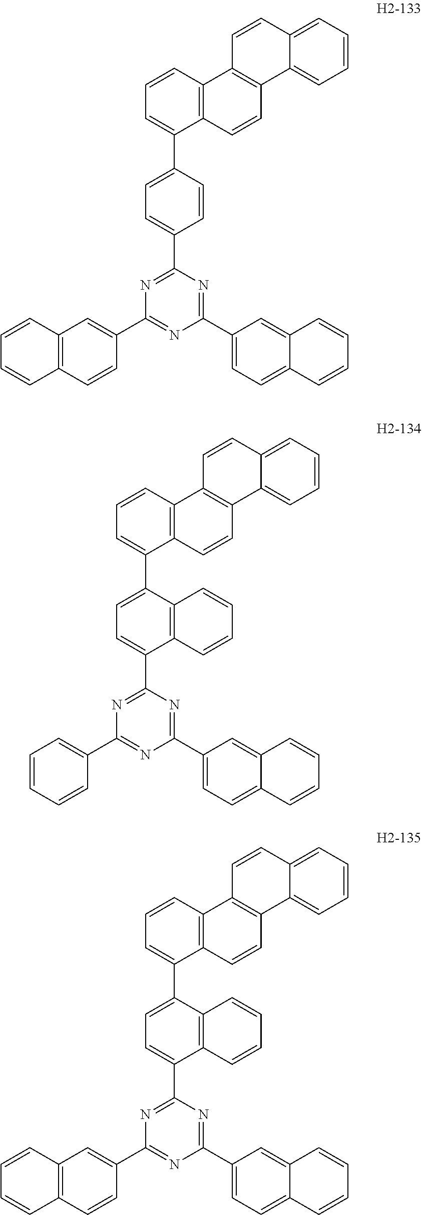 [SMILES: c1ccc2cc(-c3nc(-c4ccc(-c5cccc6c5ccc5c7ccccc7ccc65)cc4)nc(-c4ccc5ccccc5c4)n3)ccc2c1 ; c1ccc(-c2nc(-c3ccc4ccccc4c3)nc(-c3ccc(-c4cccc5c4ccc4c6ccccc6ccc54)c4ccccc34)n2)cc1 ; c1ccc2cc(-c3nc(-c4ccc5ccccc5c4)nc(-c4ccc(-c5cccc6c5ccc5c7ccccc7ccc65)c5ccccc45)n3)ccc2c1]

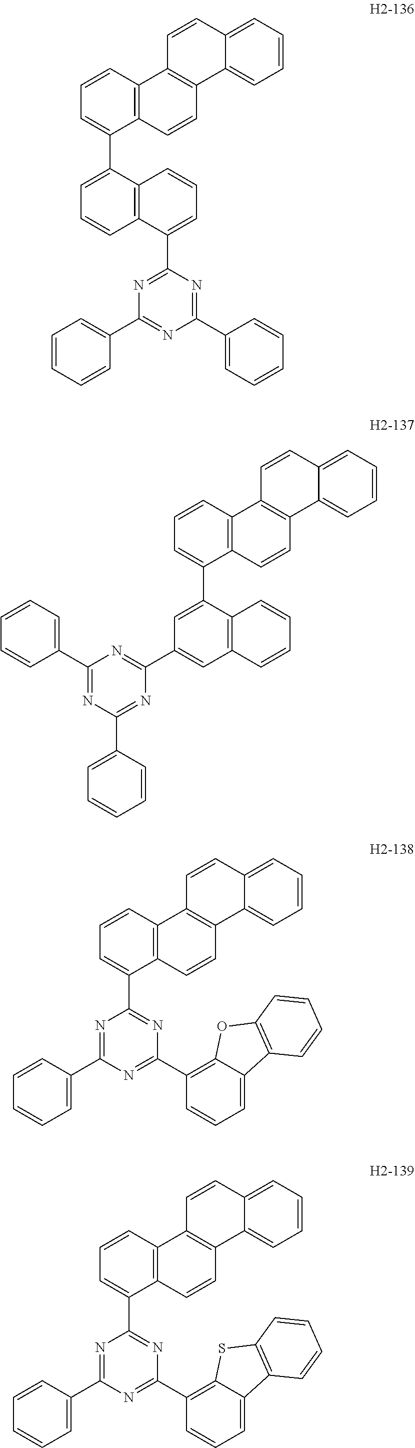 [SMILES: c1ccc(-c2nc(-c3ccccc3)nc(-c3cccc4c(-c5cccc6c5ccc5c7ccccc7ccc65)cccc34)n2)cc1 ; c1ccc(-c2nc(-c3ccccc3)nc(-c3cc(-c4cccc5c4ccc4c6ccccc6ccc54)c4ccccc4c3)n2)cc1 ; c1ccc(-c2nc(-c3cccc4c3ccc3c5ccccc5ccc43)nc(-c3cccc4c3oc3ccccc34)n2)cc1 ; c1ccc(-c2nc(-c3cccc4c3ccc3c5ccccc5ccc43)nc(-c3cccc4c3sc3ccccc34)n2)cc1]

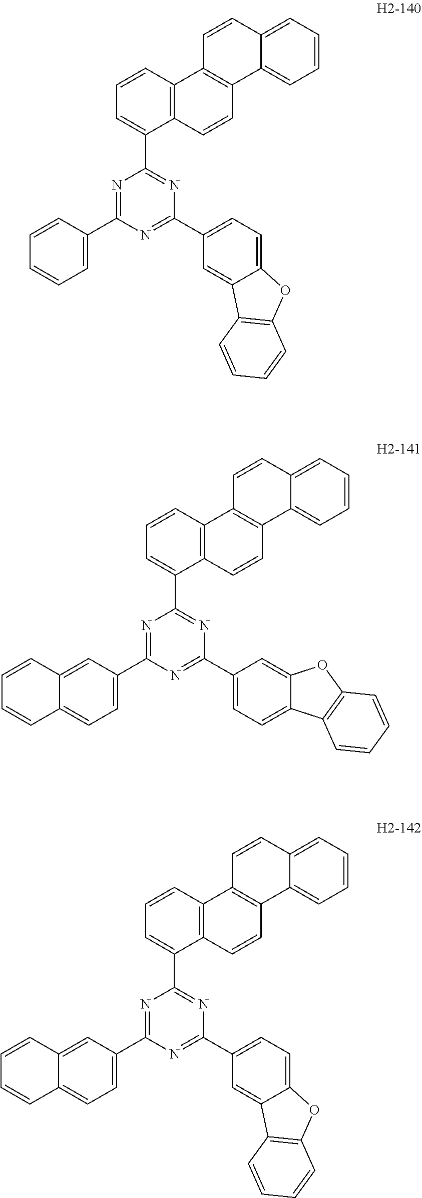 [SMILES: c1ccc(-c2nc(-c3ccc4oc5ccccc5c4c3)nc(-c3cccc4c3ccc3c5ccccc5ccc43)n2)cc1 ; c1ccc2cc(-c3nc(-c4ccc5c(c4)oc4ccccc45)nc(-c4cccc5c4ccc4c6ccccc6ccc54)n3)ccc2c1 ; c1ccc2cc(-c3nc(-c4ccc5oc6ccccc6c5c4)nc(-c4cccc5c4ccc4c6ccccc6ccc54)n3)ccc2c1]

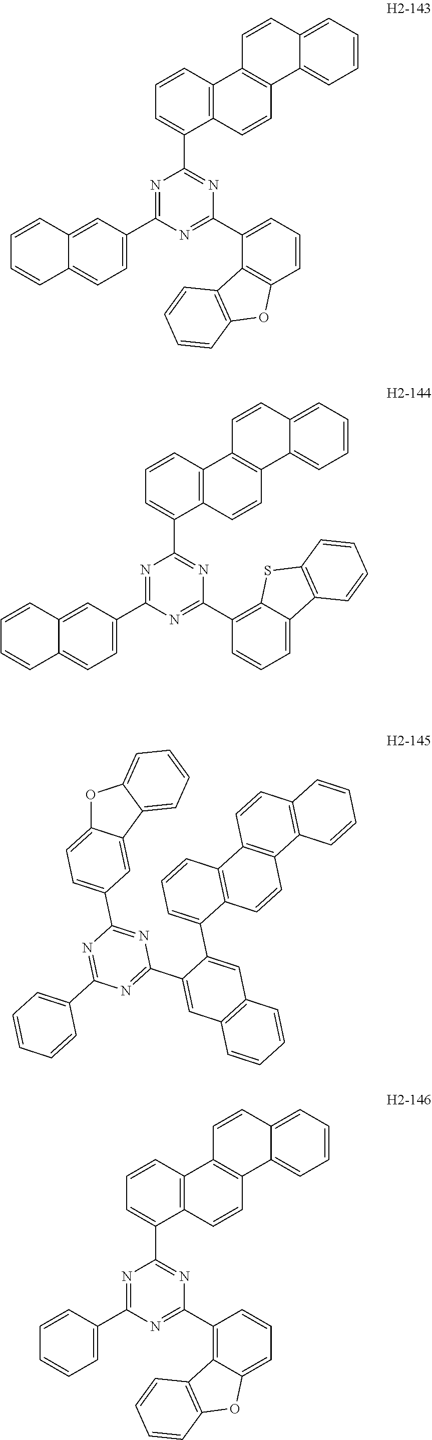 [SMILES: c1ccc2cc(-c3nc(-c4cccc5c4ccc4c6ccccc6ccc54)nc(-c4cccc5oc6ccccc6c45)n3)ccc2c1 ; c1ccc2cc(-c3nc(-c4cccc5c4ccc4c6ccccc6ccc54)nc(-c4cccc5c4sc4ccccc45)n3)ccc2c1 ; c1ccc(-c2nc(-c3ccc4oc5ccccc5c4c3)nc(-c3cc4ccccc4cc3-c3cccc4c3ccc3c5ccccc5ccc43)n2)cc1 ; c1ccc(-c2nc(-c3cccc4c3ccc3c5ccccc5ccc43)nc(-c3cccc4oc5ccccc5c34)n2)cc1]

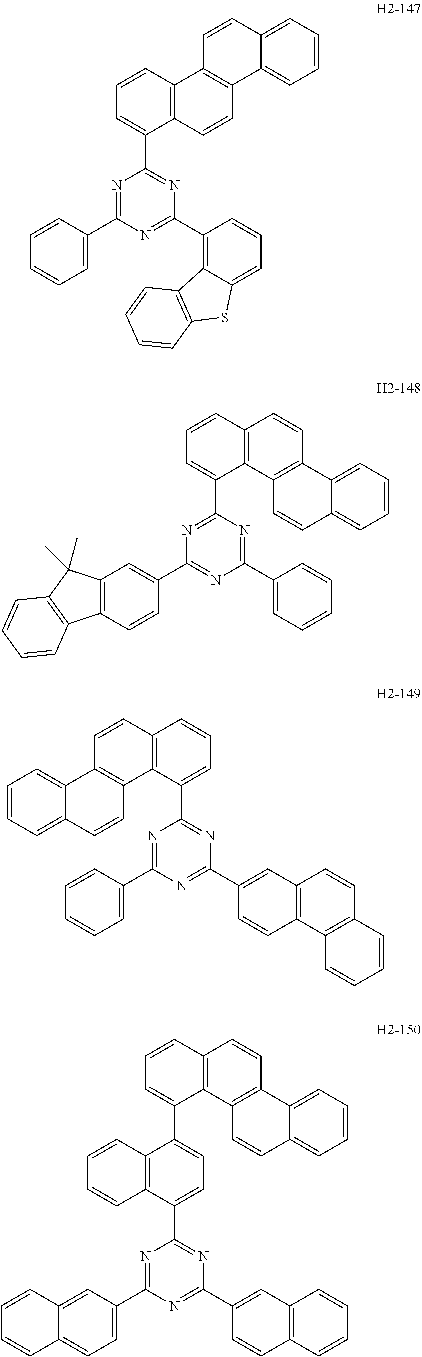 [SMILES: c1ccc(-c2nc(-c3cccc4c3ccc3c5ccccc5ccc43)nc(-c3cccc4sc5ccccc5c34)n2)cc1 ; CC1(C)c2ccccc2-c2ccc(-c3nc(-c4ccccc4)nc(-c4cccc5ccc6c7ccccc7ccc6c45)n3)cc21 ; c1ccc(-c2nc(-c3ccc4c(ccc5ccccc54)c3)nc(-c3cccc4ccc5c6ccccc6ccc5c34)n2)cc1 ; c1ccc2cc(-c3nc(-c4ccc5ccccc5c4)nc(-c4ccc(-c5cccc6ccc7c8ccccc8ccc7c56)c5ccccc45)n3)ccc2c1]

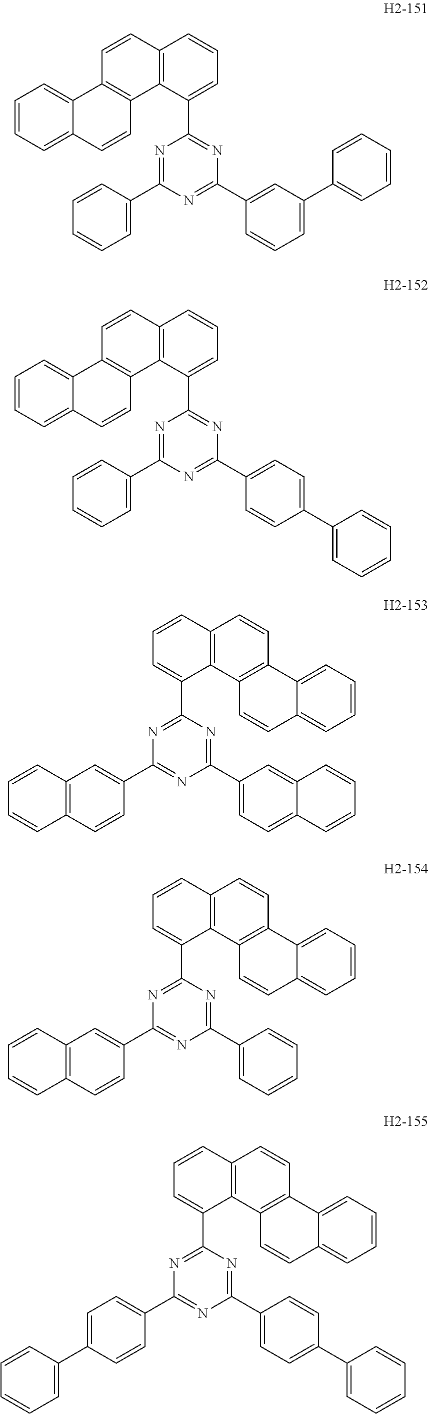 [SMILES: c1ccc(-c2cccc(-c3nc(-c4ccccc4)nc(-c4cccc5ccc6c7ccccc7ccc6c45)n3)c2)cc1 ; c1ccc(-c2ccc(-c3nc(-c4ccccc4)nc(-c4cccc5ccc6c7ccccc7ccc6c45)n3)cc2)cc1 ; c1ccc2cc(-c3nc(-c4ccc5ccccc5c4)nc(-c4cccc5ccc6c7ccccc7ccc6c45)n3)ccc2c1 ; c1ccc(-c2nc(-c3ccc4ccccc4c3)nc(-c3cccc4ccc5c6ccccc6ccc5c34)n2)cc1 ; c1ccc(-c2ccc(-c3nc(-c4ccc(-c5ccccc5)cc4)nc(-c4cccc5ccc6c7ccccc7ccc6c45)n3)cc2)cc1]

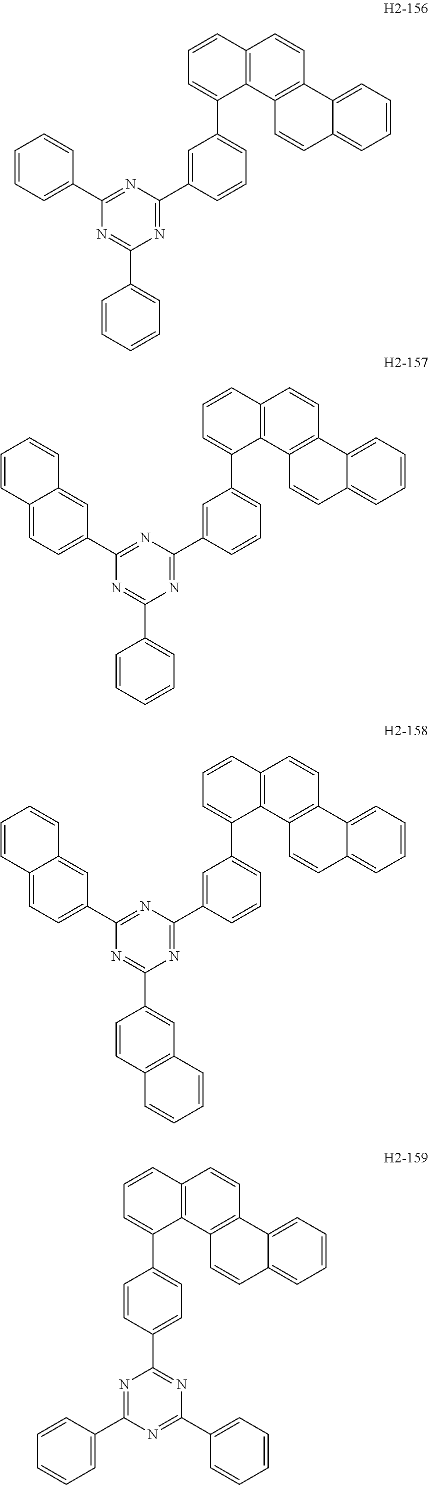 [SMILES: c1ccc(-c2nc(-c3ccccc3)nc(-c3cccc(-c4cccc5ccc6c7ccccc7ccc6c45)c3)n2)cc1 ; c1ccc(-c2nc(-c3cccc(-c4cccc5ccc6c7ccccc7ccc6c45)c3)nc(-c3ccc4ccccc4c3)n2)cc1 ; c1cc(-c2nc(-c3ccc4ccccc4c3)nc(-c3ccc4ccccc4c3)n2)cc(-c2cccc3ccc4c5ccccc5ccc4c23)c1 ; c1ccc(-c2nc(-c3ccccc3)nc(-c3ccc(-c4cccc5ccc6c7ccccc7ccc6c45)cc3)n2)cc1]

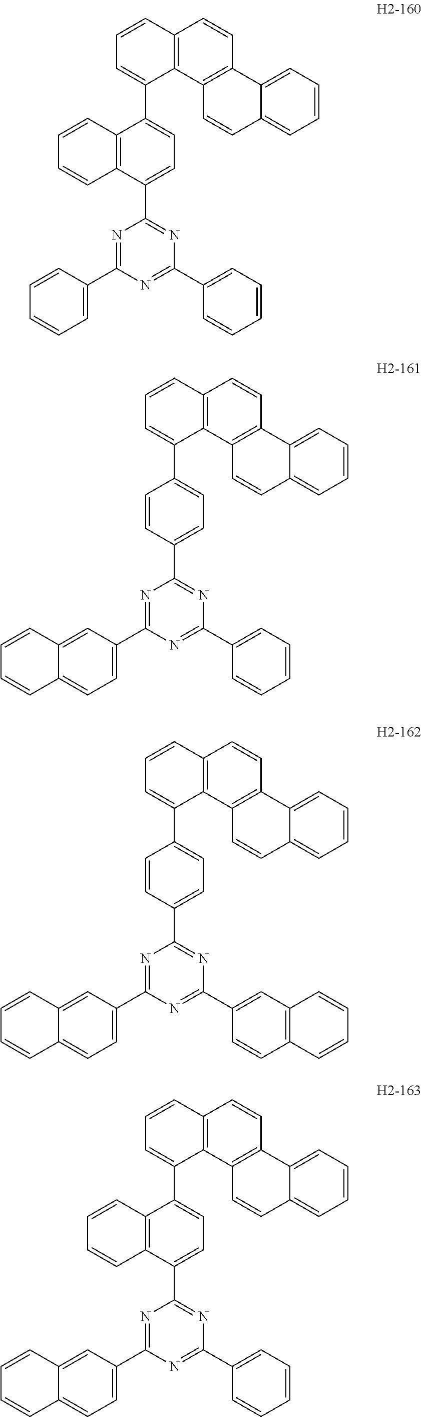

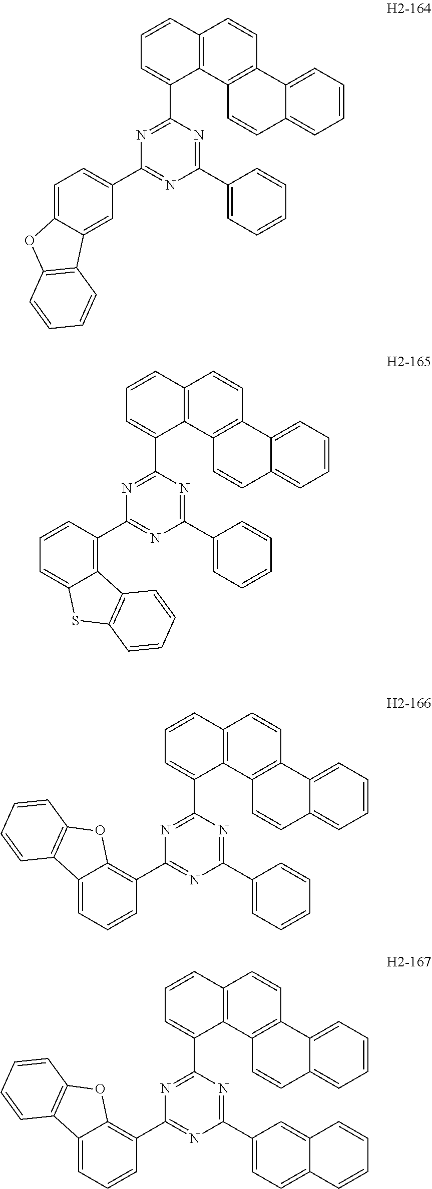 [SMILES: c1ccc(-c2nc(-c3ccc4oc5ccccc5c4c3)nc(-c3cccc4ccc5c6ccccc6ccc5c34)n2)cc1 ; c1ccc(-c2nc(-c3cccc4ccc5c6ccccc6ccc5c34)nc(-c3cccc4sc5ccccc5c34)n2)cc1 ; c1ccc(-c2nc(-c3cccc4c3oc3ccccc34)nc(-c3cccc4ccc5c6ccccc6ccc5c34)n2)cc1 ; c1ccc2cc(-c3nc(-c4cccc5c4oc4ccccc45)nc(-c4cccc5ccc6c7ccccc7ccc6c45)n3)ccc2c1]

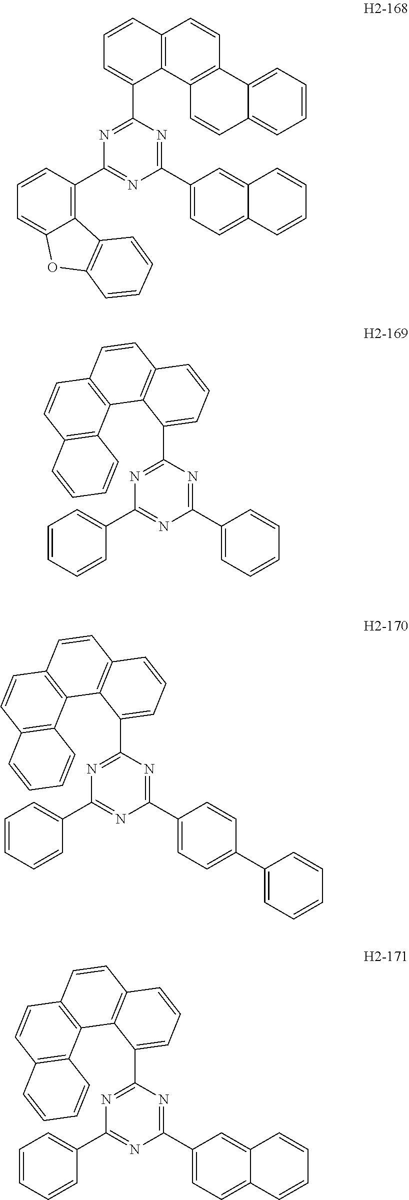 [SMILES: c1ccc2cc(-c3nc(-c4cccc5ccc6c7ccccc7ccc6c45)nc(-c4cccc5oc6ccccc6c45)n3)ccc2c1 ; c1ccc(-c2nc(-c3ccccc3)nc(-c3cccc4ccc5ccc6ccccc6c5c34)n2)cc1 ; c1ccc(-c2ccc(-c3nc(-c4ccccc4)nc(-c4cccc5ccc6ccc7ccccc7c6c45)n3)cc2)cc1 ; c1ccc(-c2nc(-c3ccc4ccccc4c3)nc(-c3cccc4ccc5ccc6ccccc6c5c34)n2)cc1]

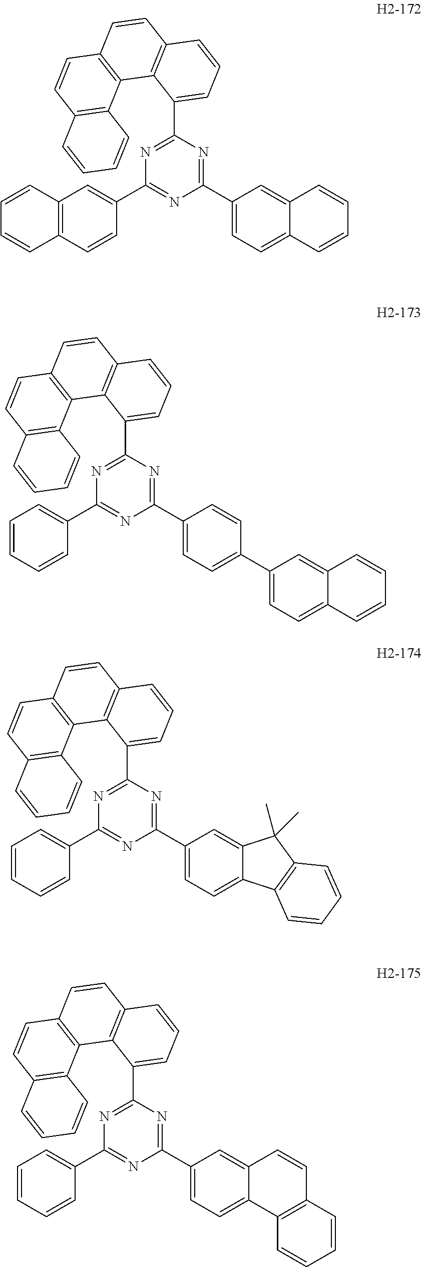 [SMILES: c1ccc2cc(-c3nc(-c4ccc5ccccc5c4)nc(-c4cccc5ccc6ccc7ccccc7c6c45)n3)ccc2c1 ; c1ccc(-c2nc(-c3ccc(-c4ccc5ccccc5c4)cc3)nc(-c3cccc4ccc5ccc6ccccc6c5c34)n2)cc1 ; CC1(C)c2ccccc2-c2ccc(-c3nc(-c4ccccc4)nc(-c4cccc5ccc6ccc7ccccc7c6c45)n3)cc21 ; c1ccc(-c2nc(-c3ccc4c(ccc5ccccc54)c3)nc(-c3cccc4ccc5ccc6ccccc6c5c34)n2)cc1]

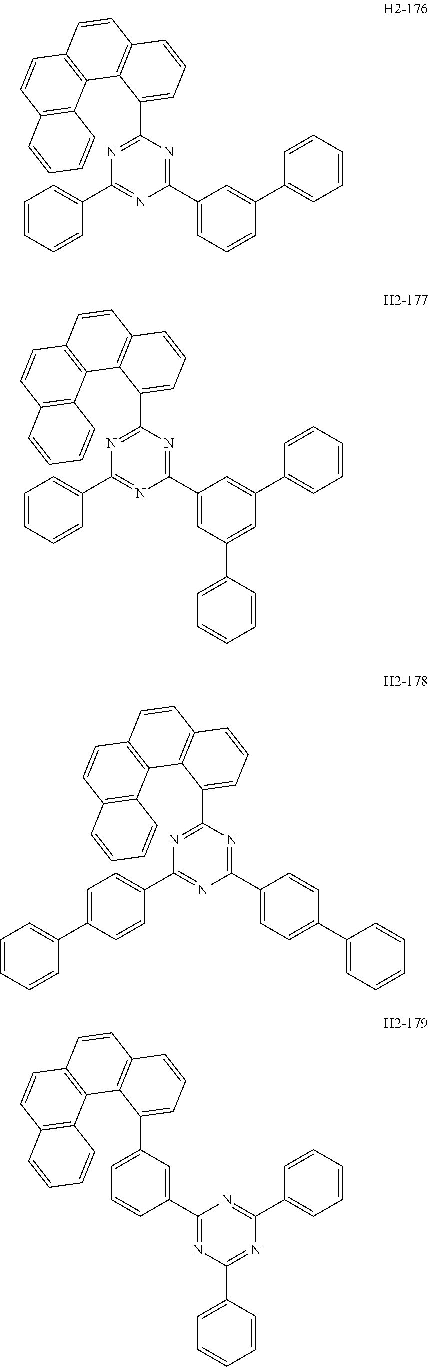 [SMILES: c1ccc(-c2cccc(-c3nc(-c4ccccc4)nc(-c4cccc5ccc6ccc7ccccc7c6c45)n3)c2)cc1 ; c1ccc(-c2cc(-c3ccccc3)cc(-c3nc(-c4ccccc4)nc(-c4cccc5ccc6ccc7ccccc7c6c45)n3)c2)cc1 ; c1ccc(-c2ccc(-c3nc(-c4ccc(-c5ccccc5)cc4)nc(-c4cccc5ccc6ccc7ccccc7c6c45)n3)cc2)cc1 ; c1ccc(-c2nc(-c3ccccc3)nc(-c3cccc(-c4cccc5ccc6ccc7ccccc7c6c45)c3)n2)cc1]

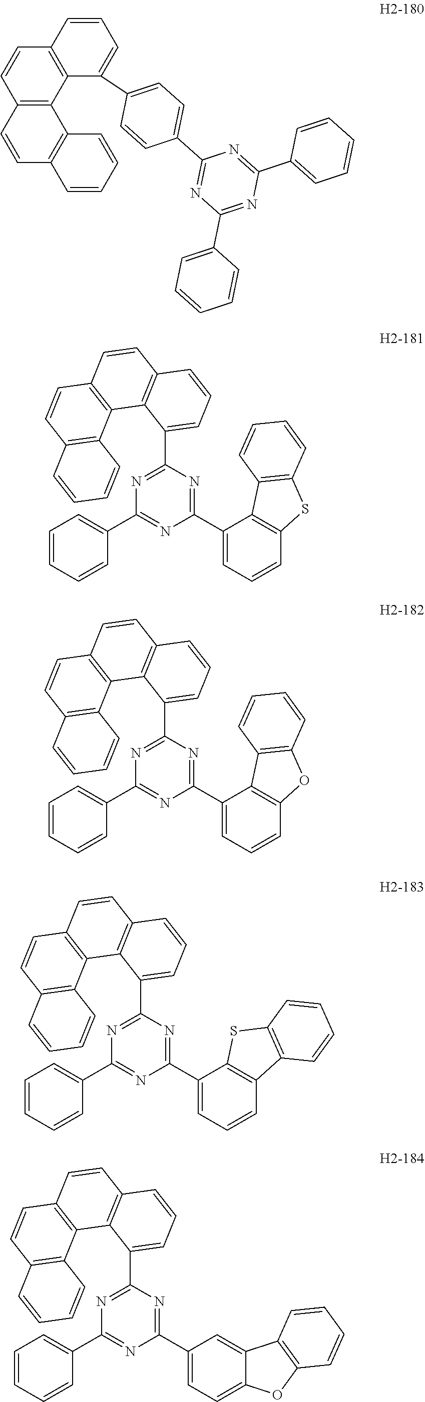

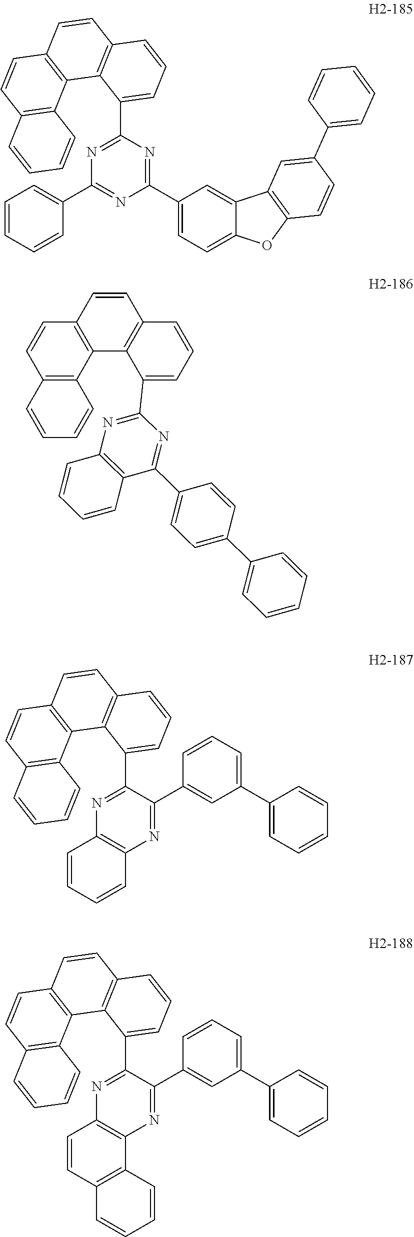 [SMILES: c1ccc(-c2ccc3oc4ccc(-c5nc(-c6ccccc6)nc(-c6cccc7ccc8ccc9ccccc9c8c67)n5)cc4c3c2)cc1 ; c1ccc(-c2ccc(-c3nc(-c4cccc5ccc6ccc7ccccc7c6c45)nc4ccccc34)cc2)cc1 ; c1ccc(-c2cccc(-c3nc4ccccc4nc3-c3cccc4ccc5ccc6ccccc6c5c34)c2)cc1 ; c1ccc(-c2cccc(-c3nc4c(ccc5ccccc54)nc3-c3cccc4ccc5ccc6ccccc6c5c34)c2)cc1]

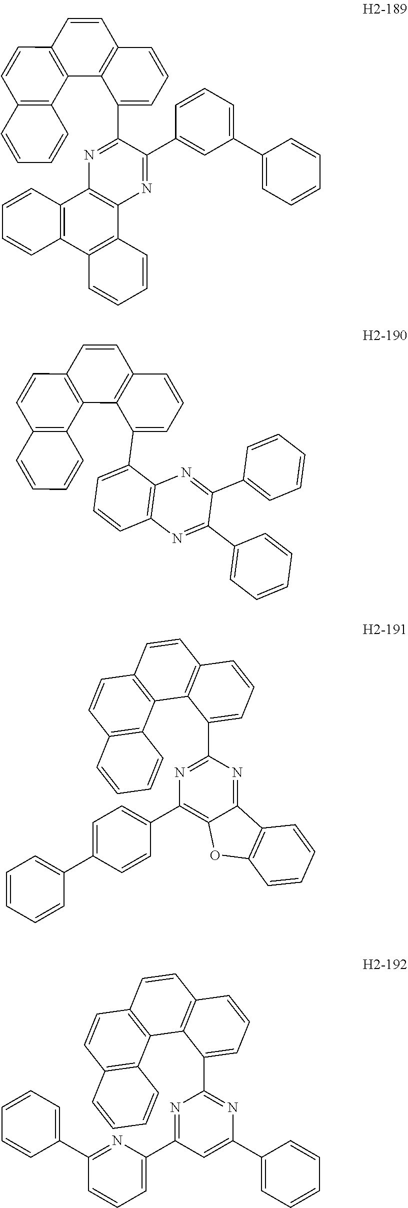

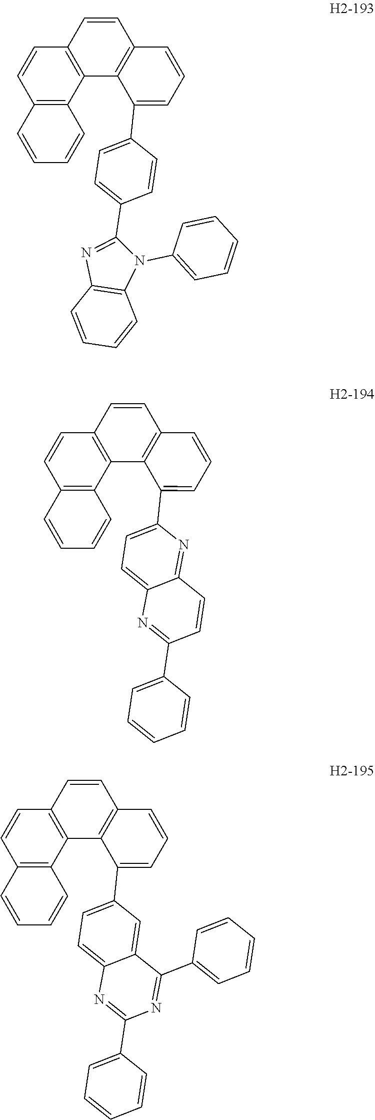 [SMILES: c1ccc(-n2c(-c3ccc(-c4cccc5ccc6ccc7ccccc7c6c45)cc3)nc3ccccc32)cc1 ; c1ccc(-c2ccc3nc(-c4cccc5ccc6ccc7ccccc7c6c45)ccc3n2)cc1 ; c1ccc(-c2nc(-c3ccccc3)c3cc(-c4cccc5ccc6ccc7ccccc7c6c45)ccc3n2)cc1]

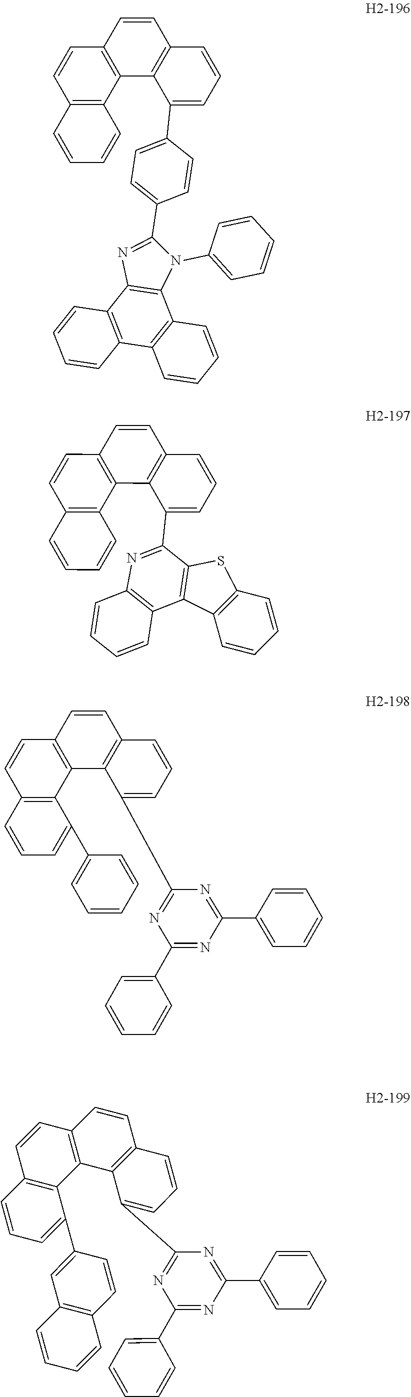 [SMILES: c1ccc(-n2c(-c3ccc(-c4cccc5ccc6ccc7ccccc7c6c45)cc3)nc3c4ccccc4c4ccccc4c32)cc1 ; c1ccc2c(c1)ccc1ccc3cccc(-c4nc5ccccc5c5c4sc4ccccc45)c3c12 ; c1ccc(-c2nc(-c3ccccc3)nc(-c3cccc4ccc5ccc6cccc(-c7ccccc7)c6c5c34)n2)cc1 ; c1ccc(-c2nc(-c3ccccc3)nc(-c3cccc4ccc5ccc6cccc(-c7ccc8ccccc8c7)c6c5c34)n2)cc1]

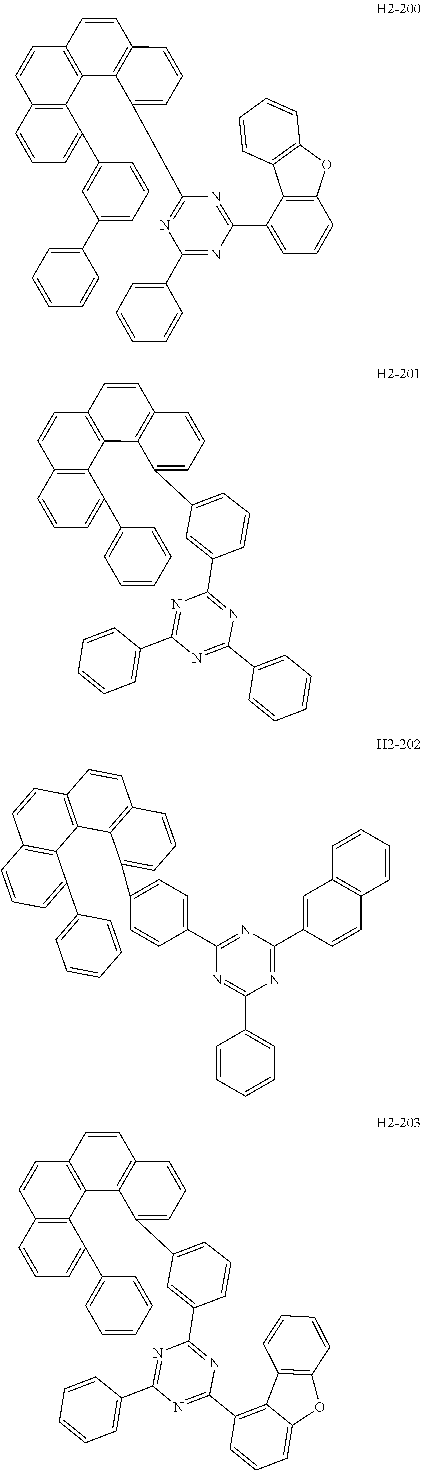

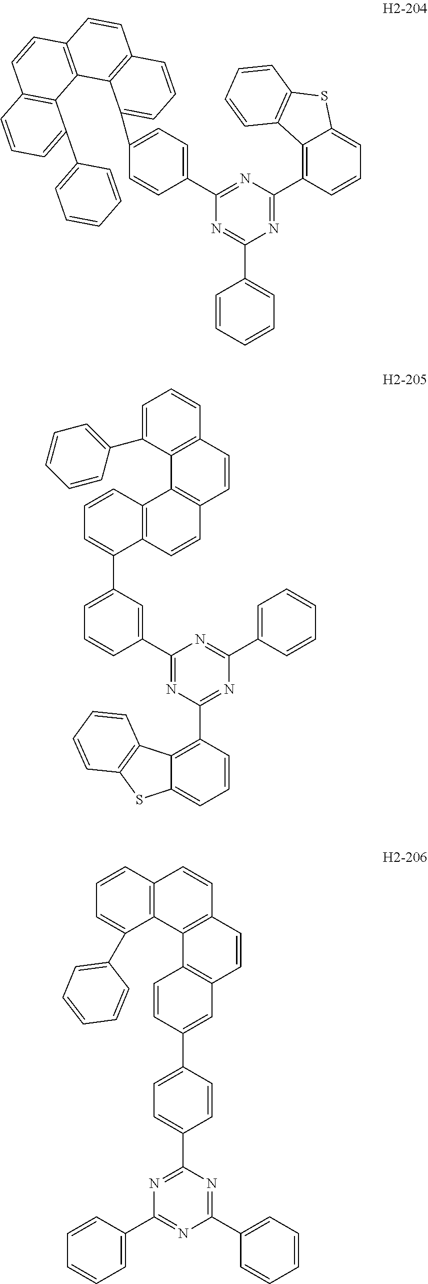 [SMILES: c1ccc(-c2nc(-c3ccc(-c4cccc5ccc6ccc7cccc(-c8ccccc8)c7c6c45)cc3)nc(-c3cccc4sc5ccccc5c34)n2)cc1 ; c1ccc(-c2nc(-c3cccc(-c4cccc5c4ccc4ccc6cccc(-c7ccccc7)c6c45)c3)nc(-c3cccc4sc5ccccc5c34)n2)cc1 ; c1ccc(-c2nc(-c3ccccc3)nc(-c3ccc(-c4ccc5c(ccc6ccc7cccc(-c8ccccc8)c7c65)c4)cc3)n2)cc1]

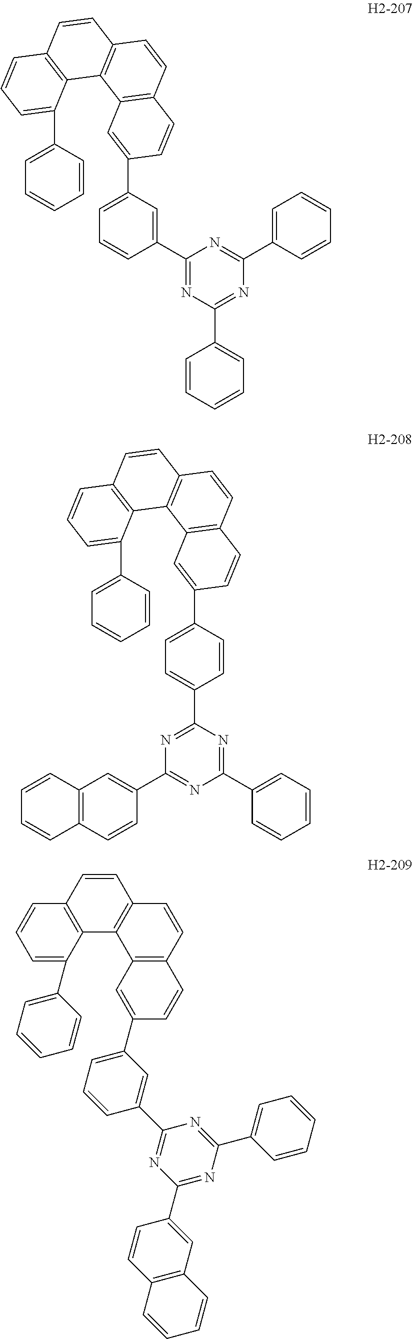

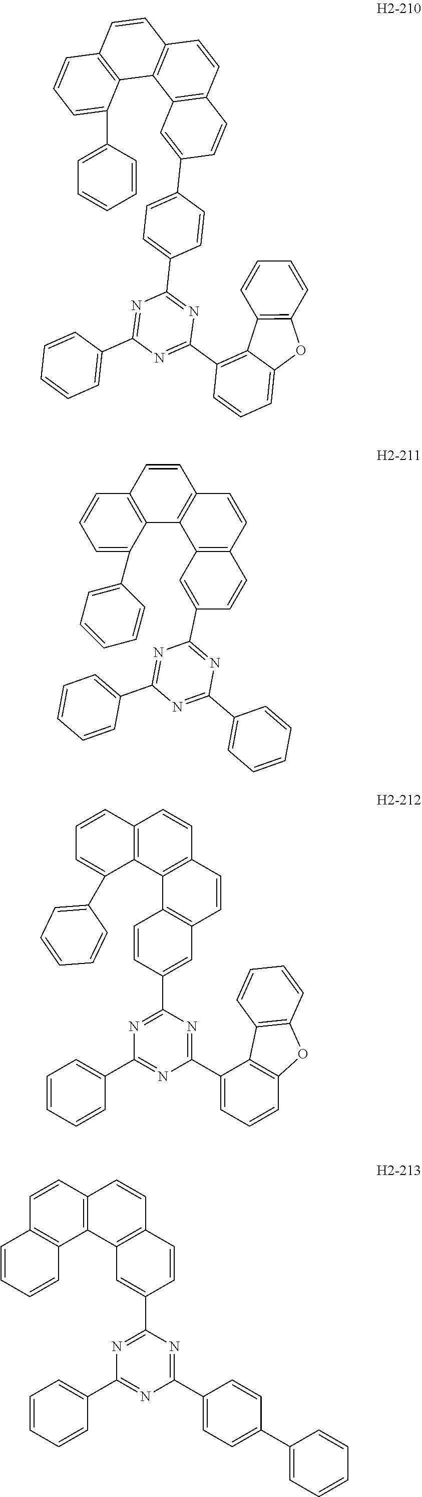 [SMILES: c1ccc(-c2nc(-c3ccc(-c4ccc5ccc6ccc7cccc(-c8ccccc8)c7c6c5c4)cc3)nc(-c3cccc4oc5ccccc5c34)n2)cc1 ; c1ccc(-c2nc(-c3ccccc3)nc(-c3ccc4ccc5ccc6cccc(-c7ccccc7)c6c5c4c3)n2)cc1 ; c1ccc(-c2nc(-c3ccc4c(ccc5ccc6cccc(-c7ccccc7)c6c54)c3)nc(-c3cccc4oc5ccccc5c34)n2)cc1 ; c1ccc(-c2ccc(-c3nc(-c4ccccc4)nc(-c4ccc5ccc6ccc7ccccc7c6c5c4)n3)cc2)cc1]

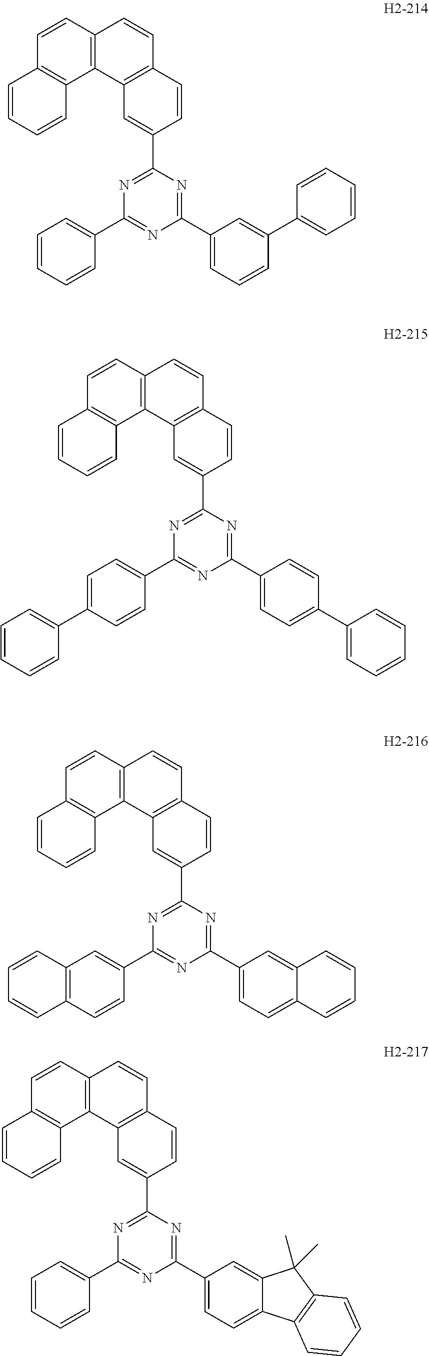 [SMILES: c1ccc(-c2cccc(-c3nc(-c4ccccc4)nc(-c4ccc5ccc6ccc7ccccc7c6c5c4)n3)c2)cc1 ; c1ccc(-c2ccc(-c3nc(-c4ccc(-c5ccccc5)cc4)nc(-c4ccc5ccc6ccc7ccccc7c6c5c4)n3)cc2)cc1 ; c1ccc2cc(-c3nc(-c4ccc5ccccc5c4)nc(-c4ccc5ccc6ccc7ccccc7c6c5c4)n3)ccc2c1 ; CC1(C)c2ccccc2-c2ccc(-c3nc(-c4ccccc4)nc(-c4ccc5ccc6ccc7ccccc7c6c5c4)n3)cc21]

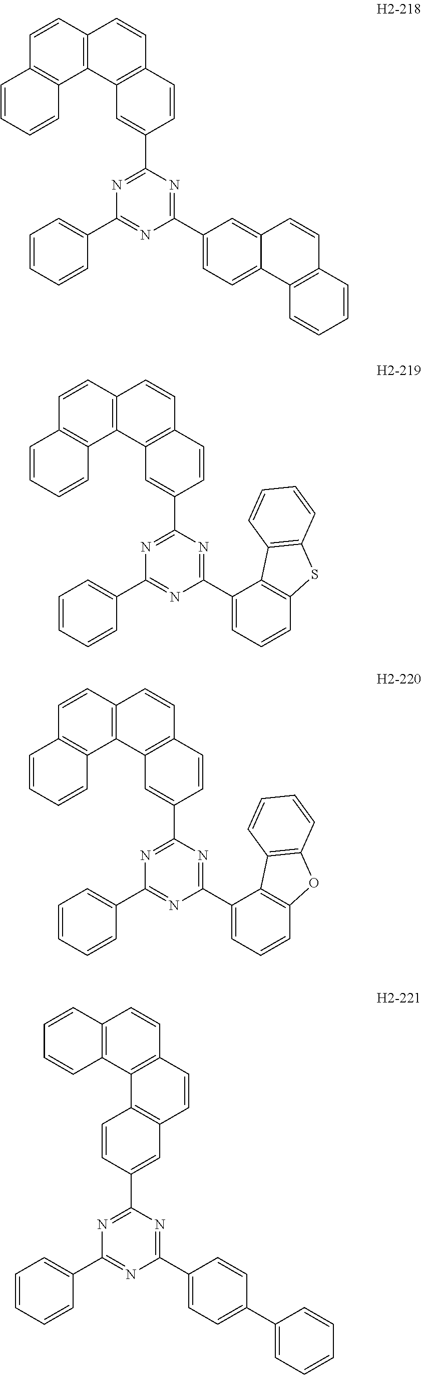

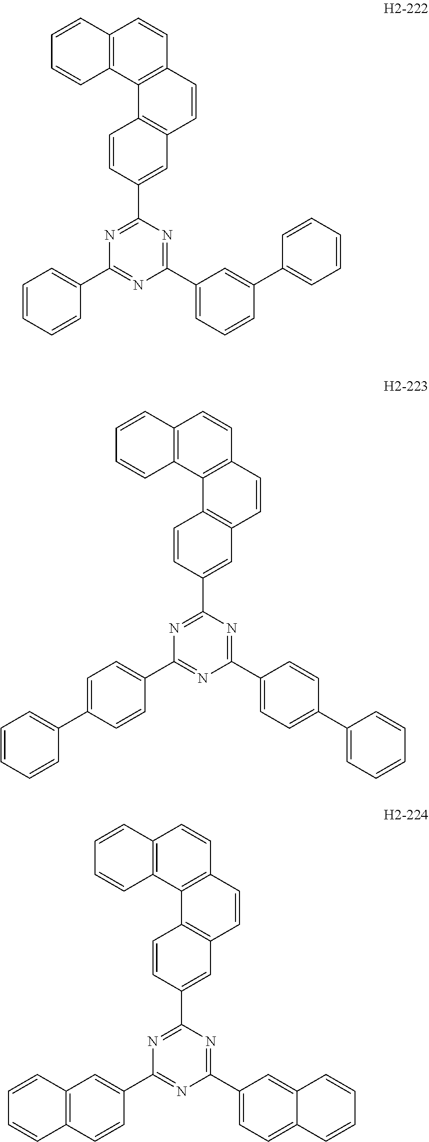 [SMILES: c1ccc(-c2cccc(-c3nc(-c4ccccc4)nc(-c4ccc5c(ccc6ccc7ccccc7c65)c4)n3)c2)cc1 ; c1ccc(-c2ccc(-c3nc(-c4ccc(-c5ccccc5)cc4)nc(-c4ccc5c(ccc6ccc7ccccc7c65)c4)n3)cc2)cc1 ; c1ccc2cc(-c3nc(-c4ccc5ccccc5c4)nc(-c4ccc5c(ccc6ccc7ccccc7c65)c4)n3)ccc2c1]

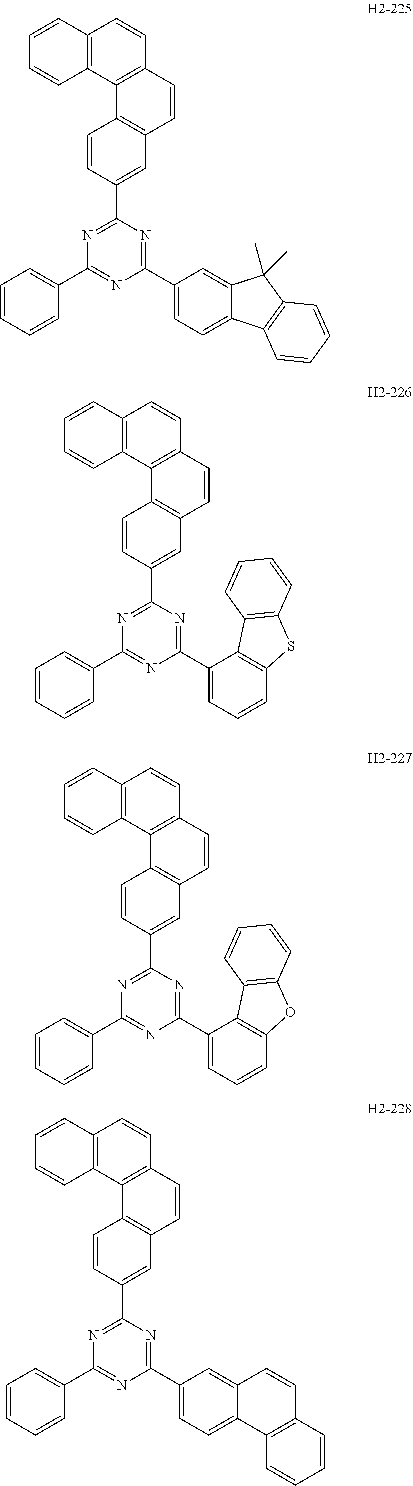

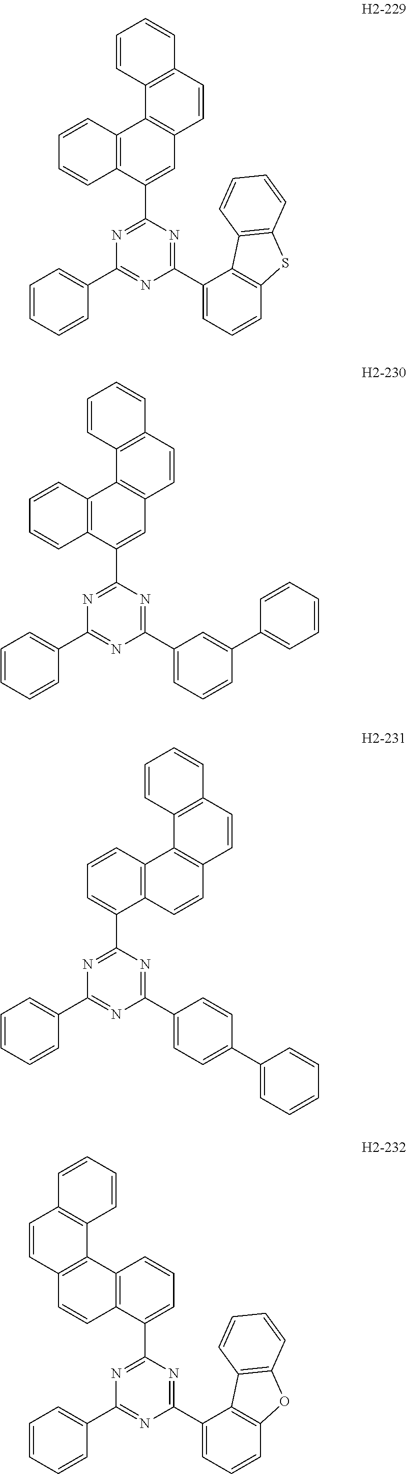 [SMILES: c1ccc(-c2nc(-c3cc4ccc5ccccc5c4c4ccccc34)nc(-c3cccc4sc5ccccc5c34)n2)cc1 ; c1ccc(-c2cccc(-c3nc(-c4ccccc4)nc(-c4cc5ccc6ccccc6c5c5ccccc45)n3)c2)cc1 ; c1ccc(-c2ccc(-c3nc(-c4ccccc4)nc(-c4cccc5c4ccc4ccc6ccccc6c45)n3)cc2)cc1 ; c1ccc(-c2nc(-c3cccc4c3ccc3ccc5ccccc5c34)nc(-c3cccc4oc5ccccc5c34)n2)cc1]

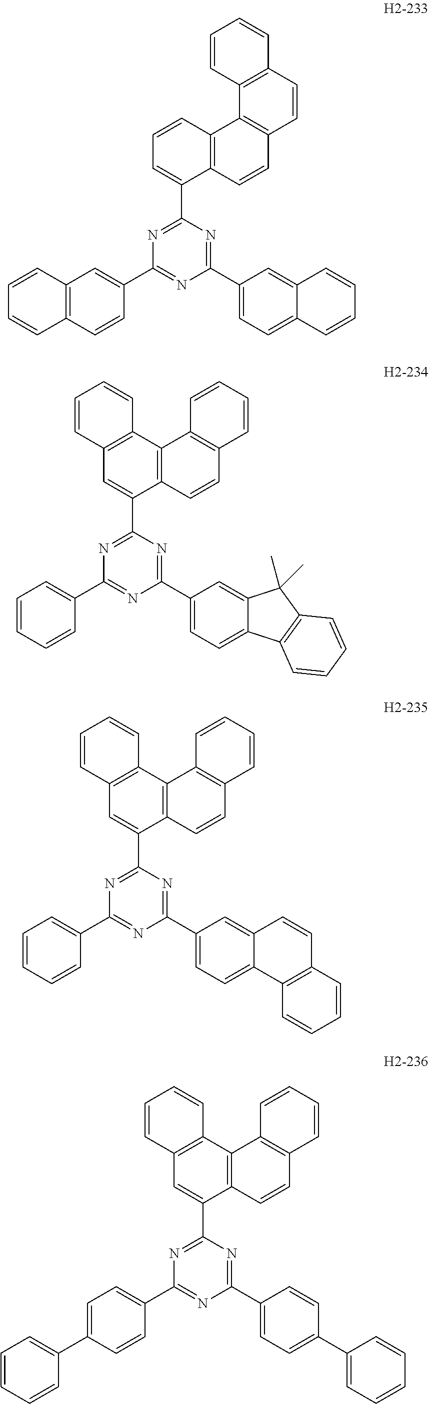 [SMILES: c1ccc2cc(-c3nc(-c4ccc5ccccc5c4)nc(-c4cccc5c4ccc4ccc6ccccc6c45)n3)ccc2c1 ; CC1(C)c2ccccc2-c2ccc(-c3nc(-c4ccccc4)nc(-c4cc5ccccc5c5c4ccc4ccccc45)n3)cc21 ; c1ccc(-c2nc(-c3ccc4c(ccc5ccccc54)c3)nc(-c3cc4ccccc4c4c3ccc3ccccc34)n2)cc1 ; c1ccc(-c2ccc(-c3nc(-c4ccc(-c5ccccc5)cc4)nc(-c4cc5ccccc5c5c4ccc4ccccc45)n3)cc2)cc1]

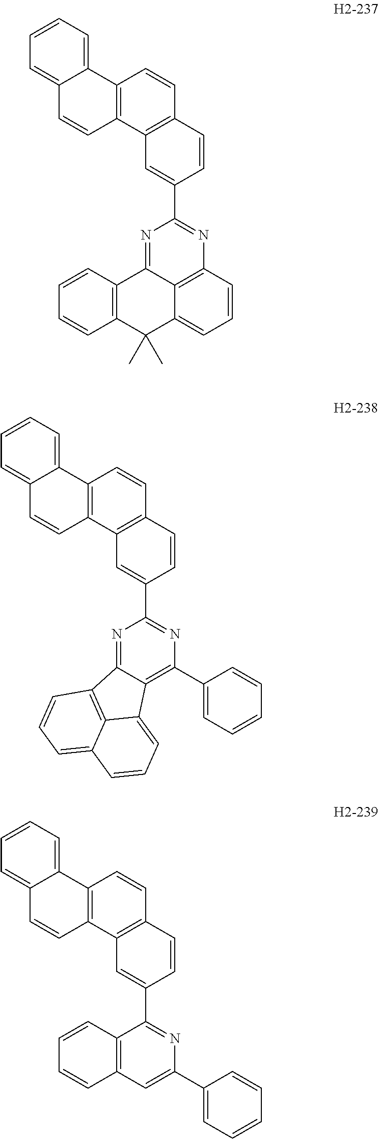 [SMILES: CC1(C)c2ccccc2-c2nc(-c3ccc4ccc5c6ccccc6ccc5c4c3)nc3cccc1c23 ; c1ccc(-c2nc(-c3ccc4ccc5c6ccccc6ccc5c4c3)nc3c2-c2cccc4cccc-3c24)cc1 ; c1ccc(-c2cc3ccccc3c(-c3ccc4ccc5c6ccccc6ccc5c4c3)n2)cc1]

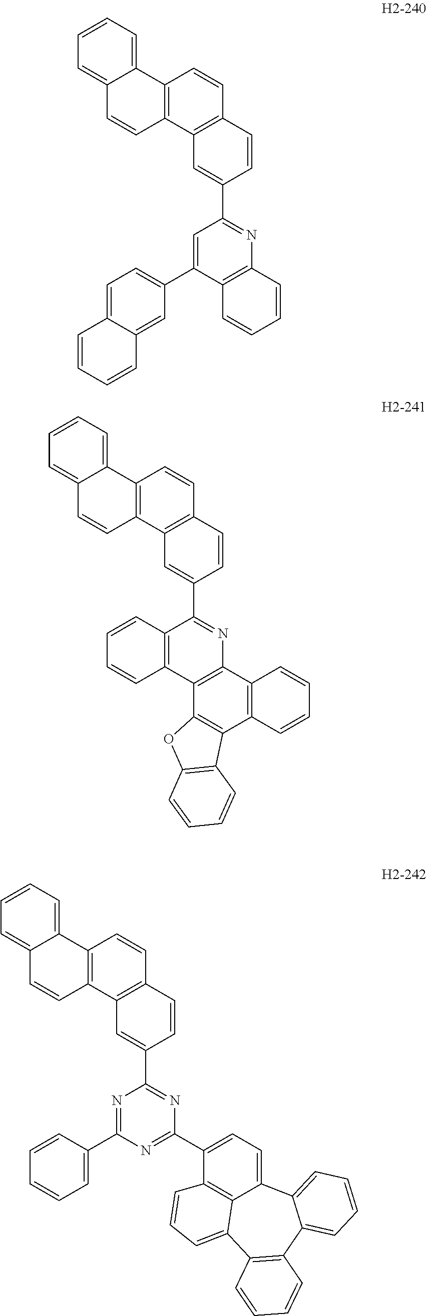

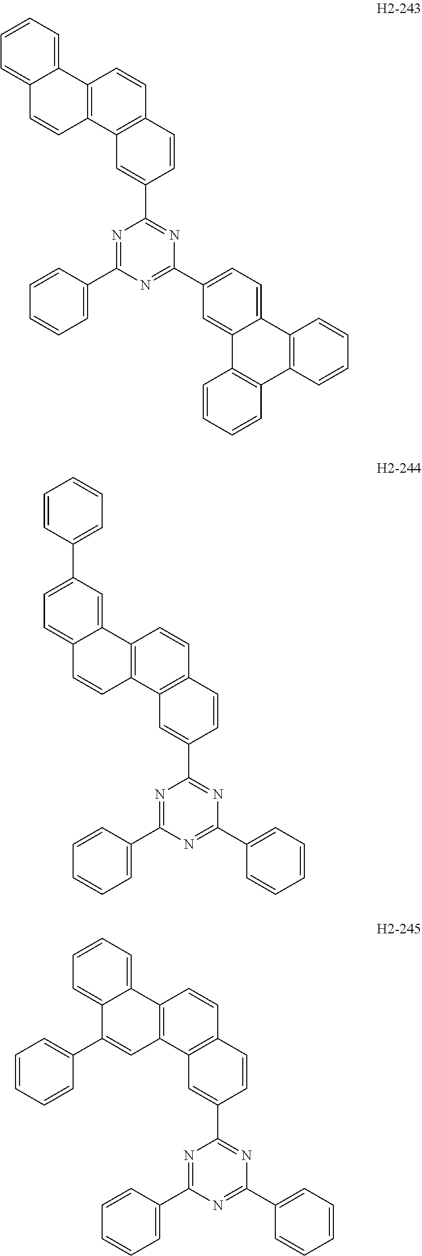

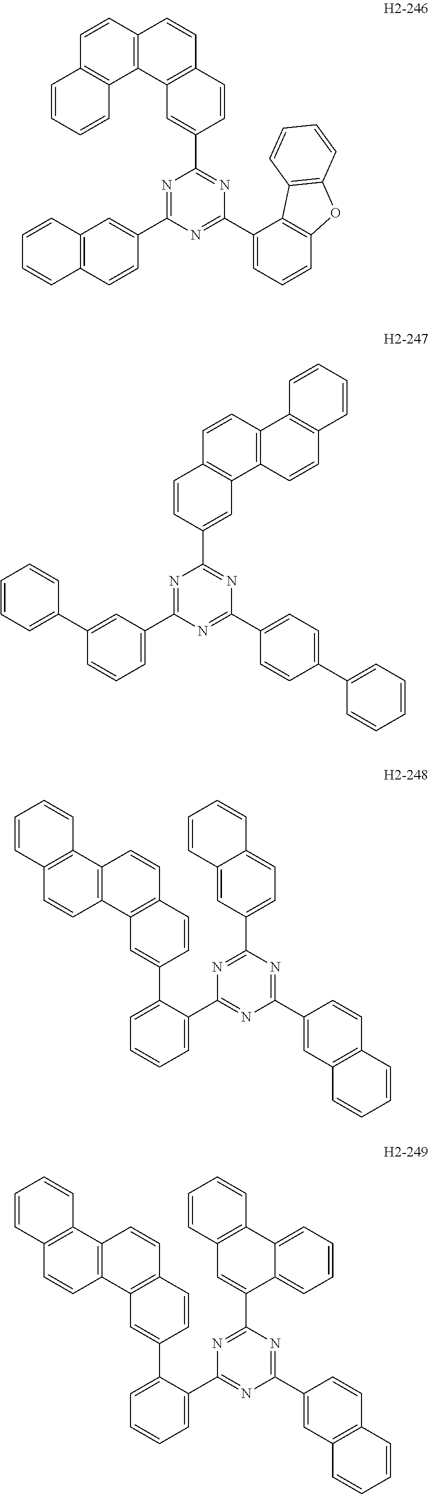 [SMILES: c1ccc2cc(-c3nc(-c4ccc5ccc6ccc7ccccc7c6c5c4)nc(-c4cccc5oc6ccccc6c45)n3)ccc2c1 ; c1ccc(-c2ccc(-c3nc(-c4cccc(-c5ccccc5)c4)nc(-c4ccc5ccc6c7ccccc7ccc6c5c4)n3)cc2)cc1 ; c1ccc(-c2nc(-c3ccc4ccccc4c3)nc(-c3ccc4ccccc4c3)n2)c(-c2ccc3ccc4c5ccccc5ccc4c3c2)c1 ; c1ccc(-c2nc(-c3ccc4ccccc4c3)nc(-c3cc4ccccc4c4ccccc34)n2)c(-c2ccc3ccc4c5ccccc5ccc4c3c2)c1]

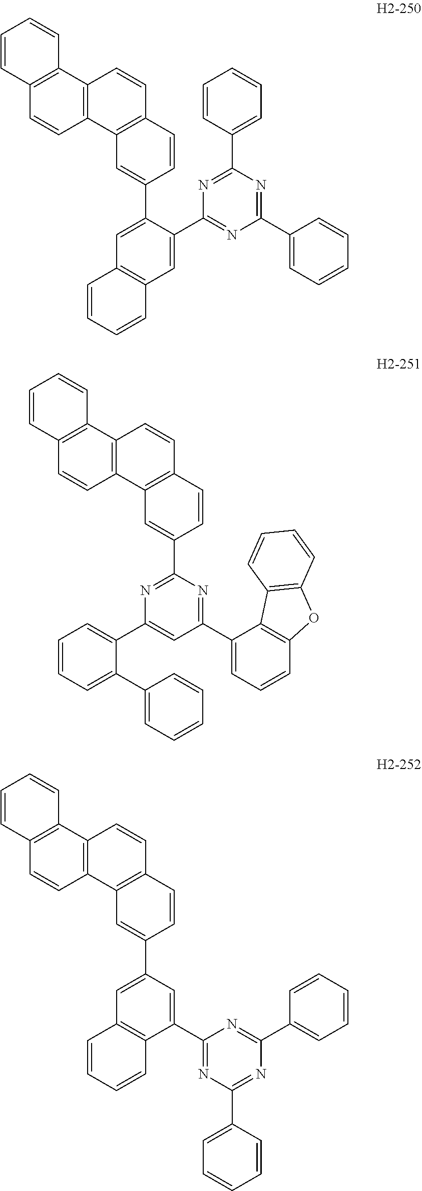 [SMILES: c1ccc(-c2nc(-c3ccccc3)nc(-c3cc4ccccc4cc3-c3ccc4ccc5c6ccccc6ccc5c4c3)n2)cc1 ; c1ccc(-c2ccccc2-c2cc(-c3cccc4oc5ccccc5c34)nc(-c3ccc4ccc5c6ccccc6ccc5c4c3)n2)cc1 ; c1ccc(-c2nc(-c3ccccc3)nc(-c3cc(-c4ccc5ccc6c7ccccc7ccc6c5c4)cc4ccccc34)n2)cc1]

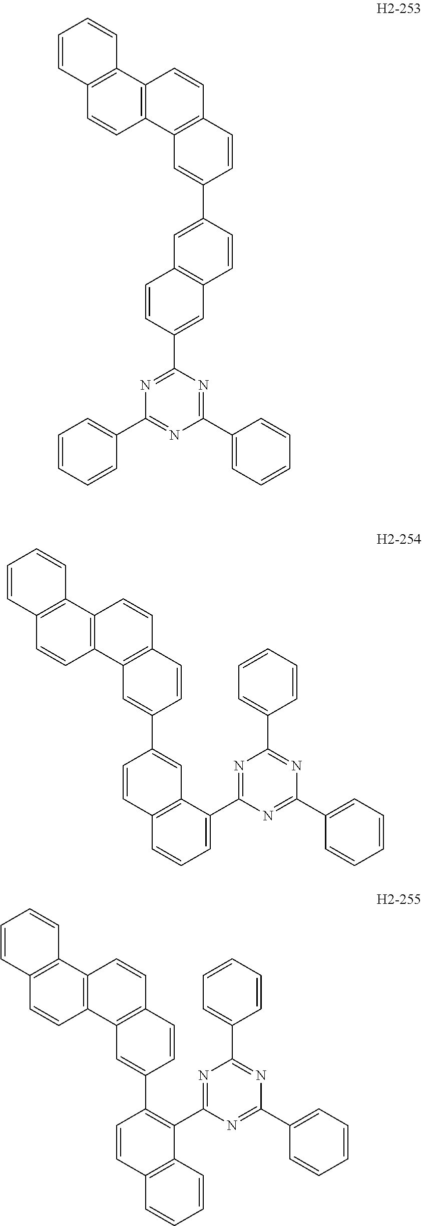 [SMILES: c1ccc(-c2nc(-c3ccccc3)nc(-c3ccc4cc(-c5ccc6ccc7c8ccccc8ccc7c6c5)ccc4c3)n2)cc1 ; c1ccc(-c2nc(-c3ccccc3)nc(-c3cccc4ccc(-c5ccc6ccc7c8ccccc8ccc7c6c5)cc34)n2)cc1 ; c1ccc(-c2nc(-c3ccccc3)nc(-c3c(-c4ccc5ccc6c7ccccc7ccc6c5c4)ccc4ccccc34)n2)cc1]

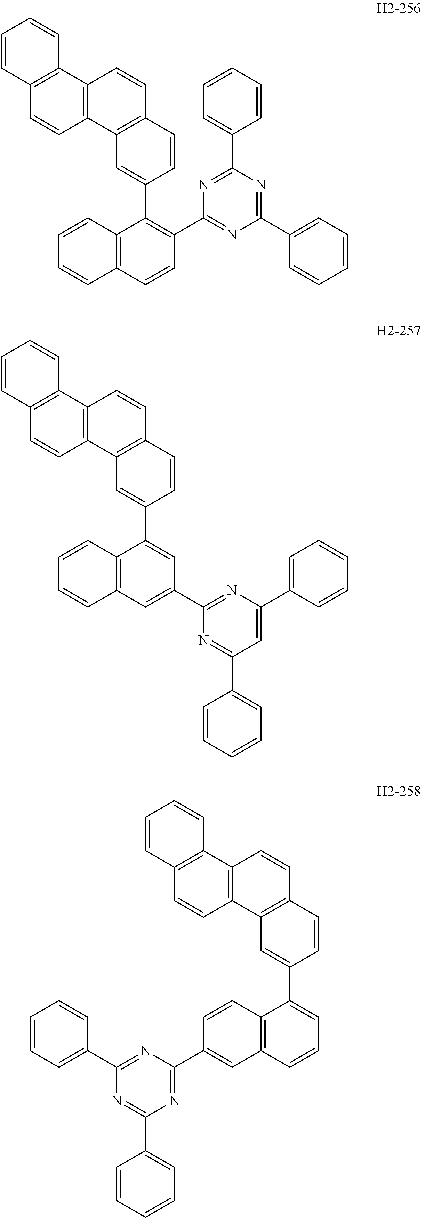 [SMILES: c1ccc(-c2nc(-c3ccccc3)nc(-c3ccc4ccccc4c3-c3ccc4ccc5c6ccccc6ccc5c4c3)n2)cc1 ; c1ccc(-c2cc(-c3ccccc3)nc(-c3cc(-c4ccc5ccc6c7ccccc7ccc6c5c4)c4ccccc4c3)n2)cc1 ; c1ccc(-c2nc(-c3ccccc3)nc(-c3ccc4c(-c5ccc6ccc7c8ccccc8ccc7c6c5)cccc4c3)n2)cc1]

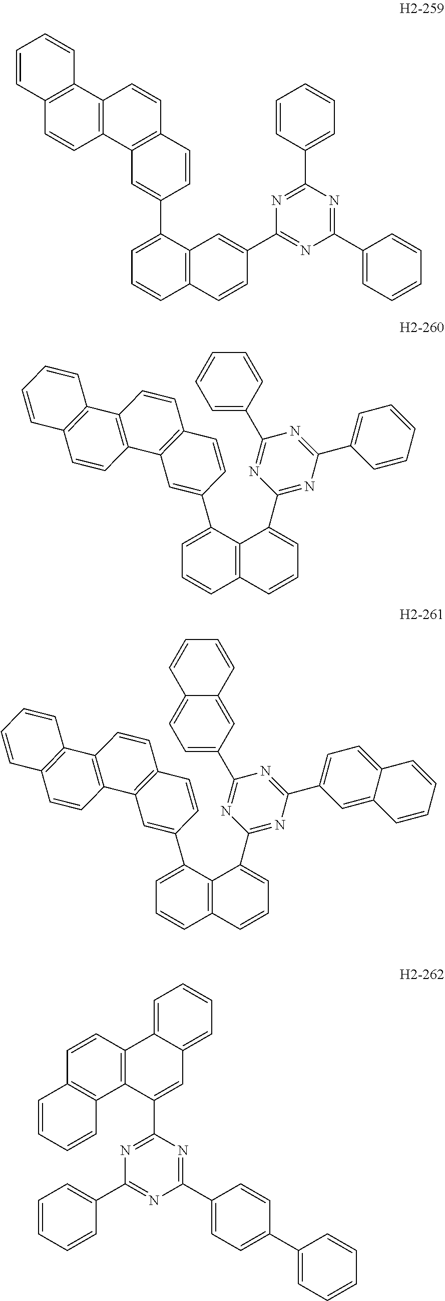

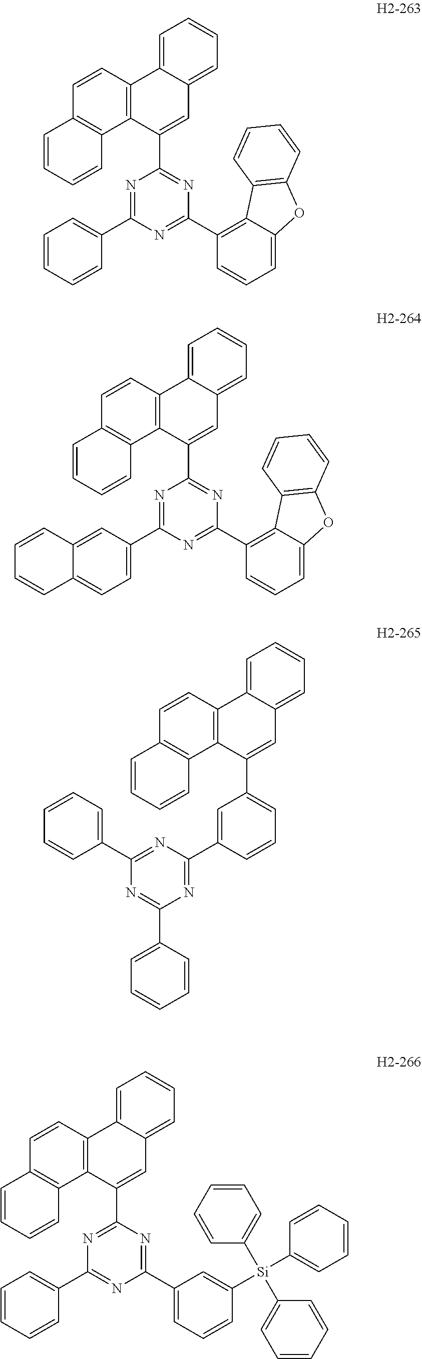 [SMILES: c1ccc(-c2nc(-c3cccc4oc5ccccc5c34)nc(-c3cc4ccccc4c4ccc5ccccc5c34)n2)cc1 ; c1ccc2cc(-c3nc(-c4cccc5oc6ccccc6c45)nc(-c4cc5ccccc5c5ccc6ccccc6c45)n3)ccc2c1 ; c1ccc(-c2nc(-c3ccccc3)nc(-c3cccc(-c4cc5ccccc5c5ccc6ccccc6c45)c3)n2)cc1 ; c1ccc(-c2nc(-c3cccc([Si](c4ccccc4)(c4ccccc4)c4ccccc4)c3)nc(-c3cc4ccccc4c4ccc5ccccc5c34)n2)cc1]

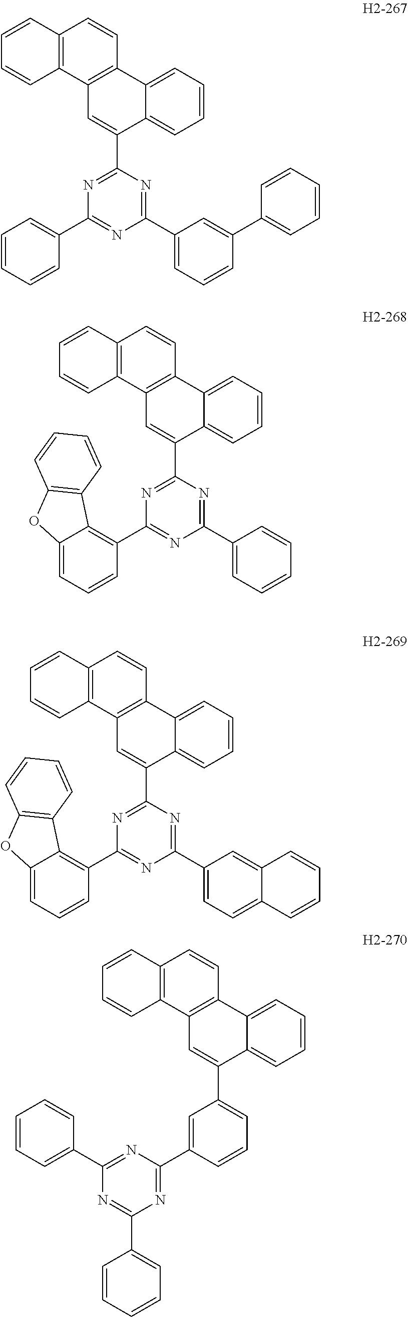 [SMILES: c1ccc(-c2cccc(-c3nc(-c4ccccc4)nc(-c4cc5c6ccccc6ccc5c5ccccc45)n3)c2)cc1 ; c1ccc(-c2nc(-c3cc4c5ccccc5ccc4c4ccccc34)nc(-c3cccc4oc5ccccc5c34)n2)cc1 ; c1ccc2cc(-c3nc(-c4cc5c6ccccc6ccc5c5ccccc45)nc(-c4cccc5oc6ccccc6c45)n3)ccc2c1 ; c1ccc(-c2nc(-c3ccccc3)nc(-c3cccc(-c4cc5c6ccccc6ccc5c5ccccc45)c3)n2)cc1]

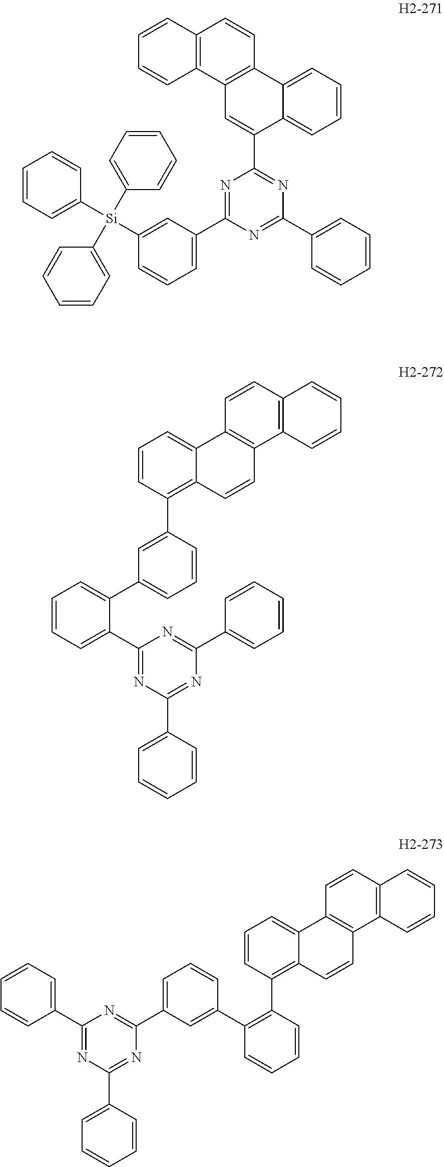 [SMILES: c1ccc(-c2nc(-c3cccc([Si](c4ccccc4)(c4ccccc4)c4ccccc4)c3)nc(-c3cc4c5ccccc5ccc4c4ccccc34)n2)cc1 ; c1ccc(-c2nc(-c3ccccc3)nc(-c3ccccc3-c3cccc(-c4cccc5c4ccc4c6ccccc6ccc54)c3)n2)cc1 ; c1ccc(-c2nc(-c3ccccc3)nc(-c3cccc(-c4ccccc4-c4cccc5c4ccc4c6ccccc6ccc54)c3)n2)cc1]

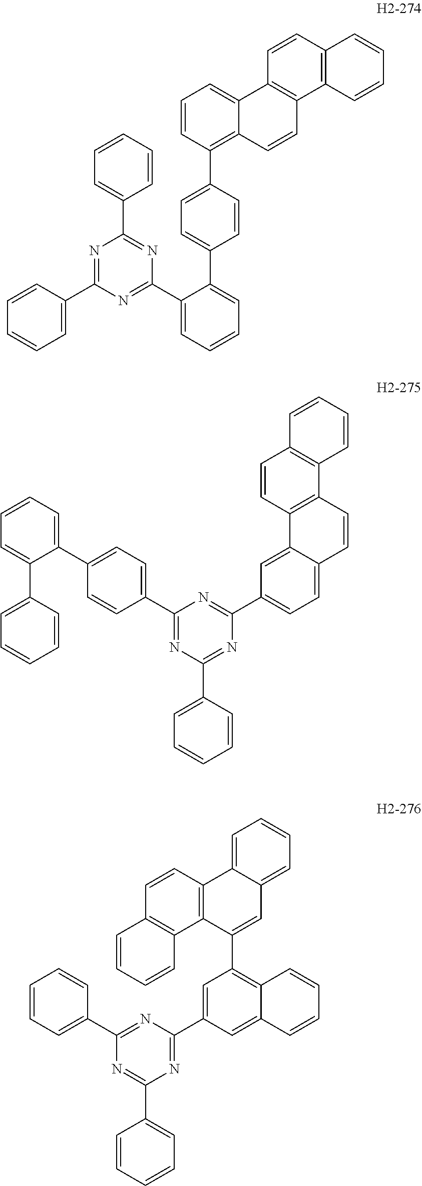 [SMILES: c1ccc(-c2nc(-c3ccccc3)nc(-c3ccccc3-c3ccc(-c4cccc5c4ccc4c6ccccc6ccc54)cc3)n2)cc1 ; c1ccc(-c2nc(-c3ccc(-c4ccccc4-c4ccccc4)cc3)nc(-c3ccc4ccc5c6ccccc6ccc5c4c3)n2)cc1 ; c1ccc(-c2nc(-c3ccccc3)nc(-c3cc(-c4cc5ccccc5c5ccc6ccccc6c45)c4ccccc4c3)n2)cc1]

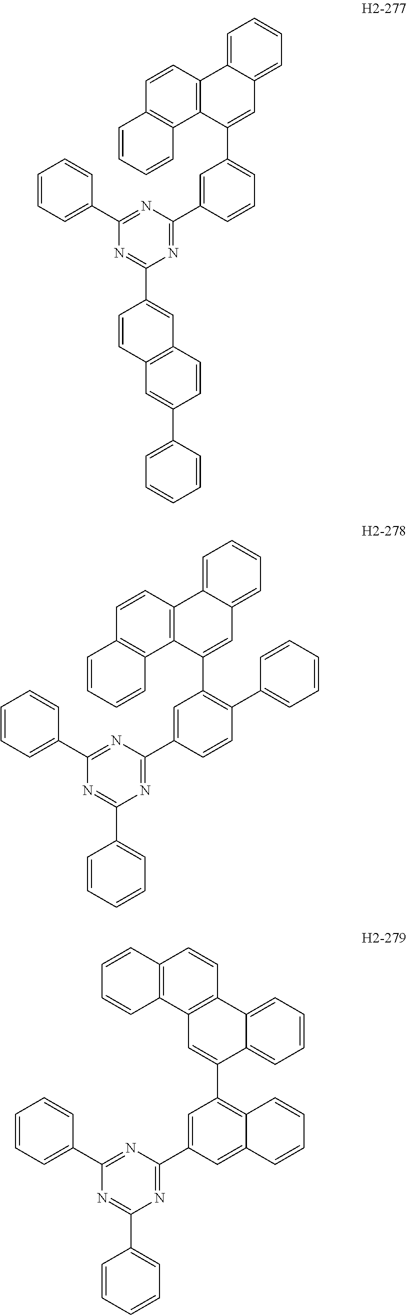

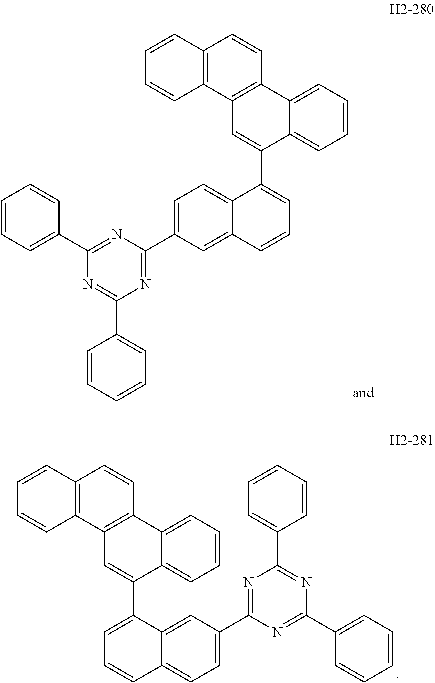 [SMILES: c1ccc(-c2nc(-c3ccccc3)nc(-c3ccc4c(-c5cc6c7ccccc7ccc6c6ccccc56)cccc4c3)n2)cc1 ; c1ccc(-c2nc(-c3ccccc3)nc(-c3ccc4cccc(-c5cc6c7ccccc7ccc6c6ccccc56)c4c3)n2)cc1]

16. An organic electroluminescent device comprising the organic electroluminescent compound according to claim 10.
